(19) United States
(12) Patent Application Publication
(10) Pub. No.: US 2022/0231229 A1
(43) Pub. Date: Jul. 21, 2022

(54) ORGANIC ELECTROLUMINESCENT COMPOUND, A PLURALITY OF HOST MATERIALS, AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Jeong-Eun Yang, Gyeonggi-do (KR); Sang-Hee CHO, Gyeonggi-do (KR); Hyo-Nim SHIN, Gyeonggi-do (KR); Hyun KIM, Gyeonggi-do (KR); Jeong-Hwan JEON, Gyeonggi-do (KR); Jin-Ri HONG, Gyeonggi-do (KR); Kyoung-Jin PARK, Gyeonggi-do (KR)

(21) Appl. No.: 17/550,562

(22) Filed: Dec. 14, 2021

(30) Foreign Application Priority Data
Dec. 28, 2020 (KR) 10-2020-0184746
Nov. 25, 2021 (KR) 10-2021-0164489

(51) Int. Cl. H01L 51/00; C07D 405/04; C07D 251/24; C07D 405/08; C07D 413/10; C07D 487/04

(52) U.S. Cl. CPC H01L 51/0067; H01L 51/0073; C07D 405/04; C07D 251/24; C07D 405/08; H01L 51/5016; H01L 51/0071; H01L 51/0072; C07D 487/04; H01L 2251/5384; C07D 413/10

(57) ABSTRACT

The present disclosure relates to an organic electroluminescent compound, a plurality of host materials, and an organic electroluminescent device comprising the same. By comprising the organic electroluminescent compound according to the present disclosure as a single host material, or a specific combination of compounds according to the present disclosure as a plurality of host materials, it is possible to produce an organic electroluminescent device having improved driving voltage, luminous efficiency, and/or lifetime properties.

### TECHNICAL FIELD

The present disclosure relates to an organic electroluminescent compound, a plurality of host materials, and an organic electroluminescent device comprising the same.

### BACKGROUND ART

A small molecular green organic electroluminescent device (OLED) was first developed by Tang, et al., of Eastman Kodak in 1987 by using TPD/ALq3 bi-layer consisting of a light-emitting layer and a charge transport layer. Thereafter, the development of OLEDs was rapidly effected and OLEDs have been commercialized. At present, OLEDs primarily use phosphorescent materials having excellent luminous efficiency in panel implementation. However, in many applications such as TVs and lightings, the lifetime of OLEDs is insufficient and higher efficiency of OLEDs is still required. Typically, the higher the luminance of an OLED, the shorter the lifetime that the OLED has. Therefore, an OLED having high luminous efficiency and/or long lifetime characteristics is required for long time use and high resolution of a display.

In order to enhance luminous efficiency, driving voltage and/or lifetime, various materials or concepts for an organic layer of an OLED have been proposed. However, they were not satisfied in practical use.

Meanwhile, Korean Patent Application Laying-Open No. 2010-0133467 discloses a fluorene derivative compound. However, the aforementioned reference does not specifically disclose a specific compound, or a specific combination of host materials claimed in the present disclosure. In addition, it is required to develop a light-emitting material having improved performances, for example, low driving voltage, high efficiency, and/or improved lifetime properties, as compared with the compounds disclosed in the aforementioned reference.

### DISCLOSURE OF INVENTION

### Technical Problem

The objective of the present disclosure is to provide an organic electroluminescent compound having a new structure suitable for applying it to an organic electroluminescent device. Another objective of the present disclosure is to provide an improved organic electroluminescent material capable of providing an organic electroluminescent device having improved driving voltage, luminous efficiency and/or lifetime properties. Still another objective of the present disclosure is to provide an organic electroluminescent device having improved driving voltage, luminous efficiency and/or lifetime properties by comprising a specific combination of compounds as host materials.

### Solution to Problem

As a result of intensive studies to solve the technical problems, the present inventors found that the above objective can be achieved by an organic electroluminescent compound represented by the following formula 4. In addition, the present inventors found that the above objective can be achieved by a plurality of host materials comprising a first host material comprising a compound represented by the following formula 1, and a second host material comprising a compound represented by the following formula 2 or 3.

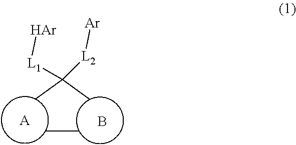

In formula 1,

ring A and ring B, each independently, represent a substituted or unsubstituted (C6-C30)arene, or a substituted or unsubstituted (3- to 30-membered)heteroarene;

L₁ and L₂, each independently, represent a single bond, a substituted or unsubstituted (C6-C30)arylene, or a substituted or unsubstituted (3- to 30-membered)heteroarylene;

Ar represents a substituted or unsubstituted (C6-C30)aryl, or a substituted or unsubstituted (3- to 30-membered)heteroaryl; and

HAr represents a substituted or unsubstituted (3- to 20-membered)heteroaryl containing a nitrogen atom(s).

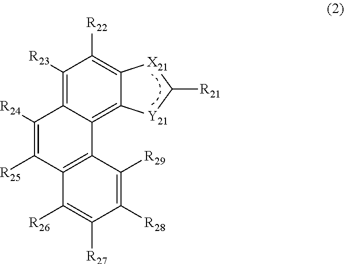

In formula 2,

X₂₁ and Y₂₁, each independently, represent —N═, —NR₃₁—, —O—, or —S—, with the proviso that any one of X₂₁ and Y₂₁ represents —N═ and the other one of X₂₁ and Y₂₁ represents —NR₃₁—, —O—, or —S—;

R₂₁ and R₃₁, each independently, represent a substituted or unsubstituted (C6-C30)aryl, or a substituted or unsubstituted (3- to 30-membered)heteroaryl;

R₂₂ to R₂₉, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), -L-NR₁R₂, or -L₂₁-Ar₂₁; or may be linked to an adjacent substituent to form a ring(s); with the proviso that at least one of R₂₂ to R₂₉ represents -L₂₁-Ar₂₁;

L₂₁, each independently, represents a single bond, a substituted or unsubstituted (C6-C30)arylene, or a substituted or unsubstituted (3- to 30-membered)heteroarylene;

Ar₂₁, each independently, represents a substituted or unsubstituted fused ring group of a (C3-C30) aliphatic ring(s) and a (C6-C30) aromatic ring(s), a substituted or unsubstituted (C6-C30)aryl, a substituted or unsubstituted (3- to 30-membered)heteroaryl, or —NR₃₂R₃₃;

R₃₂ and R₃₃, each independently, represent a substituted or unsubstituted (C1-C30)alkyl, a substituted or unsubstituted (C2-C30)alkenyl, a substituted or unsubstituted (C6-C30)aryl, a substituted or unsubstituted (3- to 30-membered)heteroaryl, or a substituted or unsubstituted fused ring group of a (C3-C30) aliphatic ring(s) and a (C6-C30) aromatic ring(s);

L represents a single bond, a substituted or unsubstituted (C6-C30)arylene, a substituted or unsubstituted (3- to 30-membered)heteroarylene, a substituted or unsubstituted divalent (C2-C30) aliphatic hydrocarbon group, or a substituted or unsubstituted divalent fused ring group of a (C3-C30) aliphatic ring(s) and a (C6-C30) aromatic ring(s); and

R₁ and R₂, each independently, represent hydrogen, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl.

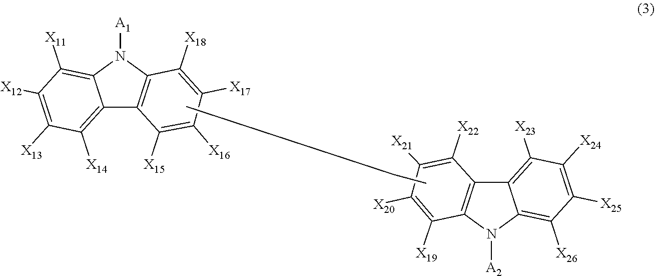

In formula 3,

A₁ and A₂, each independently, represent a substituted or unsubstituted (C6-C30)aryl, a substituted or unsubstituted dibenzofuranyl, a substituted or unsubstituted dibenzothiophenyl, or a substituted or unsubstituted carbazolyl;

one of X₁₅ to X₁₈ and one of X₁₉ to X₂₂ are linked to each other to form a single bond; and

the remaining X₁₅ to X₂₂ which do not form a single bond, X₁₁ to X₁₄, and X₂₃ to X₂₆, each independently, represent hydrogen, deuterium, a substituted or unsubstituted (C6-C30)aryl, or a substituted or unsubstituted (3- to 30-membered)heteroaryl; or may be linked to an adjacent substituent to form a ring(s).

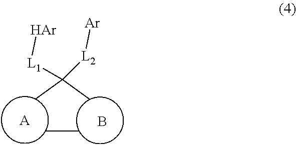

In formula 4,

ring A and ring B, each independently, represent a substituted or unsubstituted benzene ring, a substituted or unsubstituted naphthalene ring, or a substituted or unsubstituted phenanthrene ring;

L₁ represents a single bond;

L₂ represents a single bond, or a substituted or unsubstituted (C6-C30)arylene;

Ar represents a substituted or unsubstituted (C6-C30)aryl, a substituted or unsubstituted dibenzofuranyl, a substituted or unsubstituted dibenzothiophenyl, or a substituted or unsubstituted carbazolyl;

HAr represents a substituted or unsubstituted (3- to 20-membered)heteroaryl containing a nitrogen atom(s);

with the proviso that HAr is not a heteroaryl substituted with a substituent

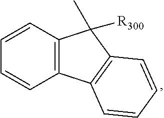

in which R₃₀₀ represents a (C1-C30)alkyl, or a (C6-C30)aryl, and * represents a bonding site of the substituent; and

the compound represented by formula 4 is not the following compounds:

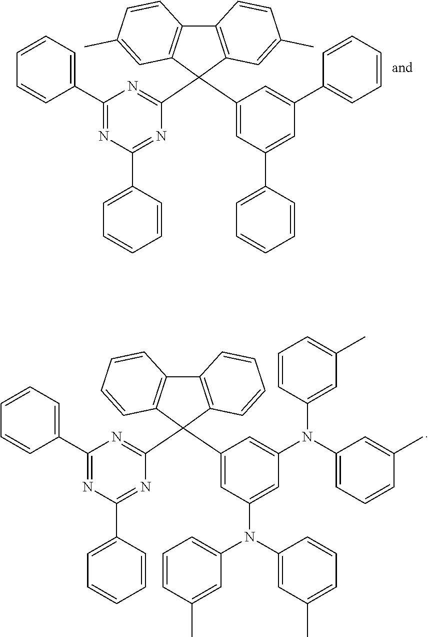 [SMILES: Cc1ccc2c(c1)C(c1cc(-c3ccccc3)cc(-c3ccccc3)c1)(c1nc(-c3ccccc3)nc(-c3ccccc3)n1)c1cc(C)ccc1-2 ; Cc1cccc(N(c2cccc(C)c2)c2cc(N(c3cccc(C)c3)c3cccc(C)c3)cc(C3(c4nc(-c5ccccc5)nc(-c5ccccc5)n4)c4ccccc4-c4ccccc43)c2)c1]

### Advantageous Effects of Invention

The organic electroluminescent compound according to the present disclosure exhibits performances suitable for using it in an organic electroluminescent device. In addition, an organic electroluminescent device having low driving voltage, high luminous efficiency and/or excellent lifetime properties compared to conventional organic electroluminescent devices is provided by comprising the compound according to the present disclosure as a single host material, or by comprising a specific combination of compounds according to the present disclosure as a plurality of host materials, and it is possible to produce a display system or a lighting system using the same.

### MODE FOR THE INVENTION

Hereinafter, the present disclosure will be described in detail. However, the following description is intended to explain the present disclosure and is not meant in any way to restrict the scope of the present disclosure.

The term “organic electroluminescent compound” in the present disclosure means a compound that may be used in an organic electroluminescent device, and may be comprised in any layer constituting an organic electroluminescent device, as necessary.

The term “an organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

The term “a plurality of host materials” in the present disclosure means a host material comprising a combination of at least two compounds, which may be comprised in any light-emitting layer constituting an organic electroluminescent device. It may mean both a material before being comprised in an organic electroluminescent device (for example, before vapor deposition) and a material after being comprised in an organic electroluminescent device (for example, after vapor deposition). For example, the plurality of host materials of the present disclosure is a combination of at least two host materials, and may selectively further comprise conventional materials comprised in an organic electroluminescent material. At least two compounds comprised in the plurality of host materials of the present disclosure may be comprised together in one light-emitting layer or may respectively be comprised in different light-emitting layers. For example, the at least two host materials may be mixture-evaporated or co-evaporated, or may be individually evaporated.

Herein, the term “(C1-C30)alkyl” is meant to be a linear or branched alkyl having 1 to 30 carbon atoms constituting the chain, in which the number of carbon atoms is preferably 1 to 10, and more preferably 1 to 6. The above alkyl may include methyl, ethyl, n-propyl, isopropyl, n-butyl, isobutyl, tert-butyl, sec-butyl, etc. The term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. The term “(3- to 7-membered)heterocycloalkyl” is meant to be a cycloalkyl having 3 to 7 ring backbone atoms, and including at least one heteroatom selected from the group consisting of B, N, O, S, Si, and P, and preferably the group consisting of O, S, and N. The above heterocycloalkyl may include tetrahydrofuran, pyrrolidine, thiolan, tetrahydropyran, etc. The term “(C6-C30)aryl,” “(C6-C30)arylene,” or “(C6-C30)arene” is meant to be a monocyclic or fused ring radical derived from an aromatic hydrocarbon having 6 to 30 ring backbone carbon atoms. The above aryl, arylene, or arene may be partially saturated, and may comprise a spiro structure. The above aryl may include phenyl, biphenyl, terphenyl, naphthyl, binaphthyl, phenylnaphthyl, naphthylphenyl, fluorenyl, phenylfluorenyl, diphenylfluorenyl, benzofluorenyl, dibenzofluorenyl, phenanthrenyl, phenylphenanthrenyl, benzophenanthrenyl, anthracenyl, indenyl, triphenylenyl, pyrenyl, tetracenyl, perylenyl, chrysenyl, naphthacenyl, fluoranthenyl, spirobifluorenyl, spiro[fluorene-benzofluoren]yl, spiro[cyclopentane-fluoren]yl, spiro[dihydroindene-fluoren]yl, azulenyl, tetramethyldihydrophenanthrenyl, etc. Specifically, the above aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenyl, 3-biphenyl, 4-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The term “(3- to 30-membered)heteroaryl(ene)” is meant to be an aryl or an arylene having 3 to 30 ring backbone atoms, and including at least one, preferably 1 to 4 heteroatoms selected from the group consisting of B, N, O, S, Si, and P. The above heteroaryl(ene) may be a monocyclic ring, or a fused ring condensed with at least one benzene ring; may be partially saturated; may be one formed by linking at least one heteroaryl or aryl group to a heteroaryl group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, and pyridazinyl, and a fused ring-type heteroaryl such as benzofuranyl, benzothiophenyl, isobenzofuranyl, dibenzofuranyl, dibenzothiophenyl, dibenzoselenophenyl, naphthobenzofuranyl, naphthobenzothiophenyl, benzofuroquinolinyl, benzofuroquinazolinyl, benzofuronaphthyridinyl, benzofuropyrimidinyl, naphthofuropyrimidinyl, benzothienoquinolinyl, benzothienoquinazolinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, benzocarbazolyl, dibenzocarbazolyl, phenoxazinyl, phenanthridinyl, benzodioxolyl, dihydroacridinyl, benzotriazolphenazinyl, imidazopyridyl, chromenoquinazolinyl, thiochromenoquinazolinyl, dimethylbenzoperimidinyl, indolocarbazolyl, indenocarbazolyl, etc. More specifically, the above heteroaryl may include 1-pyrrolyl, 2-pyrrolyl, 3-pyrrolyl, pyrazinyl, 2-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indolyl, 5-indolyl, 8-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthrdinyl, 3-phenanthridinyl, 4-phenanthrdinyl, 6-phenanthrdinyl, 7-phenanthridinyl, 8-phenanthridinyl, 9-phenanthridinyl, 10-phenanthridinyl, 1-acridinyl, 2-acridinyl, 3-acridinyl, 4-acrdinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrmidinyl, 7-benzofuro[3,2-d]pyrmidinyl, 8-benzofuro[3,2-d]pyrmidinyl, 9-benzofuro[3,2-d]pyrmidinyl, 2-benzothio[3,2-d]pyrimidinyl, 6-benzothio[3,2-d]pyrmidinyl, 7-benzothio[3,2-d]pyrmidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Furthermore, “halogen” includes F, Cl, Br, and I.

In addition, “ortho (o-),” “meta (m-),” and “para (p-)” are prefixes, which represent the relative positions of substituents respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

Herein, ‘substituted’ in the expression “substituted or unsubstituted” means that a hydrogen atom in a certain functional group is replaced with another atom or another functional group, i.e., a substituent, and also includes that the hydrogen atom is replaced with a group formed by a linkage of two or more substituents of the above substituents. For example, the “group formed by a linkage of two or more substituents” may be pyridine-triazine. That is, pyridine-triazine may be interpreted as a heteroaryl substituent, or as substituents in which two heteroaryl substituents are linked. Herein, the substituent(s) of the substituted alkyl, the substituted alkenyl, the substituted aryl, the substituted arene, the substituted arylene, the substituted heteroaryl, the substituted heteroarene, the substituted heteroarylene, the substituted dibenzofuranyl, the substituted dibenzothiophenyl, the substituted carbazolyl, the substituted cycloalkyl, the substituted alkoxy, the substituted trialkylsilyl, the substituted dialkylarylsilyl, the substituted alkyldiarylsilyl, the substituted triarylsilyl, the substituted fused ring group of an aliphatic ring(s) and an aromatic ring(s), the substituted divalent aliphatic hydrocarbon group, or the substituted divalent fused ring group of a aliphatic ring(s) and an aromatic ring(s), each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with a (C6-C30)aryl(s); a (C6-C30)aryl unsubstituted or substituted with a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a fused ring group of a (C3-C30) aliphatic ring(s) and a (C6-C30) aromatic ring(s); an amino; a mono- or di-(C1-C30)alkylamino; a mono- or di-(C2-C30)alkenylamino; a (C1-C30)alkyl(C2-C30)alkenylamino; a substituted or unsubstituted mono- or di-(C6-C30)arylamino; a (C1-C30)alkyl(C6-C30)arylamino; a mono- or di-(3- to 30-membered)heteroarylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphinyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl, in which the substituent(s) may be further substituted with deuterium. According to one embodiment of the present disclosure, the substituent(s), each independently, are at least one selected from the group consisting of deuterium; a (C1-C20)alkyl; a (5- to 25-membered)heteroaryl unsubstituted or substituted with a (C6-C25)aryl(s); a (C6-C25)aryl unsubstituted or substituted with a (5- to 25-membered)heteroaryl(s); and a tri(C6-C25)arylsilyl, in which the substituent(s) may be further substituted with deuterium. According to another embodiment of the present disclosure, the substituent(s), each independently, are at least one selected from the group consisting of deuterium; a (C1-C10)alkyl; a (5- to 25-membered)heteroaryl unsubstituted or substituted with a (C6-C18)aryl(s); a (C6-C20)aryl unsubstituted or substituted with a (5- to 20-membered)heteroaryl(s); and a tri(C6-C18)arylsilyl, in which the substituent(s) may be further substituted with deuterium. For example, the substituent(s) may be at least one selected from the group consisting of deuterium; a methyl; a phenyl unsubstituted or substituted with a dibenzofuranyl(s); a naphthyl unsubstituted or substituted with a dibenzofuranyl(s); a biphenyl unsubstituted or substituted with a dibenzofuranyl(s); a phenanthrenyl; a chrysenyl; a triphenylenyl; a pyridyl unsubstituted or substituted with a phenyl(s); a dibenzofuranyl; a dibenzothiphenyl; a carbazolyl substituted with a phenyl(s); a dibenzocarbazolyl; a phenanthrooxazolyl substituted with a phenyl(s); a (23-membered)heteroaryl; and a triphenylsilyl, in which the substituent(s) may be further substituted with deuterium.

Herein, a ring formed by a linkage of adjacent substituents means that at least two adjacent substituents are linked to or fused with each other to form a substituted or unsubstituted, mono- or polycyclic, (3- to 30-membered) alicyclic or aromatic ring, or the combination thereof. Preferably, the ring may be a substituted or unsubstituted, mono- or polycyclic, (3- to 26-membered) alicyclic or aromatic ring, or the combination thereof. More preferably, the ring may be a mono- or polycyclic, (5- to 25-membered) aromatic ring unsubstituted or substituted with at least one of a (C6-C18)aryl(s) and a (3- to 20-membered)heteroaryl(s). In addition, the formed ring may contain at least one heteroatom selected from B, N, O, S, Si, and P, preferably at least one heteroatom selected from N, O, and S. For example, the ring may be a benzene ring, a cyclopentane ring, an indane ring, a fluorene ring, a phenanthrene ring, an indole ring, a xanthene ring, etc.

In the present disclosure, heteroaryl, heteroarylene, and heterocycloalkyl may, each independently, contain at least one heteroatom selected from B, N, O, S, Si, and P. In addition, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, and a substituted or unsubstituted (C6-C30)aryl(3- to 30-membered)heteroarylamino.

A plurality of host materials of the present disclosure comprise a first host material and a second host material, in which the first host material comprises at least one compound represented by formula 1, and the second host material comprises at least one compound represented by formula 2. According to one embodiment of the present disclosure, the compound represented by formula 1 and the compound represented by formula 2 are different from each other.

In formula 1, ring A and ring B, each independently, represent a substituted or unsubstituted (C6-C30)arene, or a substituted or unsubstituted (3- to 30-membered)heteroarene. According to one embodiment of the present disclosure, ring A and ring B, each independently, represent a substituted or unsubstituted (C6-C25)arene, or a substituted or unsubstituted (5- to 25-membered)heteroarene. According to another embodiment of the present disclosure, ring A and ring B, each independently, represent an unsubstituted (C6-C18)arene, or an unsubstituted (5- to 20-membered)heteroarene. For example, ring A and ring B, each independently, may be a benzene ring, a naphthalene ring, a phenanthrene ring, or a pyridine ring, etc.

In formula 1, L₁ and L₂, each independently, represent a single bond, a substituted or unsubstituted (C6-C30)arylene, or a substituted or unsubstituted (3- to 30-membered)heteroarylene. According to one embodiment of the present disclosure, L₁ and L₂, each independently, represent a single bond, a substituted or unsubstituted (C6-C25)arylene, or a substituted or unsubstituted (5- to 25-membered)heteroarylene. According to another embodiment of the present disclosure, L₁ and L₂, each independently, represent a single bond, an unsubstituted (C6-C18)arylene, or an unsubstituted (5- to 20-membered)heteroarylene. For example, L₁ and L₂, each independently, may be a single bond, or a phenylene, etc.

In formula 1, Ar represents a substituted or unsubstituted (C6-C30)aryl, or a substituted or unsubstituted (3- to 30-membered)heteroaryl. According to one embodiment of the present disclosure, Ar represents a substituted or unsubstituted (C6-C25)aryl, or a substituted or unsubstituted (5- to 25-membered)heteroaryl. According to another embodiment of the present disclosure, Ar represents a (C6-C18)aryl unsubstituted or substituted with at least one of deuterium and a cyano(s): or a (5- to 20-membered)heteroaryl unsubstituted or substituted with at least one of deuterium, a cyano(s), and a (CM-C18)aryl(s). For example, Ar may be a phenyl, a naphthyl, a biphenyl, a dibenzofuranyl, a dibenzothiophenyl, or a carbazolyl substituted with a phenyl(s), etc., which may be further substituted with at least one of deuterium and a cyano(s).

In formula 1, HAr represents a substituted or unsubstituted (3- to 20-membered)heteroaryl containing a nitrogen atom(s). According to one embodiment of the present disclosure, HAr represents a substituted or unsubstituted (5- to 18-membered)heteroaryl containing a nitrogen atom(s). According to another embodiment of the present disclosure, HAr represents a substituted (5- to 18-membered)heteroaryl containing a nitrogen atom(s). Specifically, HAr may be a substituted or unsubstituted, pyridyl, triazinyl, pyrimidinyl, quinolyl, quinazolinyl, quinoxalinyl, benzoquinazolinyl, benzoquinoxalinyl, benzofuropyrimidinyl, carbazolyl, dibenzothiophenyl, benzothiophenyl, dibenzofuranyl, benzofuranyl, naphthyridinyl, benzonaphthofuranyl, or benzonaphthothiophenyl. For example, HAr may be a substituted triazinyl, or a substituted pyrimidinyl, etc. The substituent(s) of the substituted triazinyl or the substituted pyrimidinyl, each independently, may be at least one, preferably two, selected from the group consisting of a phenyl unsubstituted or substituted with a dibenzofuranyl(s); a naphthyl unsubstituted or substituted with a dibenzofuranyl(s); a biphenyl unsubstituted or substituted with a dibenzofuranyl(s); a fluorenyl unsubstituted or substituted with at least one of a methyl(s), a phenyl(s), and a naphthyl(s); a phenanthrenyl; a chrysenyl; a dibenzofuranyl; a dibenzothiophenyl; a phenanthrooxazolyl substituted with a phenyl(s); a carbazolyl substituted with a phenyl(s); a dibenzocarbazolyl; and a (23-membered)heteroaryl containing a nitrogen atom(s), in which the substituent(s) may be further substituted with at least one of deuterium and a cyano(s).

According to one embodiment of the present disclosure, the formula 1 may be represented by any one of the following formulas.

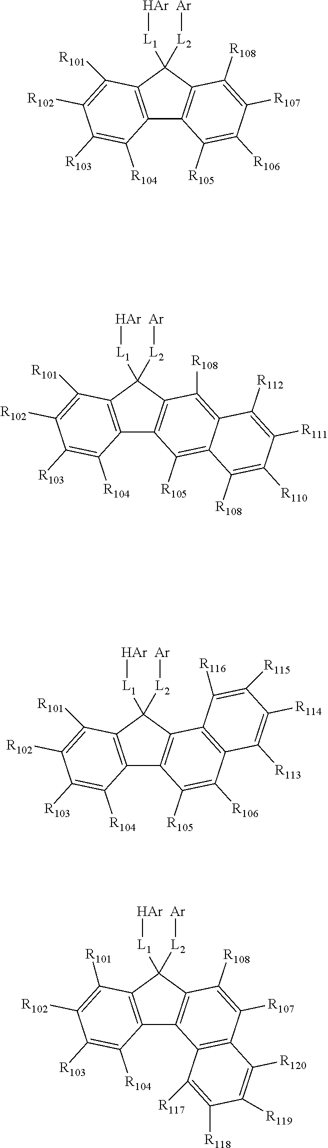

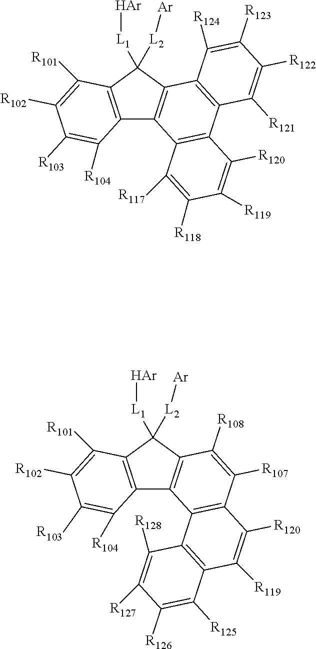

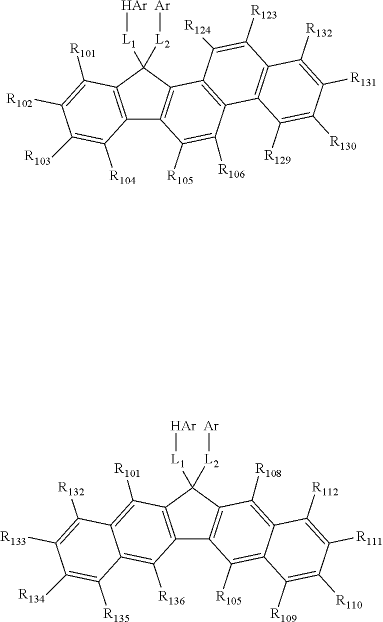

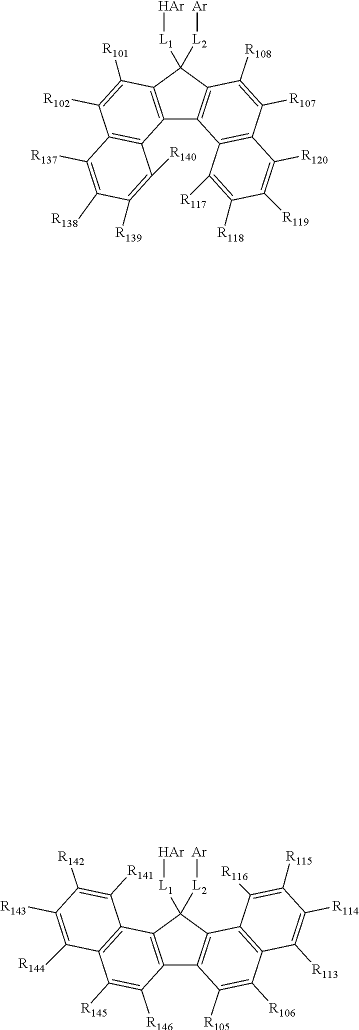

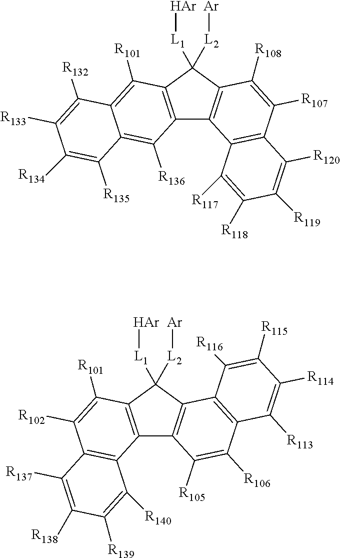

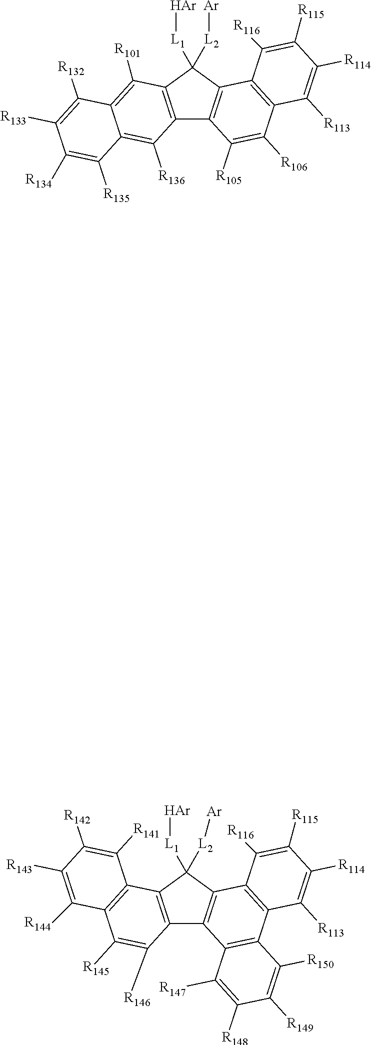

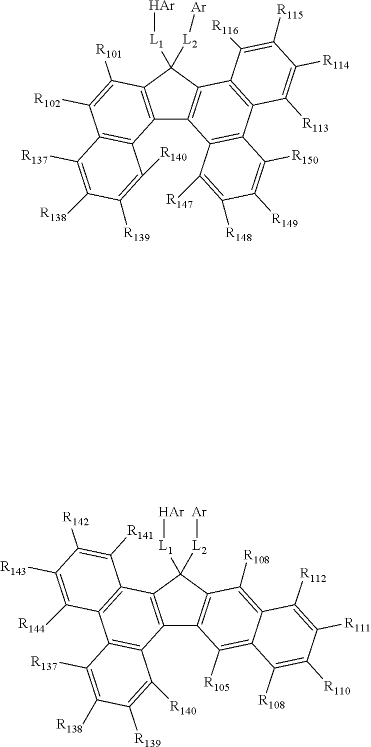

In the formulas above, R₁₀₁ to R₁₅₀, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or -L-NR₁R₂. For example, R₁₀₁ to R₁₅₀ may be hydrogen.

In the formulas above, HAr, Ar, L₁, L₂, L, R₁, and R₂ are as defined in formula 1.

In formula 2, X₂₁ and Y₂₁, each independently, represent —N═, —NR₃₁—, —O—, or —S—, with the proviso that any one of X₂₁ and Y₂₁ represents —N═ and the other one of X₂₁ and Y₂₁ represents —NR₃₁—, —O—, or —S—. According to one embodiment of the present disclosure, X₂₁ and Y₂₁, each independently, represent —N═, —O—, or —S—, with the proviso that any one of X₂₁ and Y₂₁ represents —N═ and the other one of X₂₁ and Y₂₁ represents —O—, or —S—.

R₃₁ represents a substituted or unsubstituted (C6-C30)aryl, or a substituted or unsubstituted (3- to 30-membered)heteroaryl.

In formula 2, R₂₁ represents a substituted or unsubstituted (C6-C30)aryl, or a substituted or unsubstituted (3- to 30-membered)heteroaryl. According to one embodiment of the present disclosure, R₂₁ represents a substituted or unsubstituted (C6-C25)aryl, or a substituted or unsubstituted (5- to 25-membered)heteroaryl. According to another embodiment of the present disclosure, R₂₁ represents an unsubstituted (C6-C18)aryl, or an unsubstituted (5- to 20-membered)heteroaryl. For example, R₂₁ may be a phenyl, a naphthyl, a biphenyl, a pyridyl, a quinolyl, or isoquinolyl, etc.

In formula 2, R₂ to R₂₉, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), -L-NR₁R₂, or -L₂₁-Ar₂₁; or may be linked to an adjacent substituent to form a ring(s); with the proviso that at least one of R₂₂ to R₂₉ represents -L₂₁-Ar₂₁. According to one embodiment of the present disclosure, R₂ to R₂₉, each independently, represent hydrogen, a substituted or unsubstituted (C6-C25)aryl, a substituted or unsubstituted (5- to 25-membered)heteroaryl, or -L₂₁-Ar₂₁; with the proviso that at least one of R₂₂ to R₂₉ represents -L₂₁-Ar₂₁. According to another embodiment of the present disclosure, R₂ to R₂₉, each independently, represent hydrogen or -L₂₁-Ar₂₁; with the proviso that at least one of R₂ to R₂₉ represents -L₂₁-Ar₂₁. For example, any one of R₂ to R₂ may be -L₂₁-Ar₂₁, and the others may be hydrogen.

L₂₁, each independently, represents a single bond, a substituted or unsubstituted (C6-C30)arylene, or a substituted or unsubstituted (3- to 30-membered)heteroarylene. According to one embodiment of the present disclosure, L₂₁, each independently, represents a single bond, or a substituted or unsubstituted (C6-C25)arylene. According to another embodiment of the present disclosure, L₂₁, each independently, represents a single bond, or an unsubstituted (C6-C18)arylene. For example, L₂₁, each independently, may be a single bond, a phenylene, or a naphthylene, etc.

Ar₂₁, each independently, represents a substituted or unsubstituted fused ring group of a (C3-C30) aliphatic ring(s) and a (C6-C30) aromatic ring(s), a substituted or unsubstituted (C6-C30)aryl, a substituted or unsubstituted (3- to 30-membered)heteroaryl, or —NR₃₂R₃₃. According to one embodiment of the present disclosure, Ar₂₁, each independently, represents a substituted or unsubstituted fused ring group of a (C3-C25) aliphatic ring(s) and a (C6-C18) aromatic ring(s), a substituted or unsubstituted (C6-C30)aryl, a substituted or unsubstituted (5- to 25-membered)heteroaryl, or —NR₃₂R₃₃. According to another embodiment of the present disclosure, Ar₂₁, each independently, represents an unsubstituted fused ring group of a (C3-C18) aliphatic ring(s) and a (C6-C18) aromatic ring(s), a (C6-C30)aryl unsubstituted or substituted with a (C1-C10)alkyl(s), or —NR₃₂R₃₃.

R₃₂ and R₃₃, each independently, represent a substituted or unsubstituted (C1-C30)alkyl, a substituted or unsubstituted (C2-C30)alkenyl, a substituted or unsubstituted (C6-C30)aryl, a substituted or unsubstituted (3- to 30-membered)heteroaryl, or a substituted or unsubstituted fused ring group of a (C3-C30) aliphatic ring(s) and a (C6-C30) aromatic ring(s). According to one embodiment of the present disclosure, R₃₂ and R₃₃, each independently, represent a substituted or unsubstituted (C6-C25)aryl, or a substituted or unsubstituted (5- to 25-membered)heteroaryl. According to another embodiment of the present disclosure. R₃2 and R₃₃, each independently, represent a (C6-C25)aryl unsubstituted or substituted with a (C1-C6)alkyl(s), or a (5- to 20-membered)heteroaryl unsubstituted or substituted with a (C6-C18)aryl(s). For example, R₃₂ and R₃₃, each independently, may be a phenyl, a naphthyl, a biphenyl, a dimethylfluorenyl, a diphenylfluorenyl, a phenanthrenyl, a naphthylphenyl, a phenylnaphthyl, a dimethylbenzofluorenyl, a terphenyl, a dibenzofuranyl unsubstituted or substituted with a phenyl(s), a dibenzothiophenyl unsubstituted or substituted with a phenyl(s), or a benzonaphthofuranyl, etc.

L represents a single bond, a substituted or unsubstituted (C6-C30)arylene, a substituted or unsubstituted (3- to 30-membered)heteroarylene, a substituted or unsubstituted divalent (C2-C30) aliphatic hydrocarbon group, or a substituted or unsubstituted divalent fused ring group of a (C3-C30) aliphatic ring(s) and a (C6-C30) aromatic ring(s).

R₁ and R₂, each independently, represent hydrogen, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl.

Specifically, Ar₂₁ may be a substituted or unsubstituted phenyl, a substituted or unsubstituted naphthyl, a substituted or unsubstituted o-biphenyl, a substituted or unsubstituted m-biphenyl, a substituted or unsubstituted p-biphenyl, a substituted or unsubstituted o-terphenyl, a substituted or unsubstituted m-terphenyl, a substituted or unsubstituted p-terphenyl, a substituted or unsubstituted triphenylenyl, a substituted or unsubstituted phenanthrenyl, a substituted or unsubstituted benzo[c]phenanthrenyl, a substituted or unsubstituted chrysenyl, a substituted or unsubstituted fluoranthenyl, a substituted or unsubstituted fluorenyl, a substituted or unsubstituted benzofluorenyl, a substituted or unsubstituted spirobifluorenyl, a substituted or unsubstituted spiro[cyclopentane-fluoren]yl, a substituted or unsubstituted spiro[dihydroindene-fluoren]yl, a substituted or unsubstituted spiro[benzofluorene-fluoren]yl, a substituted or unsubstituted carbazolyl, a substituted or unsubstituted benzocarbazolyl, a substituted or unsubstituted dibenzocarbazolyl, a substituted or unsubstituted dibenzothiophenyl, a substituted or unsubstituted benzothiophenyl, a substituted or unsubstituted benzonaphthothiophenyl, a substituted or unsubstituted dibenzofuranyl, a substituted or unsubstituted benzofuranyl, or a substituted or unsubstituted benzonaphthofuranyl; or may be an amino substituted with at least one selected from the group consisting of a phenyl, a naphthyl, a naphthylphenyl, a phenylnaphthyl, an o-biphenyl, an m-biphenyl, a p-biphenyl, an o-terphenyl, an m-terphenyl, a p-terphenyl, a dimethylfluorenyl, a diphenylfluorenyl, a dimethylbenzofluorenyl, a phenanthrenyl, a dibenzothiophenyl unsubstituted or substituted with a phenyl(s), a benzonaphthofuranyl, and a dibenzofuranyl unsubstituted or substituted with a phenyl(s).

According to one embodiment of the present disclosure, the formula 2 may be represented by any one of the following formulas.

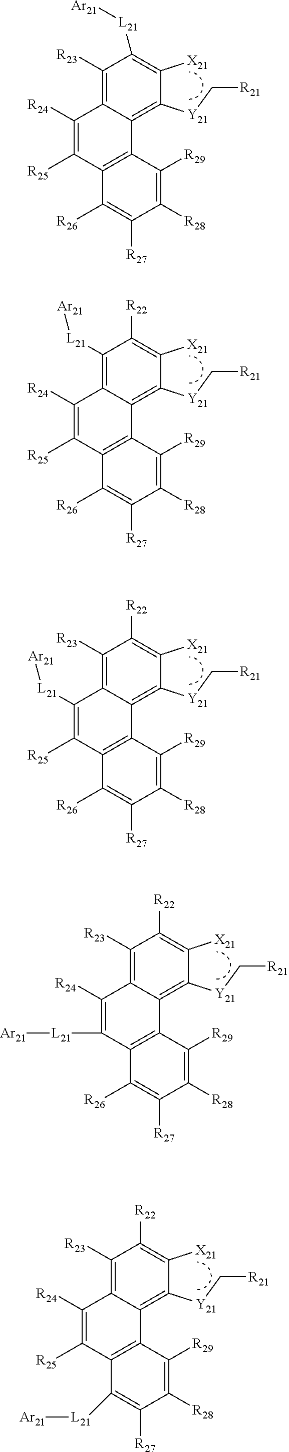

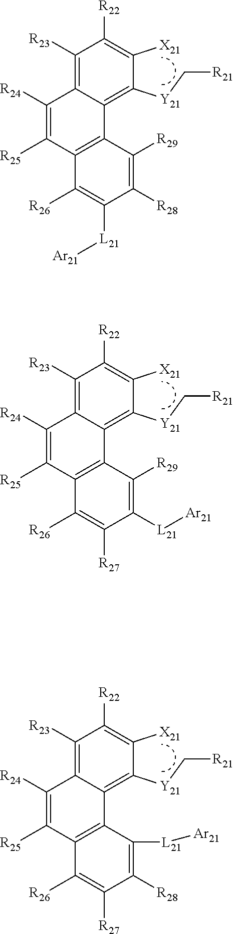

In the formulas above, X₂₁, Y₂₁, L₂₁, Ar₂₁, and R₂₁ to R₂₉ are as defined in formula 2.

In formula 3, A₁ and A₂, each independently, represent a substituted or unsubstituted (C6-C30)aryl, a substituted or unsubstituted dibenzofuranyl, a substituted or unsubstituted dibenzothiophenyl, or a substituted or unsubstituted carbazolyl. According to one embodiment of the present disclosure, A₁ and A₂, each independently, represent a substituted or unsubstituted (C6-C25)aryl, a substituted or unsubstituted dibenzofuranyl, a substituted or unsubstituted dibenzothiophenyl, or a substituted or unsubstituted carbazolyl. According to another embodiment of the present disclosure, A₁ and A₂, each independently, represent a (C6-C25)aryl unsubstituted or substituted with at least one of deuterium, a (C1-C6)alkyl(s), a (C6-C25)aryl(s), a (5- to 20-membered)heteroaryl(s), and a tri(C6-C18)arylsilyl(s); a dibenzofuranyl unsubstituted or substituted with at least one of deuterium and a (C6-C18)aryl(s); a dibenzothiophenyl unsubstituted or substituted with at least one of deuterium and a (C6-C18)aryl(s); or a carbazolyl unsubstituted or substituted with at least one of deuterium and a (C6-C18)aryl(s). For example, A₁ and A₂, each independently, may be a phenyl unsubstituted or substituted with deuterium, a methyl(s), a pyridyl unsubstituted or substituted with a phenyl(s), a dibenzofuranyl(s), a dibenzothiophenyl(s), or triphenylsilyl(s); a naphthyl; a biphenyl; a phenylnaphthyl; a naphthylphenyl; a terphenyl; a triphenylenyl; a phenyl substituted with a triphenylenyl(s); a dimethylfluorenyl; a diphenylfluorenyl; a dimethylbenzofluorenyl; a dibenzofuranyl unsubstituted or substituted with a phenyl(s); a dibenzothiophenyl unsubstituted or substituted with a phenyl(s); or a carbazolyl unsubstituted or substituted with a phenyl(s) or a naphthyl(s), which may be further substituted with deuterium.

In formula 3, one of X₁₅ to X₁₆ and one of X₁₉ to X₂₂ are linked to each other to form a single bond. The remaining X₁₅ to X₂₂ which do not form a single bond, X₁₁ to X₁₄, and X₂₃ to X₂₆, each independently, represent hydrogen, deuterium, a substituted or unsubstituted (C6-C30)aryl, or a substituted or unsubstituted (3- to 30-membered)heteroaryl; or may be linked to an adjacent substituent to form a ring(s). According to one embodiment, the remaining X₁₅ to X₂₂ which do not form a single bond, X₁₁ to X₁₄, and X₂₃ to X₂₆, each independently, represent hydrogen, deuterium, or a (5- to 20-membered)heteroaryl unsubstituted or substituted with deuterium; or may be linked to an adjacent substituent to form a substituted or unsubstituted, mono- or polycyclic, (3- to 30-membered) alicyclic or aromatic ring, or the combination thereof. For example, the remaining X₁₅ to X₂₂ which do not form a single bond, X₁₁ to X₁₄, and X₂₃ to X₂₆, each independently, may be hydrogen, deuterium, or a dibenzothiophenyl unsubstituted or substituted with deuterium, or a dibenzofuranyl unsubstituted or substituted with deuterium; or may be linked to an adjacent substituent to form a benzene ring unsubstituted or substituted with deuterium.

The compound represented by formula 1 may be at least one selected from the following compounds, but is not limited thereto.

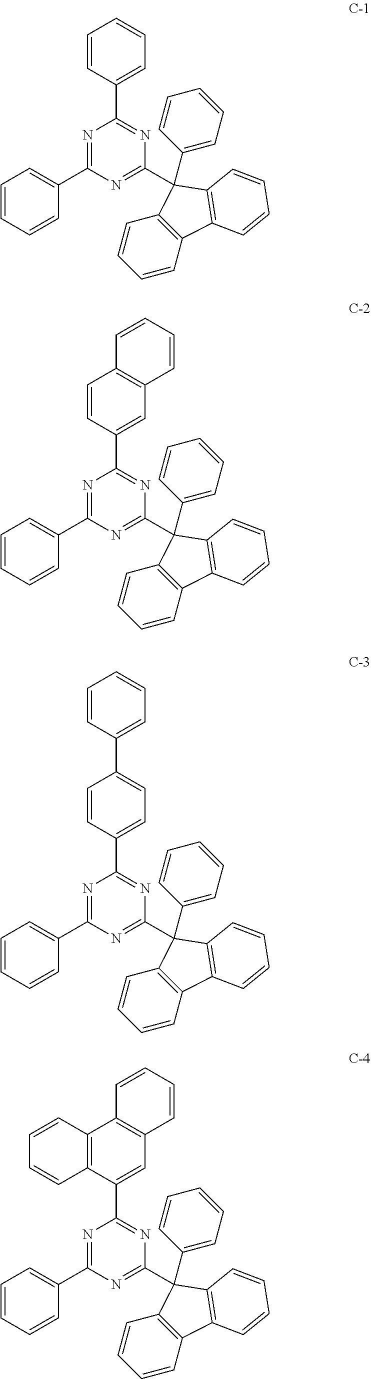

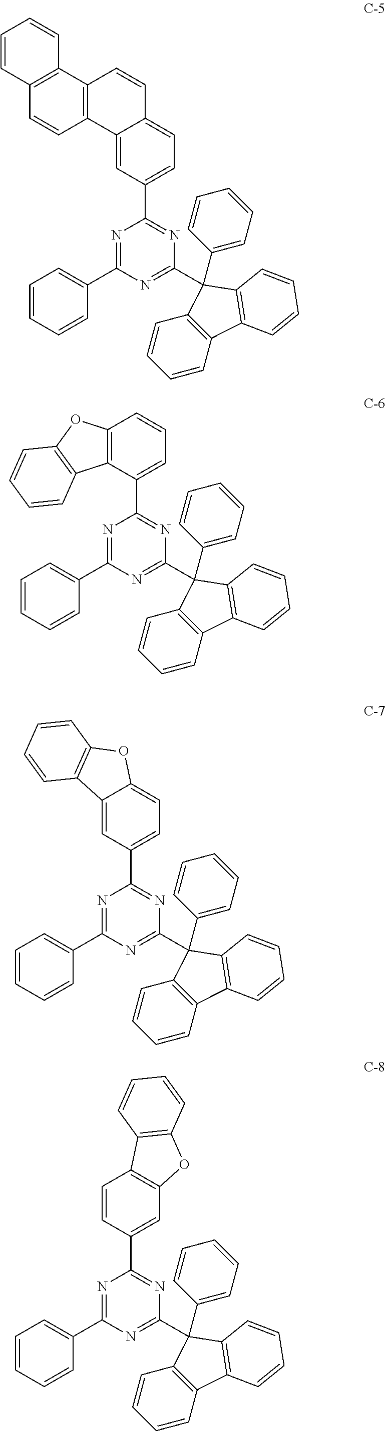

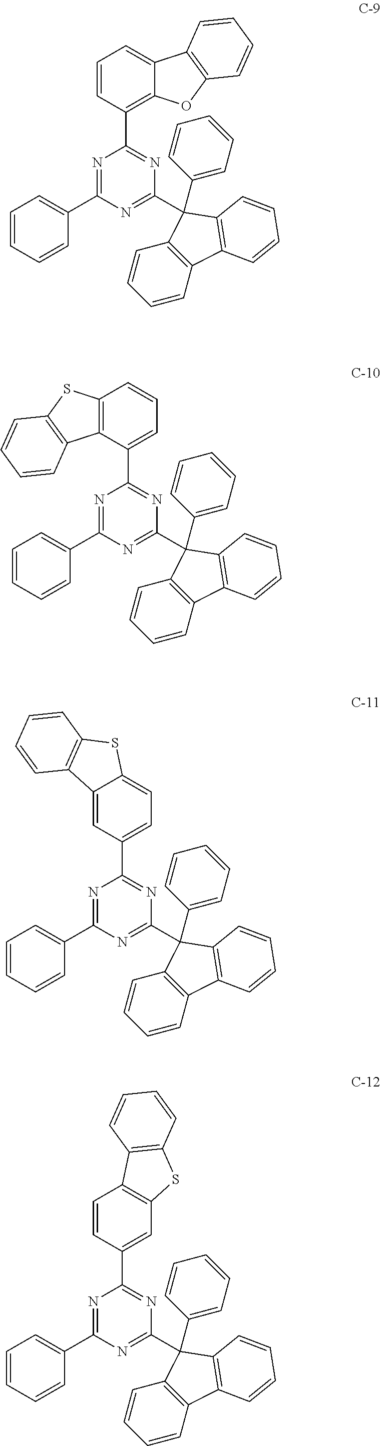 [SMILES: c1ccc(-c2nc(-c3cccc4c3oc3ccccc34)nc(C3(c4ccccc4)c4ccccc4-c4ccccc43)n2)cc1 ; c1ccc(-c2nc(-c3cccc4sc5ccccc5c34)nc(C3(c4ccccc4)c4ccccc4-c4ccccc43)n2)cc1 ; c1ccc(-c2nc(-c3ccc4sc5ccccc5c4c3)nc(C3(c4ccccc4)c4ccccc4-c4ccccc43)n2)cc1 ; c1ccc(-c2nc(-c3ccc4c(c3)sc3ccccc34)nc(C3(c4ccccc4)c4ccccc4-c4ccccc43)n2)cc1]

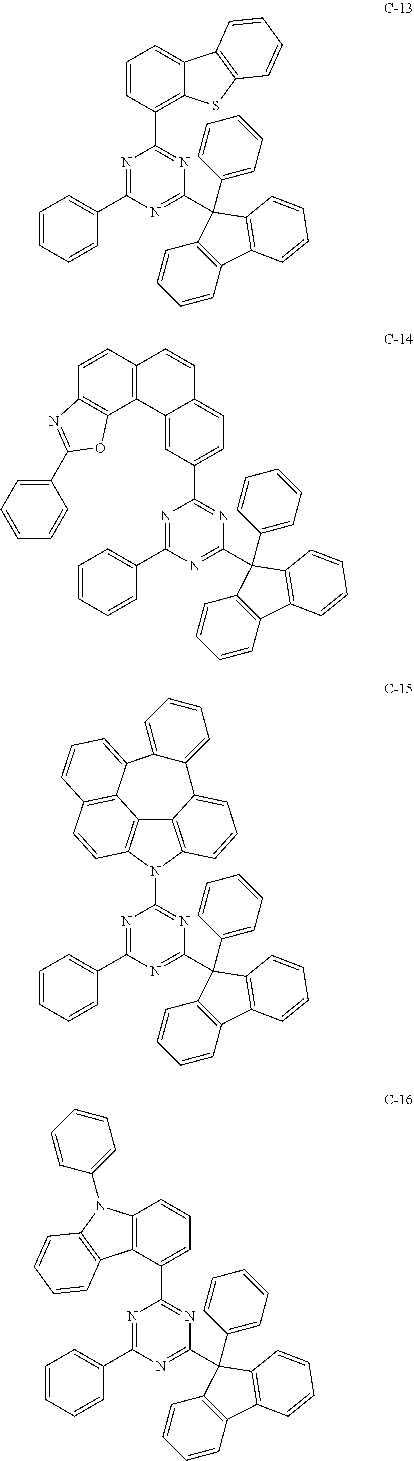

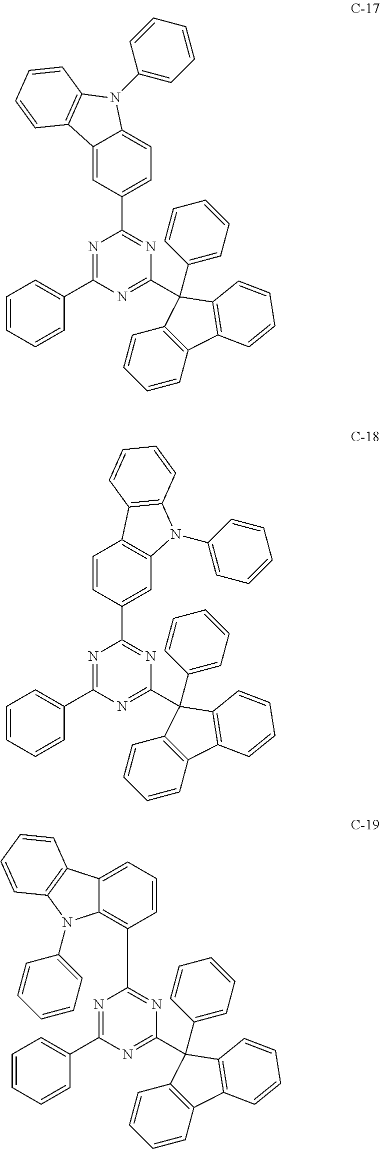 [SMILES: c1ccc(-c2nc(-c3ccc4c(c3)c3ccccc3n4-c3ccccc3)nc(C3(c4ccccc4)c4ccccc4-c4ccccc43)n2)cc1 ; c1ccc(-c2nc(-c3ccc4c5ccccc5n(-c5ccccc5)c4c3)nc(C3(c4ccccc4)c4ccccc4-c4ccccc43)n2)cc1 ; c1ccc(-c2nc(-c3cccc4c5ccccc5n(-c5ccccc5)c34)nc(C3(c4ccccc4)c4ccccc4-c4ccccc43)n2)cc1]

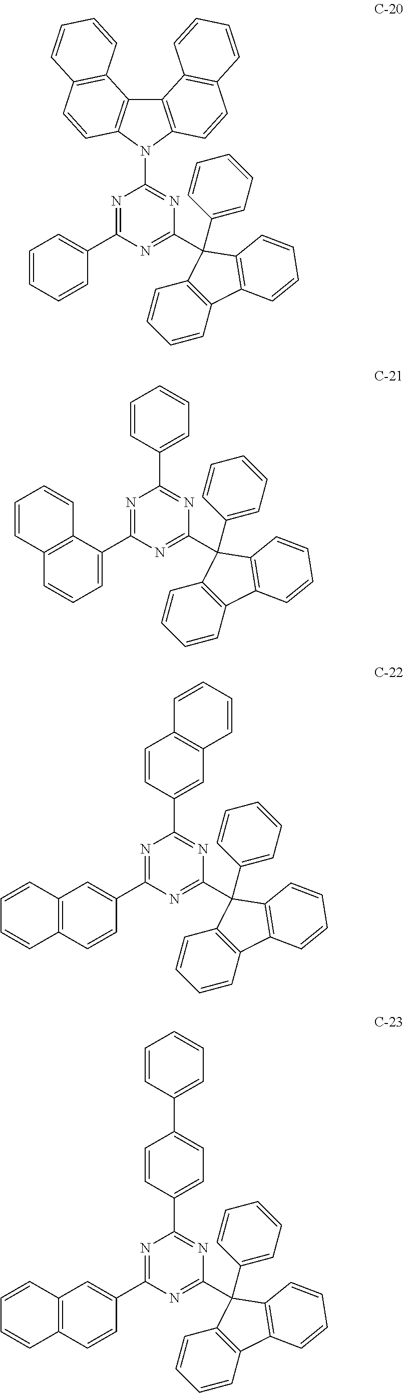

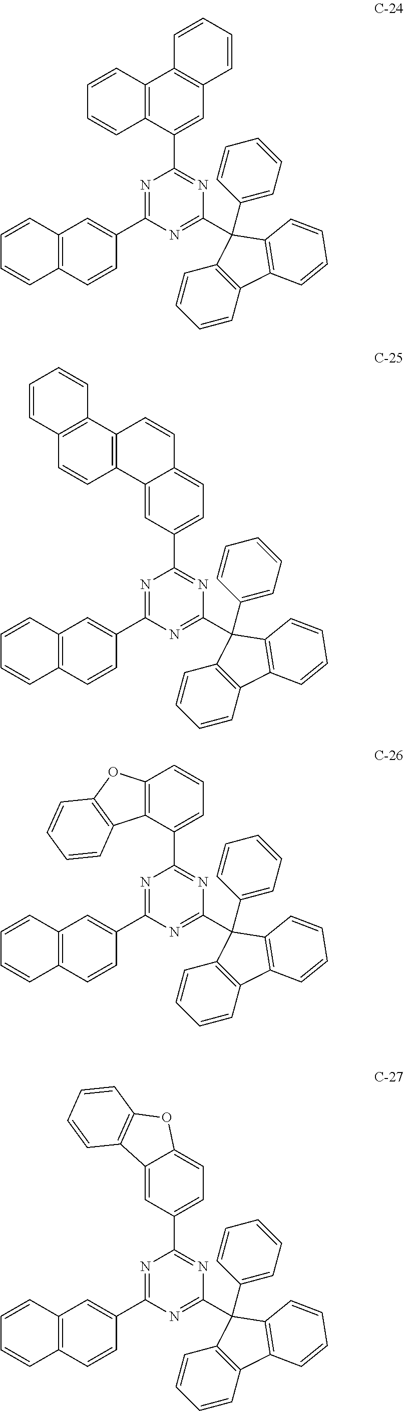

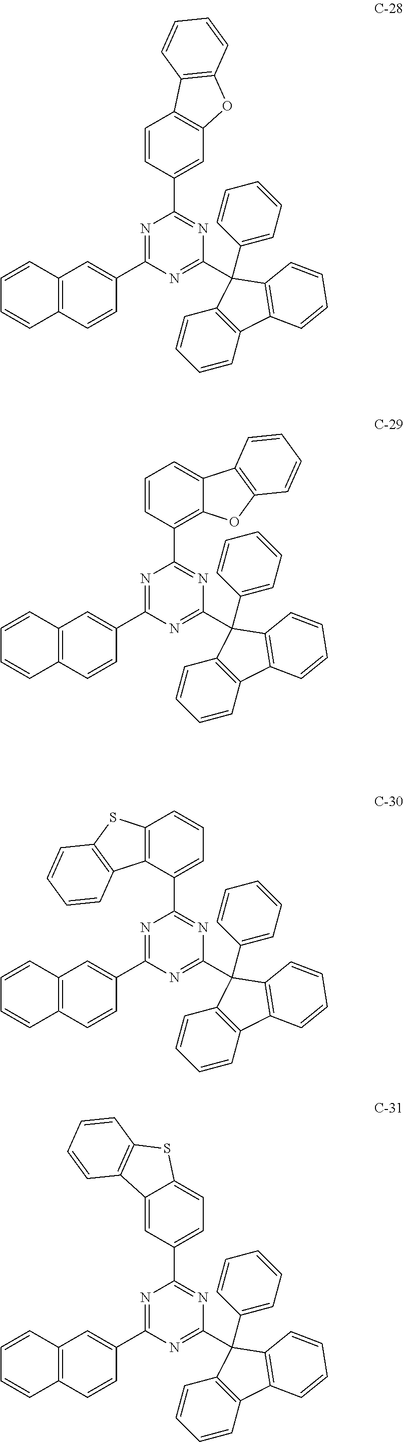 [SMILES: c1ccc(C2(c3nc(-c4ccc5ccccc5c4)nc(-c4ccc5c(c4)oc4ccccc45)n3)c3ccccc3-c3ccccc32)cc1 ; c1ccc(C2(c3nc(-c4ccc5ccccc5c4)nc(-c4cccc5c4oc4ccccc45)n3)c3ccccc3-c3ccccc32)cc1 ; c1ccc(C2(c3nc(-c4ccc5ccccc5c4)nc(-c4cccc5sc6ccccc6c45)n3)c3ccccc3-c3ccccc32)cc1 ; c1ccc(C2(c3nc(-c4ccc5ccccc5c4)nc(-c4ccc5sc6ccccc6c5c4)n3)c3ccccc3-c3ccccc32)cc1]

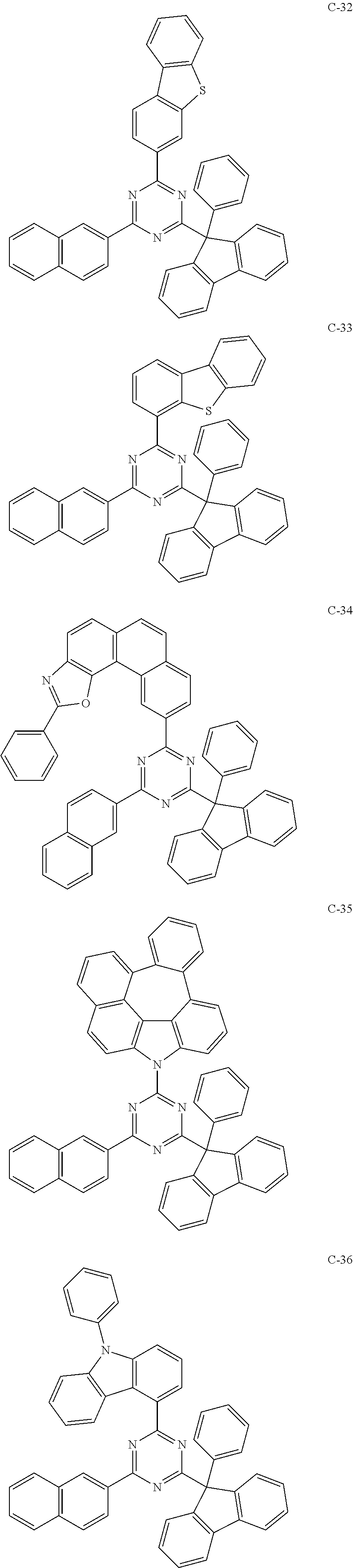

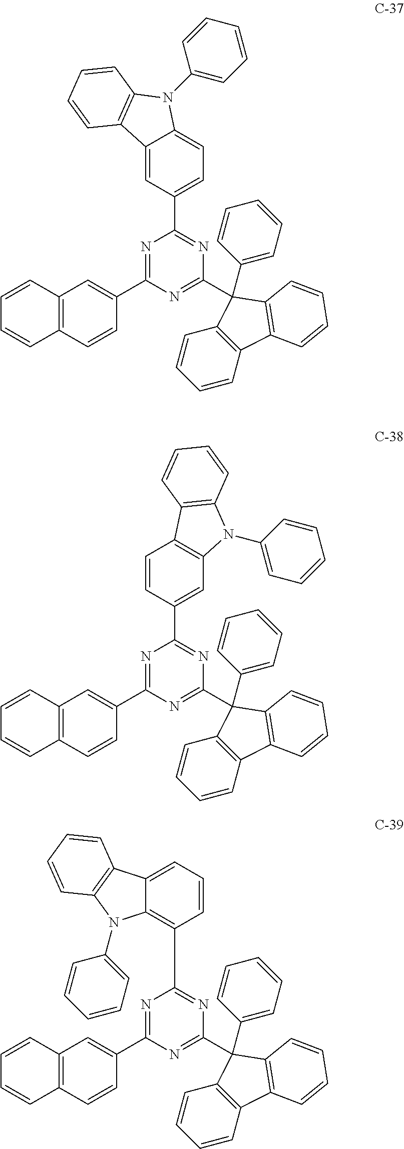 [SMILES: c1ccc(-n2c3ccccc3c3cc(-c4nc(-c5ccc6ccccc6c5)nc(C5(c6ccccc6)c6ccccc6-c6ccccc65)n4)ccc32)cc1 ; c1ccc(-n2c3ccccc3c3ccc(-c4nc(-c5ccc6ccccc6c5)nc(C5(c6ccccc6)c6ccccc6-c6ccccc65)n4)cc32)cc1 ; c1ccc(-n2c3ccccc3c3cccc(-c4nc(-c5ccc6ccccc6c5)nc(C5(c6ccccc6)c6ccccc6-c6ccccc65)n4)c32)cc1]

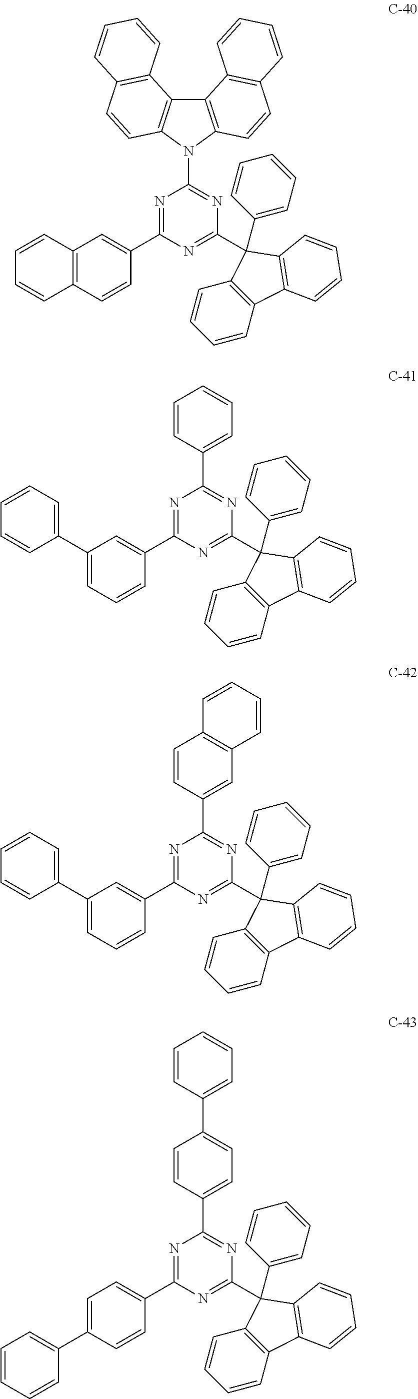

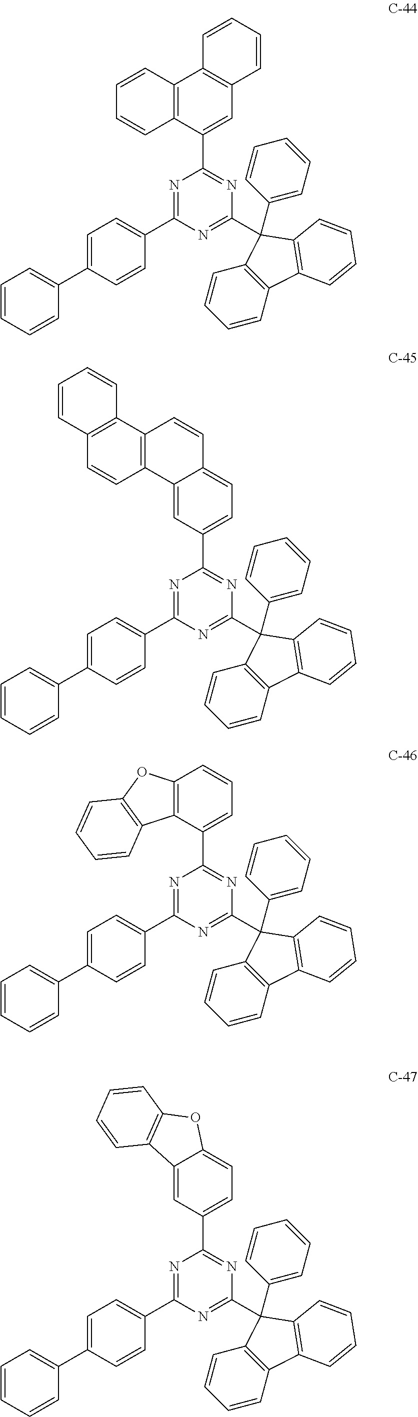

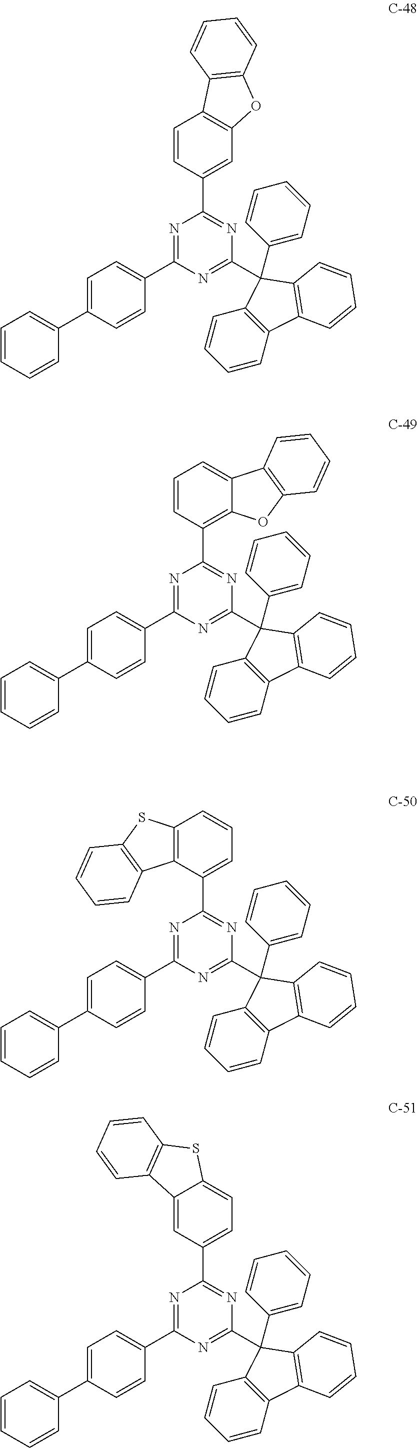 [SMILES: c1ccc(-c2ccc(-c3nc(-c4ccc5c(c4)oc4ccccc45)nc(C4(c5ccccc5)c5ccccc5-c5ccccc54)n3)cc2)cc1 ; c1ccc(-c2ccc(-c3nc(-c4cccc5c4oc4ccccc45)nc(C4(c5ccccc5)c5ccccc5-c5ccccc54)n3)cc2)cc1 ; c1ccc(-c2ccc(-c3nc(-c4cccc5sc6ccccc6c45)nc(C4(c5ccccc5)c5ccccc5-c5ccccc54)n3)cc2)cc1 ; c1ccc(-c2ccc(-c3nc(-c4ccc5sc6ccccc6c5c4)nc(C4(c5ccccc5)c5ccccc5-c5ccccc54)n3)cc2)cc1]

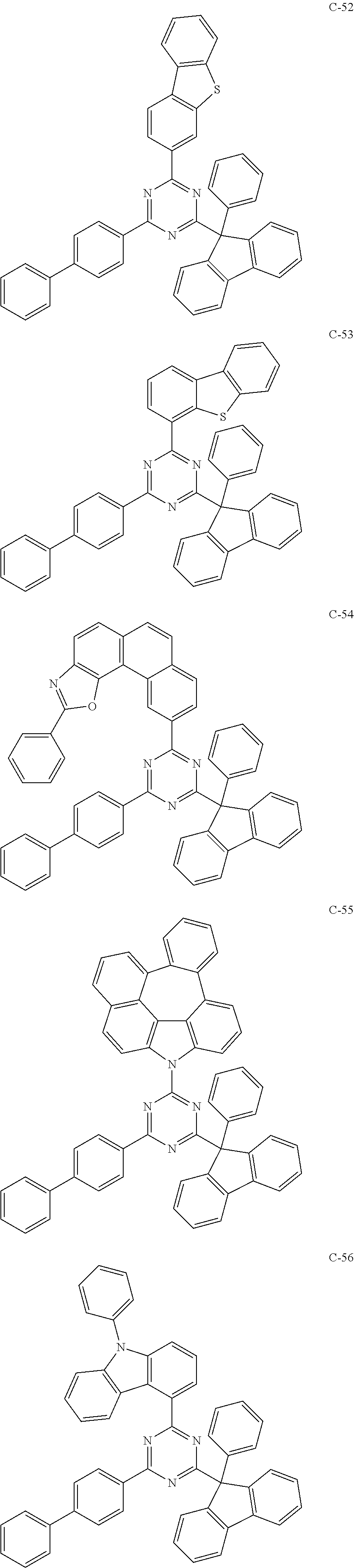

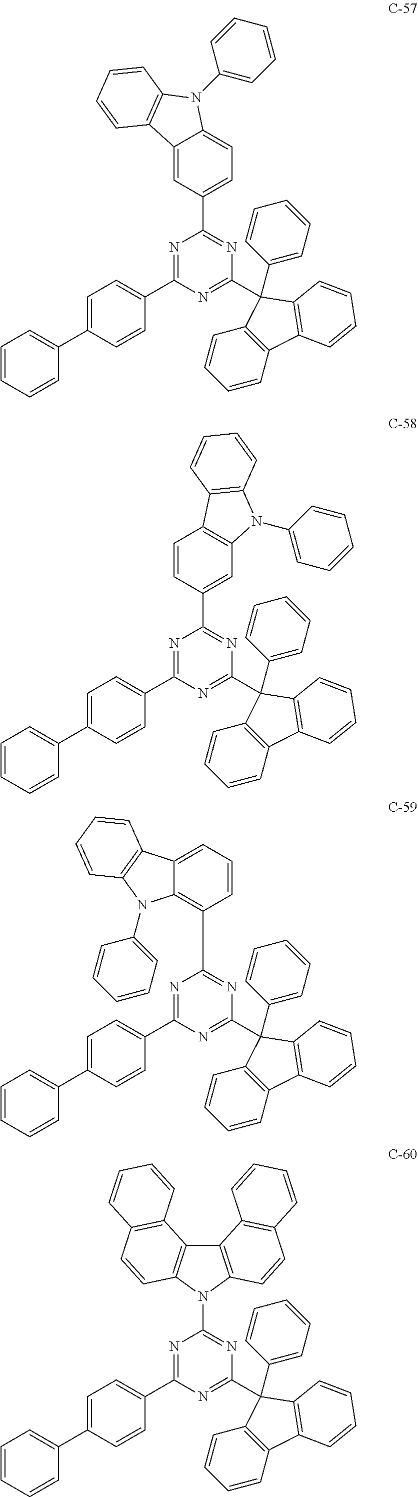

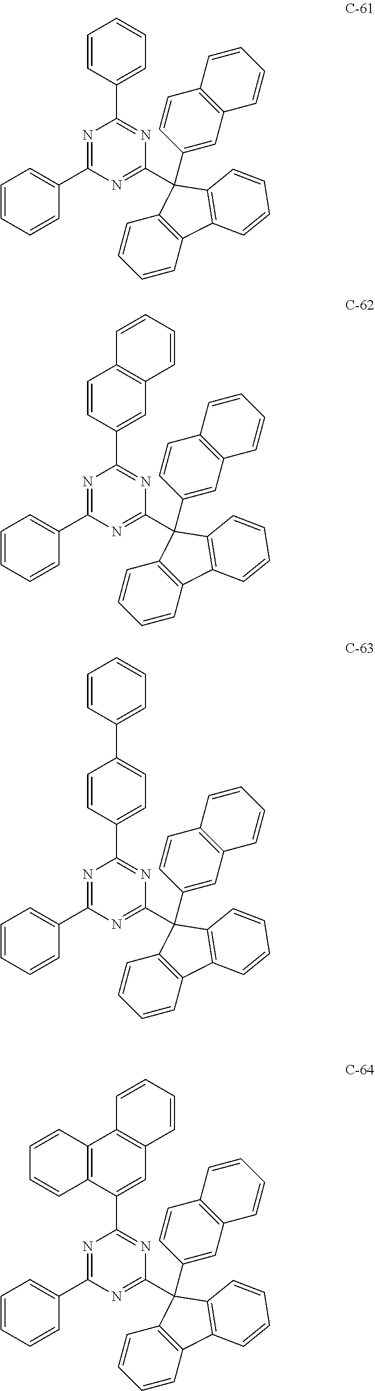

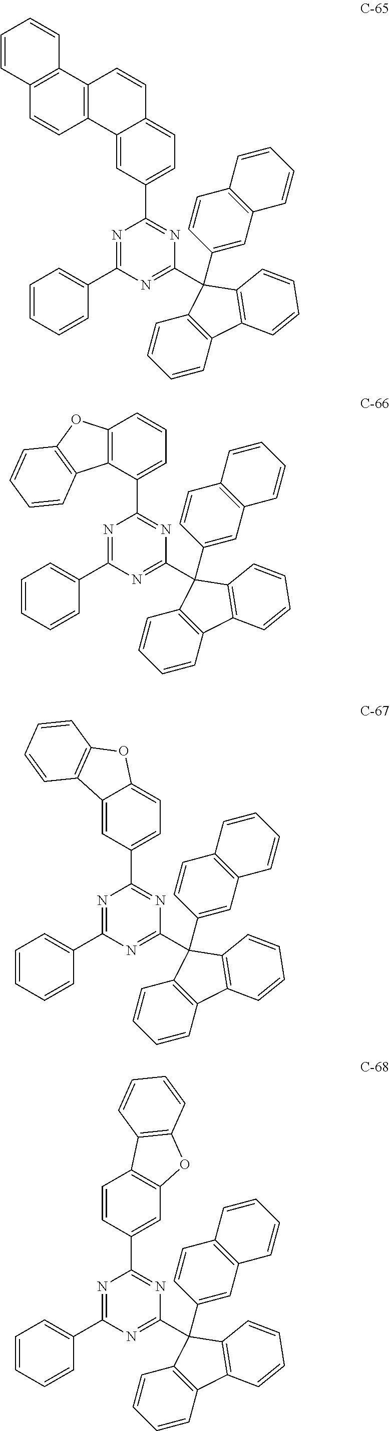

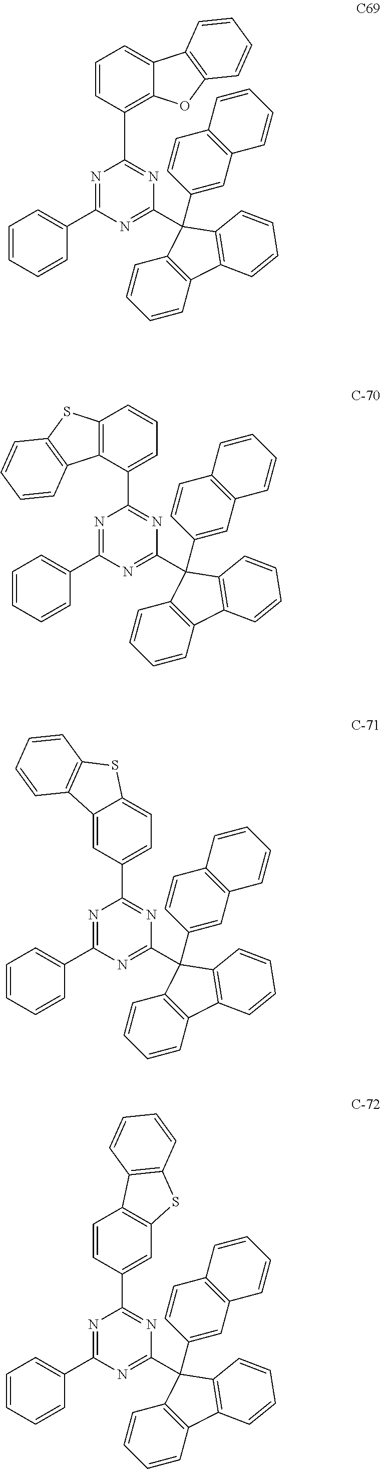 [SMILES: c1ccc(-c2nc(-c3cccc4c3oc3ccccc34)nc(C3(c4ccc5ccccc5c4)c4ccccc4-c4ccccc43)n2)cc1 ; c1ccc(-c2nc(-c3cccc4sc5ccccc5c34)nc(C3(c4ccc5ccccc5c4)c4ccccc4-c4ccccc43)n2)cc1 ; c1ccc(-c2nc(-c3ccc4sc5ccccc5c4c3)nc(C3(c4ccc5ccccc5c4)c4ccccc4-c4ccccc43)n2)cc1 ; c1ccc(-c2nc(-c3ccc4c(c3)sc3ccccc34)nc(C3(c4ccc5ccccc5c4)c4ccccc4-c4ccccc43)n2)cc1]

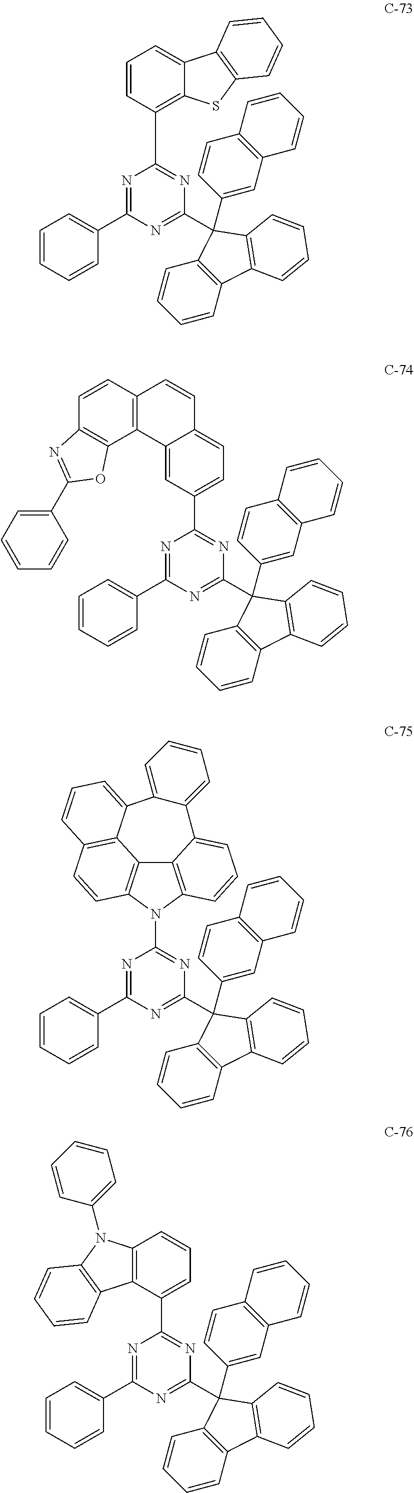 [SMILES: c1ccc(-c2nc(-c3cccc4c3sc3ccccc34)nc(C3(c4ccc5ccccc5c4)c4ccccc4-c4ccccc43)n2)cc1 ; c1ccc(-c2nc(-c3ccc4ccc5ccc6nc(-c7ccccc7)oc6c5c4c3)nc(C3(c4ccc5ccccc5c4)c4ccccc4-c4ccccc43)n2)cc1 ; c1ccc(-c2nc(-n3c4cccc5c4c4c6c(cccc6ccc43)-c3ccccc3-5)nc(C3(c4ccc5ccccc5c4)c4ccccc4-c4ccccc43)n2)cc1 ; c1ccc(-c2nc(-c3cccc4c3c3ccccc3n4-c3ccccc3)nc(C3(c4ccc5ccccc5c4)c4ccccc4-c4ccccc43)n2)cc1]

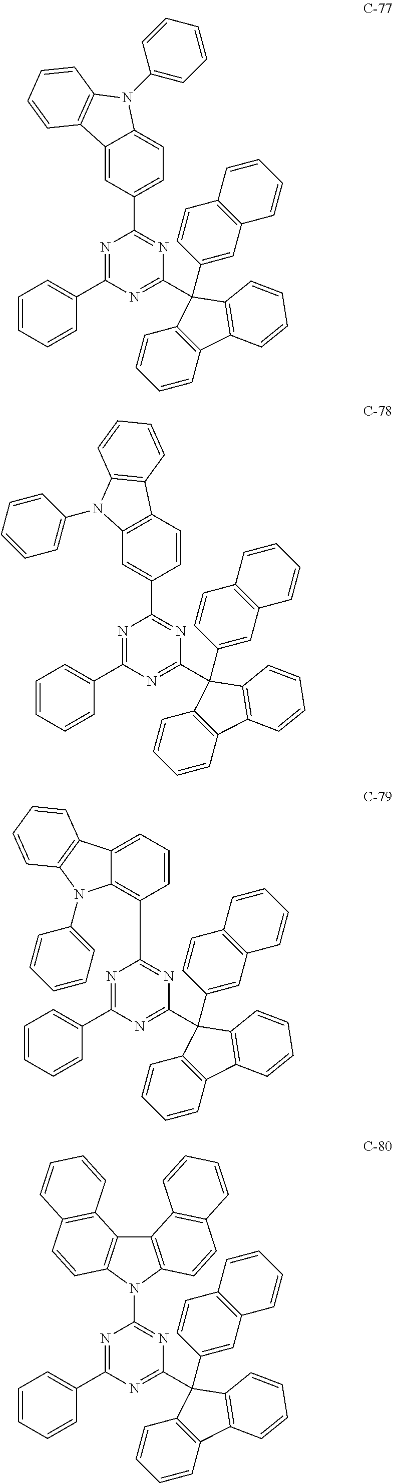

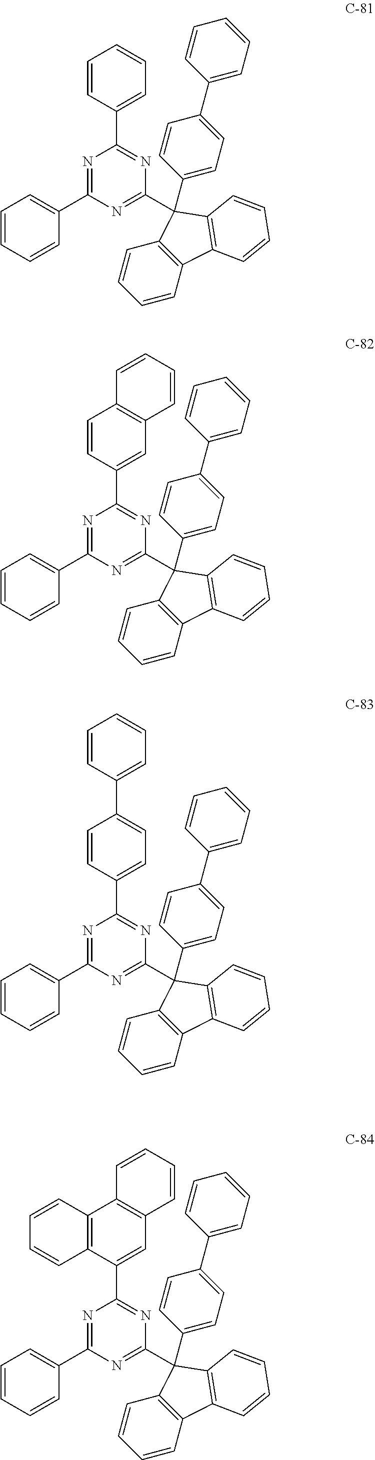 [SMILES: c1ccc(-c2ccc(C3(c4nc(-c5ccccc5)nc(-c5ccccc5)n4)c4ccccc4-c4ccccc43)cc2)cc1 ; c1ccc(-c2ccc(C3(c4nc(-c5ccccc5)nc(-c5ccc6ccccc6c5)n4)c4ccccc4-c4ccccc43)cc2)cc1 ; c1ccc(-c2ccc(-c3nc(-c4ccccc4)nc(C4(c5ccc(-c6ccccc6)cc5)c5ccccc5-c5ccccc54)n3)cc2)cc1 ; c1ccc(-c2ccc(C3(c4nc(-c5ccccc5)nc(-c5cc6ccccc6c6ccccc56)n4)c4ccccc4-c4ccccc43)cc2)cc1]

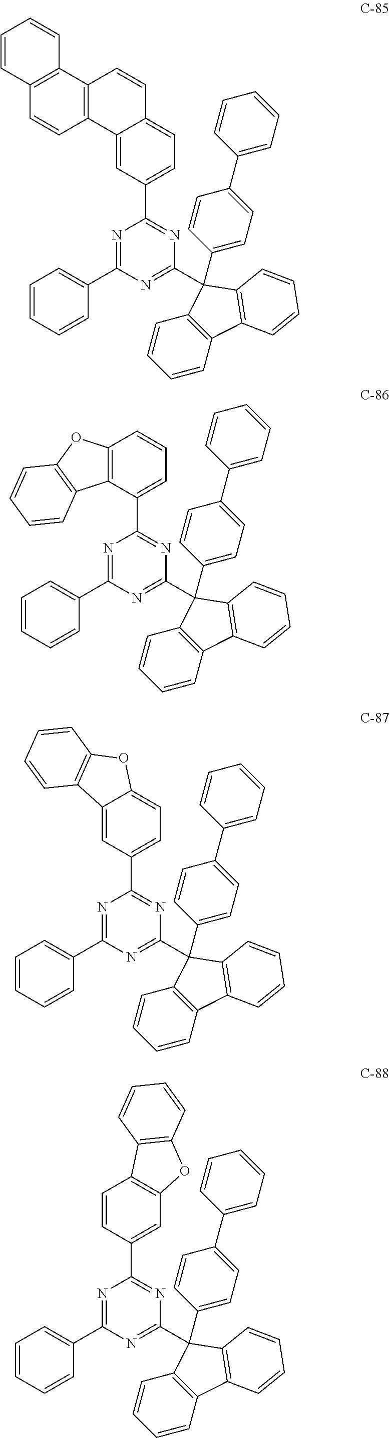

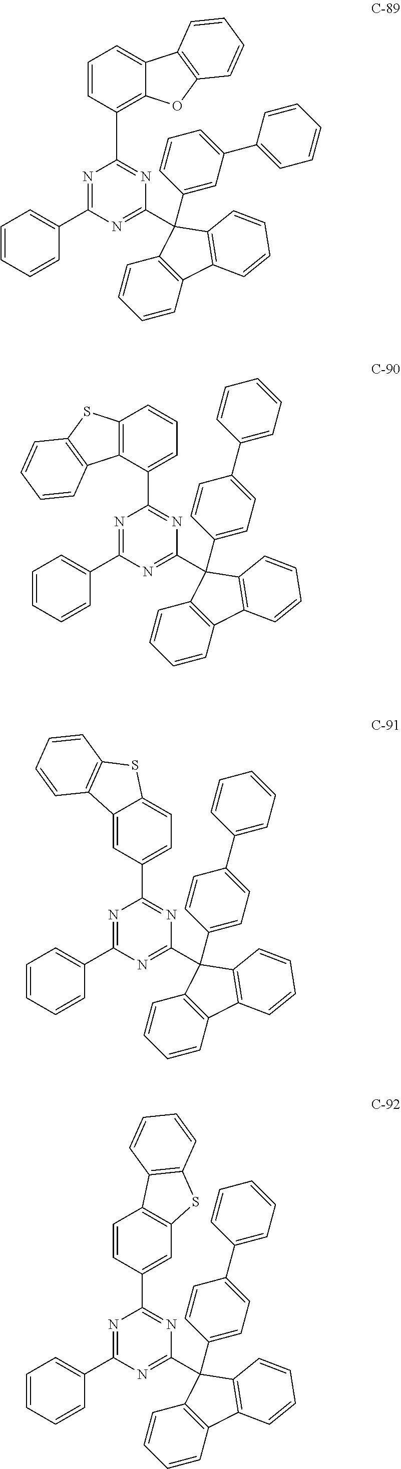 [SMILES: c1ccc(-c2cccc(C3(c4nc(-c5ccccc5)nc(-c5cccc6c5oc5ccccc56)n4)c4ccccc4-c4ccccc43)c2)cc1 ; c1ccc(-c2ccc(C3(c4nc(-c5ccccc5)nc(-c5cccc6sc7ccccc7c56)n4)c4ccccc4-c4ccccc43)cc2)cc1 ; c1ccc(-c2ccc(C3(c4nc(-c5ccccc5)nc(-c5ccc6sc7ccccc7c6c5)n4)c4ccccc4-c4ccccc43)cc2)cc1 ; c1ccc(-c2ccc(C3(c4nc(-c5ccccc5)nc(-c5ccc6c(c5)sc5ccccc56)n4)c4ccccc4-c4ccccc43)cc2)cc1]

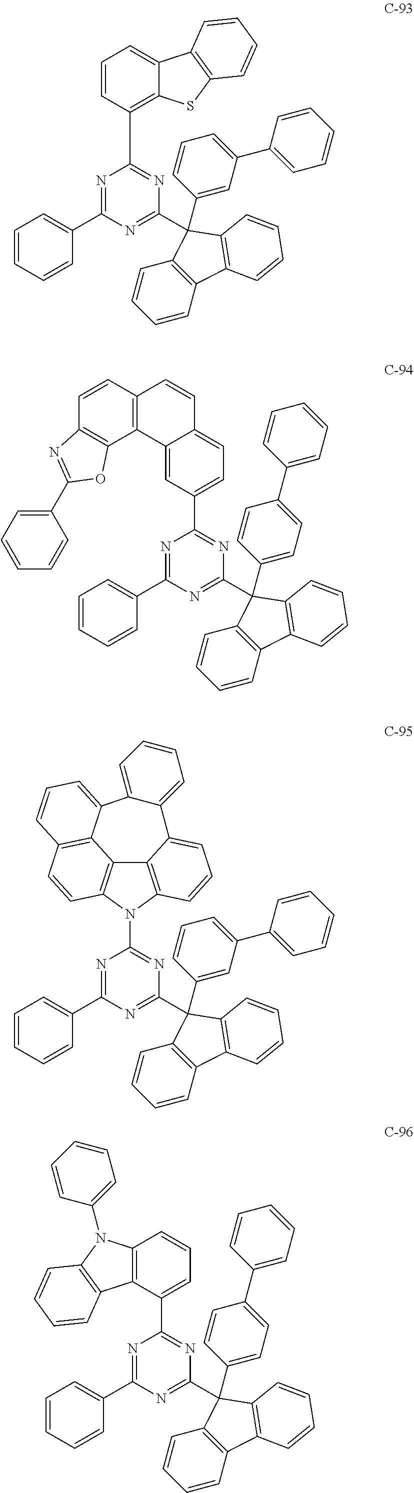 [SMILES: c1ccc(-c2cccc(C3(c4nc(-c5ccccc5)nc(-c5cccc6c5sc5ccccc56)n4)c4ccccc4-c4ccccc43)c2)cc1 ; c1ccc(-c2ccc(C3(c4nc(-c5ccccc5)nc(-c5ccc6ccc7ccc8nc(-c9ccccc9)oc8c7c6c5)n4)c4ccccc4-c4ccccc43)cc2)cc1 ; c1ccc(-c2cccc(C3(c4nc(-c5ccccc5)nc(-n5c6cccc7c6c6c8c(cccc8ccc65)-c5ccccc5-7)n4)c4ccccc4-c4ccccc43)c2)cc1 ; c1ccc(-c2ccc(C3(c4nc(-c5ccccc5)nc(-c5cccc6c5c5ccccc5n6-c5ccccc5)n4)c4ccccc4-c4ccccc43)cc2)cc1]

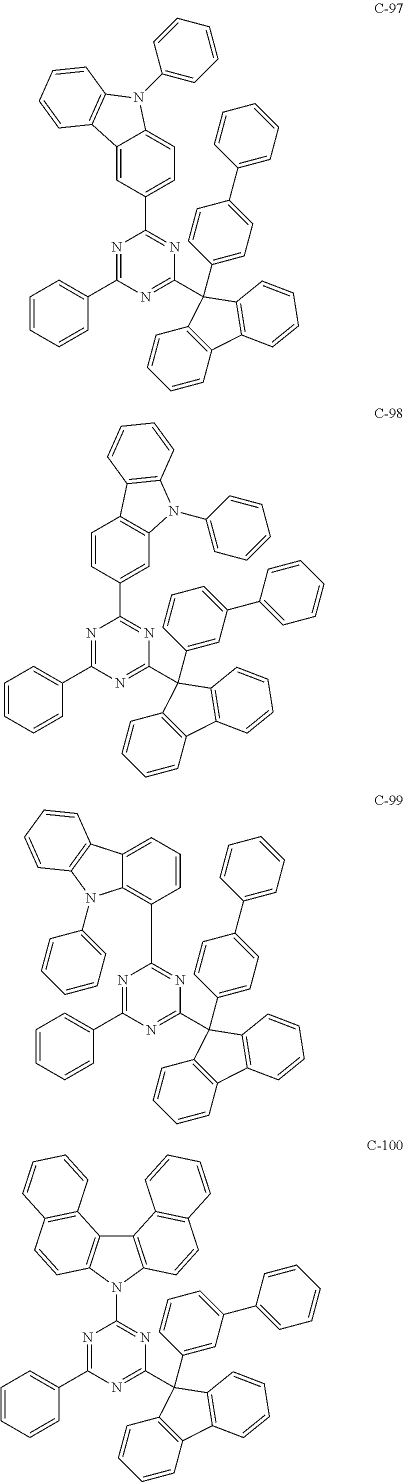

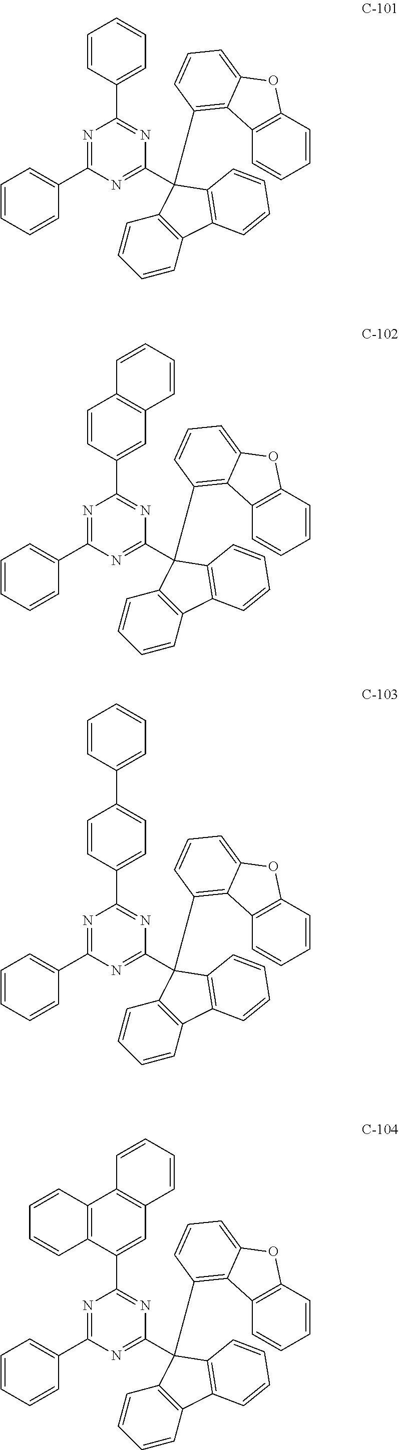 [SMILES: c1ccc(-c2nc(-c3ccccc3)nc(C3(c4cccc5oc6ccccc6c45)c4ccccc4-c4ccccc43)n2)cc1 ; c1ccc(-c2nc(-c3ccc4ccccc4c3)nc(C3(c4cccc5oc6ccccc6c45)c4ccccc4-c4ccccc43)n2)cc1 ; c1ccc(-c2ccc(-c3nc(-c4ccccc4)nc(C4(c5cccc6oc7ccccc7c56)c5ccccc5-c5ccccc54)n3)cc2)cc1 ; c1ccc(-c2nc(-c3cc4ccccc4c4ccccc34)nc(C3(c4cccc5oc6ccccc6c45)c4ccccc4-c4ccccc43)n2)cc1]

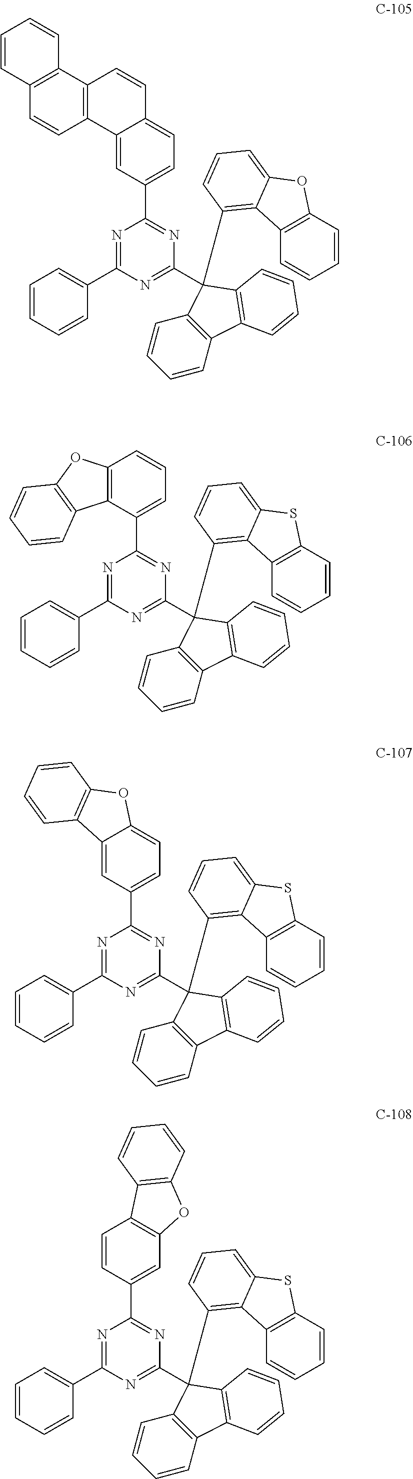

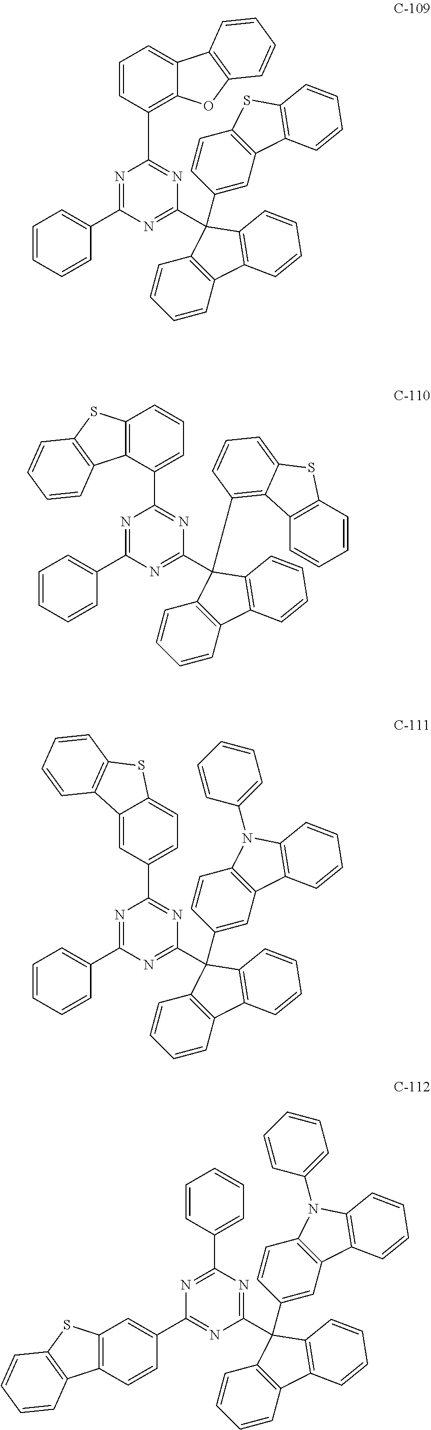 [SMILES: c1ccc(-c2nc(-c3cccc4c3oc3ccccc34)nc(C3(c4ccc5sc6ccccc6c5c4)c4ccccc4-c4ccccc43)n2)cc1 ; c1ccc(-c2nc(-c3cccc4sc5ccccc5c34)nc(C3(c4cccc5sc6ccccc6c45)c4ccccc4-c4ccccc43)n2)cc1 ; c1ccc(-c2nc(-c3ccc4sc5ccccc5c4c3)nc(C3(c4ccc5c(c4)c4ccccc4n5-c4ccccc4)c4ccccc4-c4ccccc43)n2)cc1 ; c1ccc(-c2nc(-c3ccc4c(c3)sc3ccccc34)nc(C3(c4ccc5c(c4)c4ccccc4n5-c4ccccc4)c4ccccc4-c4ccccc43)n2)cc1]

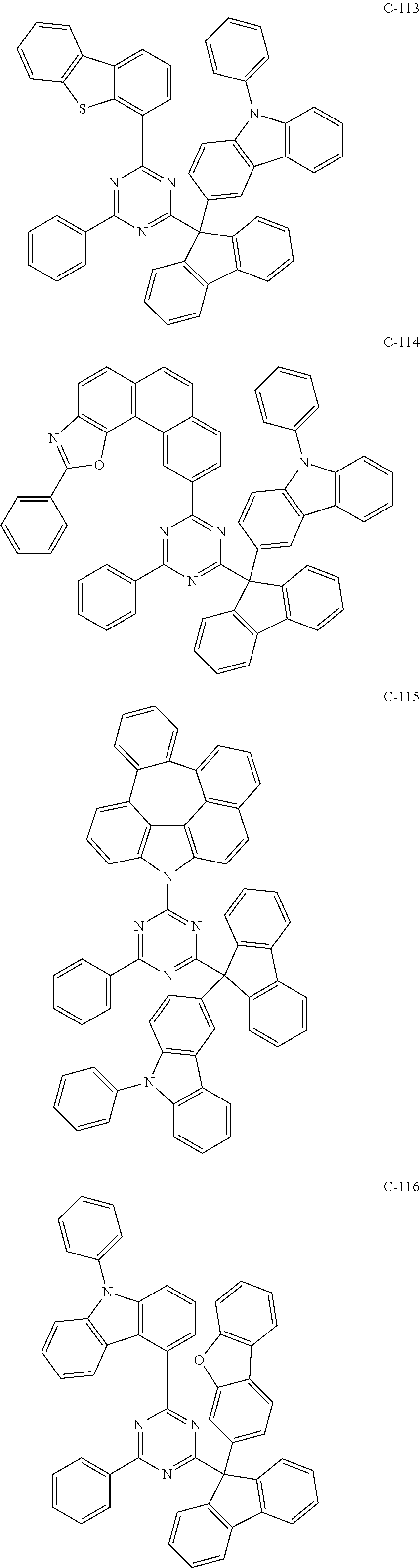

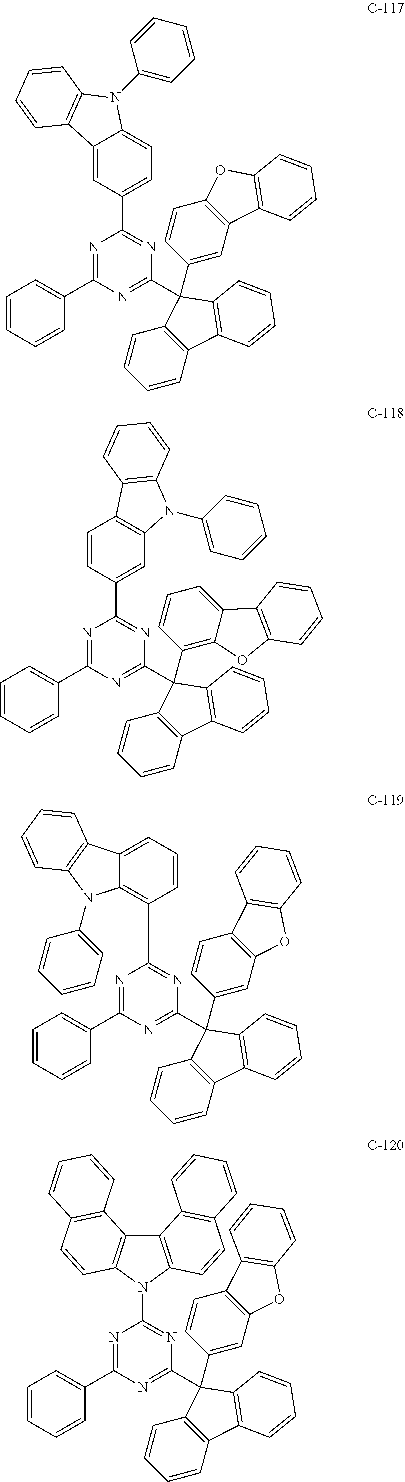

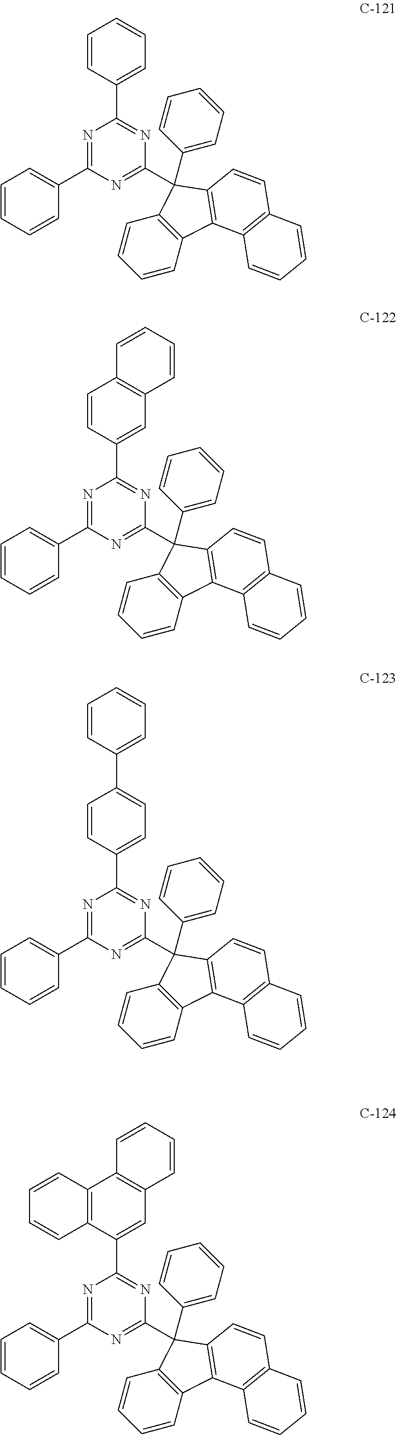 [SMILES: c1ccc(-c2nc(-c3ccccc3)nc(C3(c4ccccc4)c4ccccc4-c4c3ccc3ccccc43)n2)cc1 ; c1ccc(-c2nc(-c3ccc4ccccc4c3)nc(C3(c4ccccc4)c4ccccc4-c4c3ccc3ccccc43)n2)cc1 ; c1ccc(-c2ccc(-c3nc(-c4ccccc4)nc(C4(c5ccccc5)c5ccccc5-c5c4ccc4ccccc54)n3)cc2)cc1 ; c1ccc(-c2nc(-c3cc4ccccc4c4ccccc34)nc(C3(c4ccccc4)c4ccccc4-c4c3ccc3ccccc43)n2)cc1]

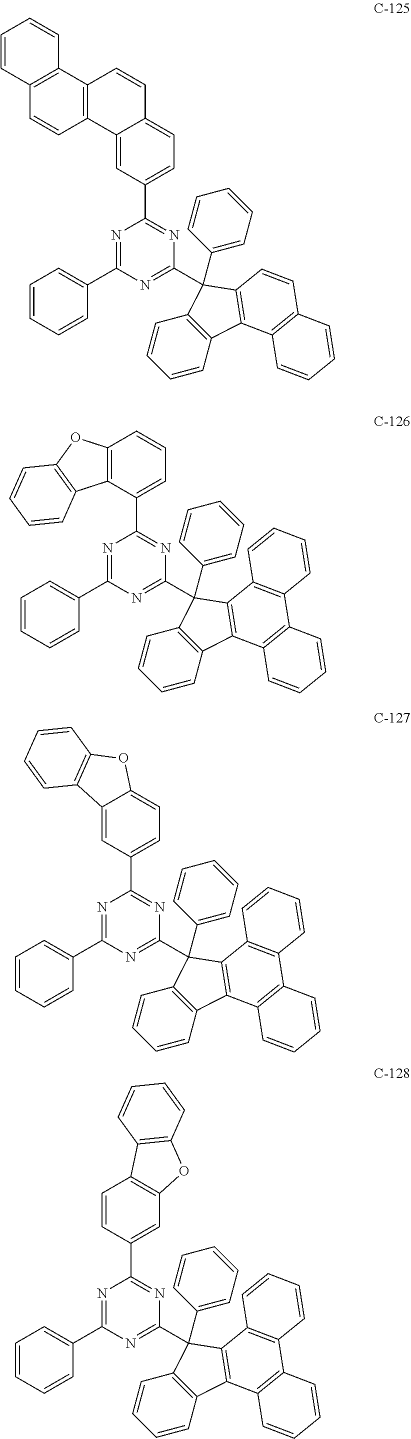

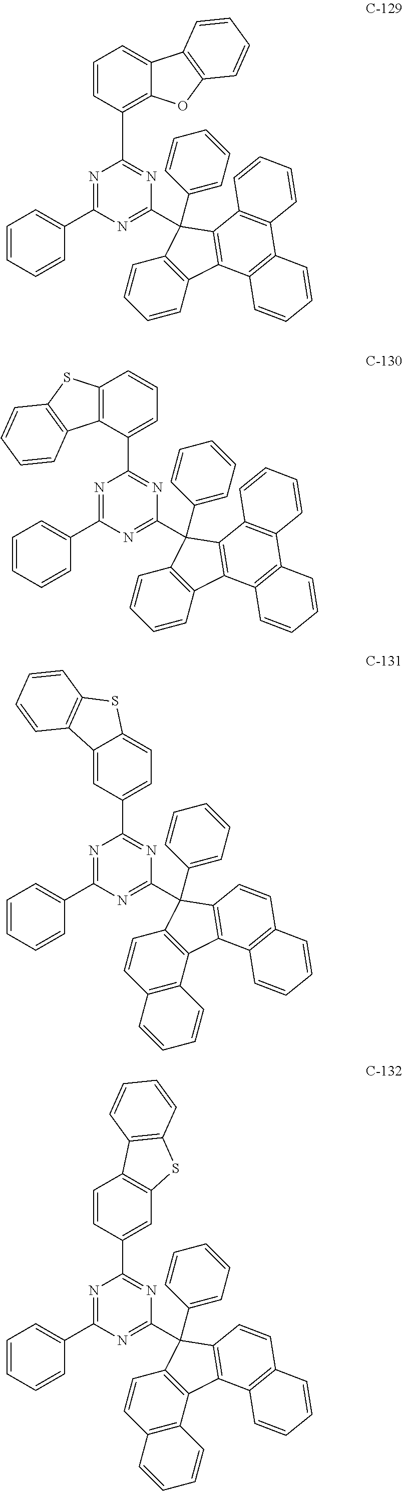

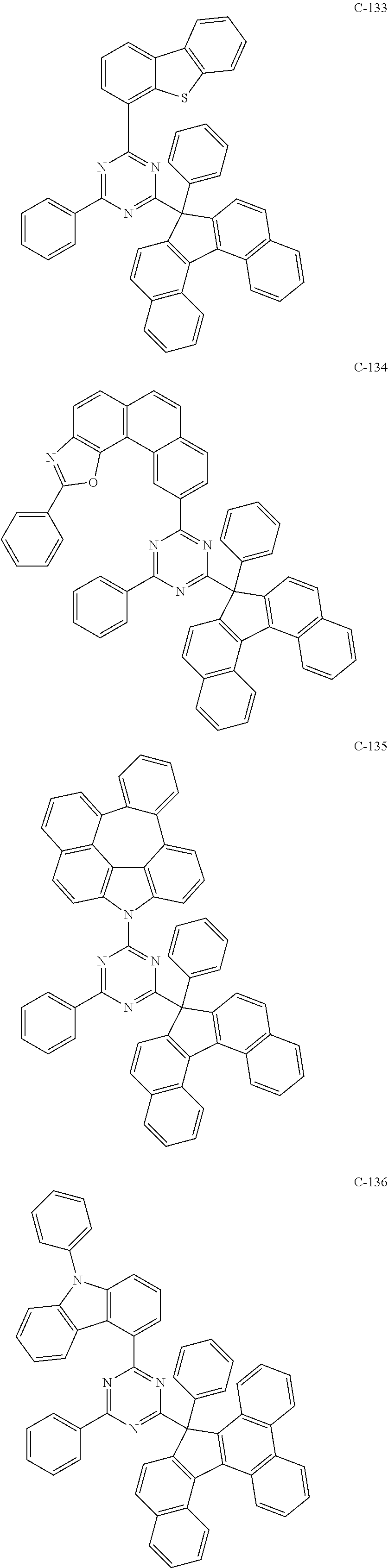

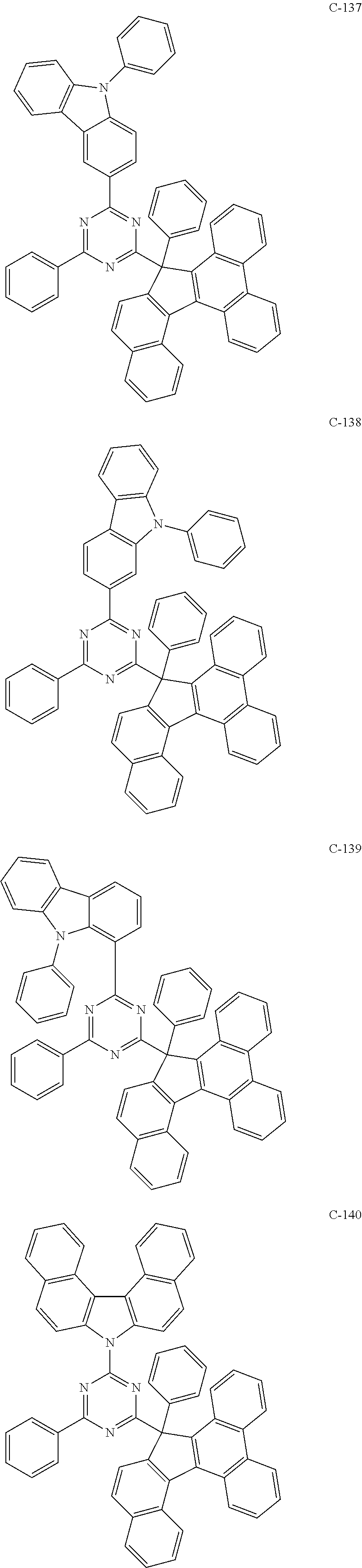

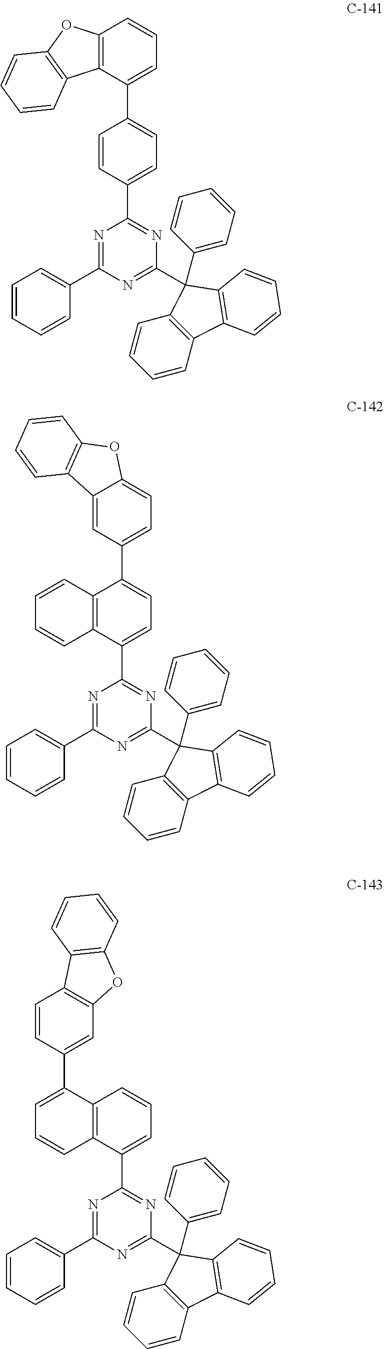 [SMILES: c1ccc(-c2nc(-c3ccc(-c4cccc5oc6ccccc6c45)cc3)nc(C3(c4ccccc4)c4ccccc4-c4ccccc43)n2)cc1 ; c1ccc(-c2nc(-c3ccc(-c4ccc5oc6ccccc6c5c4)c4ccccc34)nc(C3(c4ccccc4)c4ccccc4-c4ccccc43)n2)cc1 ; c1ccc(-c2nc(-c3cccc4c(-c5ccc6c(c5)oc5ccccc56)cccc34)nc(C3(c4ccccc4)c4ccccc4-c4ccccc43)n2)cc1]

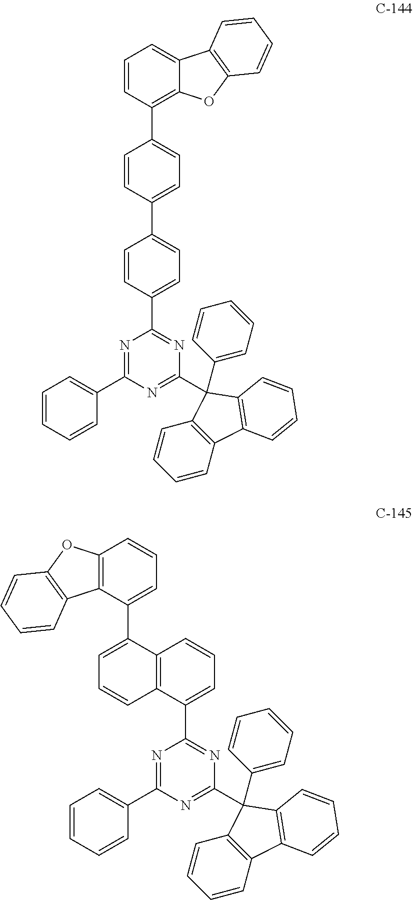 [SMILES: c1ccc(-c2nc(-c3ccc(-c4ccc(-c5cccc6c5oc5ccccc56)cc4)cc3)nc(C3(c4ccccc4)c4ccccc4-c4ccccc43)n2)cc1 ; c1ccc(-c2nc(-c3cccc4c(-c5cccc6oc7ccccc7c56)cccc34)nc(C3(c4ccccc4)c4ccccc4-c4ccccc43)n2)cc1]

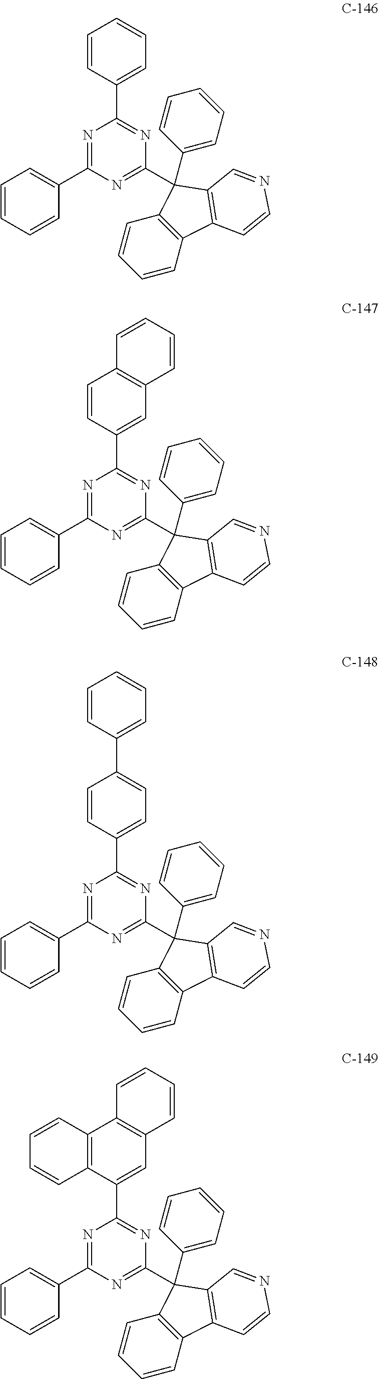

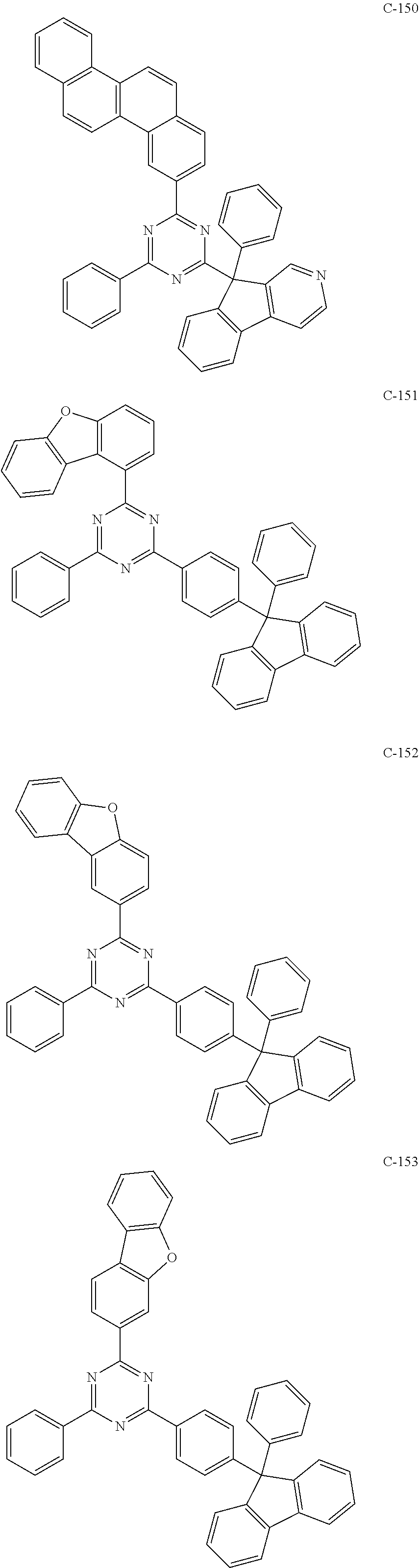 [SMILES: c1ccc(-c2nc(-c3ccc4ccc5c6ccccc6ccc5c4c3)nc(C3(c4ccccc4)c4ccccc4-c4ccncc43)n2)cc1 ; c1ccc(-c2nc(-c3ccc(C4(c5ccccc5)c5ccccc5-c5ccccc54)cc3)nc(-c3cccc4oc5ccccc5c34)n2)cc1 ; c1ccc(-c2nc(-c3ccc(C4(c5ccccc5)c5ccccc5-c5ccccc54)cc3)nc(-c3ccc4oc5ccccc5c4c3)n2)cc1 ; c1ccc(-c2nc(-c3ccc(C4(c5ccccc5)c5ccccc5-c5ccccc54)cc3)nc(-c3ccc4c(c3)oc3ccccc34)n2)cc1]

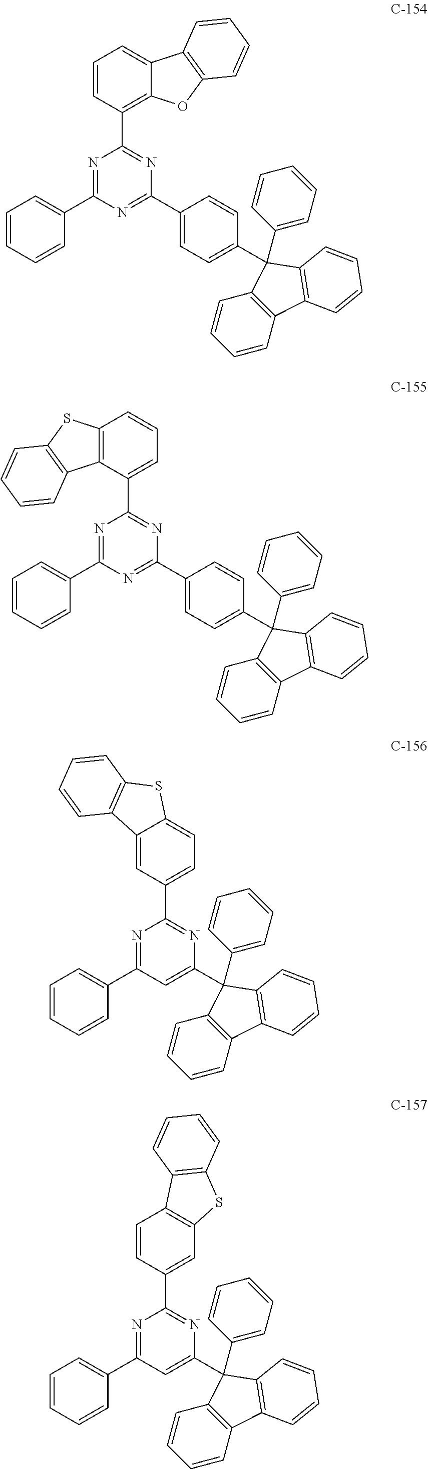

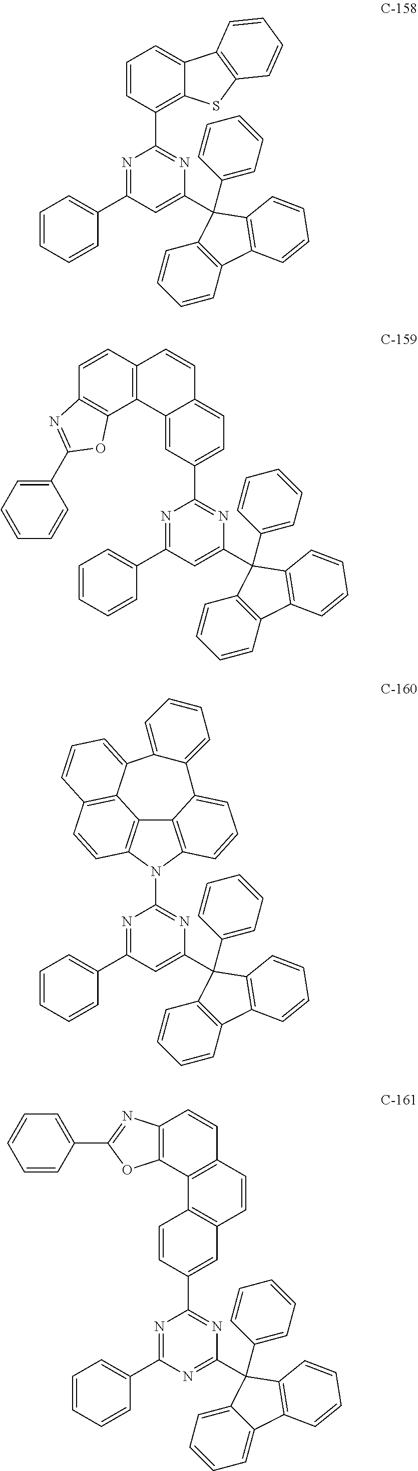

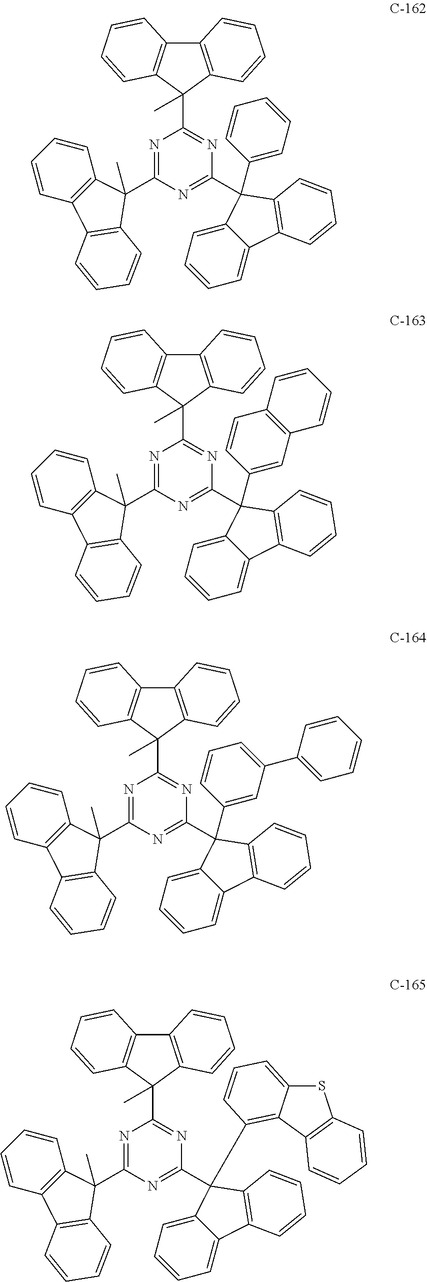

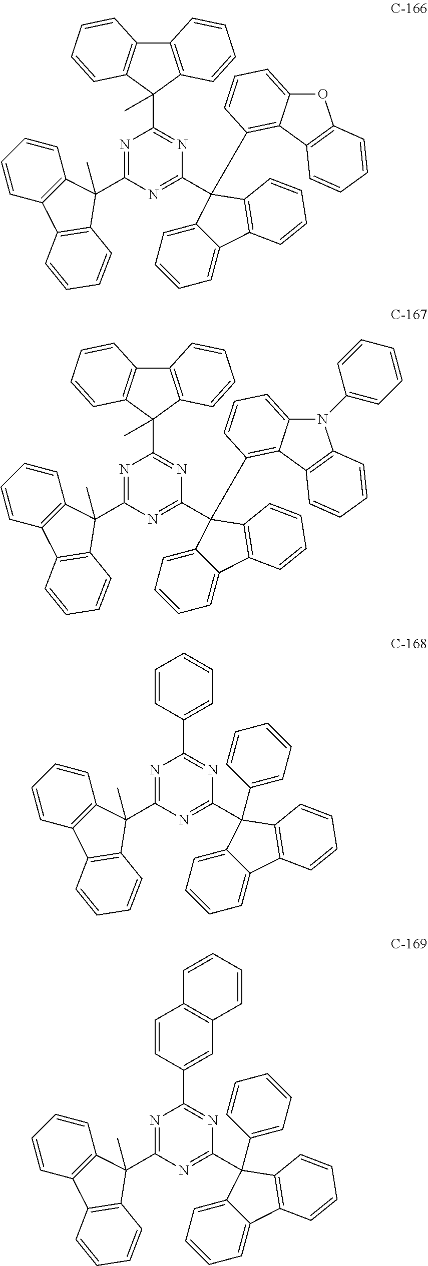

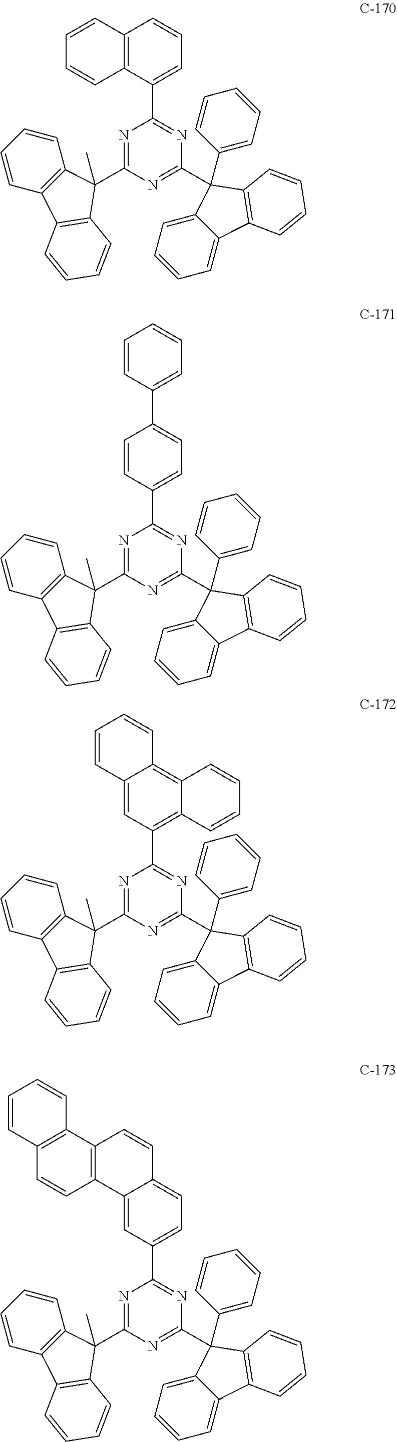

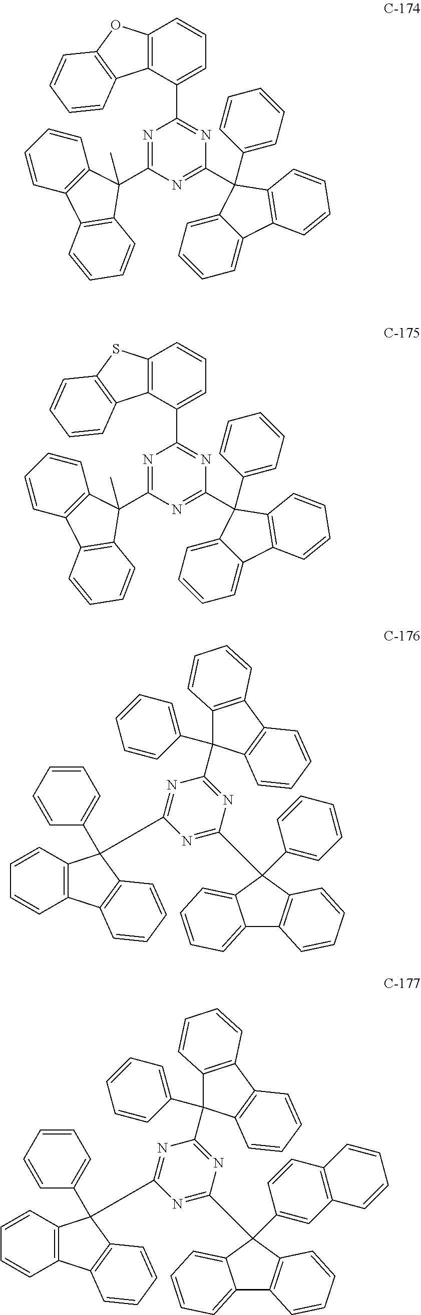 [SMILES: CC1(c2nc(-c3cccc4oc5ccccc5c34)nc(C3(c4ccccc4)c4ccccc4-c4ccccc43)n2)c2ccccc2-c2ccccc21 ; CC1(c2nc(-c3cccc4sc5ccccc5c34)nc(C3(c4ccccc4)c4ccccc4-c4ccccc43)n2)c2ccccc2-c2ccccc21 ; c1ccc(C2(c3nc(C4(c5ccccc5)c5ccccc5-c5ccccc54)nc(C4(c5ccccc5)c5ccccc5-c5ccccc54)n3)c3ccccc3-c3ccccc32)cc1 ; c1ccc(C2(c3nc(C4(c5ccccc5)c5ccccc5-c5ccccc54)nc(C4(c5ccc6ccccc6c5)c5ccccc5-c5ccccc54)n3)c3ccccc3-c3ccccc32)cc1]

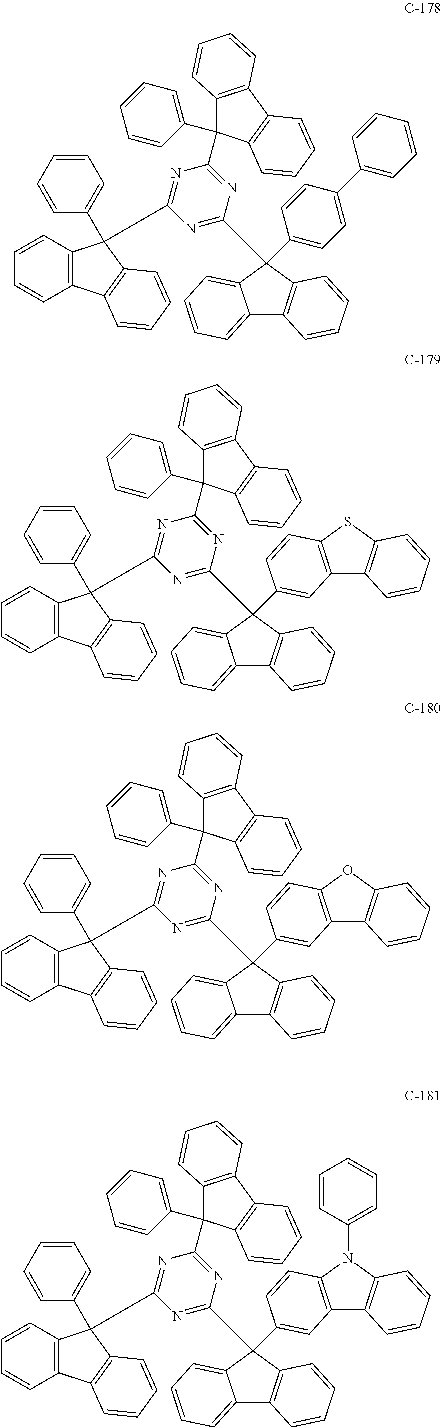

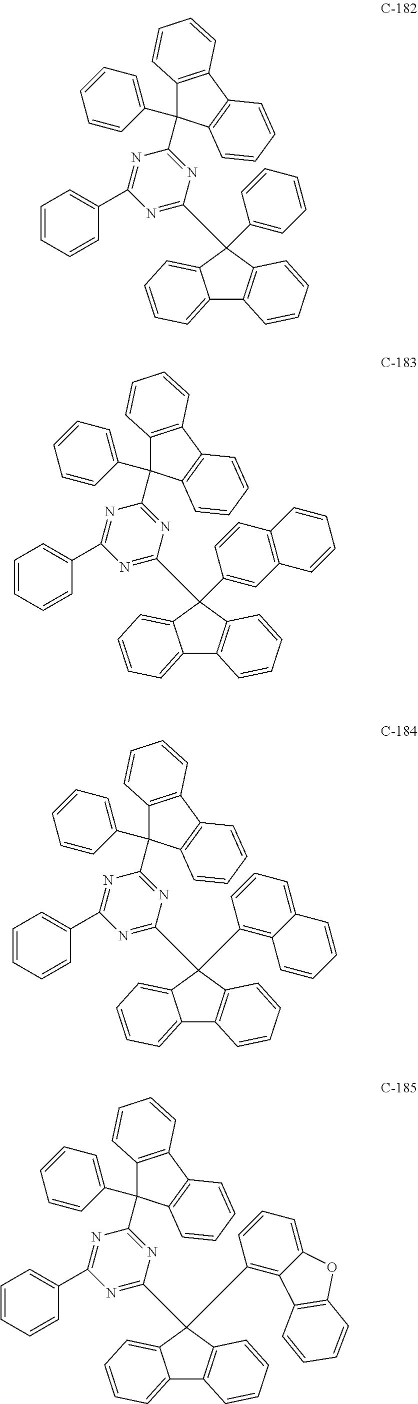 [SMILES: c1ccc(-c2nc(C3(c4ccccc4)c4ccccc4-c4ccccc43)nc(C3(c4ccccc4)c4ccccc4-c4ccccc43)n2)cc1 ; c1ccc(-c2nc(C3(c4ccccc4)c4ccccc4-c4ccccc43)nc(C3(c4ccc5ccccc5c4)c4ccccc4-c4ccccc43)n2)cc1 ; c1ccc(-c2nc(C3(c4ccccc4)c4ccccc4-c4ccccc43)nc(C3(c4cccc5ccccc45)c4ccccc4-c4ccccc43)n2)cc1 ; c1ccc(-c2nc(C3(c4ccccc4)c4ccccc4-c4ccccc43)nc(C3(c4cccc5oc6ccccc6c45)c4ccccc4-c4ccccc43)n2)cc1]

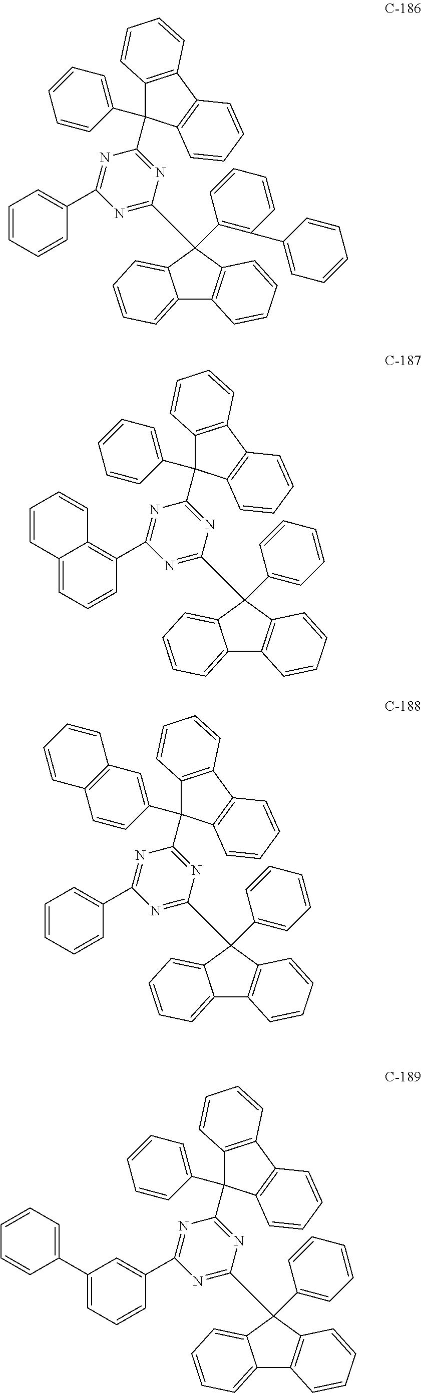

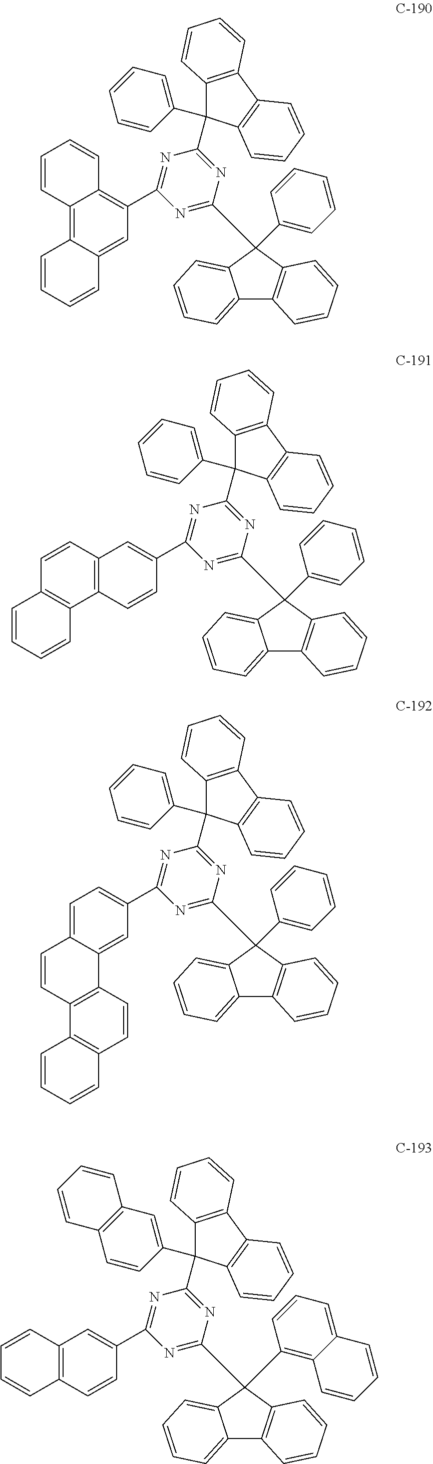 [SMILES: c1ccc(C2(c3nc(-c4cc5ccccc5c5ccccc45)nc(C4(c5ccccc5)c5ccccc5-c5ccccc54)n3)c3ccccc3-c3ccccc32)cc1 ; c1ccc(C2(c3nc(-c4ccc5c(ccc6ccccc65)c4)nc(C4(c5ccccc5)c5ccccc5-c5ccccc54)n3)c3ccccc3-c3ccccc32)cc1 ; c1ccc(C2(c3nc(-c4ccc5ccc6c7ccccc7ccc6c5c4)nc(C4(c5ccccc5)c5ccccc5-c5ccccc54)n3)c3ccccc3-c3ccccc32)cc1 ; c1ccc2c(c1)-c1ccccc1C2(c1ccc2ccccc2c1)c1nc(-c2ccc3ccccc3c2)nc(C2(c3cccc4ccccc34)c3ccccc3-c3ccccc32)n1]

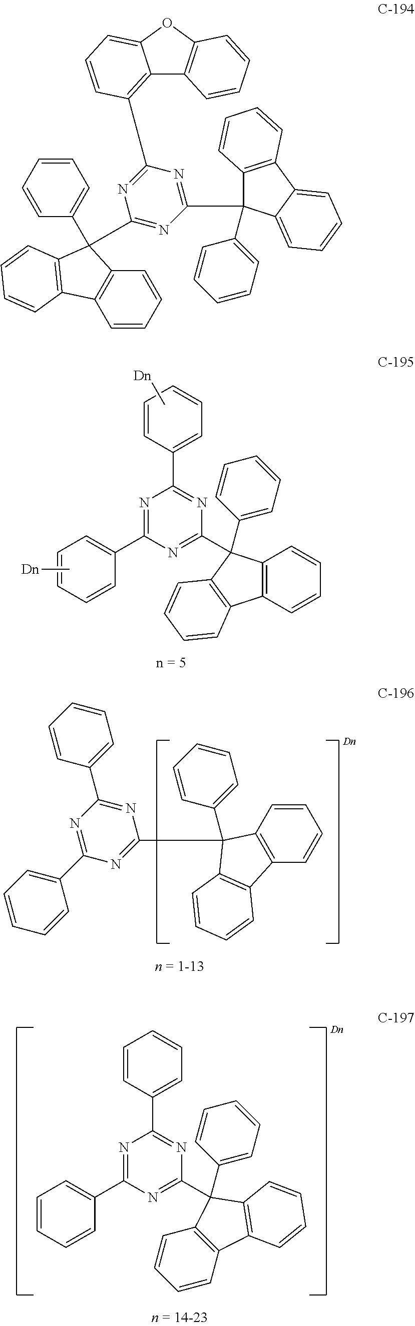

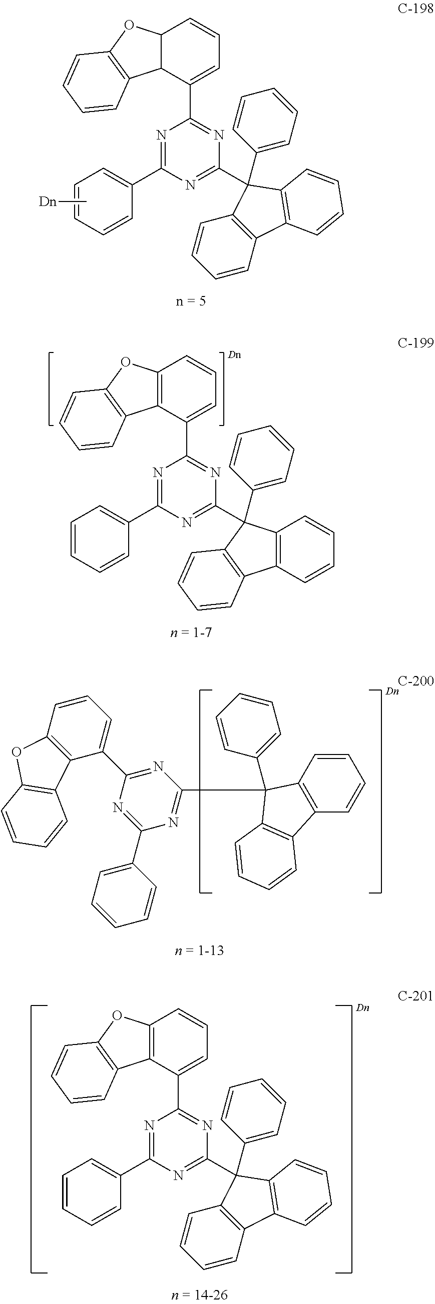

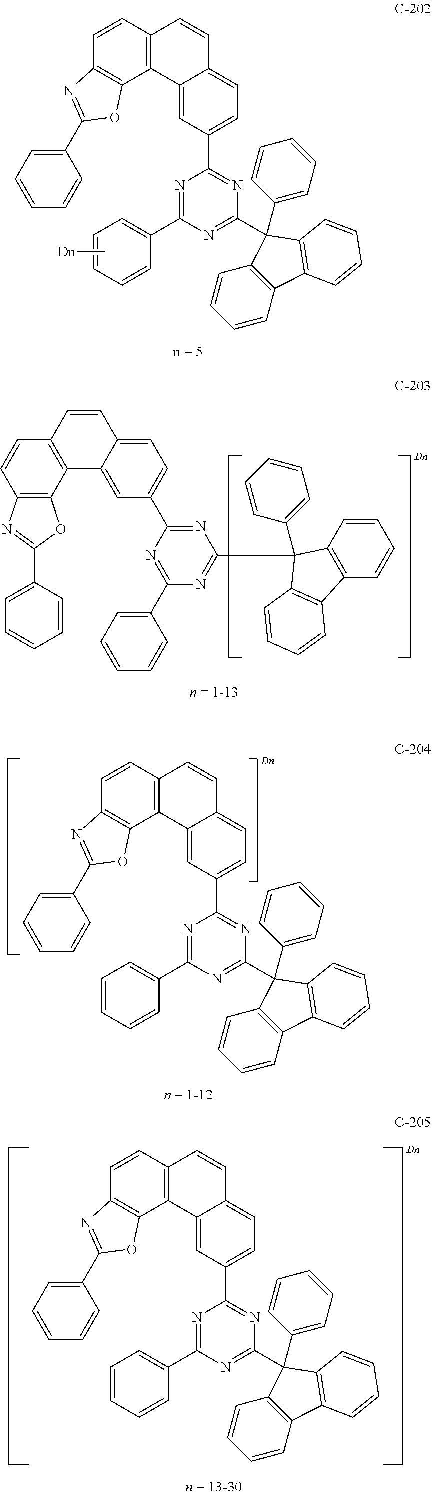

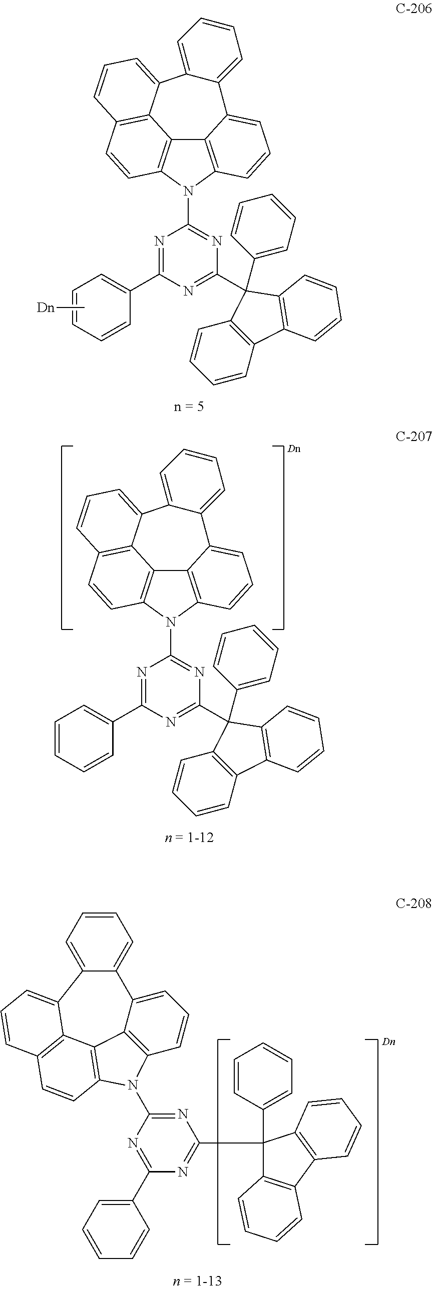

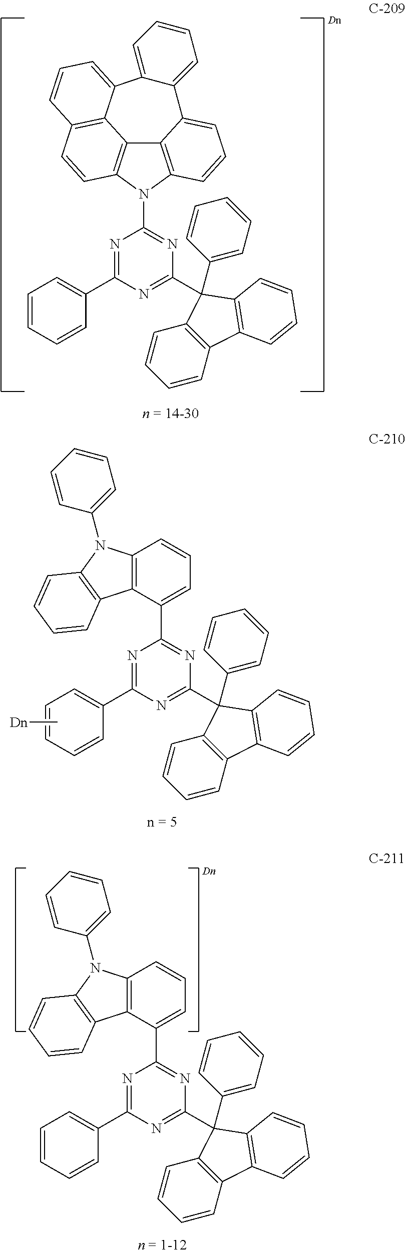

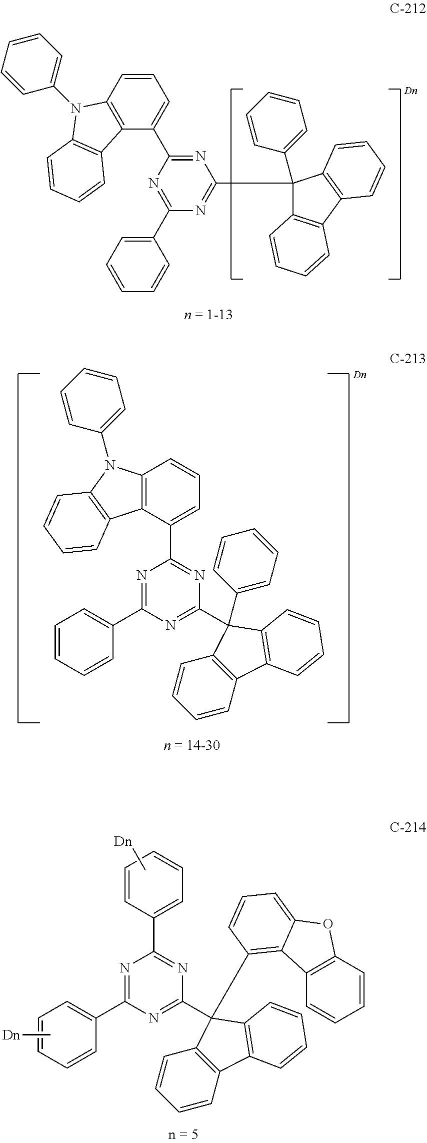

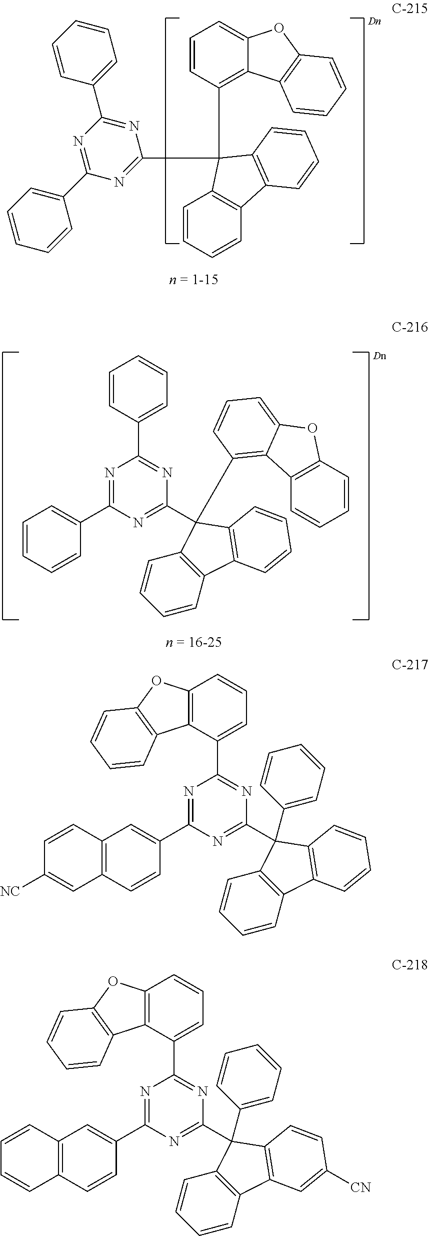

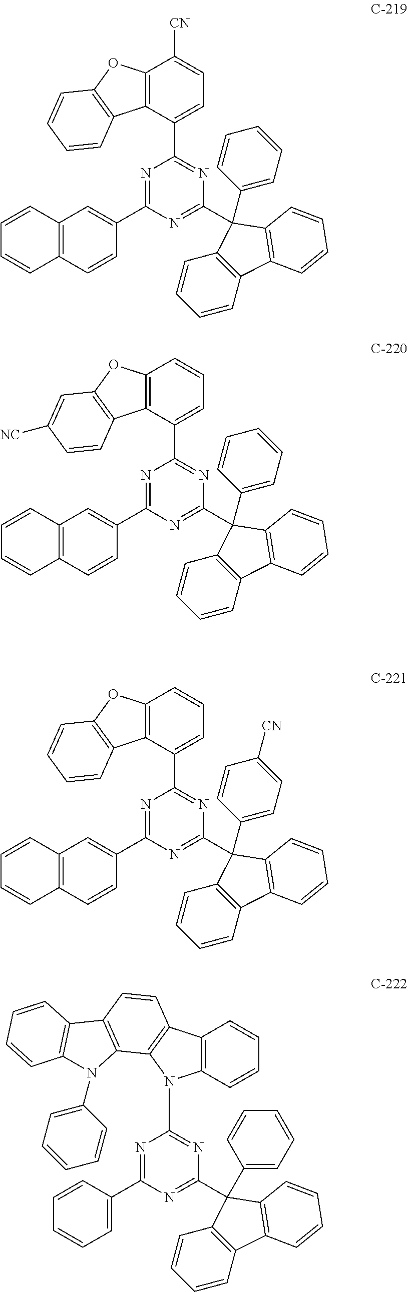 [SMILES: N#Cc1ccc(-c2nc(-c3ccc4ccccc4c3)nc(C3(c4ccccc4)c4ccccc4-c4ccccc43)n2)c2c1oc1ccccc12 ; N#Cc1ccc2c(c1)oc1cccc(-c3nc(-c4ccc5ccccc5c4)nc(C4(c5ccccc5)c5ccccc5-c5ccccc54)n3)c12 ; N#Cc1ccc(C2(c3nc(-c4ccc5ccccc5c4)nc(-c4cccc5oc6ccccc6c45)n3)c3ccccc3-c3ccccc32)cc1 ; c1ccc(-c2nc(-n3c4ccccc4c4ccc5c6ccccc6n(-c6ccccc6)c5c43)nc(C3(c4ccccc4)c4ccccc4-c4ccccc43)n2)cc1]

In the formulas above. D represents deuterium, and n represents the number of deuterium.

The compound represented by formula 2 may be at least one selected from the following compounds, but is not limited thereto.

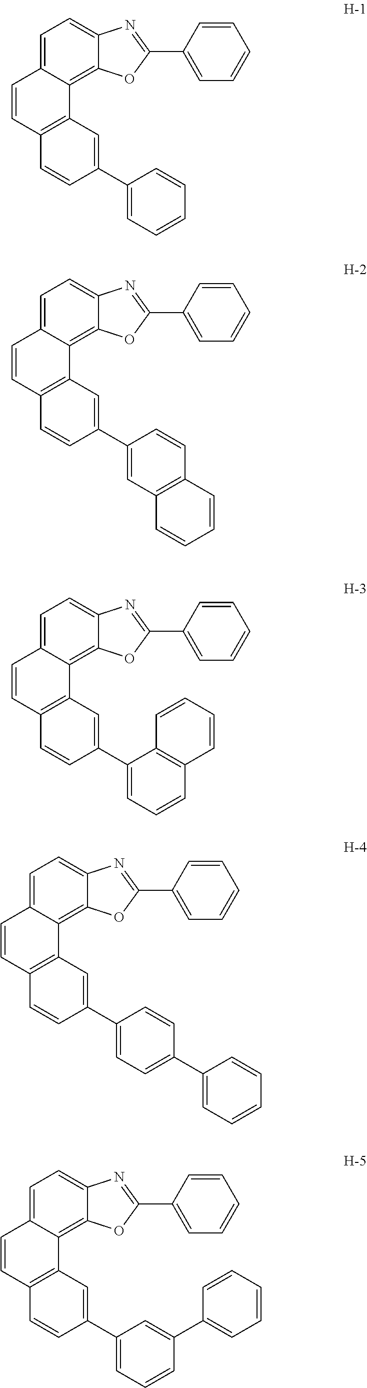 [SMILES: c1ccc(-c2ccc3ccc4ccc5nc(-c6ccccc6)oc5c4c3c2)cc1 ; c1ccc(-c2nc3ccc4ccc5ccc(-c6ccc7ccccc7c6)cc5c4c3o2)cc1 ; c1ccc(-c2nc3ccc4ccc5ccc(-c6cccc7ccccc67)cc5c4c3o2)cc1 ; c1ccc(-c2ccc(-c3ccc4ccc5ccc6nc(-c7ccccc7)oc6c5c4c3)cc2)cc1 ; c1ccc(-c2cccc(-c3ccc4ccc5ccc6nc(-c7ccccc7)oc6c5c4c3)c2)cc1]

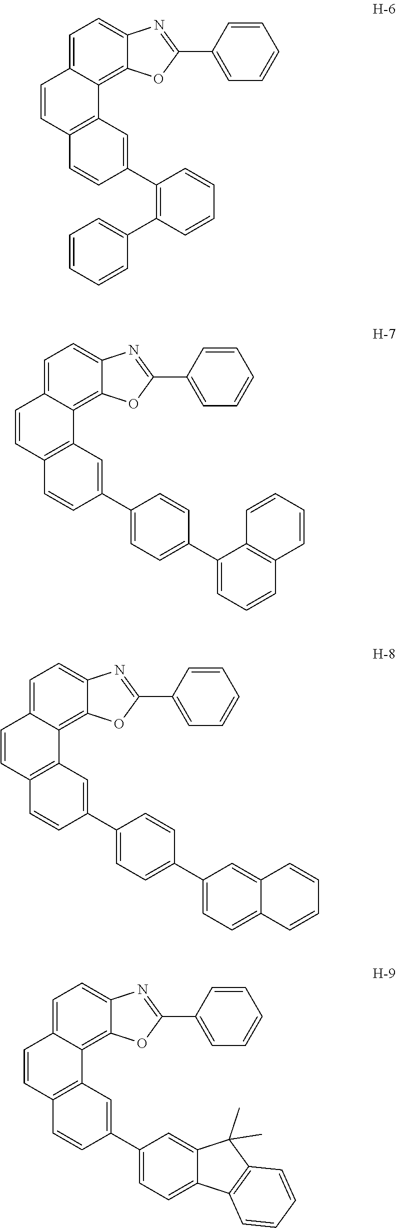 [SMILES: c1ccc(-c2nc3ccc4ccc5ccc(-c6ccccc6-c6ccccc6)cc5c4c3o2)cc1 ; c1ccc(-c2nc3ccc4ccc5ccc(-c6ccc(-c7cccc8ccccc78)cc6)cc5c4c3o2)cc1 ; c1ccc(-c2nc3ccc4ccc5ccc(-c6ccc(-c7ccc8ccccc8c7)cc6)cc5c4c3o2)cc1 ; CC1(C)c2ccccc2-c2ccc(-c3ccc4ccc5ccc6nc(-c7ccccc7)oc6c5c4c3)cc21]

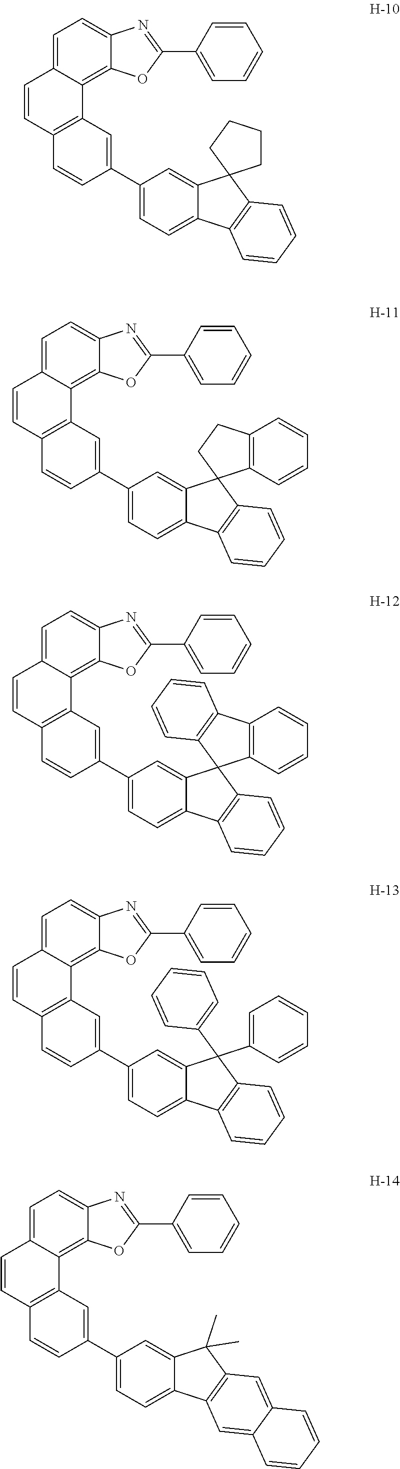 [SMILES: c1ccc(-c2nc3ccc4ccc5ccc(-c6ccc7c(c6)C6(CCCC6)c6ccccc6-7)cc5c4c3o2)cc1 ; c1ccc(-c2nc3ccc4ccc5ccc(-c6ccc7c(c6)C6(CCc8ccccc86)c6ccccc6-7)cc5c4c3o2)cc1 ; c1ccc(-c2nc3ccc4ccc5ccc(-c6ccc7c(c6)C6(c8ccccc8-c8ccccc86)c6ccccc6-7)cc5c4c3o2)cc1 ; c1ccc(-c2nc3ccc4ccc5ccc(-c6ccc7c(c6)C(c6ccccc6)(c6ccccc6)c6ccccc6-7)cc5c4c3o2)cc1 ; CC1(C)c2cc(-c3ccc4ccc5ccc6nc(-c7ccccc7)oc6c5c4c3)ccc2-c2cc3ccccc3cc21]

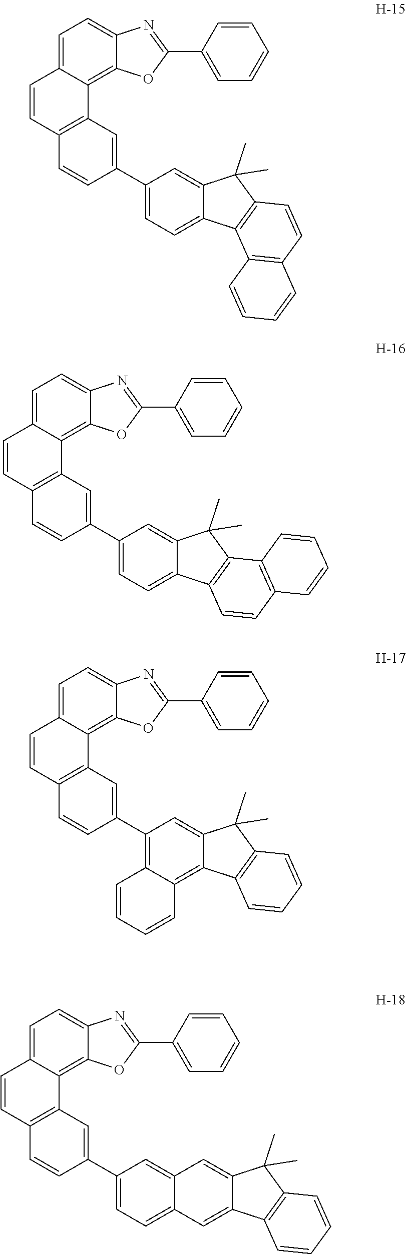 [SMILES: CC1(C)c2cc(-c3ccc4ccc5ccc6nc(-c7ccccc7)oc6c5c4c3)ccc2-c2c1ccc1ccccc21 ; CC1(C)c2cc(-c3ccc4ccc5ccc6nc(-c7ccccc7)oc6c5c4c3)ccc2-c2ccc3ccccc3c21 ; CC1(C)c2ccccc2-c2c1cc(-c1ccc3ccc4ccc5nc(-c6ccccc6)oc5c4c3c1)c1ccccc21 ; CC1(C)c2ccccc2-c2cc3ccc(-c4ccc5ccc6ccc7nc(-c8ccccc8)oc7c6c5c4)cc3cc21]

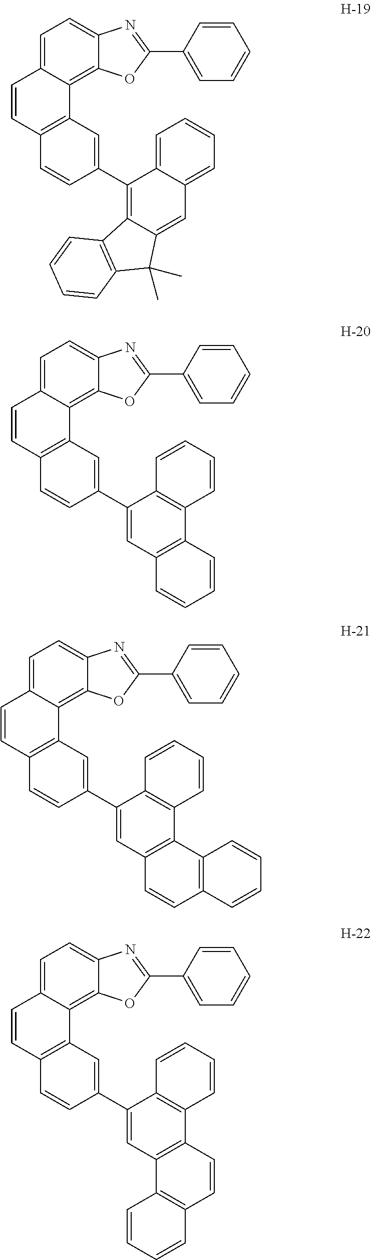

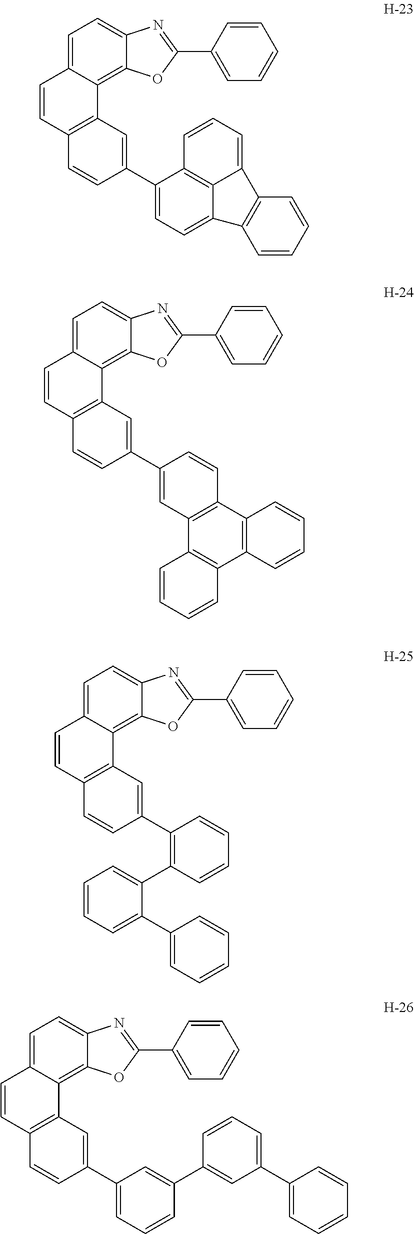

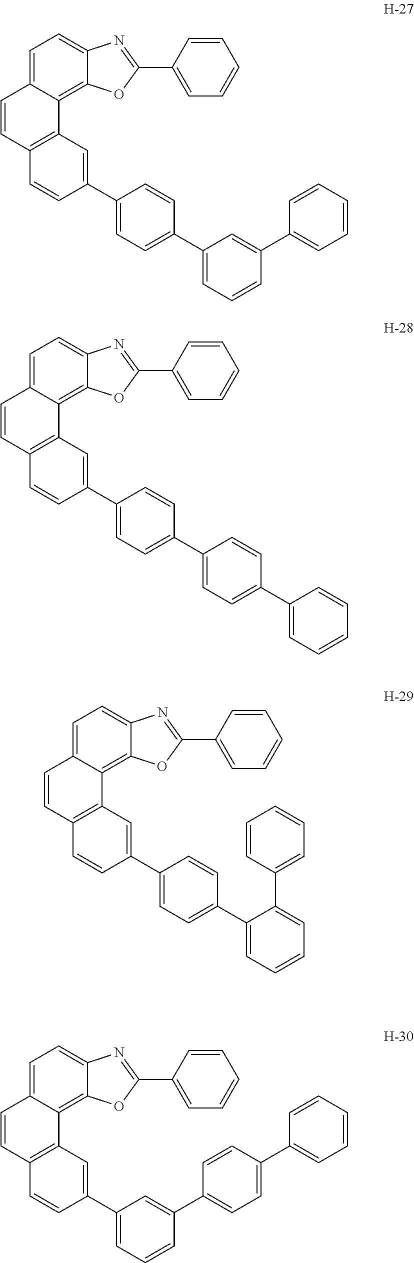

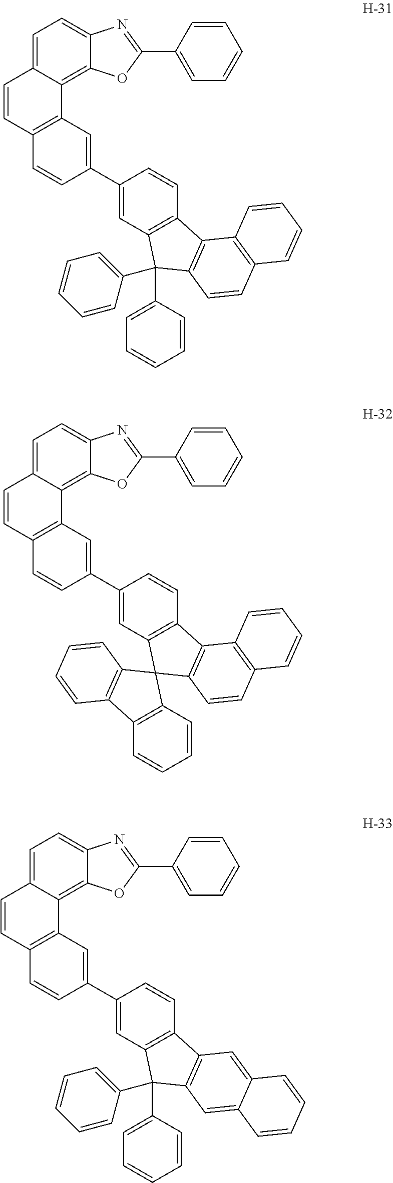 [SMILES: c1ccc(-c2nc3ccc4ccc5ccc(-c6ccc7c(c6)C(c6ccccc6)(c6ccccc6)c6ccc8ccccc8c6-7)cc5c4c3o2)cc1 ; c1ccc(-c2nc3ccc4ccc5ccc(-c6ccc7c(c6)C6(c8ccccc8-c8ccccc86)c6ccc8ccccc8c6-7)cc5c4c3o2)cc1 ; c1ccc(-c2nc3ccc4ccc5ccc(-c6ccc7c(c6)C(c6ccccc6)(c6ccccc6)c6cc8ccccc8cc6-7)cc5c4c3o2)cc1]

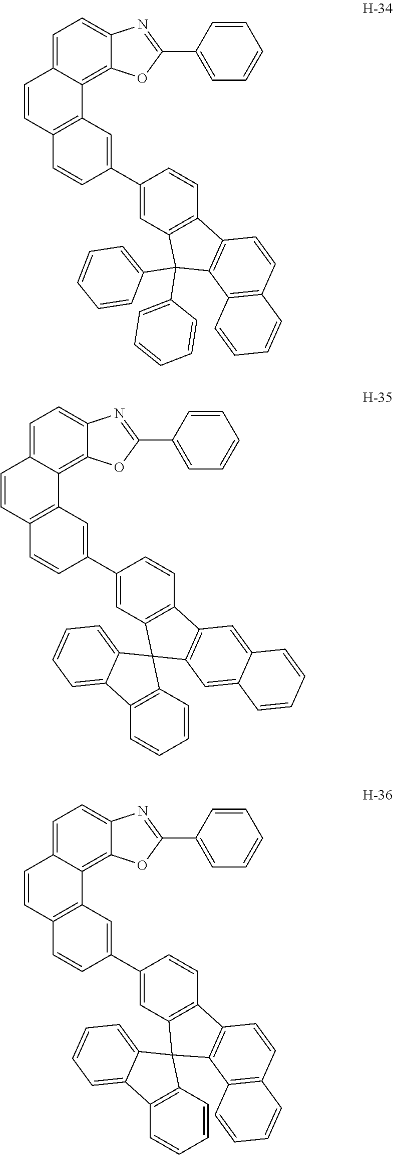 [SMILES: c1ccc(-c2nc3ccc4ccc5ccc(-c6ccc7c(c6)C(c6ccccc6)(c6ccccc6)c6c-7ccc7ccccc67)cc5c4c3o2)cc1 ; c1ccc(-c2nc3ccc4ccc5ccc(-c6ccc7c(c6)C6(c8ccccc8-c8ccccc86)c6cc8ccccc8cc6-7)cc5c4c3o2)cc1 ; c1ccc(-c2nc3ccc4ccc5ccc(-c6ccc7c(c6)C6(c8ccccc8-c8ccccc86)c6c-7ccc7ccccc67)cc5c4c3o2)cc1]

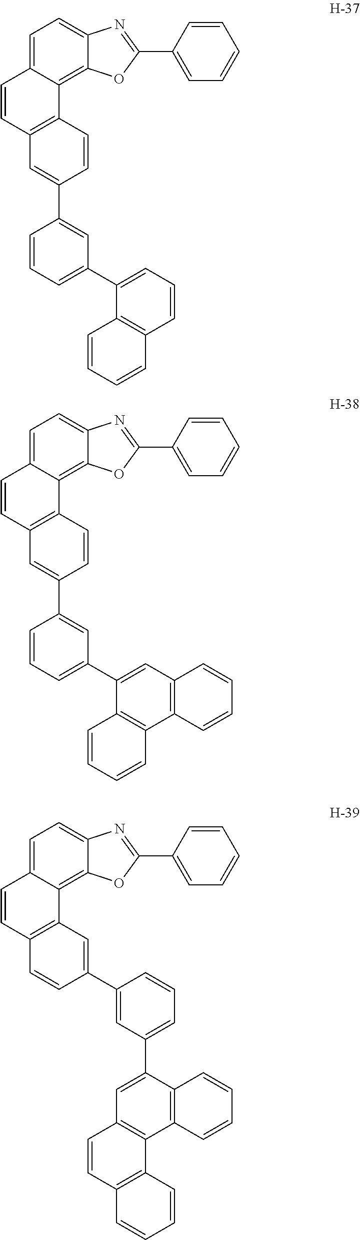

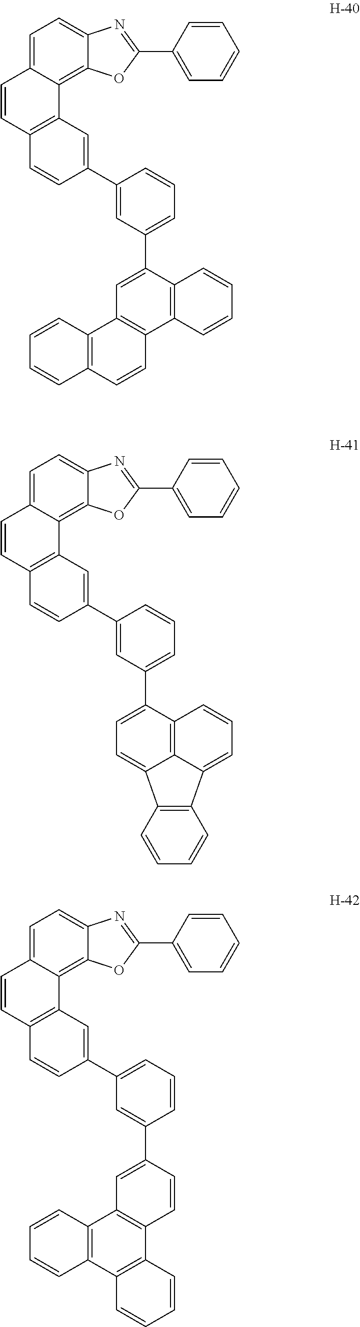 [SMILES: c1ccc(-c2nc3ccc4ccc5ccc(-c6cccc(-c7cc8c9ccccc9ccc8c8ccccc78)c6)cc5c4c3o2)cc1 ; c1ccc(-c2nc3ccc4ccc5ccc(-c6cccc(-c7ccc8c9c(cccc79)-c7ccccc7-8)c6)cc5c4c3o2)cc1 ; c1ccc(-c2nc3ccc4ccc5ccc(-c6cccc(-c7ccc8c9ccccc9c9ccccc9c8c7)c6)cc5c4c3o2)cc1]

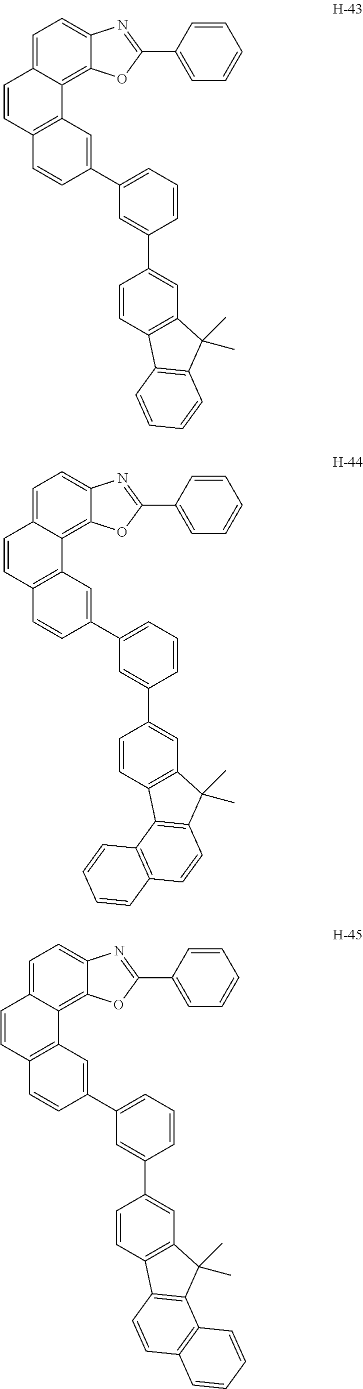

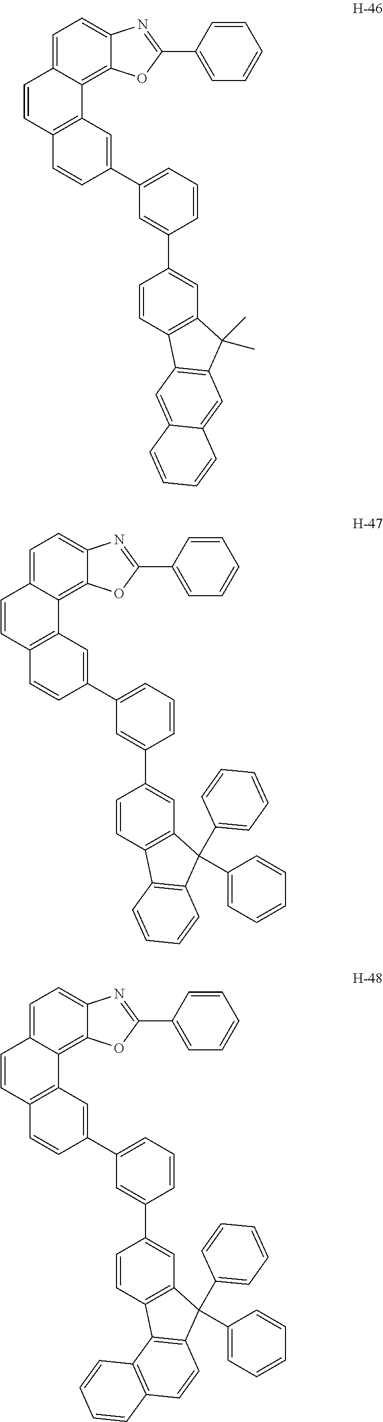 [SMILES: CC1(C)c2cc(-c3cccc(-c4ccc5ccc6ccc7nc(-c8ccccc8)oc7c6c5c4)c3)ccc2-c2cc3ccccc3cc21 ; c1ccc(-c2nc3ccc4ccc5ccc(-c6cccc(-c7ccc8c(c7)C(c7ccccc7)(c7ccccc7)c7ccccc7-8)c6)cc5c4c3o2)cc1 ; c1ccc(-c2nc3ccc4ccc5ccc(-c6cccc(-c7ccc8c(c7)C(c7ccccc7)(c7ccccc7)c7ccc9ccccc9c7-8)c6)cc5c4c3o2)cc1]

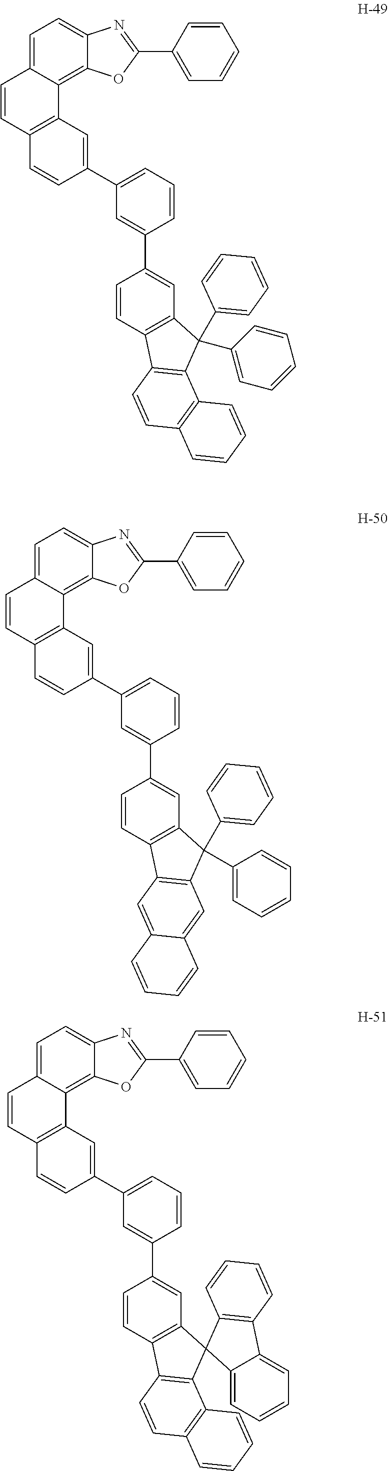

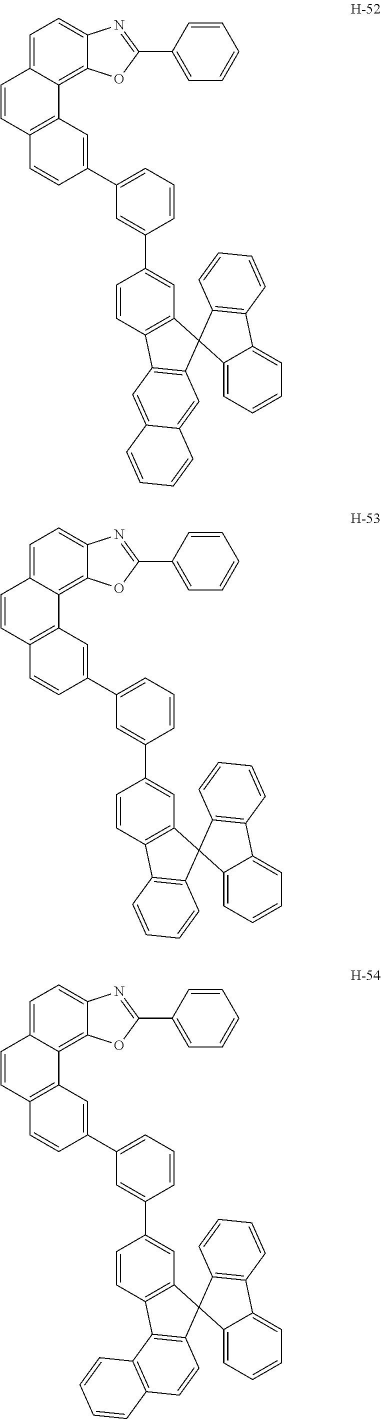 [SMILES: c1ccc(-c2nc3ccc4ccc5ccc(-c6cccc(-c7ccc8c(c7)C7(c9ccccc9-c9ccccc97)c7cc9ccccc9cc7-8)c6)cc5c4c3o2)cc1 ; c1ccc(-c2nc3ccc4ccc5ccc(-c6cccc(-c7ccc8c(c7)C7(c9ccccc9-c9ccccc97)c7ccccc7-8)c6)cc5c4c3o2)cc1 ; c1ccc(-c2nc3ccc4ccc5ccc(-c6cccc(-c7ccc8c(c7)C7(c9ccccc9-c9ccccc97)c7ccc9ccccc9c7-8)c6)cc5c4c3o2)cc1]

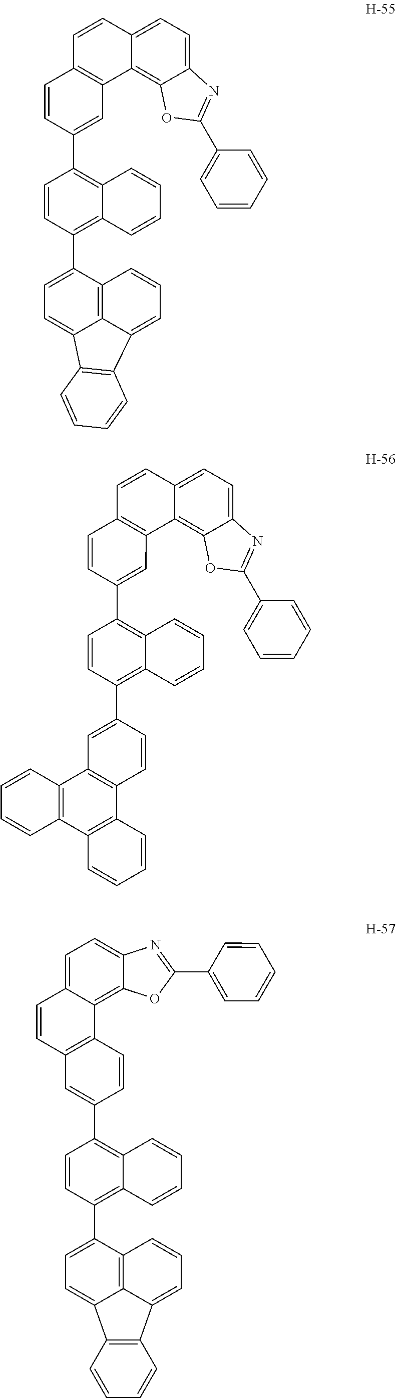 [SMILES: c1ccc(-c2nc3ccc4ccc5ccc(-c6ccc(-c7ccc8c9c(cccc79)-c7ccccc7-8)c7ccccc67)cc5c4c3o2)cc1 ; c1ccc(-c2nc3ccc4ccc5ccc(-c6ccc(-c7ccc8c9ccccc9c9ccccc9c8c7)c7ccccc67)cc5c4c3o2)cc1 ; c1ccc(-c2nc3ccc4ccc5cc(-c6ccc(-c7ccc8c9c(cccc79)-c7ccccc7-8)c7ccccc67)ccc5c4c3o2)cc1]

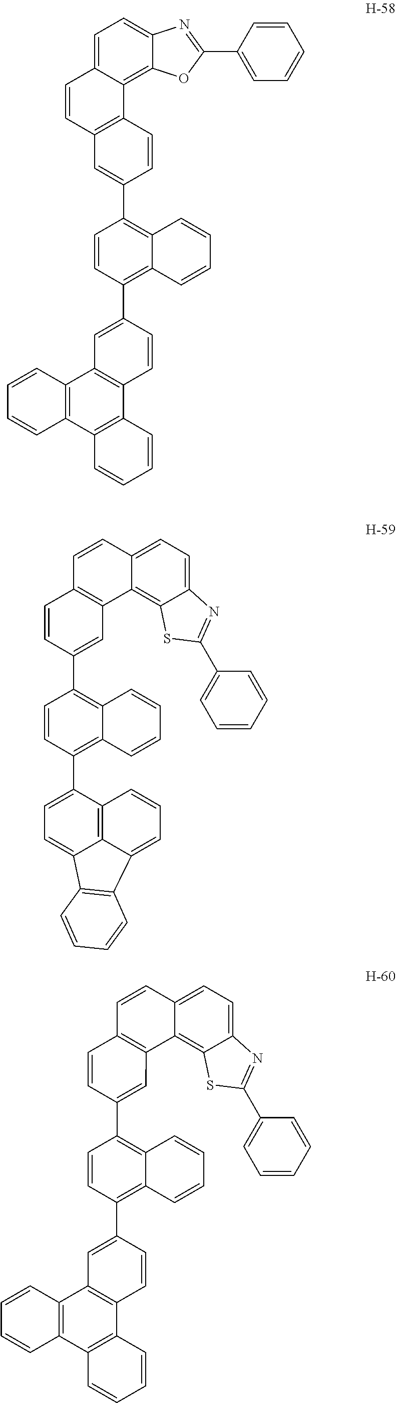 [SMILES: c1ccc(-c2nc3ccc4ccc5cc(-c6ccc(-c7ccc8c9ccccc9c9ccccc9c8c7)c7ccccc67)ccc5c4c3o2)cc1 ; c1ccc(-c2nc3ccc4ccc5ccc(-c6ccc(-c7ccc8c9c(cccc79)-c7ccccc7-8)c7ccccc67)cc5c4c3s2)cc1 ; c1ccc(-c2nc3ccc4ccc5ccc(-c6ccc(-c7ccc8c9ccccc9c9ccccc9c8c7)c7ccccc67)cc5c4c3s2)cc1]

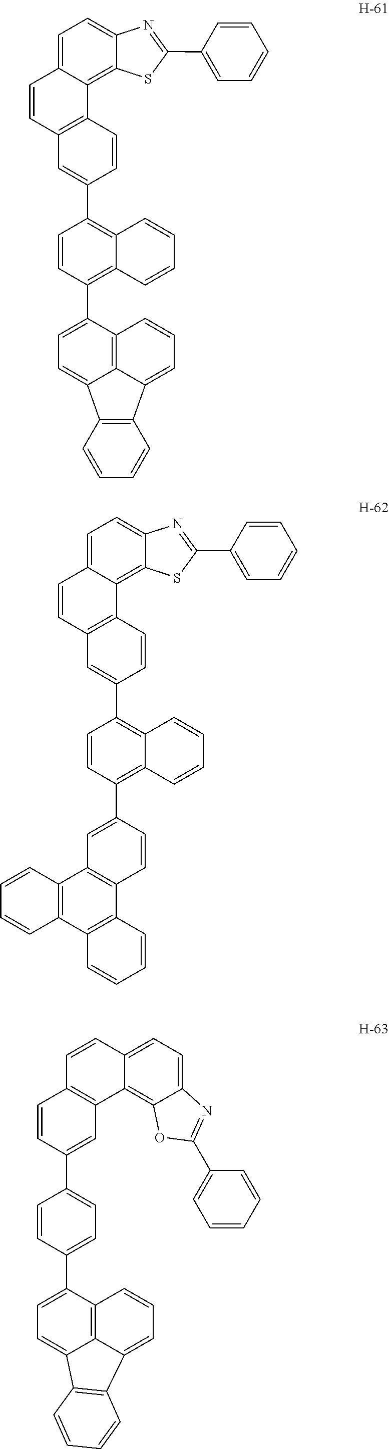

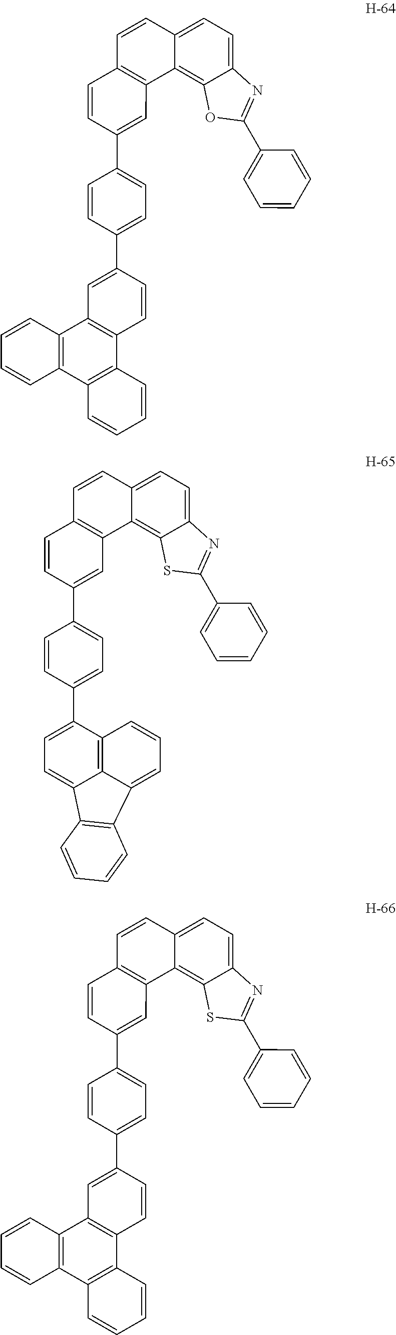

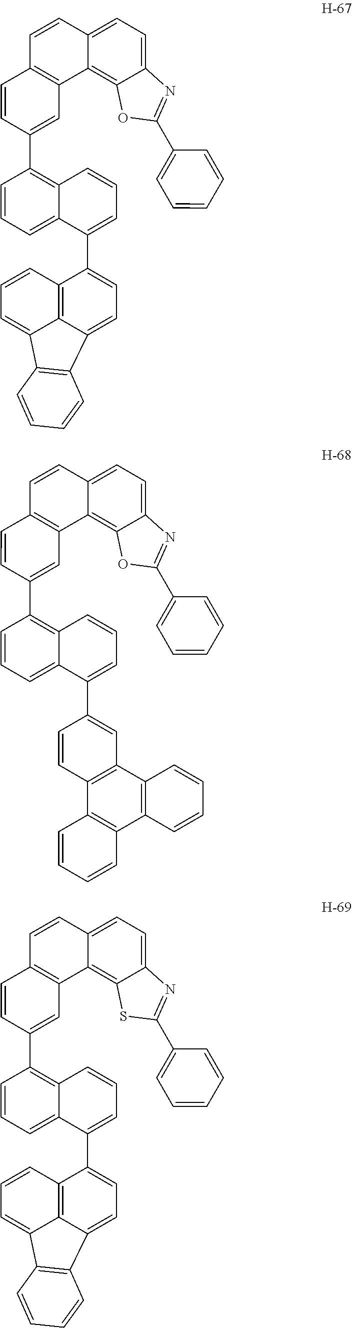

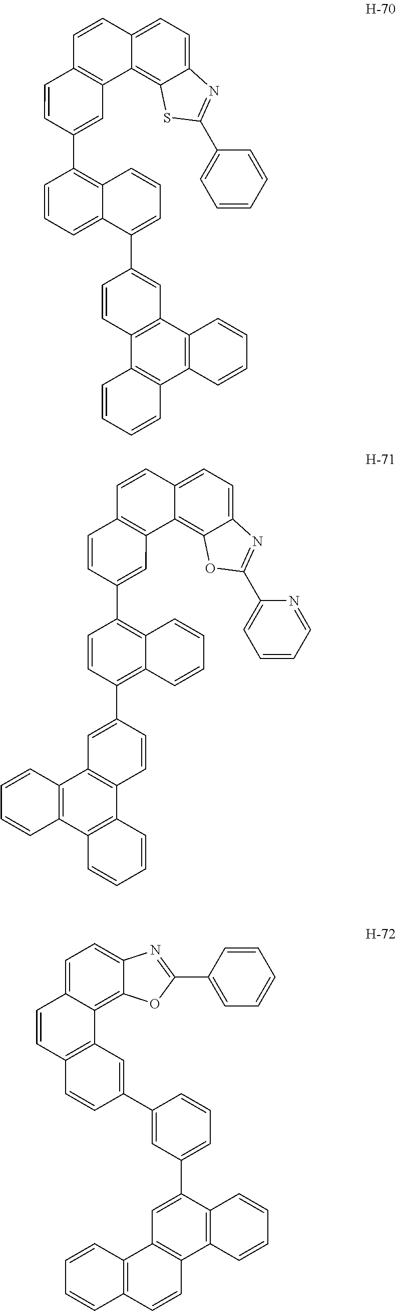

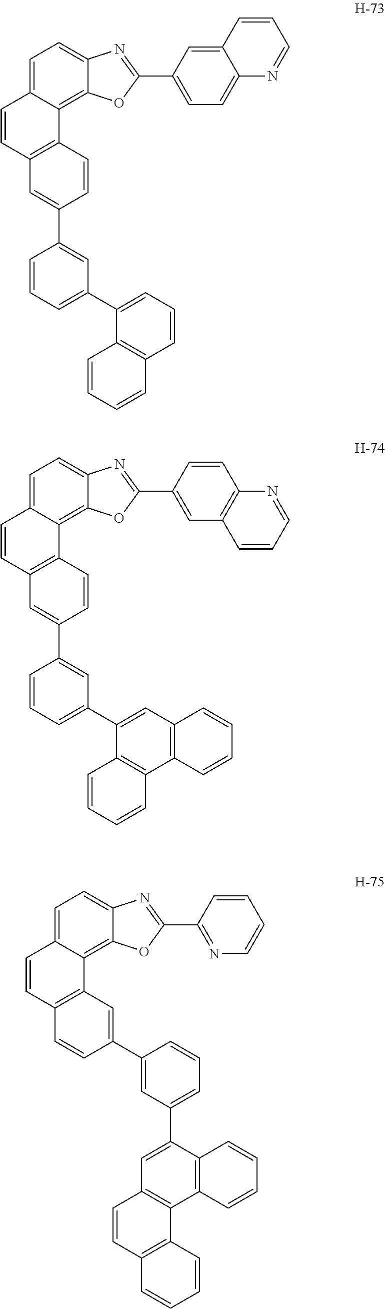 [SMILES: c1cc(-c2ccc3c(ccc4ccc5nc(-c6ccc7ncccc7c6)oc5c43)c2)cc(-c2cccc3ccccc23)c1 ; c1cc(-c2ccc3c(ccc4ccc5nc(-c6ccc7ncccc7c6)oc5c43)c2)cc(-c2cc3ccccc3c3ccccc23)c1 ; c1ccc(-c2nc3ccc4ccc5ccc(-c6cccc(-c7cc8ccc9ccccc9c8c8ccccc78)c6)cc5c4c3o2)nc1]

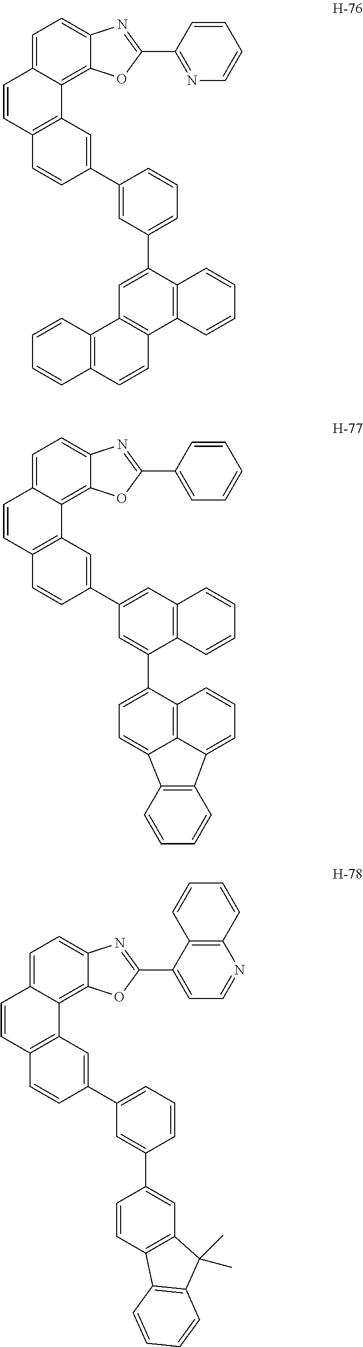

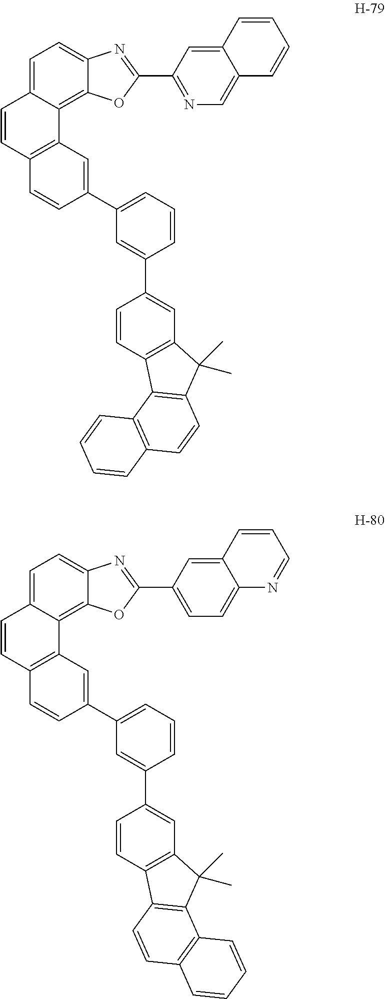 [SMILES: CC1(C)c2cc(-c3cccc(-c4ccc5ccc6ccc7nc(-c8cc9ccccc9cn8)oc7c6c5c4)c3)ccc2-c2c1ccc1ccccc21 ; CC1(C)c2cc(-c3cccc(-c4ccc5ccc6ccc7nc(-c8ccc9ncccc9c8)oc7c6c5c4)c3)ccc2-c2ccc3ccccc3c21]

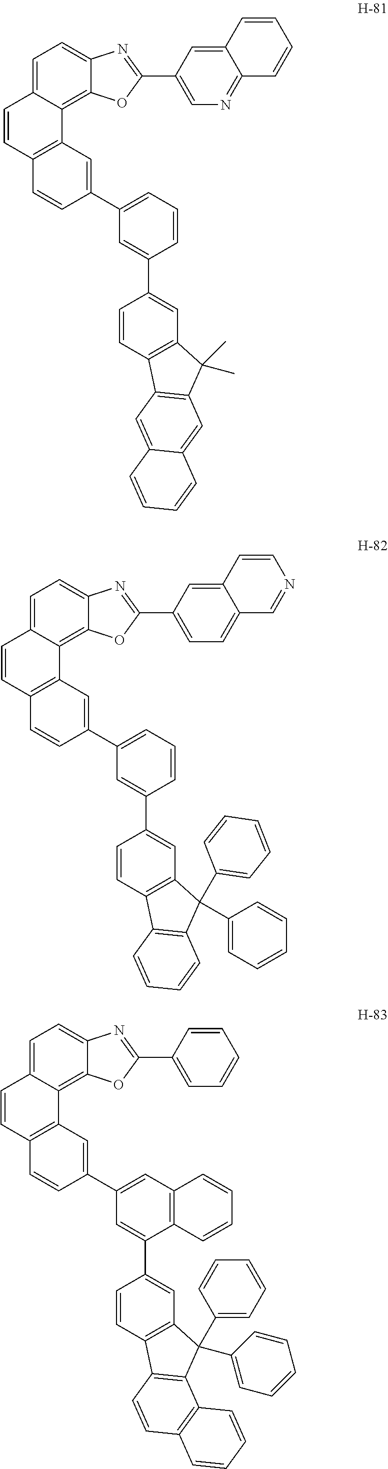

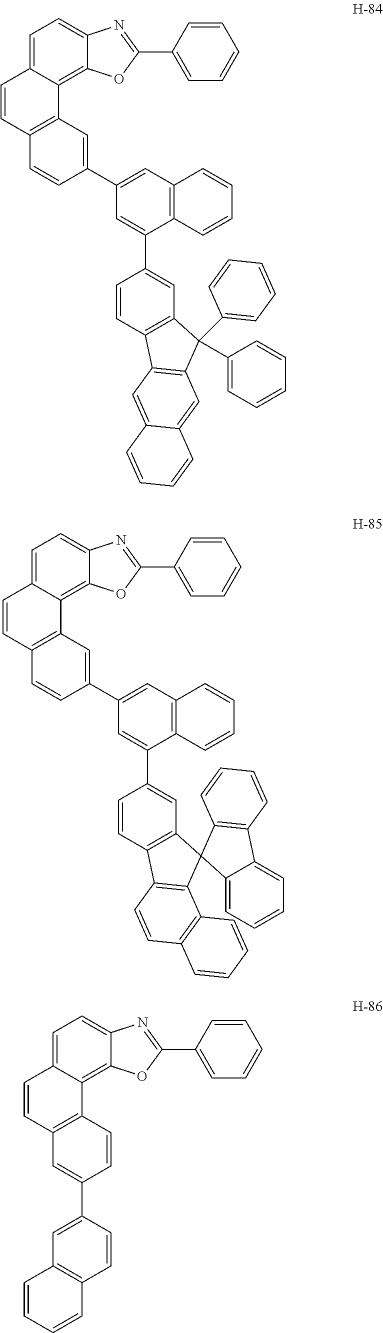

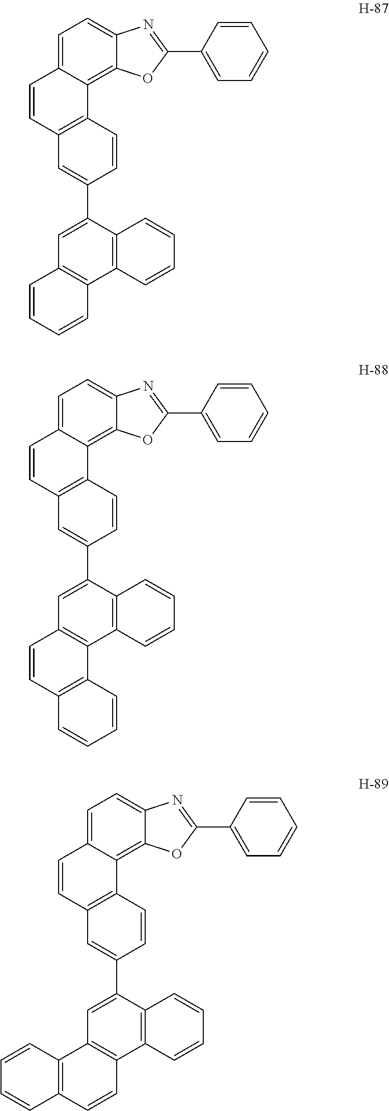 [SMILES: c1ccc(-c2nc3ccc4ccc5cc(-c6cc7ccccc7c7ccccc67)ccc5c4c3o2)cc1 ; c1ccc(-c2nc3ccc4ccc5cc(-c6cc7ccc8ccccc8c7c7ccccc67)ccc5c4c3o2)cc1 ; c1ccc(-c2nc3ccc4ccc5cc(-c6cc7c8ccccc8ccc7c7ccccc67)ccc5c4c3o2)cc1]

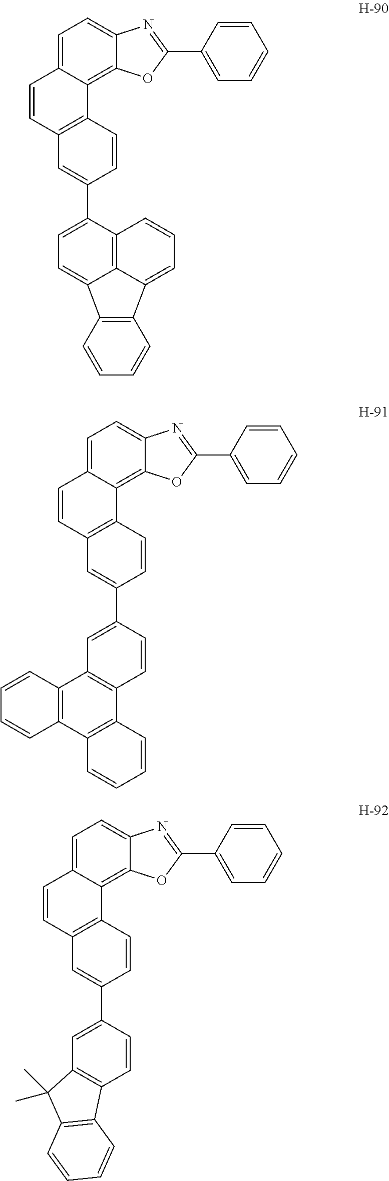

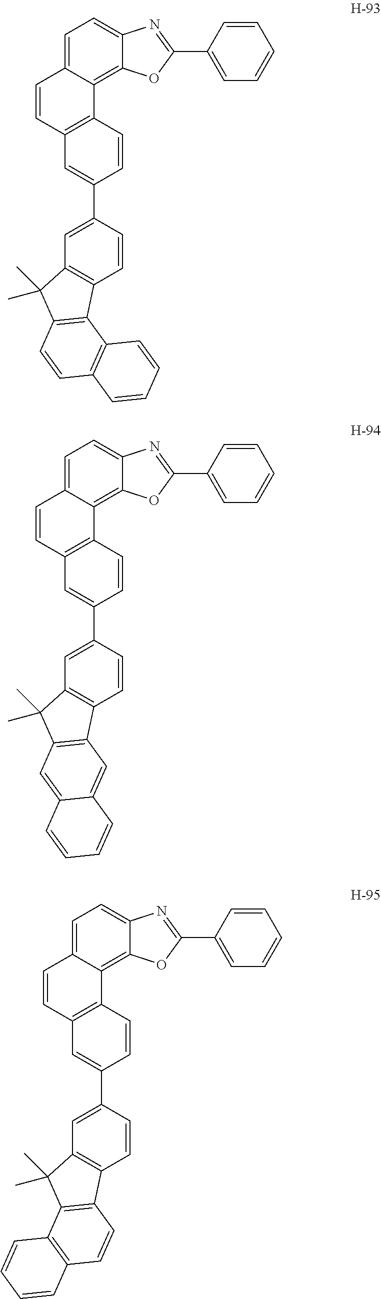

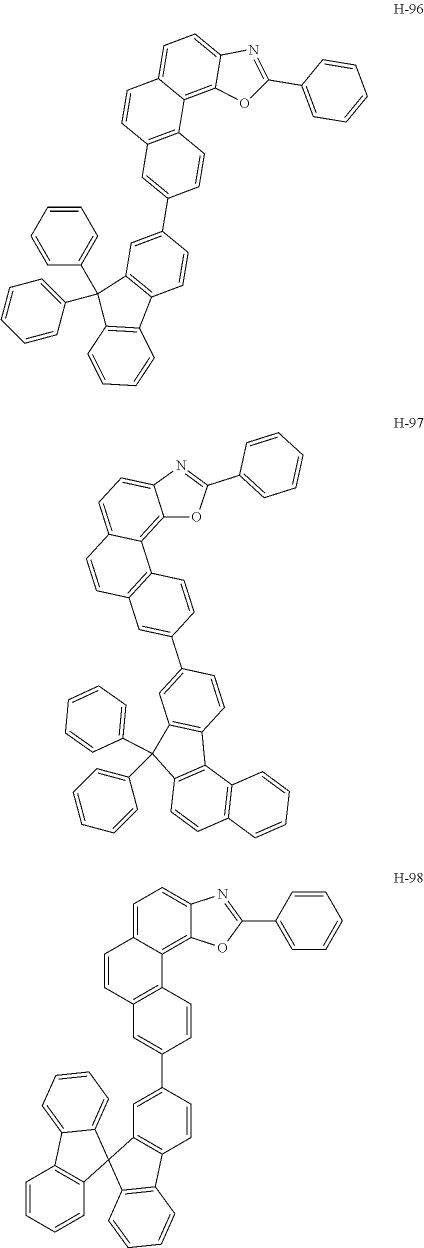 [SMILES: c1ccc(-c2nc3ccc4ccc5cc(-c6ccc7c(c6)C(c6ccccc6)(c6ccccc6)c6ccccc6-7)ccc5c4c3o2)cc1 ; c1ccc(-c2nc3ccc4ccc5cc(-c6ccc7c(c6)C(c6ccccc6)(c6ccccc6)c6ccc8ccccc8c6-7)ccc5c4c3o2)cc1 ; c1ccc(-c2nc3ccc4ccc5cc(-c6ccc7c(c6)C6(c8ccccc8-c8ccccc86)c6ccccc6-7)ccc5c4c3o2)cc1]

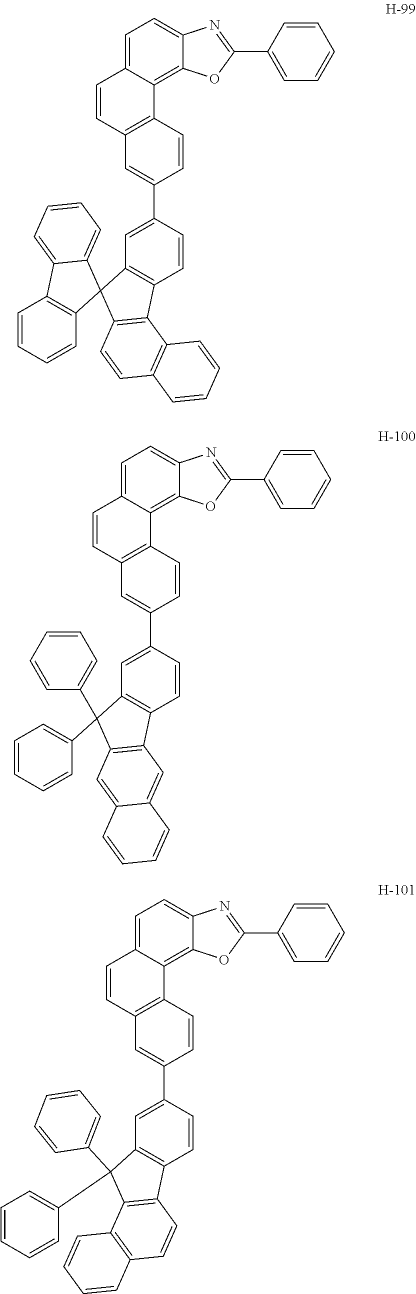

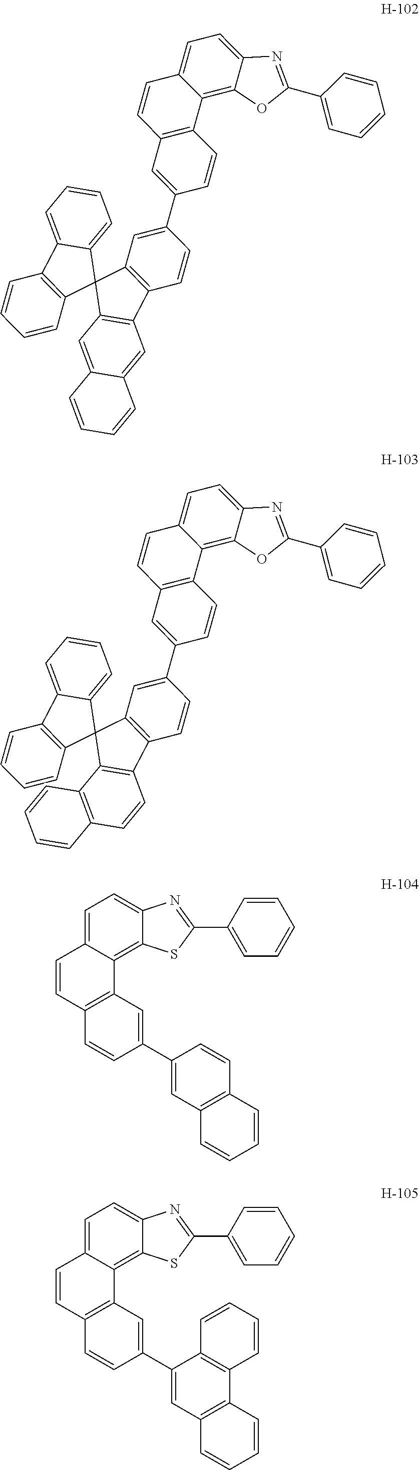 [SMILES: c1ccc(-c2nc3ccc4ccc5cc(-c6ccc7c(c6)C6(c8ccccc8-c8ccccc86)c6cc8ccccc8cc6-7)ccc5c4c3o2)cc1 ; c1ccc(-c2nc3ccc4ccc5cc(-c6ccc7c(c6)C6(c8ccccc8-c8ccccc86)c6c-7ccc7ccccc67)ccc5c4c3o2)cc1 ; c1ccc(-c2nc3ccc4ccc5ccc(-c6ccc7ccccc7c6)cc5c4c3s2)cc1 ; c1ccc(-c2nc3ccc4ccc5ccc(-c6cc7ccccc7c7ccccc67)cc5c4c3s2)cc1]

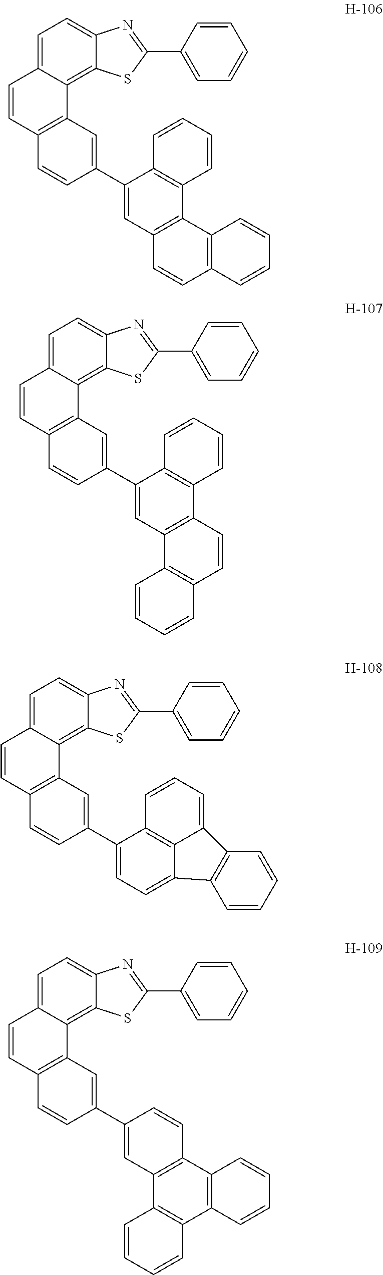

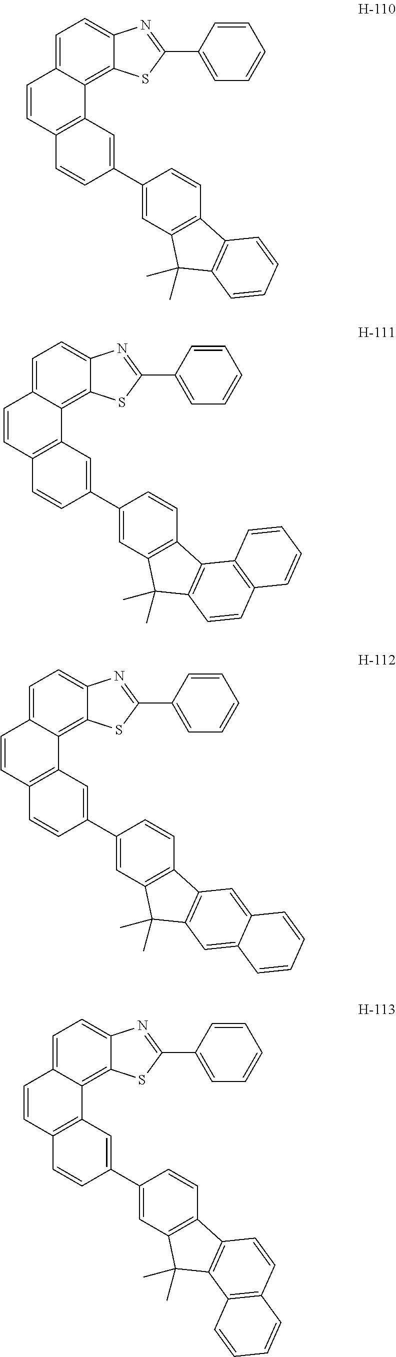 [SMILES: CC1(C)c2ccccc2-c2ccc(-c3ccc4ccc5ccc6nc(-c7ccccc7)sc6c5c4c3)cc21 ; CC1(C)c2cc(-c3ccc4ccc5ccc6nc(-c7ccccc7)sc6c5c4c3)ccc2-c2c1ccc1ccccc21 ; CC1(C)c2cc(-c3ccc4ccc5ccc6nc(-c7ccccc7)sc6c5c4c3)ccc2-c2cc3ccccc3cc21 ; CC1(C)c2cc(-c3ccc4ccc5ccc6nc(-c7ccccc7)sc6c5c4c3)ccc2-c2ccc3ccccc3c21]

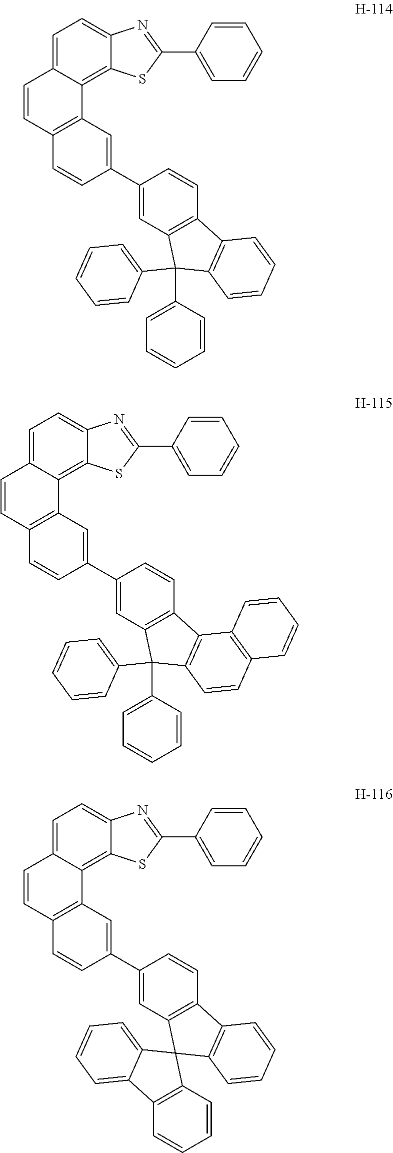 [SMILES: c1ccc(-c2nc3ccc4ccc5ccc(-c6ccc7c(c6)C(c6ccccc6)(c6ccccc6)c6ccccc6-7)cc5c4c3s2)cc1 ; c1ccc(-c2nc3ccc4ccc5ccc(-c6ccc7c(c6)C(c6ccccc6)(c6ccccc6)c6ccc8ccccc8c6-7)cc5c4c3s2)cc1 ; c1ccc(-c2nc3ccc4ccc5ccc(-c6ccc7c(c6)C6(c8ccccc8-c8ccccc86)c6ccccc6-7)cc5c4c3s2)cc1]

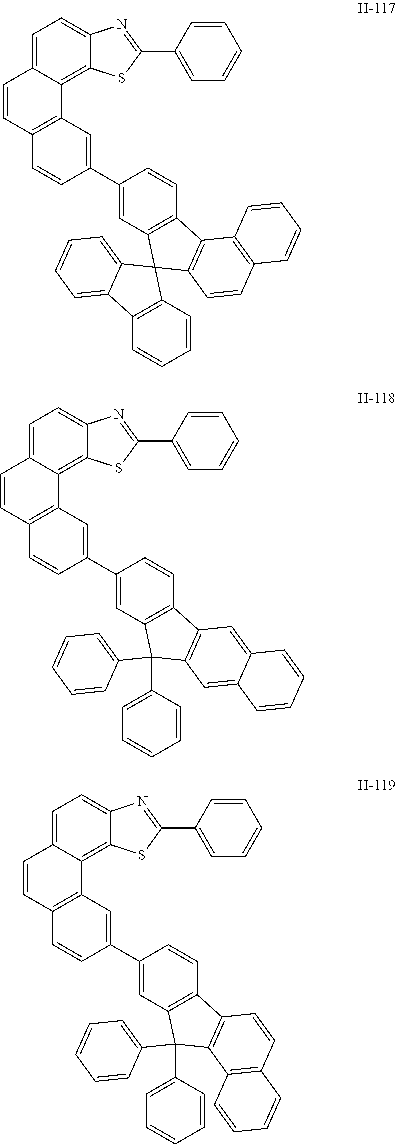

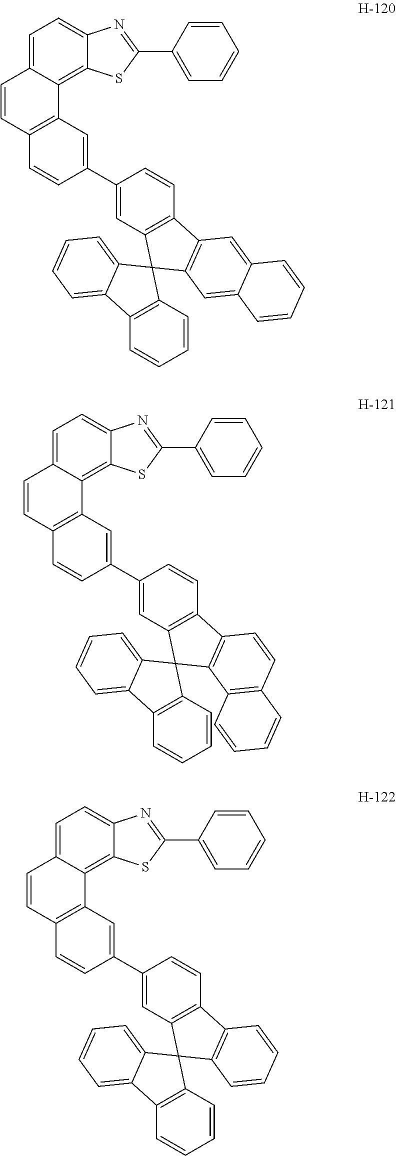 [SMILES: c1ccc(-c2nc3ccc4ccc5ccc(-c6ccc7c(c6)C6(c8ccccc8-c8ccccc86)c6cc8ccccc8cc6-7)cc5c4c3s2)cc1 ; c1ccc(-c2nc3ccc4ccc5ccc(-c6ccc7c(c6)C6(c8ccccc8-c8ccccc86)c6c-7ccc7ccccc67)cc5c4c3s2)cc1 ; c1ccc(-c2nc3ccc4ccc5ccc(-c6ccc7c(c6)C6(c8ccccc8-c8ccccc86)c6ccccc6-7)cc5c4c3s2)cc1]

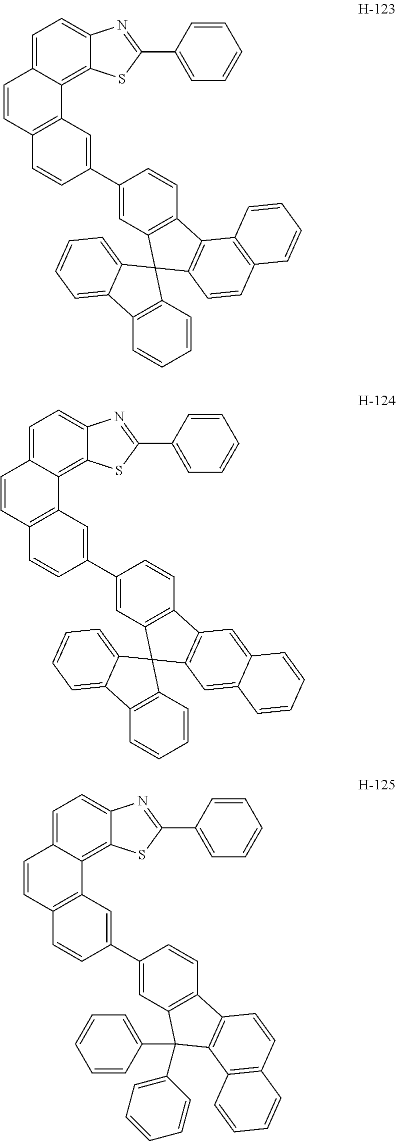

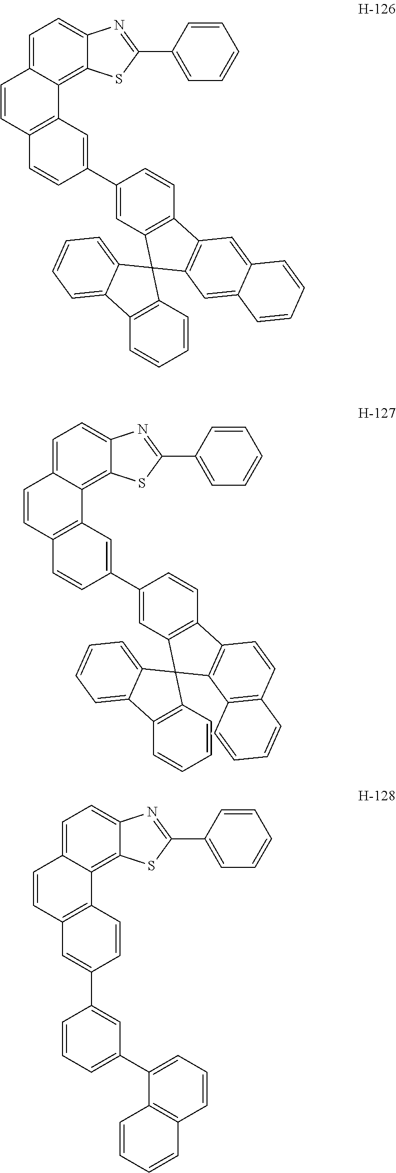 [SMILES: c1ccc(-c2nc3ccc4ccc5ccc(-c6ccc7c(c6)C6(c8ccccc8-c8ccccc86)c6cc8ccccc8cc6-7)cc5c4c3s2)cc1 ; c1ccc(-c2nc3ccc4ccc5ccc(-c6ccc7c(c6)C6(c8ccccc8-c8ccccc86)c6c-7ccc7ccccc67)cc5c4c3s2)cc1 ; c1ccc(-c2nc3ccc4ccc5cc(-c6cccc(-c7cccc8ccccc78)c6)ccc5c4c3s2)cc1]

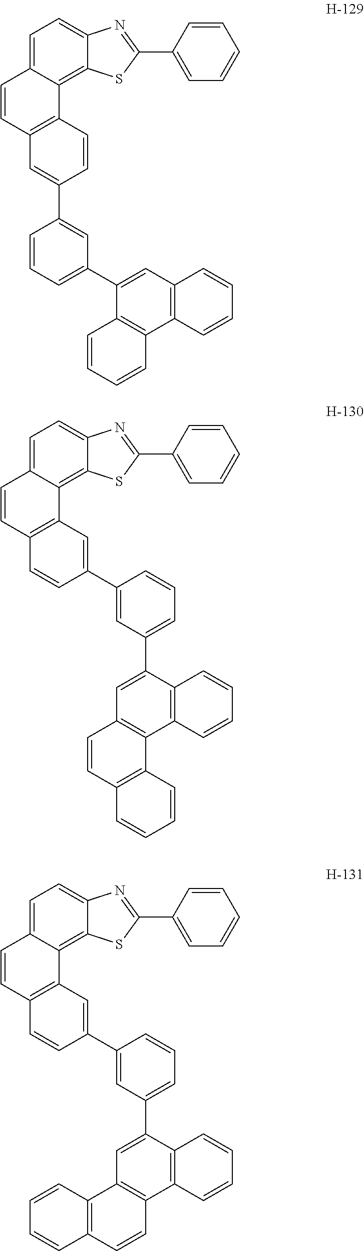

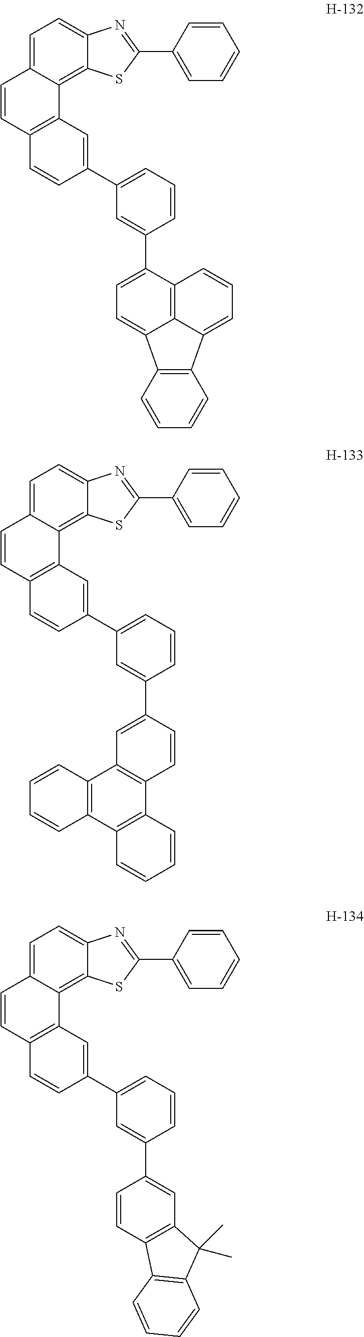

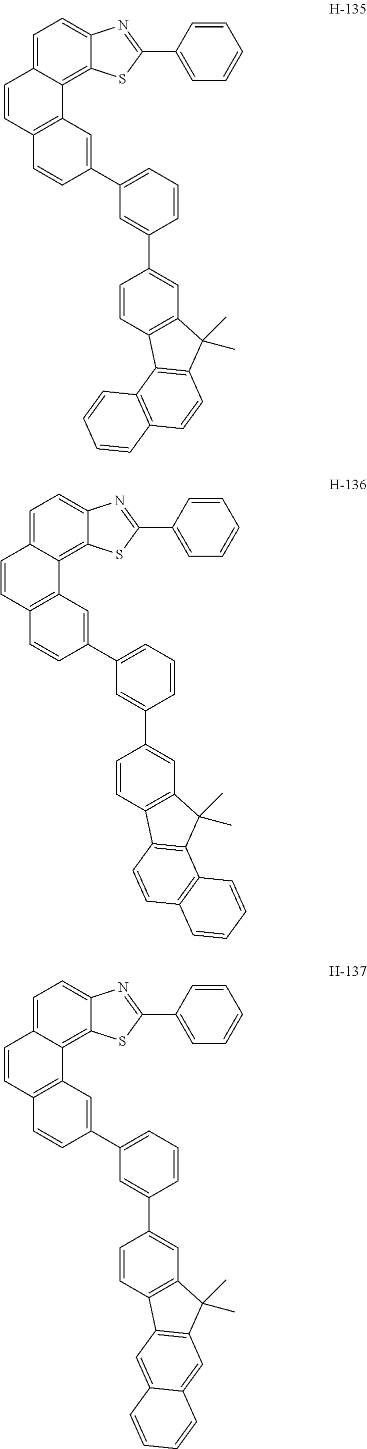 [SMILES: CC1(C)c2cc(-c3cccc(-c4ccc5ccc6ccc7nc(-c8ccccc8)sc7c6c5c4)c3)ccc2-c2c1ccc1ccccc21 ; CC1(C)c2cc(-c3cccc(-c4ccc5ccc6ccc7nc(-c8ccccc8)sc7c6c5c4)c3)ccc2-c2ccc3ccccc3c21 ; CC1(C)c2cc(-c3cccc(-c4ccc5ccc6ccc7nc(-c8ccccc8)sc7c6c5c4)c3)ccc2-c2cc3ccccc3cc21]

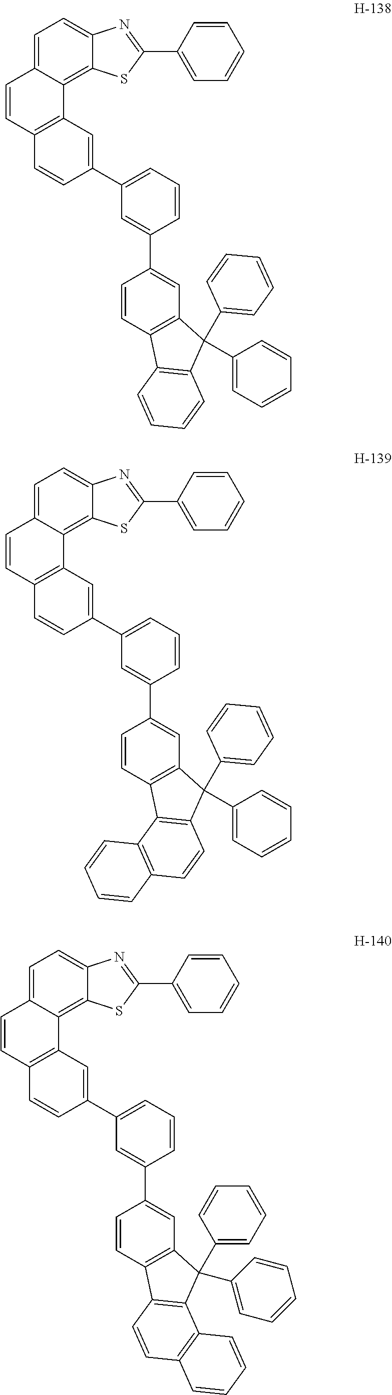

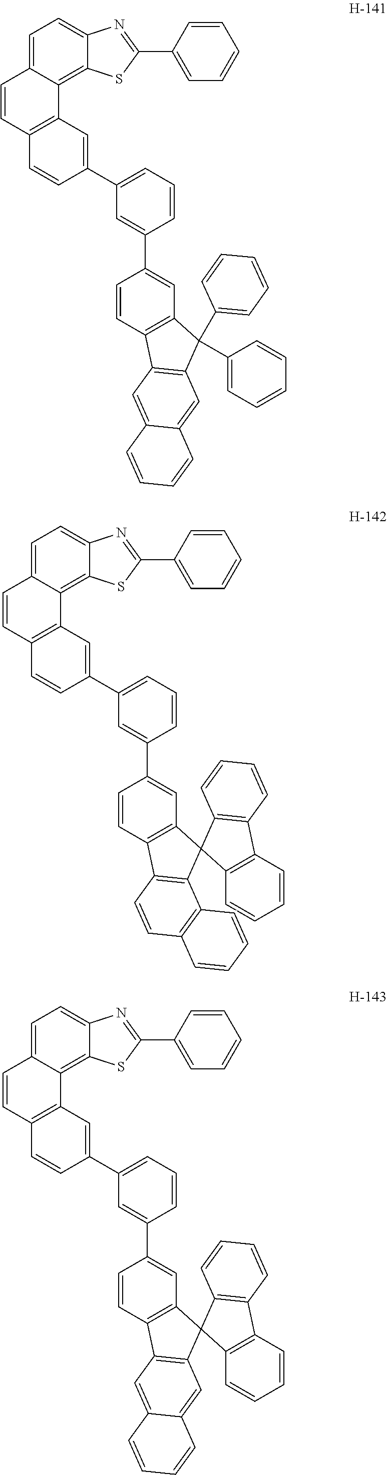

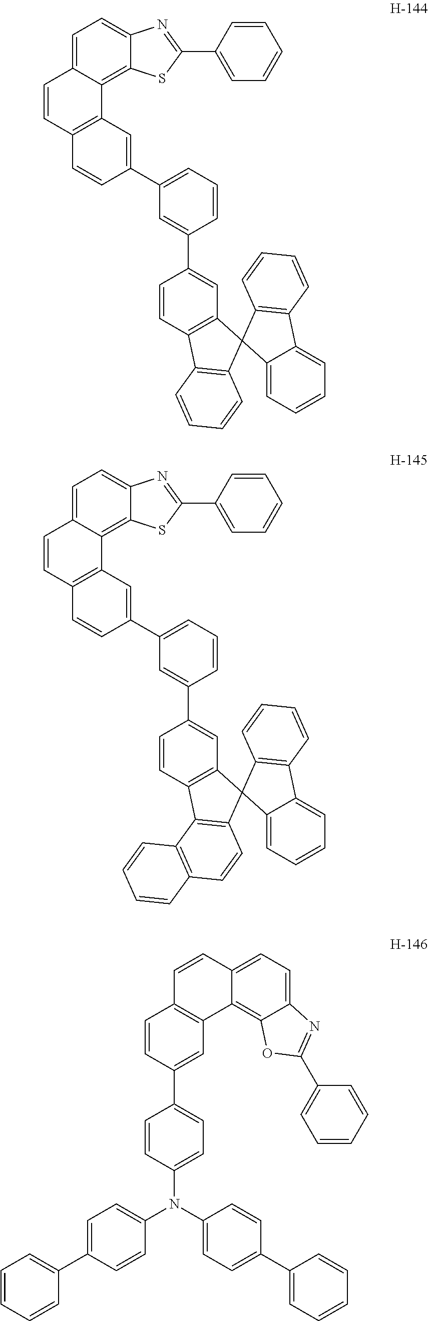 [SMILES: c1ccc(-c2nc3ccc4ccc5ccc(-c6cccc(-c7ccc8c(c7)C7(c9ccccc9-c9ccccc97)c7ccccc7-8)c6)cc5c4c3s2)cc1 ; c1ccc(-c2nc3ccc4ccc5ccc(-c6cccc(-c7ccc8c(c7)C7(c9ccccc9-c9ccccc97)c7ccc9ccccc9c7-8)c6)cc5c4c3s2)cc1 ; c1ccc(-c2ccc(N(c3ccc(-c4ccccc4)cc3)c3ccc(-c4ccc5ccc6ccc7nc(-c8ccccc8)oc7c6c5c4)cc3)cc2)cc1]

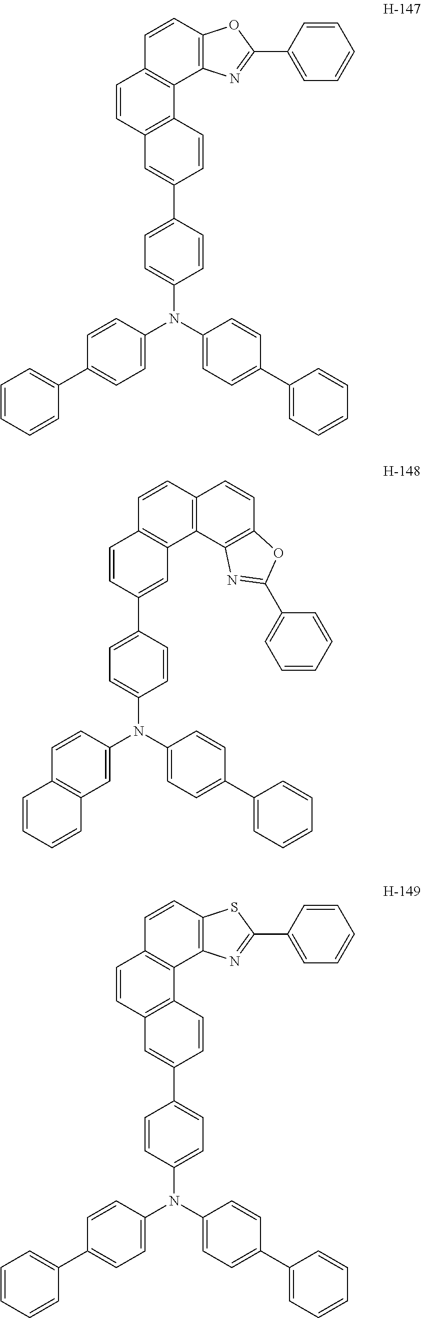 [SMILES: c1ccc(-c2ccc(N(c3ccc(-c4ccccc4)cc3)c3ccc(-c4ccc5c(ccc6ccc7oc(-c8ccccc8)nc7c65)c4)cc3)cc2)cc1 ; c1ccc(-c2ccc(N(c3ccc(-c4ccc5ccc6ccc7oc(-c8ccccc8)nc7c6c5c4)cc3)c3ccc4ccccc4c3)cc2)cc1 ; c1ccc(-c2ccc(N(c3ccc(-c4ccccc4)cc3)c3ccc(-c4ccc5c(ccc6ccc7sc(-c8ccccc8)nc7c65)c4)cc3)cc2)cc1]

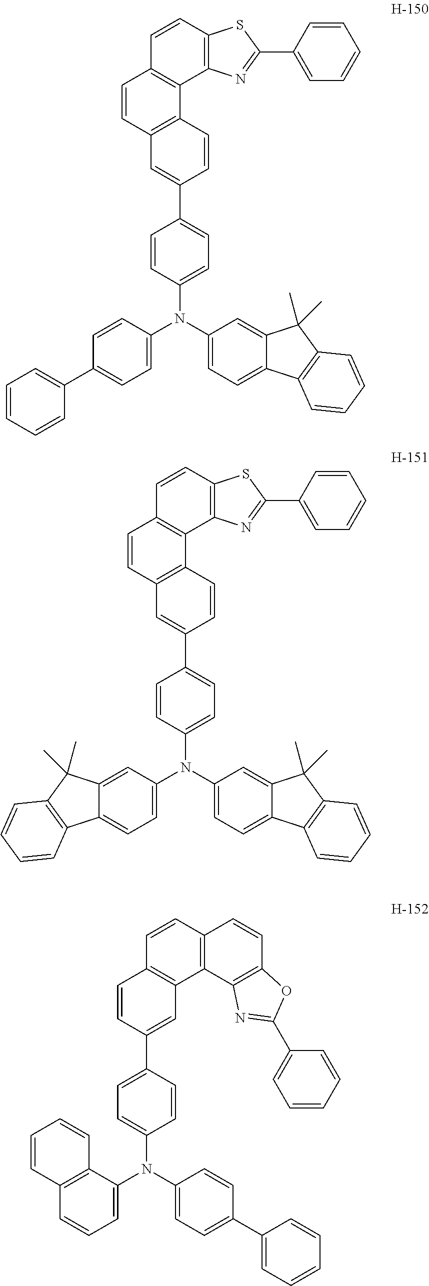 [SMILES: CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21 ; CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21 ; c1ccc(-c2ccc(N(c3ccc(-c4ccc5ccc6ccc7oc(-c8ccccc8)nc7c6c5c4)cc3)c3cccc4ccccc34)cc2)cc1]

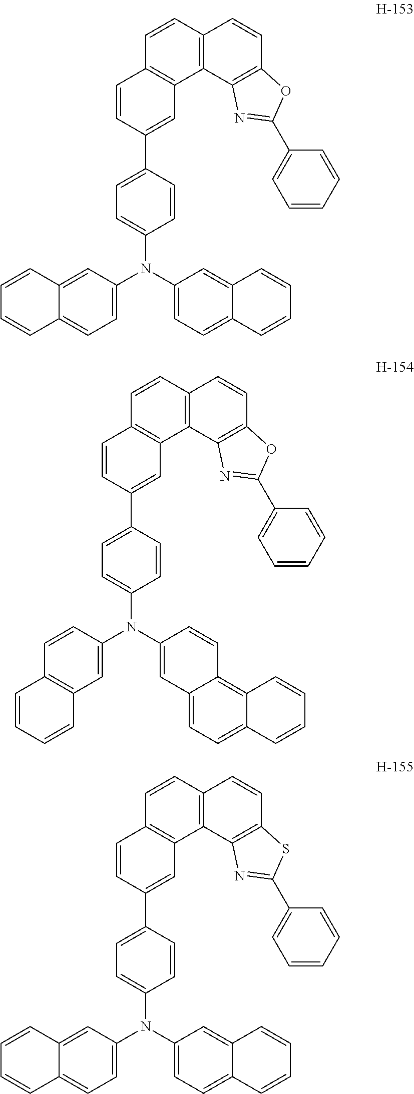

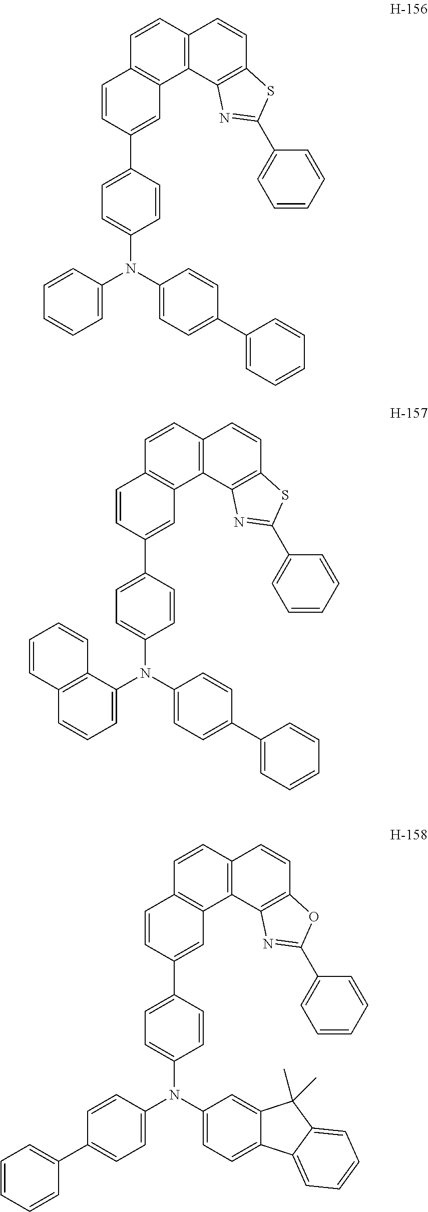 [SMILES: c1ccc(-c2ccc(N(c3ccccc3)c3ccc(-c4ccc5ccc6ccc7sc(-c8ccccc8)nc7c6c5c4)cc3)cc2)cc1 ; c1ccc(-c2ccc(N(c3ccc(-c4ccc5ccc6ccc7sc(-c8ccccc8)nc7c6c5c4)cc3)c3cccc4ccccc34)cc2)cc1 ; CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21]

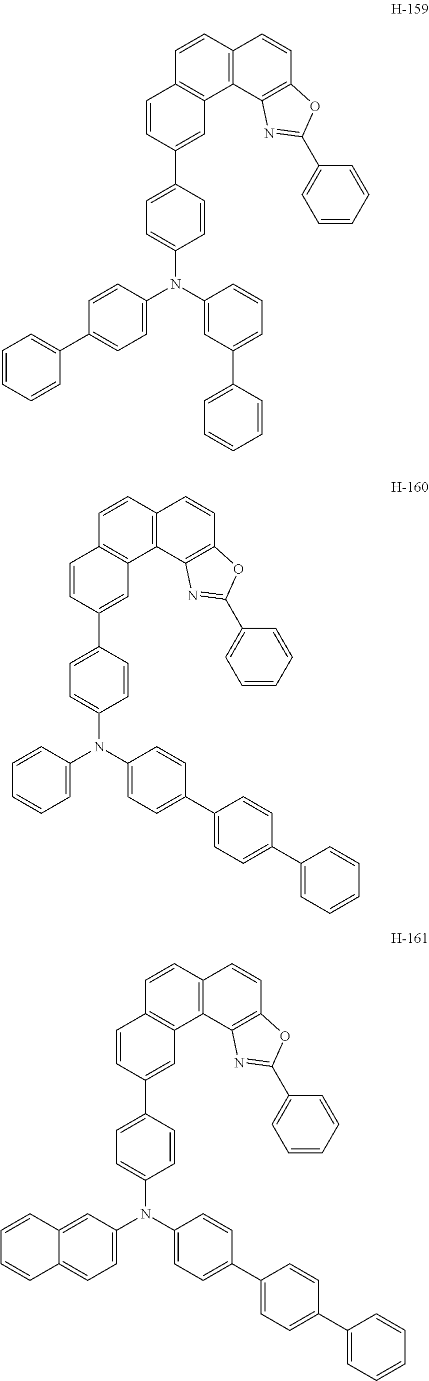 [SMILES: c1ccc(-c2ccc(N(c3ccc(-c4ccc5ccc6ccc7oc(-c8ccccc8)nc7c6c5c4)cc3)c3cccc(-c4ccccc4)c3)cc2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1]

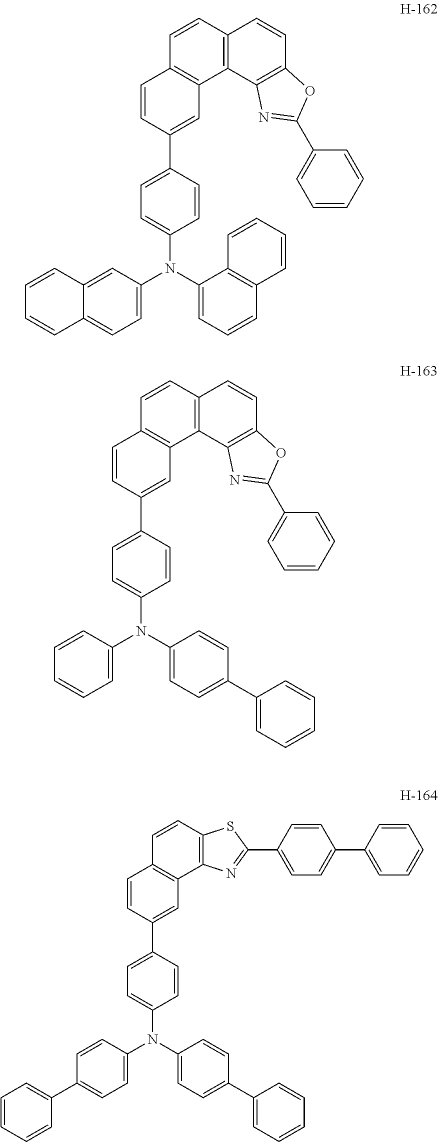

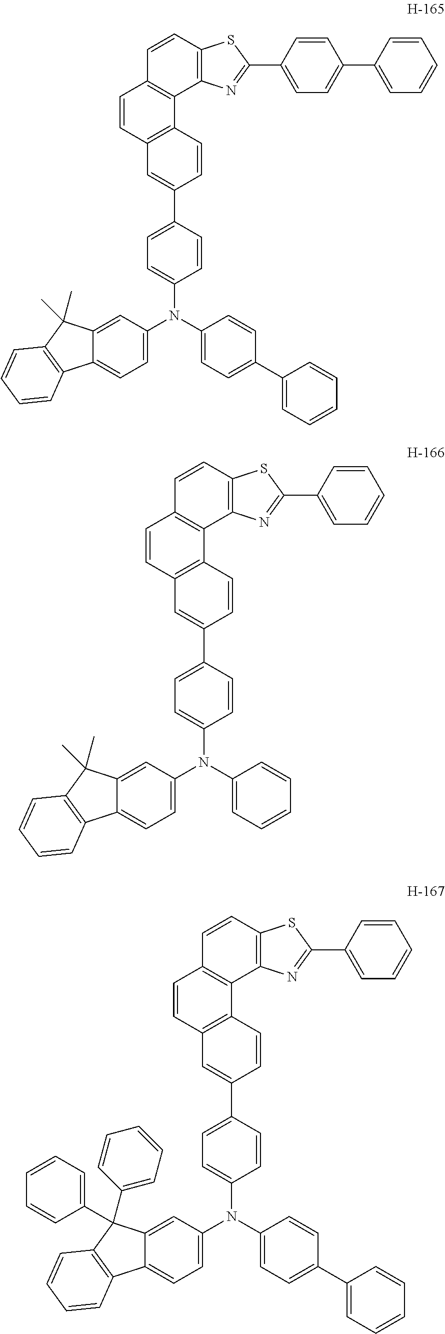 [SMILES: CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21 ; CC1(C)c2ccccc2-c2ccc(N(c3ccccc3)c3ccc(-c4ccc5c(ccc6ccc7sc(-c8ccccc8)nc7c65)c4)cc3)cc21 ; c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1]

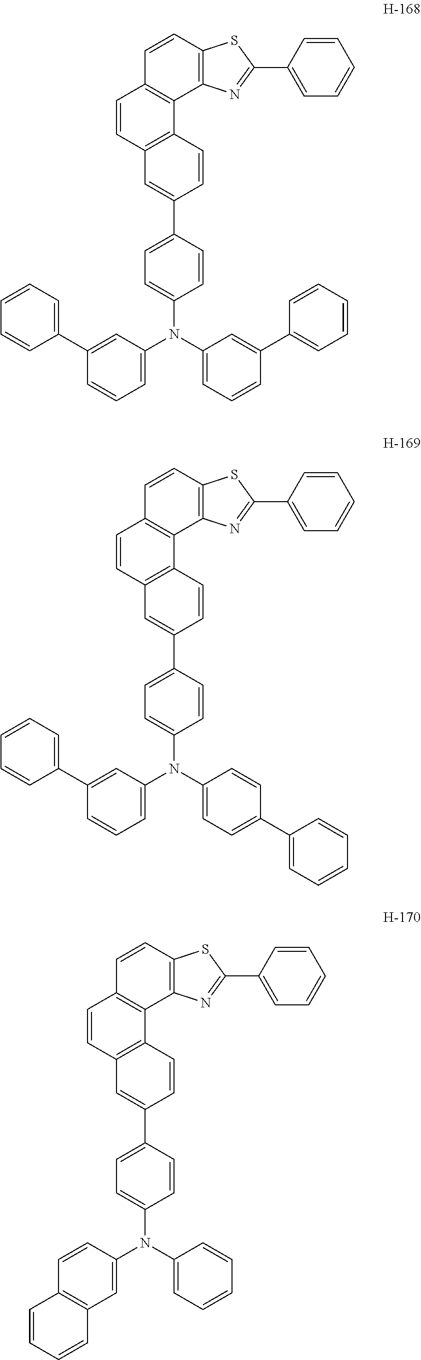 [SMILES: c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3cccc(-c4ccccc4)c3)cc2)cc1 ; c1ccc(-c2nc3c(ccc4ccc5cc(-c6ccc(N(c7ccccc7)c7ccc8ccccc8c7)cc6)ccc5c43)s2)cc1]

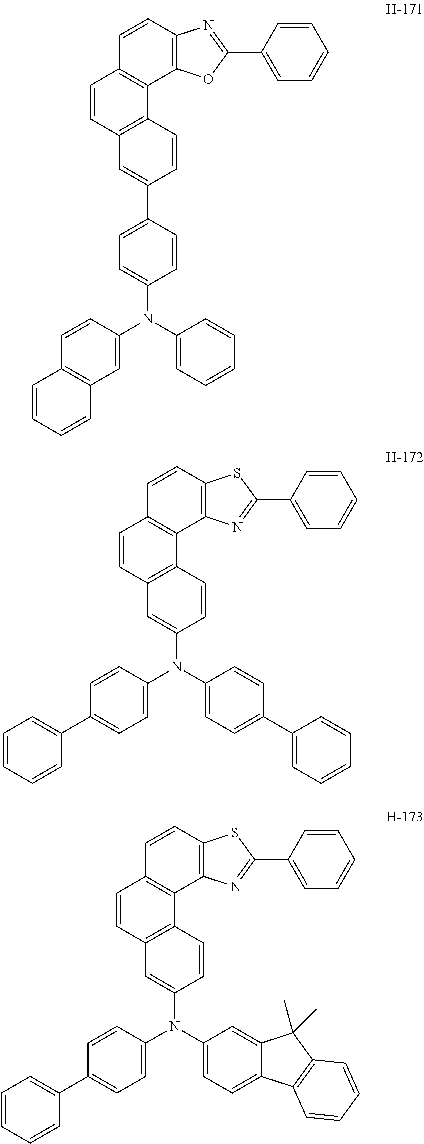 [SMILES: c1ccc(-c2nc3ccc4ccc5cc(-c6ccc(N(c7ccccc7)c7ccc8ccccc8c7)cc6)ccc5c4c3o2)cc1 ; c1ccc(-c2ccc(N(c3ccc(-c4ccccc4)cc3)c3ccc4c(ccc5ccc6sc(-c7ccccc7)nc6c54)c3)cc2)cc1 ; CC1(C)c2ccccc2-c2ccc(N(c3ccc(-c4ccccc4)cc3)c3ccc4c(ccc5ccc6sc(-c7ccccc7)nc6c54)c3)cc21]

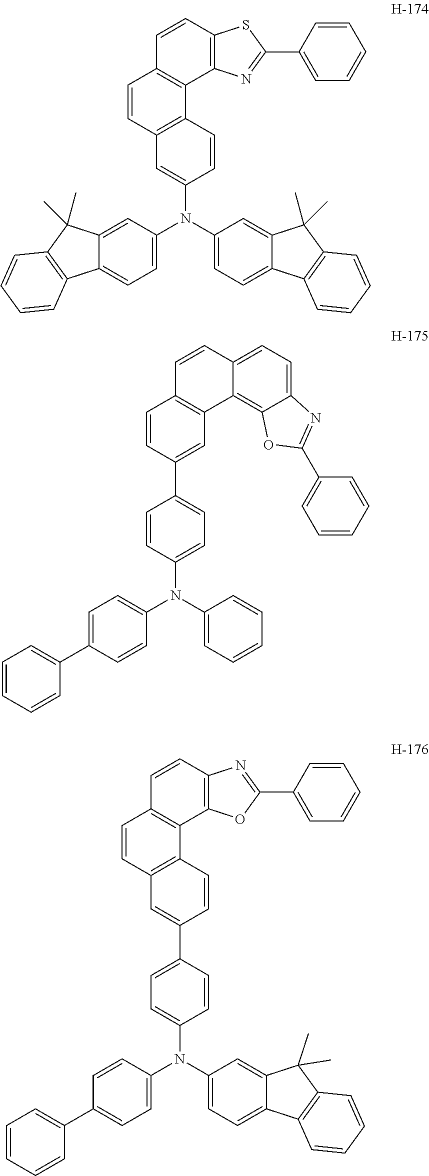 [SMILES: CC1(C)c2ccccc2-c2ccc(N(c3ccc4c(c3)C(C)(C)c3ccccc3-4)c3ccc4c(ccc5ccc6sc(-c7ccccc7)nc6c54)c3)cc21 ; c1ccc(-c2ccc(N(c3ccccc3)c3ccc(-c4ccc5ccc6ccc7nc(-c8ccccc8)oc7c6c5c4)cc3)cc2)cc1 ; CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21]

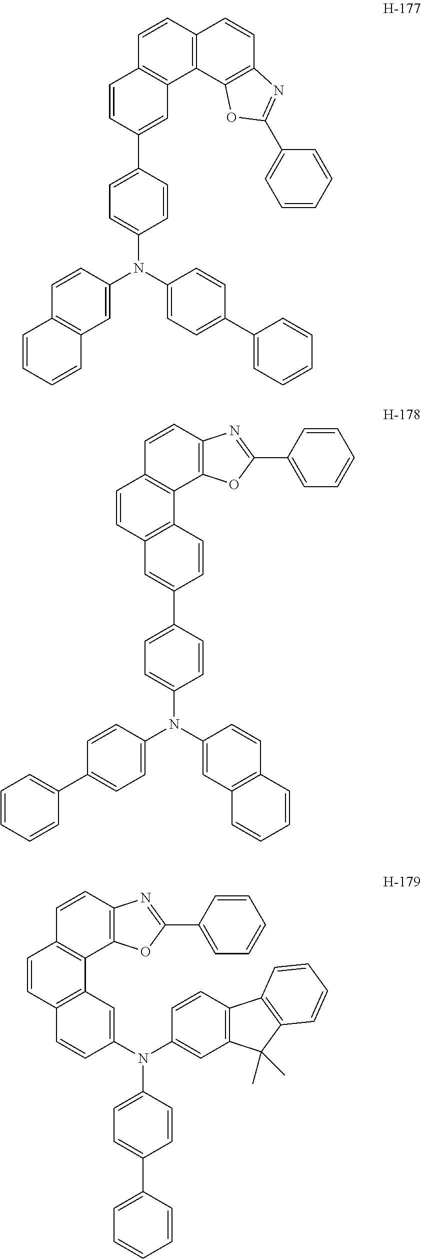 [SMILES: c1ccc(-c2ccc(N(c3ccc(-c4ccc5ccc6ccc7nc(-c8ccccc8)oc7c6c5c4)cc3)c3ccc4ccccc4c3)cc2)cc1 ; c1ccc(-c2ccc(N(c3ccc(-c4ccc5c(ccc6ccc7nc(-c8ccccc8)oc7c65)c4)cc3)c3ccc4ccccc4c3)cc2)cc1 ; CC1(C)c2ccccc2-c2ccc(N(c3ccc(-c4ccccc4)cc3)c3ccc4ccc5ccc6nc(-c7ccccc7)oc6c5c4c3)cc21]

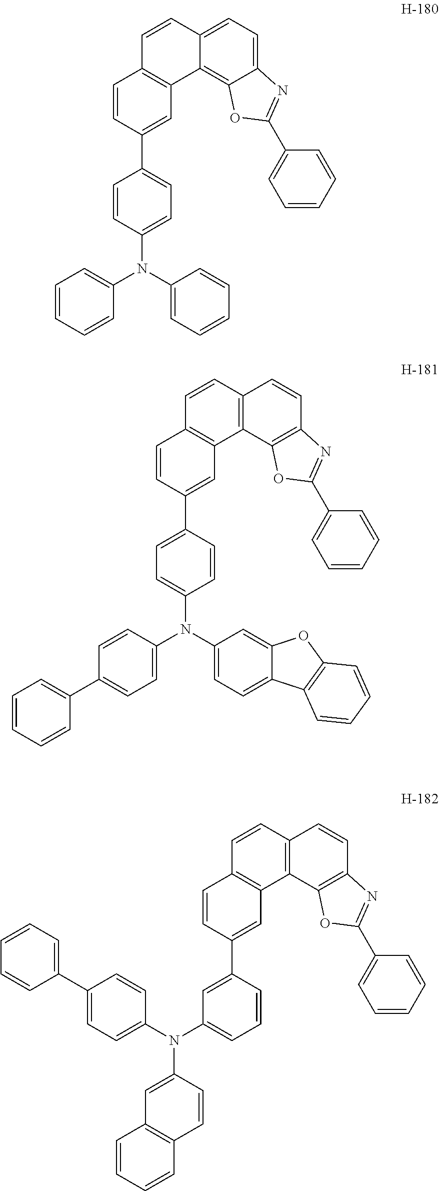

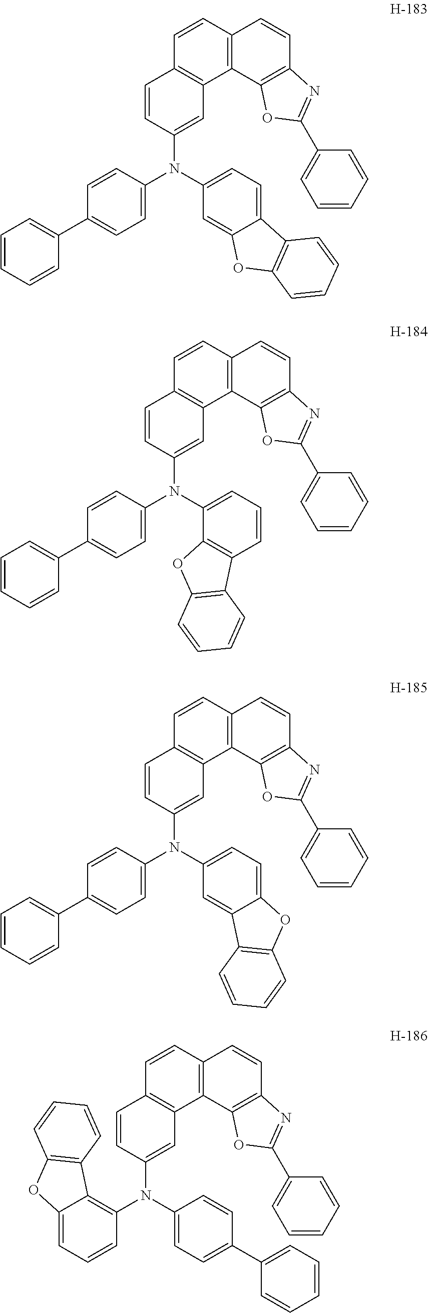

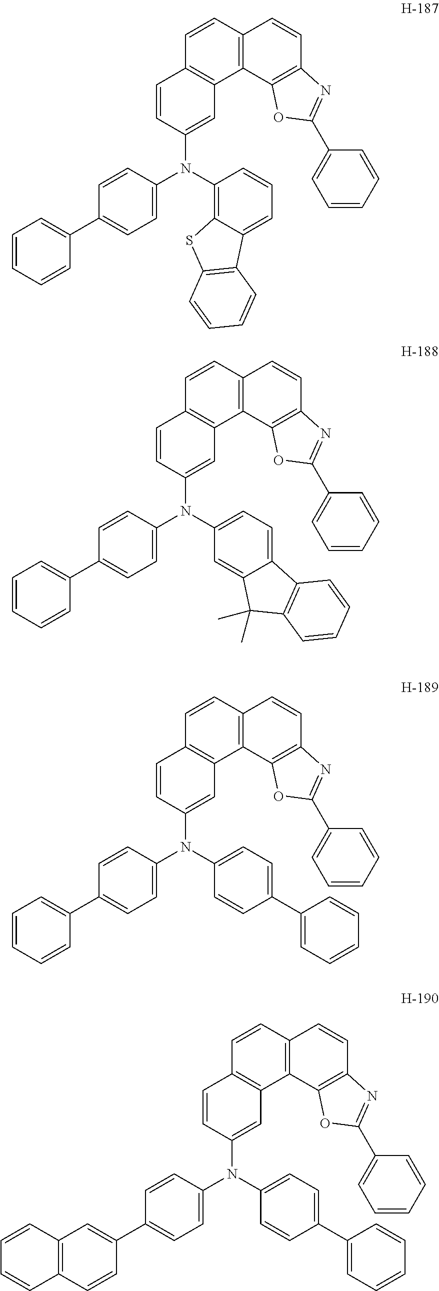

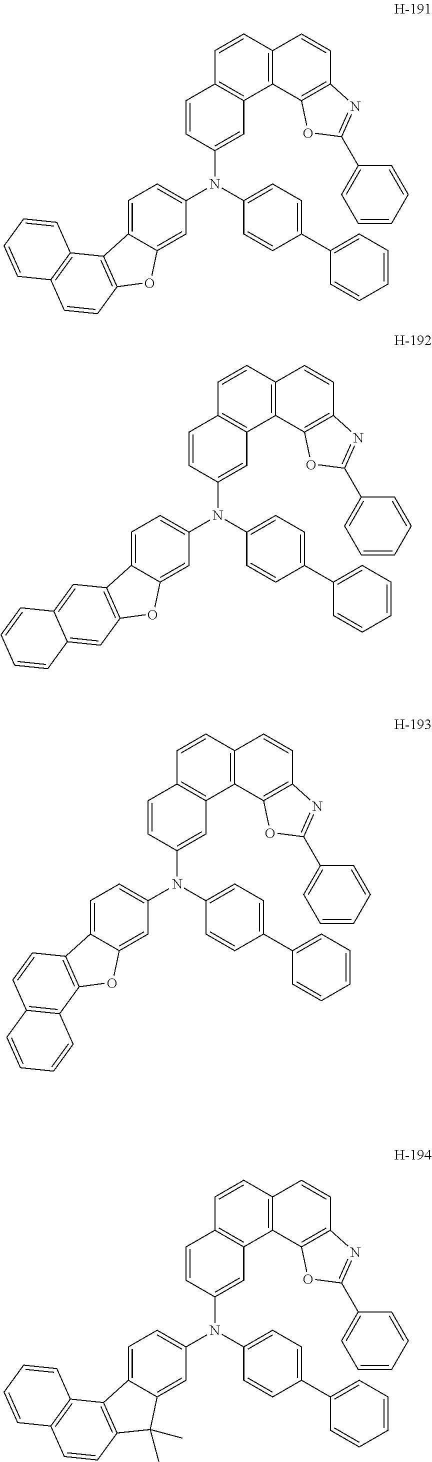 [SMILES: c1ccc(-c2ccc(N(c3ccc4c(c3)oc3ccc5ccccc5c34)c3ccc4ccc5ccc6nc(-c7ccccc7)oc6c5c4c3)cc2)cc1 ; c1ccc(-c2ccc(N(c3ccc4c(c3)oc3cc5ccccc5cc34)c3ccc4ccc5ccc6nc(-c7ccccc7)oc6c5c4c3)cc2)cc1 ; c1ccc(-c2ccc(N(c3ccc4c(c3)oc3c5ccccc5ccc43)c3ccc4ccc5ccc6nc(-c7ccccc7)oc6c5c4c3)cc2)cc1 ; CC1(C)c2cc(N(c3ccc(-c4ccccc4)cc3)c3ccc4ccc5ccc6nc(-c7ccccc7)oc6c5c4c3)ccc2-c2c1ccc1ccccc21]

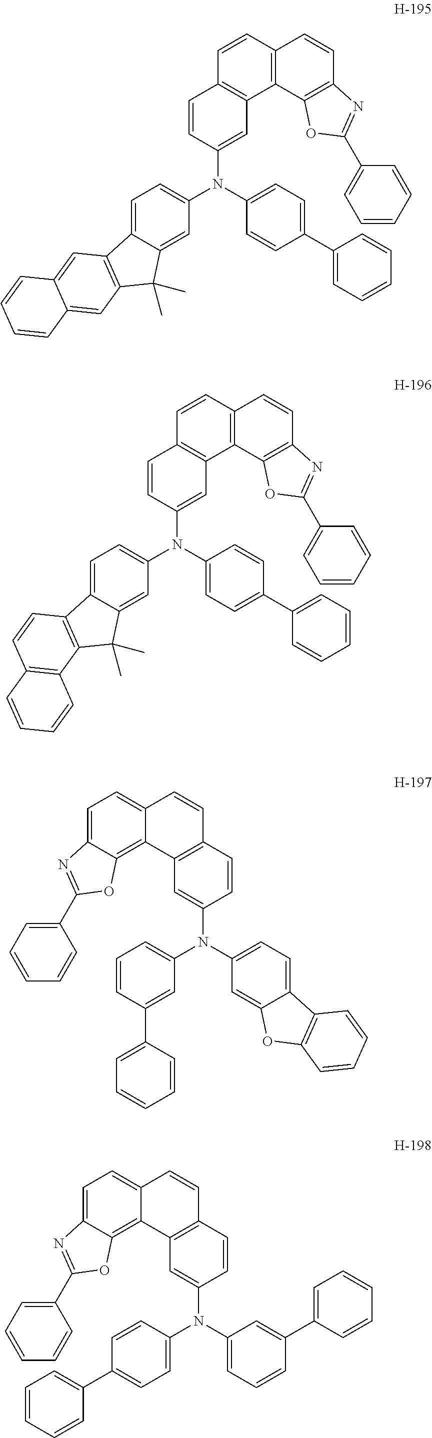 [SMILES: CC1(C)c2cc(N(c3ccc(-c4ccccc4)cc3)c3ccc4ccc5ccc6nc(-c7ccccc7)oc6c5c4c3)ccc2-c2cc3ccccc3cc21 ; CC1(C)c2cc(N(c3ccc(-c4ccccc4)cc3)c3ccc4ccc5ccc6nc(-c7ccccc7)oc6c5c4c3)ccc2-c2ccc3ccccc3c21 ; c1ccc(-c2cccc(N(c3ccc4c(c3)oc3ccccc34)c3ccc4ccc5ccc6nc(-c7ccccc7)oc6c5c4c3)c2)cc1 ; c1ccc(-c2ccc(N(c3cccc(-c4ccccc4)c3)c3ccc4ccc5ccc6nc(-c7ccccc7)oc6c5c4c3)cc2)cc1]

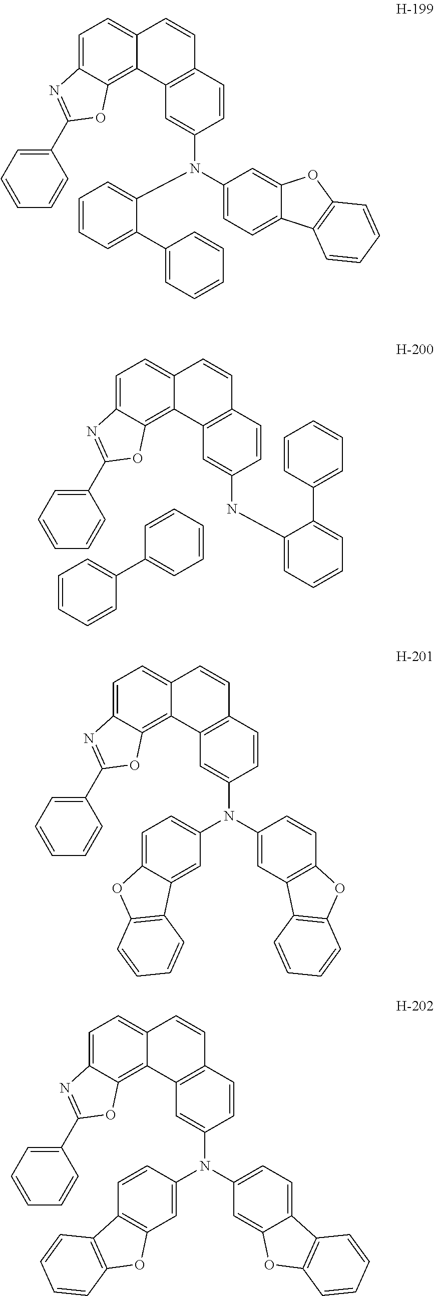

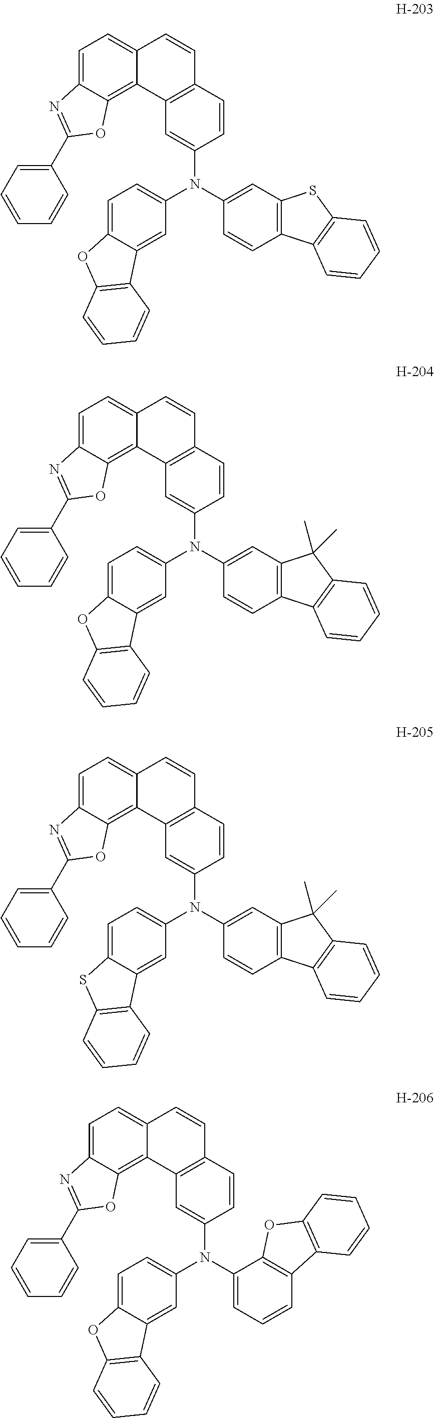

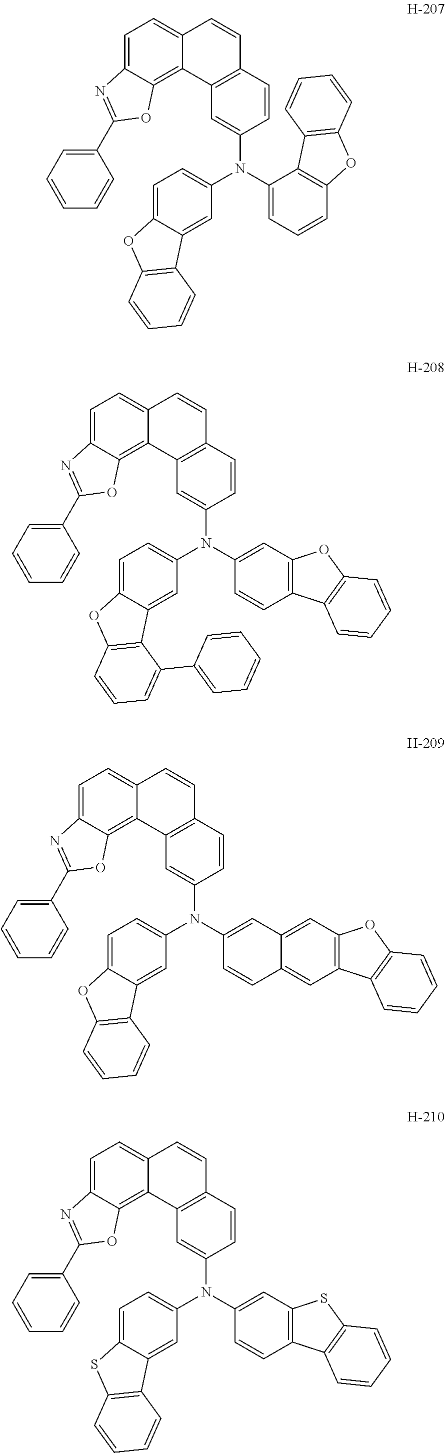 [SMILES: c1ccc(-c2nc3ccc4ccc5ccc(N(c6ccc7oc8ccccc8c7c6)c6cccc7oc8ccccc8c67)cc5c4c3o2)cc1 ; c1ccc(-c2nc3ccc4ccc5ccc(N(c6ccc7c(c6)oc6ccccc67)c6ccc7oc8cccc(-c9ccccc9)c8c7c6)cc5c4c3o2)cc1 ; c1ccc(-c2nc3ccc4ccc5ccc(N(c6ccc7cc8c(cc7c6)oc6ccccc68)c6ccc7oc8ccccc8c7c6)cc5c4c3o2)cc1 ; c1ccc(-c2nc3ccc4ccc5ccc(N(c6ccc7c(c6)sc6ccccc67)c6ccc7sc8ccccc8c7c6)cc5c4c3o2)cc1]

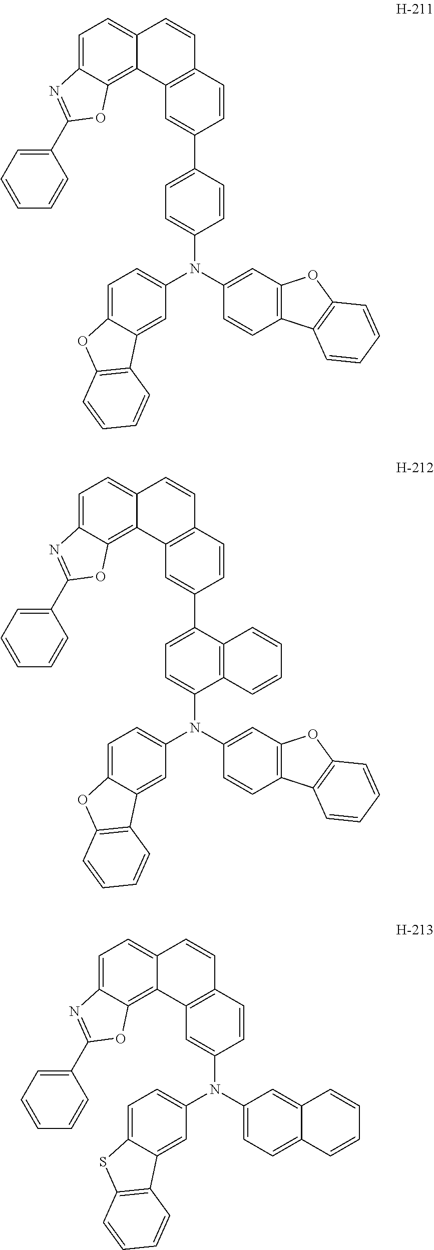 [SMILES: c1ccc(-c2nc3ccc4ccc5ccc(-c6ccc(N(c7ccc8c(c7)oc7ccccc78)c7ccc8oc9ccccc9c8c7)cc6)cc5c4c3o2)cc1 ; c1ccc(-c2nc3ccc4ccc5ccc(-c6ccc(N(c7ccc8c(c7)oc7ccccc78)c7ccc8oc9ccccc9c8c7)c7ccccc67)cc5c4c3o2)cc1 ; c1ccc(-c2nc3ccc4ccc5ccc(N(c6ccc7ccccc7c6)c6ccc7sc8ccccc8c7c6)cc5c4c3o2)cc1]

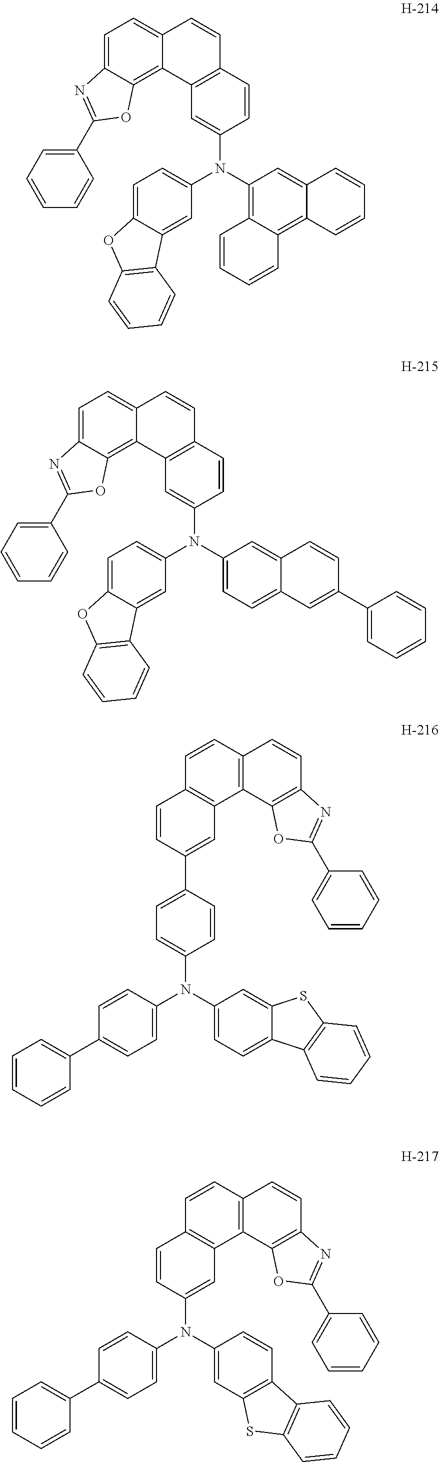 [SMILES: c1ccc(-c2nc3ccc4ccc5ccc(N(c6ccc7oc8ccccc8c7c6)c6cc7ccccc7c7ccccc67)cc5c4c3o2)cc1 ; c1ccc(-c2ccc3cc(N(c4ccc5oc6ccccc6c5c4)c4ccc5ccc6ccc7nc(-c8ccccc8)oc7c6c5c4)ccc3c2)cc1 ; c1ccc(-c2ccc(N(c3ccc(-c4ccc5ccc6ccc7nc(-c8ccccc8)oc7c6c5c4)cc3)c3ccc4c(c3)sc3ccccc34)cc2)cc1 ; c1ccc(-c2ccc(N(c3ccc4c(c3)sc3ccccc34)c3ccc4ccc5ccc6nc(-c7ccccc7)oc6c5c4c3)cc2)cc1]

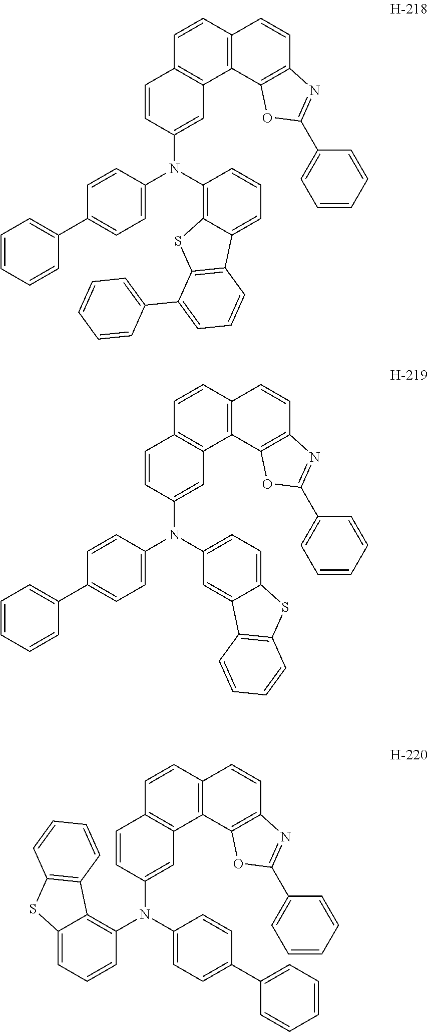 [SMILES: c1ccc(-c2ccc(N(c3ccc4ccc5ccc6nc(-c7ccccc7)oc6c5c4c3)c3cccc4c3sc3c(-c5ccccc5)cccc34)cc2)cc1 ; c1ccc(-c2ccc(N(c3ccc4sc5ccccc5c4c3)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3cccc4sc5ccccc5c34)cc2)cc1]

The compound represented by formula 3 may be at least one selected from the following compounds, but is not limited thereto.

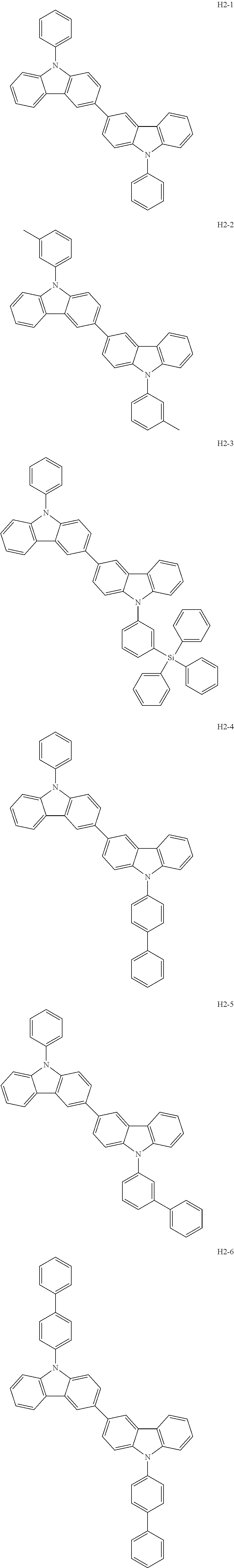 [SMILES: c1ccc(-n2c3ccccc3c3cc(-c4ccc5c(c4)c4ccccc4n5-c4ccccc4)ccc32)cc1 ; Cc1cccc(-n2c3ccccc3c3cc(-c4ccc5c(c4)c4ccccc4n5-c4cccc(C)c4)ccc32)c1 ; c1ccc(-n2c3ccccc3c3cc(-c4ccc5c(c4)c4ccccc4n5-c4cccc([Si](c5ccccc5)(c5ccccc5)c5ccccc5)c4)ccc32)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

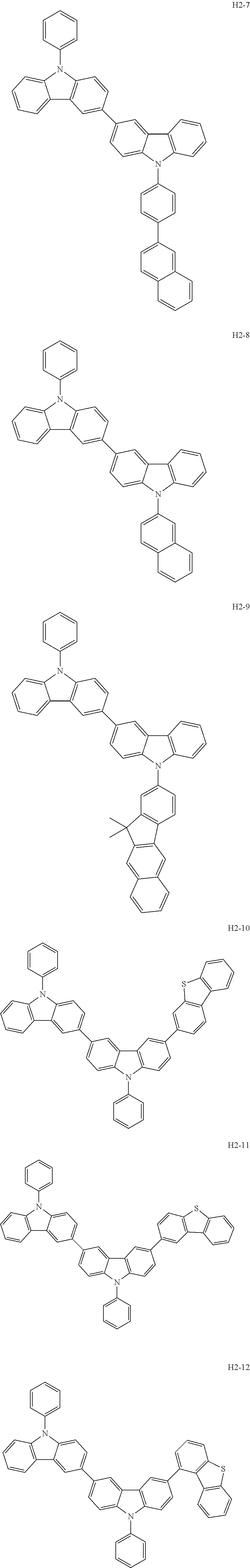

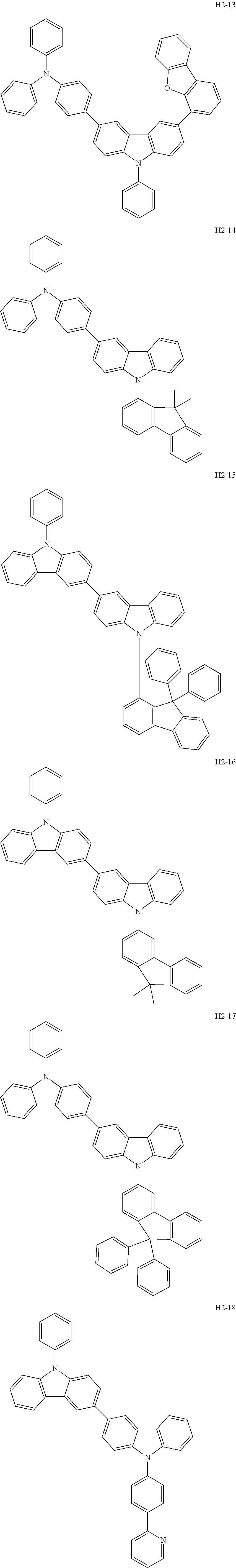

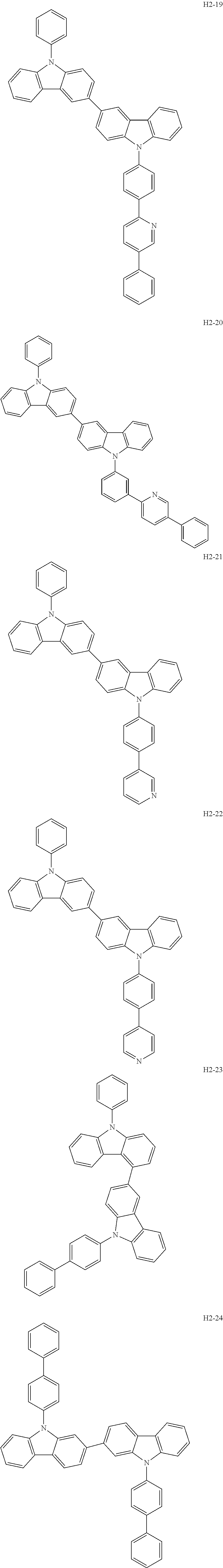 [SMILES: c1ccc(-c2ccc(-c3ccc(-n4c5ccccc5c5cc(-c6ccc7c(c6)c6ccccc6n7-c6ccccc6)ccc54)cc3)nc2)cc1 ; c1ccc(-c2ccc(-c3cccc(-n4c5ccccc5c5cc(-c6ccc7c(c6)c6ccccc6n7-c6ccccc6)ccc54)c3)nc2)cc1 ; c1ccc(-n2c3ccccc3c3cc(-c4ccc5c(c4)c4ccccc4n5-c4ccc(-c5cccnc5)cc4)ccc32)cc1 ; c1ccc(-n2c3ccccc3c3cc(-c4ccc5c(c4)c4ccccc4n5-c4ccc(-c5ccncc5)cc4)ccc32)cc1 ; c1ccc(-c2ccc(-n3c4ccccc4c4cc(-c5cccc6c5c5ccccc5n6-c5ccccc5)ccc43)cc2)cc1 ; c1ccc(-c2ccc(-n3c4ccccc4c4ccc(-c5ccc6c7ccccc7n(-c7ccc(-c8ccccc8)cc7)c6c5)cc43)cc2)cc1]

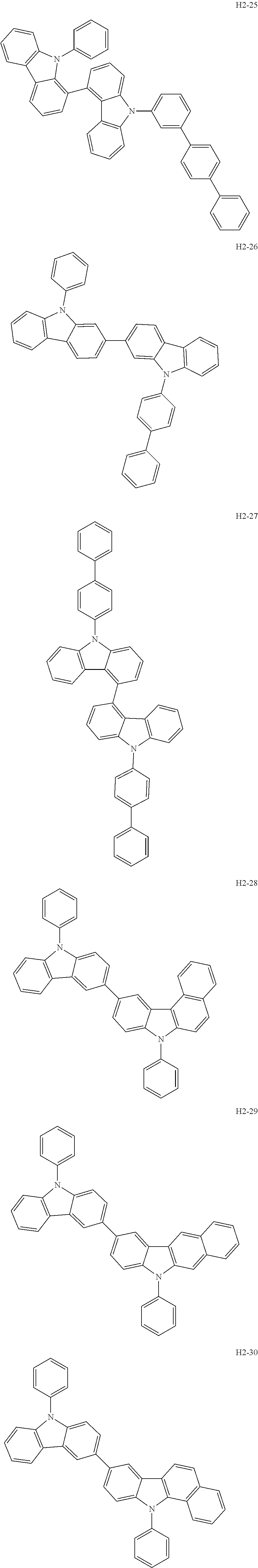 [SMILES: c1ccc(-c2ccc(-c3cccc(-n4c5ccccc5c5c(-c6cccc7c8ccccc8n(-c8ccccc8)c67)cccc54)c3)cc2)cc1 ; c1ccc(-c2ccc(-n3c4ccccc4c4ccc(-c5ccc6c7ccccc7n(-c7ccccc7)c6c5)cc43)cc2)cc1 ; c1ccc(-c2ccc(-n3c4ccccc4c4c(-c5cccc6c5c5ccccc5n6-c5ccc(-c6ccccc6)cc5)cccc43)cc2)cc1 ; c1ccc(-n2c3ccccc3c3cc(-c4ccc5c(c4)c4c6ccccc6ccc4n5-c4ccccc4)ccc32)cc1 ; c1ccc(-n2c3ccccc3c3cc(-c4ccc5c(c4)c4cc6ccccc6cc4n5-c4ccccc4)ccc32)cc1 ; c1ccc(-n2c3ccccc3c3cc(-c4ccc5c(c4)c4ccc6ccccc6c4n5-c4ccccc4)ccc32)cc1]

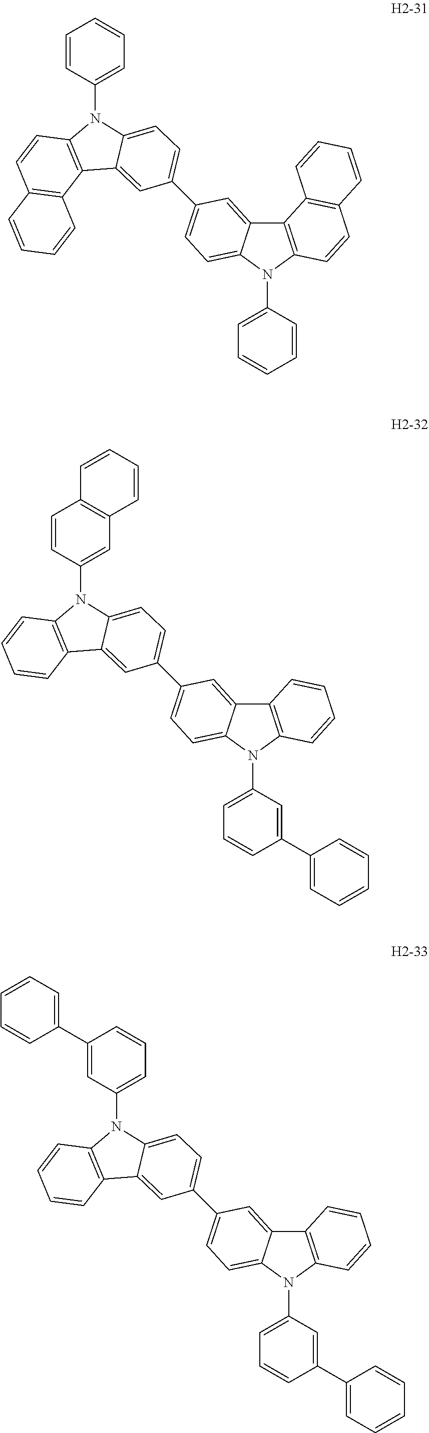 [SMILES: c1ccc(-n2c3ccc(-c4ccc5c(c4)c4c6ccccc6ccc4n5-c4ccccc4)cc3c3c4ccccc4ccc32)cc1 ; c1ccc(-c2cccc(-n3c4ccccc4c4cc(-c5ccc6c(c5)c5ccccc5n6-c5ccc6ccccc6c5)ccc43)c2)cc1 ; c1ccc(-c2cccc(-n3c4ccccc4c4cc(-c5ccc6c(c5)c5ccccc5n6-c5cccc(-c6ccccc6)c5)ccc43)c2)cc1]

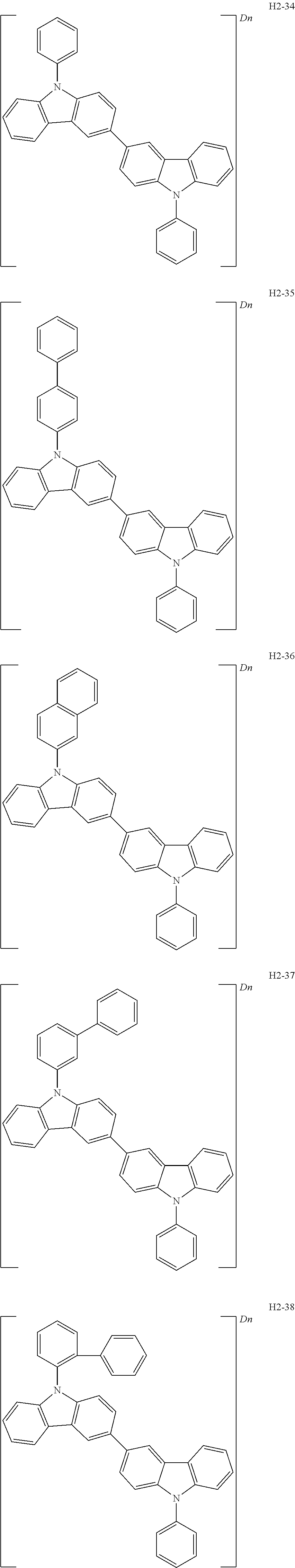

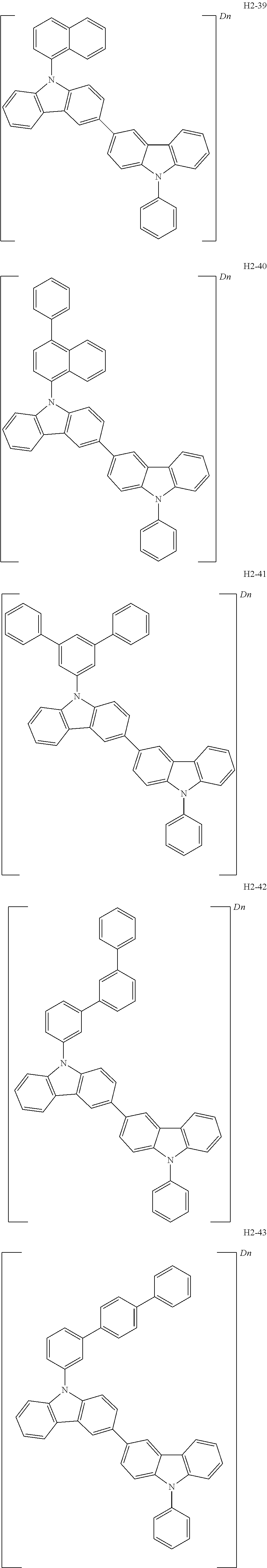

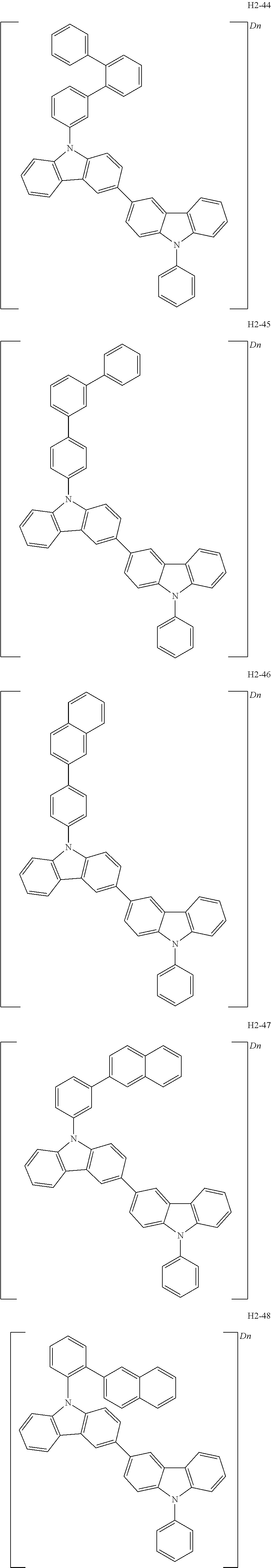

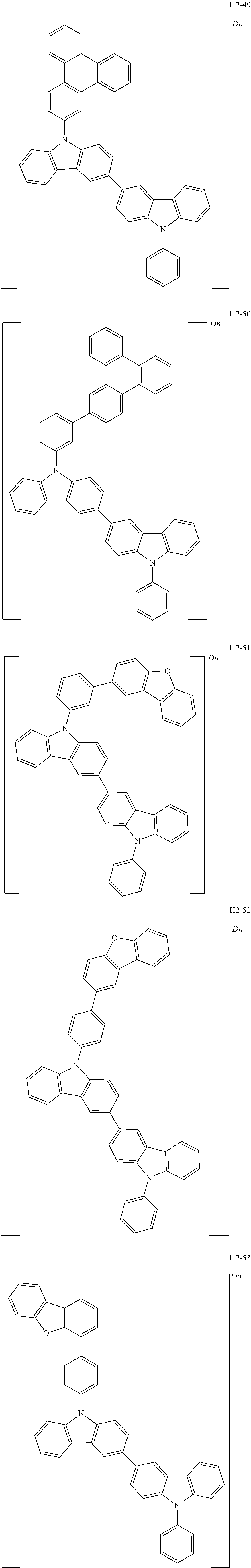

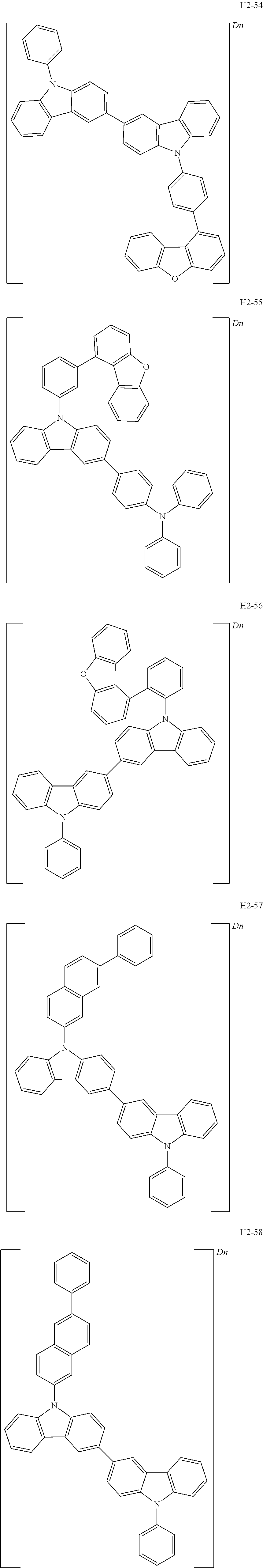

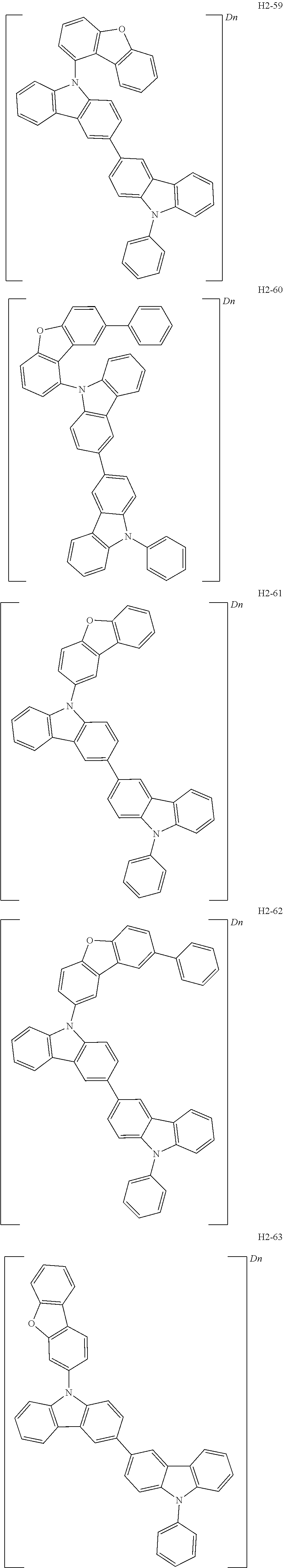

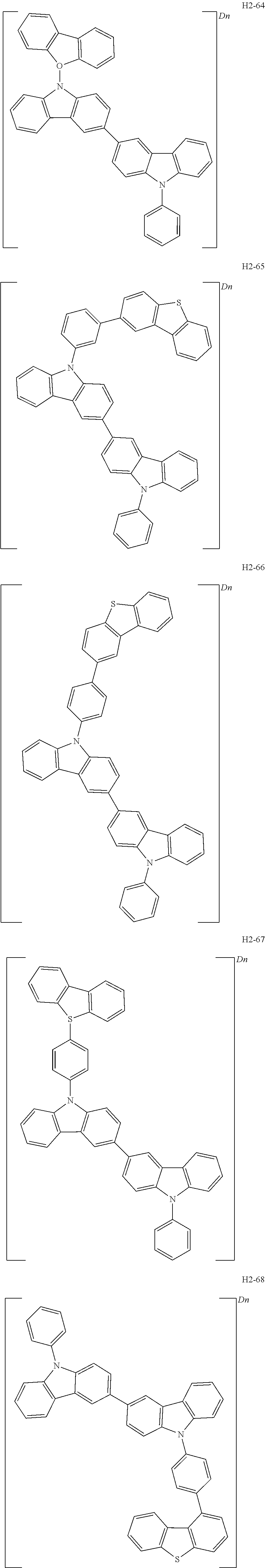

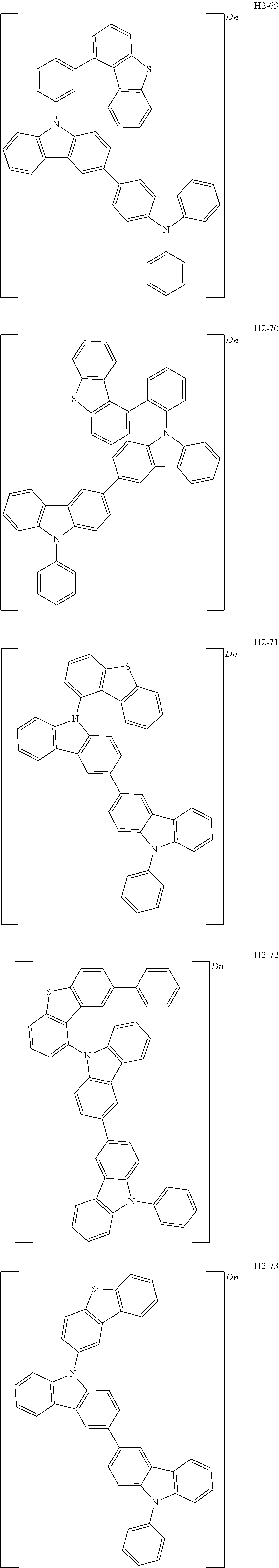

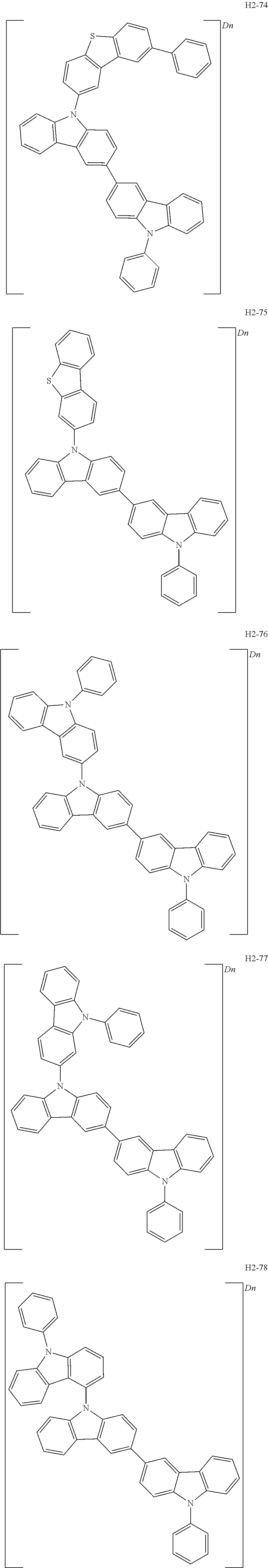

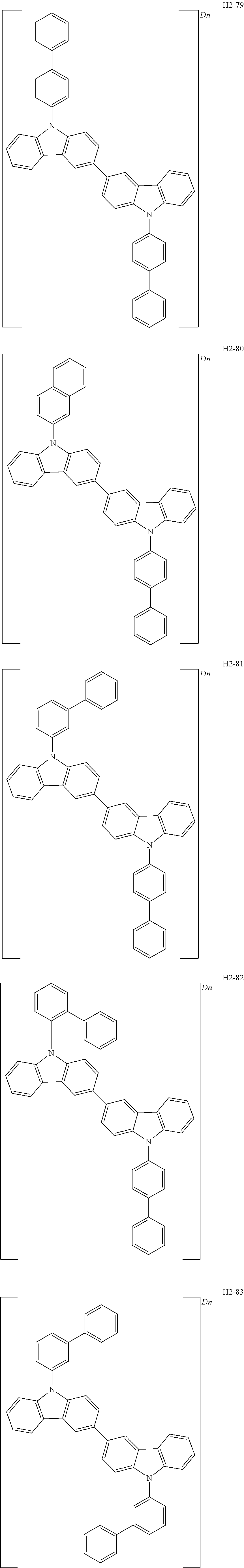

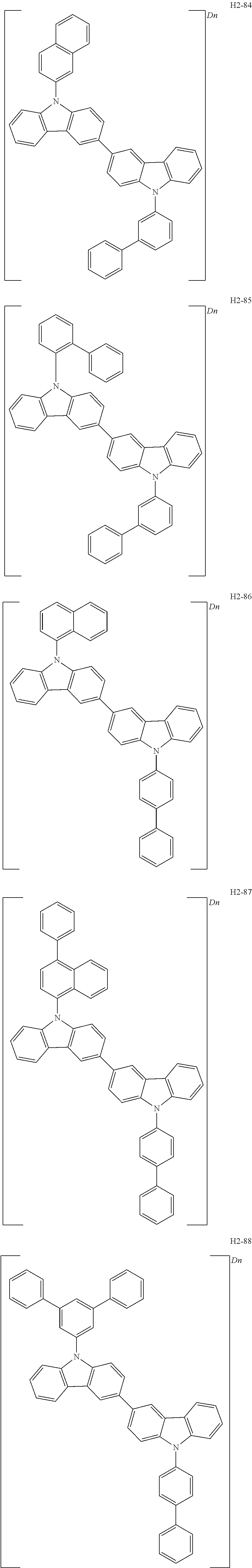

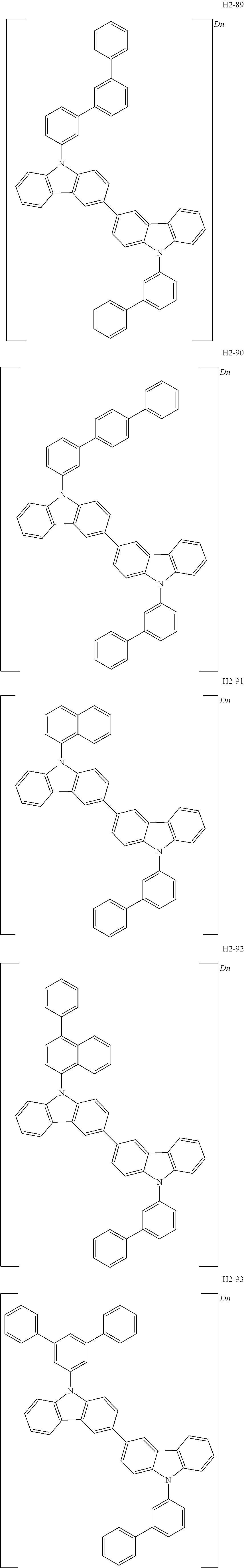

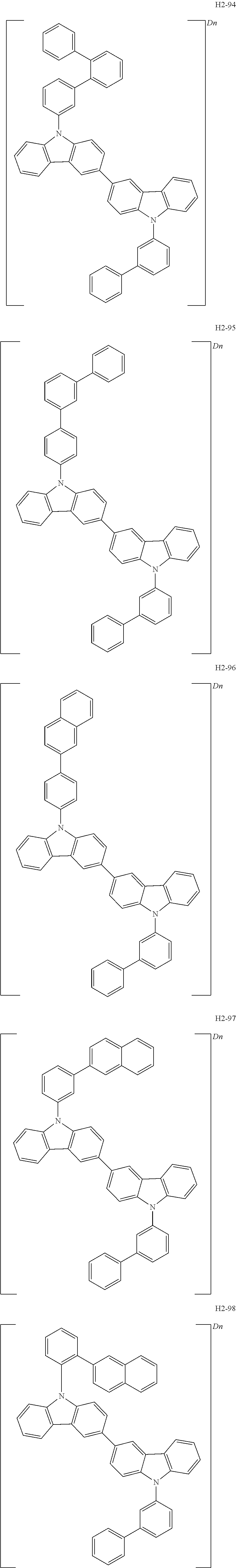

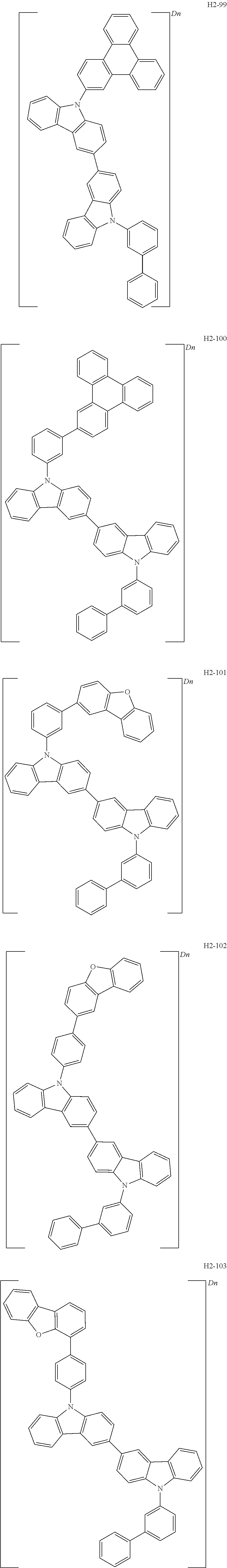

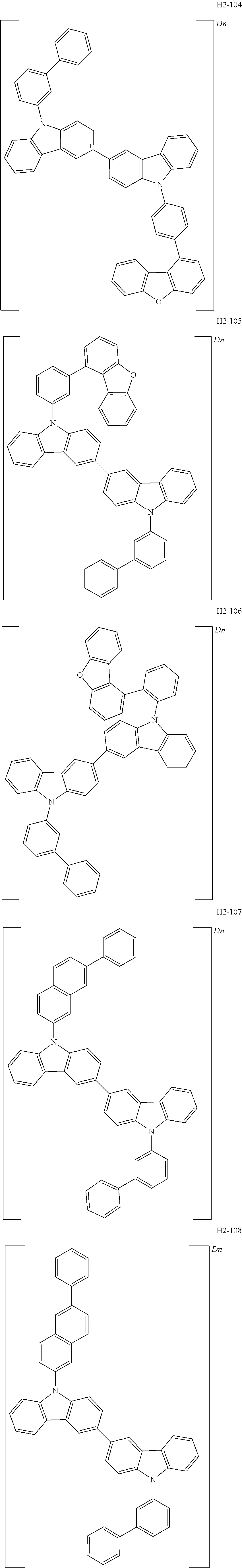

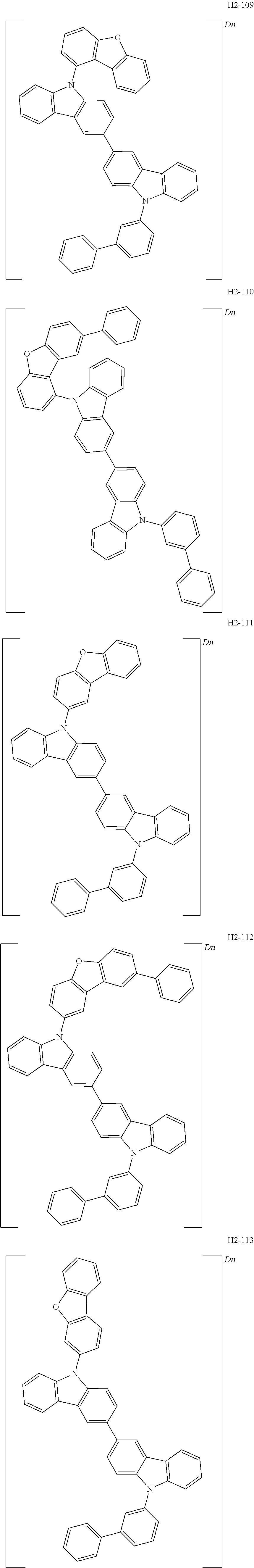

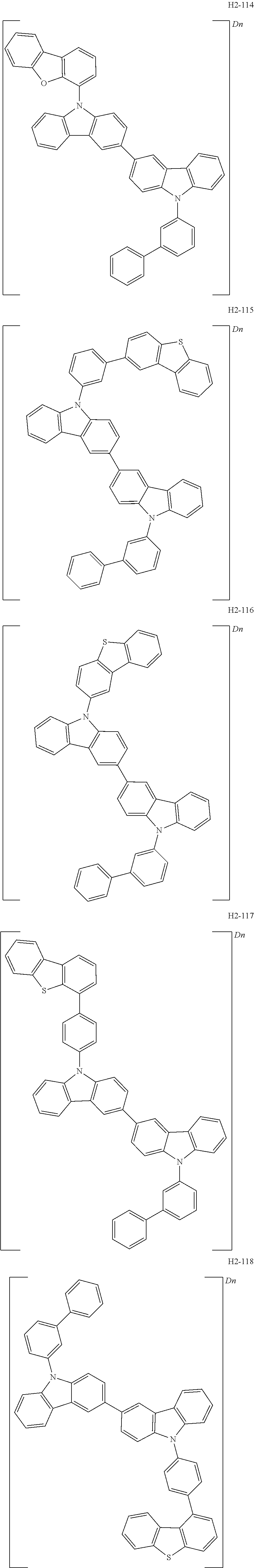

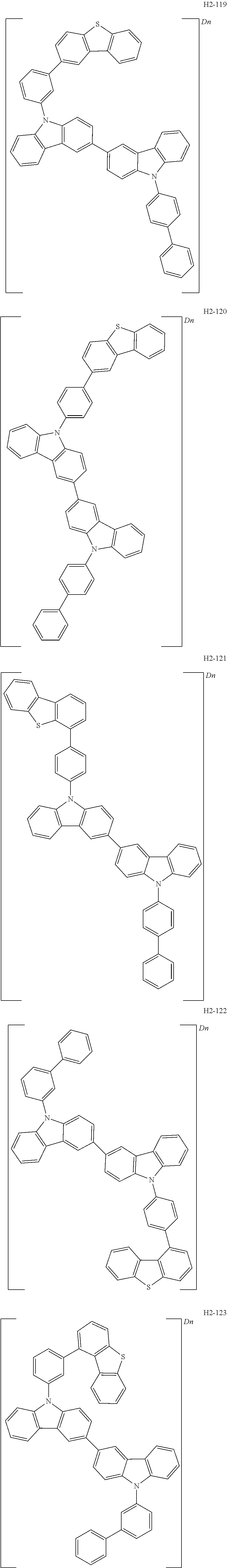

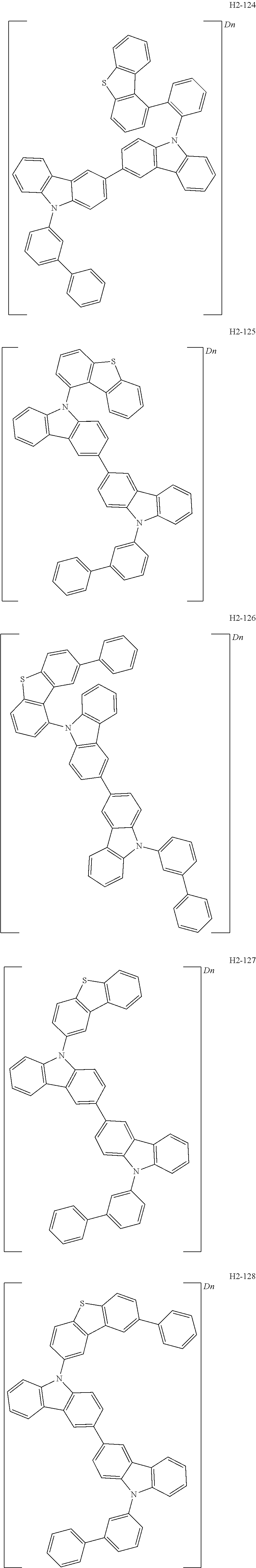

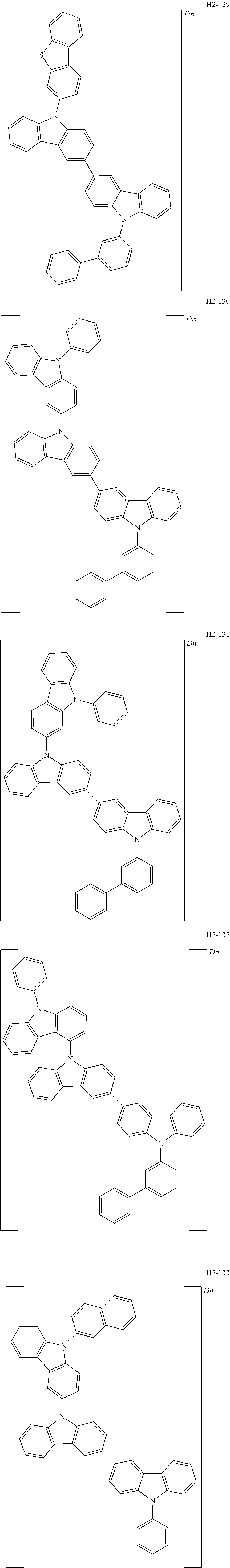

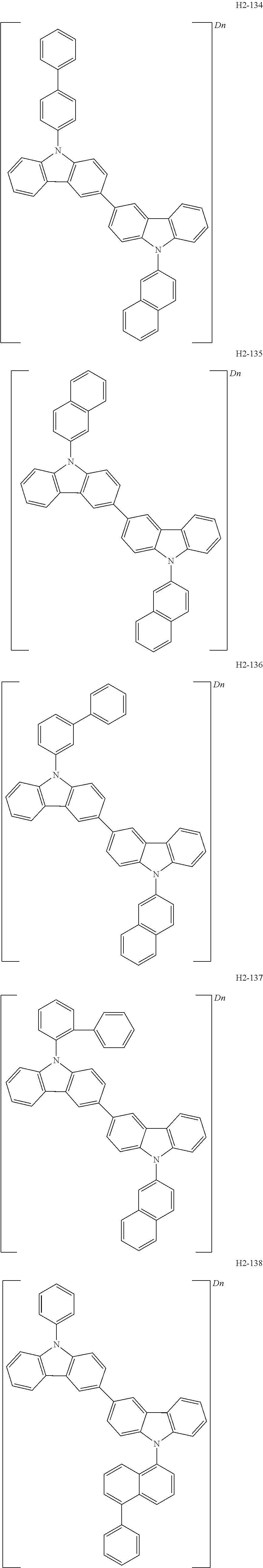

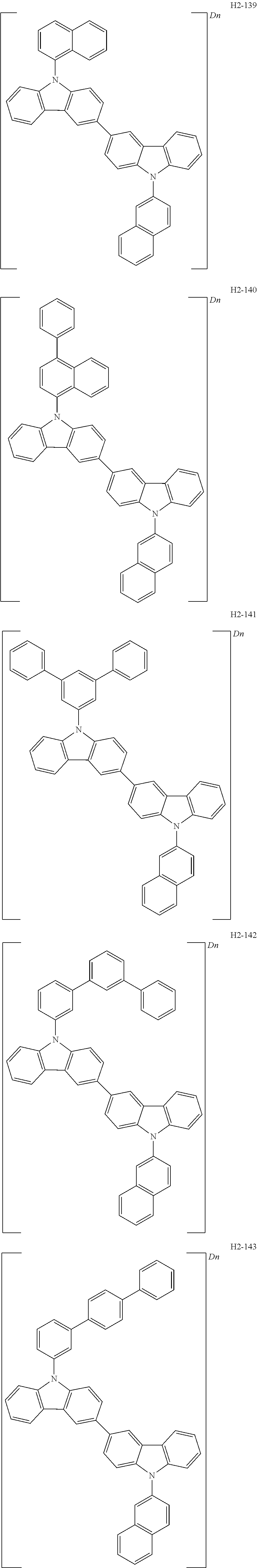

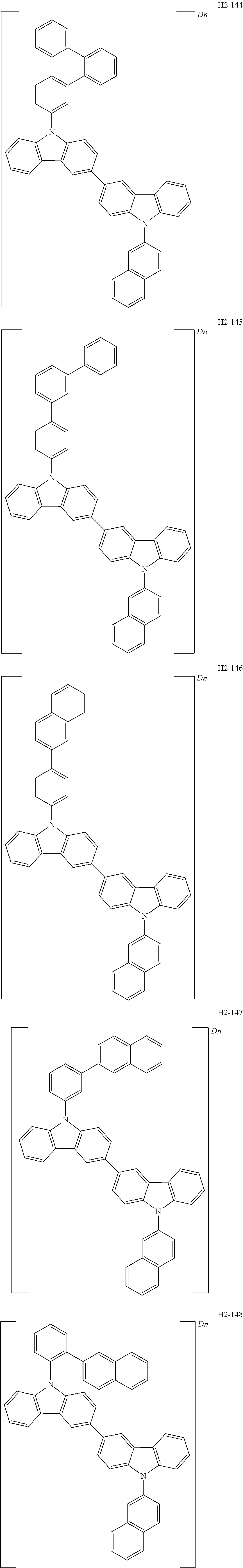

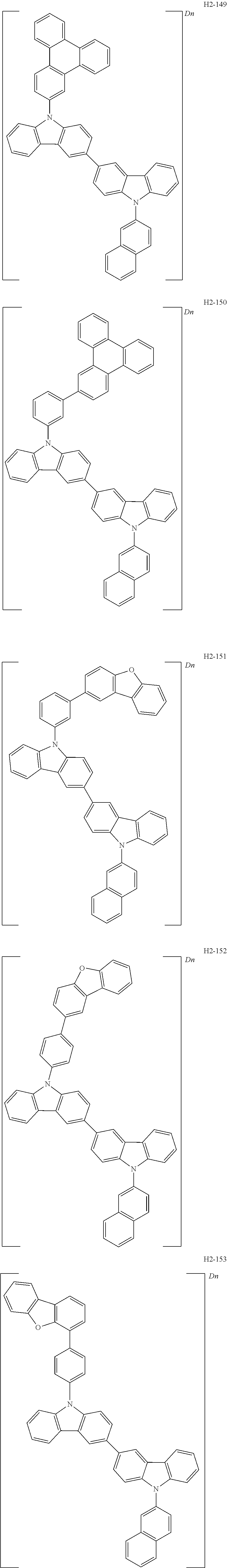

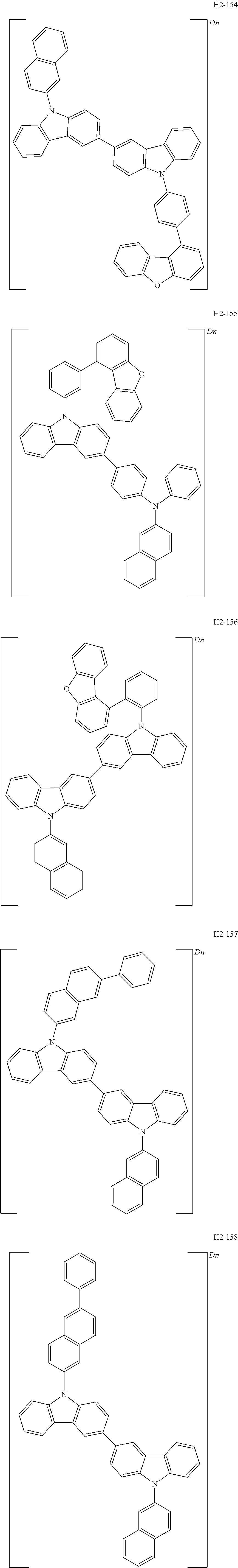

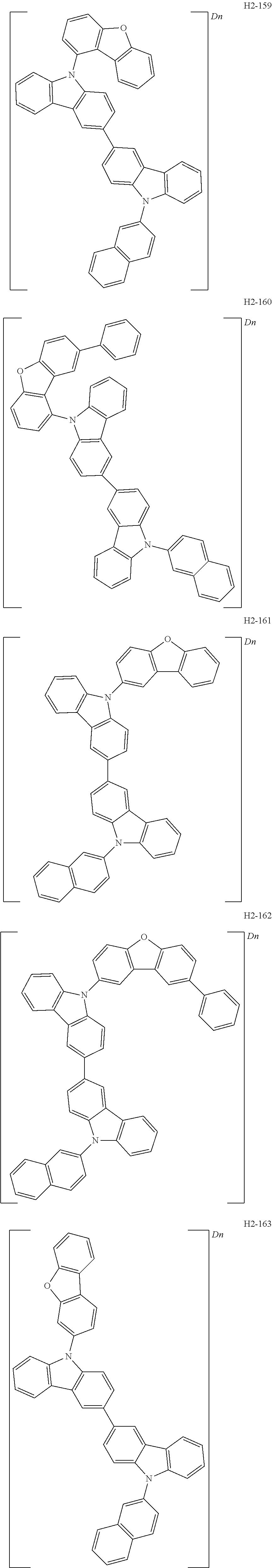

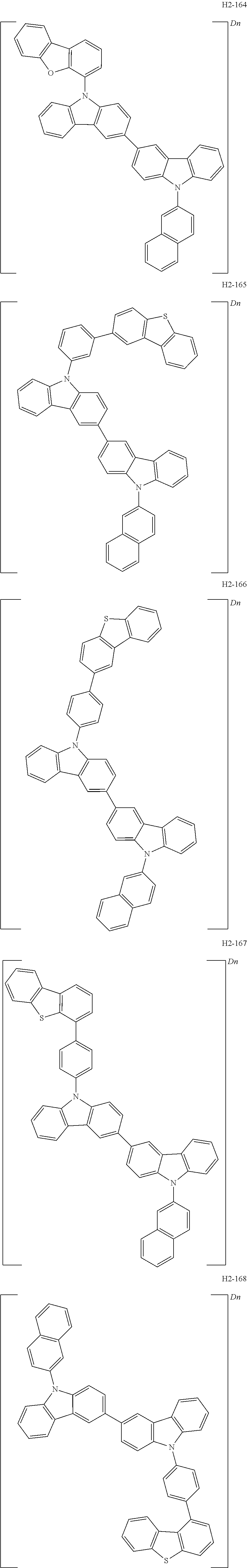

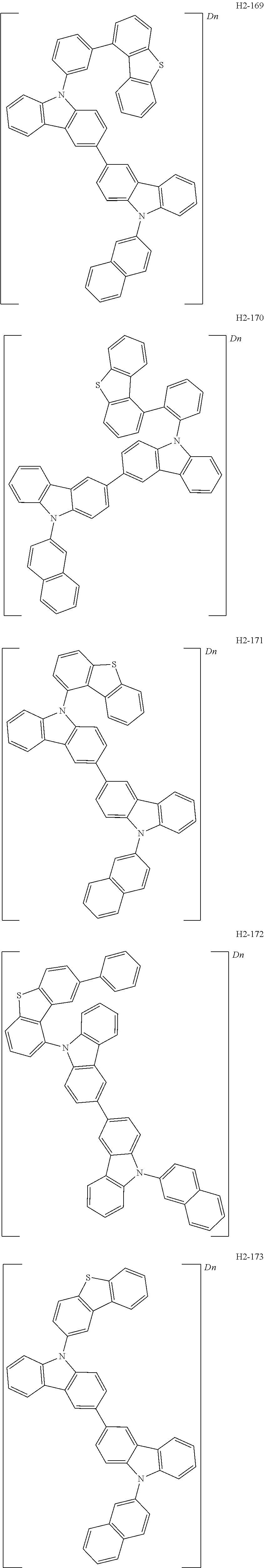

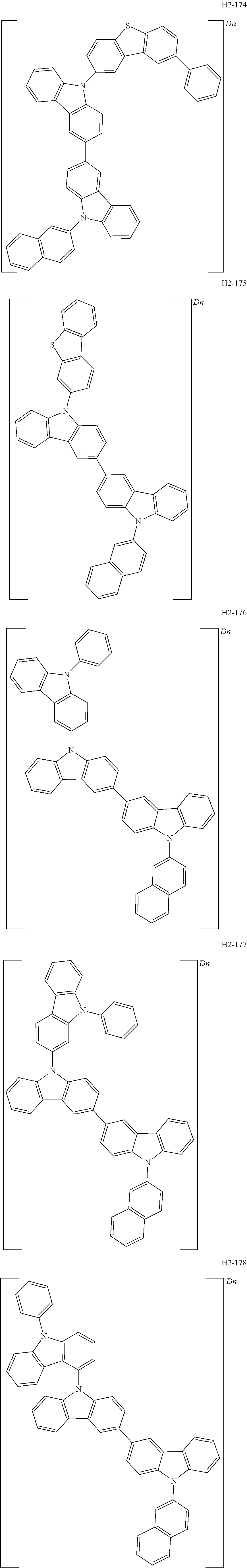

In the formulas above, Dn represents that n number of hydrogens are replaced by deuterium, n represents an integer of 1 or more and is not greater than the number of hydrogen in each compound, n is preferably an integer of 4 or more, and more preferably an integer of 8 or more. When being deuterated to the number of the lower limit or more, the bond dissociation energy related to deuteration may increase to improve stability. When the compound is used in an organic electroluminescent device, the device may exhibit an improved lifetime property.

The combination of at least one of compounds C-1 to C-222 and at least one of compounds H-1 to H-220 and H2-1 to H2-178 may be used in an organic electroluminescent device.

In addition, the present disclosure provides an organic electroluminescent compound represented by the following formula 4:

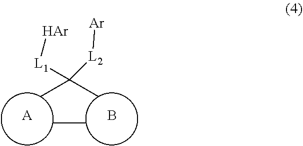

in formula 4,

ring A and ring B, each independently, represent a substituted or unsubstituted benzene ring, a substituted or unsubstituted naphthalene ring, or a substituted or unsubstituted phenanthrene ring;

L₁ represents a single bond;

L₂ represents a single bond, or a substituted or unsubstituted (C6-C30)arylene;

Ar represents a substituted or unsubstituted (C6-C30)aryl, a substituted or unsubstituted dibenzofuranyl, a substituted or unsubstituted dibenzothiophenyl, or a substituted or unsubstituted carbazolyl;

HAr represents a substituted or unsubstituted (3- to 20-membered)heteroaryl containing a nitrogen atom(s);

with the proviso that HAr is not a heteroaryl substituted with a substituent

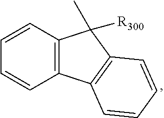

in which R₃₀₀ represents a (C1-C30)alkyl, or a (C6-C30)aryl, and * represents a bonding site of the substituent; and

the compound represented b formula 4 is not the following compounds:

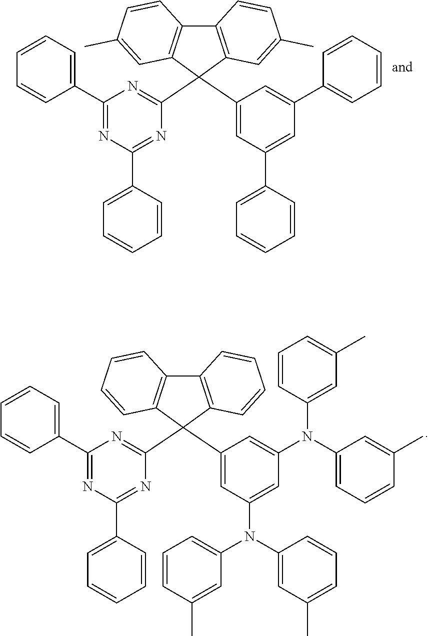 [SMILES: Cc1ccc2c(c1)C(c1cc(-c3ccccc3)cc(-c3ccccc3)c1)(c1nc(-c3ccccc3)nc(-c3ccccc3)n1)c1cc(C)ccc1-2 ; Cc1cccc(N(c2cccc(C)c2)c2cc(N(c3cccc(C)c3)c3cccc(C)c3)cc(C3(c4nc(-c5ccccc5)nc(-c5ccccc5)n4)c4ccccc4-c4ccccc43)c2)c1]

According to one embodiment of the present disclosure, in formula 4, ring A and ring B, each independently, represent an unsubstituted benzene ring, an unsubstituted naphthalene ring, or an unsubstituted phenanthrene ring.

According to one embodiment of the present disclosure, in formula 4, L₁ and L₂ represent a single bond.

According to one embodiment of the present disclosure, in formula 4, Ar represents an unsubstituted (C6-C18)aryl, an unsubstituted dibenzofuranyl, an unsubstituted dibenzothiophenyl, or a carbazolyl substituted with a phenyl(s), in which the (C6-C18)aryl may be, for example, a phenyl, a naphthyl, or biphenyl, etc.

According to one embodiment of the present disclosure, in formula 4, HAr represents a substituted (5- to 10-membered)heteroaryl containing a nitrogen atom(s). For example, HAr may be a substituted triazinyl, in which the substituent(s) of the substituted triazinyl may be at least one, preferably two, selected from the group consisting of a phenyl unsubstituted or substituted with a dibenzofuranyl(s); a naphthyl unsubstituted or substituted with a dibenzofuranyl(s); a biphenyl unsubstituted or substituted with a dibenzofuranyl(s); a phenanthrenyl; a chrysenyl; a dibenzofuranyl; a dibenzothiophenyl; a phenanthrooxazolyl substituted with a phenyl(s); a carbazolyl substituted with a phenyl(s); a dibenzocarbazolyl; and a (23-membered)heteroaryl containing a nitrogen atom(s).

The compound represented by formula 4 may be at least one selected from the group consisting of compounds C-1 to C-145, C-156 to C-161, and C-195 to C-222 above, but is not limited thereto.

The compound represented by formula 1 or 4 according to the present disclosure may be produced by a synthetic method known to one skilled in the art, and for example, by referring to the following reaction schemes, but is not limited thereto. The compound represented by formula 2 according to the present disclosure may be produced by a synthetic method known to one skilled in the art, in particular the synthetic methods disclosed in many patent documents, for example, by referring to Korean Patent Application Laying-Open No, 2017-0022865 (published on Mar. 2, 2017), but is not limited thereto.

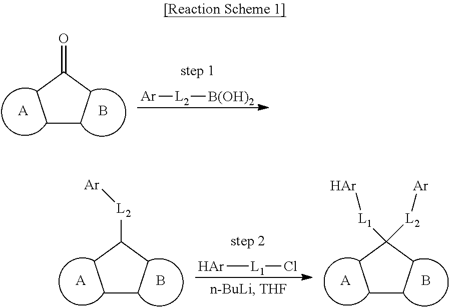

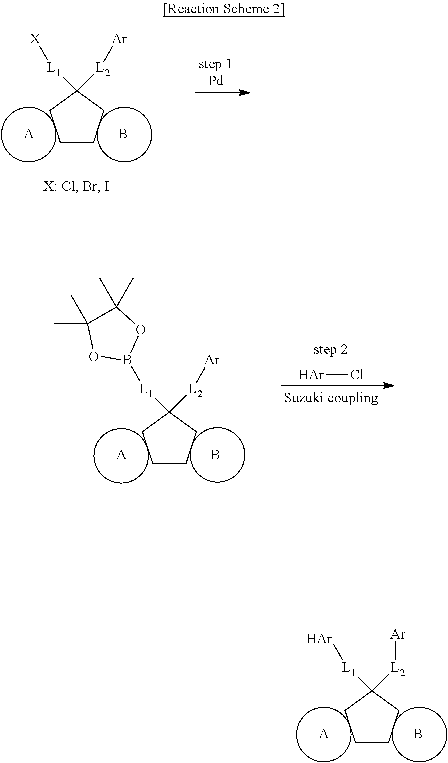

In reaction schemes 1 and 2, ring A, ring B, L₁, L₂, Ar, and HAr are as defined in formula 1 or 4.

Although illustrative synthesis examples of the compound represented by formula 1 or 4 of the present disclosure are described above, one skilled in the art will be able to readily understand that all of them are based on a Buchwald-Hartwig cross-coupling reaction, an N-arylation reaction, a H-mont-mediated etherification reaction, a Miyaura borylation reaction, a Suzuki cross-coupling reaction, an Intramolecular acid-induced cyclization reaction, a Pd(II)-catalyzed oxidative cyclization reaction, a Grignard reaction, a Heck reaction, a Cyclic Dehydration reaction, an SN₁ substitution reaction, an SN₂ substitution reaction, and a Phosphine-mediated reductive cyclization reaction, etc., and the reactions above proceed even when substituents which are defined in formula 1 or 4 above, but are not specified in the specific synthesis examples, are bonded.

The present disclosure provides an organic electroluminescent device comprising an anode, a cathode, and at least one light-emitting layer between the anode and cathode in which the light-emitting layer comprises a plurality of host materials according to the present disclosure. The first host material and the second host material may be comprised in one light-emitting layer, or may be respectively comprised in different light-emitting layers. The ratio of the compound represented by formula 1 and the compound represented by formula 2 in the plurality of host materials is about 1:99 to about 99:1, preferably about 10:90 to about 90:10, more preferably about 30:70 to about 70:30. In addition, the compound represented by formula 1 and the compound represented by formula 2 may be combined by mixing them in a shaker, by dissolving them in a glass tube by heat, or by dissolving them in a solvent, etc.

According to one embodiment of the present disclosure, the doping concentration of the dopant compound with respect to the host compound in the light-emitting layer may be less than 20 wt %. The dopant comprised in the organic electroluminescent device of the present disclosure may be at least one phosphorescent or fluorescent dopant, and is preferably a phosphorescent dopant. The phosphorescent dopant material applied to the organic electroluminescent device of the present disclosure is not particularly limited, but may be preferably selected from the metallated complex compounds of iridium (Ir), osmium (Os), copper (Cu), and platinum (Pt), more preferably selected from ortho-metallated complex compounds of iridium (Ir), osmium (Os), copper (Cu), and platinum (Pt), and even more preferably ortho-metallated iridium complex compounds.

The dopant comprised in the organic electroluminescent device of the present disclosure may comprise a compound represented by the following formula 101, but is not limited thereto.

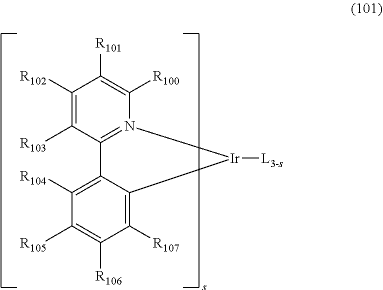

In formula 101,

L is selected from the following structures 1 to 3:

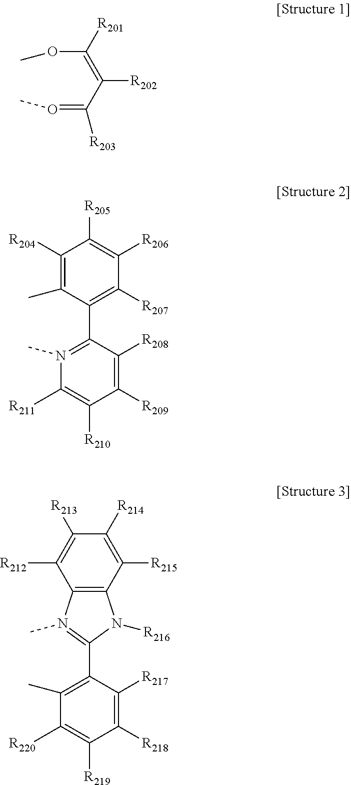

R₁₀₀ to R₁₀₃, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent to form a ring(s), e.g., a substituted or unsubstituted, quinoline, benzofuropyridine, benzothienopyridine, indenopyridine, benzofuroquinoline, benzothienoquinoline, or indenoquinoline, together with pyridine;

R₁₀₄ to R₁₀₇, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent to form a ring(s), e.g., a substituted or unsubstituted, naphthalene, fluorene, dibenzothiophene, dibenzofuran, indenopyridine, benzofuropyridine, or benzothienopyridine, together with benzene;

R₂₀₁ to R₂₂₀, each independently, represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30)aryl; or may be linked to an adjacent substituent to form a substituted or unsubstituted ring(s); and

s represents an integer of 1 to 3.

The specific examples of the dopant compound are as follows, but are not limited thereto.

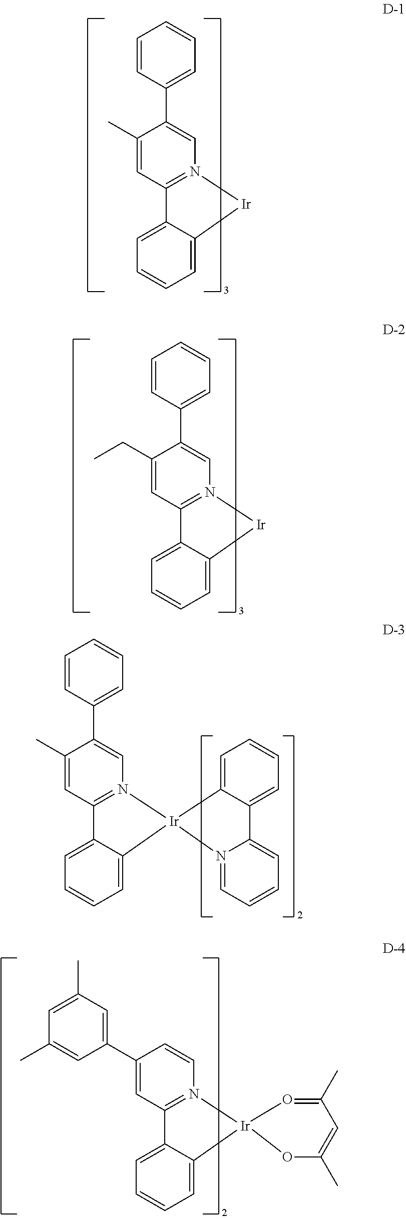

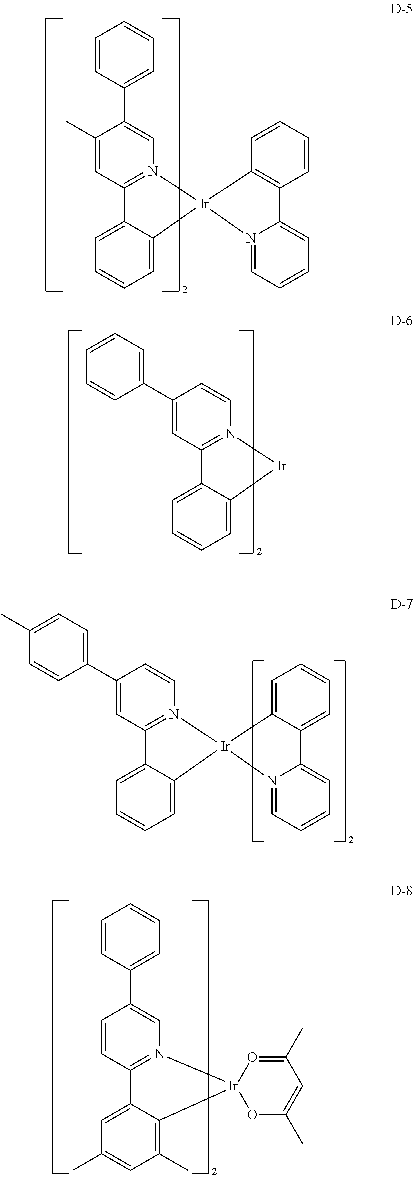

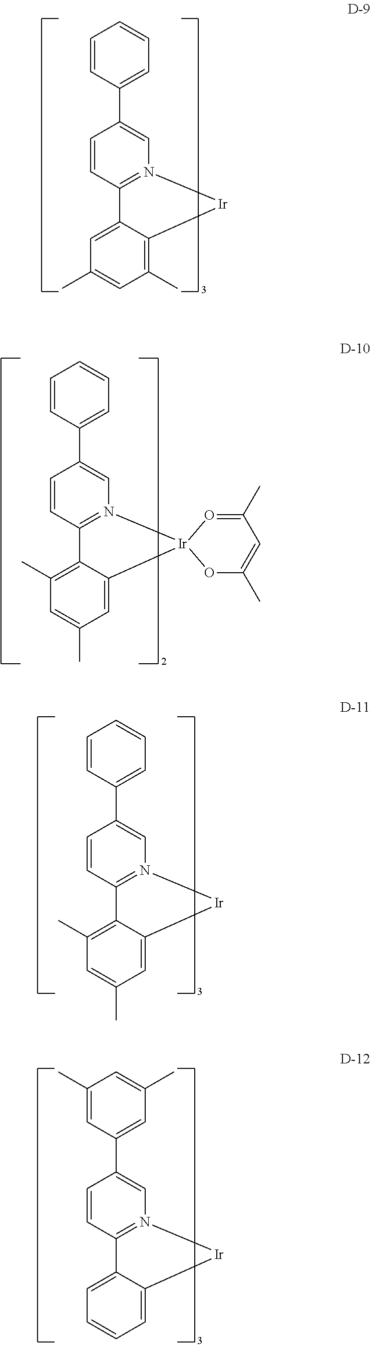

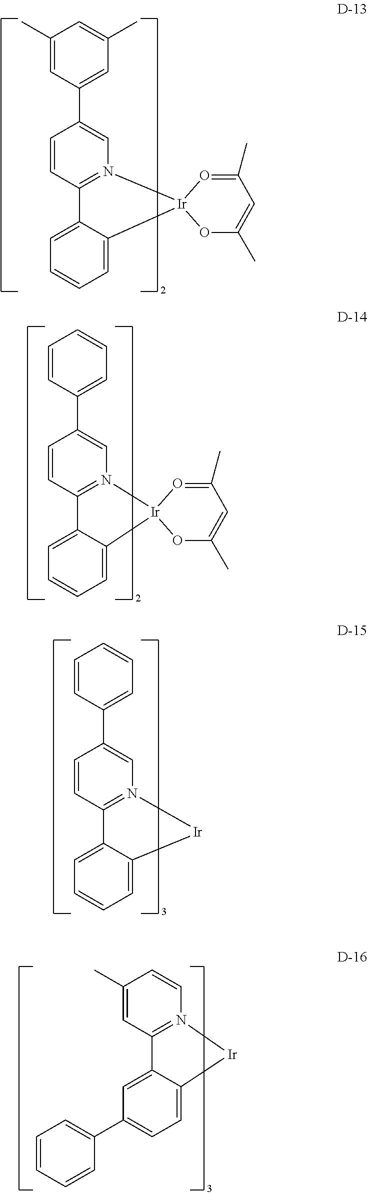

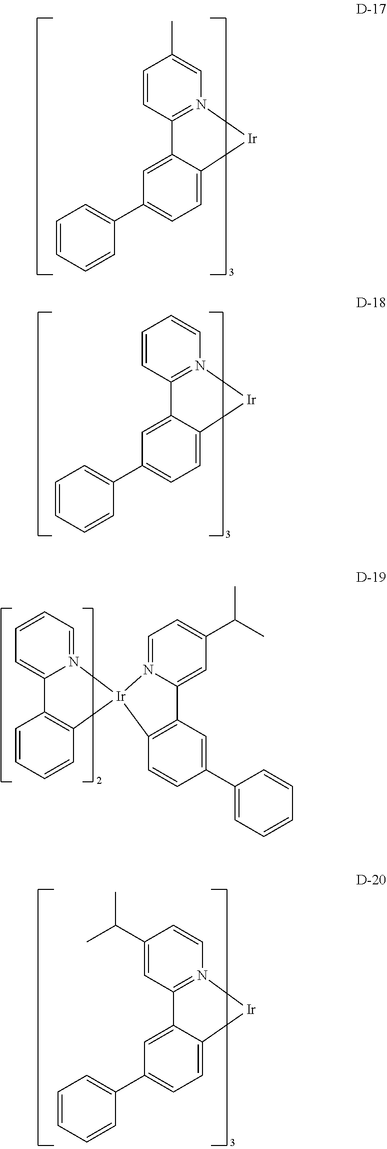

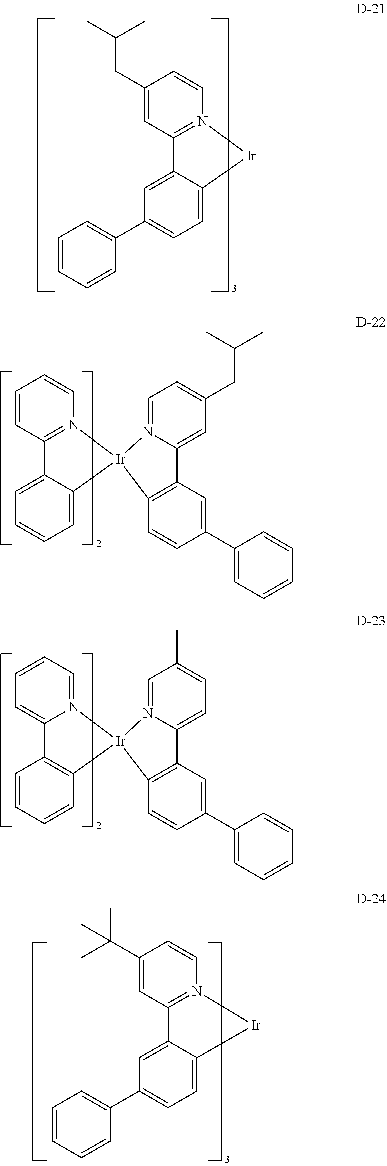

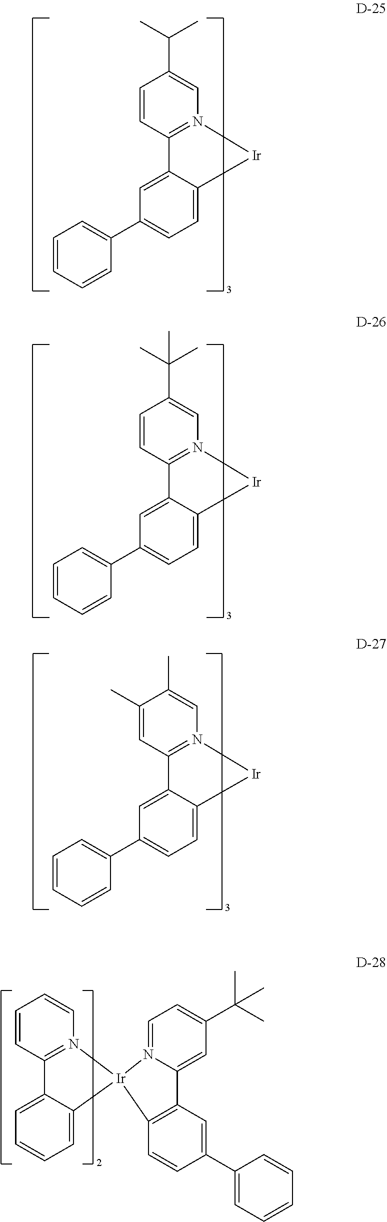

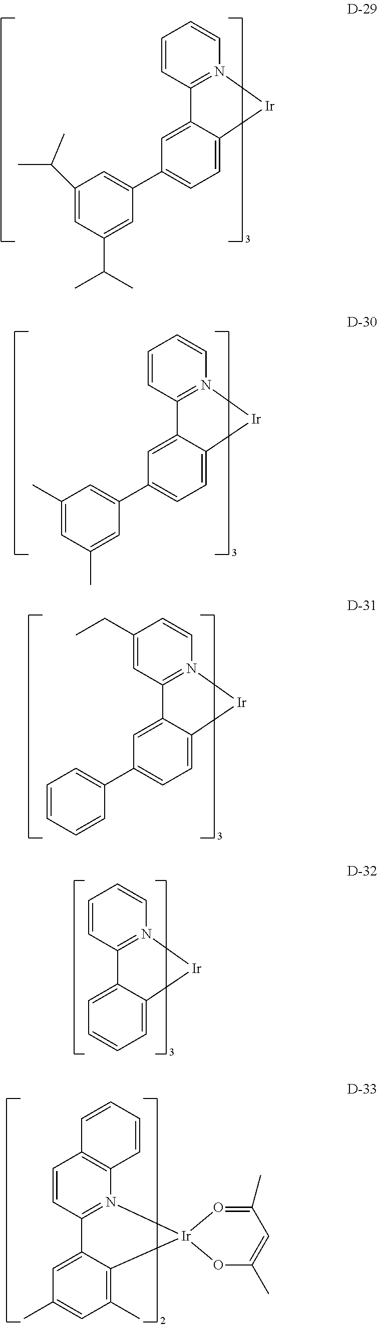

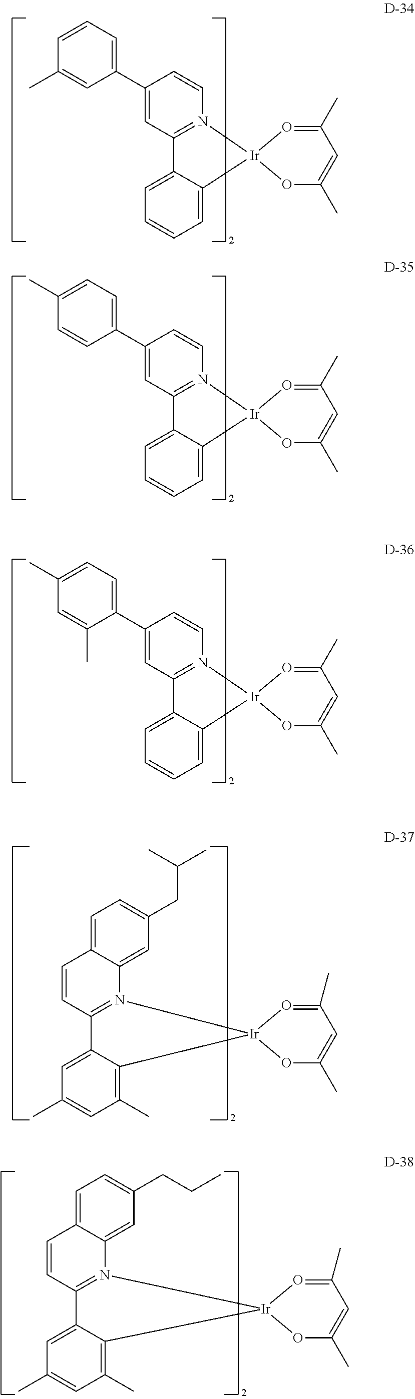

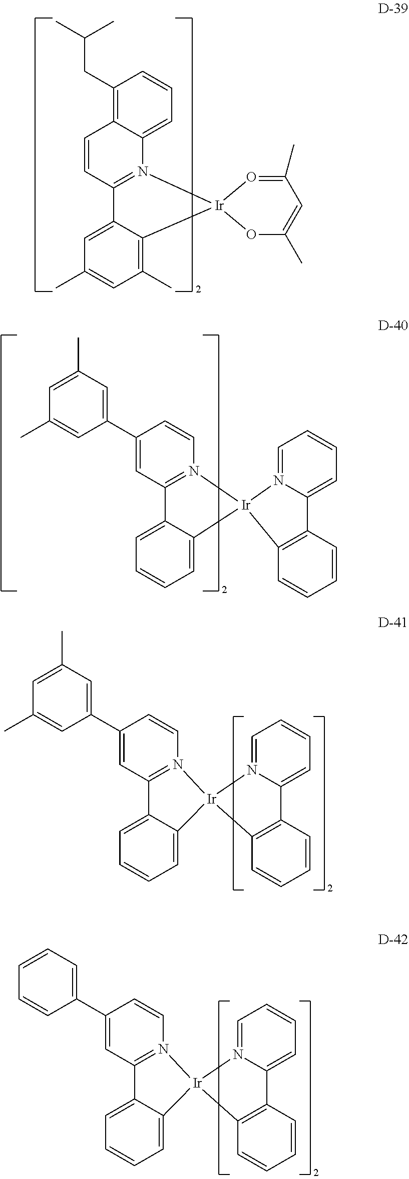

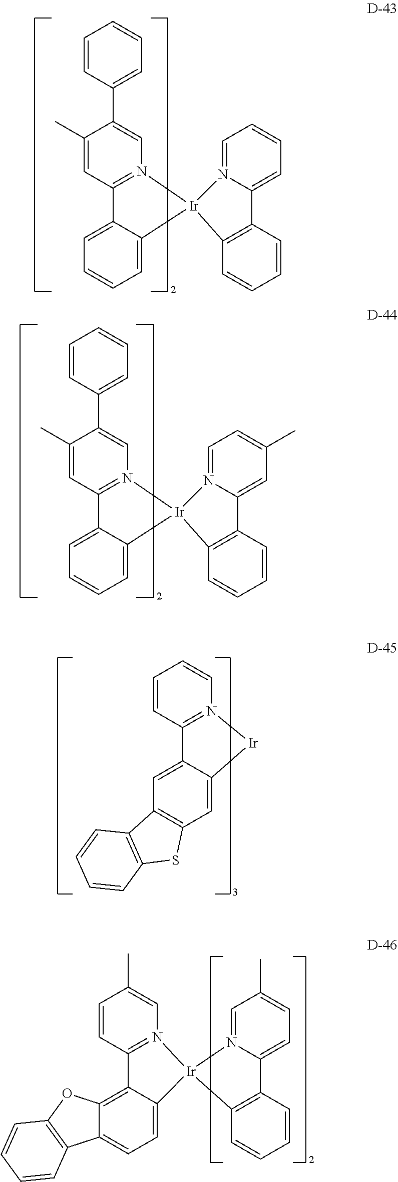

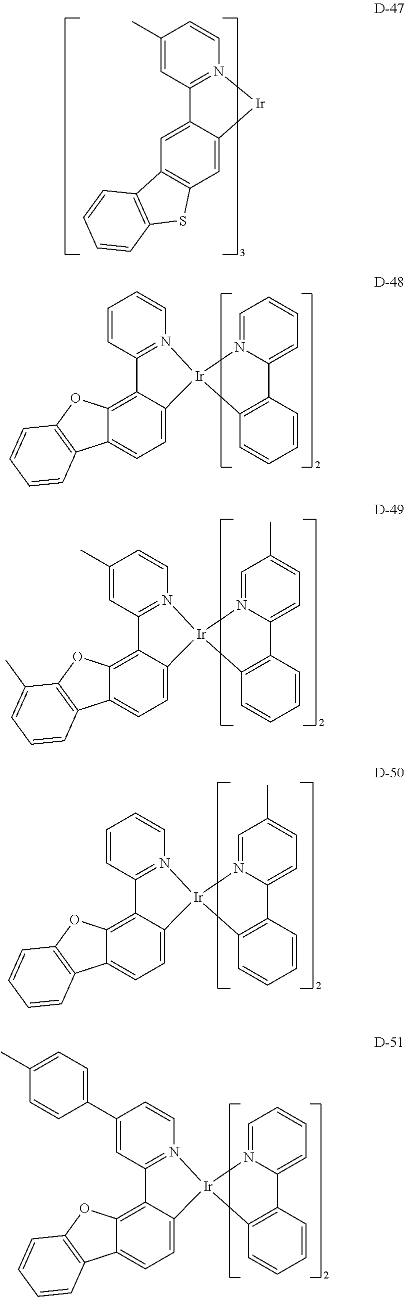

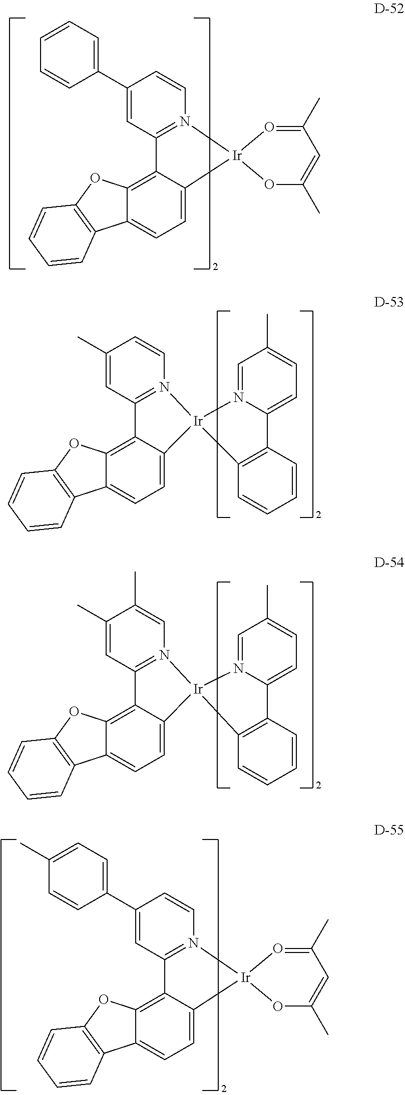

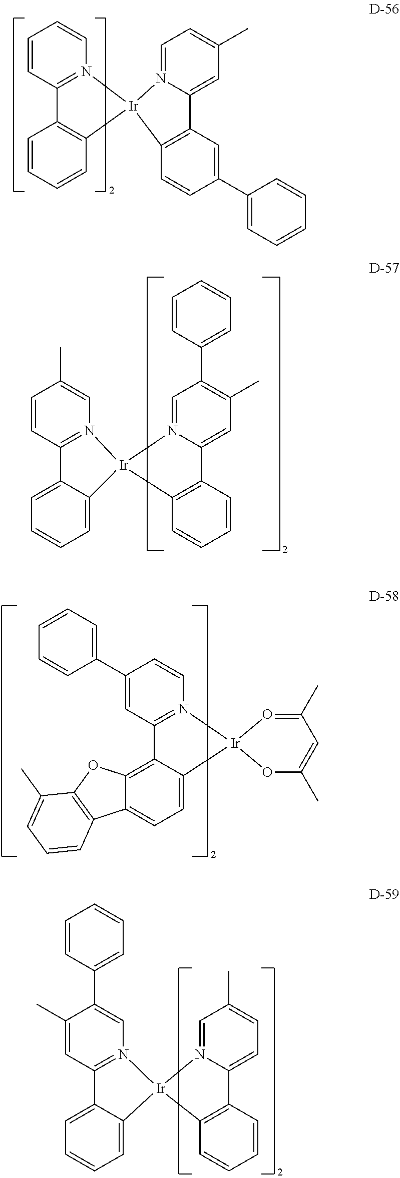

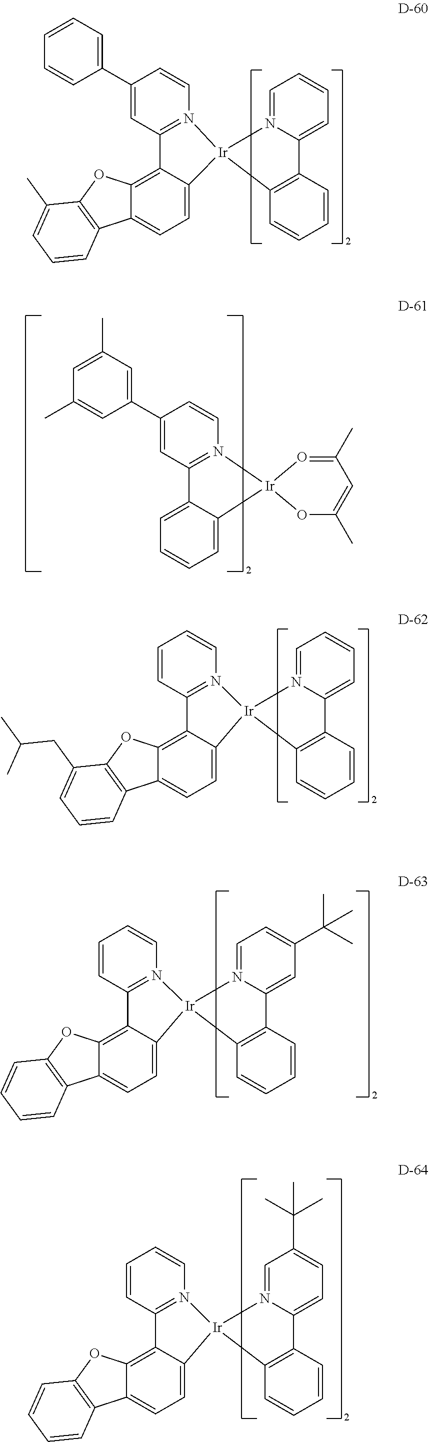

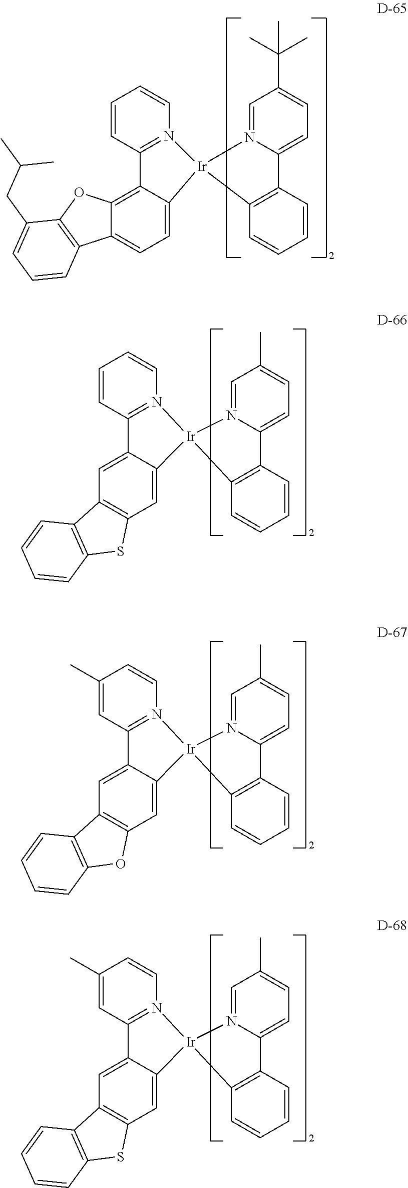

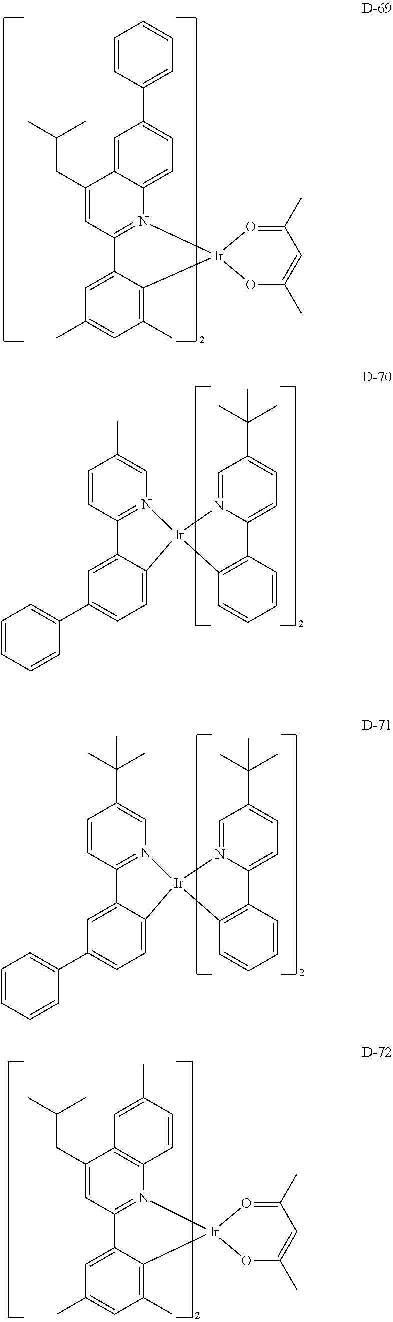

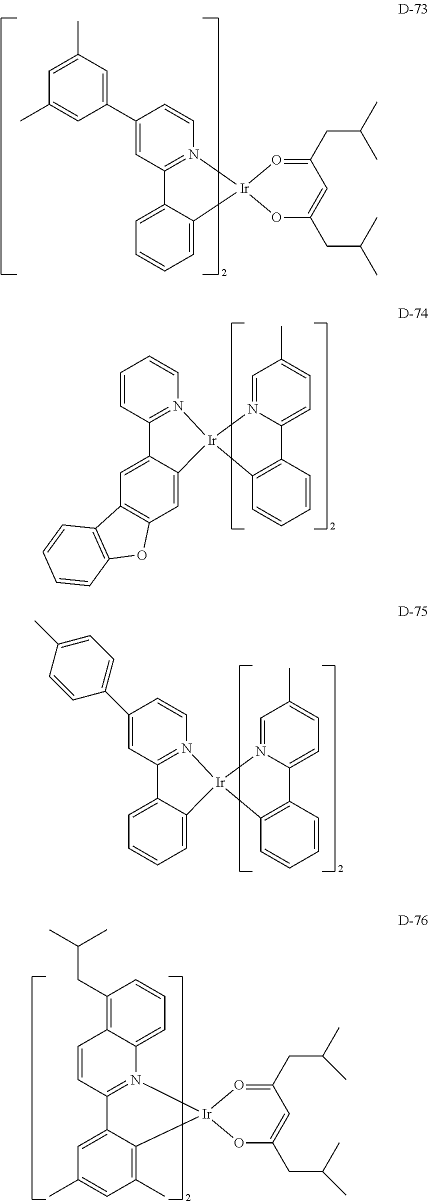

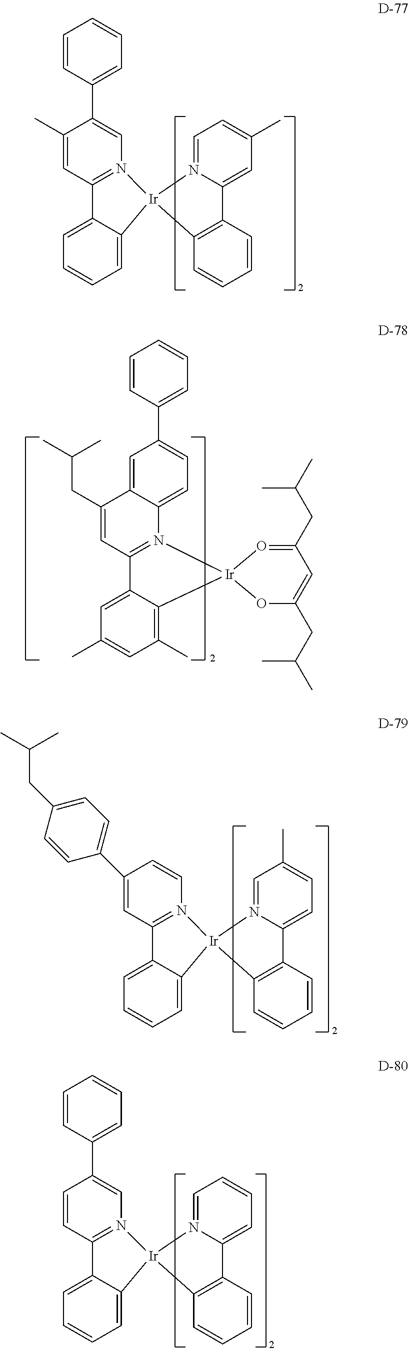

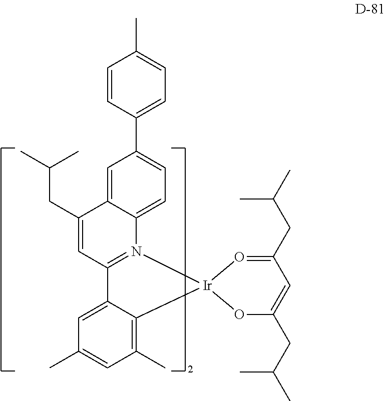

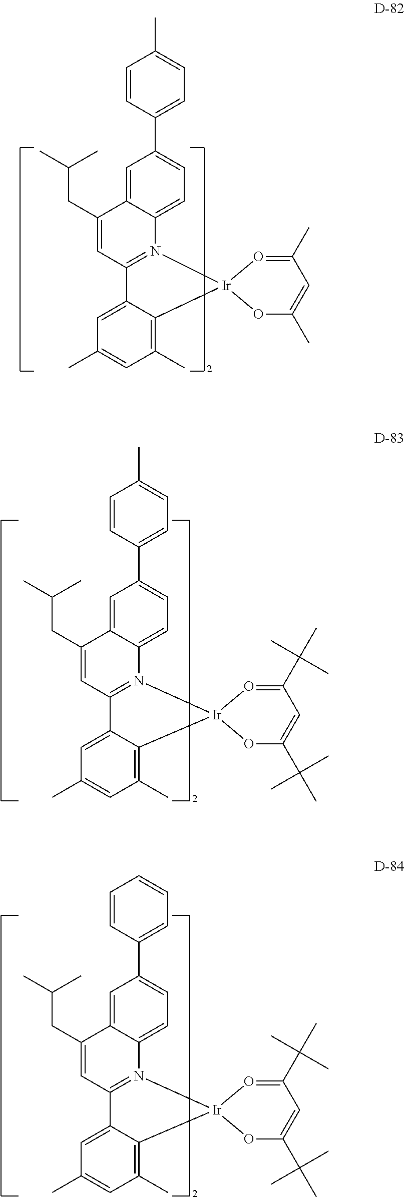

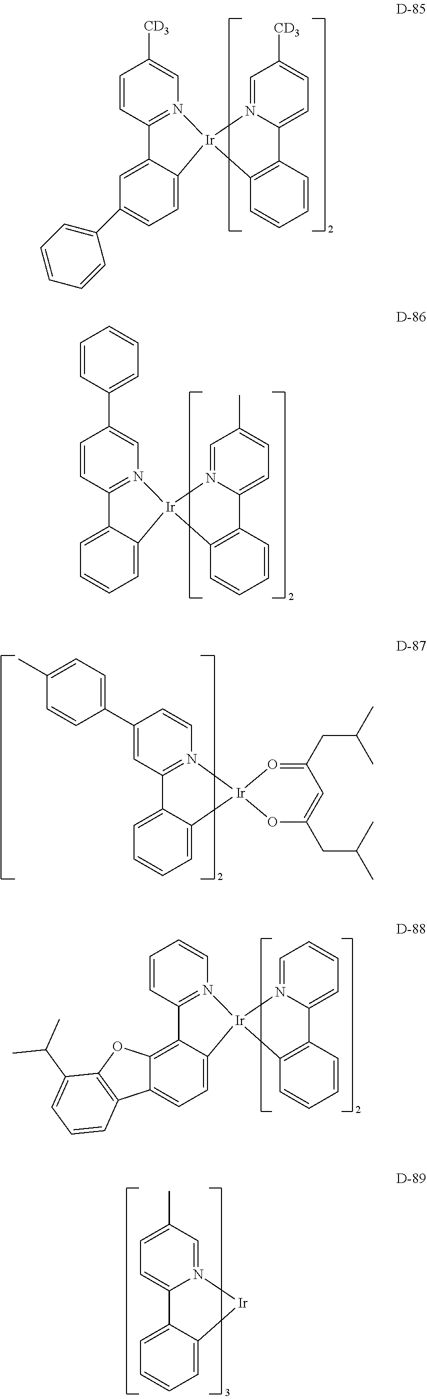

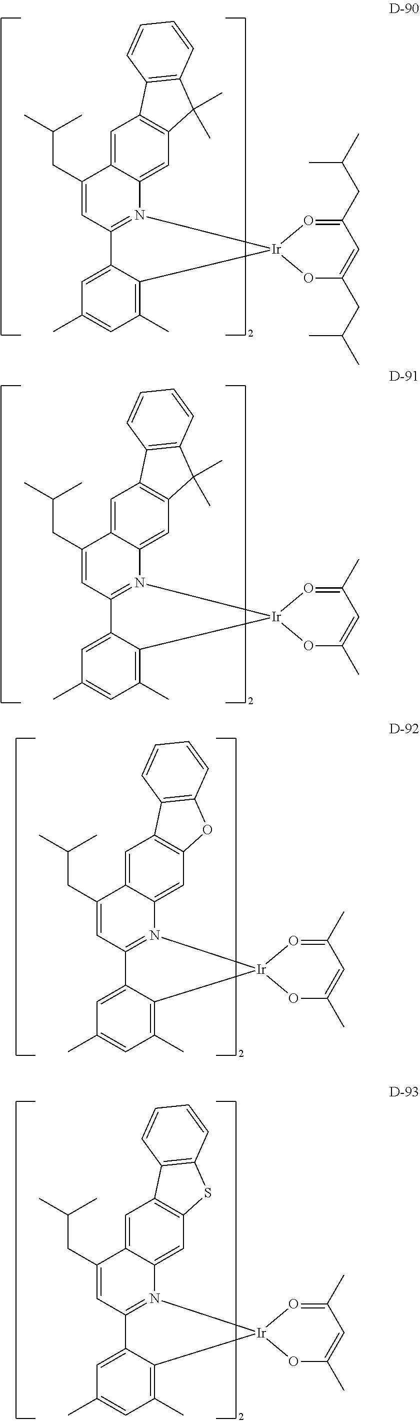

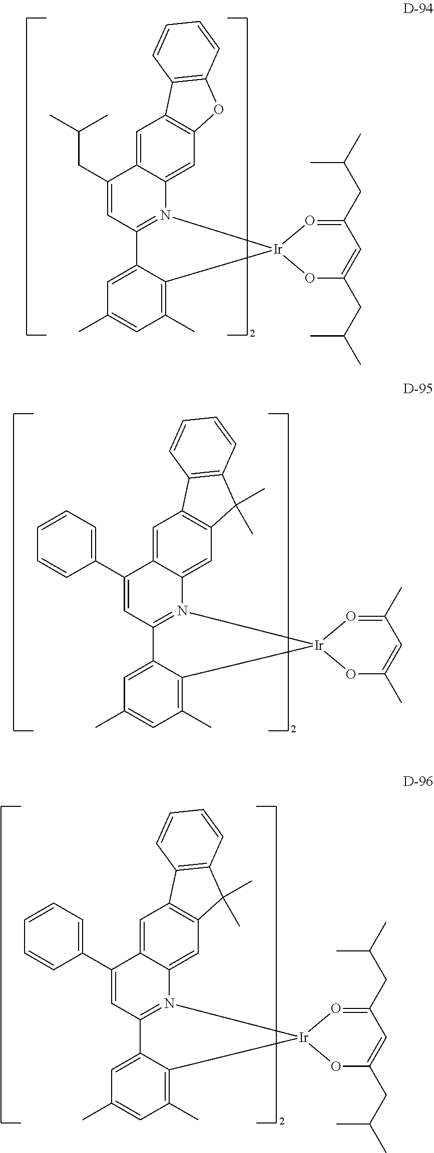

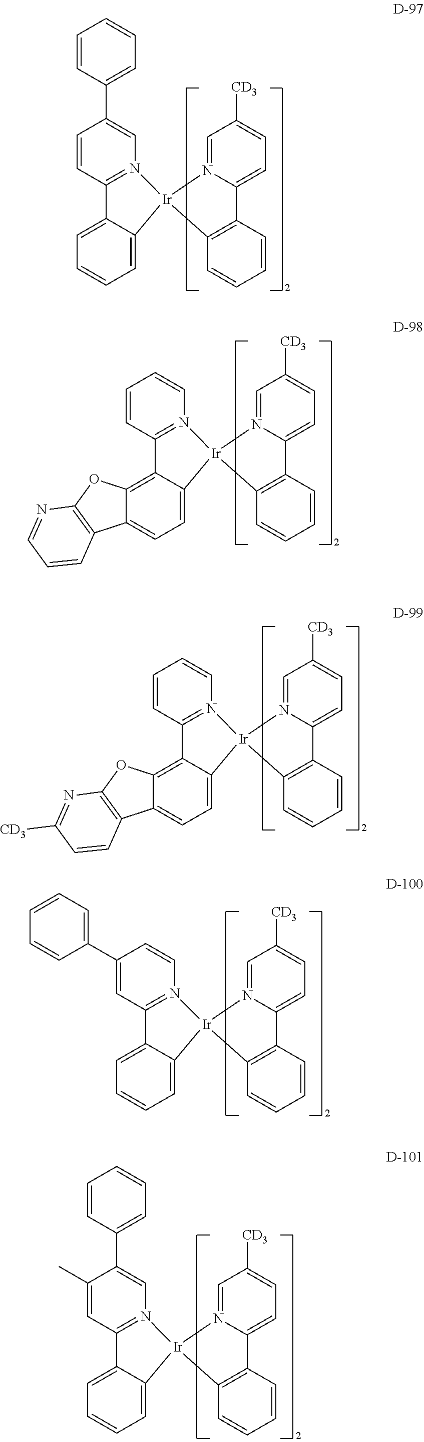

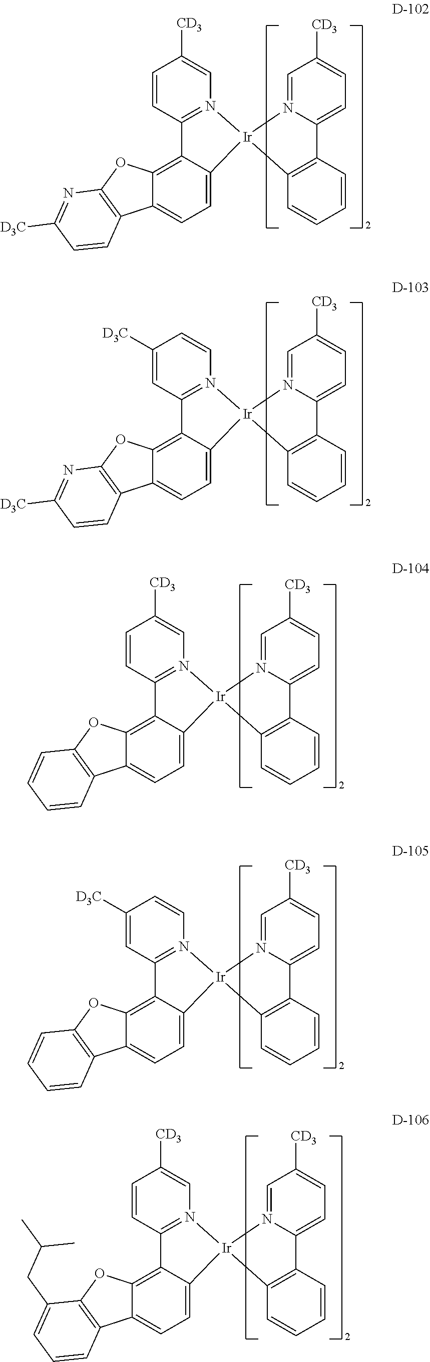

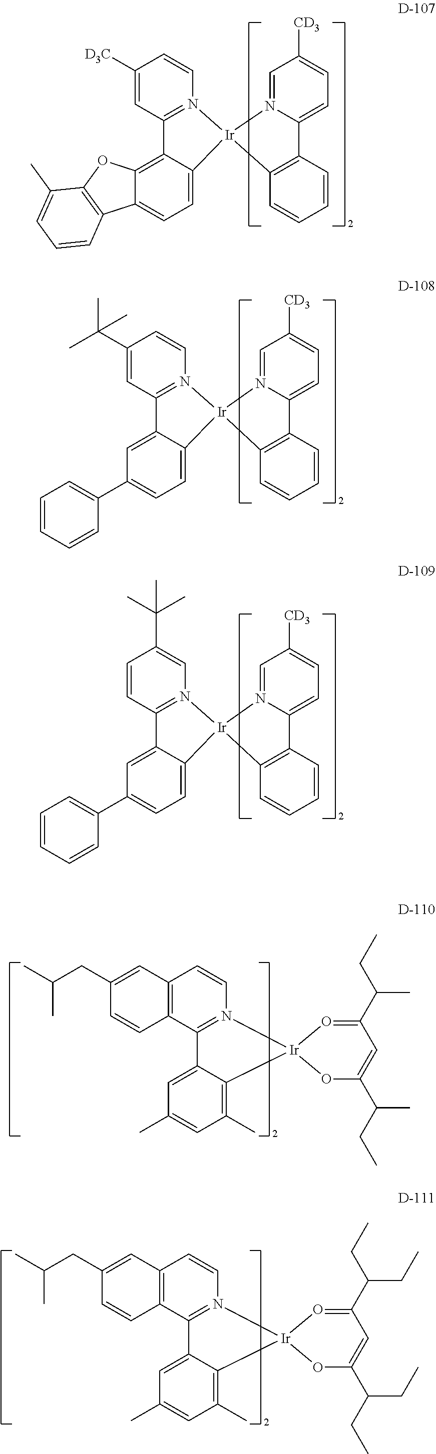

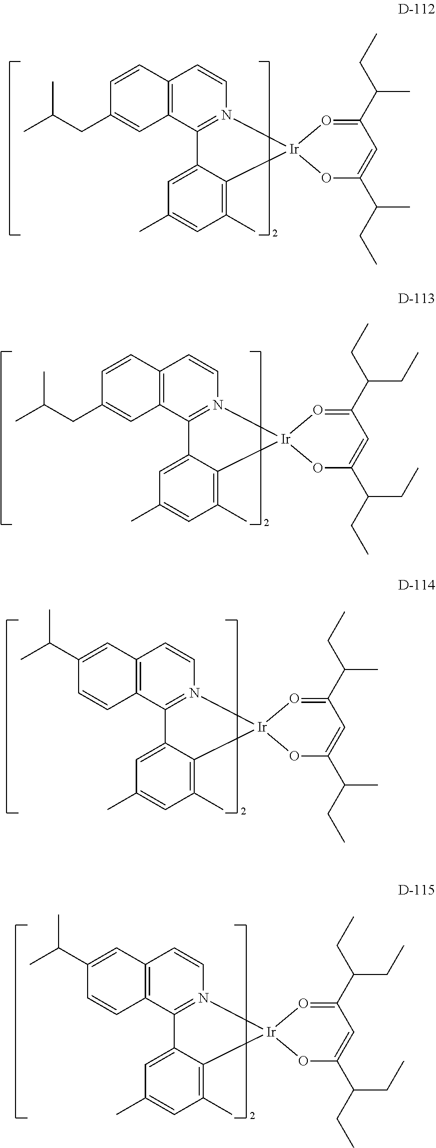

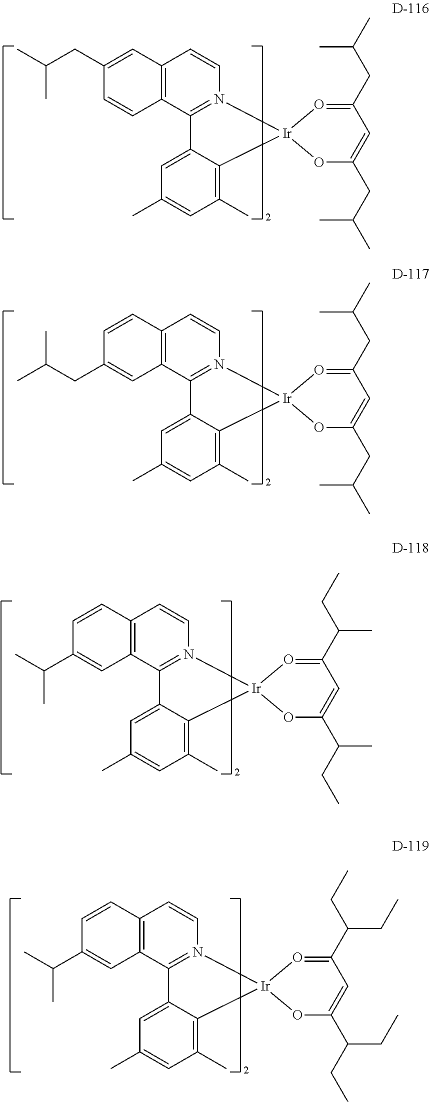

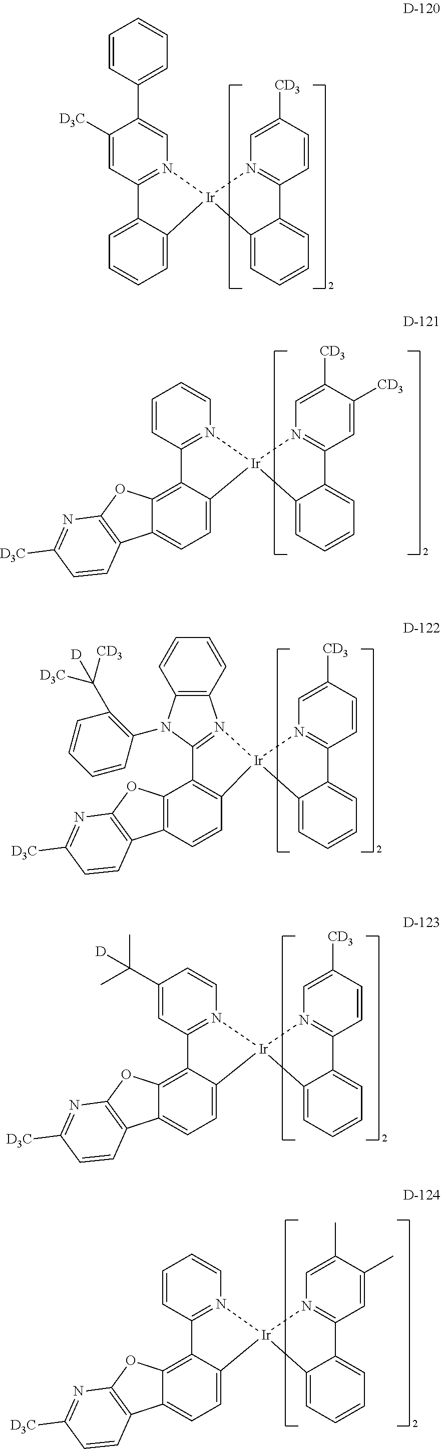

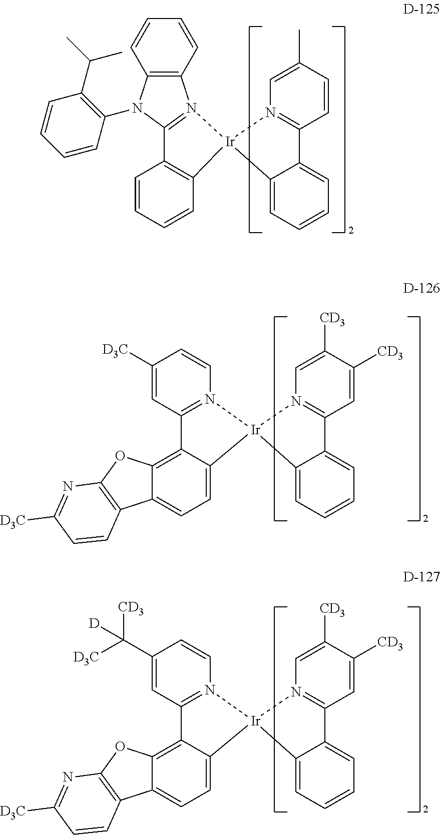

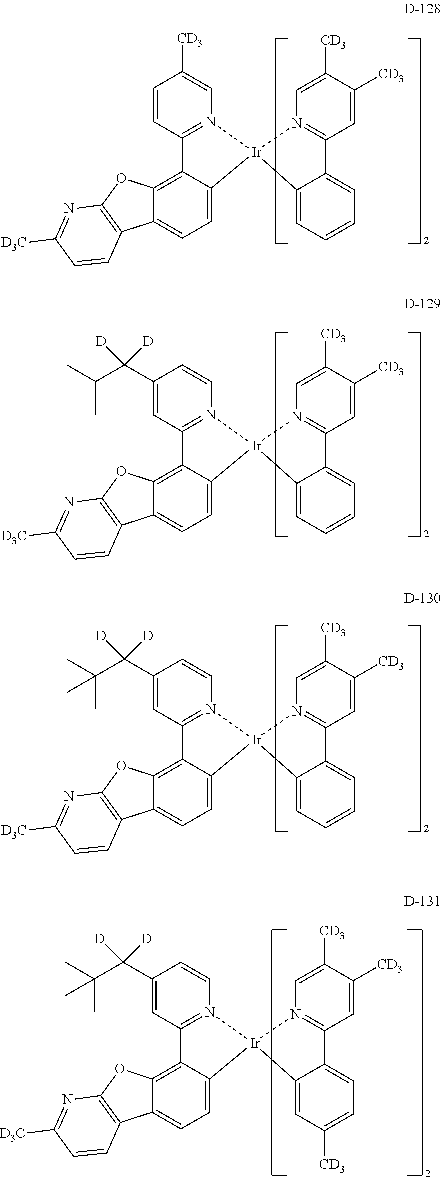

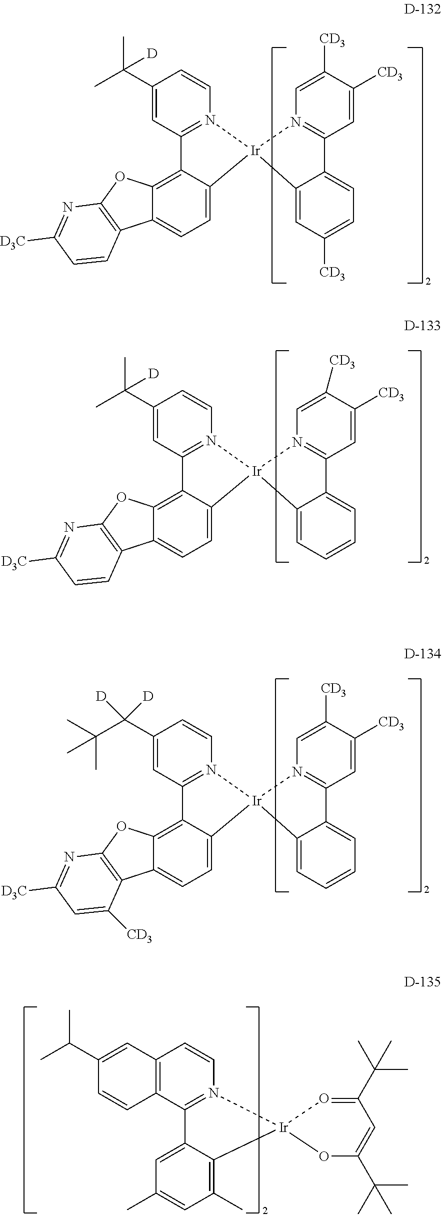

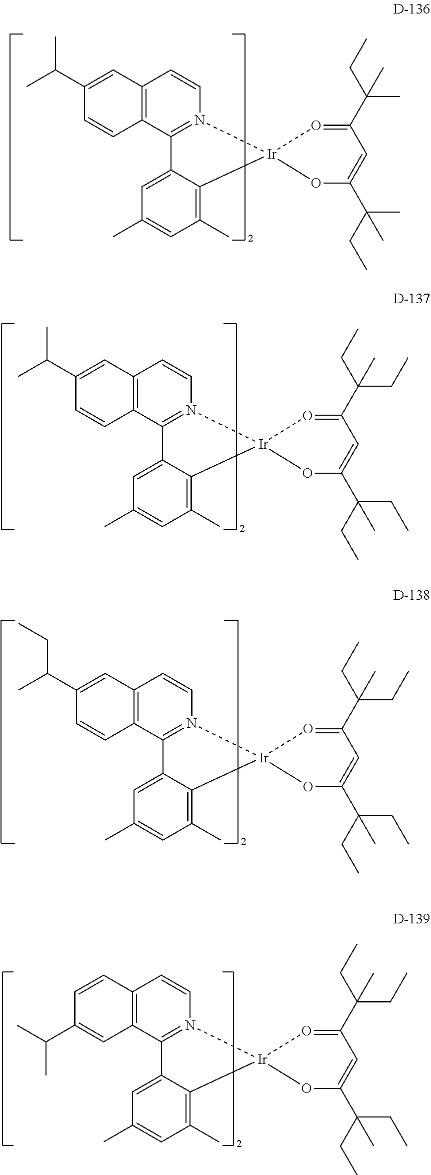

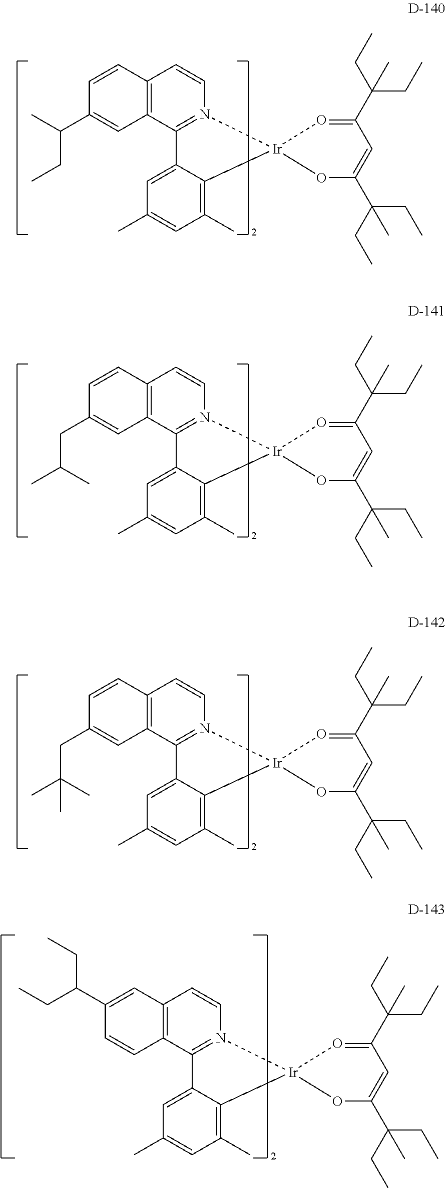

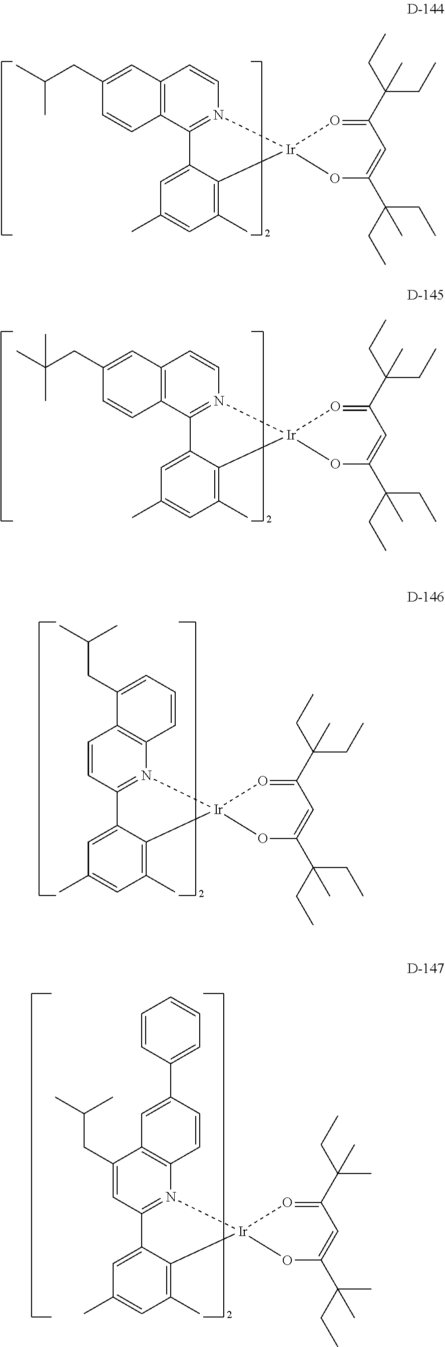

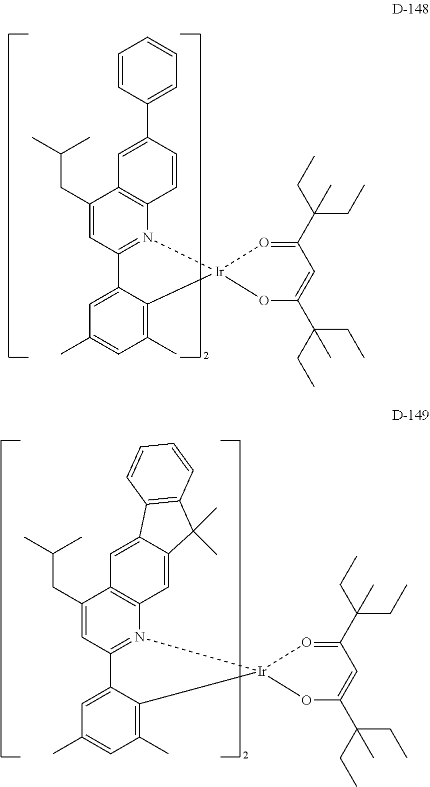

In addition, the present disclosure provides an organic electroluminescent material comprising the organic electroluminescent compound represented by formula 4, and an organic electroluminescent device comprising the material. The material may consist of the organic electroluminescent compound of the present disclosure alone, or may further comprise conventional materials contained in an organic electroluminescent material.

The organic electroluminescent compound of formula 4 of the present disclosure may be comprised in at least one layer of the light-emitting layer, the hole injection layer, the hole transport layer, the hole auxiliary layer, the light-emitting auxiliary layer, the electron transport layer, the electron buffer layer, the electron injection layer, the interlayer, the hole blocking layer, and the electron blocking layer, preferably in at least one layer of the light-emitting layer, the hole transport layer, the hole auxiliary layer, the light-emitting auxiliary layer, the electron transport layer, the electron buffer layer, the hole blocking layer, and the electron blocking layer. When used in the light-emitting layer, the organic electroluminescent compound of formula 4 of the present disclosure may be comprised as a host material. If necessary, the organic electroluminescent compound of the present disclosure may be used as a co-host material.

An organic electroluminescent device according to the present disclosure has an anode, a cathode, and at least one organic layer between the anode and the cathode. The organic layer comprises a light-emitting layer and may further comprise at least one layer selected from a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer. Each of the layers may be further configured as a plurality of layers.

The anode and the cathode may be respectively formed with a transparent conductive material, or a transflective or reflective conductive material. The organic electroluminescent device may be a top emission type, a bottom emission type, or a both-sides emission type, depending on the materials forming the anode and the cathode. In addition, the hole injection layer may be further doped with a p-dopant, and the electron injection layer may be further doped with an n-dopant.

The organic layer may further comprise at least one compound selected from the group consisting of arylamine-based compounds and styrylarylamine-based compounds.

Further, in the organic electroluminescent device of the present disclosure, the organic layer may further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising the metal.

In addition, the organic electroluminescent device of the present disclosure may emit white light by further comprising at least one light-emitting layer, which comprises a blue, a red, or a green electroluminescent compound known in the field, besides the compound of the present disclosure. If necessary, it may further comprise a yellow or an orange light-emitting layer.

In the organic electroluminescent device of the present disclosure, preferably, at least one layer selected from a chalcogenide layer, a metal halide layer, and a metal oxide layer (hereinafter, “a surface layer”) may be placed on an inner surface(s) of one or both electrode(s). Specifically, a chalcogenide (including oxides) layer of silicon or aluminum is preferably placed on an anode surface of an electroluminescent medium layer, and a metal halide layer or a metal oxide layer is preferably placed on a cathode surface of an electroluminescent medium layer. Such a surface layer provides operation stability for the organic electroluminescent device. Preferably, the chalcogenide includes SiO_{X} (1≤X≤2), AlO_{X} (1≤X≤1.5), SiON, SiAlON, etc.; the metal halide includes LiF, MgF₂, CaF₂, a rare earth metal fluoride, etc.; and the metal oxide includes Cs₂O, Li₂O, MgO, SrO, BaO, CaO, etc.

A hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. The hole transport layer or the electron blocking layer may also be multi-layers.

An electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole blocking layer or the electron transport layer may also be multi-layers, wherein each of the multi-layers may use a plurality of compounds.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or hole transport, or for preventing the overflow of electrons. When the light-emitting auxiliary layer is placed between the cathode and the light-emitting layer, it can be used for promoting the electron injection and/or electron transport, or for preventing the overflow of holes. Also, the hole auxiliary layer may be placed between the hole transport layer (or hole injection layer) and the light-emitting layer, and may be effective to promote or block the hole transport rate (or hole injection rate), thereby enabling the charge balance to be controlled. Further, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. When an organic electroluminescent device includes two or more hole transport layers, the hole transport layer, which is further included, may be used as a hole auxiliary layer or an electron blocking layer. The light-emitting auxiliary layer, the hole auxiliary layer or the electron blocking layer may have an effect of improving the efficiency and/or the lifetime of the organic electroluminescent device.

Preferably, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to the light-emitting medium. Furthermore, the hole transport compound is oxidized to a cation, and thus it becomes easier to inject and transport holes from the mixed region to the light-emitting medium. Preferably, the oxidative dopant includes various Lewis acids and acceptor compounds; and the reductive dopant includes alkali metals, alkali metal compounds, alkaline earth metals, rare-earth metals, and mixtures thereof. The reductive dopant layer may be employed as a charge-generating layer to prepare an organic electroluminescent device having two or more light-emitting layers and emitting white light.

The organic electroluminescent material according to the present disclosure may be used as a light-emitting material for a white organic light-emitting device. The white organic light-emitting device has been suggested to have various structures such as a side-by-side structure or a stacking structure depending on the arrangement of R (red), G (green) or YG (yellow green), and B (blue) light-emitting parts, or color conversion material (CCM) method, etc. The organic electroluminescent material according to the present disclosure may also be used in an organic electroluminescent device comprising a quantum dot (QD).

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used. When the first and second host compounds of the present disclosure are used to form a film, a co-evaporation process or a mixture-evaporation process is carried out.

When using a wet film-forming method, a thin film can be formed by dissolving or diffusing materials forming each layer into any suitable solvent such as ethanol, chloroform, tetrahydrofuran, dioxane, etc. The solvent can be any one where the materials forming each layer can be dissolved or diffused, and where there are no problems in film-formation capability.

In addition, it is possible to produce a display system, for example, a display system for smart phones, tablets, notebooks, PCs, TVs, or cars; or a lighting system, for example an outdoor or indoor lighting system, by using the organic electroluminescent device of the present disclosure.

Hereinafter, the preparation method of the compounds according to the present disclosure and the properties thereof will be explained in detail with reference to the representative compounds of the present disclosure. However, the present disclosure is not limited by the following examples.

### Example 1: Preparation of Compound C-6

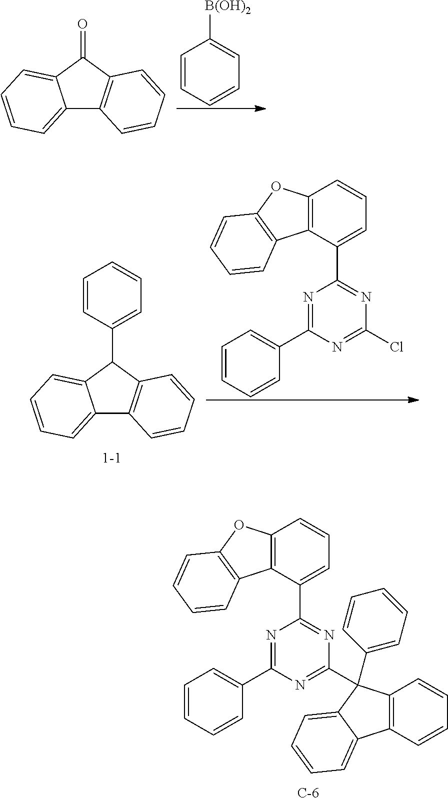

Synthesis of Compound 1-1

In a flask, 9H-fluoren-9-on (10 g, 55 mmol) and p-toluenesulfonyl hydrazide (15.5 g, 83 mmol) were dissolved in 550 mL of toluene, and then the mixture was stirred at 80° C. for 2 hours. Thereafter, phenylboronic acid (10.1 g, 83 mmol) and potassium carbonate (15.3 g, 110 mmol) were added to the mixture, and refluxed at 110° C. for 5 hours. After completion of the reaction, the organic layer was extracted with ethyl acetate, and residual moisture was removed with magnesium sulfate. The residue was dried and separated by column chromatography to obtain compound 1-1 (9.7 g, yield: 72%).

Synthesis of Compound C-6

In a flask, compound 1-1 (5.0 g, 20.6 mmol) was dissolved in 200 mL of tetrahydrofuran (THF), and 2.5 M n-BuLi in hexane (10.7 mL, 26.8 mmol) was slowly added dropwise thereto under nitrogen atmosphere at −78° C. After 2 hours, 2-chloro-4-(dibenzo[b,d]furan-1-yl)-6-phenyl-1,3,5-triazine (9.6 g, 26.8 mmol) was added to the mixture, and stirred at room temperature for 3 hours. After completion of the reaction, the organic layer was extracted with ethyl acetate, and residual moisture was removed with magnesium sulfate. The residue was dried and separated by column chromatography to obtain compound C-6 (8 g, yield: 69%).

| Compound | MW | M.P. | Tg |
|---|---|---|---|
| C-6 | 563.66 | 244° C. | 103.7° C. |

### Example 2: Preparation of Compound C-5

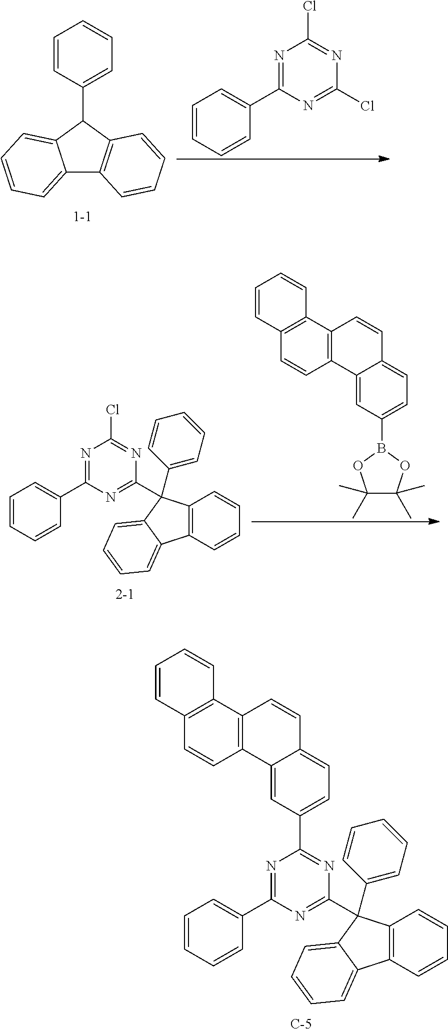

Synthesis of Compound 2-1

In a flask, compound 1-1 (4.7 g, 19 mmol) was dissolved in 190 mL of THF, and 2.5 M n-BuLi in hexane (10 mL, 25 mmol) was slowly added dropwise thereto under nitrogen atmosphere at −78° C. After 2 hours, 2,4-dichloro-6-phenyl-1,3,5-triazine (5.7 g, 25 mmol) was added to the mixture, and stirred at room temperature for 3 hours. After completion of the reaction, the organic layer was extracted with ethyl acetate, and residual moisture was removed with magnesium sulfate. The residue was dried and separated by column chromatography to obtain compound 2-1 (4.7 g, yield: 56%).

Synthesis of Compound C-5

In a flask, compound 2-1 (4.2 g, 9.7 mmol), 2-(chrysen-3-yl)-4,4,5,5-tetramethyl-1,3,2-dioxaborolane (4.1 g, 11.6 mmol), tetrakis(triphenylphosphine)palladium(0) (0.56 g, 0.48 mmol), and potassium carbonate (3.4 g, 24 mmol) were dissolved in 48 mL of toluene, 12 mL of ethanol, and 12 mL of water, and the mixture was refluxed for 2.5 hours. After completion of the reaction, the organic layer was extracted with ethyl acetate, and residual moisture was removed with magnesium sulfate. The residue was dried and separated by column chromatography to obtain compound C-5 (2.5 g, yield: 41%).

| Compound | MW | M.P. | Tg |
|---|---|---|---|
| C-5 | 623.76 | 355.6° C. | 137.99° C. |

### Example 3: Preparation of Compound C-145

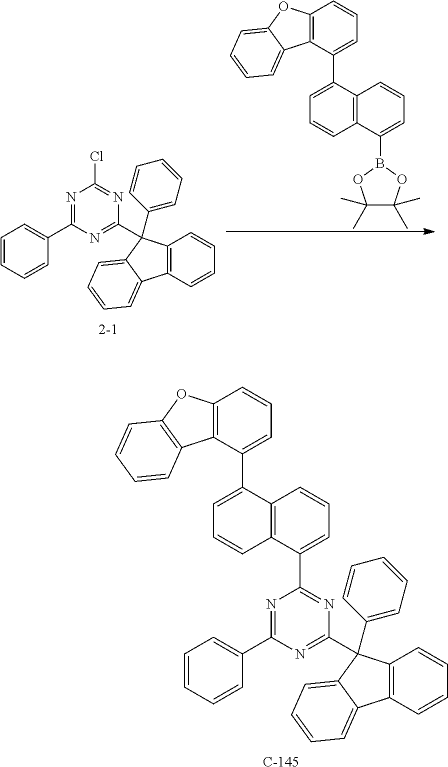 [SMILES: Clc1nc(-c2ccccc2)nc(C2(c3ccccc3)c3ccccc3-c3ccccc32)n1 ; CC1(C)OB(c2cccc3c(-c4cccc5oc6ccccc6c45)cccc23)OC1(C)C ; c1ccc(-c2nc(-c3cccc4c(-c5cccc6oc7ccccc7c56)cccc34)nc(C3(c4ccccc4)c4ccccc4-c4ccccc43)n2)cc1 ; "C-145"]

In a flask, compound 2-1 (4.6 g, 11 mmol), 2-(5-(dibenzo[b,d]furan-1-yl)naphthalen-1-yl)-4,4,5,5-tetramethyl-1,3,2-dioxaborolane (5.0 g, 12 mmol), tetrakis(triphenylphosphine)palladium(0) (0.62 g, 0.5 mmol), and potassium carbonate (3.7 g, 27 mmol) were dissolved in 56 mL of toluene, 14 mL of ethanol, and 14 mL of water, and the mixture was refluxed for 3.5 hours. After completion of the reaction, the organic layer was extracted with ethyl acetate, and residual moisture was removed with magnesium sulfate. The residue was dried and separated by column chromatography to obtain compound C-145 (2.6 g, yield: 35%).

| Compound | MW | M.P. | Tg |
|---|---|---|---|
| C-145 | 689.82 | 273° C. | 136.54° C. |

### Example 4: Preparation of Compound C-26

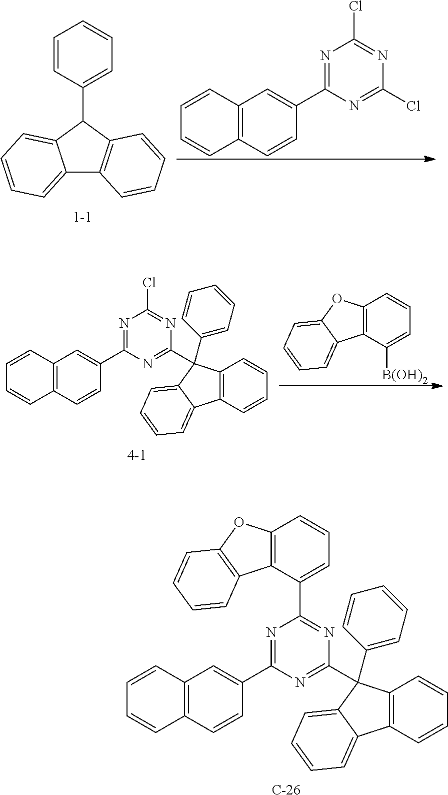

Synthesis of Compound 4-1

In a flask, compound 1-1 (15 g, 62 mmol) was dissolved in 800 mL of THF, and 2.5 M n-BuLi in hexane (32 mL, 80 mmol) was slowly added dropwise thereto under nitrogen atmosphere at −78° C. After 2 hours, 2,4-dichloro-6-(naphthalen-2-yl)-1,3,5-triazine (22.2 g, 80 mmol) was added to the mixture, and stirred at room temperature for 18 hours. After completion of the reaction, the organic layer was extracted with ethyl acetate, and residual moisture was removed with magnesium sulfate. The residue was dried and separated by column chromatography to obtain compound 4-1 (7.0 g, yield: 23%).

Synthesis of Compound C-26

In a flask, compound 4-1 (3.0 g, 6.2 mmol), dibenzofuran-1-boronic acid (1.5 g, 6.8 mmol), tetrakis(triphenylphosphine)palladium(0) (0.36 g, 0.31 mmol), and potassium carbonate (2.1 g, 15 mmol) were dissolved in 32 mL of toluene, 8 mL of ethanol, and 8 mL of water, and the mixture was refluxed for 2.5 hours. After completion of the reaction, the organic layer was extracted with ethyl acetate, and residual moisture was removed with magnesium sulfate. The residue was dried and separated by column chromatography to obtain compound C-26 (2.0 g, yield: 52%).

| Compound | MW | M.P. | Tg |
|---|---|---|---|
| C-26 | 613.72 | 212° C. | 115.76° C. |

### Example 5: Preparation of Compound C-101

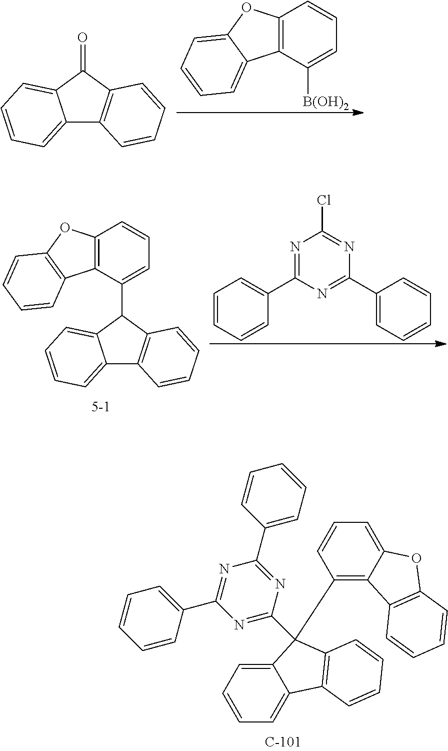

Synthesis of compound 5-1

In a flask, 9H-fluoren-9-on (10 g, 55 mmol) and p-toluenesulfonyl hydrazide (15.5 g, 83 mmol) were dissolved in 550 mL of toluene, and then the mixture was stirred at 80° C. for 2 hours. Thereafter, dibenzofuran-1-boronic acid (17.6 g, 83 mmol) and potassium carbonate (15.3 g, 110 mmol) were added to the mixture, and refluxed at 110° C. for 5 hours. After completion of the reaction, the organic layer was extracted with ethyl acetate, and residual moisture was removed with magnesium sulfate. The residue was dried and separated by column chromatography to obtain compound 5-1 (7 g, yield: 38%).

Synthesis of Compound C-101

In a flask, compound 5-1 (7.5 g, 20.6 mmol) was dissolved in 220 mL of THF, and 2.5 M n-BuLi in hexane (11.6 mL, 29 mmol) was slowly added dropwise thereto under nitrogen atmosphere at −78° C. After 2 hours, 2-chloro-4,6-diphenyl-1,3,5-triazine (7.8 g, 29 mmol) was added to the mixture, and stirred at room temperature for 3 hours. After completion of the reaction, the organic layer was extracted with ethyl acetate, and residual moisture was removed with magnesium sulfate. The residue was dried and separated by column chromatography to obtain compound C-101 (7.9 q, yield: 62%).

| Compound | MW | M.P. | Tg |
|---|---|---|---|
| C-101 | 563.66 | 251.6° C. | 127.42° C. |

### Example 6: Preparation of Compound C-66

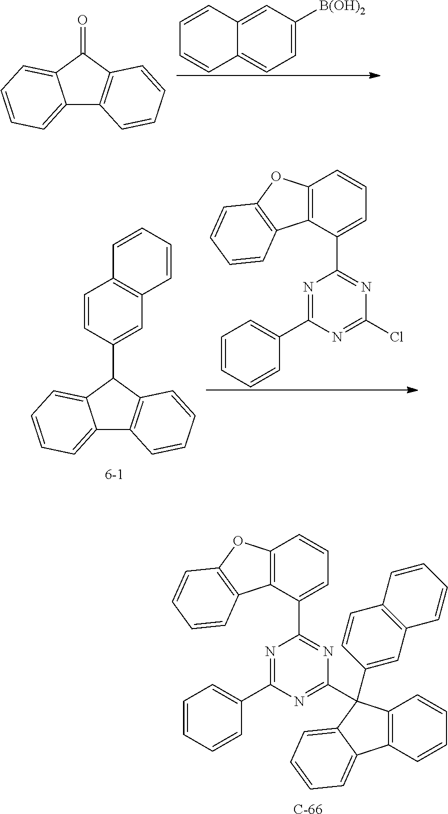

Synthesis of Compound 6-1

In a flask, 9H-fluoren-9-on (10 g, 55 mmol) and p-toluenesulfonyl hydrazide (15.5 g, 83 mmol) were dissolved in 550 mL of toluene, and then the mixture was stirred at 80° C. for 2 hours. Thereafter, naphthalen-2-ylboronic acid (14 g, 83 mmol) and potassium carbonate (15.3 g, 110 mmol) were added to the mixture, and refluxed at 110° C. for 3 hours. After completion of the reaction, the organic layer was extracted with ethyl acetate, and residual moisture was removed with magnesium sulfate. The residue was dried and separated by column chromatography to obtain compound 6-1 (9.0 g, yield: 56%).

Synthesis of Compound C-66

In a flask, compound 6-1 (4.0 g, 13.7 mmol) was dissolved in 137 mL of THF, and 2.5 M n-BuLi in hexane (7.1 mL, 17.7 mmol) was slowly added dropwise thereto under nitrogen atmosphere at −78° C. After 2 hours, 2-chloro-4-(dibenzo[b,d]furan-1-yl)-6-phenyl-1,3,5-triazine (6.4 g, 17.7 mmol) was added to the mixture, and stirred at room temperature for 3 hours. After completion of the reaction, the organic layer was extracted with ethyl acetate, and residual moisture was removed with magnesium sulfate. The residue was dried and separated by column chromatography to obtain compound C-66 (1.8 g, yield: 21%).

| Compound | MW | M.P. | Tg |
|---|---|---|---|
| C-66 | 613.72 | 195.8° C. | 115.72° C. |

### Example 7: Preparation of Compound C-161

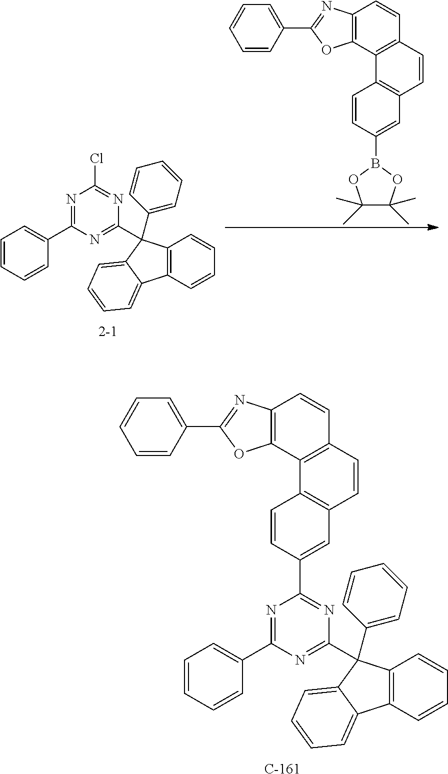 [SMILES: Clc1nc(-c2ccccc2)nc(C2(c3ccccc3)c3ccccc3-c3ccccc32)n1 ; CC1(C)OB(c2ccc3c(ccc4ccc5nc(-c6ccccc6)oc5c43)c2)OC1(C)C ; c1ccc(-c2nc(-c3ccc4c(ccc5ccc6nc(-c7ccccc7)oc6c54)c3)nc(C3(c4ccccc4)c4ccccc4-c4ccccc43)n2)cc1 ; "C-161"]

In a flask, compound 2-1 (3.0 g, 7 mmol), 2-phenyl-9-(4,4,5,5-tetramethyl-1,3,2-dioxaborolan-2-yl)phenanthro[3,4-d]oxazole (2.9 g, 7 mmol), tetrakis(triphenylphosphine)palladium(0) (0.4 g, 0.3 mmol), and potassium carbonate (2.4 g, 17 mmol) were dissolved in 34 mL of toluene, 8.5 mL of ethanol, and 8.5 mL of water, and the mixture was refluxed for 2.5 hours. After completion of the reaction, the organic layer was extracted with ethyl acetate, and residual moisture was removed with magnesium sulfate. The residue was dried and separated by column chromatography to obtain compound C-161 (3.0 g, yield: 53%).

| Compound | MW | M.P. | Tg |
|---|---|---|---|
| C-161 | 690.81 | 332° C. | 161° C. |

### Example 8: Preparation of Compound C-151

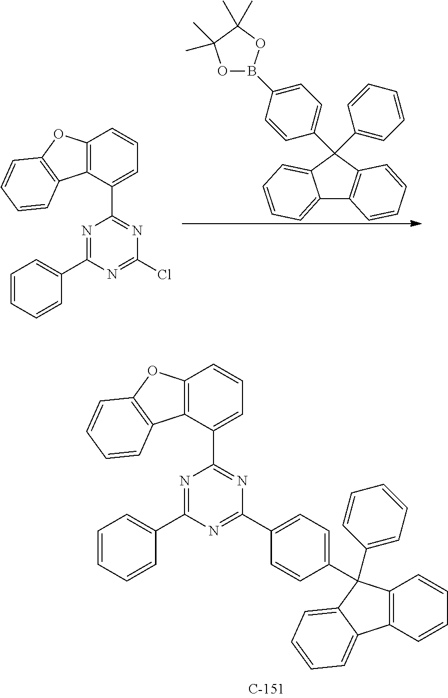 [SMILES: Clc1nc(-c2ccccc2)nc(-c2cccc3oc4ccccc4c23)n1 ; CC1(C)OB(c2ccc(C3(c4ccccc4)c4ccccc4-c4ccccc43)cc2)OC1(C)C ; c1ccc(-c2nc(-c3ccc(C4(c5ccccc5)c5ccccc5-c5ccccc54)cc3)nc(-c3cccc4oc5ccccc5c34)n2)cc1]

In a flask, 2-chloro-4-(dibenzo[b,d]furan-1-yl)-6-phenyl-1,3,5-triazine (10 g, 28 mmol), 4,4,5,5-tetramethyl-2-(4-(9-phenyl-9H-fluoren-9-yl)phenyl)-1,3,2-dioxaborolane (15.0 g, 33 mmol), tetrakis(triphenylphosphine)palladium(0) (1.6 g, 1.4 mmol), and potassium carbonate (9.6 g, 70 mmol) were dissolved in 140 mL of toluene, 35 mL of ethanol, and 35 mL of water, and the mixture was refluxed for 2.5 hours. After completion of the reaction, the organic layer was extracted with ethyl acetate, and residual moisture was removed with magnesium sulfate. The residue was dried and separated by column chromatography to obtain compound C-151 (8.5 g, yield: 47%).

| Compound | MW | M.P. | Tg |
|---|---|---|---|
| C-151 | 639.76 | 172.3° C. | 130.42° C. |

### Example 9: Preparation of Compound C-194

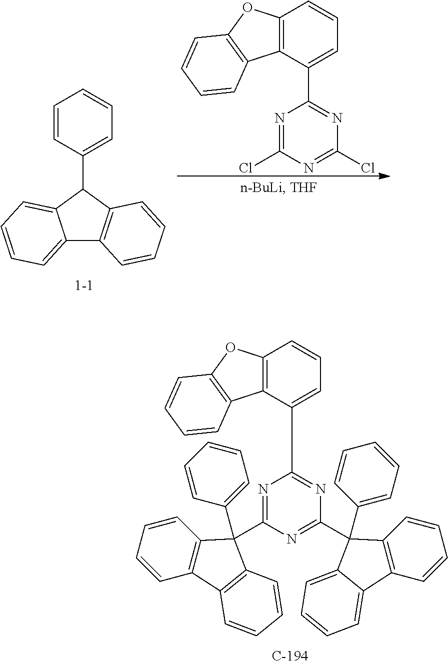

In a flask, compound 1-1 (10 g, 41 mmol) was dissolved in 410 mL of THF, and 2.5 M n-BuLi in hexane (21.6 mL, 54 mmol) was slowly added dropwise thereto under nitrogen atmosphere at −78° C. After 2 hours, 2,4-dichloro-6-(dibenzo[b,d]furan-1-yl)-1,3,5-triazine (17 g, 53.6 mmol) was added to the mixture, and stirred at room temperature for 18 hours. After completion of the reaction, the organic layer was extracted with ethyl acetate, and residual moisture was removed with magnesium sulfate. The residue was dried and separated by column chromatography to obtain compound C-194 (6.2 g, yield: 21%).

| Compound | MW | M.P. | Tg |
|---|---|---|---|
| C-194 | 727.87 | 255° C. | 117.48° C. |

### Example 10: Preparation of Compound C-222

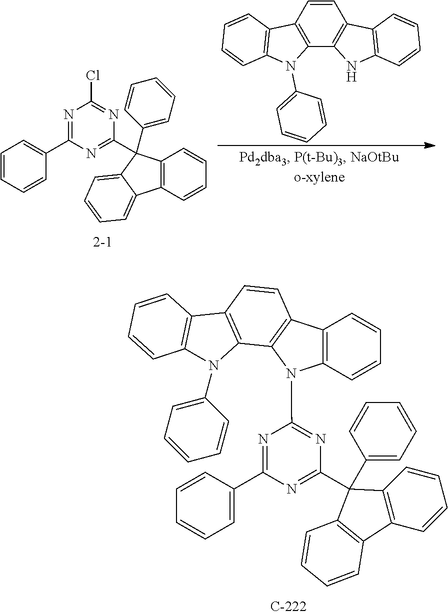 [SMILES: Clc1nc(-c2ccccc2)nc(C2(c3ccccc3)c3ccccc3-c3ccccc32)n1 ; c1ccc(-n2c3ccccc3c3ccc4c5ccccc5[nH]c4c32)cc1 ; c1ccc(-c2nc(-n3c4ccccc4c4ccc5c6ccccc6n(-c6ccccc6)c5c43)nc(C3(c4ccccc4)c4ccccc4-c4ccccc43)n2)cc1]

In a flask, compound 2-1 (10.4 g, 24 mmol), 11-phenyl-11,12-dihydroindolo[2,3-a]carbazole (4.0 g, 12 mmol), Pd₂dba₃ (0.55 g, 0.6 mmol), P(t-Bu)₃ (0.6 mL, 1.2 mmol), and NaOtBu (2.9 g, 30 mmol) were dissolved in 120 mL of o-xylene, and the mixture was refluxed for 1 hour. After completion of the reaction, the organic layer was extracted with ethyl acetate, and residual moisture was removed with magnesium sulfate. The residue was dried and separated by column chromatography to obtain compound C-222 (5.2 g, yield: 60%).

| Compound | MW | M.P. | Tg |
|---|---|---|---|
| C-222 | 727.87 | 243° C. | 155.35° C. |

Hereinafter, a method of producing an organic electroluminescent device (OLED) according to the present disclosure and the luminous efficiency and lifetime properties thereof will be explained in detail. However, the present disclosure is not limited by the following examples.

### Device Examples 1 to 9: Producing an OLED According to the Present Disclosure

An OLED according to the present disclosure was produced. A transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropanol. The ITO substrate was then mounted on a substrate holder of a vacuum vapor deposition apparatus. Compound HI-1 was introduced into a cell of the vacuum vapor deposition apparatus, and compound HT-1 was introduced into another cell of the vacuum vapor deposition apparatus. The two materials were evaporated at different rates, and compound HI-1 was deposited in a doping amount of 3 wt % based on the total amount of compound HI-1 and compound HT-1 to form a hole injection layer having a thickness of 10 nm on the ITO substrate. Next, compound HT-1 was deposited on the hole injection layer to form a first hole transport layer having a thickness of 80 nm. Compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was formed thereon as follows: the first and second host materials shown in Table 1 below were introduced into two cells of the vacuum vapor deposition apparatus as hosts, and compound D-71 was introduced into another cell as a dopant. The two host materials were evaporated at a rate of 1:1 and the dopant material was simultaneously evaporated at a different rate, and the dopant was deposited in a doping amount of 3 wt % based on the total amount of the host and the dopant to form a light-emitting layer having a thickness of 40 nm on the second hole transport layer. Compound ETL-1 and compound EIL-1 were evaporated in a weight ratio of 50:50 to form an electron transport layer having a thickness of 35 nm on the light-emitting layer. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus, an OLED was produced. All the materials used for producing the OLED were purified by vacuum sublimation at 10⁻⁶ torr.

### Comparative Example 1: Producing an OLED Comprising a Comparative Compound as a Host

An OLED was produced in the same manner as in Device Example 1, except that the second host compound shown in Table 1 below was used alone as a host of the light-emitting layer.

The driving voltage, luminous efficiency, and light-emitting color at a luminance of 1,000 nit, and the time taken for luminance to decrease from 100% to 95% at a luminance of 5,000 nit (lifetime; T95) of the OLEDs produced in Comparative Example 1 and Device Examples 1 to 9 are provided in Table 1 below.

TABLE 1

| | | | | | Light- | Life- |
|---|---|---|---|---|---|---|
| | | | Driving | Luminous | Emit- | time |
| | First | Second | Voltage | Efficiency | ting | (T95) |
| | Host | Host | [V] | [cd/A] | Color | [hr] |
| Comparative | — | CBP | 9.0 | 14.3 | Red | 0.31 |
| Example 1 | | | | | | |
| Device | C-6 | H-185 | 3.0 | 35.1 | Red | 203 |
| Example 1 | | | | | | |
| Device | C-101 | H-185 | 3.2 | 33.7 | Red | 49 |
| Example 2 | | | | | | |
| Device | C-5 | H-185 | 3.2 | 34.2 | Red | 250 |
| Example 3 | | | | | | |
| Device | C-26 | H-185 | 2.9 | 37.6 | Red | 256 |
| Example 4 | | | | | | |
| Device | C-145 | H-185 | 3.2 | 36.0 | Red | 136 |
| Example 5 | | | | | | |
| Device | C-151 | H-185 | 3.0 | 35.6 | Red | 115 |
| Example 6 | | | | | | |
| Device | C-66 | H-185 | 3.0 | 36.7 | Red | 119 |
| Example 7 | | | | | | |
| Device | C-161 | H-185 | 3.1 | 35.3 | Red | 137 |
| Example 8 | | | | | | |
| Device | C-194 | H-185 | 3.1 | 34.3 | Red | 125 |
| Example 9 | | | | | | |

### Device Examples 10 to 14: Producing a Green Light-Emitting OLED According to the Present Disclosure

OLEDs were produced in the same manner as in Device Example 1, except that the second hole transport layer, the light-emitting layer, and the electron transport layer were formed as follows: Compound HT-3 was introduced into another cell of the vacuum vapor deposition apparatus and was evaporated by applying an electric current to the cell, thereby forming a second hole transport layer having a thickness of 30 nm on the first hole transport layer. After forming the hole injection layer and the hole transport layers, a light-emitting layer was formed thereon as follows: the first and second host materials shown in Table 2 below were introduced into two cells of the vacuum vapor deposition apparatus as hosts, and compound PGD was introduced into another cell as a dopant. The two host materials were evaporated at a rate of 2:1 and the dopant material was simultaneously evaporated at a different rate, and the dopant was deposited in a doping amount of 10 wt % based on the total amount of the hosts and the dopant to form a light-emitting layer having a thickness of 40 nm on the second hole transport layer. Compound ETL-1 and compound EIL-1 were evaporated in a weight ratio of 40:60 to form an electron transport layer having a thickness of 35 nm on the light-emitting layer.

The driving voltage, luminous efficiency, and light-emitting color at a luminance of 1,000 nit of the OLEDs produced in Device Examples 10 to 14 are provided in Table 2 below.

TABLE 2

| | | | Driving | Luminous | Light- |
|---|---|---|---|---|---|
| | | | Voltage | Efficiency | Emitting |
| | First Host | Second Host | [V] | (cd/A) | Color |
| Device | C-6 | H2-6 | 3.1 | 101.1 | Green |
| Example 10 | | | | | |
| Device | C-101 | H2-6 | 3.1 | 104.9 | Green |
| Example 11 | | | | | |
| Device | C-194 | H2-6 | 3.3 | 101.5 | Green |
| Example 12 | | | | | |
| Device | C-151 | H2-6 | 3.1 | 103.6 | Green |
| Example 13 | | | | | |
| Device | C-222 | H2-6 | 3.5 | 100.6 | Green |
| Example 14 | | | | | |

From Tables 1 and 2 above, it can be confirmed that the OLEDs comprising a specific combination of compounds according to the present disclosure as host materials exhibit low driving voltage, high luminous efficiency, and/or improved lifetime properties compared to the OLED using the conventional compound as a single host material (Comparative Example 1). That is, it can be confirmed that the organic electroluminescent compounds of the present disclosure exhibit superior light-emitting properties to the conventional material. In addition, it can be seen that the OLED using the compound for an organic electroluminescent material according to the present disclosure as a host material(s) for emitting light shows excellent luminous efficiency properties.

The compounds used in the Device Examples and the Comparative Example are shown in Table 3.

TABLE 3

| | |
|---|---|
| Hole Injection Layer/ Hole Transport Layer | 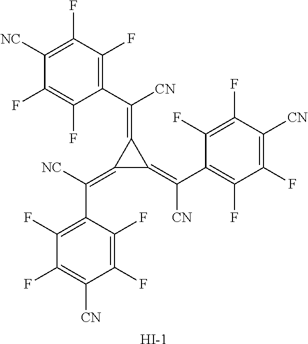 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 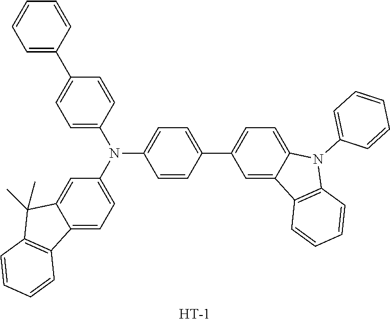 |
| | 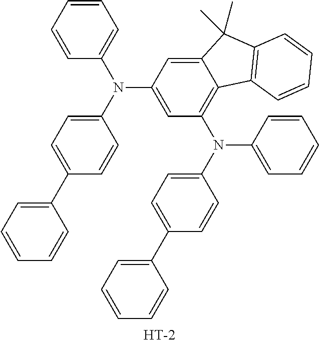 |
| | 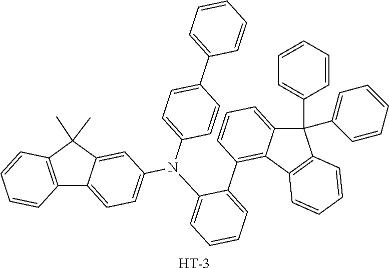 |
| Light- Emitting Layer | 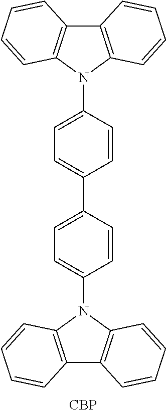 |
| | 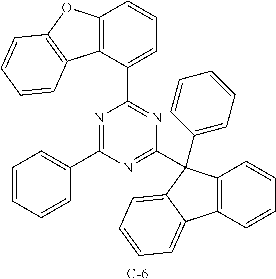 |
| | 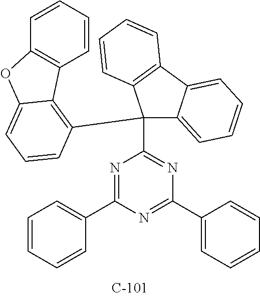 [SMILES: c1ccc(-c2nc(-c3ccccc3)nc(C3(c4cccc5oc6ccccc6c45)c4ccccc4-c4ccccc43)n2)cc1] |
| | 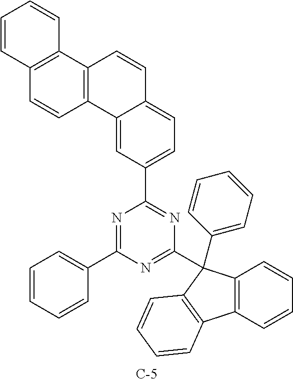 |
| | 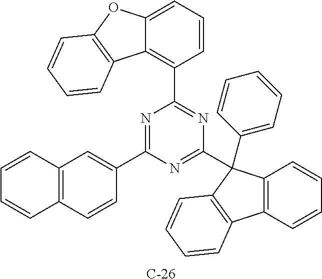 |
| | 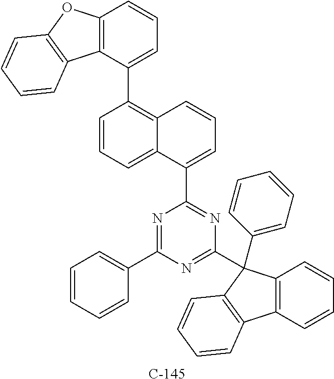 |
| | 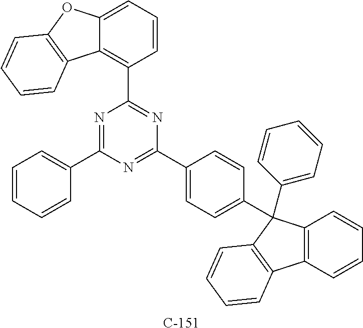 [SMILES: c1ccc(-c2nc(-c3ccc(C4(c5ccccc5)c5ccccc5-c5ccccc54)cc3)nc(-c3cccc4oc5ccccc5c34)n2)cc1 ; "C-151"] |
| | 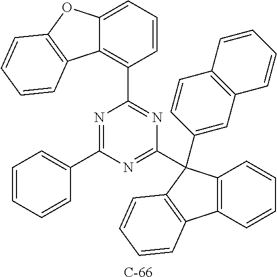 |
| | 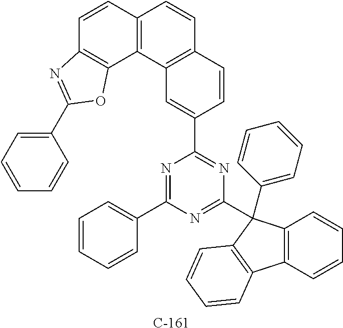 [SMILES: c1ccc(-c2nc(-c3ccc4ccc5ccc6nc(-c7ccccc7)oc6c5c4c3)nc(C3(c4ccccc4)c4ccccc4-c4ccccc43)n2)cc1 ; "C-161"] |
| | 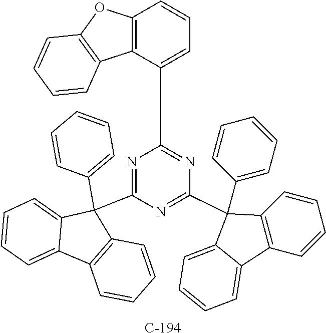 |
| | 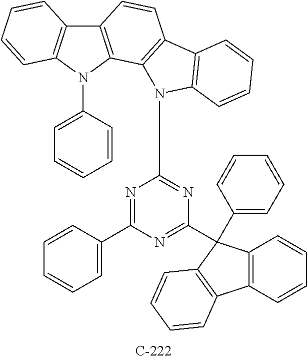 [SMILES: c1ccc(-c2nc(-n3c4ccccc4c4ccc5c6ccccc6n(-c6ccccc6)c5c43)nc(C3(c4ccccc4)c4ccccc4-c4ccccc43)n2)cc1] |
| | 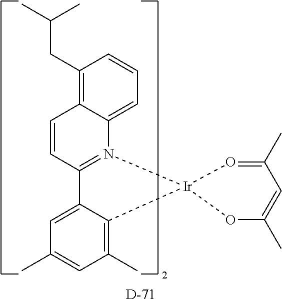 |
| | 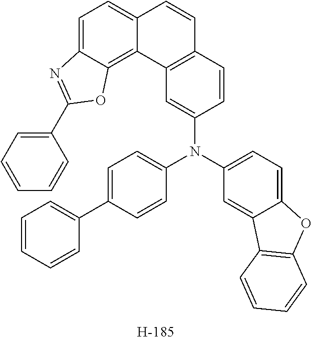 [SMILES: c1ccc(-c2ccc(N(c3ccc4oc5ccccc5c4c3)c3ccc4ccc5ccc6nc(-c7ccccc7)oc6c5c4c3)cc2)cc1] |
| | 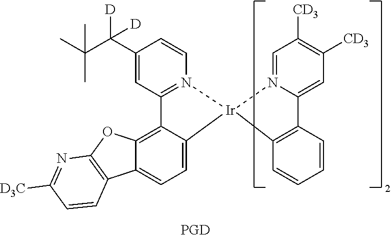 |
| | 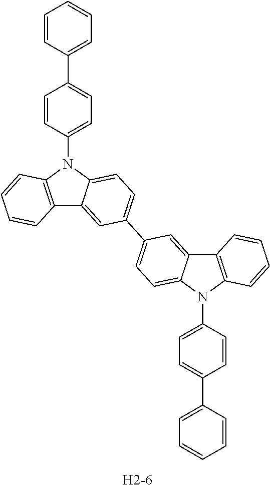 [SMILES: c1ccc(-c2ccc(-n3c4ccccc4c4cc(-c5ccc6c(c5)c5ccccc5n6-c5ccc(-c6ccccc6)cc5)ccc43)cc2)cc1] |
| Electron Transport Layer/ Electron Injection Layer | 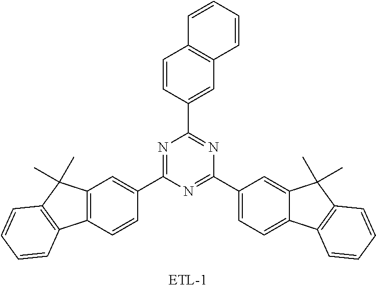 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 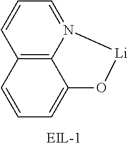 |

1. A plurality of host materials comprising a first host material comprising a compound represented by the following formula 1, and a second host material comprising a compound represented by the following formula 2 or 3: 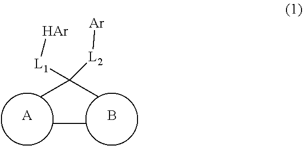
in formula 1,
ring A and ring B, each independently, represent a substituted or unsubstituted (C6-C30)arene, or a substituted or unsubstituted (3- to 30-membered)heteroarene;
L₁ and L₂, each independently, represent a single bond, a substituted or unsubstituted (C6-C30)arylene, or a substituted or unsubstituted (3- to 30-membered)heteroarylene;
Ar represents a substituted or unsubstituted (C6-C30)aryl, or a substituted or unsubstituted (3- to 30-membered)heteroaryl; and
HAr represents a substituted or unsubstituted (3- to 20-membered)heteroaryl containing a nitrogen atom(s); 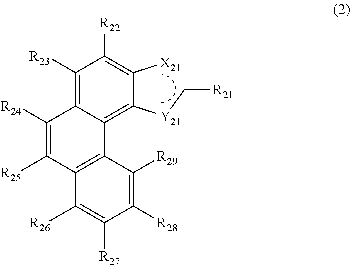
in formula 2,
X₂₁ and Y₂₁, each independently, represent —N═, —NR₃₁—, —O—, or —S—, with the proviso that any one of X₂₁ and Y₂₁ represents —N═ and the other one of X₂₁ and Y₂₁ represents —NR₃₁—, —O—, or —S—;
R₂₁ and R₃₁, each independently, represent a substituted or unsubstituted (C6-C30)aryl, or a substituted or unsubstituted (3- to 30-membered)heteroaryl;
R₂₂ to R₂₉, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), -L-NR₁R₂, or -L₂₁-Ar₂₁; or may be linked to an adjacent substituent to form a ring(s); with the proviso that at least one of R₂₂ to R₂₉ represents -L₂₁-Ar₂₁;
L₂₁, each independently, represents a single bond, a substituted or unsubstituted (C6-C30)arylene, or a substituted or unsubstituted (3- to 30-membered)heteroarylene;
Ar₂₁, each independently, represents a substituted or unsubstituted fused ring group of a (C3-C30) aliphatic ring(s) and a (C6-C30) aromatic ring(s), a substituted or unsubstituted (C6-C30)aryl, a substituted or unsubstituted (3- to 30-membered)heteroaryl, or —NR₃₂R₃₃;
R₃₂ and R₃₃, each independently, represent a substituted or unsubstituted (C1-C30)alkyl, a substituted or unsubstituted (C2-C30)alkenyl, a substituted or unsubstituted (C6-C30)aryl, a substituted or unsubstituted (3- to 30-membered)heteroaryl, or a substituted or unsubstituted fused ring group of a (C3-C30) aliphatic ring(s) and a (C6-C30) aromatic ring(s);
L represents a single bond, a substituted or unsubstituted (C6-C30)arylene, a substituted or unsubstituted (3- to 30-membered)heteroarylene, a substituted or unsubstituted divalent (C2-C30) aliphatic hydrocarbon group, or a substituted or unsubstituted divalent fused ring group of a (C3-C30) aliphatic ring(s) and a (C6-C30) aromatic ring(s); and
R₁ and R₂, each independently, represent hydrogen, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl; 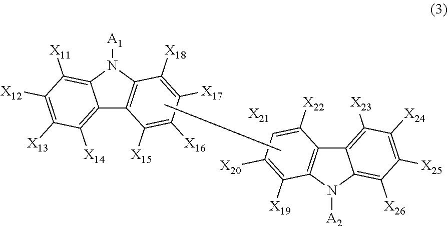
in formula 3,
A₁ and A₂, each independently, represent a substituted or unsubstituted (C6-C30)aryl, a substituted or unsubstituted dibenzofuranyl, a substituted or unsubstituted dibenzothiophenyl, or a substituted or unsubstituted carbazolyl;
one of X₁₅ to X₁₆ and one of X₁₉ to Xi are linked to each other to form a single bond;
the remaining X₁₅ to X₂₂ which do not form a single bond, X₁₁ to X₁₄, and X₂₃ to X₂₆, each independently, represent hydrogen, deuterium, a substituted or unsubstituted (C6-C30)aryl, or a substituted or unsubstituted (3- to 30-membered)heteroaryl; or may be linked to an adjacent substituent to form a ring(s).

2. The plurality of host materials according to claim 1, wherein the substituent(s) of the substituted alkyl, the substituted alkenyl, the substituted aryl, the substituted arene, the substituted arylene, the substituted heteroaryl, the substituted heteroarene, the substituted heteroarylene, the substituted dibenzofuranyl, the substituted dibenzothiophenyl, the substituted carbazolyl, the substituted cycloalkyl, the substituted alkoxy, the substituted trialkylsilyl, the substituted dialkylarylsilyl, the substituted alkyldiarylsilyl, the substituted triarylsilyl, the substituted fused ring group of an aliphatic ring(s) and an aromatic ring(s), the substituted divalent aliphatic hydrocarbon group, or the substituted divalent fused ring group of a aliphatic ring(s) and an aromatic ring(s), each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with a (C6-C30)aryl(s); a (C6-C30)aryl unsubstituted or substituted with a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a fused ring group of a (C3-C30) aliphatic ring(s) and a (C6-C30) aromatic ring(s); an amino; a mono- or di-(C1-C30)alkylamino; a mono- or di-(C2-C30)alkenylamino; a (C1-C30)alkyl(C2-C30)alkenylamino; a substituted or unsubstituted mono- or di-(C6-C30)arylamino; a (C1-C30)alkyl(C6-C30)arylamino; a mono- or di-(3- to 30-membered)heteroarylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphinyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

3. The plurality of host materials according to claim 1, wherein the formula 1 is represented by any one of the following formulas: 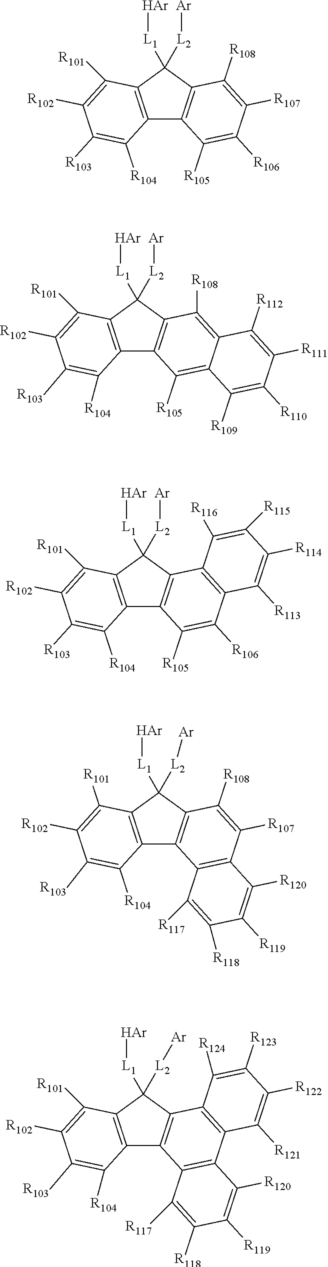 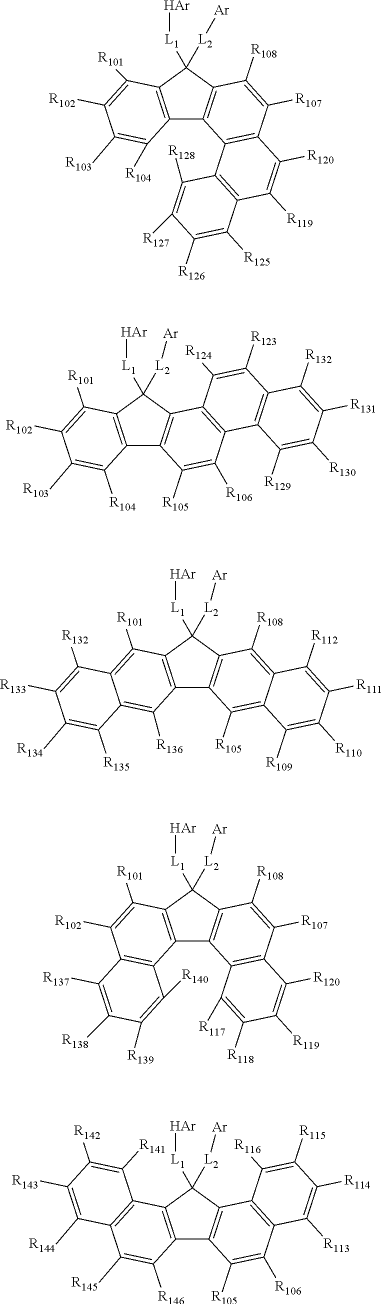 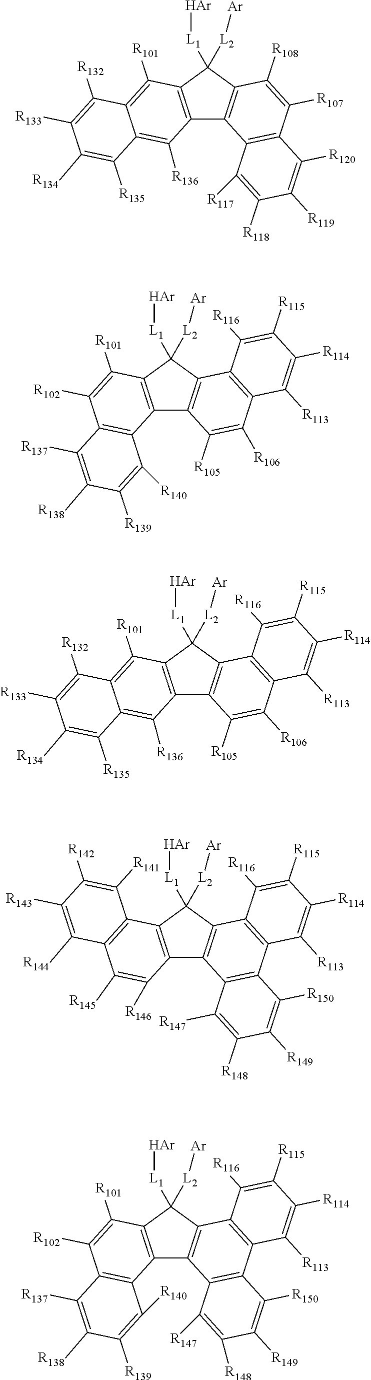 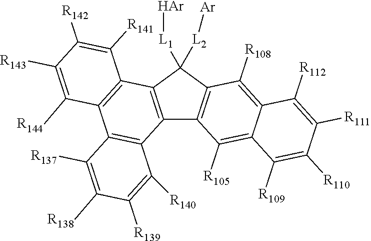
in the formulas above,
R₁₀₁ to R₁₅₀, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or -L-NR₁R₂; and
HAr, Ar, L₁, L₂, L, R₁, and R₂ are as defined in claim 1.

4. The plurality of host materials according to claim 1, wherein HAr in formula 1 represents a substituted or unsubstituted pyridyl, a substituted or unsubstituted triazinyl, a substituted or unsubstituted pyrimidinyl, a substituted or unsubstituted quinolyl, a substituted or unsubstituted quinazolinyl, a substituted or unsubstituted quinoxalinyl, a substituted or unsubstituted benzoquinazolinyl, a substituted or unsubstituted benzoquinoxalinyl, a substituted or unsubstituted benzofuropyrimidinyl, a substituted or unsubstituted carbazolyl, a substituted or unsubstituted dibenzothiophenyl, a substituted or unsubstituted benzothiophenyl, a substituted or unsubstituted dibenzofuranyl, a substituted or unsubstituted benzofuranyl, a substituted or unsubstituted naphthyridinyl, a substituted or unsubstituted benzonaphthofuranyl, or a substituted or unsubstituted benzonaphthothiophenyl.

5. The plurality of host materials according to claim 1, wherein the formula 2 is represented by any one of the following formulas: 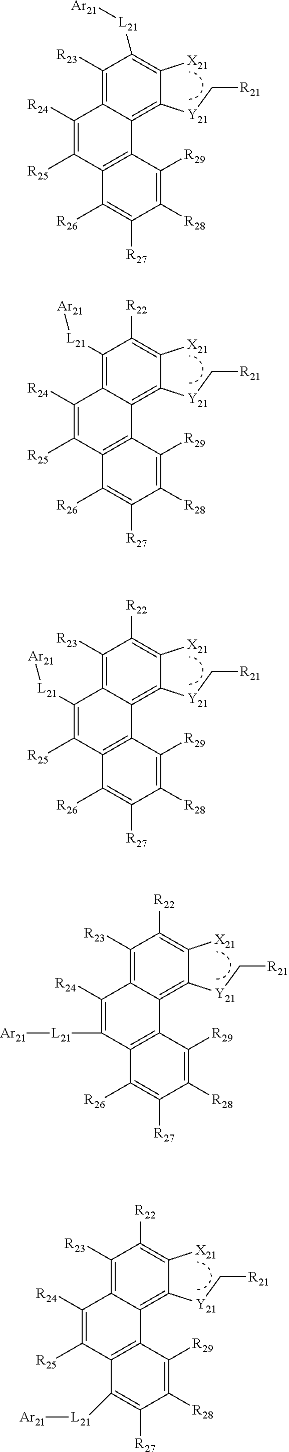 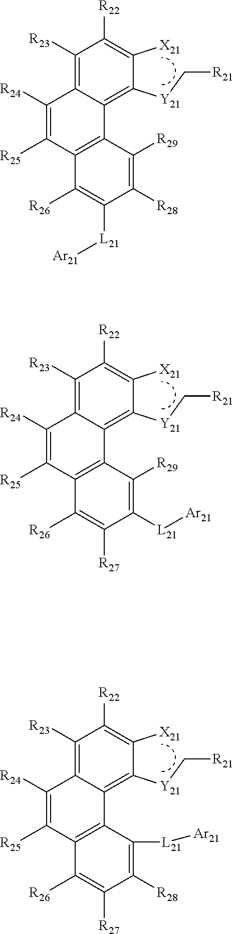
in the formulas above,
X₂₁, Y₂₁, L₂₁, Ar₂₁, and R₂₁ to R₂₉ are as defined in claim 1.

6. The plurality of host materials according to claim 1, wherein Ar₂₁ in formula 2 represents a substituted or unsubstituted phenyl, a substituted or unsubstituted naphthyl, a substituted or unsubstituted o-biphenyl, a substituted or unsubstituted m-biphenyl, a substituted or unsubstituted p-biphenyl, a substituted or unsubstituted o-terphenyl, a substituted or unsubstituted m-terphenyl, a substituted or unsubstituted p-terphenyl, a substituted or unsubstituted triphenylenyl, a substituted or unsubstituted phenanthrenyl, a substituted or unsubstituted benzo[c]phenanthrenyl, a substituted or unsubstituted chrysenyl, a substituted or unsubstituted fluoranthenyl, a substituted or unsubstituted fluorenyl, a substituted or unsubstituted benzofluorenyl, a substituted or unsubstituted spirobifluorenyl, a substituted or unsubstituted spiro[cyclopentane-fluoren]yl, a substituted or unsubstituted spiro[dihydroindene-fluoren]yl, a substituted or unsubstituted spiro[benzofluorene-fluoren]yl, a substituted or unsubstituted carbazolyl, a substituted or unsubstituted benzocarbazolyl, a substituted or unsubstituted dibenzocarbazolyl, a substituted or unsubstituted dibenzothiophenyl, a substituted or unsubstituted benzothiophenyl, a substituted or unsubstituted benzonaphthothiophenyl, a substituted or unsubstituted dibenzofuranyl, a substituted or unsubstituted benzofuranyl, or a substituted or unsubstituted benzonaphthofuranyl; or represents an amino substituted with at least one selected from the group consisting of a phenyl, a naphthyl, a naphthylphenyl, a phenylnaphthyl, an o-biphenyl, an m-biphenyl, a p-biphenyl, an o-terphenyl, an m-terphenyl, a p-terphenyl, a dimethylfluorenyl, a diphenylfluorenyl, a dimethylbenzofluorenyl, a phenanthrenyl, a dibenzothiophenyl unsubstituted or substituted with a phenyl(s), a benzonaphthofuranyl, and a dibenzofuranyl unsubstituted or substituted with a phenyl(s).

7. The plurality of host materials according to claim 1, wherein the compound represented by formula 1 is at least one selected from the following compounds: 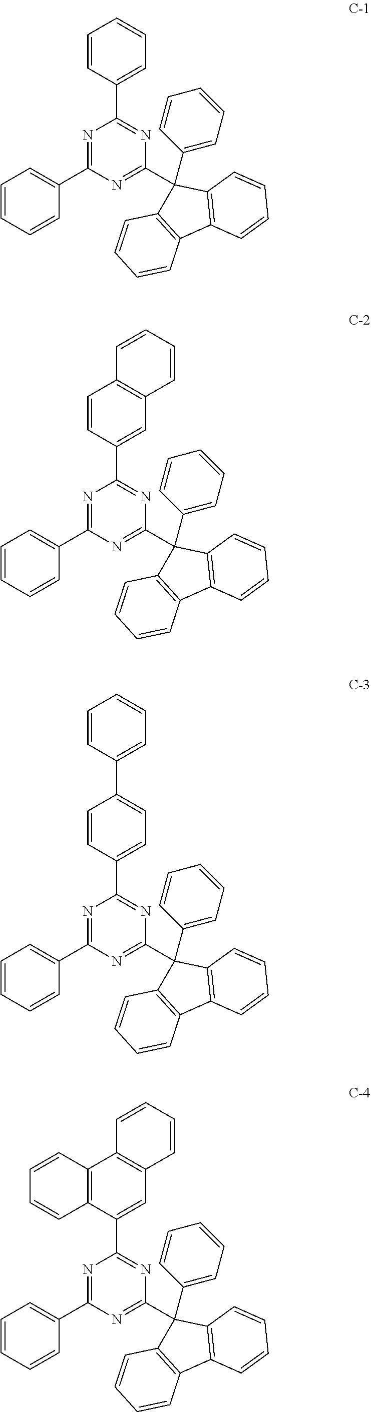 [SMILES: c1ccc(-c2nc(-c3ccccc3)nc(C3(c4ccccc4)c4ccccc4-c4ccccc43)n2)cc1 ; c1ccc(-c2nc(-c3ccc4ccccc4c3)nc(C3(c4ccccc4)c4ccccc4-c4ccccc43)n2)cc1 ; c1ccc(-c2ccc(-c3nc(-c4ccccc4)nc(C4(c5ccccc5)c5ccccc5-c5ccccc54)n3)cc2)cc1 ; c1ccc(-c2nc(-c3cc4ccccc4c4ccccc34)nc(C3(c4ccccc4)c4ccccc4-c4ccccc43)n2)cc1] 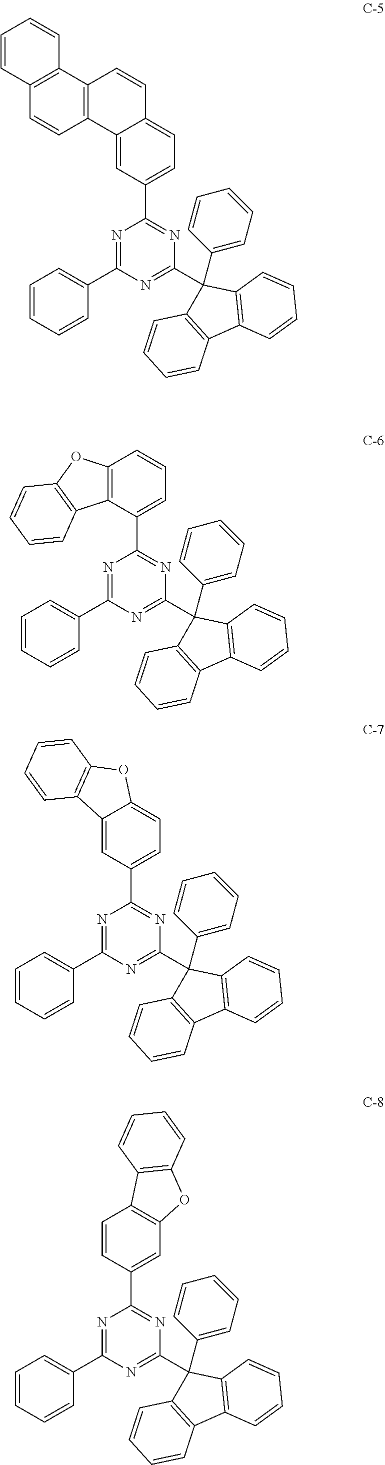 [SMILES: c1ccc(-c2nc(-c3ccc4ccc5c6ccccc6ccc5c4c3)nc(C3(c4ccccc4)c4ccccc4-c4ccccc43)n2)cc1 ; c1ccc(-c2nc(-c3cccc4oc5ccccc5c34)nc(C3(c4ccccc4)c4ccccc4-c4ccccc43)n2)cc1 ; c1ccc(-c2nc(-c3ccc4oc5ccccc5c4c3)nc(C3(c4ccccc4)c4ccccc4-c4ccccc43)n2)cc1 ; c1ccc(-c2nc(-c3ccc4c(c3)oc3ccccc34)nc(C3(c4ccccc4)c4ccccc4-c4ccccc43)n2)cc1] 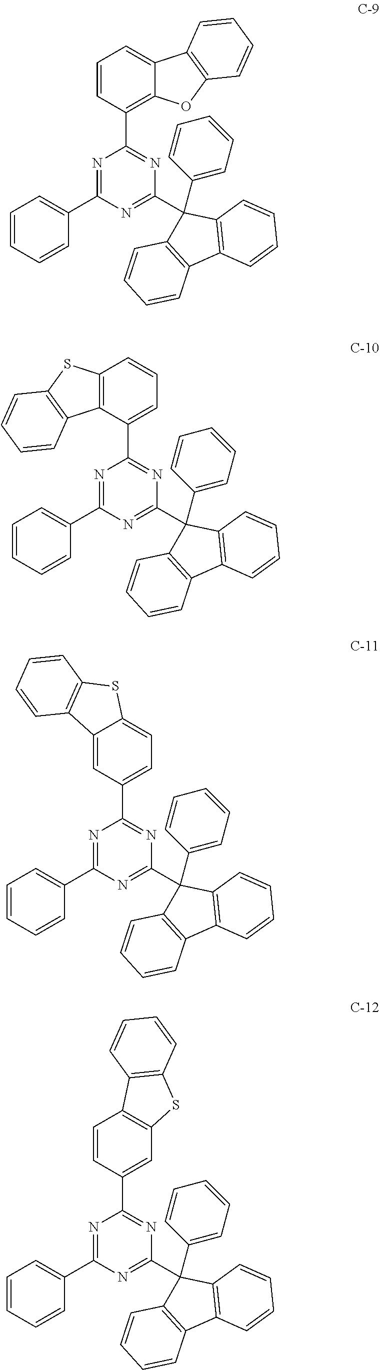 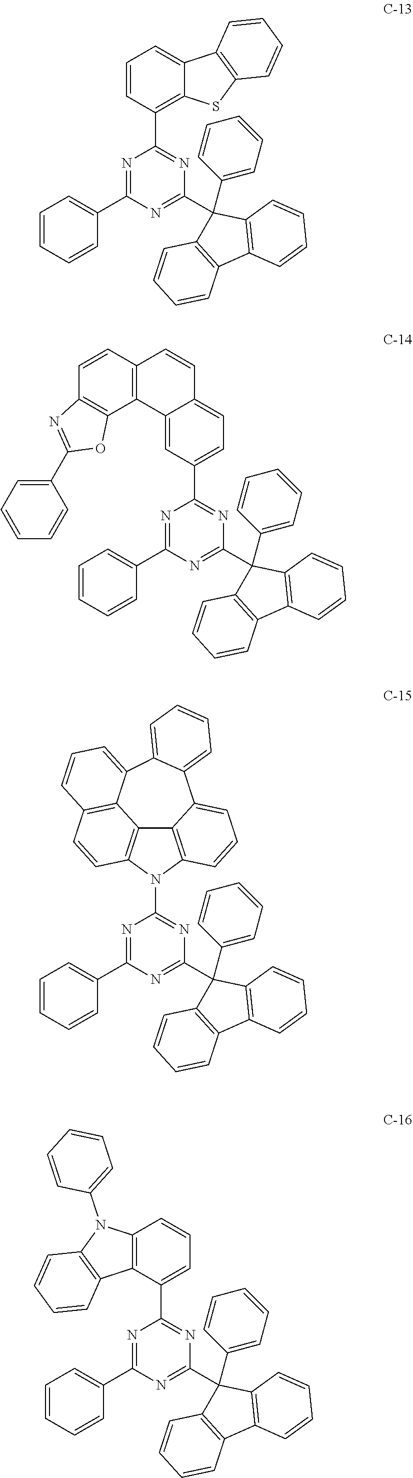 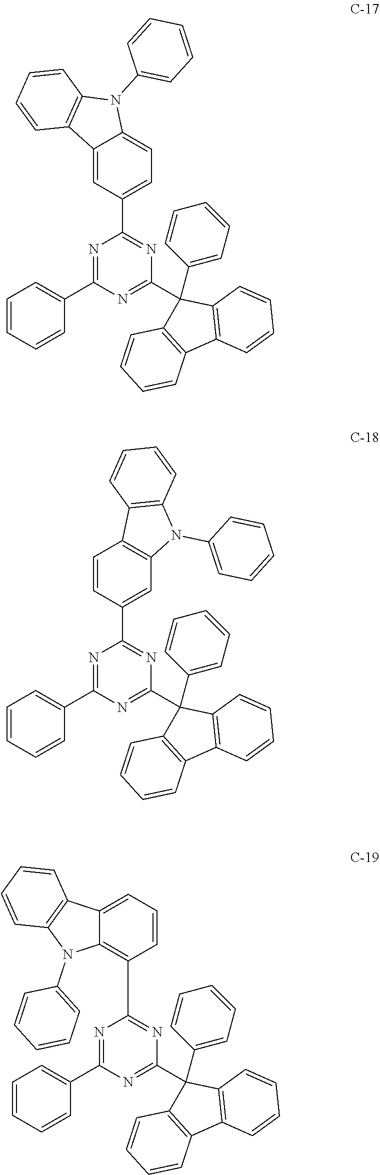 [SMILES: c1ccc(-c2nc(-c3ccc4c(c3)c3ccccc3n4-c3ccccc3)nc(C3(c4ccccc4)c4ccccc4-c4ccccc43)n2)cc1 ; c1ccc(-c2nc(-c3ccc4c5ccccc5n(-c5ccccc5)c4c3)nc(C3(c4ccccc4)c4ccccc4-c4ccccc43)n2)cc1 ; c1ccc(-c2nc(-c3cccc4c5ccccc5n(-c5ccccc5)c34)nc(C3(c4ccccc4)c4ccccc4-c4ccccc43)n2)cc1] 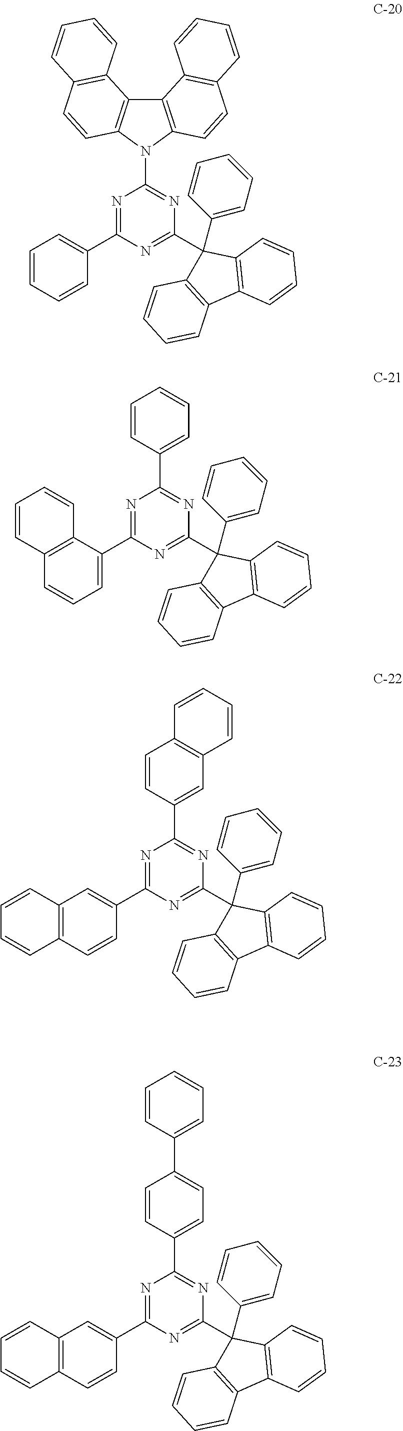 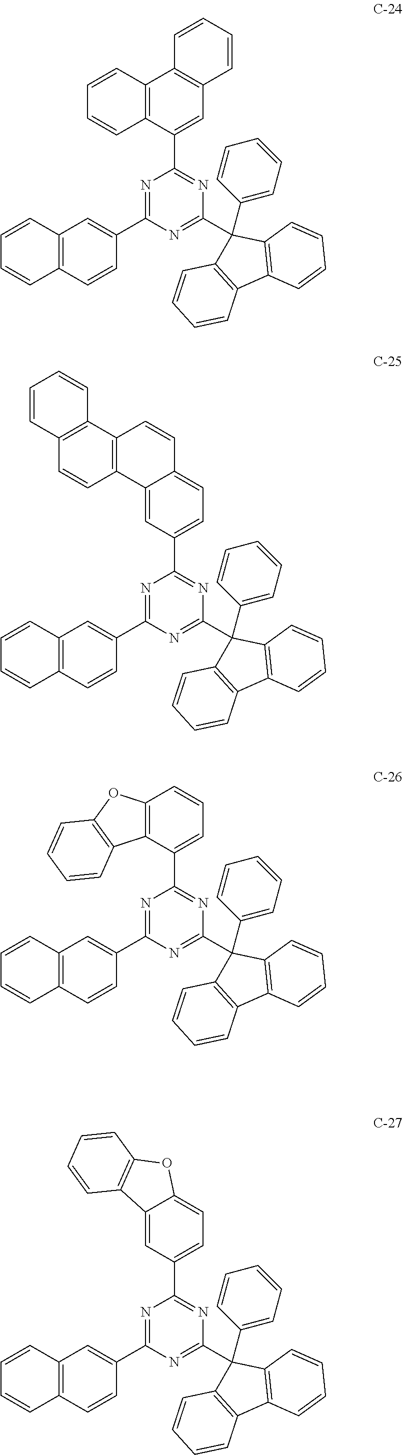 [SMILES: c1ccc(C2(c3nc(-c4ccc5ccccc5c4)nc(-c4cc5ccccc5c5ccccc45)n3)c3ccccc3-c3ccccc32)cc1 ; c1ccc(C2(c3nc(-c4ccc5ccccc5c4)nc(-c4ccc5ccc6c7ccccc7ccc6c5c4)n3)c3ccccc3-c3ccccc32)cc1 ; c1ccc(C2(c3nc(-c4ccc5ccccc5c4)nc(-c4cccc5oc6ccccc6c45)n3)c3ccccc3-c3ccccc32)cc1 ; c1ccc(C2(c3nc(-c4ccc5ccccc5c4)nc(-c4ccc5oc6ccccc6c5c4)n3)c3ccccc3-c3ccccc32)cc1] 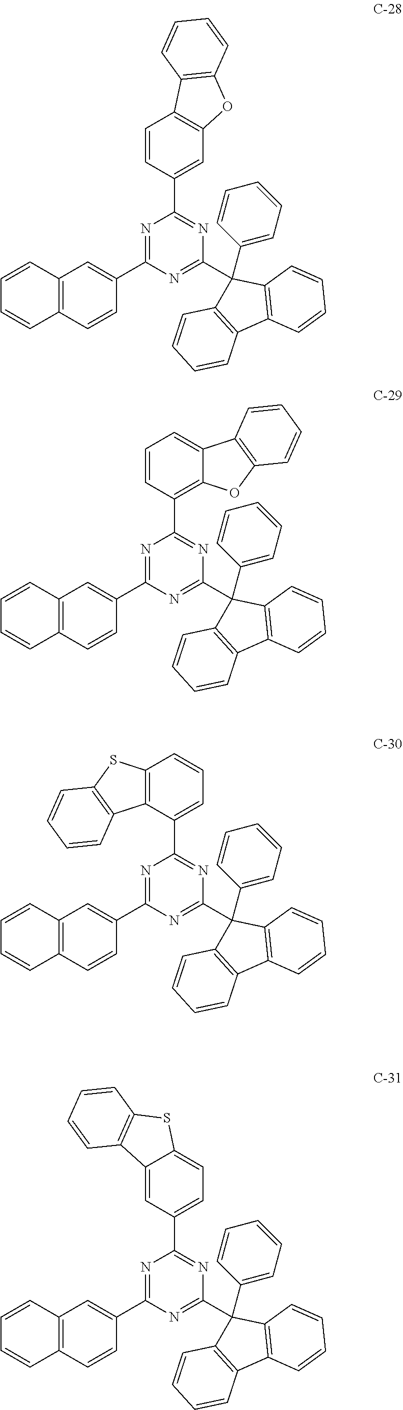 [SMILES: c1ccc(C2(c3nc(-c4ccc5ccccc5c4)nc(-c4ccc5c(c4)oc4ccccc45)n3)c3ccccc3-c3ccccc32)cc1 ; c1ccc(C2(c3nc(-c4ccc5ccccc5c4)nc(-c4cccc5c4oc4ccccc45)n3)c3ccccc3-c3ccccc32)cc1 ; c1ccc(C2(c3nc(-c4ccc5ccccc5c4)nc(-c4cccc5sc6ccccc6c45)n3)c3ccccc3-c3ccccc32)cc1 ; c1ccc(C2(c3nc(-c4ccc5ccccc5c4)nc(-c4ccc5sc6ccccc6c5c4)n3)c3ccccc3-c3ccccc32)cc1] 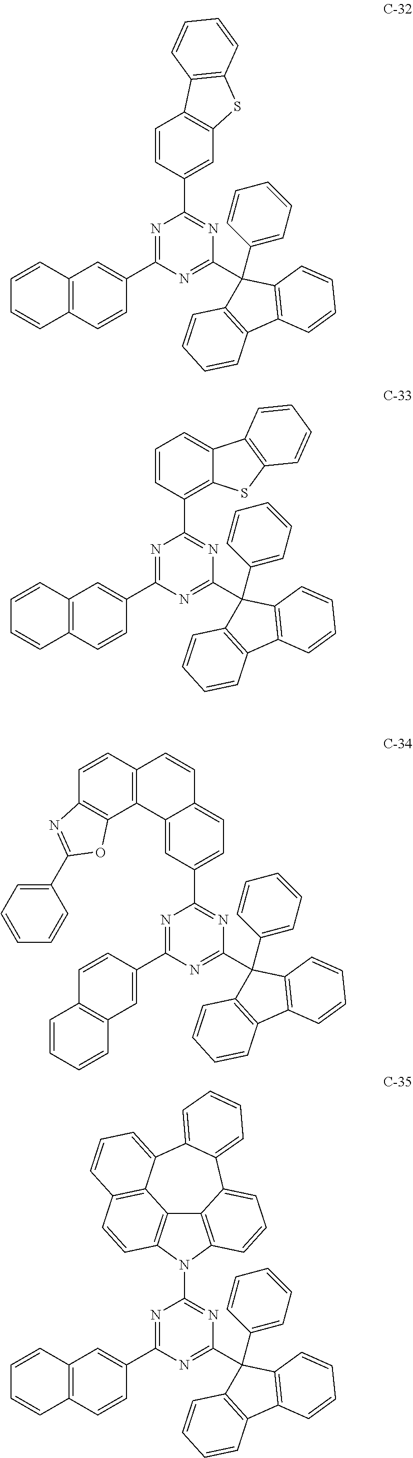 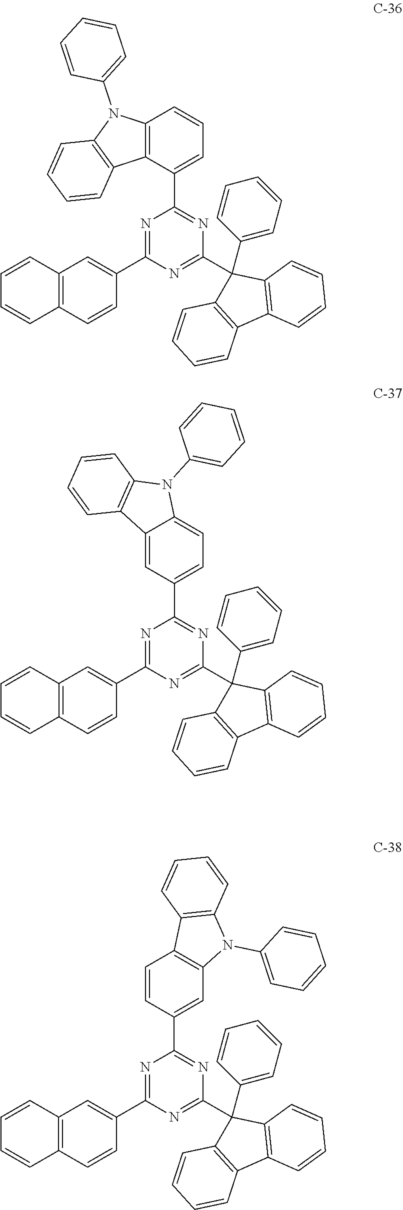 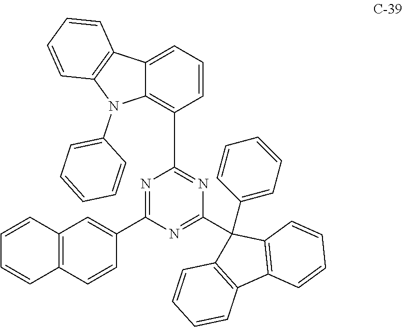 [SMILES: c1ccc(-n2c3ccccc3c3cccc(-c4nc(-c5ccc6ccccc6c5)nc(C5(c6ccccc6)c6ccccc6-c6ccccc65)n4)c32)cc1] 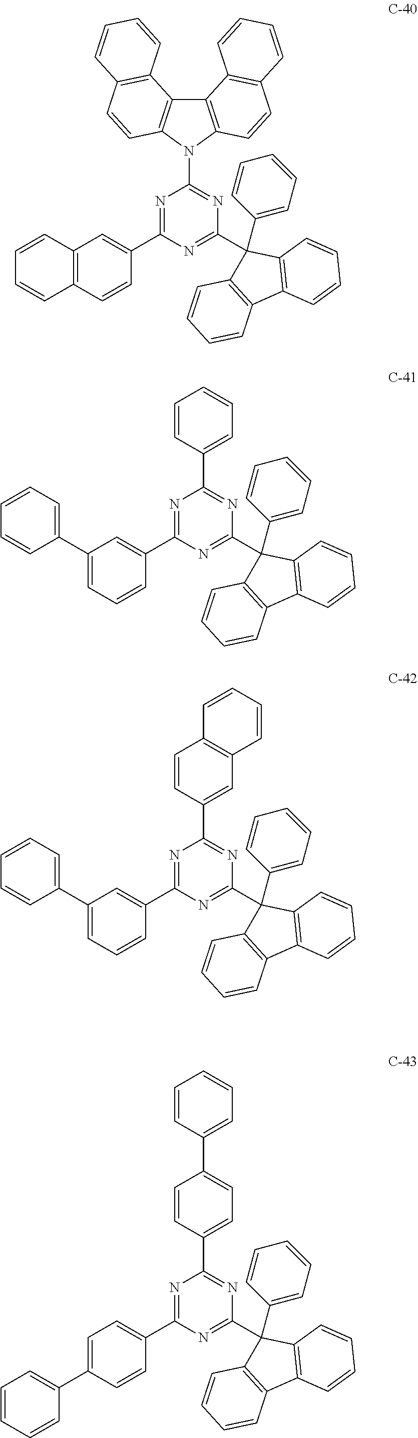 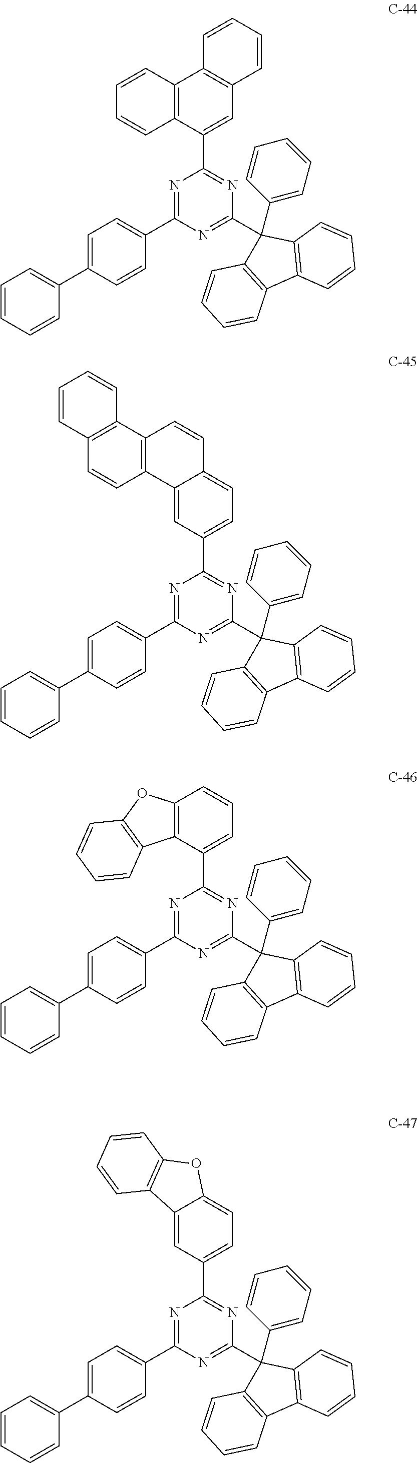 [SMILES: c1ccc(-c2ccc(-c3nc(-c4cc5ccccc5c5ccccc45)nc(C4(c5ccccc5)c5ccccc5-c5ccccc54)n3)cc2)cc1 ; c1ccc(-c2ccc(-c3nc(-c4ccc5ccc6c7ccccc7ccc6c5c4)nc(C4(c5ccccc5)c5ccccc5-c5ccccc54)n3)cc2)cc1 ; c1ccc(-c2ccc(-c3nc(-c4cccc5oc6ccccc6c45)nc(C4(c5ccccc5)c5ccccc5-c5ccccc54)n3)cc2)cc1 ; c1ccc(-c2ccc(-c3nc(-c4ccc5oc6ccccc6c5c4)nc(C4(c5ccccc5)c5ccccc5-c5ccccc54)n3)cc2)cc1] 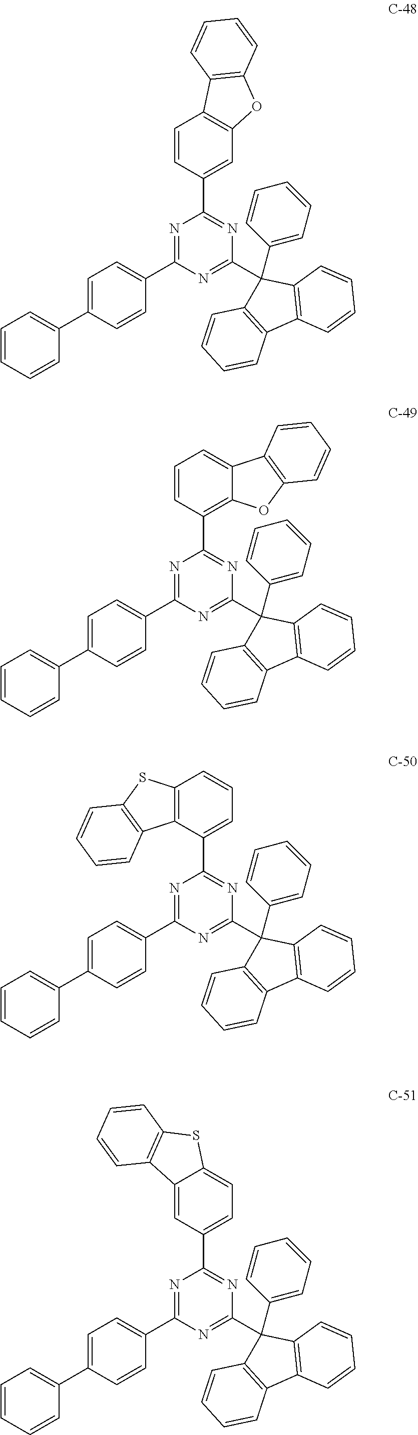 [SMILES: c1ccc(-c2ccc(-c3nc(-c4ccc5c(c4)oc4ccccc45)nc(C4(c5ccccc5)c5ccccc5-c5ccccc54)n3)cc2)cc1 ; c1ccc(-c2ccc(-c3nc(-c4cccc5c4oc4ccccc45)nc(C4(c5ccccc5)c5ccccc5-c5ccccc54)n3)cc2)cc1 ; c1ccc(-c2ccc(-c3nc(-c4cccc5sc6ccccc6c45)nc(C4(c5ccccc5)c5ccccc5-c5ccccc54)n3)cc2)cc1 ; c1ccc(-c2ccc(-c3nc(-c4ccc5sc6ccccc6c5c4)nc(C4(c5ccccc5)c5ccccc5-c5ccccc54)n3)cc2)cc1] 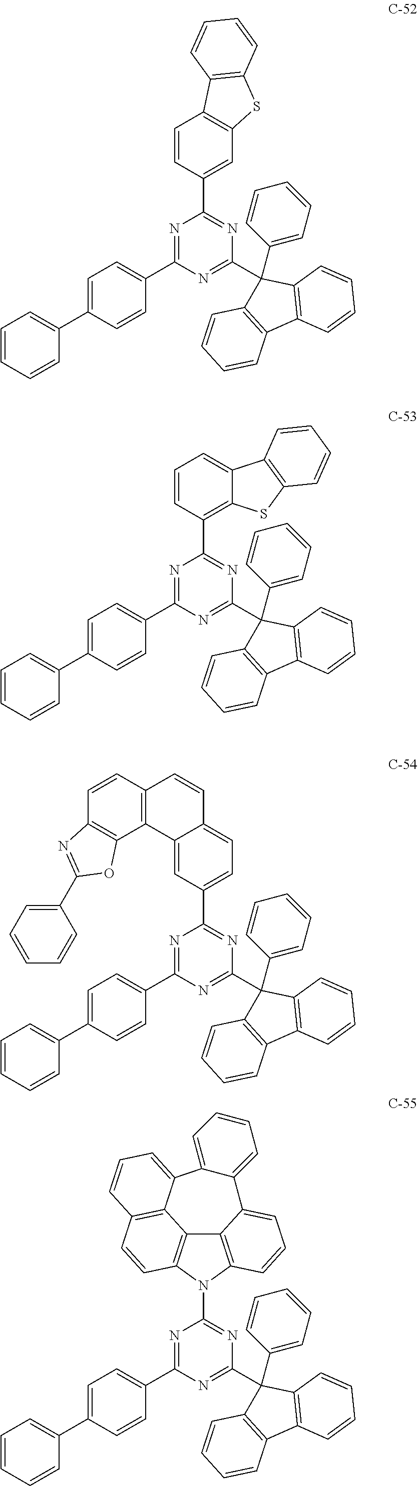 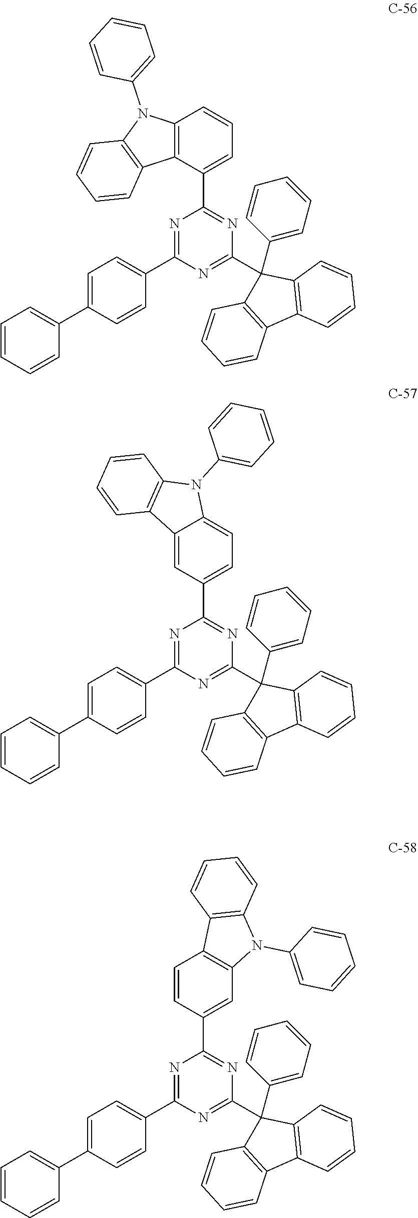 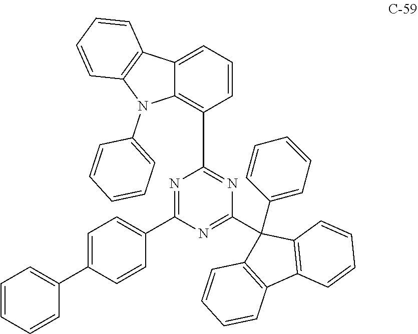 [SMILES: c1ccc(-c2ccc(-c3nc(-c4cccc5c6ccccc6n(-c6ccccc6)c45)nc(C4(c5ccccc5)c5ccccc5-c5ccccc54)n3)cc2)cc1] 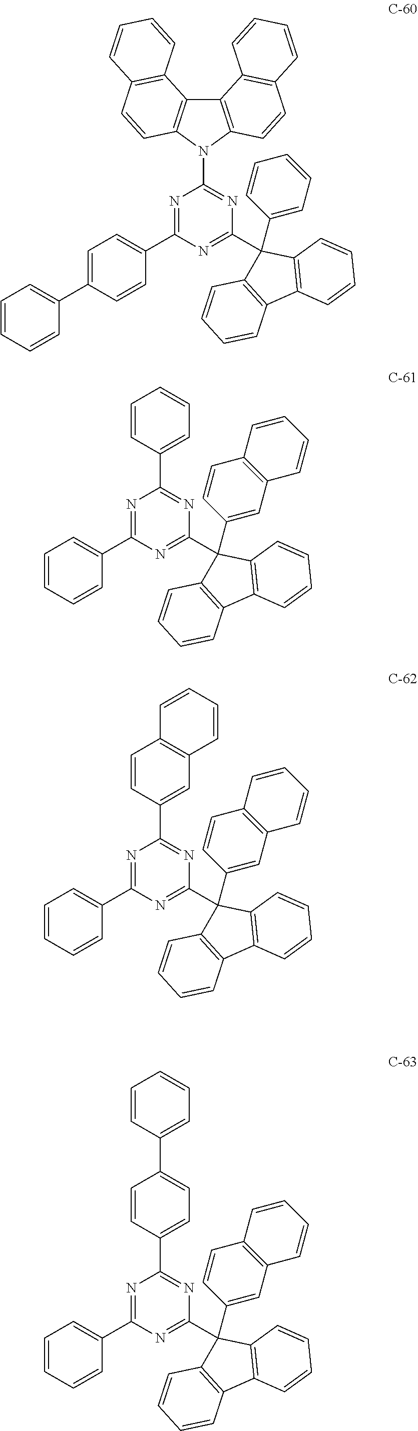 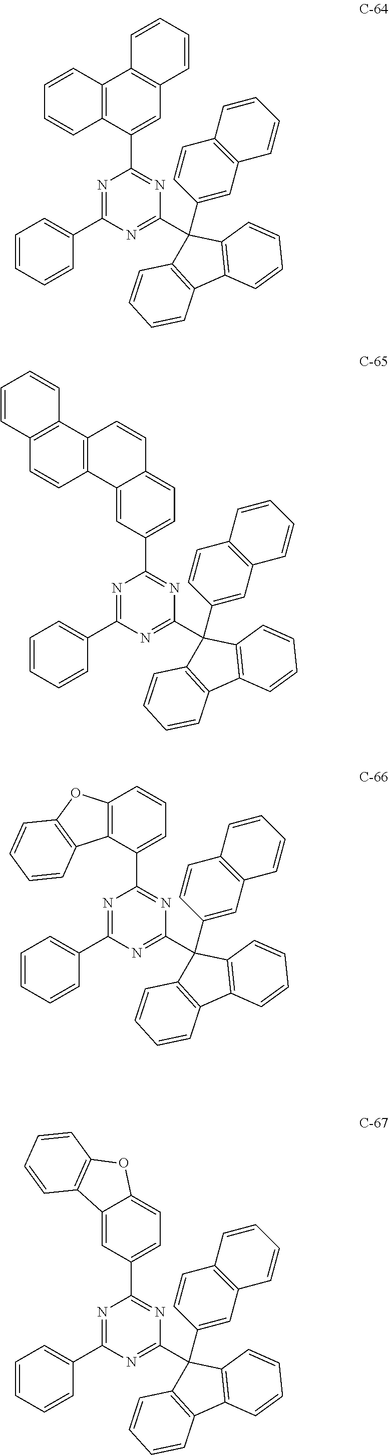 [SMILES: c1ccc(-c2nc(-c3cc4ccccc4c4ccccc34)nc(C3(c4ccc5ccccc5c4)c4ccccc4-c4ccccc43)n2)cc1 ; c1ccc(-c2nc(-c3ccc4ccc5c6ccccc6ccc5c4c3)nc(C3(c4ccc5ccccc5c4)c4ccccc4-c4ccccc43)n2)cc1 ; c1ccc(-c2nc(-c3cccc4oc5ccccc5c34)nc(C3(c4ccc5ccccc5c4)c4ccccc4-c4ccccc43)n2)cc1 ; c1ccc(-c2nc(-c3ccc4oc5ccccc5c4c3)nc(C3(c4ccc5ccccc5c4)c4ccccc4-c4ccccc43)n2)cc1] 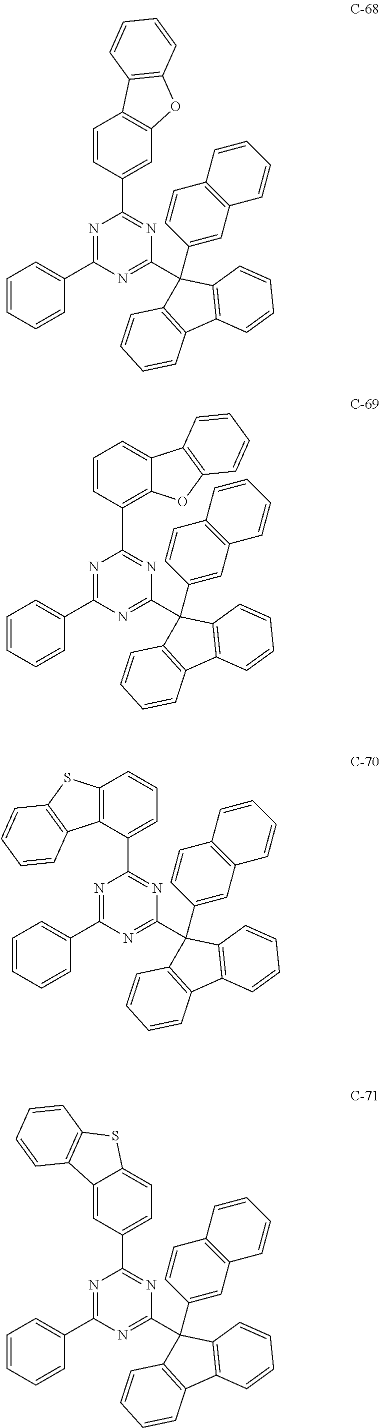 [SMILES: c1ccc(-c2nc(-c3ccc4c(c3)oc3ccccc34)nc(C3(c4ccc5ccccc5c4)c4ccccc4-c4ccccc43)n2)cc1 ; c1ccc(-c2nc(-c3cccc4c3oc3ccccc34)nc(C3(c4ccc5ccccc5c4)c4ccccc4-c4ccccc43)n2)cc1 ; c1ccc(-c2nc(-c3cccc4sc5ccccc5c34)nc(C3(c4ccc5ccccc5c4)c4ccccc4-c4ccccc43)n2)cc1 ; c1ccc(-c2nc(-c3ccc4sc5ccccc5c4c3)nc(C3(c4ccc5ccccc5c4)c4ccccc4-c4ccccc43)n2)cc1] 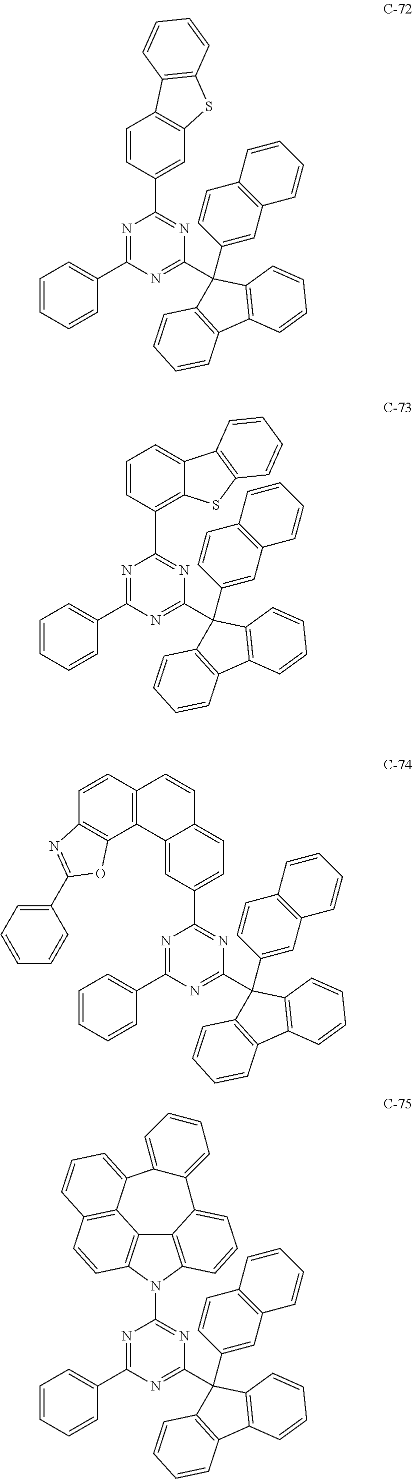 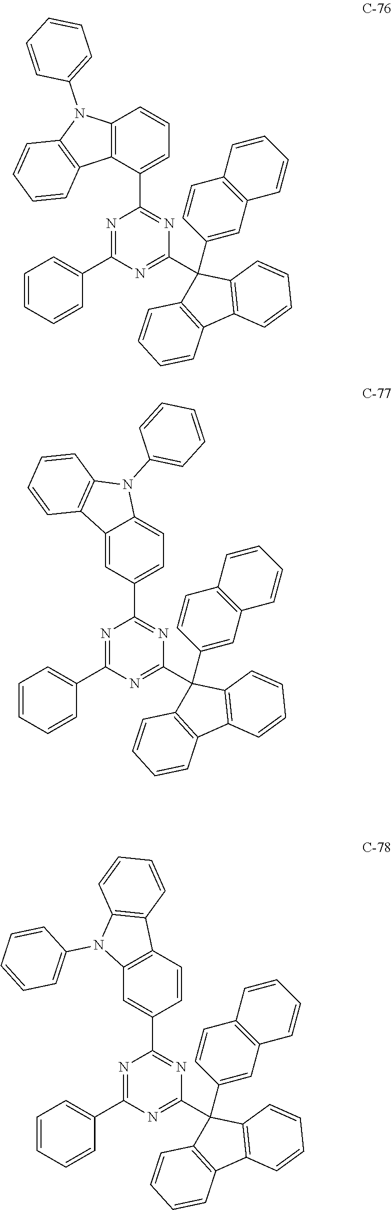 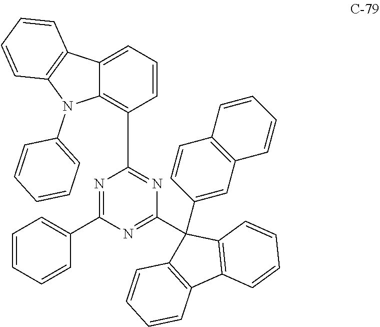 [SMILES: c1ccc(-c2nc(-c3cccc4c5ccccc5n(-c5ccccc5)c34)nc(C3(c4ccc5ccccc5c4)c4ccccc4-c4ccccc43)n2)cc1] 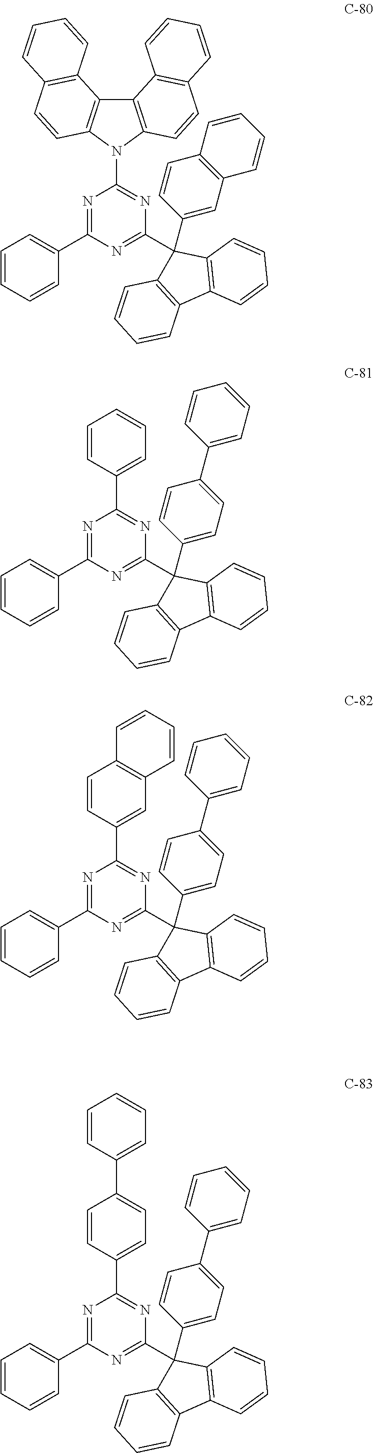 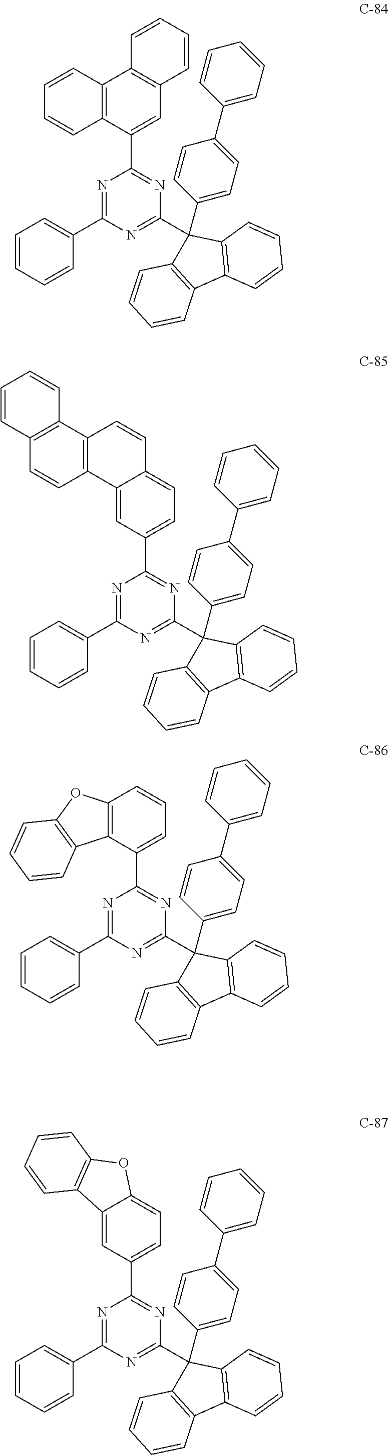 [SMILES: c1ccc(-c2ccc(C3(c4nc(-c5ccccc5)nc(-c5cc6ccccc6c6ccccc56)n4)c4ccccc4-c4ccccc43)cc2)cc1 ; c1ccc(-c2ccc(C3(c4nc(-c5ccccc5)nc(-c5ccc6ccc7c8ccccc8ccc7c6c5)n4)c4ccccc4-c4ccccc43)cc2)cc1 ; c1ccc(-c2ccc(C3(c4nc(-c5ccccc5)nc(-c5cccc6oc7ccccc7c56)n4)c4ccccc4-c4ccccc43)cc2)cc1 ; c1ccc(-c2ccc(C3(c4nc(-c5ccccc5)nc(-c5ccc6oc7ccccc7c6c5)n4)c4ccccc4-c4ccccc43)cc2)cc1] 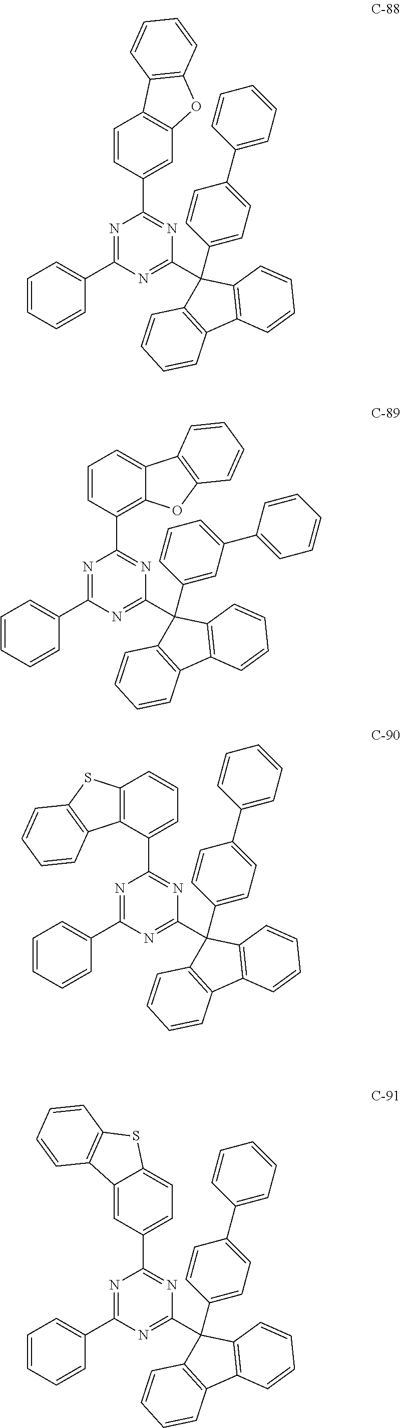 [SMILES: c1ccc(-c2ccc(C3(c4nc(-c5ccccc5)nc(-c5ccc6c(c5)oc5ccccc56)n4)c4ccccc4-c4ccccc43)cc2)cc1 ; c1ccc(-c2cccc(C3(c4nc(-c5ccccc5)nc(-c5cccc6c5oc5ccccc56)n4)c4ccccc4-c4ccccc43)c2)cc1 ; c1ccc(-c2ccc(C3(c4nc(-c5ccccc5)nc(-c5cccc6sc7ccccc7c56)n4)c4ccccc4-c4ccccc43)cc2)cc1 ; c1ccc(-c2ccc(C3(c4nc(-c5ccccc5)nc(-c5ccc6sc7ccccc7c6c5)n4)c4ccccc4-c4ccccc43)cc2)cc1] 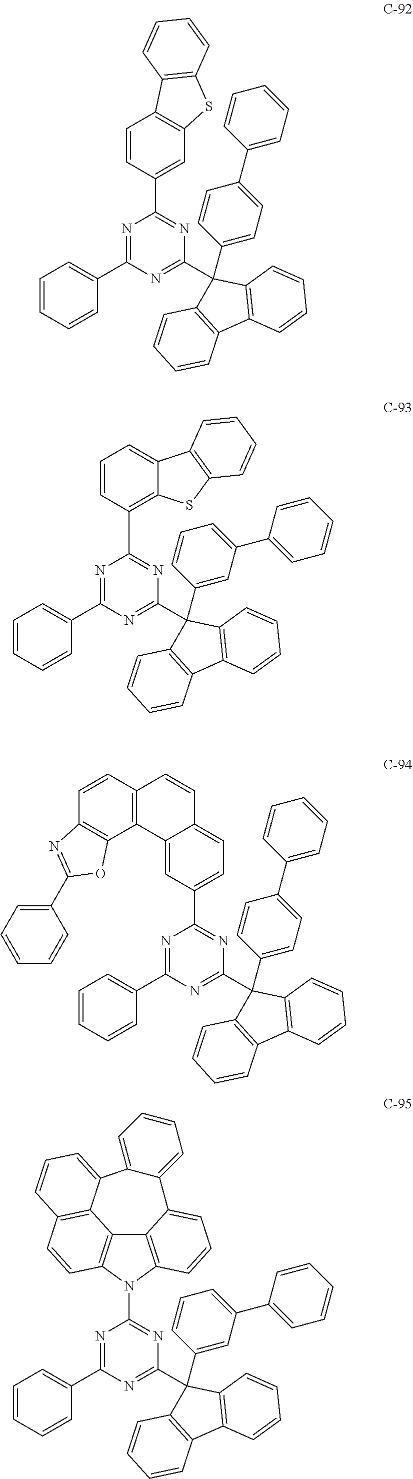 [SMILES: c1ccc(-c2ccc(C3(c4nc(-c5ccccc5)nc(-c5ccc6c(c5)sc5ccccc56)n4)c4ccccc4-c4ccccc43)cc2)cc1 ; c1ccc(-c2cccc(C3(c4nc(-c5ccccc5)nc(-c5cccc6c5sc5ccccc56)n4)c4ccccc4-c4ccccc43)c2)cc1 ; c1ccc(-c2ccc(C3(c4nc(-c5ccccc5)nc(-c5ccc6ccc7ccc8nc(-c9ccccc9)oc8c7c6c5)n4)c4ccccc4-c4ccccc43)cc2)cc1 ; c1ccc(-c2cccc(C3(c4nc(-c5ccccc5)nc(-n5c6cccc7c6c6c8c(cccc8ccc65)-c5ccccc5-7)n4)c4ccccc4-c4ccccc43)c2)cc1] 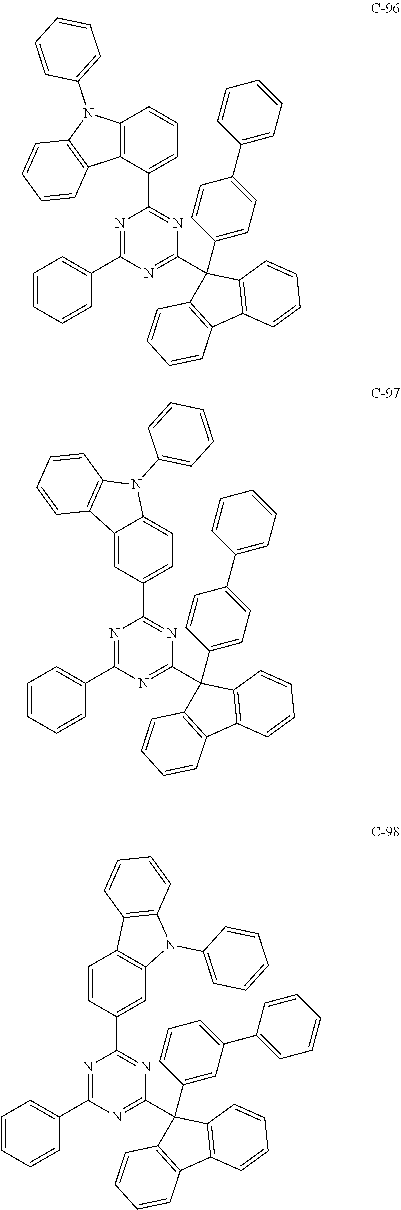 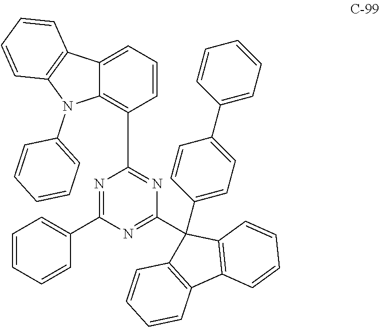 [SMILES: c1ccc(-c2ccc(C3(c4nc(-c5ccccc5)nc(-c5cccc6c7ccccc7n(-c7ccccc7)c56)n4)c4ccccc4-c4ccccc43)cc2)cc1] 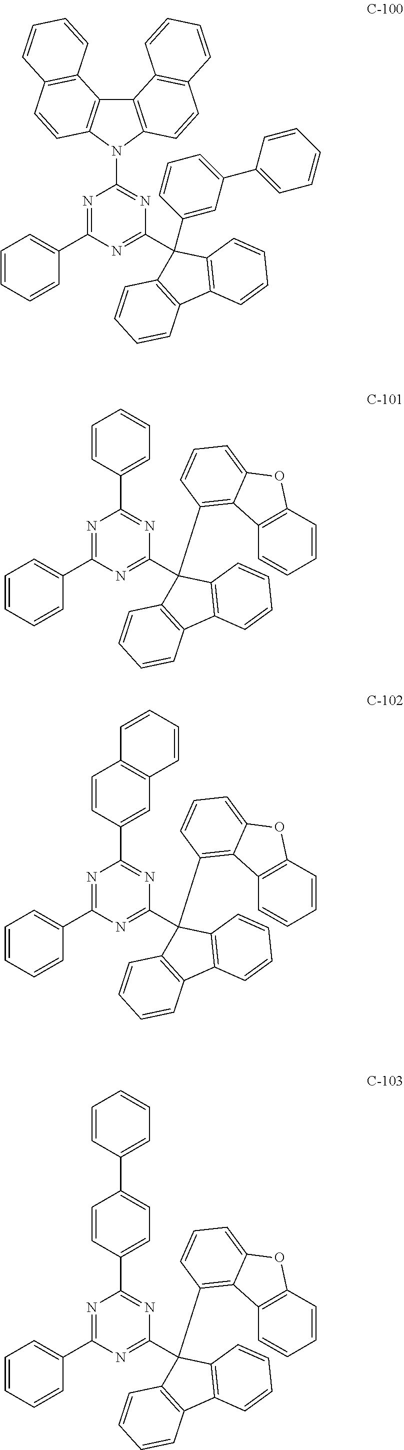 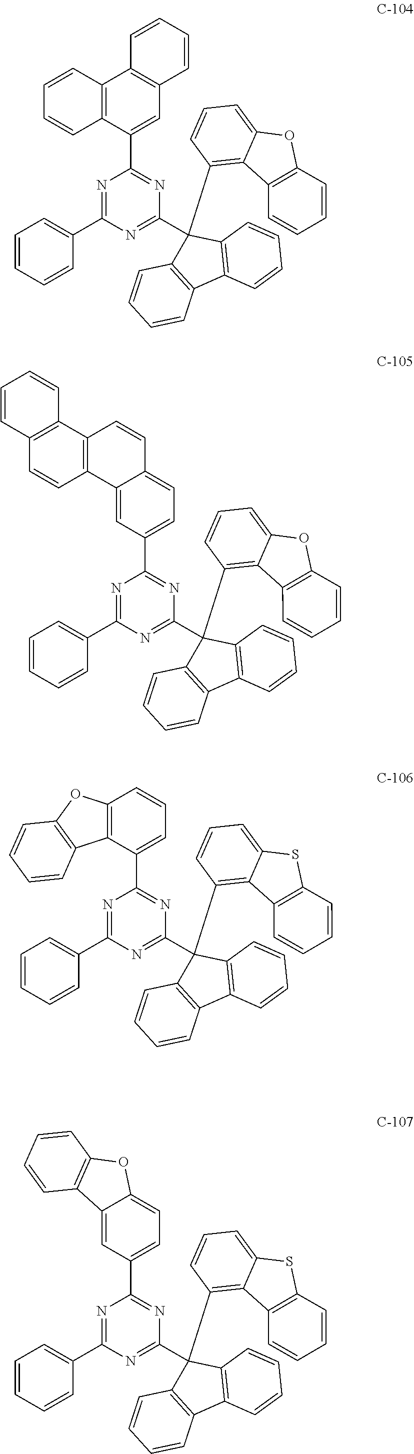 [SMILES: c1ccc(-c2nc(-c3cc4ccccc4c4ccccc34)nc(C3(c4cccc5oc6ccccc6c45)c4ccccc4-c4ccccc43)n2)cc1 ; c1ccc(-c2nc(-c3ccc4ccc5c6ccccc6ccc5c4c3)nc(C3(c4cccc5oc6ccccc6c45)c4ccccc4-c4ccccc43)n2)cc1 ; c1ccc(-c2nc(-c3cccc4oc5ccccc5c34)nc(C3(c4cccc5sc6ccccc6c45)c4ccccc4-c4ccccc43)n2)cc1 ; c1ccc(-c2nc(-c3ccc4oc5ccccc5c4c3)nc(C3(c4cccc5sc6ccccc6c45)c4ccccc4-c4ccccc43)n2)cc1] 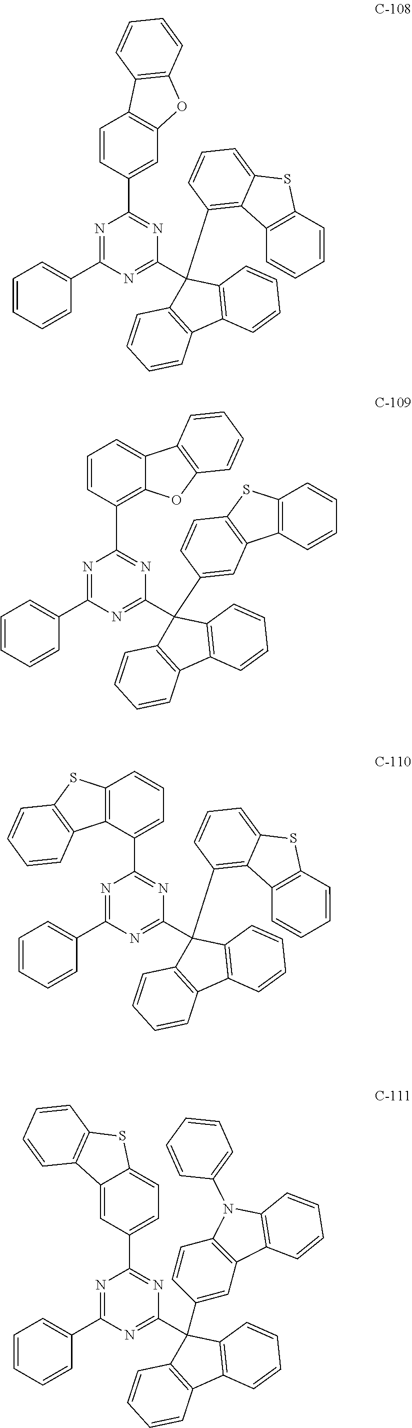 [SMILES: c1ccc(-c2nc(-c3ccc4c(c3)oc3ccccc34)nc(C3(c4cccc5sc6ccccc6c45)c4ccccc4-c4ccccc43)n2)cc1 ; c1ccc(-c2nc(-c3cccc4c3oc3ccccc34)nc(C3(c4ccc5sc6ccccc6c5c4)c4ccccc4-c4ccccc43)n2)cc1 ; c1ccc(-c2nc(-c3cccc4sc5ccccc5c34)nc(C3(c4cccc5sc6ccccc6c45)c4ccccc4-c4ccccc43)n2)cc1 ; c1ccc(-c2nc(-c3ccc4sc5ccccc5c4c3)nc(C3(c4ccc5c(c4)c4ccccc4n5-c4ccccc4)c4ccccc4-c4ccccc43)n2)cc1] 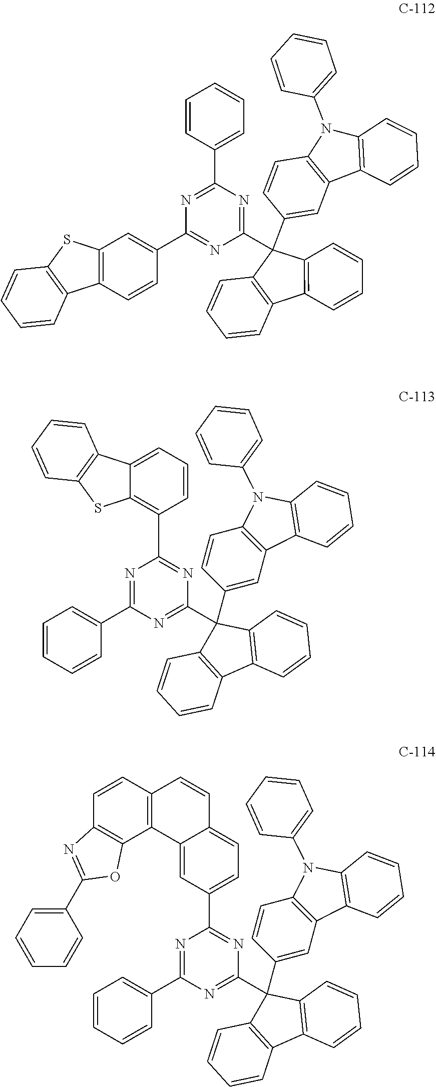 [SMILES: c1ccc(-c2nc(-c3ccc4c(c3)sc3ccccc34)nc(C3(c4ccc5c(c4)c4ccccc4n5-c4ccccc4)c4ccccc4-c4ccccc43)n2)cc1 ; c1ccc(-c2nc(-c3cccc4c3sc3ccccc34)nc(C3(c4ccc5c(c4)c4ccccc4n5-c4ccccc4)c4ccccc4-c4ccccc43)n2)cc1 ; c1ccc(-c2nc(-c3ccc4ccc5ccc6nc(-c7ccccc7)oc6c5c4c3)nc(C3(c4ccc5c(c4)c4ccccc4n5-c4ccccc4)c4ccccc4-c4ccccc43)n2)cc1] 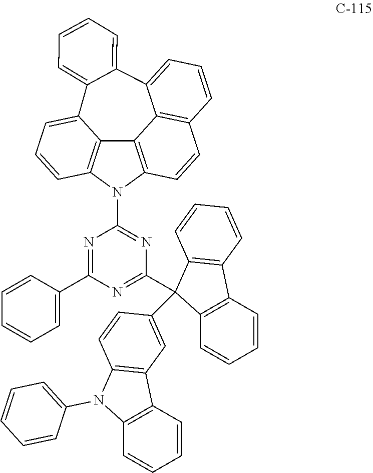 [SMILES: c1ccc(-c2nc(-n3c4cccc5c4c4c6c(cccc6ccc43)-c3ccccc3-5)nc(C3(c4ccc5c(c4)c4ccccc4n5-c4ccccc4)c4ccccc4-c4ccccc43)n2)cc1] 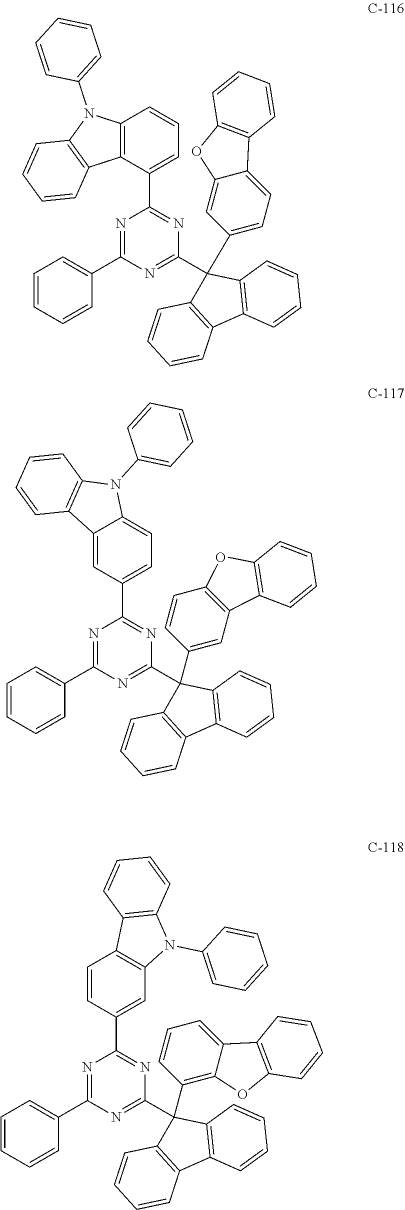 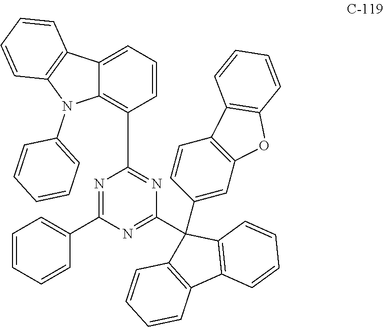 [SMILES: c1ccc(-c2nc(-c3cccc4c5ccccc5n(-c5ccccc5)c34)nc(C3(c4ccc5c(c4)oc4ccccc45)c4ccccc4-c4ccccc43)n2)cc1] 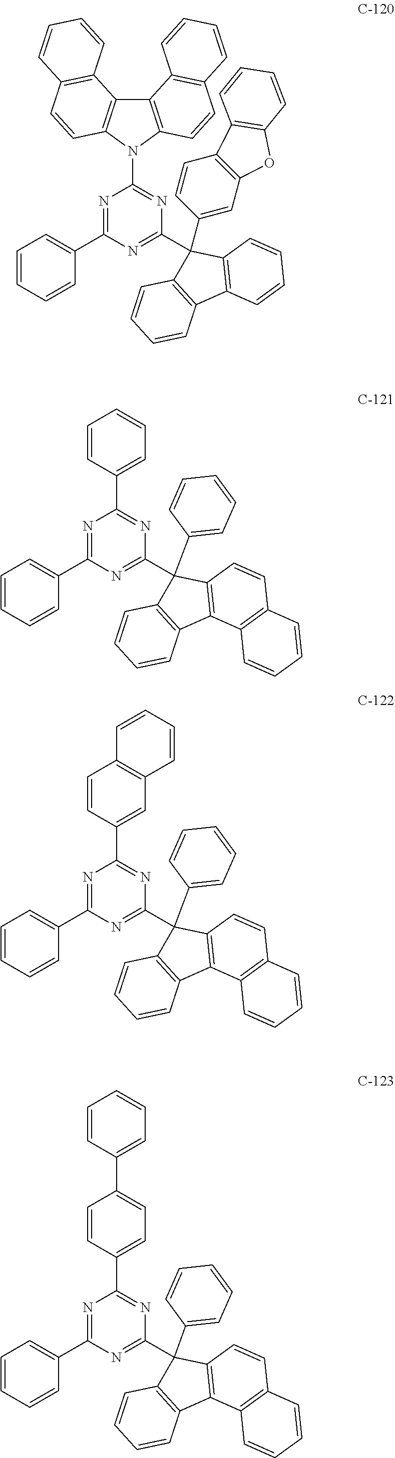 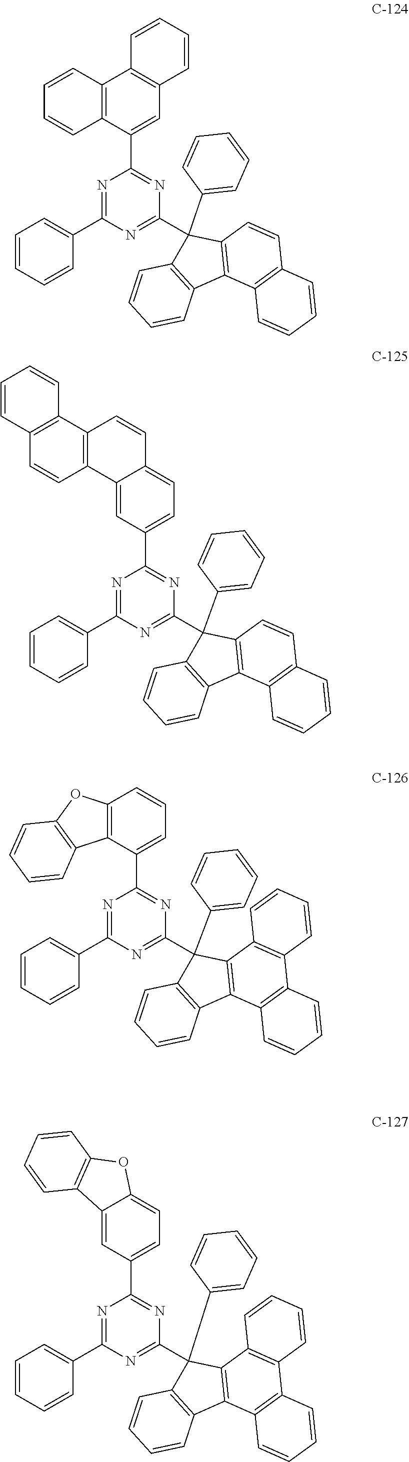 [SMILES: c1ccc(-c2nc(-c3cc4ccccc4c4ccccc34)nc(C3(c4ccccc4)c4ccccc4-c4c3ccc3ccccc43)n2)cc1 ; c1ccc(-c2nc(-c3ccc4ccc5c6ccccc6ccc5c4c3)nc(C3(c4ccccc4)c4ccccc4-c4c3ccc3ccccc43)n2)cc1 ; c1ccc(-c2nc(-c3cccc4oc5ccccc5c34)nc(C3(c4ccccc4)c4ccccc4-c4c3c3ccccc3c3ccccc43)n2)cc1 ; c1ccc(-c2nc(-c3ccc4oc5ccccc5c4c3)nc(C3(c4ccccc4)c4ccccc4-c4c3c3ccccc3c3ccccc43)n2)cc1] 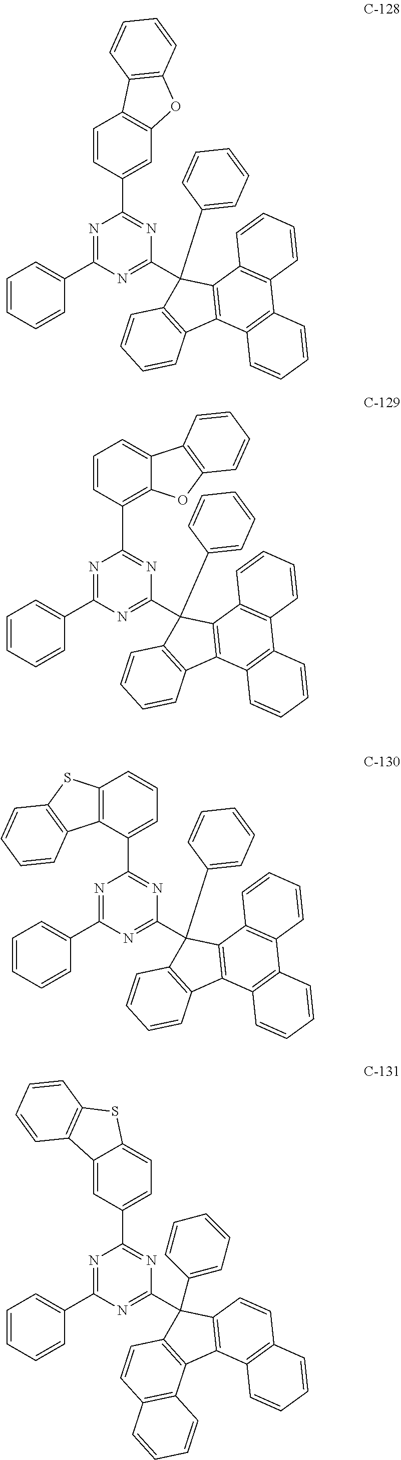 [SMILES: c1ccc(-c2nc(-c3ccc4c(c3)oc3ccccc34)nc(C3(c4ccccc4)c4ccccc4-c4c3c3ccccc3c3ccccc43)n2)cc1 ; c1ccc(-c2nc(-c3cccc4c3oc3ccccc34)nc(C3(c4ccccc4)c4ccccc4-c4c3c3ccccc3c3ccccc43)n2)cc1 ; c1ccc(-c2nc(-c3cccc4sc5ccccc5c34)nc(C3(c4ccccc4)c4ccccc4-c4c3c3ccccc3c3ccccc43)n2)cc1 ; c1ccc(-c2nc(-c3ccc4sc5ccccc5c4c3)nc(C3(c4ccccc4)c4ccc5ccccc5c4-c4c3ccc3ccccc43)n2)cc1] 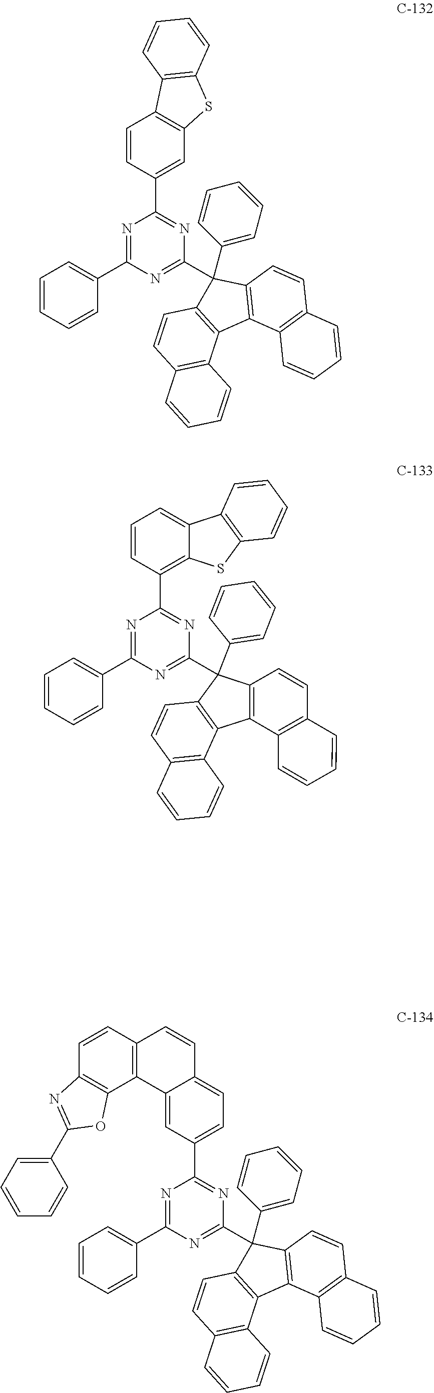 [SMILES: c1ccc(-c2nc(-c3ccc4c(c3)sc3ccccc34)nc(C3(c4ccccc4)c4ccc5ccccc5c4-c4c3ccc3ccccc43)n2)cc1 ; c1ccc(-c2nc(-c3cccc4c3sc3ccccc34)nc(C3(c4ccccc4)c4ccc5ccccc5c4-c4c3ccc3ccccc43)n2)cc1 ; c1ccc(-c2nc(-c3ccc4ccc5ccc6nc(-c7ccccc7)oc6c5c4c3)nc(C3(c4ccccc4)c4ccc5ccccc5c4-c4c3ccc3ccccc43)n2)cc1] 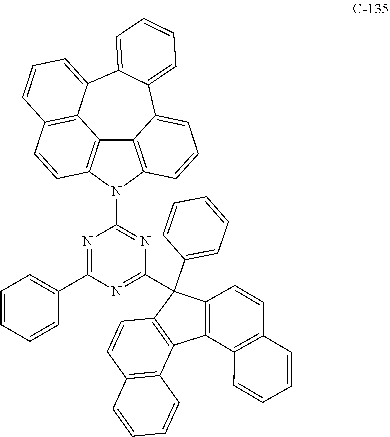 [SMILES: c1ccc(-c2nc(-n3c4cccc5c4c4c6c(cccc6ccc43)-c3ccccc3-5)nc(C3(c4ccccc4)c4ccc5ccccc5c4-c4c3ccc3ccccc43)n2)cc1] 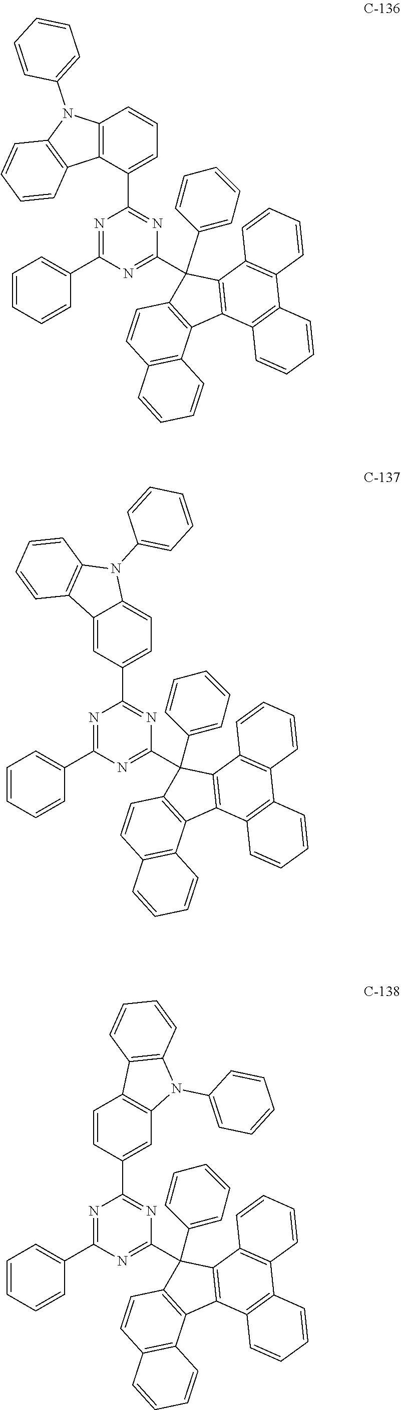 [SMILES: c1ccc(-c2nc(-c3cccc4c3c3ccccc3n4-c3ccccc3)nc(C3(c4ccccc4)c4ccc5ccccc5c4-c4c3c3ccccc3c3ccccc43)n2)cc1 ; c1ccc(-c2nc(-c3ccc4c(c3)c3ccccc3n4-c3ccccc3)nc(C3(c4ccccc4)c4ccc5ccccc5c4-c4c3c3ccccc3c3ccccc43)n2)cc1 ; c1ccc(-c2nc(-c3ccc4c5ccccc5n(-c5ccccc5)c4c3)nc(C3(c4ccccc4)c4ccc5ccccc5c4-c4c3c3ccccc3c3ccccc43)n2)cc1] 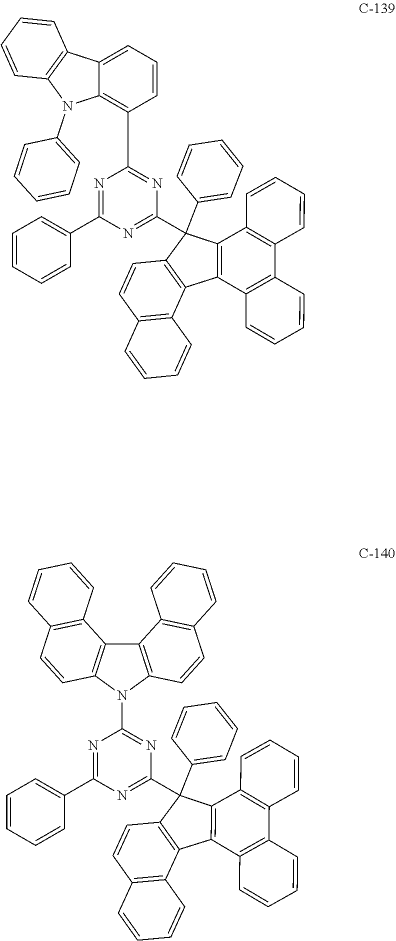 [SMILES: c1ccc(-c2nc(-c3cccc4c5ccccc5n(-c5ccccc5)c34)nc(C3(c4ccccc4)c4ccc5ccccc5c4-c4c3c3ccccc3c3ccccc43)n2)cc1 ; c1ccc(-c2nc(-n3c4ccc5ccccc5c4c4c5ccccc5ccc43)nc(C3(c4ccccc4)c4ccc5ccccc5c4-c4c3c3ccccc3c3ccccc43)n2)cc1] 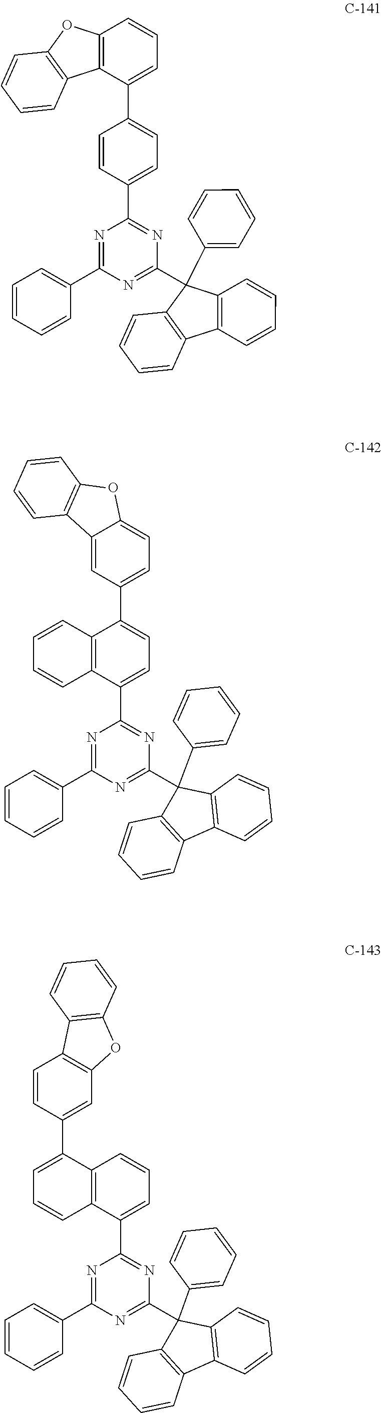 [SMILES: c1ccc(-c2nc(-c3ccc(-c4cccc5oc6ccccc6c45)cc3)nc(C3(c4ccccc4)c4ccccc4-c4ccccc43)n2)cc1 ; c1ccc(-c2nc(-c3ccc(-c4ccc5oc6ccccc6c5c4)c4ccccc34)nc(C3(c4ccccc4)c4ccccc4-c4ccccc43)n2)cc1 ; c1ccc(-c2nc(-c3cccc4c(-c5ccc6c(c5)oc5ccccc56)cccc34)nc(C3(c4ccccc4)c4ccccc4-c4ccccc43)n2)cc1] 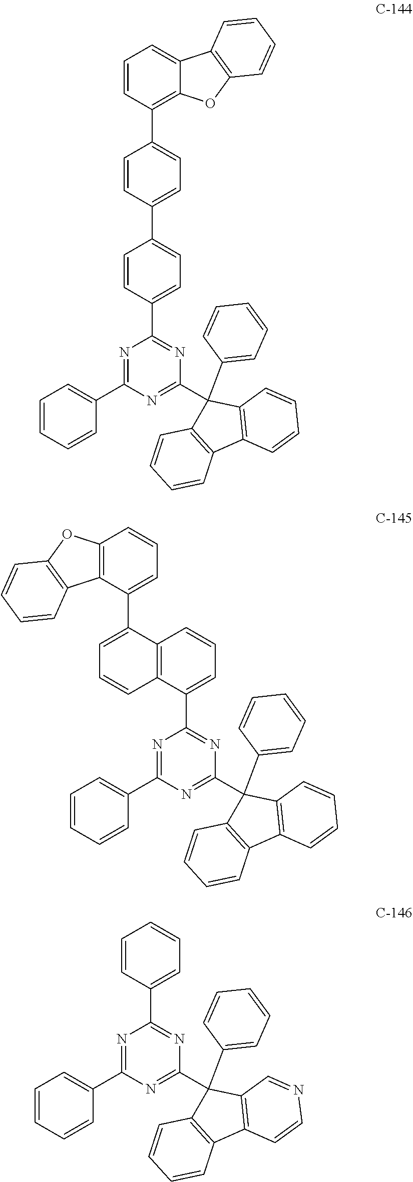 [SMILES: c1ccc(-c2nc(-c3ccc(-c4ccc(-c5cccc6c5oc5ccccc56)cc4)cc3)nc(C3(c4ccccc4)c4ccccc4-c4ccccc43)n2)cc1 ; c1ccc(-c2nc(-c3cccc4c(-c5cccc6oc7ccccc7c56)cccc34)nc(C3(c4ccccc4)c4ccccc4-c4ccccc43)n2)cc1 ; c1ccc(-c2nc(-c3ccccc3)nc(C3(c4ccccc4)c4ccccc4-c4ccncc43)n2)cc1] 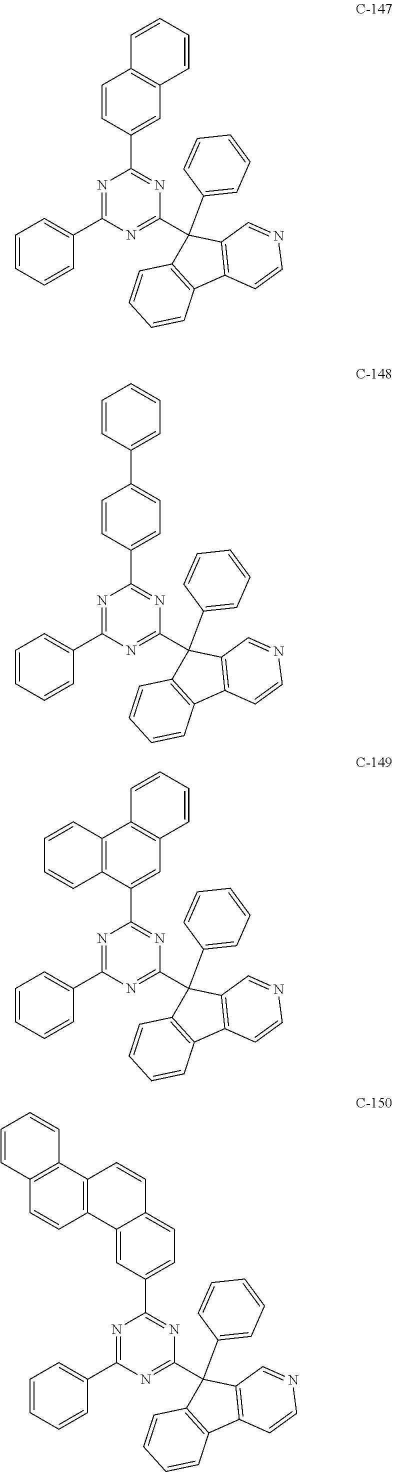 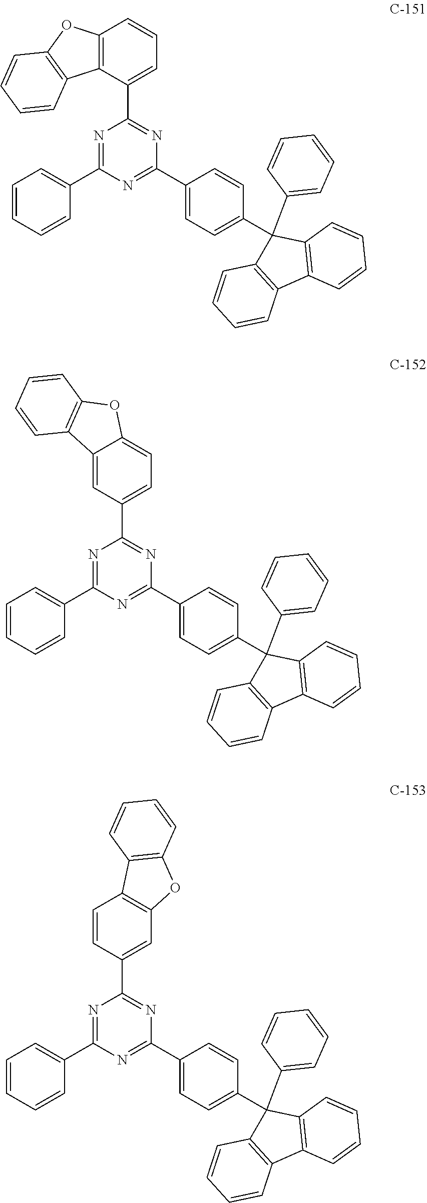 [SMILES: c1ccc(-c2nc(-c3ccc(C4(c5ccccc5)c5ccccc5-c5ccccc54)cc3)nc(-c3cccc4oc5ccccc5c34)n2)cc1 ; c1ccc(-c2nc(-c3ccc(C4(c5ccccc5)c5ccccc5-c5ccccc54)cc3)nc(-c3ccc4oc5ccccc5c4c3)n2)cc1 ; c1ccc(-c2nc(-c3ccc(C4(c5ccccc5)c5ccccc5-c5ccccc54)cc3)nc(-c3ccc4c(c3)oc3ccccc34)n2)cc1] 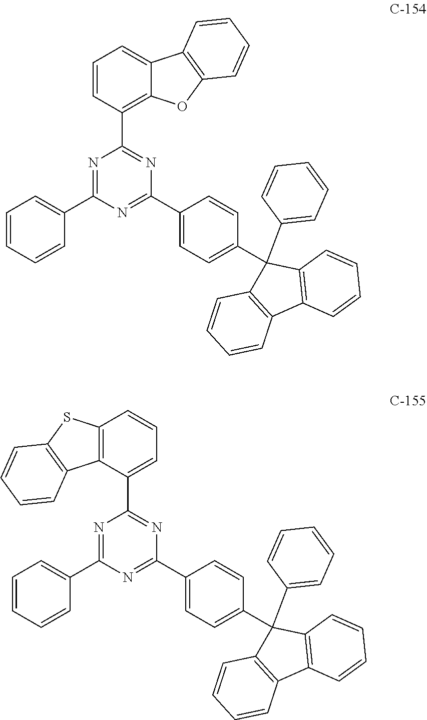 [SMILES: c1ccc(-c2nc(-c3ccc(C4(c5ccccc5)c5ccccc5-c5ccccc54)cc3)nc(-c3cccc4c3oc3ccccc34)n2)cc1 ; c1ccc(-c2nc(-c3ccc(C4(c5ccccc5)c5ccccc5-c5ccccc54)cc3)nc(-c3cccc4sc5ccccc5c34)n2)cc1] 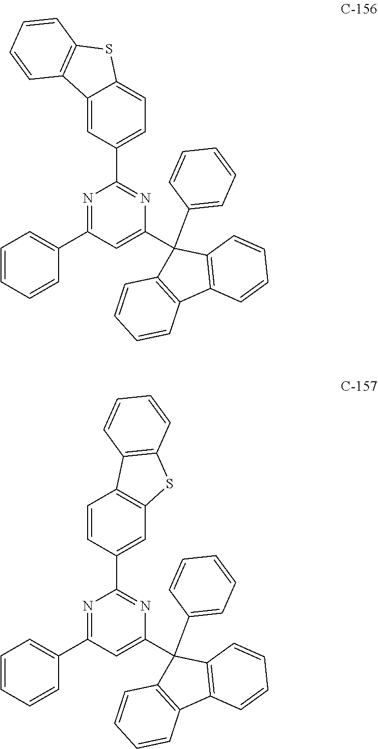 [SMILES: c1ccc(-c2cc(C3(c4ccccc4)c4ccccc4-c4ccccc43)nc(-c3ccc4sc5ccccc5c4c3)n2)cc1 ; c1ccc(-c2cc(C3(c4ccccc4)c4ccccc4-c4ccccc43)nc(-c3ccc4c(c3)sc3ccccc34)n2)cc1] 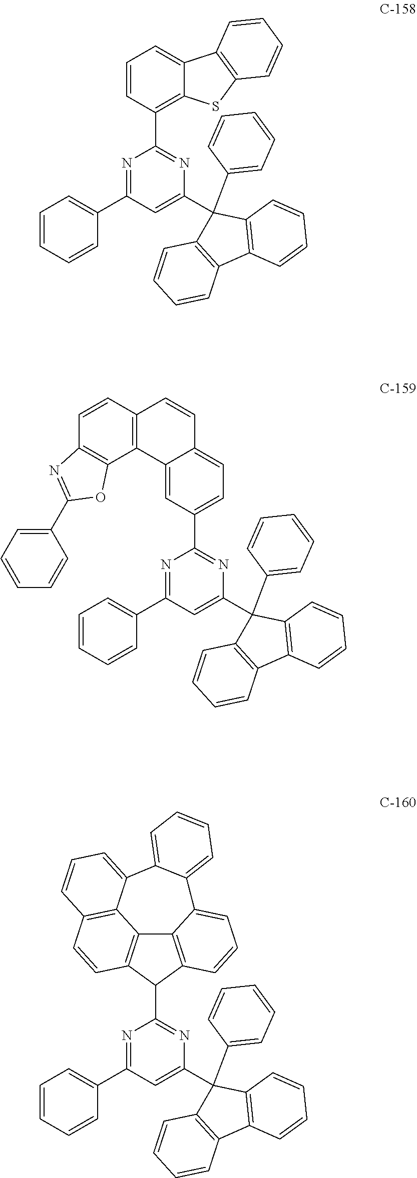 [SMILES: c1ccc(-c2cc(C3(c4ccccc4)c4ccccc4-c4ccccc43)nc(-c3cccc4c3sc3ccccc34)n2)cc1 ; c1ccc(-c2cc(C3(c4ccccc4)c4ccccc4-c4ccccc43)nc(-c3ccc4ccc5ccc6nc(-c7ccccc7)oc6c5c4c3)n2)cc1 ; c1ccc(-c2cc(C3(c4ccccc4)c4ccccc4-c4ccccc43)nc(C3c4cccc5c4-c4c3ccc3cccc(c43)-c3ccccc3-5)n2)cc1] 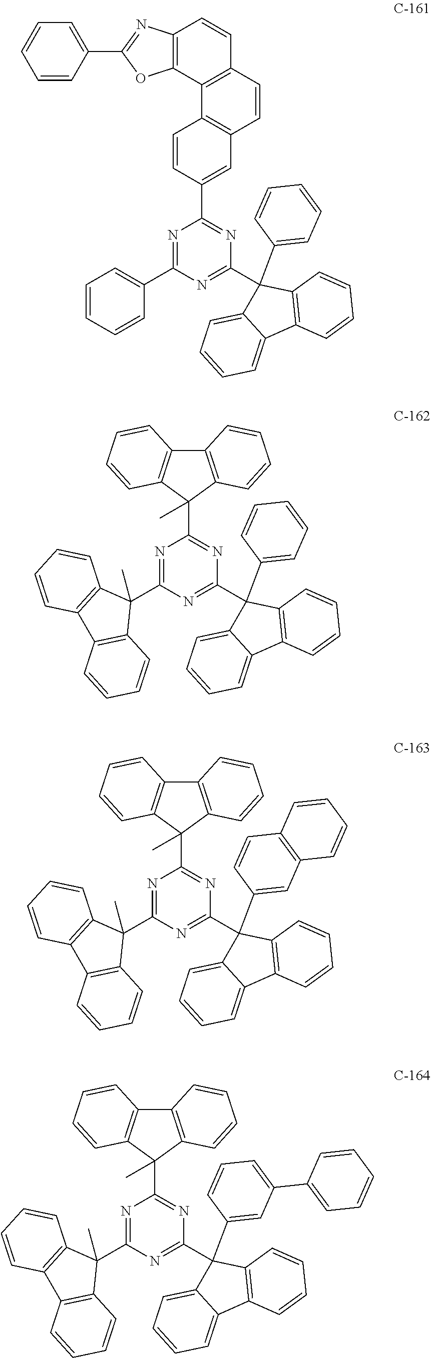 [SMILES: c1ccc(-c2nc(-c3ccc4c(ccc5ccc6nc(-c7ccccc7)oc6c54)c3)nc(C3(c4ccccc4)c4ccccc4-c4ccccc43)n2)cc1 ; CC1(c2nc(C3(C)c4ccccc4-c4ccccc43)nc(C3(c4ccccc4)c4ccccc4-c4ccccc43)n2)c2ccccc2-c2ccccc21 ; CC1(c2nc(C3(C)c4ccccc4-c4ccccc43)nc(C3(c4ccc5ccccc5c4)c4ccccc4-c4ccccc43)n2)c2ccccc2-c2ccccc21 ; CC1(c2nc(C3(C)c4ccccc4-c4ccccc43)nc(C3(c4cccc(-c5ccccc5)c4)c4ccccc4-c4ccccc43)n2)c2ccccc2-c2ccccc21] 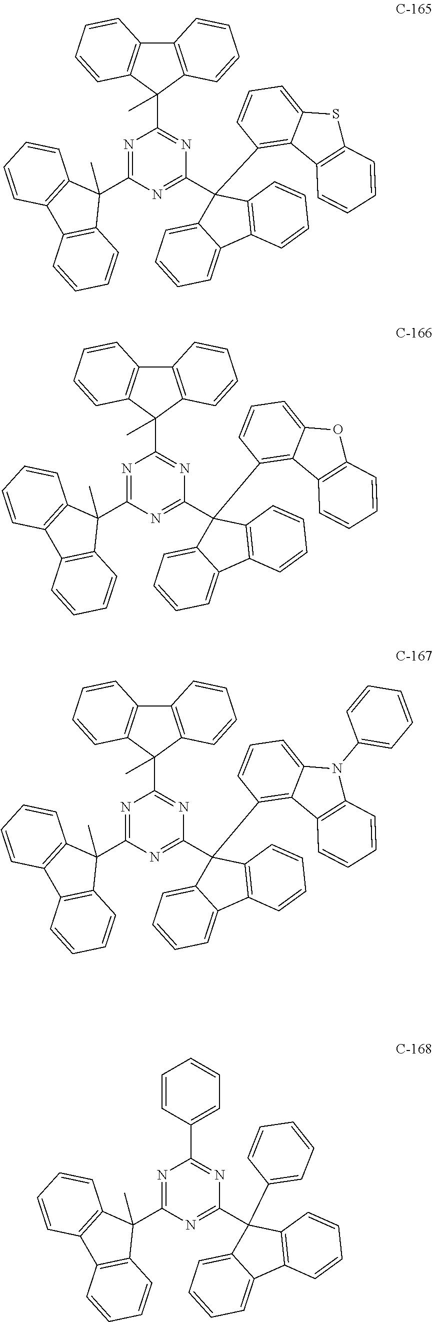 [SMILES: CC1(c2nc(C3(C)c4ccccc4-c4ccccc43)nc(C3(c4cccc5sc6ccccc6c45)c4ccccc4-c4ccccc43)n2)c2ccccc2-c2ccccc21 ; CC1(c2nc(C3(C)c4ccccc4-c4ccccc43)nc(C3(c4cccc5oc6ccccc6c45)c4ccccc4-c4ccccc43)n2)c2ccccc2-c2ccccc21 ; CC1(c2nc(C3(C)c4ccccc4-c4ccccc43)nc(C3(c4cccc5c4c4ccccc4n5-c4ccccc4)c4ccccc4-c4ccccc43)n2)c2ccccc2-c2ccccc21 ; CC1(c2nc(-c3ccccc3)nc(C3(c4ccccc4)c4ccccc4-c4ccccc43)n2)c2ccccc2-c2ccccc21] 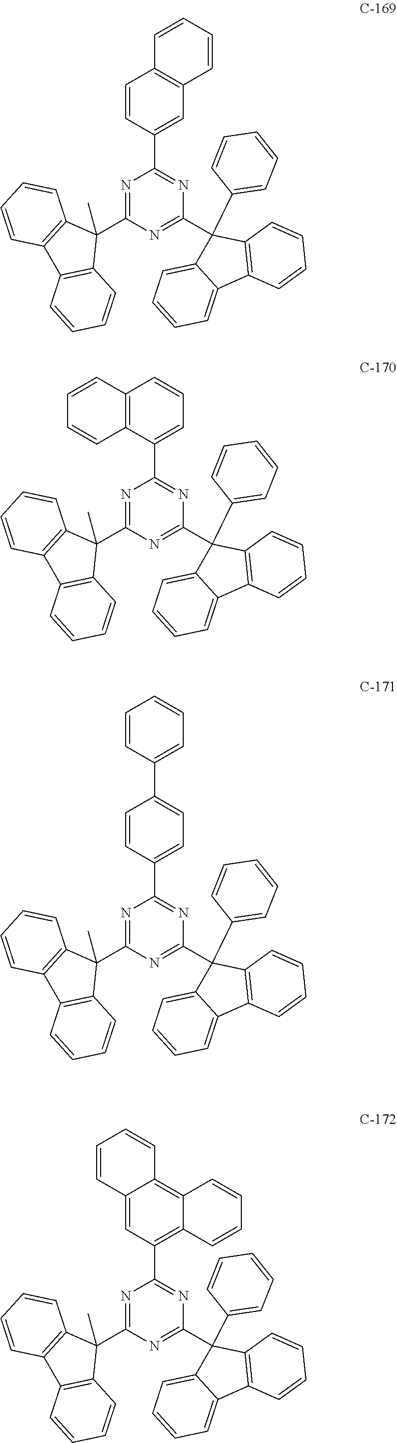 [SMILES: CC1(c2nc(-c3ccc4ccccc4c3)nc(C3(c4ccccc4)c4ccccc4-c4ccccc43)n2)c2ccccc2-c2ccccc21 ; CC1(c2nc(-c3cccc4ccccc34)nc(C3(c4ccccc4)c4ccccc4-c4ccccc43)n2)c2ccccc2-c2ccccc21 ; CC1(c2nc(-c3ccc(-c4ccccc4)cc3)nc(C3(c4ccccc4)c4ccccc4-c4ccccc43)n2)c2ccccc2-c2ccccc21 ; CC1(c2nc(-c3cc4ccccc4c4ccccc34)nc(C3(c4ccccc4)c4ccccc4-c4ccccc43)n2)c2ccccc2-c2ccccc21] 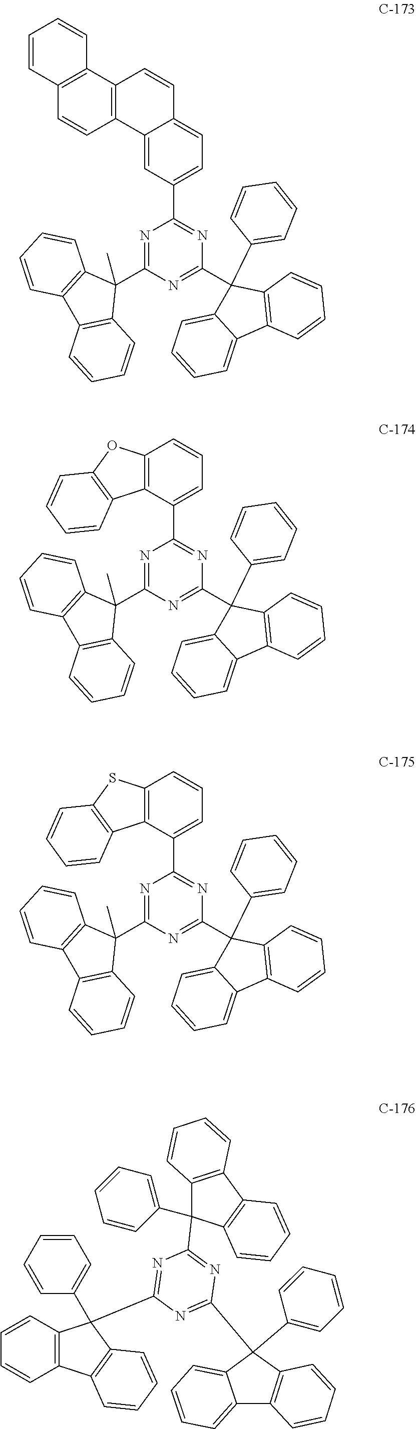 [SMILES: CC1(c2nc(-c3ccc4ccc5c6ccccc6ccc5c4c3)nc(C3(c4ccccc4)c4ccccc4-c4ccccc43)n2)c2ccccc2-c2ccccc21 ; CC1(c2nc(-c3cccc4oc5ccccc5c34)nc(C3(c4ccccc4)c4ccccc4-c4ccccc43)n2)c2ccccc2-c2ccccc21 ; CC1(c2nc(-c3cccc4sc5ccccc5c34)nc(C3(c4ccccc4)c4ccccc4-c4ccccc43)n2)c2ccccc2-c2ccccc21 ; c1ccc(C2(c3nc(C4(c5ccccc5)c5ccccc5-c5ccccc54)nc(C4(c5ccccc5)c5ccccc5-c5ccccc54)n3)c3ccccc3-c3ccccc32)cc1] 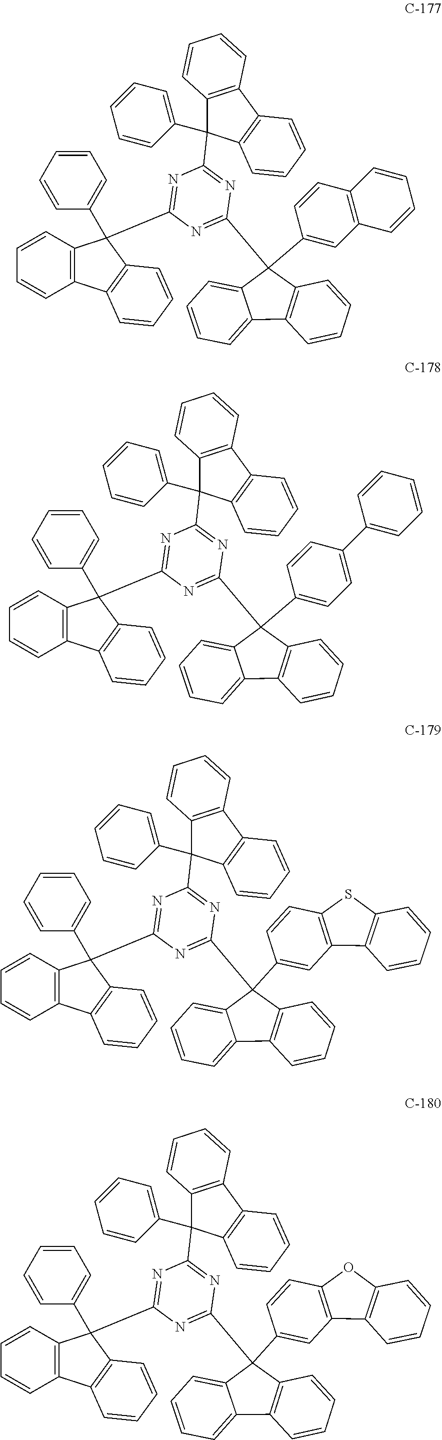 [SMILES: c1ccc(C2(c3nc(C4(c5ccccc5)c5ccccc5-c5ccccc54)nc(C4(c5ccc6ccccc6c5)c5ccccc5-c5ccccc54)n3)c3ccccc3-c3ccccc32)cc1 ; c1ccc(-c2ccc(C3(c4nc(C5(c6ccccc6)c6ccccc6-c6ccccc65)nc(C5(c6ccccc6)c6ccccc6-c6ccccc65)n4)c4ccccc4-c4ccccc43)cc2)cc1 ; c1ccc(C2(c3nc(C4(c5ccccc5)c5ccccc5-c5ccccc54)nc(C4(c5ccc6sc7ccccc7c6c5)c5ccccc5-c5ccccc54)n3)c3ccccc3-c3ccccc32)cc1 ; c1ccc(C2(c3nc(C4(c5ccccc5)c5ccccc5-c5ccccc54)nc(C4(c5ccc6oc7ccccc7c6c5)c5ccccc5-c5ccccc54)n3)c3ccccc3-c3ccccc32)cc1] 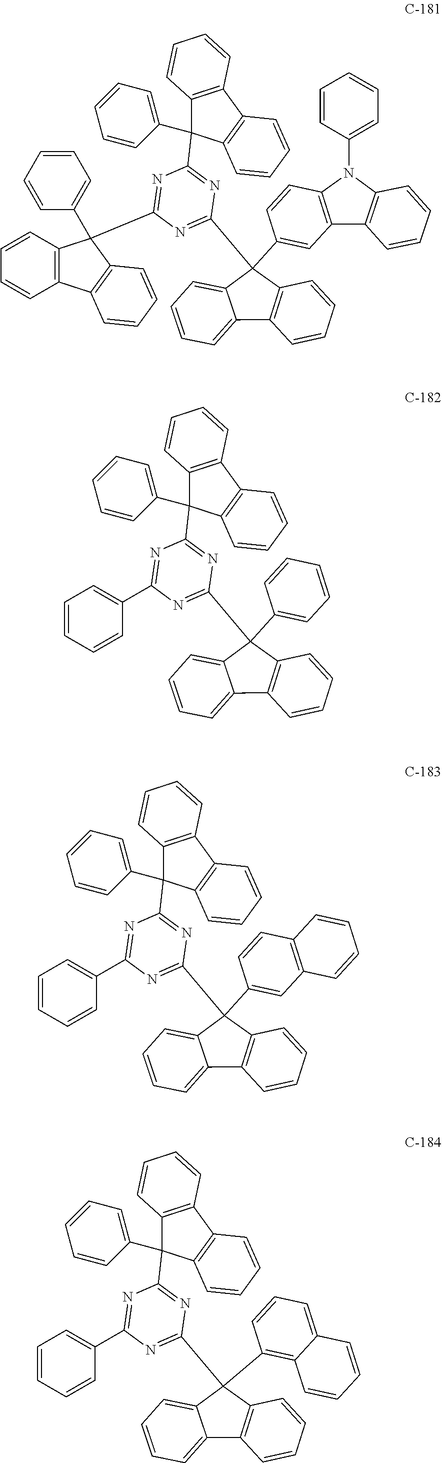 [SMILES: c1ccc(-n2c3ccccc3c3cc(C4(c5nc(C6(c7ccccc7)c7ccccc7-c7ccccc76)nc(C6(c7ccccc7)c7ccccc7-c7ccccc76)n5)c5ccccc5-c5ccccc54)ccc32)cc1 ; c1ccc(-c2nc(C3(c4ccccc4)c4ccccc4-c4ccccc43)nc(C3(c4ccccc4)c4ccccc4-c4ccccc43)n2)cc1 ; c1ccc(-c2nc(C3(c4ccccc4)c4ccccc4-c4ccccc43)nc(C3(c4ccc5ccccc5c4)c4ccccc4-c4ccccc43)n2)cc1 ; c1ccc(-c2nc(C3(c4ccccc4)c4ccccc4-c4ccccc43)nc(C3(c4cccc5ccccc45)c4ccccc4-c4ccccc43)n2)cc1] 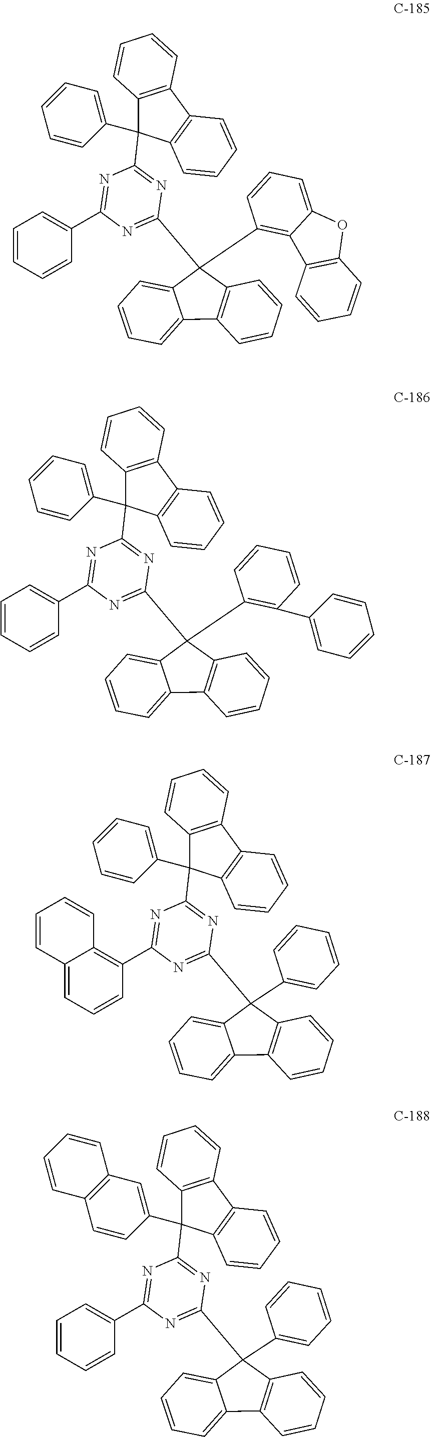 [SMILES: c1ccc(-c2nc(C3(c4ccccc4)c4ccccc4-c4ccccc43)nc(C3(c4cccc5oc6ccccc6c45)c4ccccc4-c4ccccc43)n2)cc1 ; c1ccc(-c2nc(C3(c4ccccc4)c4ccccc4-c4ccccc43)nc(C3(c4ccccc4-c4ccccc4)c4ccccc4-c4ccccc43)n2)cc1 ; c1ccc(C2(c3nc(-c4cccc5ccccc45)nc(C4(c5ccccc5)c5ccccc5-c5ccccc54)n3)c3ccccc3-c3ccccc32)cc1 ; c1ccc(-c2nc(C3(c4ccccc4)c4ccccc4-c4ccccc43)nc(C3(c4ccc5ccccc5c4)c4ccccc4-c4ccccc43)n2)cc1] 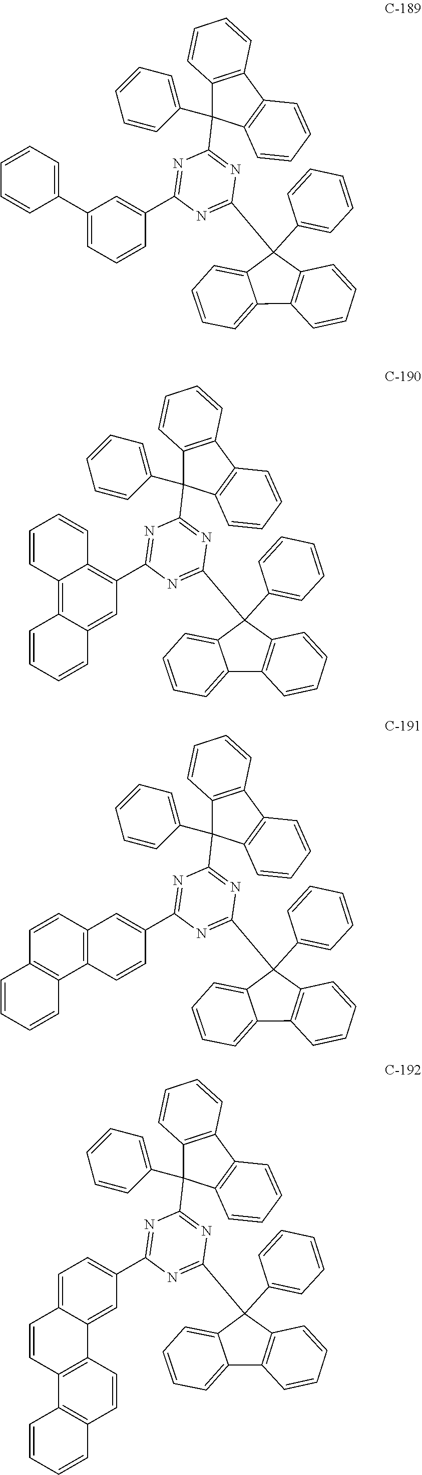 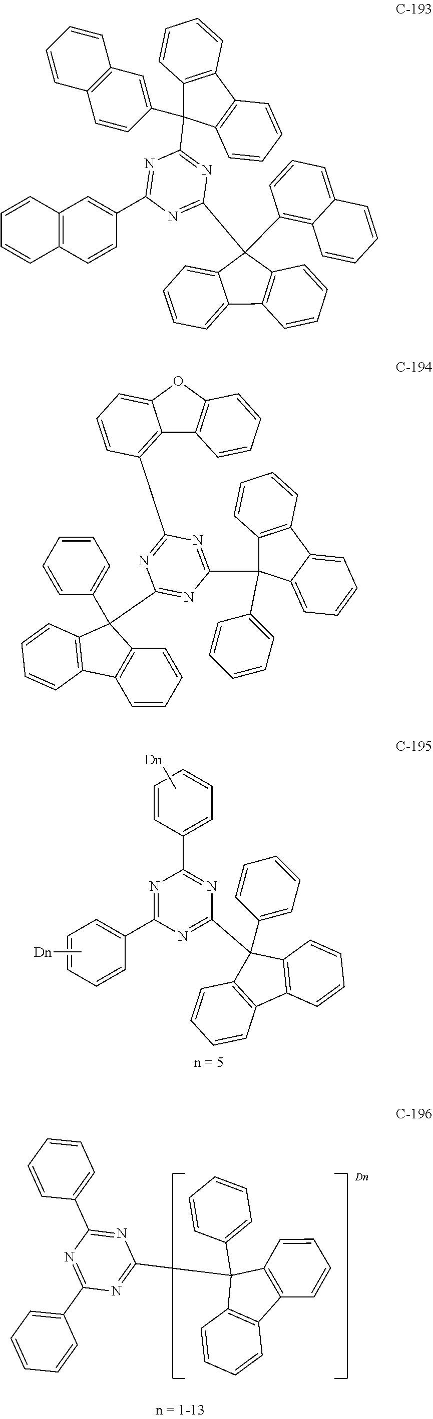 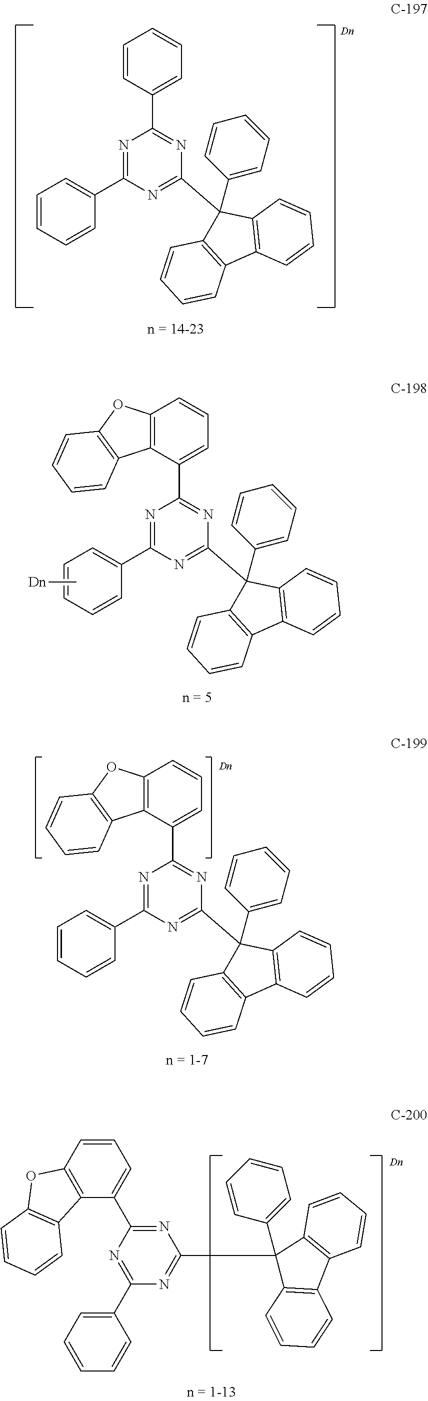 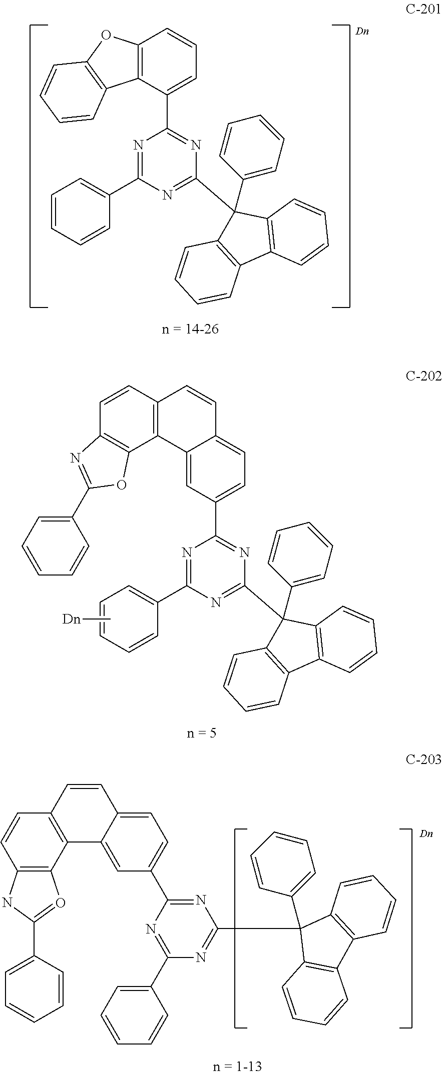 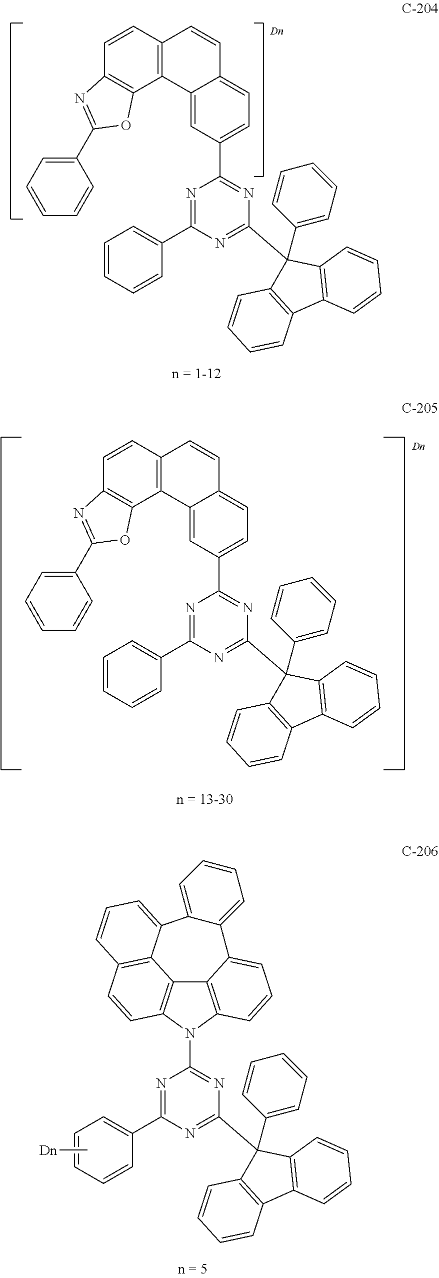 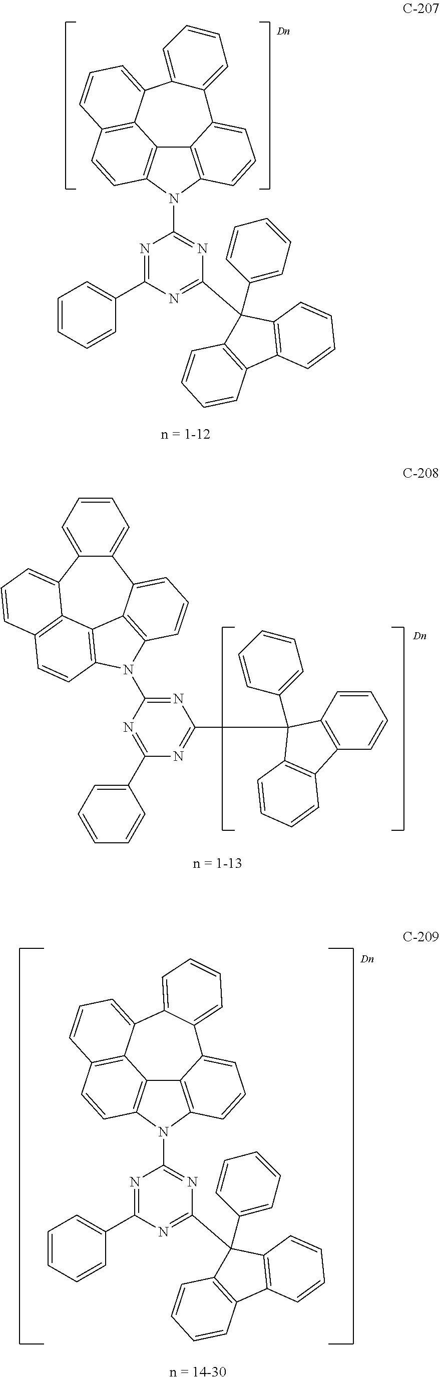 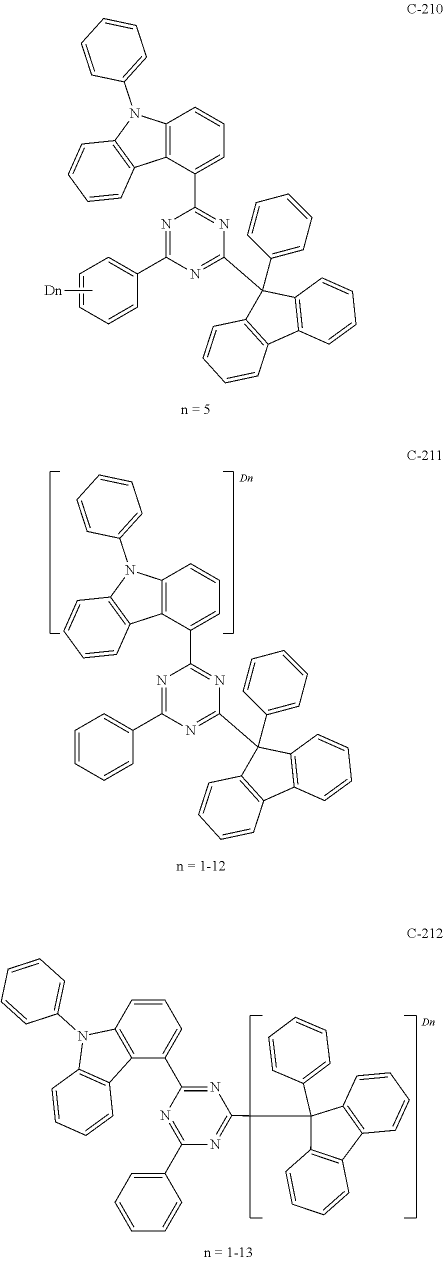 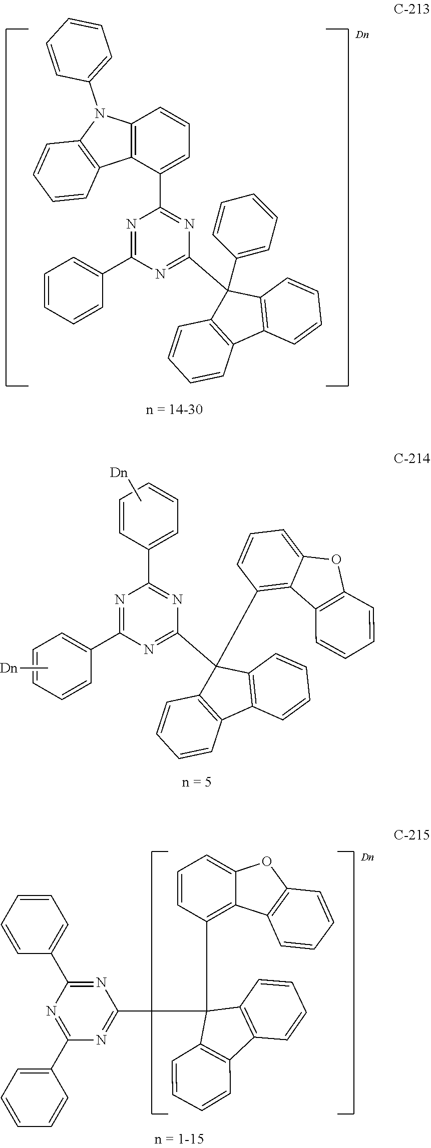 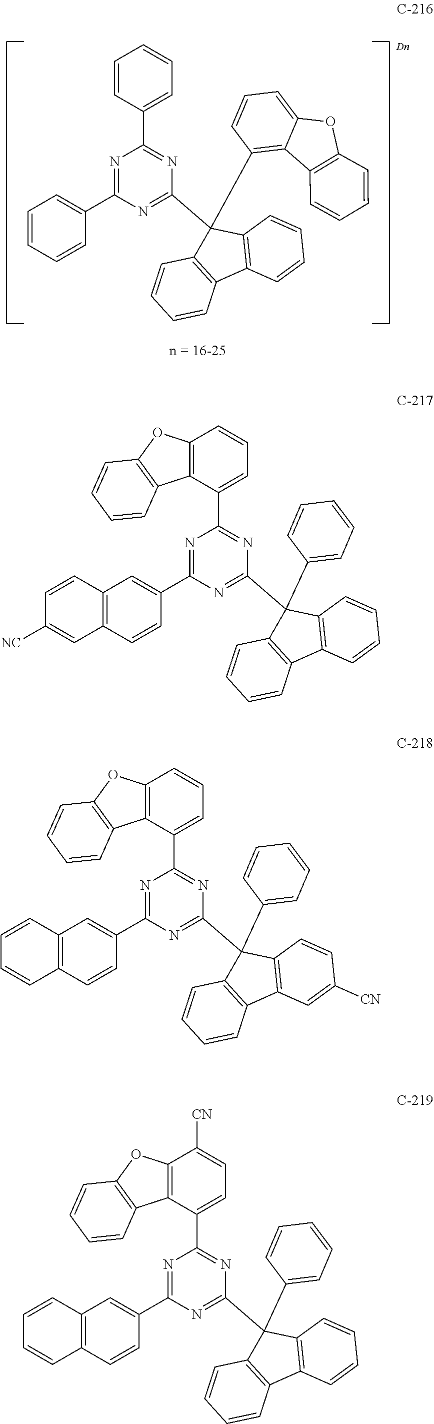 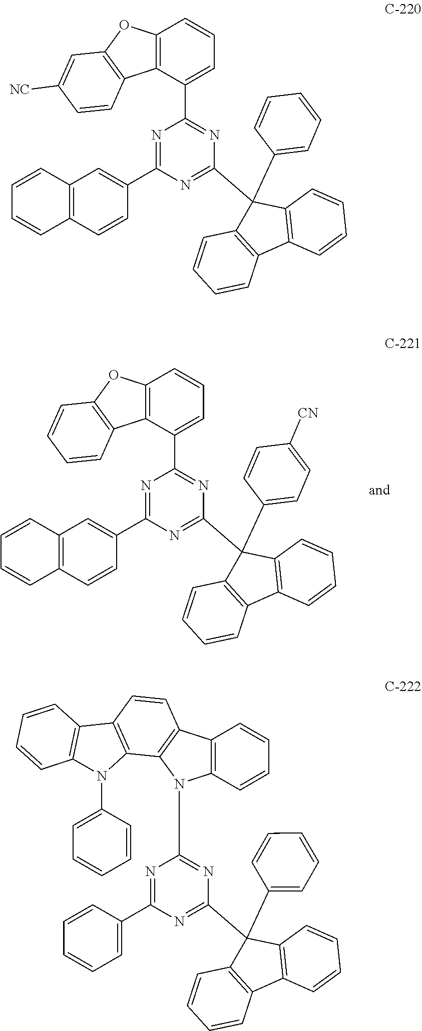 [SMILES: N#Cc1ccc2c(c1)oc1cccc(-c3nc(-c4ccc5ccccc5c4)nc(C4(c5ccccc5)c5ccccc5-c5ccccc54)n3)c12 ; N#Cc1ccc(C2(c3nc(-c4ccc5ccccc5c4)nc(-c4cccc5oc6ccccc6c45)n3)c3ccccc3-c3ccccc32)cc1 ; c1ccc(-c2nc(-n3c4ccccc4c4ccc5c6ccccc6n(-c6ccccc6)c5c43)nc(C3(c4ccccc4)c4ccccc4-c4ccccc43)n2)cc1]
in the compounds above, D represents deuterium, and n represents the number of deuterium.

8. The plurality of host materials according to claim 1, wherein the compound represented by formula 2 is at least one selected from the following compounds:

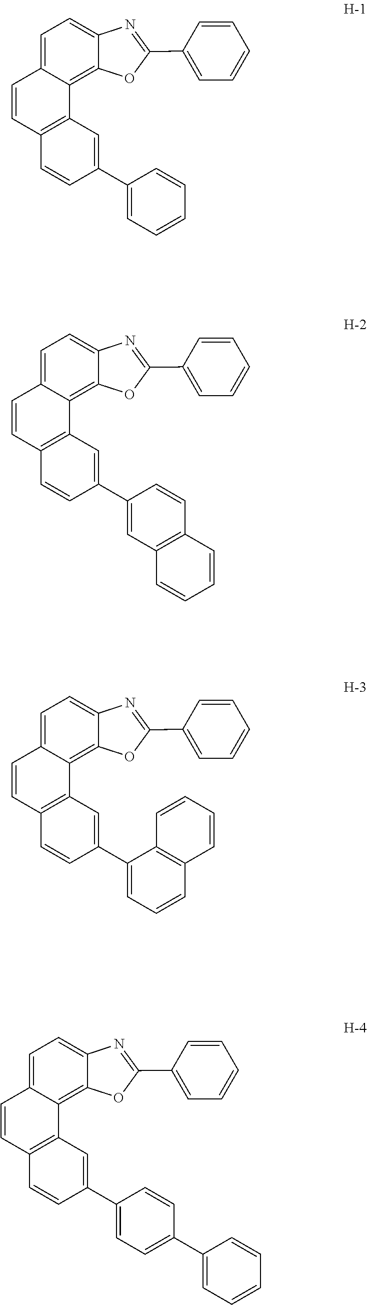 [SMILES: c1ccc(-c2ccc3ccc4ccc5nc(-c6ccccc6)oc5c4c3c2)cc1 ; c1ccc(-c2nc3ccc4ccc5ccc(-c6ccc7ccccc7c6)cc5c4c3o2)cc1 ; c1ccc(-c2nc3ccc4ccc5ccc(-c6cccc7ccccc67)cc5c4c3o2)cc1 ; c1ccc(-c2ccc(-c3ccc4ccc5ccc6nc(-c7ccccc7)oc6c5c4c3)cc2)cc1]

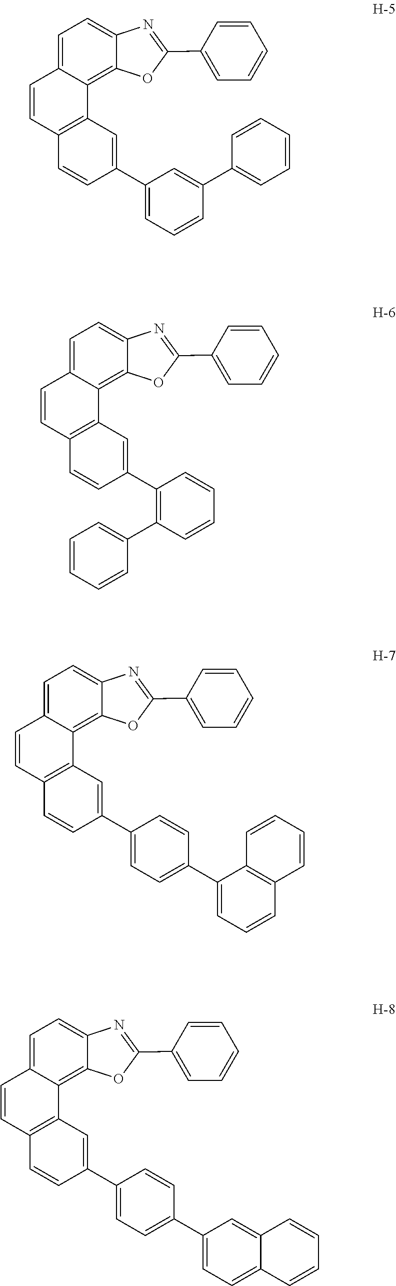 [SMILES: c1ccc(-c2cccc(-c3ccc4ccc5ccc6nc(-c7ccccc7)oc6c5c4c3)c2)cc1 ; c1ccc(-c2nc3ccc4ccc5ccc(-c6ccccc6-c6ccccc6)cc5c4c3o2)cc1 ; c1ccc(-c2nc3ccc4ccc5ccc(-c6ccc(-c7cccc8ccccc78)cc6)cc5c4c3o2)cc1 ; c1ccc(-c2nc3ccc4ccc5ccc(-c6ccc(-c7ccc8ccccc8c7)cc6)cc5c4c3o2)cc1]

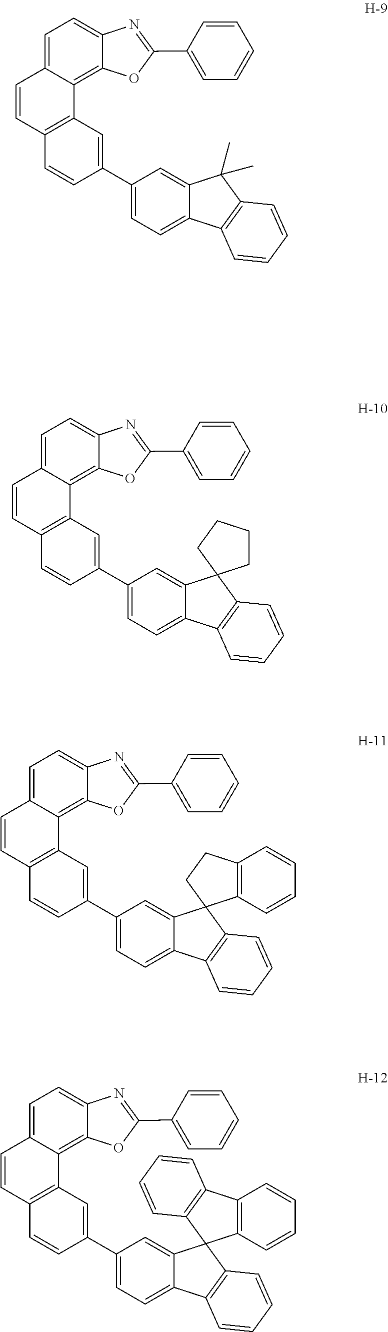 [SMILES: CC1(C)c2ccccc2-c2ccc(-c3ccc4ccc5ccc6nc(-c7ccccc7)oc6c5c4c3)cc21 ; c1ccc(-c2nc3ccc4ccc5ccc(-c6ccc7c(c6)C6(CCCC6)c6ccccc6-7)cc5c4c3o2)cc1 ; c1ccc(-c2nc3ccc4ccc5ccc(-c6ccc7c(c6)C6(CCc8ccccc86)c6ccccc6-7)cc5c4c3o2)cc1 ; c1ccc(-c2nc3ccc4ccc5ccc(-c6ccc7c(c6)C6(c8ccccc8-c8ccccc86)c6ccccc6-7)cc5c4c3o2)cc1]

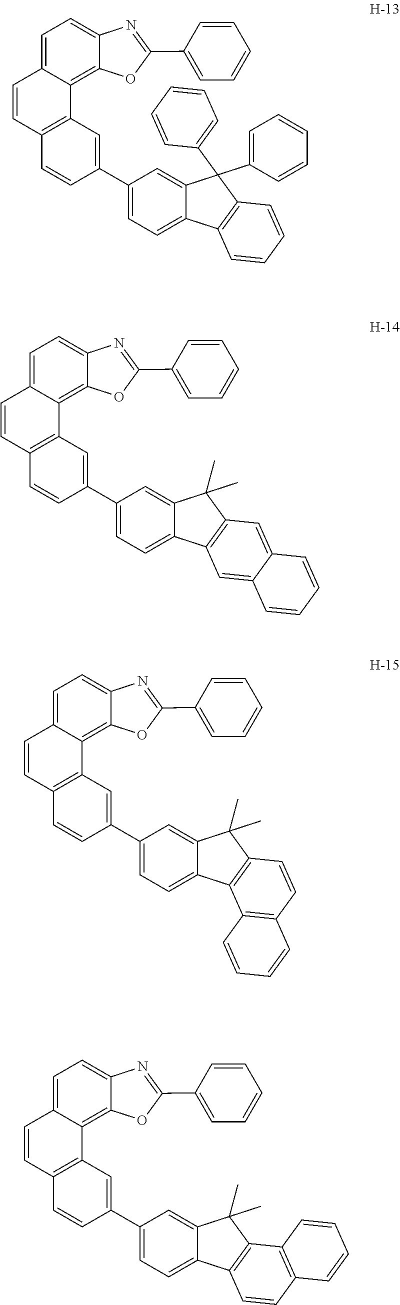 [SMILES: c1ccc(-c2nc3ccc4ccc5ccc(-c6ccc7c(c6)C(c6ccccc6)(c6ccccc6)c6ccccc6-7)cc5c4c3o2)cc1 ; CC1(C)c2cc(-c3ccc4ccc5ccc6nc(-c7ccccc7)oc6c5c4c3)ccc2-c2cc3ccccc3cc21 ; CC1(C)c2cc(-c3ccc4ccc5ccc6nc(-c7ccccc7)oc6c5c4c3)ccc2-c2c1ccc1ccccc21 ; CC1(C)c2cc(-c3ccc4ccc5ccc6nc(-c7ccccc7)oc6c5c4c3)ccc2-c2ccc3ccccc3c21]

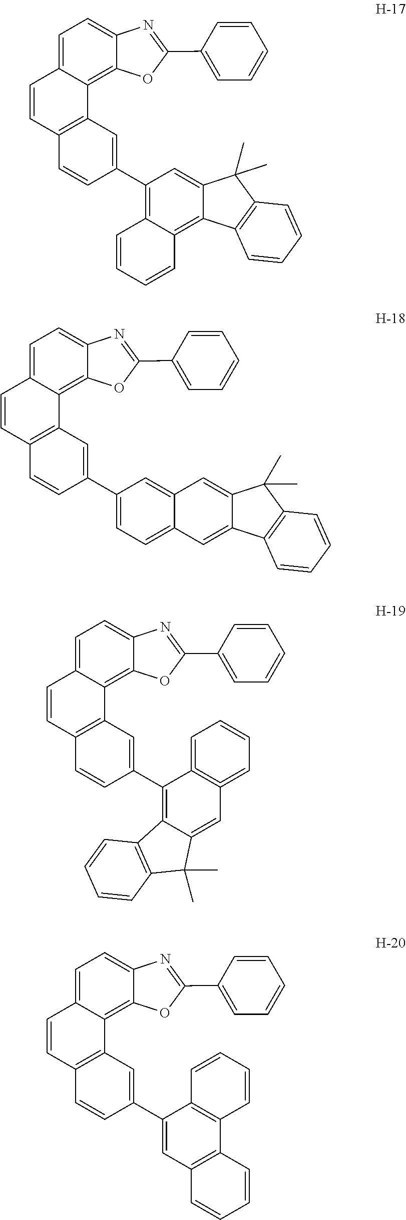

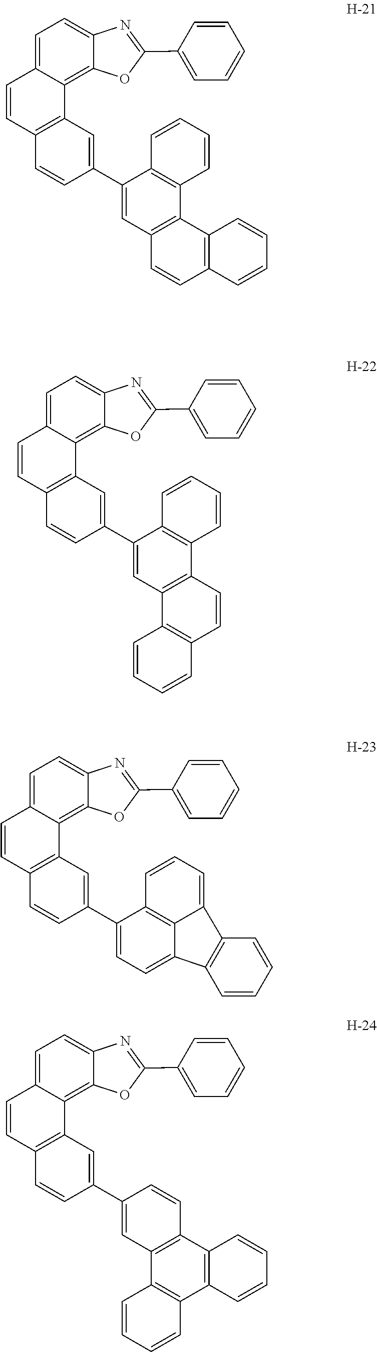

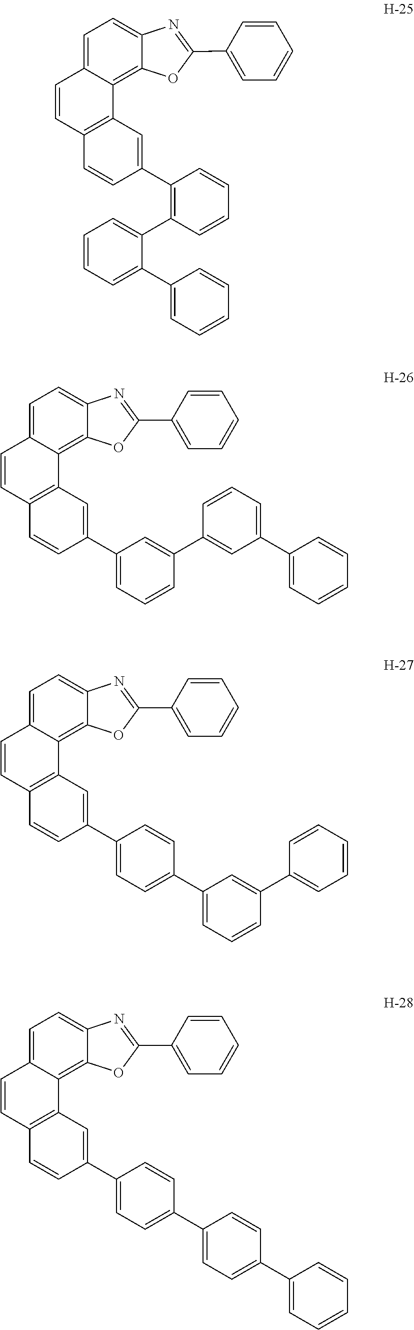 [SMILES: c1ccc(-c2nc3ccc4ccc5ccc(-c6ccccc6-c6ccccc6-c6ccccc6)cc5c4c3o2)cc1 ; c1ccc(-c2cccc(-c3cccc(-c4ccc5ccc6ccc7nc(-c8ccccc8)oc7c6c5c4)c3)c2)cc1 ; c1ccc(-c2cccc(-c3ccc(-c4ccc5ccc6ccc7nc(-c8ccccc8)oc7c6c5c4)cc3)c2)cc1 ; c1ccc(-c2ccc(-c3ccc(-c4ccc5ccc6ccc7nc(-c8ccccc8)oc7c6c5c4)cc3)cc2)cc1]

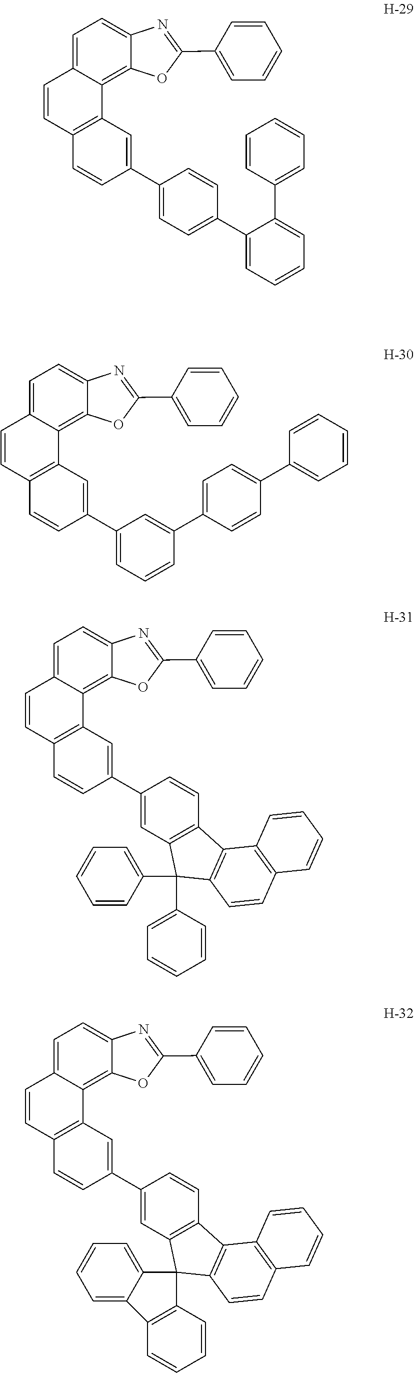 [SMILES: c1ccc(-c2nc3ccc4ccc5ccc(-c6ccc(-c7ccccc7-c7ccccc7)cc6)cc5c4c3o2)cc1 ; c1ccc(-c2ccc(-c3cccc(-c4ccc5ccc6ccc7nc(-c8ccccc8)oc7c6c5c4)c3)cc2)cc1 ; c1ccc(-c2nc3ccc4ccc5ccc(-c6ccc7c(c6)C(c6ccccc6)(c6ccccc6)c6ccc8ccccc8c6-7)cc5c4c3o2)cc1 ; c1ccc(-c2nc3ccc4ccc5ccc(-c6ccc7c(c6)C6(c8ccccc8-c8ccccc86)c6ccc8ccccc8c6-7)cc5c4c3o2)cc1]

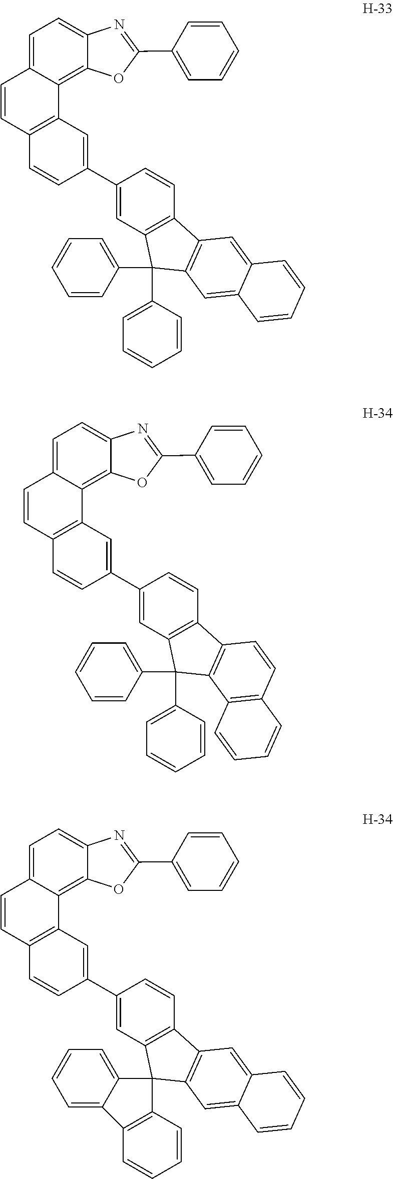 [SMILES: c1ccc(-c2nc3ccc4ccc5ccc(-c6ccc7c(c6)C(c6ccccc6)(c6ccccc6)c6cc8ccccc8cc6-7)cc5c4c3o2)cc1 ; c1ccc(-c2nc3ccc4ccc5ccc(-c6ccc7c(c6)C(c6ccccc6)(c6ccccc6)c6c-7ccc7ccccc67)cc5c4c3o2)cc1 ; c1ccc(-c2nc3ccc4ccc5ccc(-c6ccc7c(c6)C6(c8ccccc8-c8ccccc86)c6cc8ccccc8cc6-7)cc5c4c3o2)cc1]

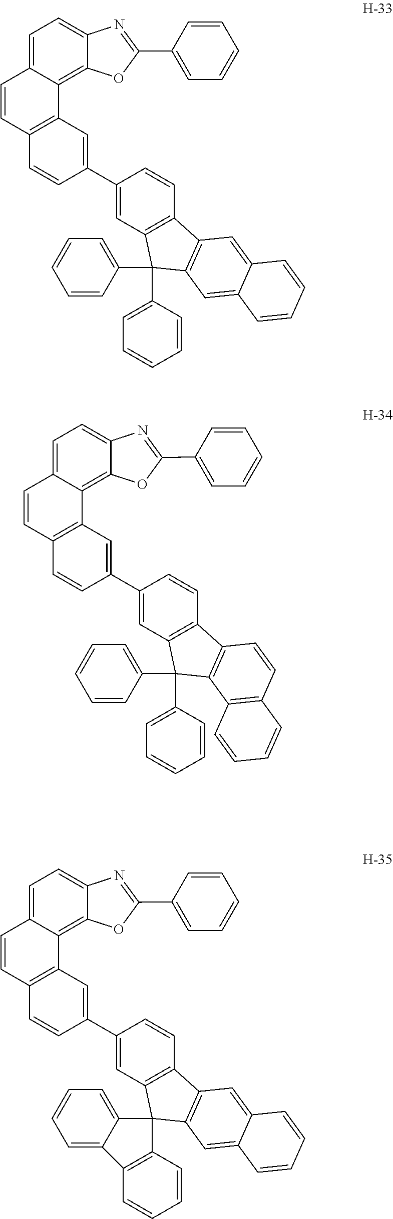 [SMILES: c1ccc(-c2nc3ccc4ccc5ccc(-c6ccc7c(c6)C(c6ccccc6)(c6ccccc6)c6cc8ccccc8cc6-7)cc5c4c3o2)cc1 ; c1ccc(-c2nc3ccc4ccc5ccc(-c6ccc7c(c6)C(c6ccccc6)(c6ccccc6)c6c-7ccc7ccccc67)cc5c4c3o2)cc1 ; c1ccc(-c2nc3ccc4ccc5ccc(-c6ccc7c(c6)C6(c8ccccc8-c8ccccc86)c6cc8ccccc8cc6-7)cc5c4c3o2)cc1]

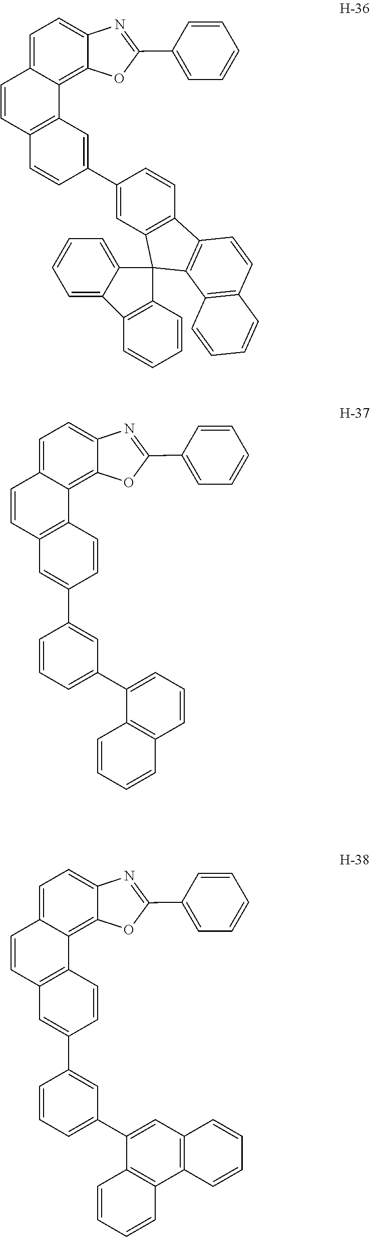 [SMILES: c1ccc(-c2nc3ccc4ccc5ccc(-c6ccc7c(c6)C6(c8ccccc8-c8ccccc86)c6c-7ccc7ccccc67)cc5c4c3o2)cc1 ; c1ccc(-c2nc3ccc4ccc5cc(-c6cccc(-c7cccc8ccccc78)c6)ccc5c4c3o2)cc1 ; c1ccc(-c2nc3ccc4ccc5cc(-c6cccc(-c7cc8ccccc8c8ccccc78)c6)ccc5c4c3o2)cc1]

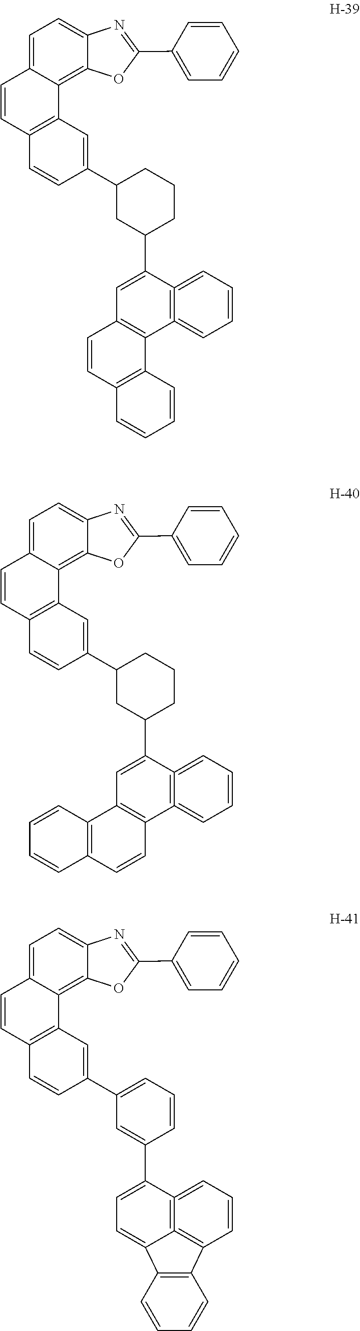

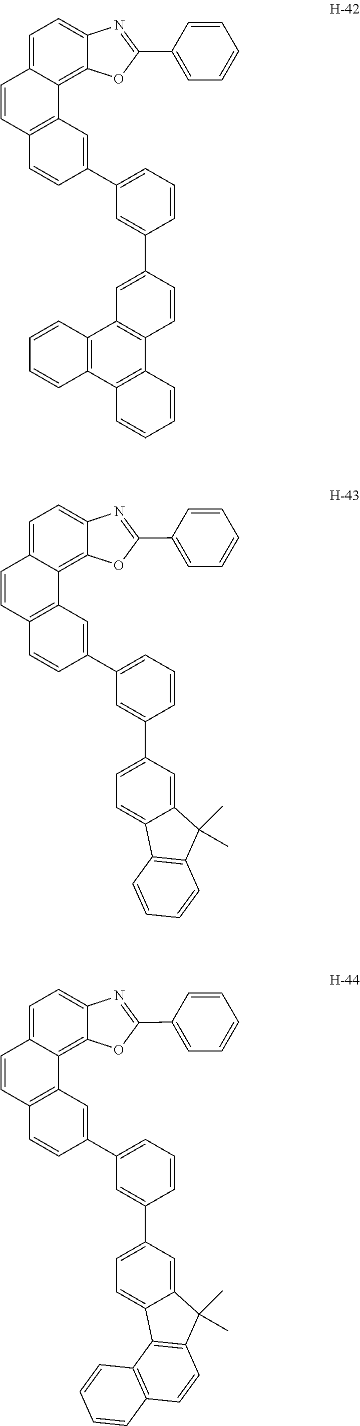 [SMILES: c1ccc(-c2nc3ccc4ccc5ccc(-c6cccc(-c7ccc8c9ccccc9c9ccccc9c8c7)c6)cc5c4c3o2)cc1 ; CC1(C)c2ccccc2-c2ccc(-c3cccc(-c4ccc5ccc6ccc7nc(-c8ccccc8)oc7c6c5c4)c3)cc21 ; CC1(C)c2cc(-c3cccc(-c4ccc5ccc6ccc7nc(-c8ccccc8)oc7c6c5c4)c3)ccc2-c2c1ccc1ccccc21]

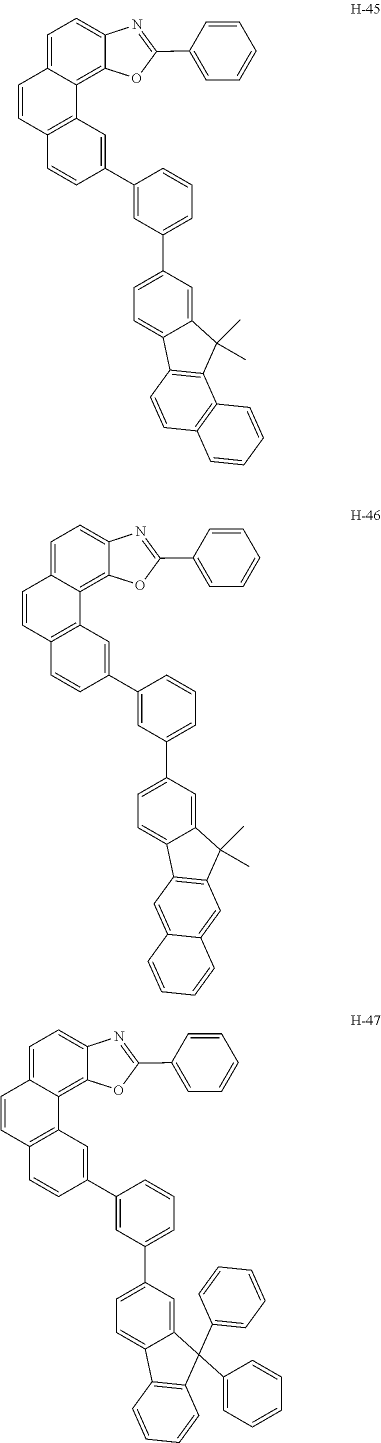

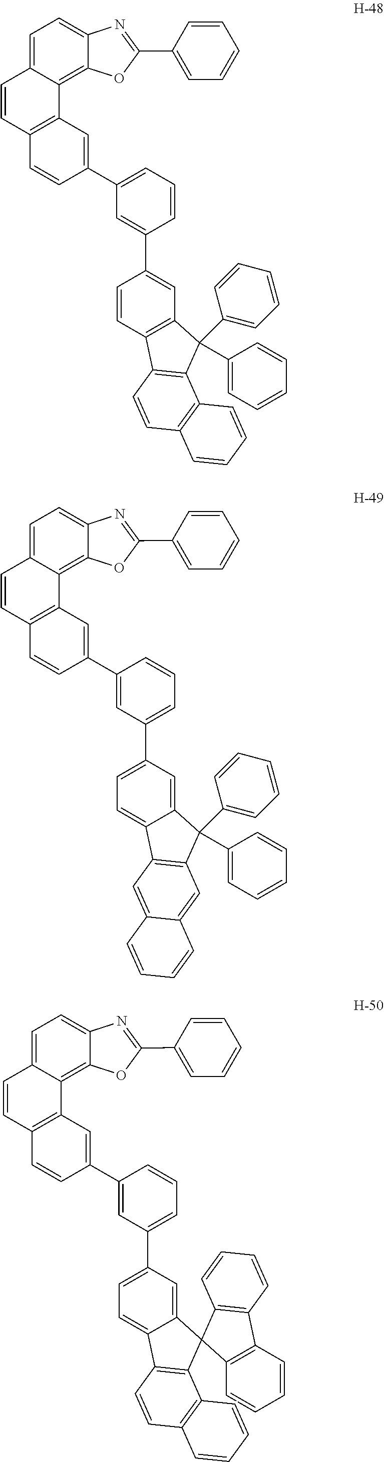

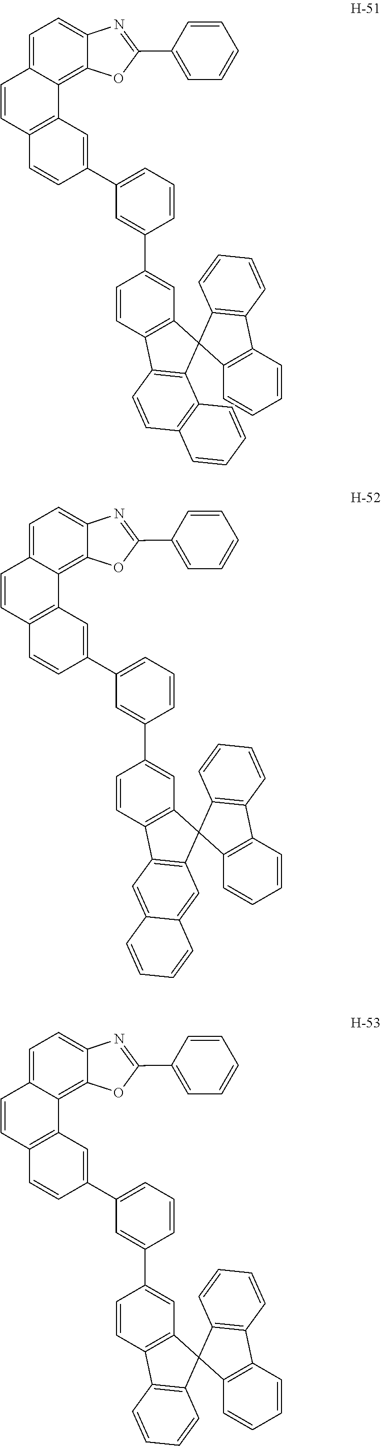

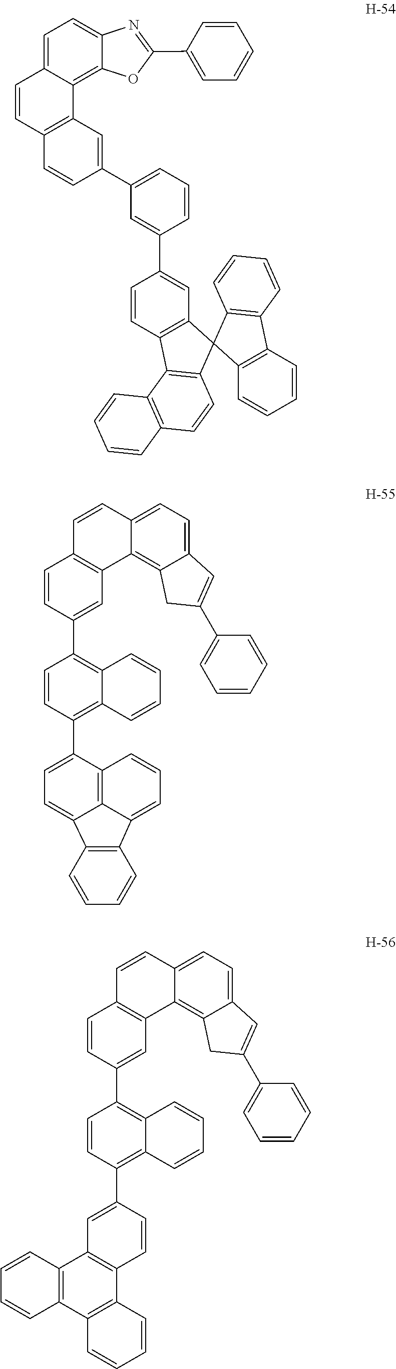

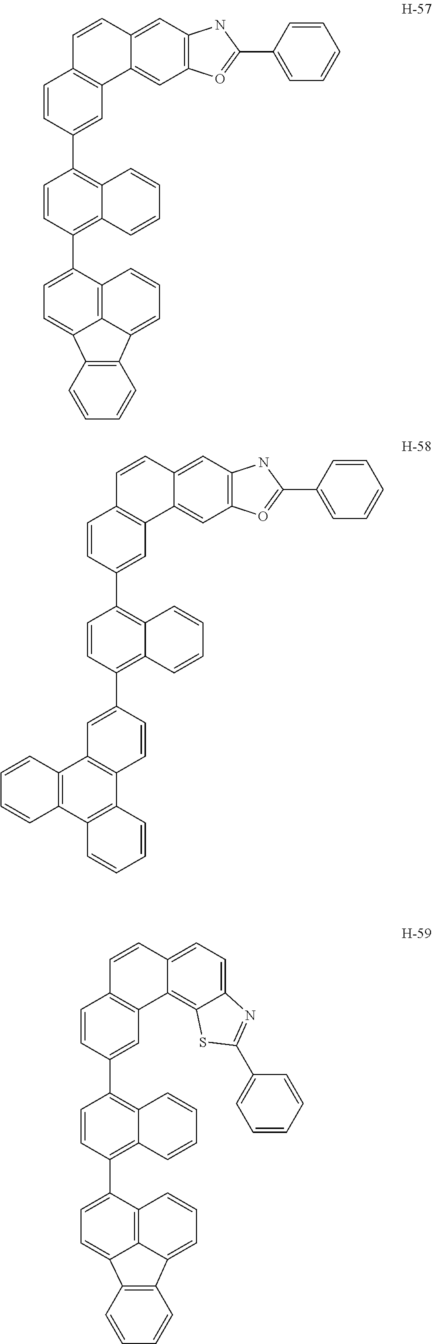

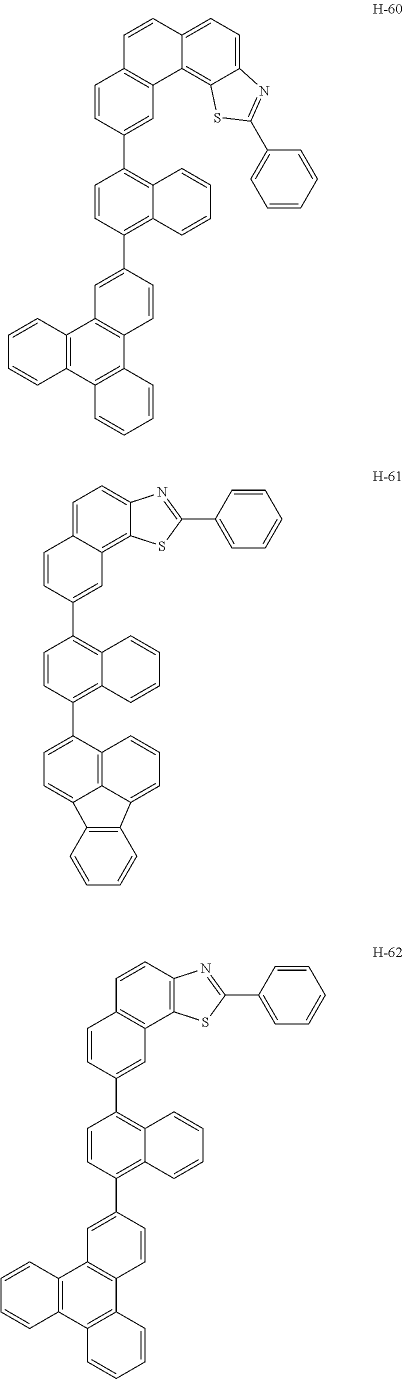 [SMILES: c1ccc(-c2nc3ccc4ccc5ccc(-c6ccc(-c7ccc8c9ccccc9c9ccccc9c8c7)c7ccccc67)cc5c4c3s2)cc1 ; c1ccc(-c2nc3ccc4ccc(-c5ccc(-c6ccc7c8c(cccc68)-c6ccccc6-7)c6ccccc56)cc4c3s2)cc1 ; c1ccc(-c2nc3ccc4ccc(-c5ccc(-c6ccc7c8ccccc8c8ccccc8c7c6)c6ccccc56)cc4c3s2)cc1]

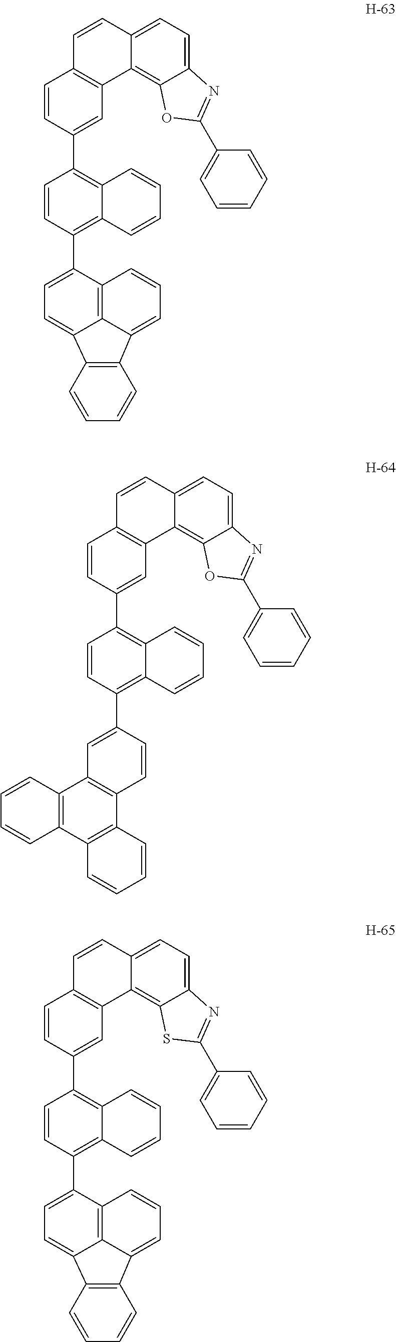 [SMILES: c1ccc(-c2nc3ccc4ccc5ccc(-c6ccc(-c7ccc8c9c(cccc79)-c7ccccc7-8)c7ccccc67)cc5c4c3o2)cc1 ; c1ccc(-c2nc3ccc4ccc5ccc(-c6ccc(-c7ccc8c9ccccc9c9ccccc9c8c7)c7ccccc67)cc5c4c3o2)cc1 ; c1ccc(-c2nc3ccc4ccc5ccc(-c6ccc(-c7ccc8c9c(cccc79)-c7ccccc7-8)c7ccccc67)cc5c4c3s2)cc1]

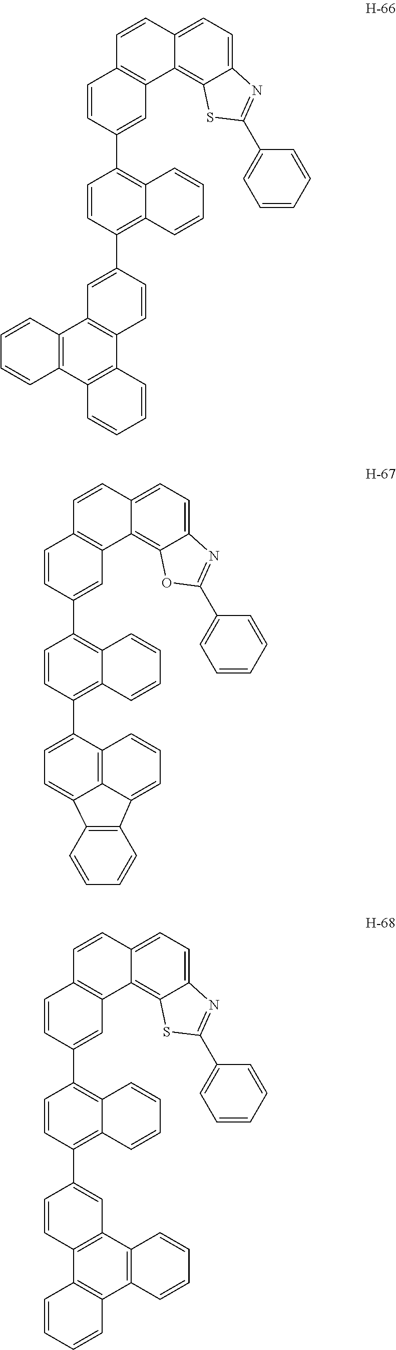 [SMILES: c1ccc(-c2nc3ccc4ccc5ccc(-c6ccc(-c7ccc8c9ccccc9c9ccccc9c8c7)c7ccccc67)cc5c4c3s2)cc1 ; c1ccc(-c2nc3ccc4ccc5ccc(-c6ccc(-c7ccc8c9c(cccc79)-c7ccccc7-8)c7ccccc67)cc5c4c3o2)cc1 ; c1ccc(-c2nc3ccc4ccc5ccc(-c6ccc(-c7ccc8c9ccccc9c9ccccc9c8c7)c7ccccc67)cc5c4c3s2)cc1]

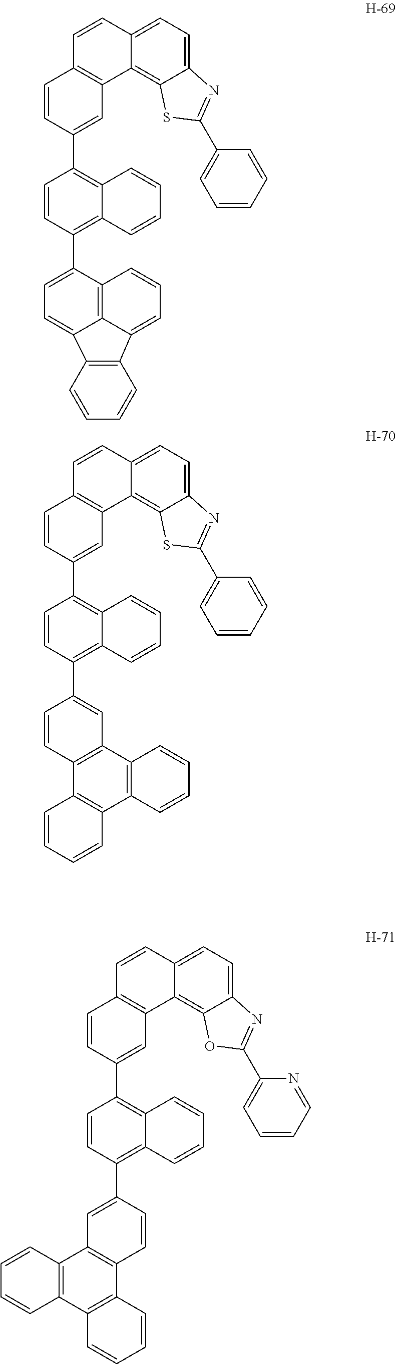

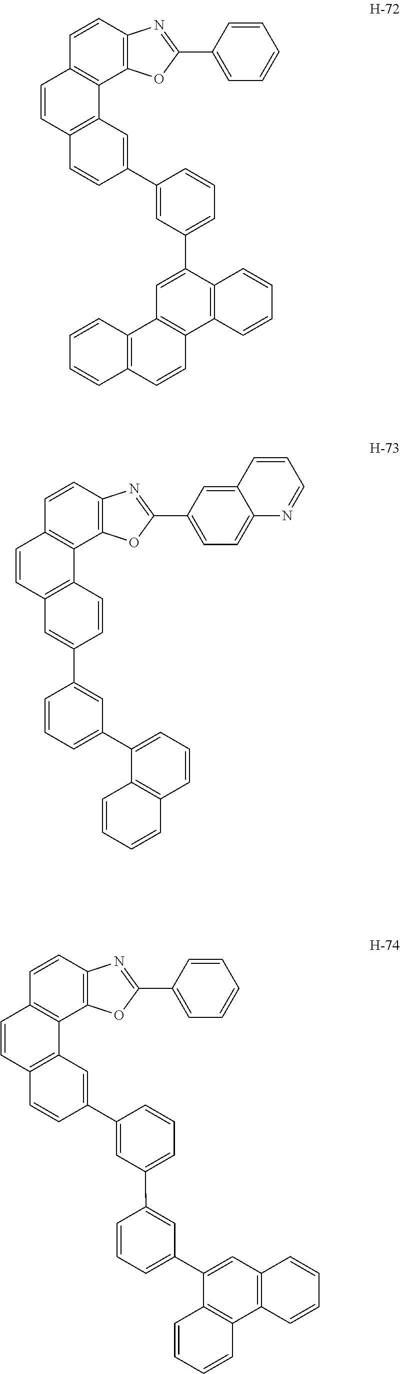 [SMILES: c1ccc(-c2nc3ccc4ccc5ccc(-c6cccc(-c7cc8c9ccccc9ccc8c8ccccc78)c6)cc5c4c3o2)cc1 ; c1cc(-c2ccc3c(ccc4ccc5nc(-c6ccc7ncccc7c6)oc5c43)c2)cc(-c2cccc3ccccc23)c1 ; c1ccc(-c2nc3ccc4ccc5ccc(-c6cccc(-c7cccc(-c8cc9ccccc9c9ccccc89)c7)c6)cc5c4c3o2)cc1]

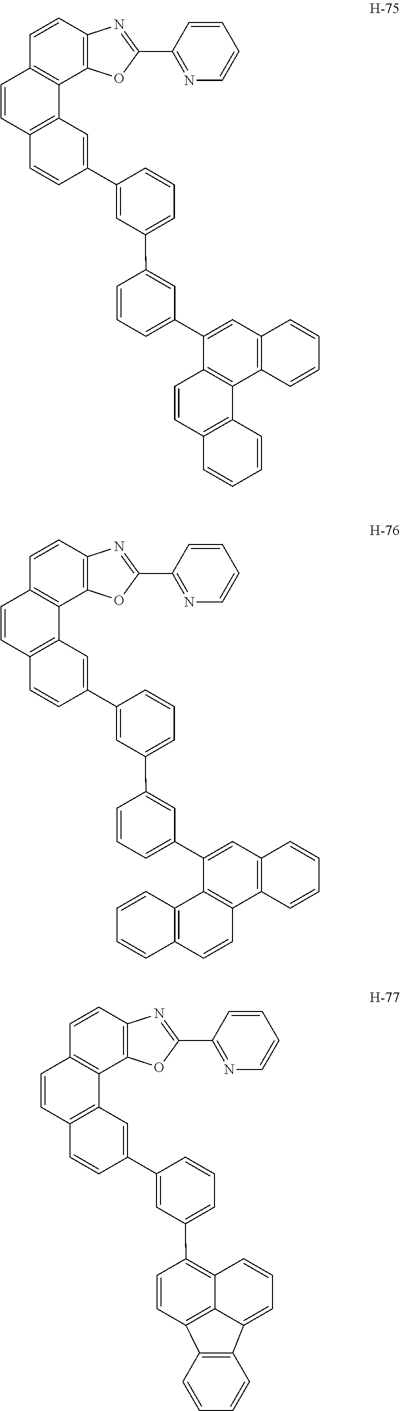 [SMILES: c1ccc(-c2nc3ccc4ccc5ccc(-c6cccc(-c7cccc(-c8cc9ccccc9c9c8ccc8ccccc89)c7)c6)cc5c4c3o2)nc1 ; c1ccc(-c2nc3ccc4ccc5ccc(-c6cccc(-c7cccc(-c8cc9ccccc9c9ccc%10ccccc%10c89)c7)c6)cc5c4c3o2)nc1 ; c1ccc(-c2nc3ccc4ccc5ccc(-c6cccc(-c7ccc8c9c(cccc79)-c7ccccc7-8)c6)cc5c4c3o2)nc1]

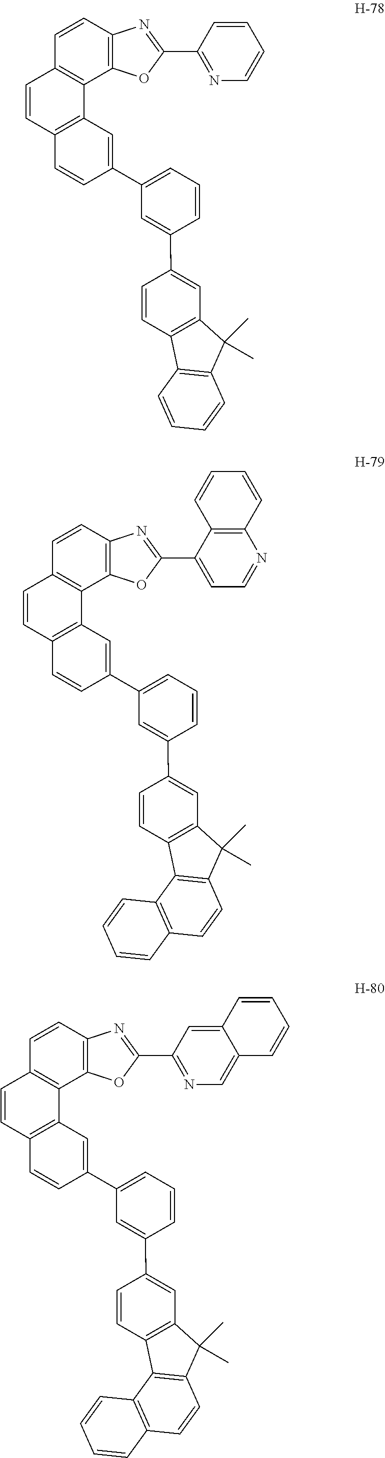

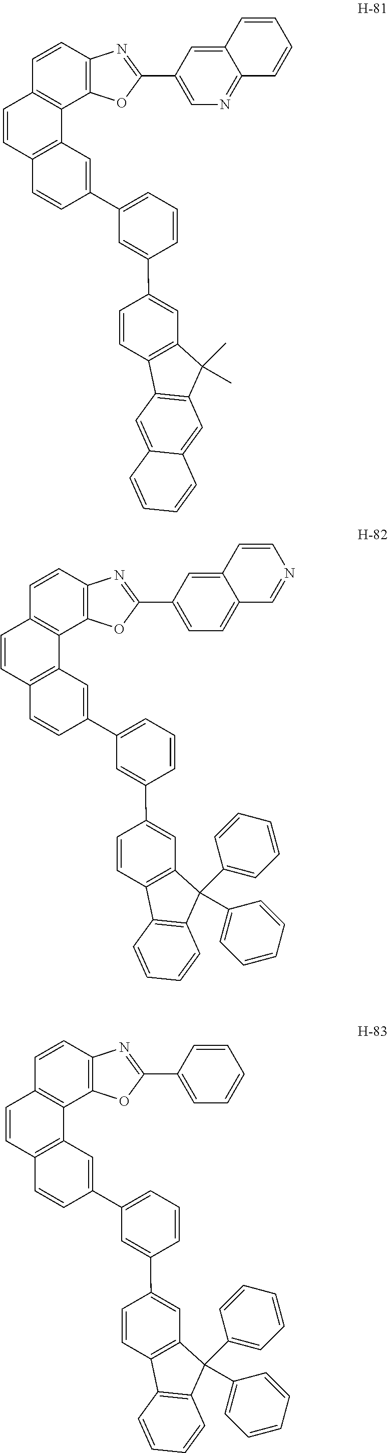 [SMILES: CC1(C)c2cc(-c3cccc(-c4ccc5ccc6ccc7nc(-c8cnc9ccccc9c8)oc7c6c5c4)c3)ccc2-c2cc3ccccc3cc21 ; c1ccc(C2(c3ccccc3)c3ccccc3-c3ccc(-c4cccc(-c5ccc6ccc7ccc8nc(-c9ccc%10cnccc%10c9)oc8c7c6c5)c4)cc32)cc1 ; c1ccc(-c2nc3ccc4ccc5ccc(-c6cccc(-c7ccc8c(c7)C(c7ccccc7)(c7ccccc7)c7ccccc7-8)c6)cc5c4c3o2)cc1]

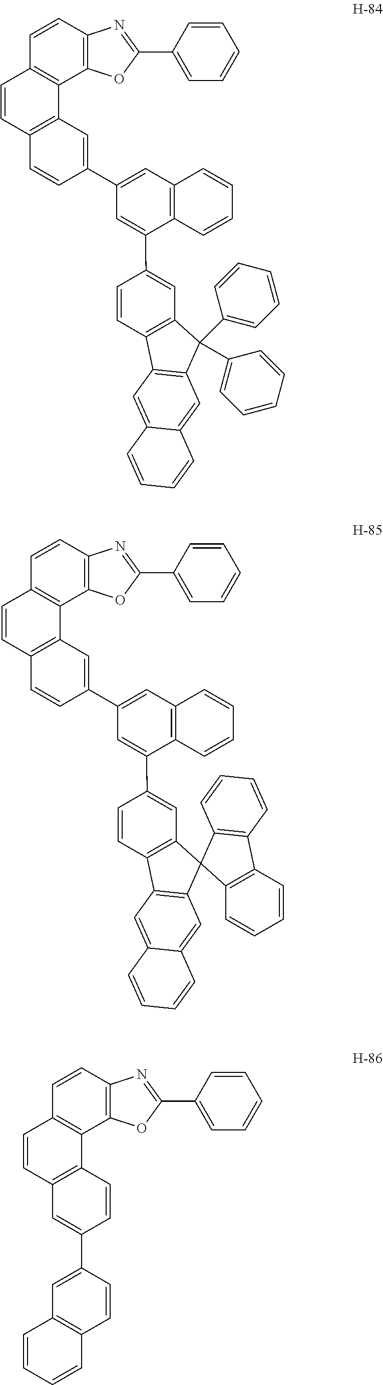 [SMILES: c1ccc(-c2nc3ccc4ccc5ccc(-c6cc(-c7ccc8c(c7)C(c7ccccc7)(c7ccccc7)c7cc9ccccc9cc7-8)c7ccccc7c6)cc5c4c3o2)cc1 ; c1ccc(-c2nc3ccc4ccc5ccc(-c6cc(-c7ccc8c(c7)C7(c9ccccc9-c9ccccc97)c7cc9ccccc9cc7-8)c7ccccc7c6)cc5c4c3o2)cc1 ; c1ccc(-c2nc3ccc4ccc5cc(-c6ccc7ccccc7c6)ccc5c4c3o2)cc1]

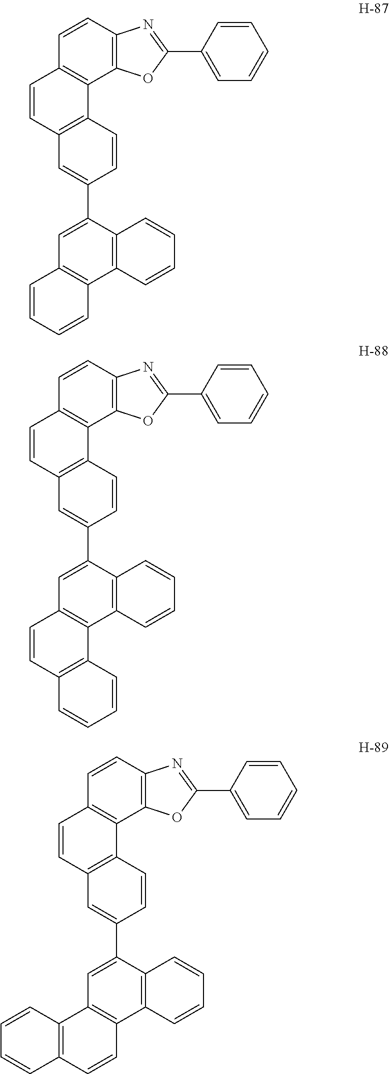

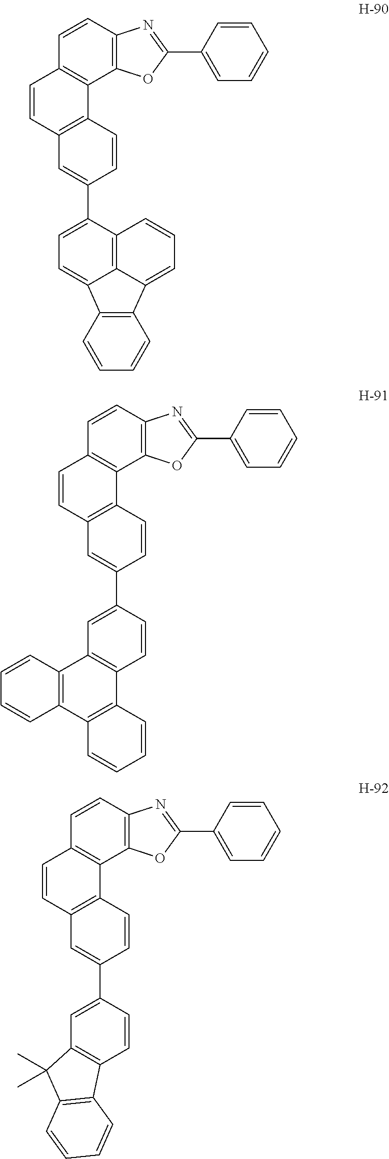

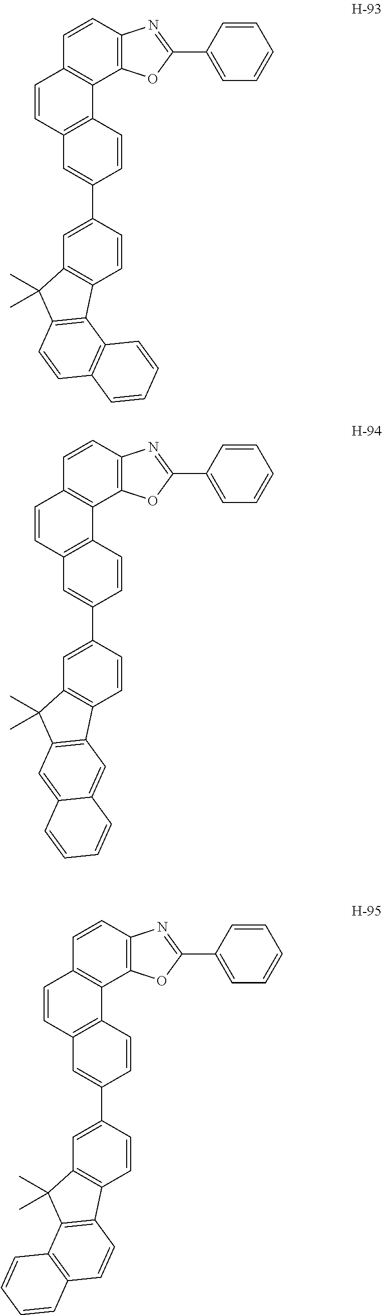

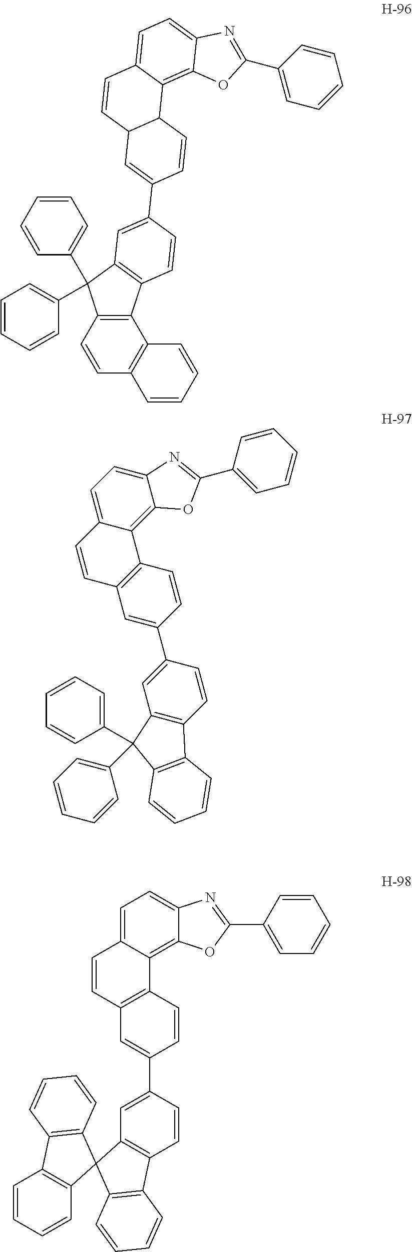 [SMILES: C1=CC2c3c(ccc4nc(-c5ccccc5)oc34)C=CC2C=C1c1ccc2c(c1)C(c1ccccc1)(c1ccccc1)c1ccc3ccccc3c1-2 ; c1ccc(-c2nc3ccc4ccc5cc(-c6ccc7c(c6)C(c6ccccc6)(c6ccccc6)c6ccccc6-7)ccc5c4c3o2)cc1 ; c1ccc(-c2nc3ccc4ccc5cc(-c6ccc7c(c6)C6(c8ccccc8-c8ccccc86)c6ccccc6-7)ccc5c4c3o2)cc1]

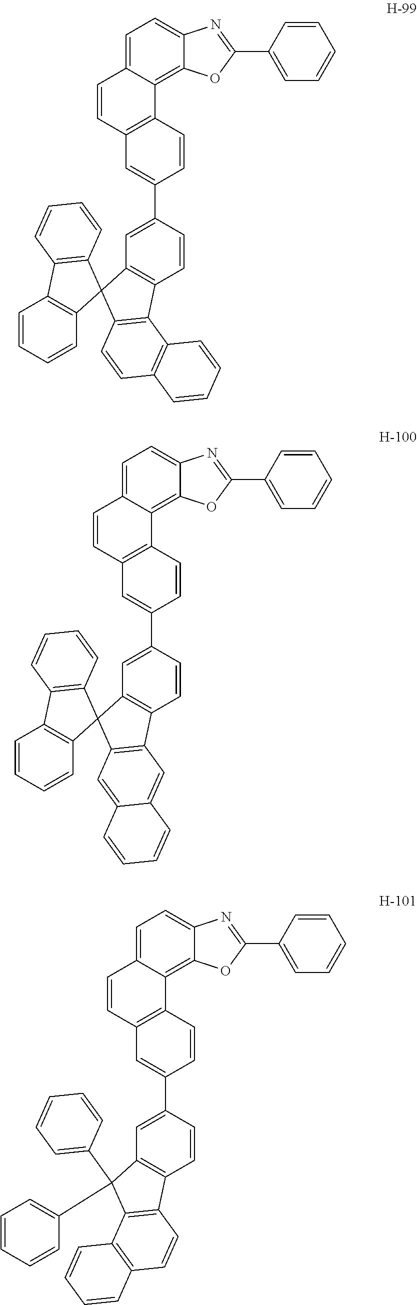 [SMILES: c1ccc(-c2nc3ccc4ccc5cc(-c6ccc7c(c6)C6(c8ccccc8-c8ccccc86)c6ccc8ccccc8c6-7)ccc5c4c3o2)cc1 ; c1ccc(-c2nc3ccc4ccc5cc(-c6ccc7c(c6)C6(c8ccccc8-c8ccccc86)c6cc8ccccc8cc6-7)ccc5c4c3o2)cc1 ; c1ccc(-c2nc3ccc4ccc5cc(-c6ccc7c(c6)C(c6ccccc6)(c6ccccc6)c6c-7ccc7ccccc67)ccc5c4c3o2)cc1]

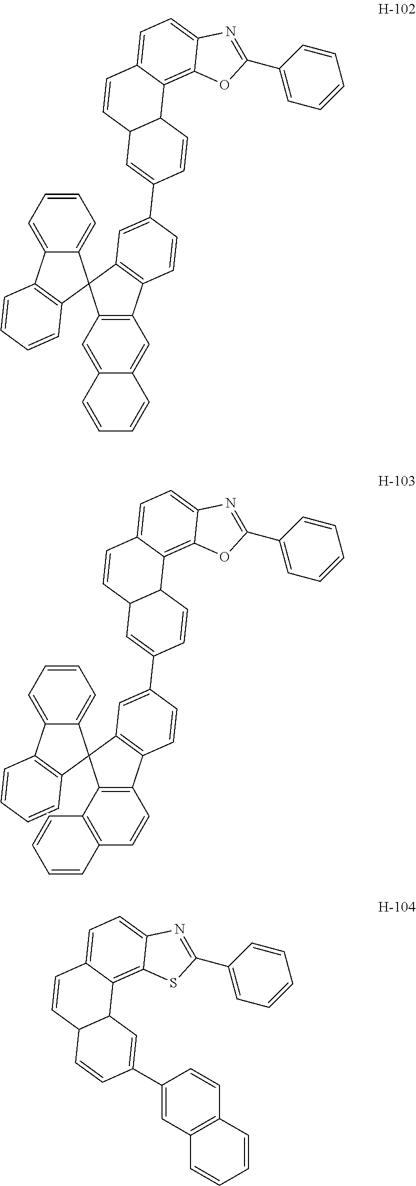 [SMILES: C1=CC2c3c(ccc4nc(-c5ccccc5)oc34)C=CC2C=C1c1ccc2c(c1)C1(c3ccccc3-c3ccccc31)c1cc3ccccc3cc1-2 ; C1=CC2c3c(ccc4nc(-c5ccccc5)oc34)C=CC2C=C1c1ccc2c(c1)C1(c3ccccc3-c3ccccc31)c1c-2ccc2ccccc12 ; C1=CC2C=Cc3ccc4nc(-c5ccccc5)sc4c3C2C=C1c1ccc2ccccc2c1]

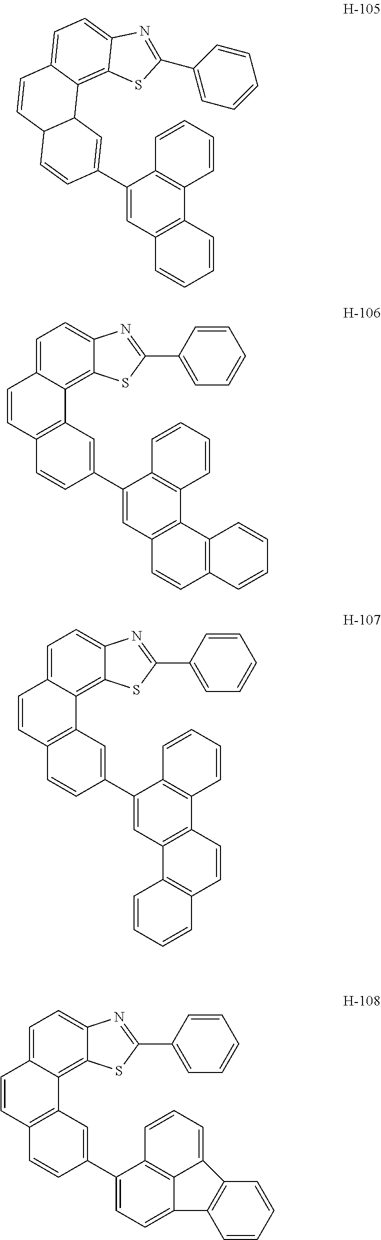

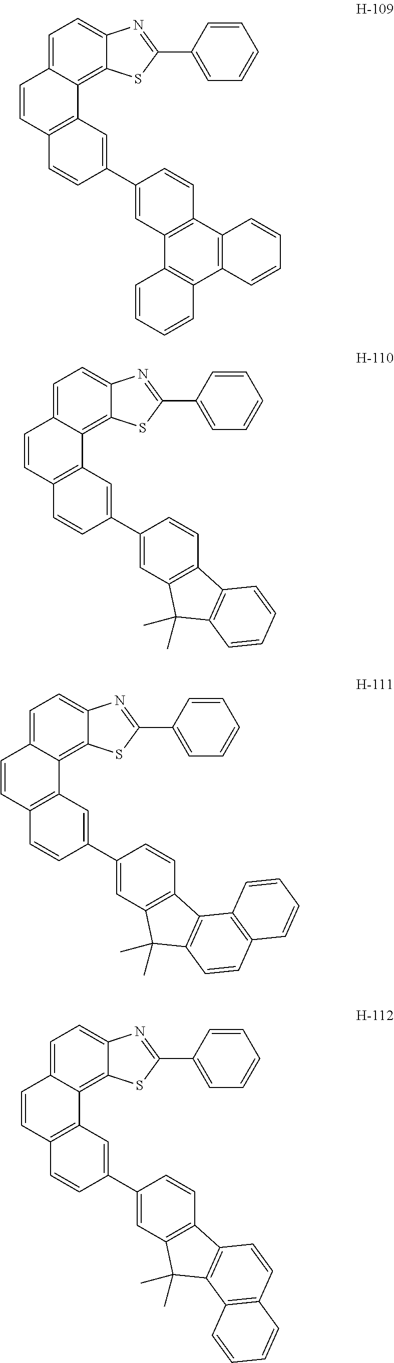

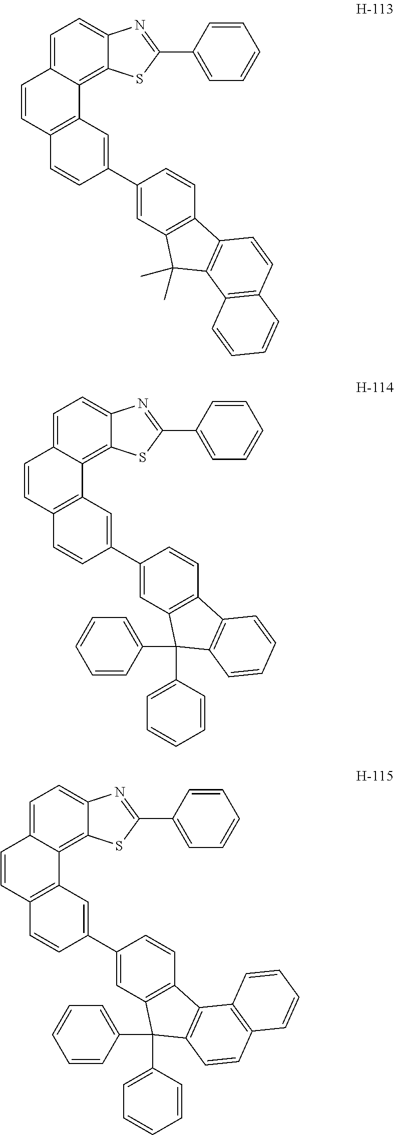

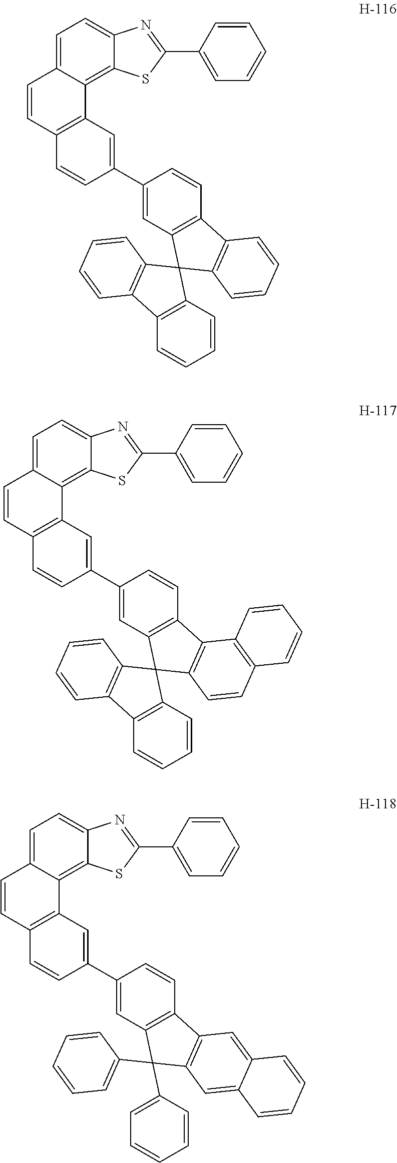 [SMILES: c1ccc(-c2nc3ccc4ccc5ccc(-c6ccc7c(c6)C6(c8ccccc8-c8ccccc86)c6ccccc6-7)cc5c4c3s2)cc1 ; c1ccc(-c2nc3ccc4ccc5ccc(-c6ccc7c(c6)C6(c8ccccc8-c8ccccc86)c6ccc8ccccc8c6-7)cc5c4c3s2)cc1 ; c1ccc(-c2nc3ccc4ccc5ccc(-c6ccc7c(c6)C(c6ccccc6)(c6ccccc6)c6cc8ccccc8cc6-7)cc5c4c3s2)cc1]

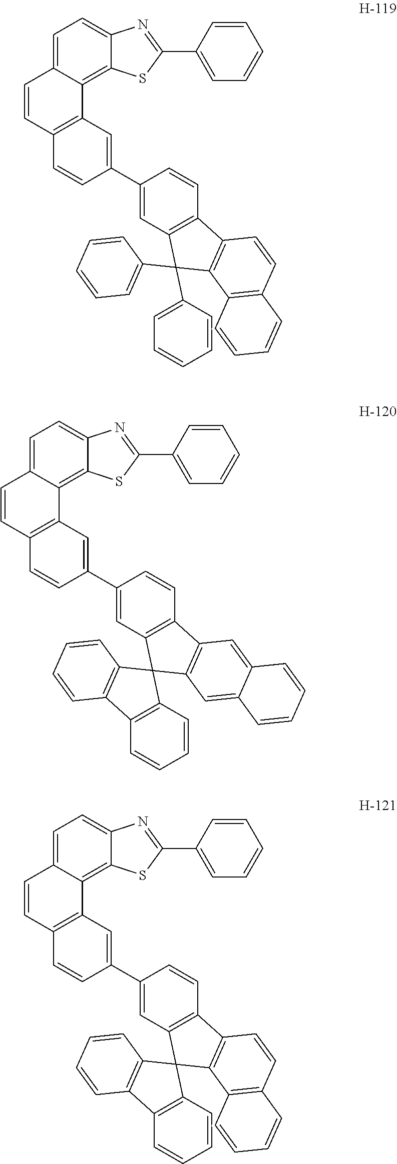 [SMILES: c1ccc(-c2nc3ccc4ccc5ccc(-c6ccc7c(c6)C(c6ccccc6)(c6ccccc6)c6c-7ccc7ccccc67)cc5c4c3s2)cc1 ; c1ccc(-c2nc3ccc4ccc5ccc(-c6ccc7c(c6)C6(c8ccccc8-c8ccccc86)c6cc8ccccc8cc6-7)cc5c4c3s2)cc1 ; c1ccc(-c2nc3ccc4ccc5ccc(-c6ccc7c(c6)C6(c8ccccc8-c8ccccc86)c6c-7ccc7ccccc67)cc5c4c3s2)cc1]

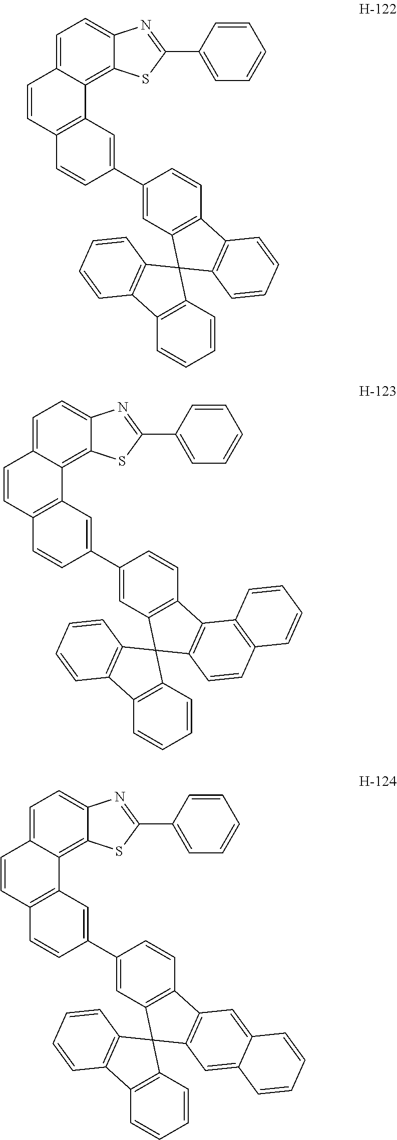

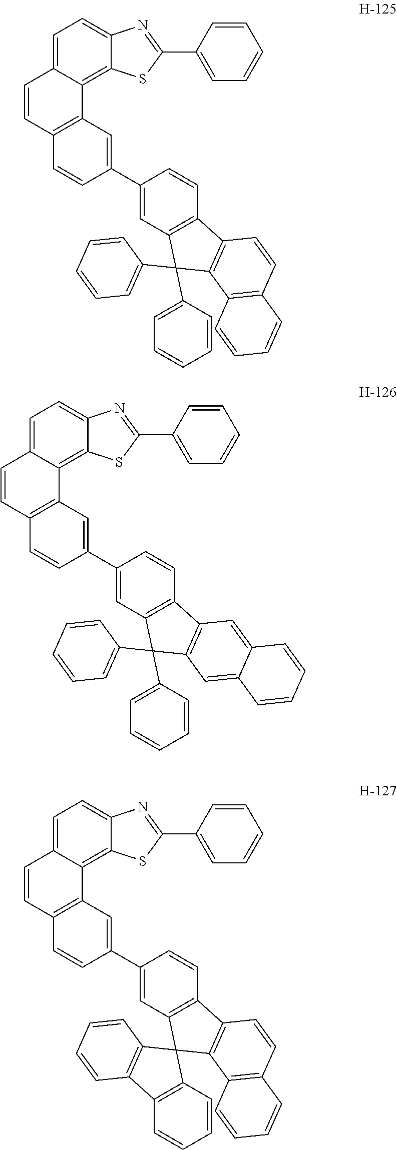

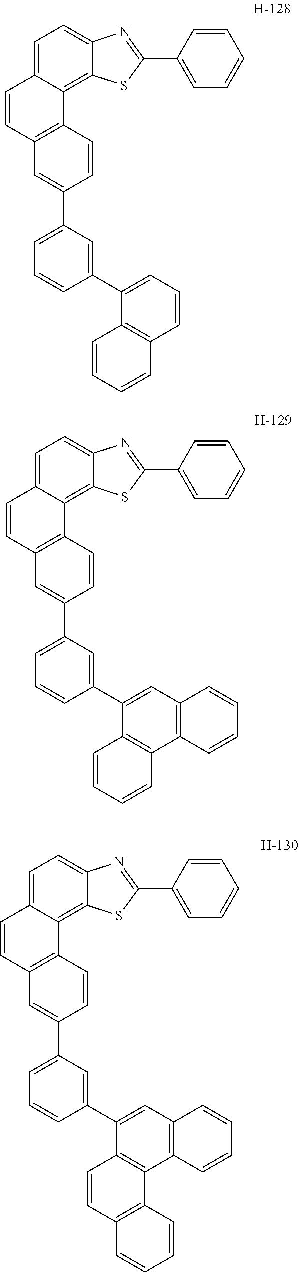 [SMILES: c1ccc(-c2nc3ccc4ccc5cc(-c6cccc(-c7cccc8ccccc78)c6)ccc5c4c3s2)cc1 ; c1ccc(-c2nc3ccc4ccc5cc(-c6cccc(-c7cc8ccccc8c8ccccc78)c6)ccc5c4c3s2)cc1 ; c1ccc(-c2nc3ccc4ccc5cc(-c6cccc(-c7cc8ccccc8c8c7ccc7ccccc78)c6)ccc5c4c3s2)cc1]

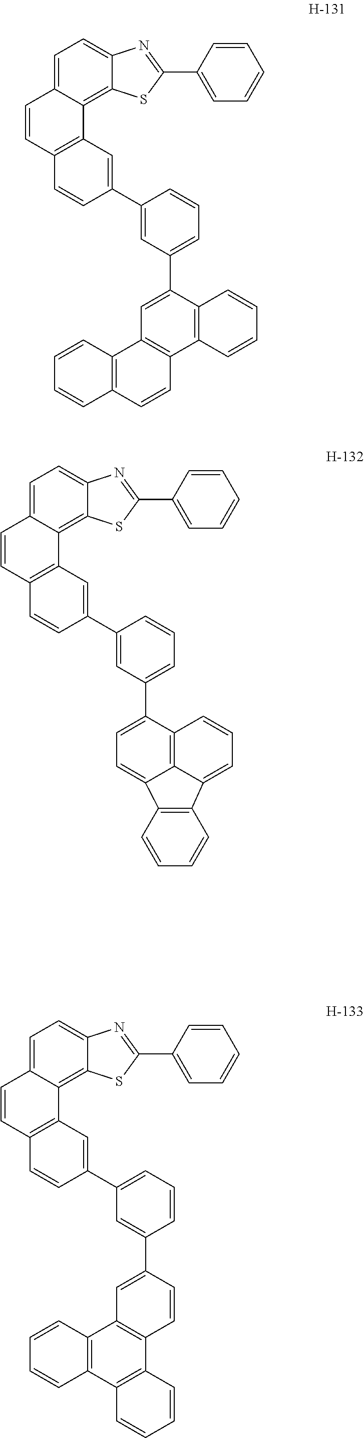 [SMILES: c1ccc(-c2nc3ccc4ccc5ccc(-c6cccc(-c7cc8c9ccccc9ccc8c8ccccc78)c6)cc5c4c3s2)cc1 ; c1ccc(-c2nc3ccc4ccc5ccc(-c6cccc(-c7ccc8c9c(cccc79)-c7ccccc7-8)c6)cc5c4c3s2)cc1 ; c1ccc(-c2nc3ccc4ccc5ccc(-c6cccc(-c7ccc8c9ccccc9c9ccccc9c8c7)c6)cc5c4c3s2)cc1]

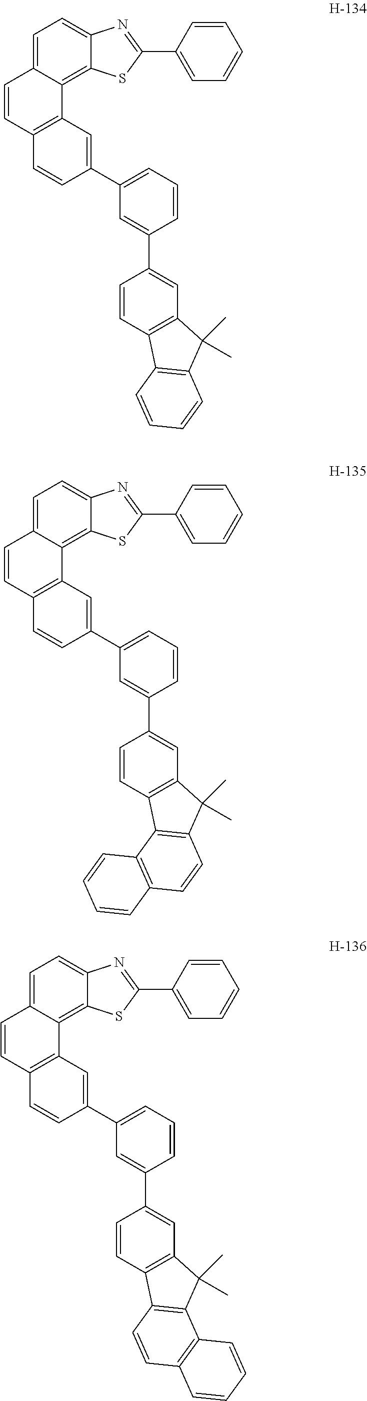 [SMILES: CC1(C)c2ccccc2-c2ccc(-c3cccc(-c4ccc5ccc6ccc7nc(-c8ccccc8)sc7c6c5c4)c3)cc21 ; CC1(C)c2cc(-c3cccc(-c4ccc5ccc6ccc7nc(-c8ccccc8)sc7c6c5c4)c3)ccc2-c2c1ccc1ccccc21 ; CC1(C)c2cc(-c3cccc(-c4ccc5ccc6ccc7nc(-c8ccccc8)sc7c6c5c4)c3)ccc2-c2ccc3ccccc3c21]

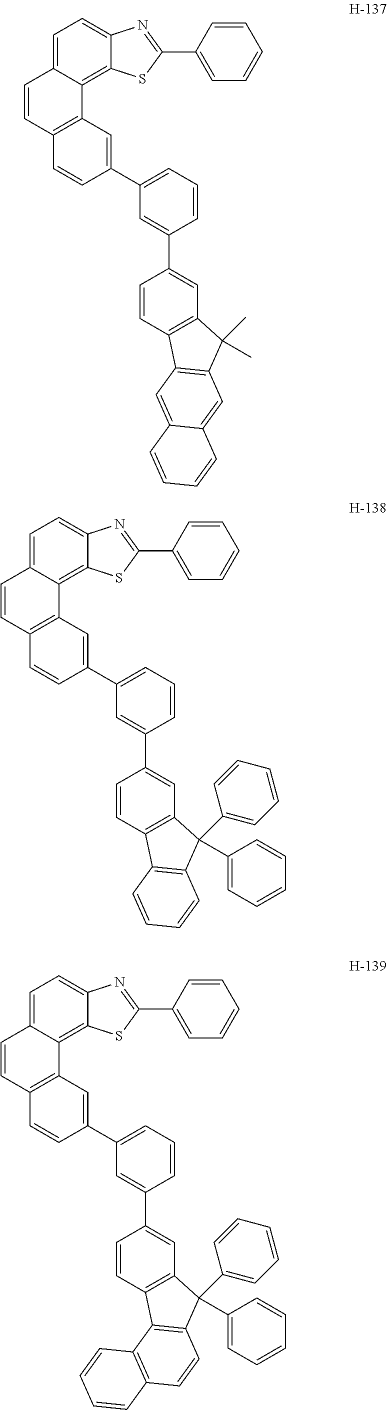

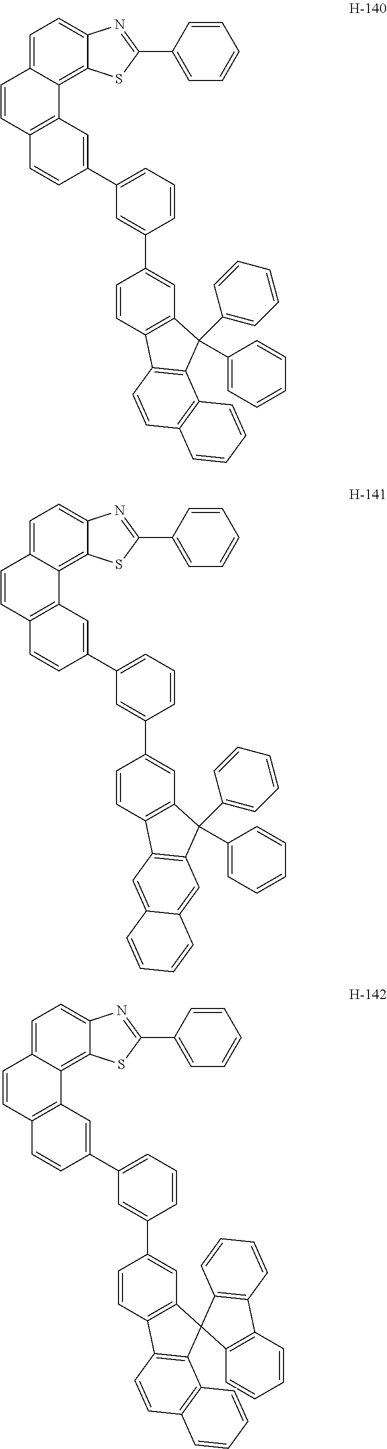

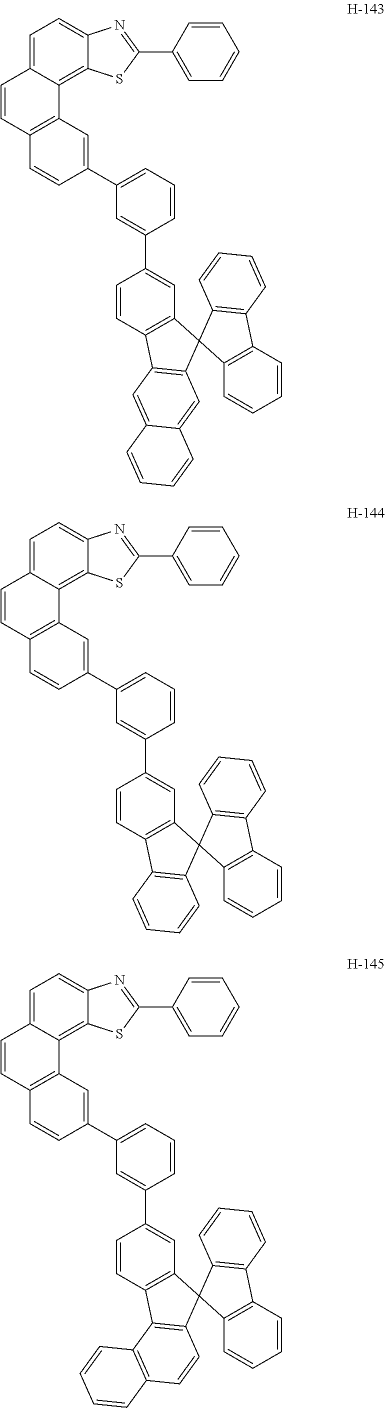

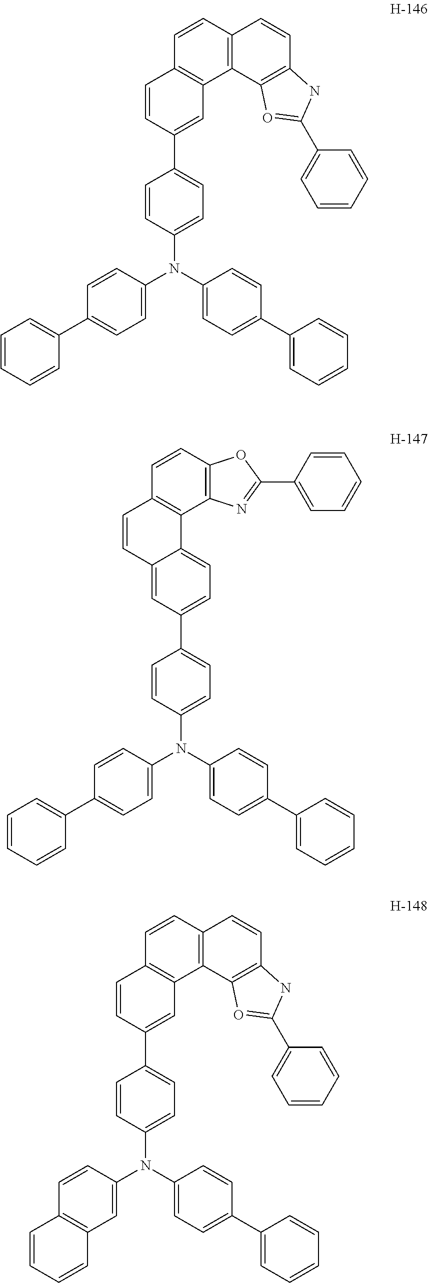

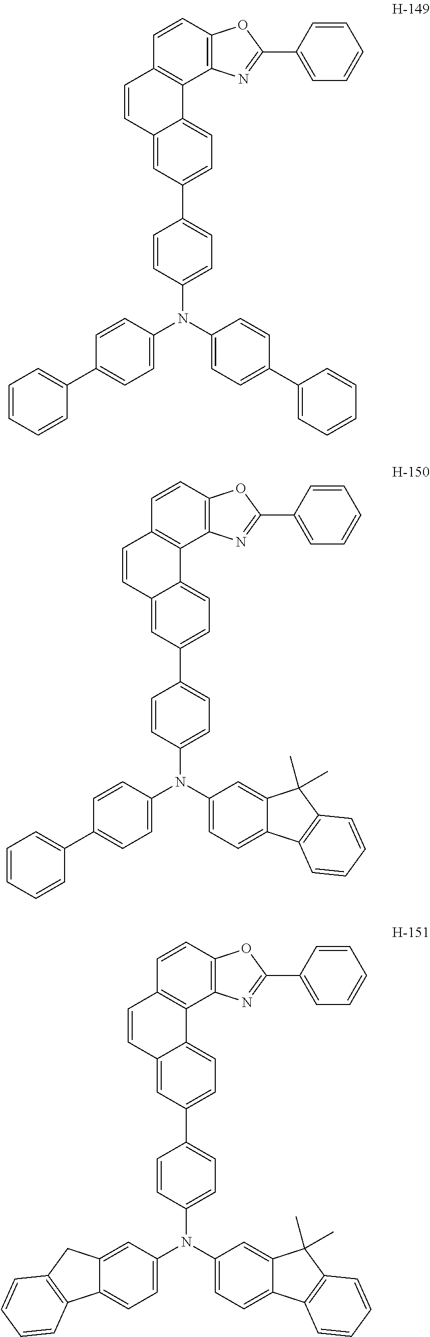 [SMILES: c1ccc(-c2ccc(N(c3ccc(-c4ccccc4)cc3)c3ccc(-c4ccc5c(ccc6ccc7oc(-c8ccccc8)nc7c65)c4)cc3)cc2)cc1 ; CC1(C)c2ccccc2-c2ccc(N(c3ccc(-c4ccccc4)cc3)c3ccc(-c4ccc5c(ccc6ccc7oc(-c8ccccc8)nc7c65)c4)cc3)cc21 ; CC1(C)c2ccccc2-c2ccc(N(c3ccc(-c4ccc5c(ccc6ccc7oc(-c8ccccc8)nc7c65)c4)cc3)c3ccc4c(c3)Cc3ccccc3-4)cc21]

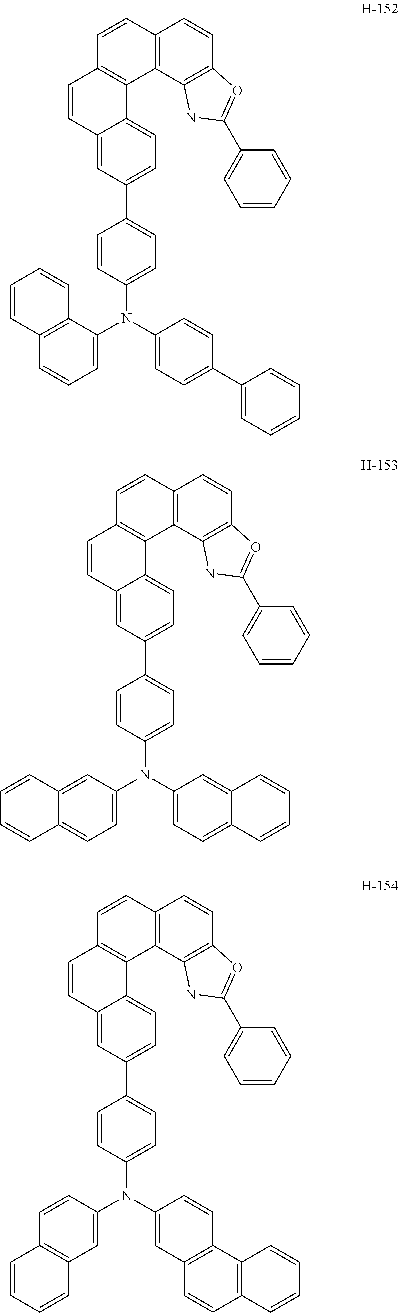

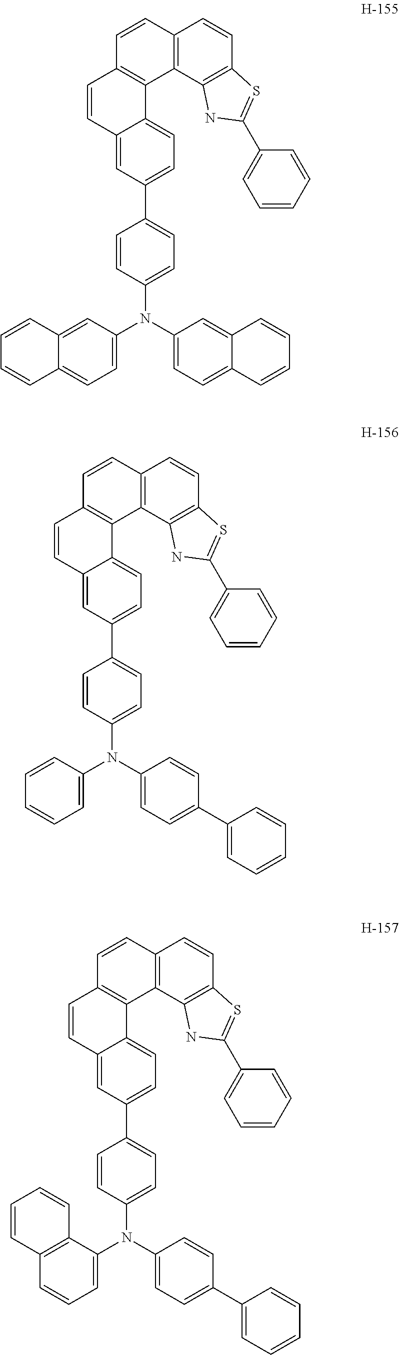

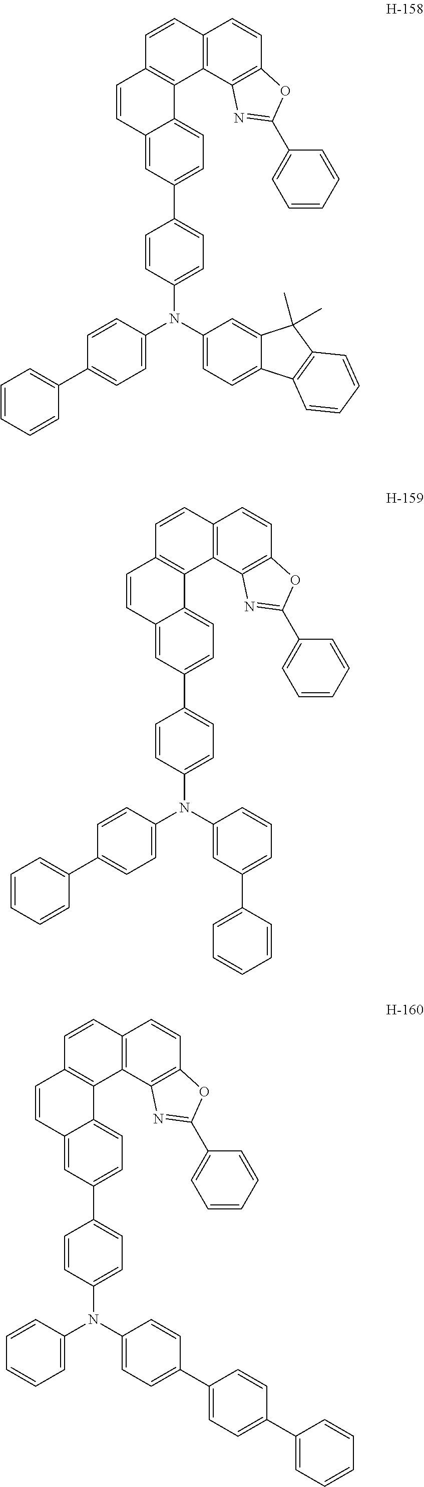 [SMILES: CC1(C)c2ccccc2-c2ccc(N(c3ccc(-c4ccccc4)cc3)c3ccc(-c4ccc5c(ccc6ccc7ccc8oc(-c9ccccc9)nc8c7c65)c4)cc3)cc21 ; c1ccc(-c2ccc(N(c3ccc(-c4ccc5c(ccc6ccc7ccc8oc(-c9ccccc9)nc8c7c65)c4)cc3)c3cccc(-c4ccccc4)c3)cc2)cc1 ; c1ccc(-c2ccc(-c3ccc(N(c4ccccc4)c4ccc(-c5ccc6c(ccc7ccc8ccc9oc(-c%10ccccc%10)nc9c8c76)c5)cc4)cc3)cc2)cc1]

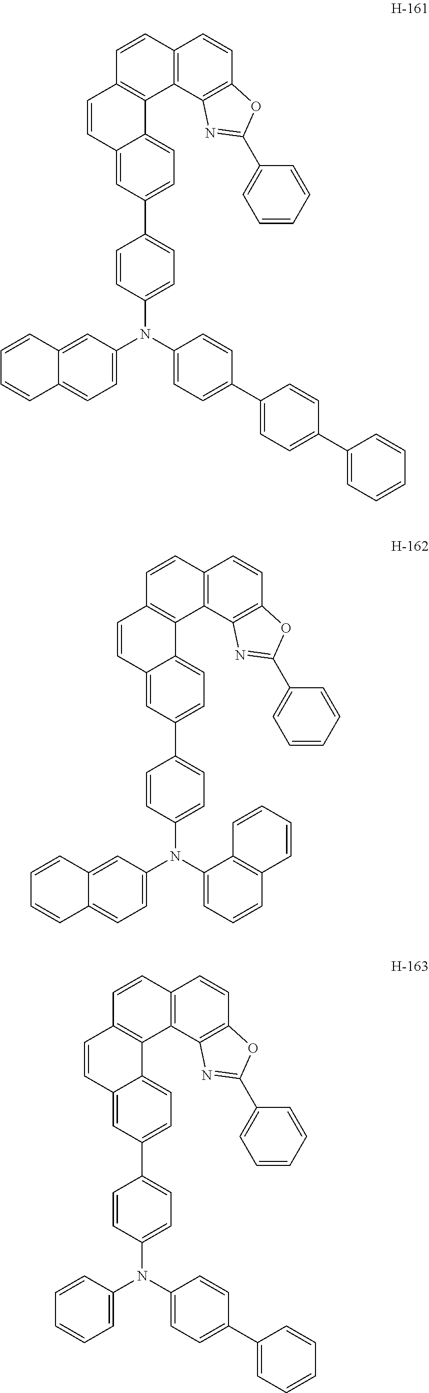 [SMILES: c1ccc(-c2ccc(-c3ccc(N(c4ccc(-c5ccc6c(ccc7ccc8ccc9oc(-c%10ccccc%10)nc9c8c76)c5)cc4)c4ccc5ccccc5c4)cc3)cc2)cc1 ; c1ccc(-c2nc3c(ccc4ccc5ccc6cc(-c7ccc(N(c8ccc9ccccc9c8)c8cccc9ccccc89)cc7)ccc6c5c43)o2)cc1 ; c1ccc(-c2ccc(N(c3ccccc3)c3ccc(-c4ccc5c(ccc6ccc7ccc8oc(-c9ccccc9)nc8c7c65)c4)cc3)cc2)cc1]

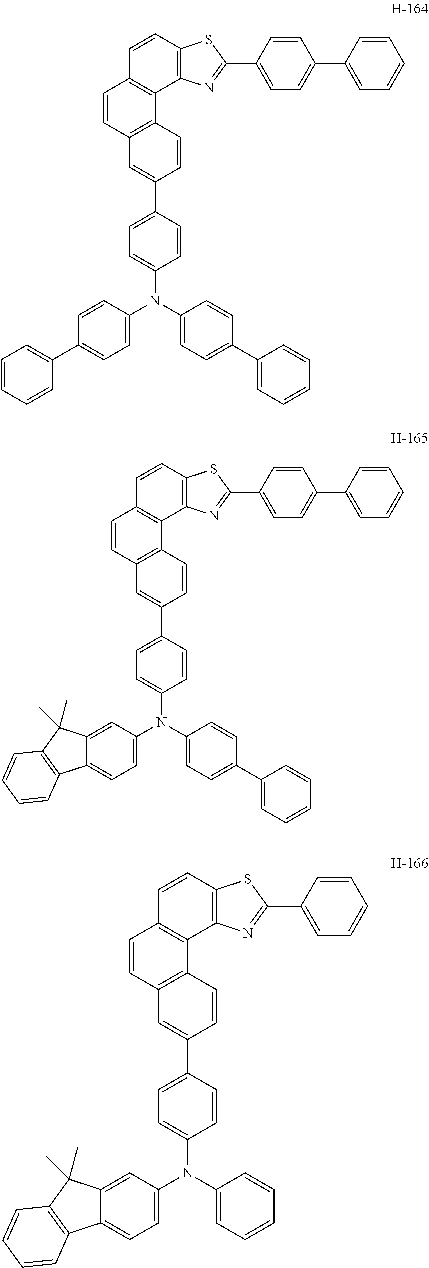 [SMILES: c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21 ; CC1(C)c2ccccc2-c2ccc(N(c3ccccc3)c3ccc(-c4ccc5c(ccc6ccc7sc(-c8ccccc8)nc7c65)c4)cc3)cc21]

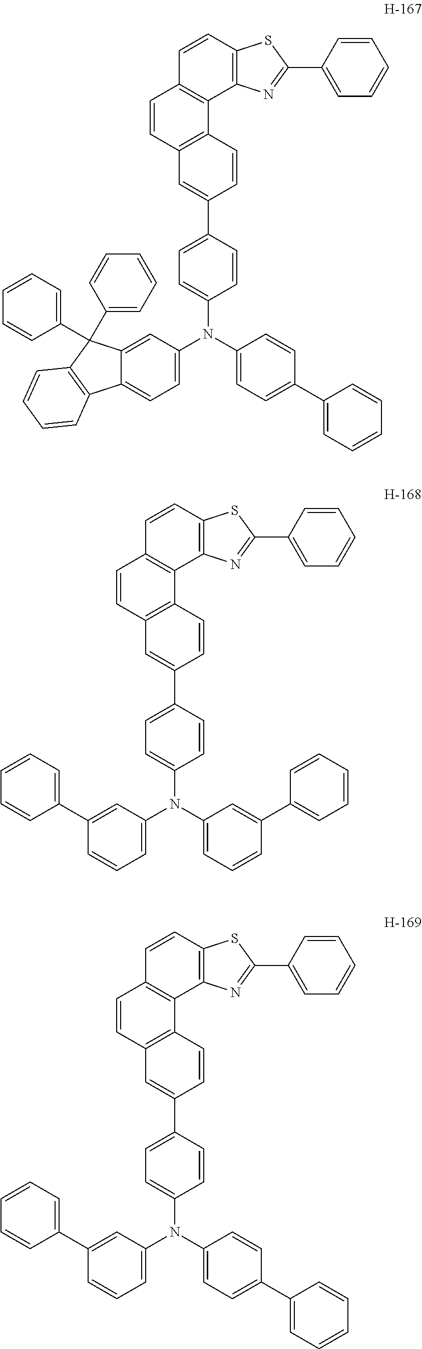 [SMILES: c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1 ; c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3cccc(-c4ccccc4)c3)cc2)cc1]

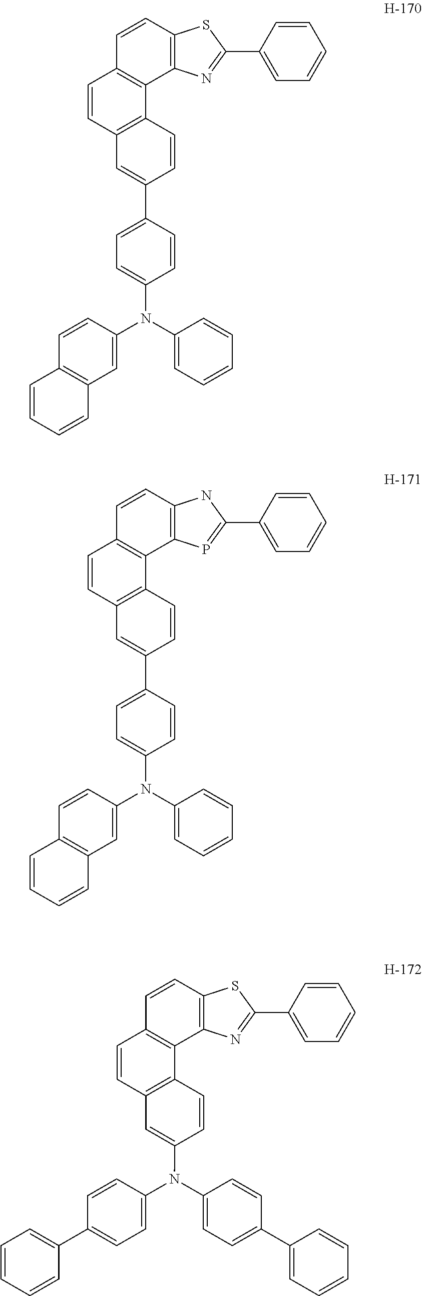 [SMILES: c1ccc(-c2nc3c(ccc4ccc5cc(-c6ccc(N(c7ccccc7)c7ccc8ccccc8c7)cc6)ccc5c43)s2)cc1 ; c1ccc(-c2[nH]c3ccc4ccc5cc(-c6ccc(N(c7ccccc7)c7ccc8ccccc8c7)cc6)ccc5c4c3p2)cc1 ; c1ccc(-c2ccc(N(c3ccc(-c4ccccc4)cc3)c3ccc4c(ccc5ccc6sc(-c7ccccc7)nc6c54)c3)cc2)cc1]

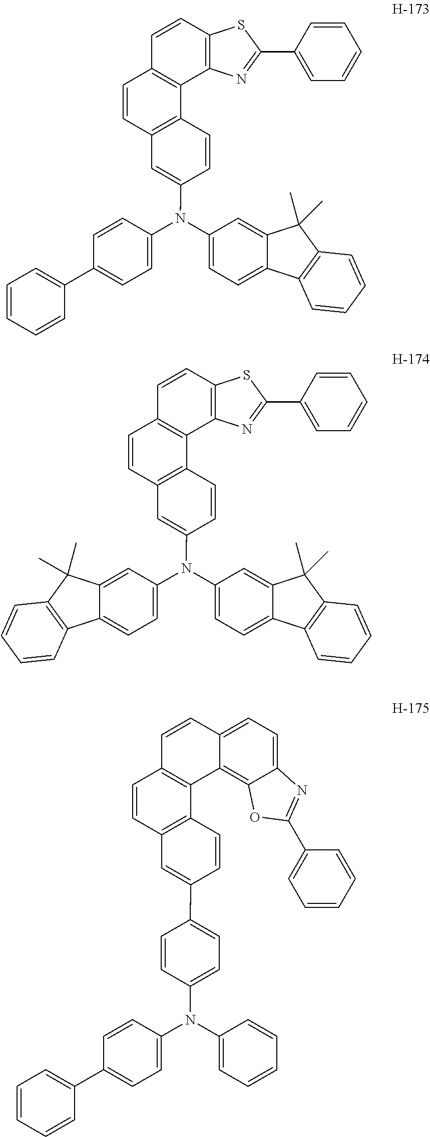 [SMILES: CC1(C)c2ccccc2-c2ccc(N(c3ccc(-c4ccccc4)cc3)c3ccc4c(ccc5ccc6sc(-c7ccccc7)nc6c54)c3)cc21 ; CC1(C)c2ccccc2-c2ccc(N(c3ccc4c(c3)C(C)(C)c3ccccc3-4)c3ccc4c(ccc5ccc6sc(-c7ccccc7)nc6c54)c3)cc21 ; c1ccc(-c2ccc(N(c3ccccc3)c3ccc(-c4ccc5c(ccc6ccc7ccc8nc(-c9ccccc9)oc8c7c65)c4)cc3)cc2)cc1]

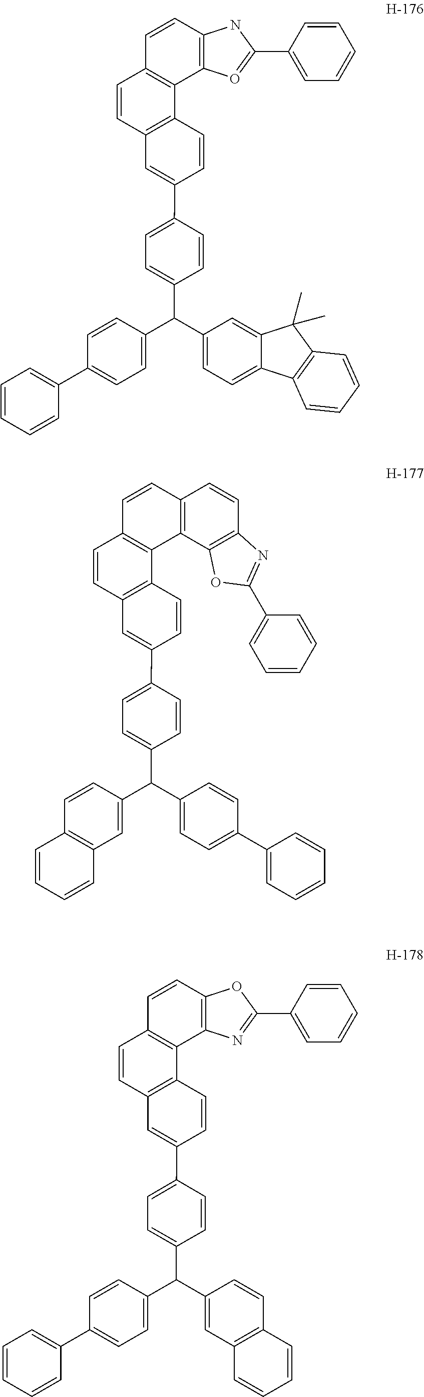

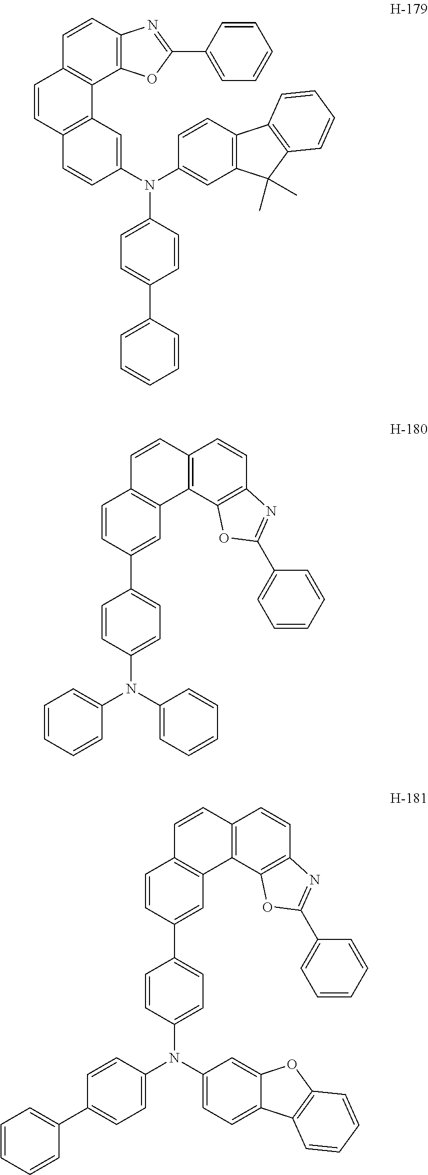 [SMILES: CC1(C)c2ccccc2-c2ccc(N(c3ccc(-c4ccccc4)cc3)c3ccc4ccc5ccc6nc(-c7ccccc7)oc6c5c4c3)cc21 ; c1ccc(-c2nc3ccc4ccc5ccc(-c6ccc(N(c7ccccc7)c7ccccc7)cc6)cc5c4c3o2)cc1 ; c1ccc(-c2ccc(N(c3ccc(-c4ccc5ccc6ccc7nc(-c8ccccc8)oc7c6c5c4)cc3)c3ccc4c(c3)oc3ccccc34)cc2)cc1]

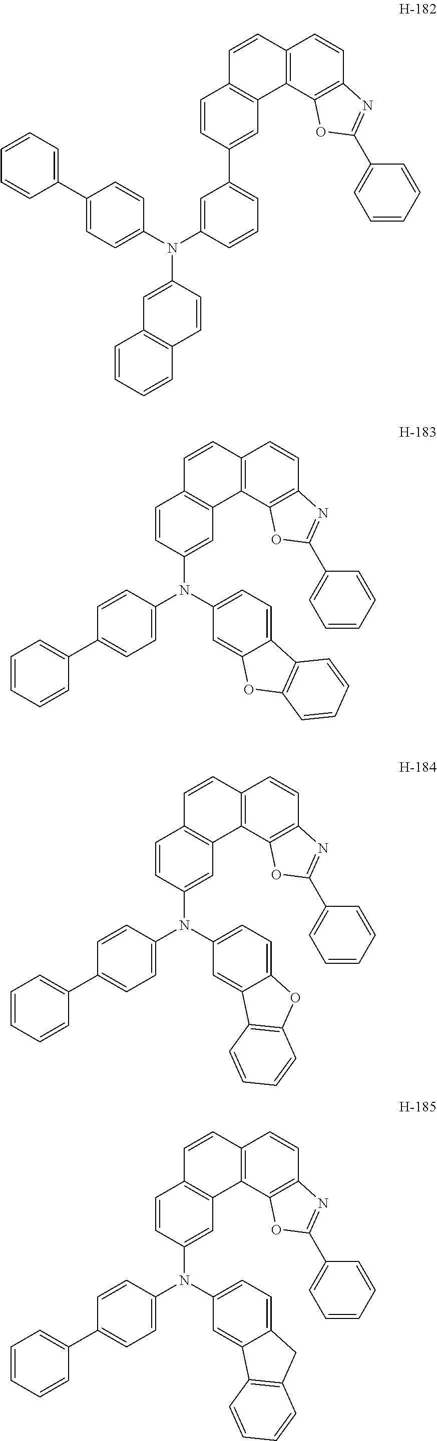 [SMILES: c1ccc(-c2ccc(N(c3cccc(-c4ccc5ccc6ccc7nc(-c8ccccc8)oc7c6c5c4)c3)c3ccc4ccccc4c3)cc2)cc1 ; c1ccc(-c2ccc(N(c3ccc4c(c3)oc3ccccc34)c3ccc4ccc5ccc6nc(-c7ccccc7)oc6c5c4c3)cc2)cc1 ; c1ccc(-c2ccc(N(c3ccc4oc5ccccc5c4c3)c3ccc4ccc5ccc6nc(-c7ccccc7)oc6c5c4c3)cc2)cc1 ; c1ccc(-c2ccc(N(c3ccc4c(c3)-c3ccccc3C4)c3ccc4ccc5ccc6nc(-c7ccccc7)oc6c5c4c3)cc2)cc1]

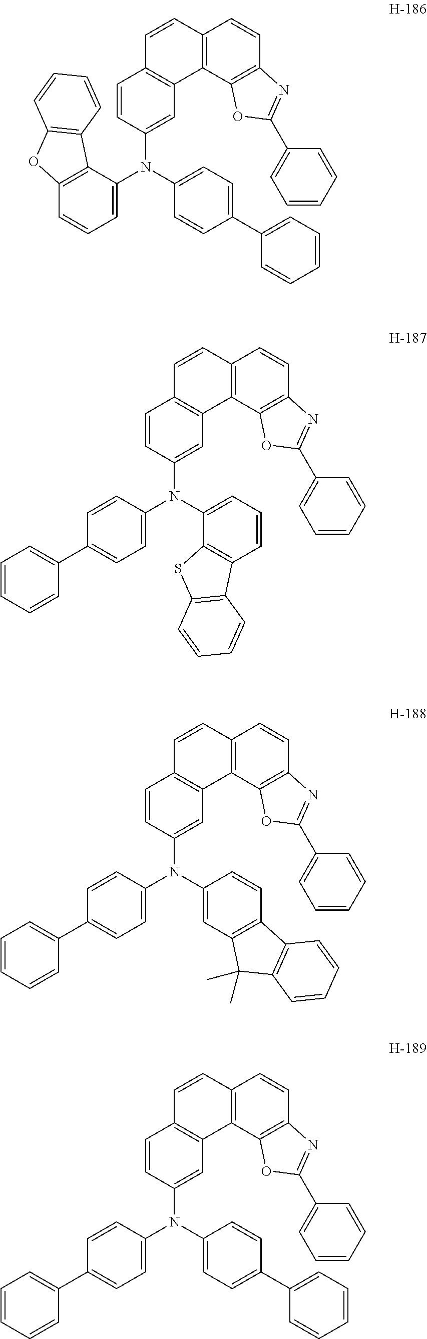 [SMILES: c1ccc(-c2ccc(N(c3ccc4ccc5ccc6nc(-c7ccccc7)oc6c5c4c3)c3cccc4oc5ccccc5c34)cc2)cc1 ; c1ccc(-c2ccc(N(c3ccc4ccc5ccc6nc(-c7ccccc7)oc6c5c4c3)c3cccc4c3sc3ccccc34)cc2)cc1 ; CC1(C)c2ccccc2-c2ccc(N(c3ccc(-c4ccccc4)cc3)c3ccc4ccc5ccc6nc(-c7ccccc7)oc6c5c4c3)cc21 ; c1ccc(-c2ccc(N(c3ccc(-c4ccccc4)cc3)c3ccc4ccc5ccc6nc(-c7ccccc7)oc6c5c4c3)cc2)cc1]

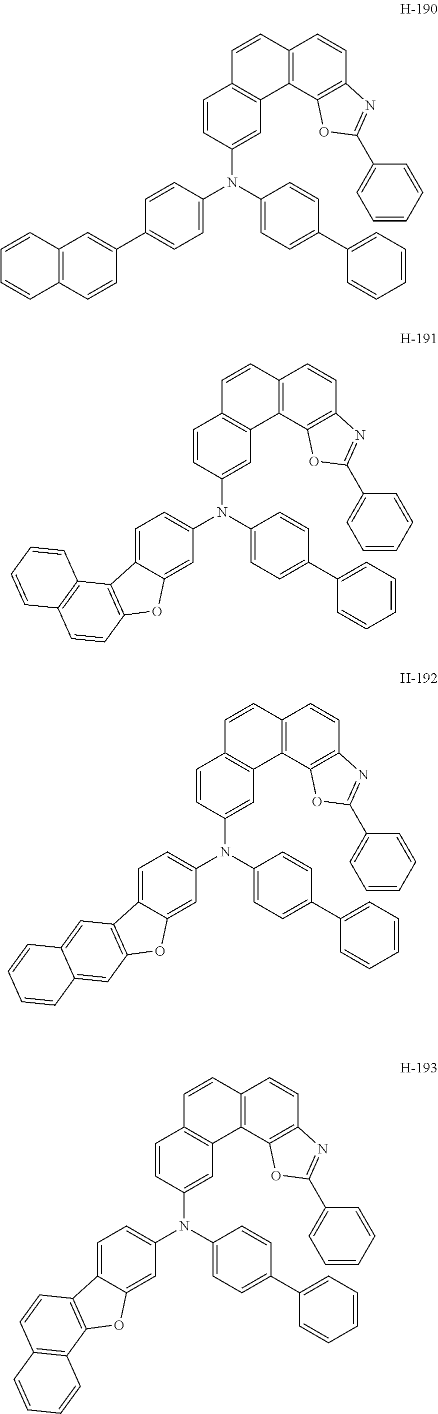 [SMILES: c1ccc(-c2ccc(N(c3ccc(-c4ccc5ccccc5c4)cc3)c3ccc4ccc5ccc6nc(-c7ccccc7)oc6c5c4c3)cc2)cc1 ; c1ccc(-c2ccc(N(c3ccc4c(c3)oc3ccc5ccccc5c34)c3ccc4ccc5ccc6nc(-c7ccccc7)oc6c5c4c3)cc2)cc1 ; c1ccc(-c2ccc(N(c3ccc4c(c3)oc3cc5ccccc5cc34)c3ccc4ccc5ccc6nc(-c7ccccc7)oc6c5c4c3)cc2)cc1 ; c1ccc(-c2ccc(N(c3ccc4c(c3)oc3c5ccccc5ccc43)c3ccc4ccc5ccc6nc(-c7ccccc7)oc6c5c4c3)cc2)cc1]

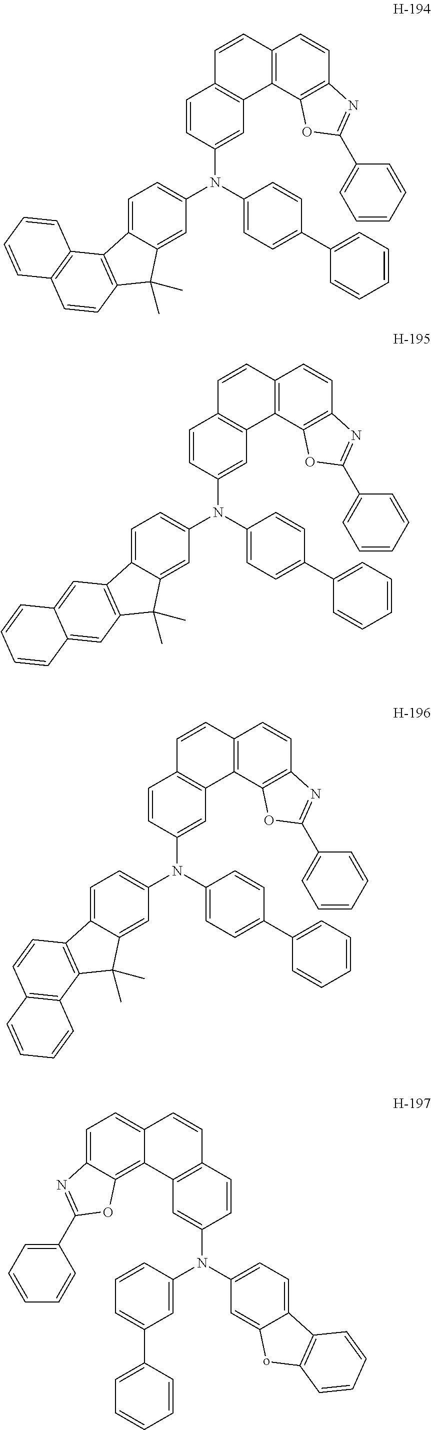

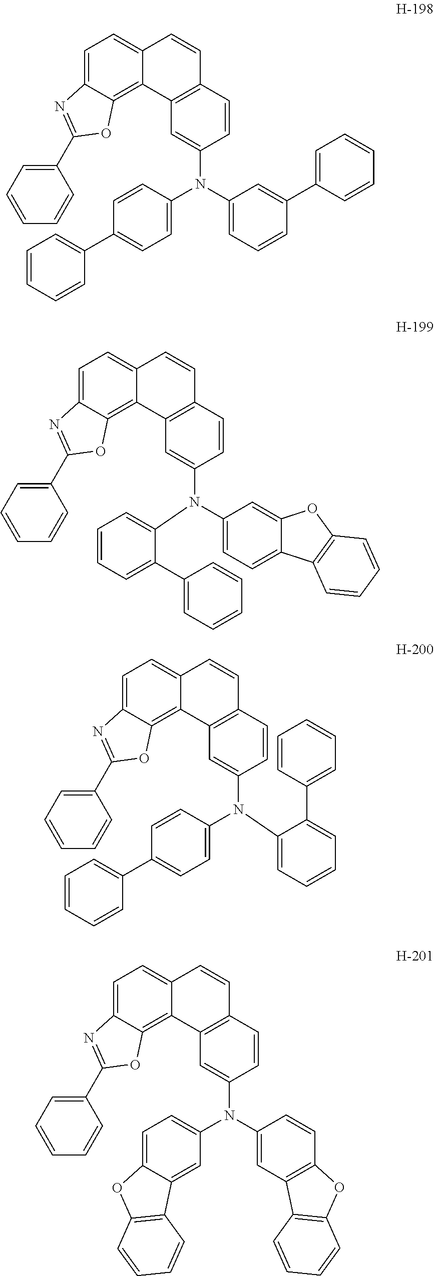

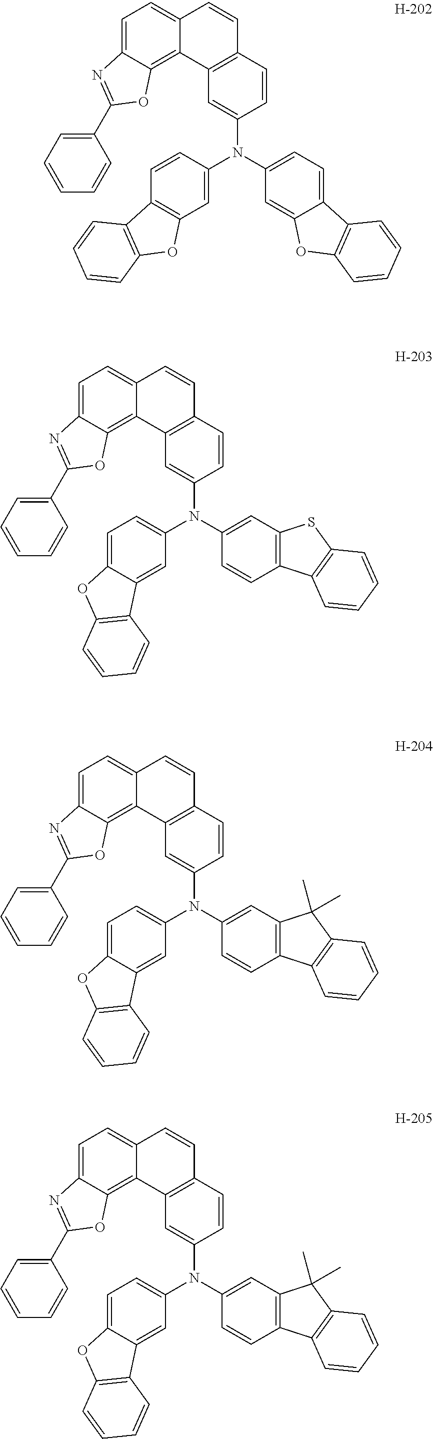 [SMILES: c1ccc(-c2nc3ccc4ccc5ccc(N(c6ccc7c(c6)oc6ccccc67)c6ccc7c(c6)oc6ccccc67)cc5c4c3o2)cc1 ; c1ccc(-c2nc3ccc4ccc5ccc(N(c6ccc7c(c6)sc6ccccc67)c6ccc7oc8ccccc8c7c6)cc5c4c3o2)cc1 ; CC1(C)c2ccccc2-c2ccc(N(c3ccc4oc5ccccc5c4c3)c3ccc4ccc5ccc6nc(-c7ccccc7)oc6c5c4c3)cc21 ; CC1(C)c2ccccc2-c2ccc(N(c3ccc4oc5ccccc5c4c3)c3ccc4ccc5ccc6nc(-c7ccccc7)oc6c5c4c3)cc21]

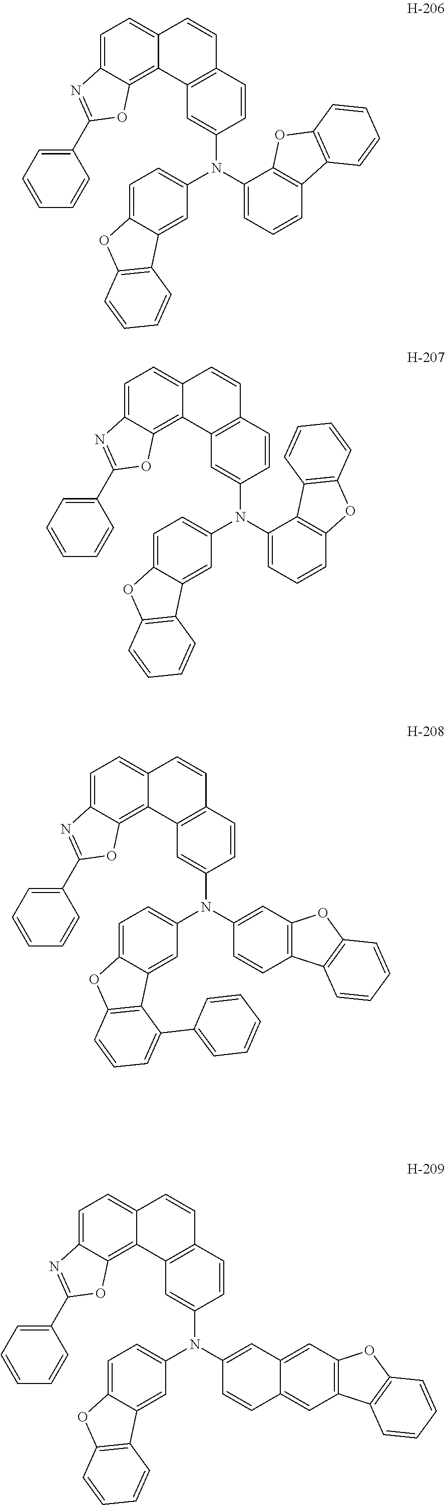

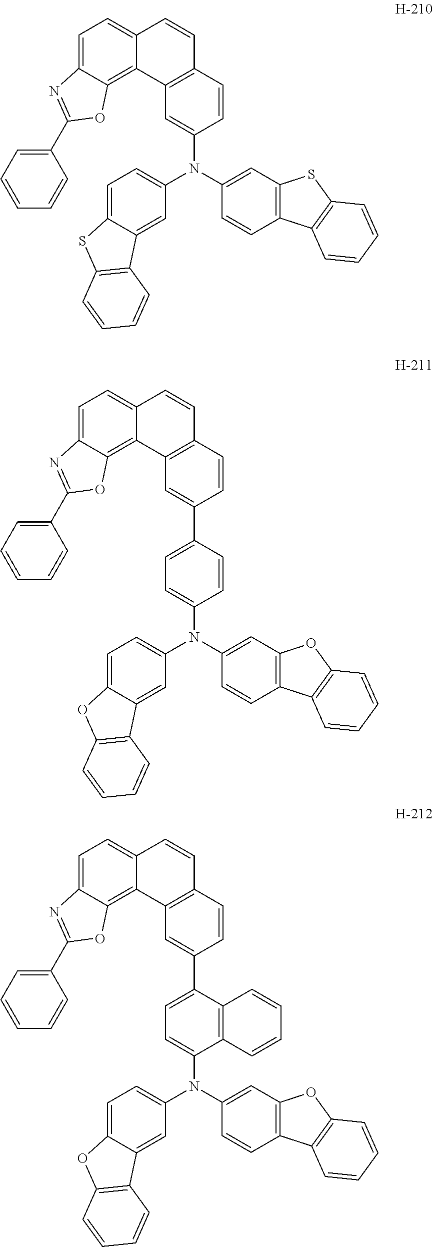

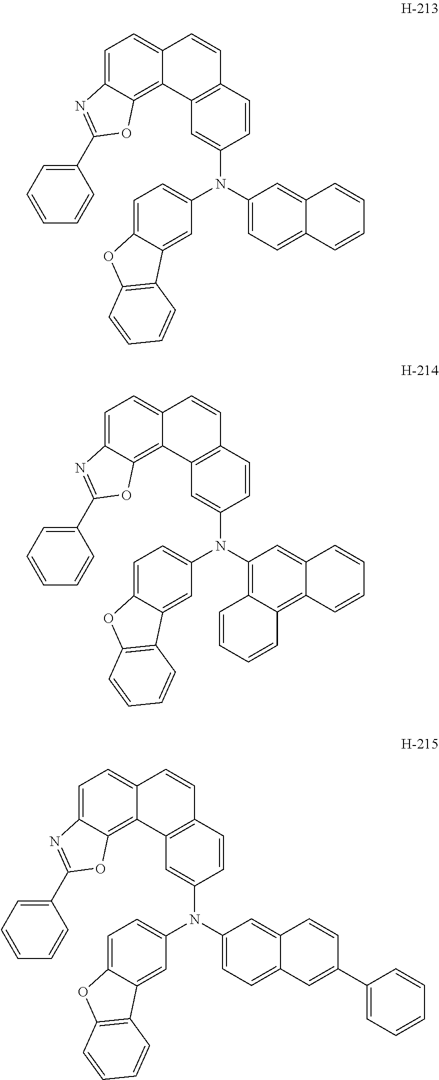 [SMILES: c1ccc(-c2nc3ccc4ccc5ccc(N(c6ccc7ccccc7c6)c6ccc7oc8ccccc8c7c6)cc5c4c3o2)cc1 ; c1ccc(-c2nc3ccc4ccc5ccc(N(c6ccc7oc8ccccc8c7c6)c6cc7ccccc7c7ccccc67)cc5c4c3o2)cc1 ; c1ccc(-c2ccc3cc(N(c4ccc5oc6ccccc6c5c4)c4ccc5ccc6ccc7nc(-c8ccccc8)oc7c6c5c4)ccc3c2)cc1]

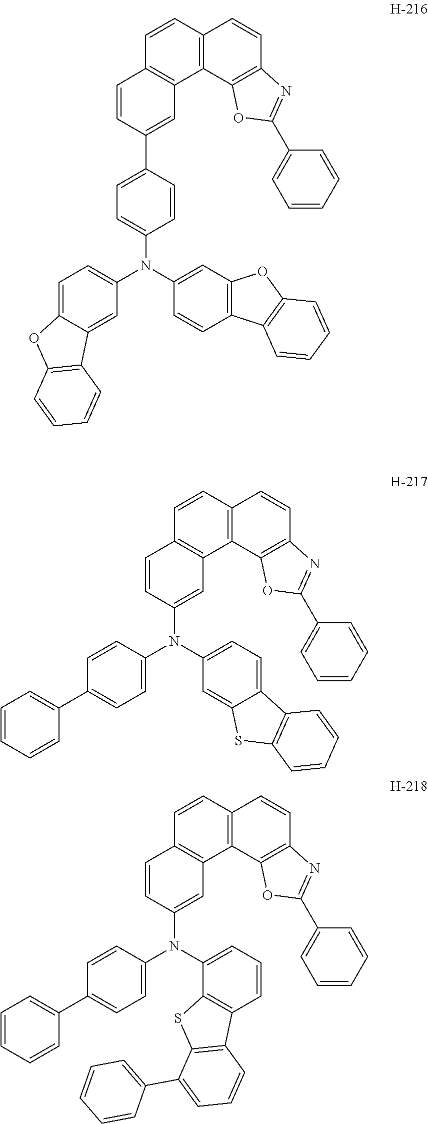

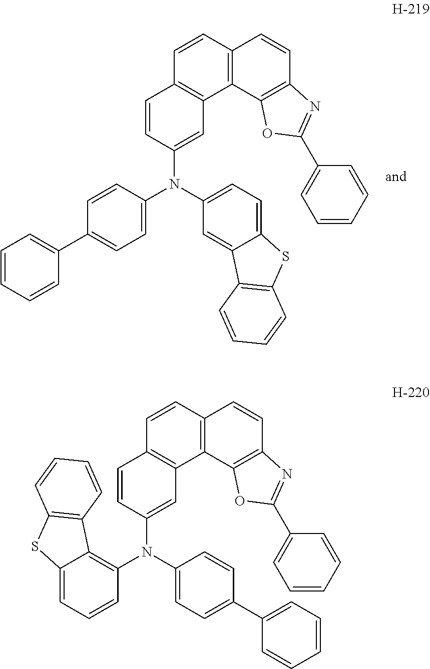 [SMILES: c1ccc(-c2ccc(N(c3ccc4sc5ccccc5c4c3)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3cccc4sc5ccccc5c34)cc2)cc1]

9. The plurality of host materials according to claim 1, wherein the compound represented by formula 3 is at least one selected from the following compounds: 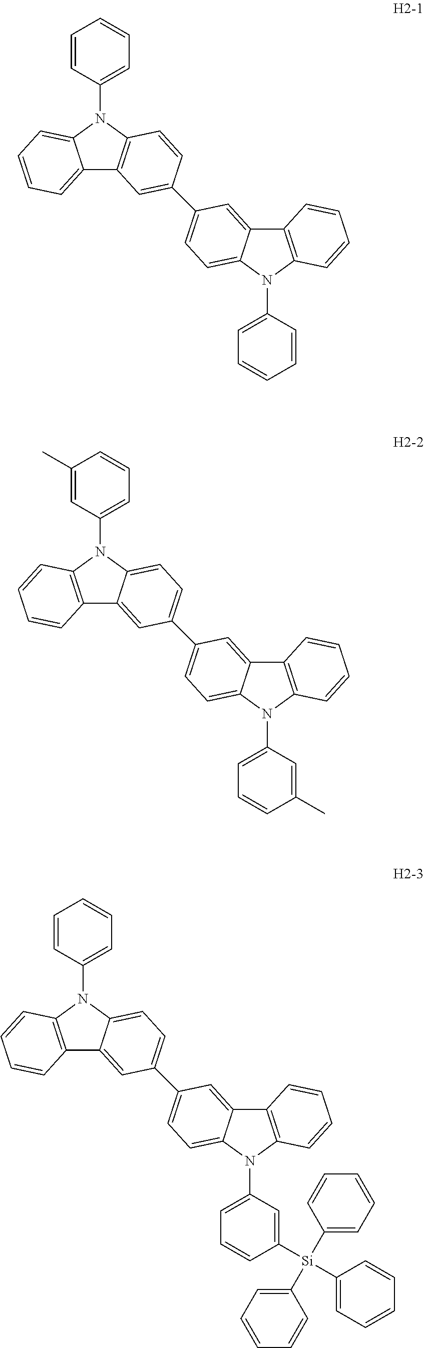 [SMILES: c1ccc(-n2c3ccccc3c3cc(-c4ccc5c(c4)c4ccccc4n5-c4ccccc4)ccc32)cc1 ; Cc1cccc(-n2c3ccccc3c3cc(-c4ccc5c(c4)c4ccccc4n5-c4cccc(C)c4)ccc32)c1 ; c1ccc(-n2c3ccccc3c3cc(-c4ccc5c(c4)c4ccccc4n5-c4cccc([Si](c5ccccc5)(c5ccccc5)c5ccccc5)c4)ccc32)cc1] 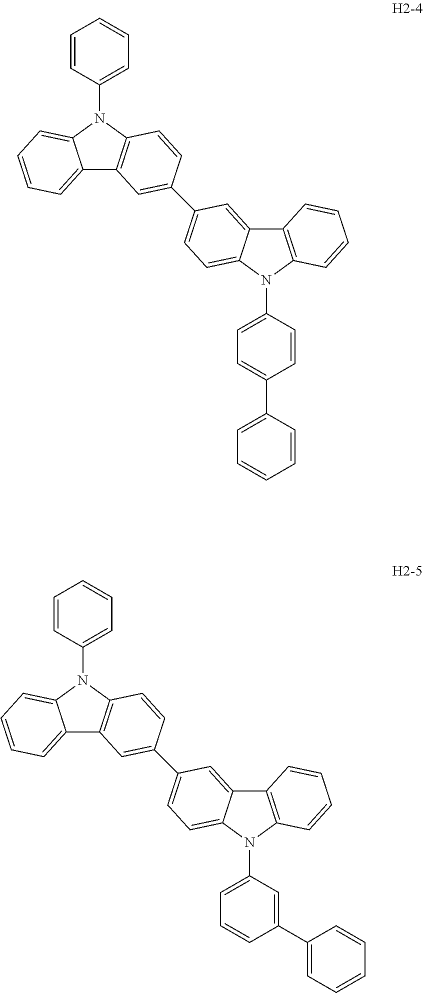 [SMILES: c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1] 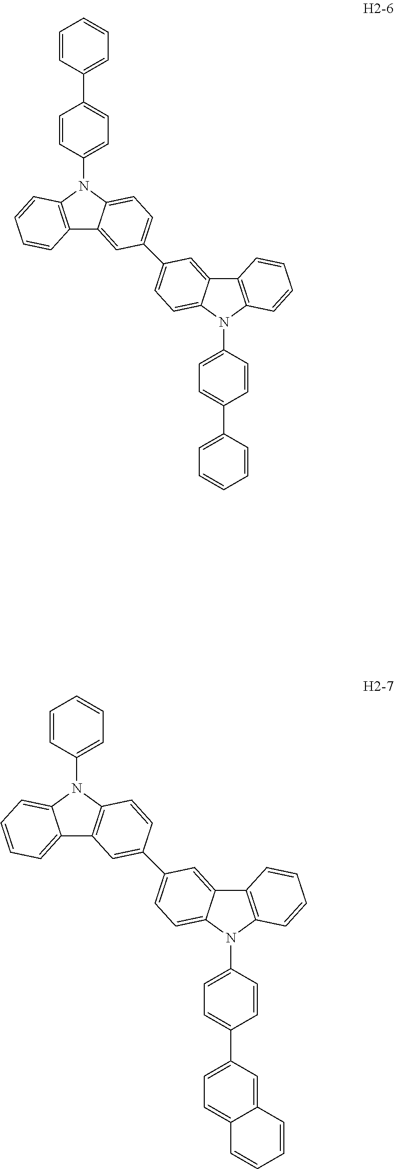 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-n2c3ccccc3c3cc(-c4ccc5c(c4)c4ccccc4n5-c4ccc(-c5ccc6ccccc6c5)cc4)ccc32)cc1] 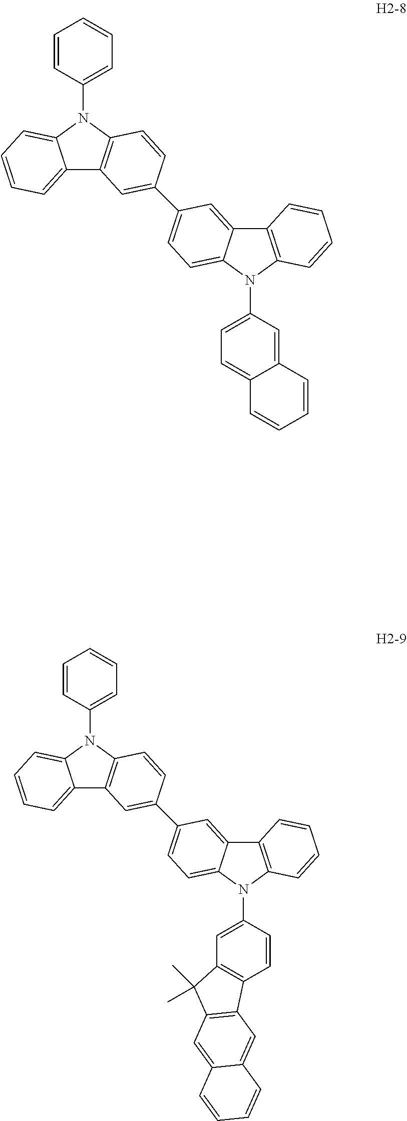 [SMILES: c1ccc(-n2c3ccccc3c3cc(-c4ccc5c(c4)c4ccccc4n5-c4ccc5ccccc5c4)ccc32)cc1 ; CC1(C)c2cc(-n3c4ccccc4c4cc(-c5ccc6c(c5)c5ccccc5n6-c5ccccc5)ccc43)ccc2-c2cc3ccccc3cc21] 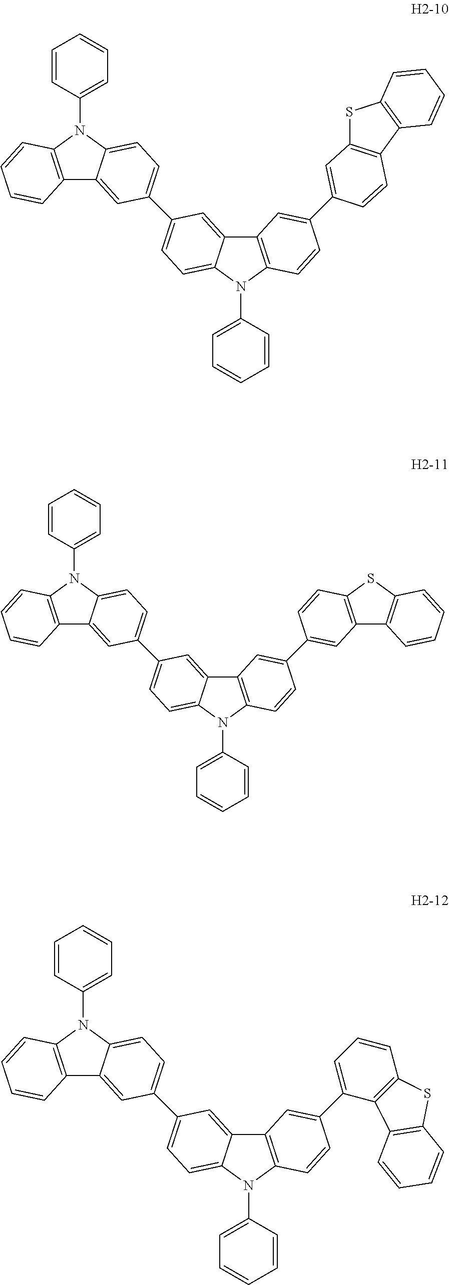 [SMILES: c1ccc(-n2c3ccccc3c3cc(-c4ccc5c(c4)c4cc(-c6ccc7c(c6)sc6ccccc67)ccc4n5-c4ccccc4)ccc32)cc1 ; c1ccc(-n2c3ccccc3c3cc(-c4ccc5c(c4)c4cc(-c6ccc7sc8ccccc8c7c6)ccc4n5-c4ccccc4)ccc32)cc1 ; c1ccc(-n2c3ccccc3c3cc(-c4ccc5c(c4)c4cc(-c6cccc7sc8ccccc8c67)ccc4n5-c4ccccc4)ccc32)cc1] 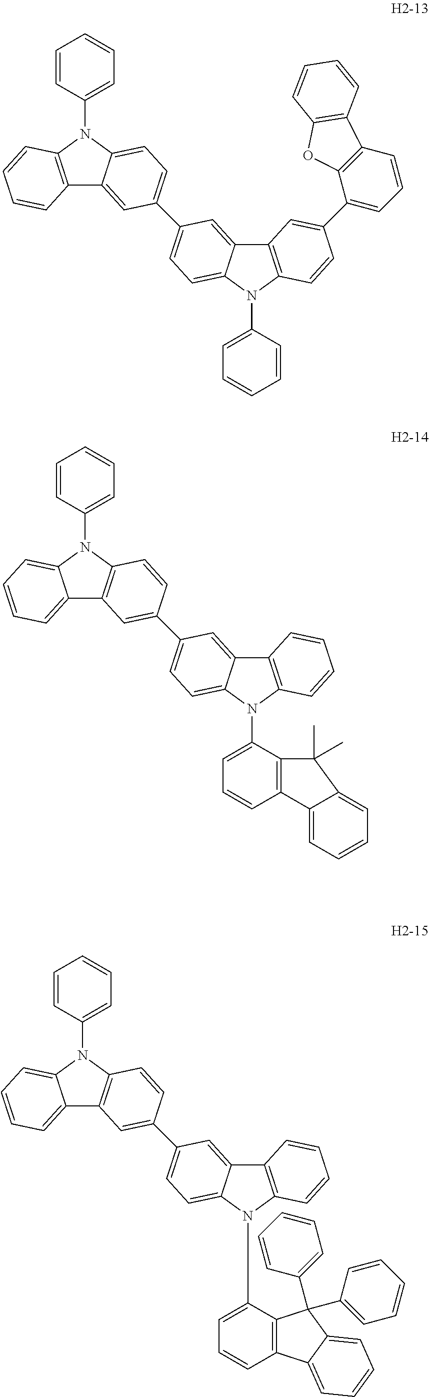 [SMILES: c1ccc(-n2c3ccccc3c3cc(-c4ccc5c(c4)c4cc(-c6cccc7c6oc6ccccc67)ccc4n5-c4ccccc4)ccc32)cc1 ; CC1(C)c2ccccc2-c2cccc(-n3c4ccccc4c4cc(-c5ccc6c(c5)c5ccccc5n6-c5ccccc5)ccc43)c21 ; c1ccc(-n2c3ccccc3c3cc(-c4ccc5c(c4)c4ccccc4n5-c4cccc5c4C(c4ccccc4)(c4ccccc4)c4ccccc4-5)ccc32)cc1] 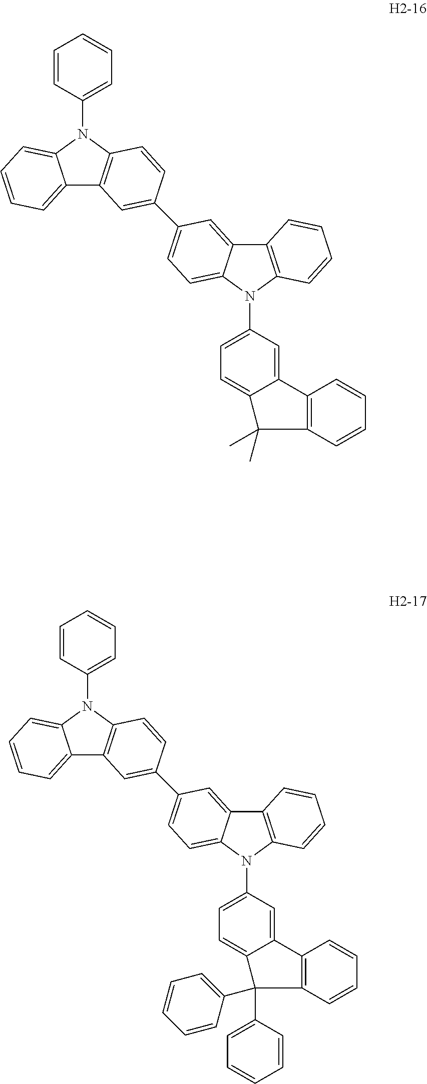 [SMILES: CC1(C)c2ccccc2-c2cc(-n3c4ccccc4c4cc(-c5ccc6c(c5)c5ccccc5n6-c5ccccc5)ccc43)ccc21 ; c1ccc(-n2c3ccccc3c3cc(-c4ccc5c(c4)c4ccccc4n5-c4ccc5c(c4)-c4ccccc4C5(c4ccccc4)c4ccccc4)ccc32)cc1] 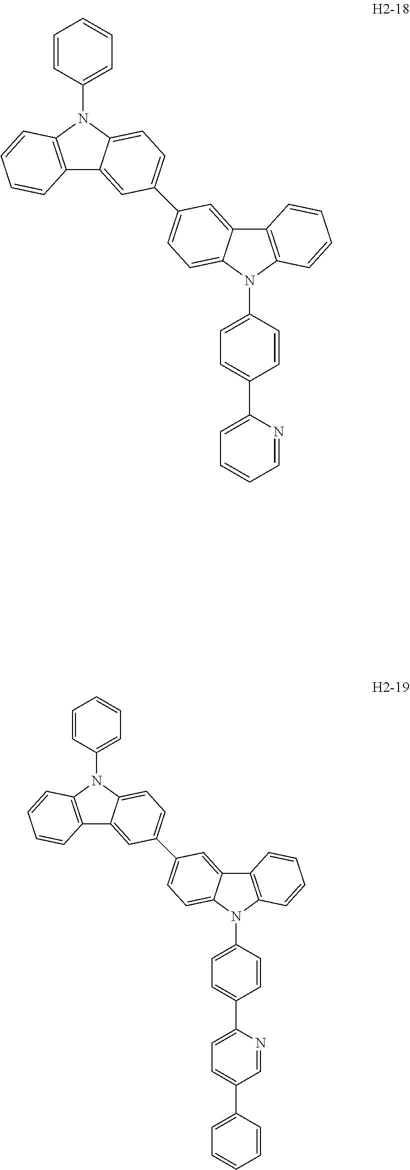 [SMILES: c1ccc(-n2c3ccccc3c3cc(-c4ccc5c(c4)c4ccccc4n5-c4ccc(-c5ccccn5)cc4)ccc32)cc1 ; c1ccc(-c2ccc(-c3ccc(-n4c5ccccc5c5cc(-c6ccc7c(c6)c6ccccc6n7-c6ccccc6)ccc54)cc3)nc2)cc1] 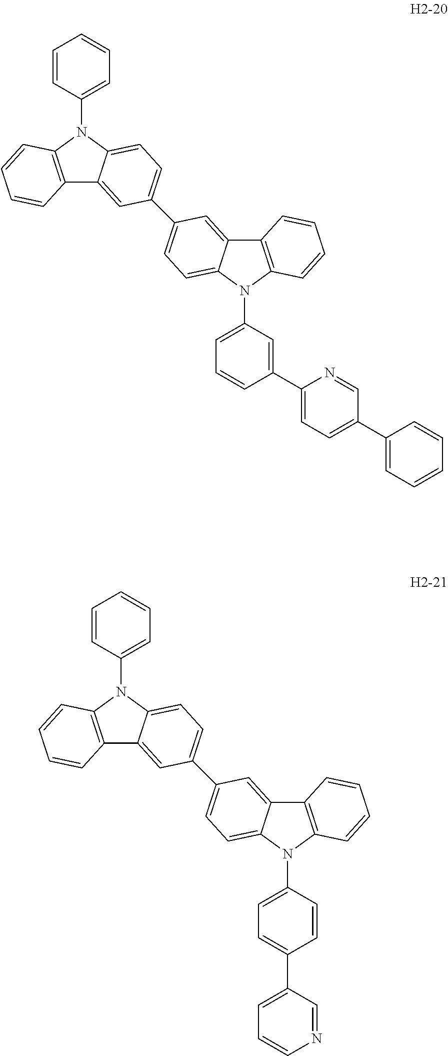 [SMILES: c1ccc(-c2ccc(-c3cccc(-n4c5ccccc5c5cc(-c6ccc7c(c6)c6ccccc6n7-c6ccccc6)ccc54)c3)nc2)cc1 ; c1ccc(-n2c3ccccc3c3cc(-c4ccc5c(c4)c4ccccc4n5-c4ccc(-c5cccnc5)cc4)ccc32)cc1] 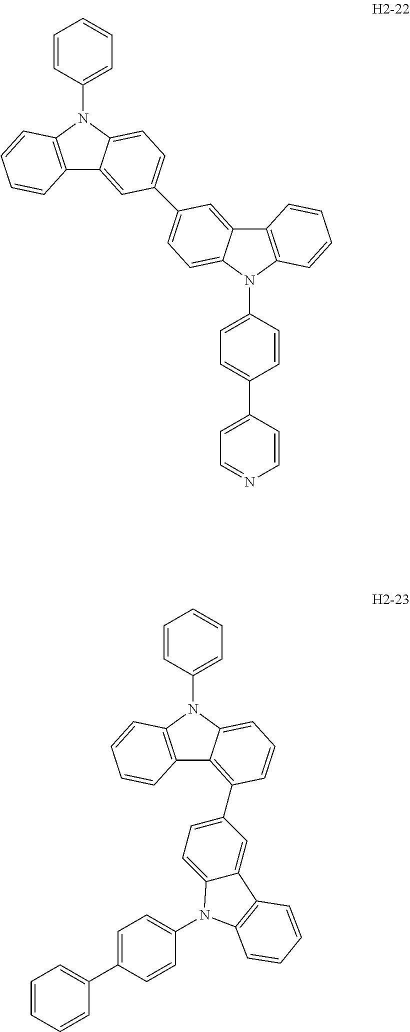 [SMILES: c1ccc(-n2c3ccccc3c3cc(-c4ccc5c(c4)c4ccccc4n5-c4ccc(-c5ccncc5)cc4)ccc32)cc1 ; c1ccc(-c2ccc(-n3c4ccccc4c4cc(-c5cccc6c5c5ccccc5n6-c5ccccc5)ccc43)cc2)cc1] 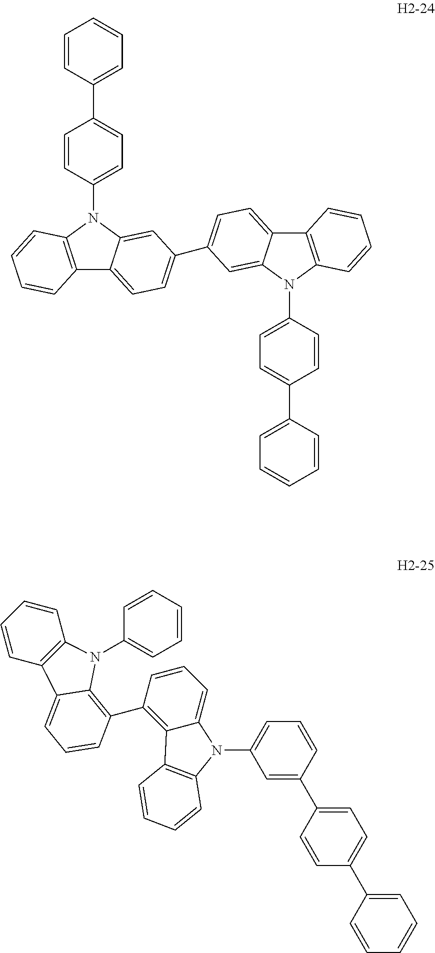 [SMILES: c1ccc(-c2ccc(-n3c4ccccc4c4ccc(-c5ccc6c7ccccc7n(-c7ccc(-c8ccccc8)cc7)c6c5)cc43)cc2)cc1 ; c1ccc(-c2ccc(-c3cccc(-n4c5ccccc5c5c(-c6cccc7c8ccccc8n(-c8ccccc8)c67)cccc54)c3)cc2)cc1] 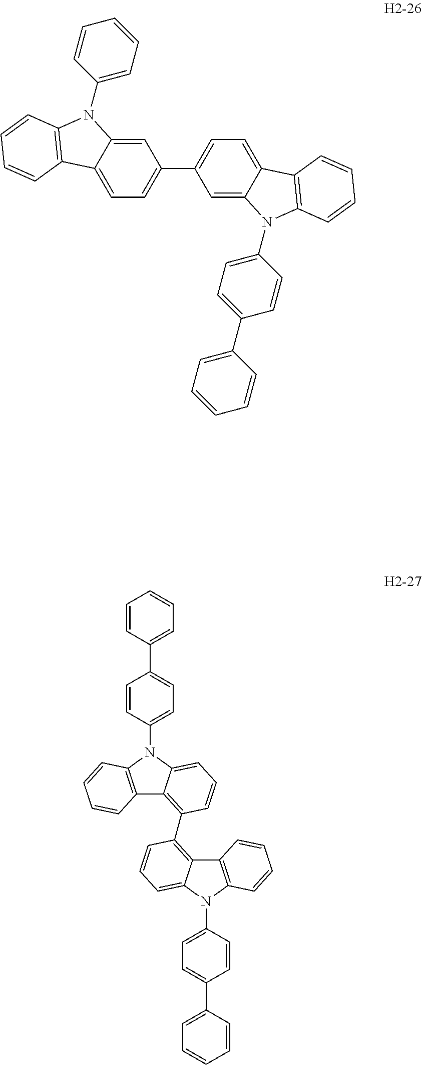 [SMILES: c1ccc(-c2ccc(-n3c4ccccc4c4ccc(-c5ccc6c7ccccc7n(-c7ccccc7)c6c5)cc43)cc2)cc1 ; c1ccc(-c2ccc(-n3c4ccccc4c4c(-c5cccc6c5c5ccccc5n6-c5ccc(-c6ccccc6)cc5)cccc43)cc2)cc1] 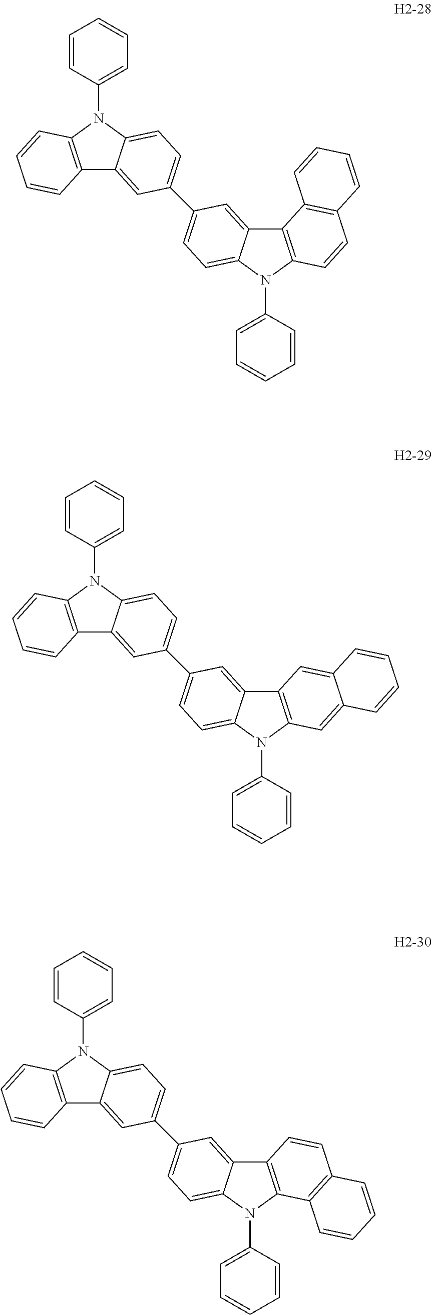 [SMILES: c1ccc(-n2c3ccccc3c3cc(-c4ccc5c(c4)c4c6ccccc6ccc4n5-c4ccccc4)ccc32)cc1 ; c1ccc(-n2c3ccccc3c3cc(-c4ccc5c(c4)c4cc6ccccc6cc4n5-c4ccccc4)ccc32)cc1 ; c1ccc(-n2c3ccccc3c3cc(-c4ccc5c(c4)c4ccc6ccccc6c4n5-c4ccccc4)ccc32)cc1] 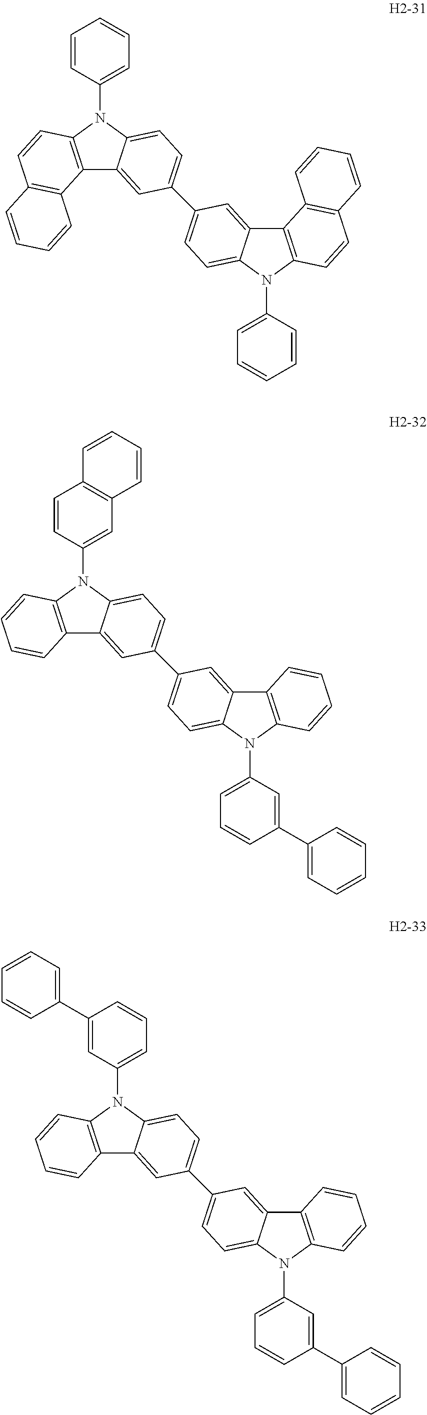 [SMILES: c1ccc(-n2c3ccc(-c4ccc5c(c4)c4c6ccccc6ccc4n5-c4ccccc4)cc3c3c4ccccc4ccc32)cc1 ; c1ccc(-c2cccc(-n3c4ccccc4c4cc(-c5ccc6c(c5)c5ccccc5n6-c5ccc6ccccc6c5)ccc43)c2)cc1 ; c1ccc(-c2cccc(-n3c4ccccc4c4cc(-c5ccc6c(c5)c5ccccc5n6-c5cccc(-c6ccccc6)c5)ccc43)c2)cc1] 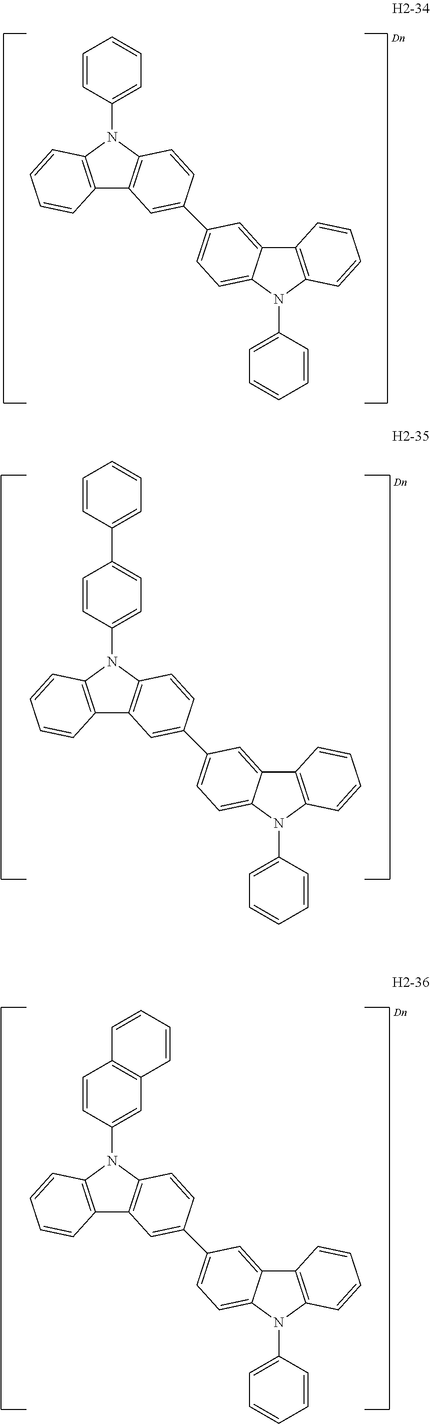 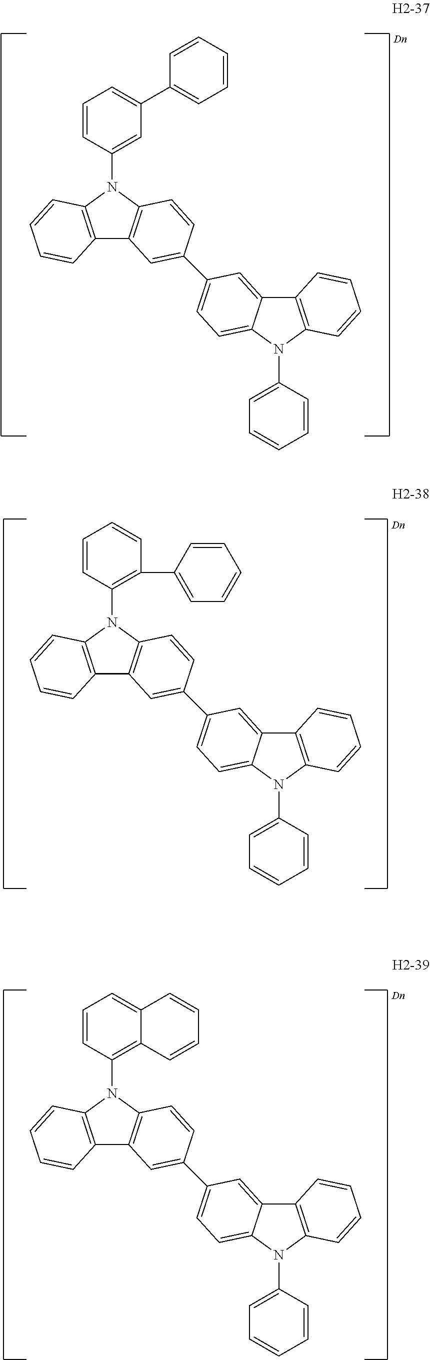 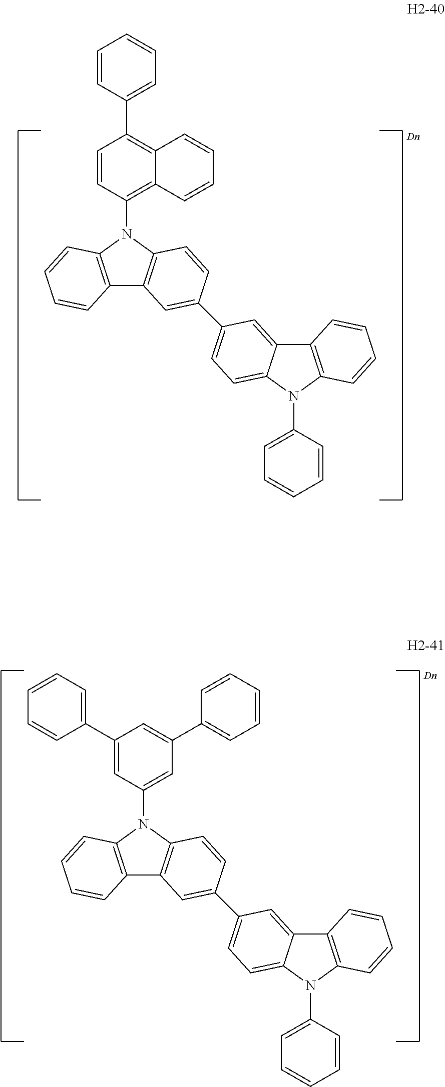 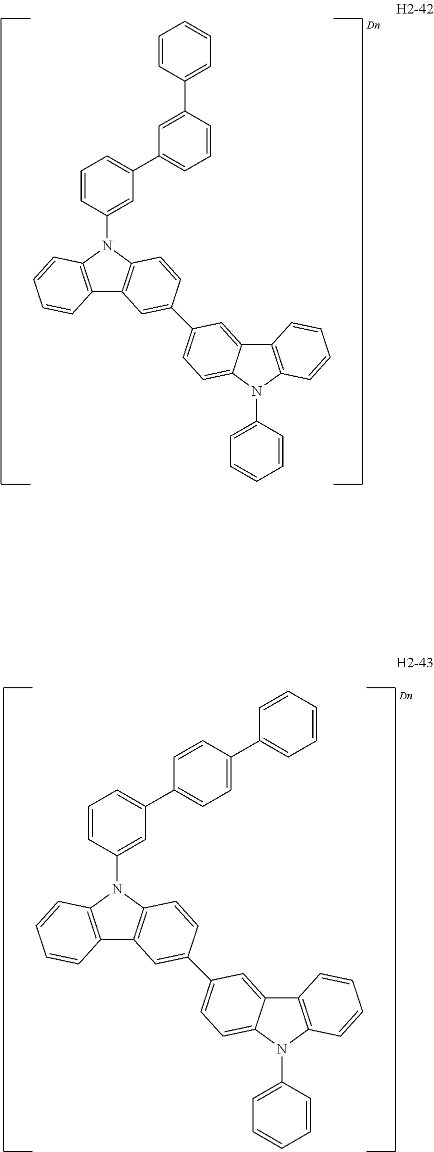 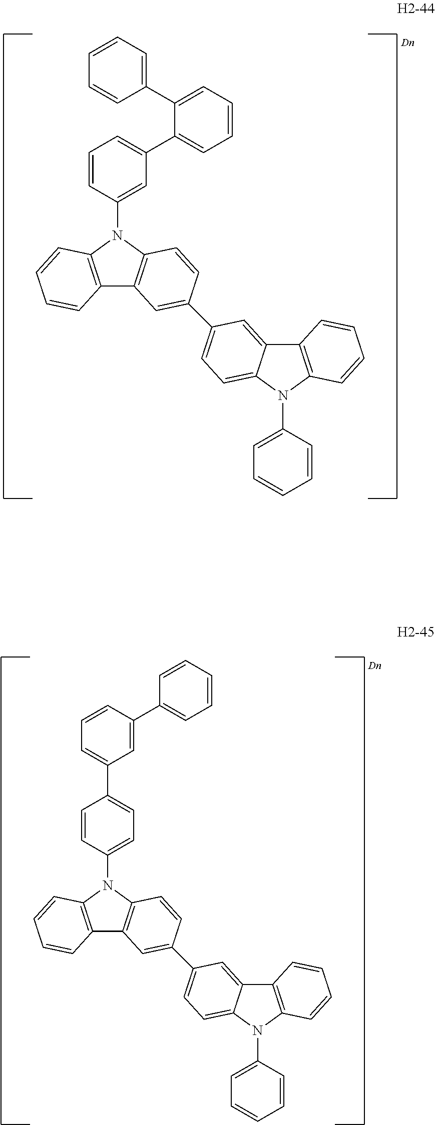 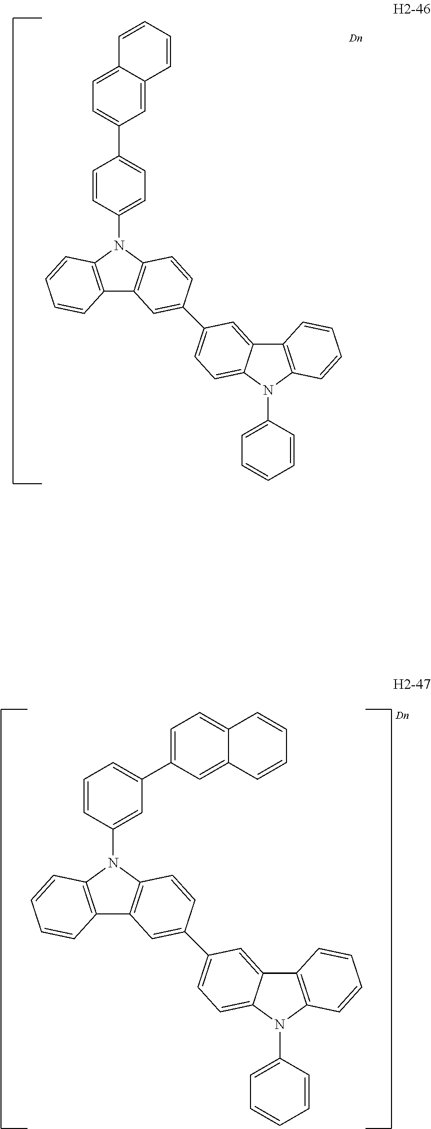 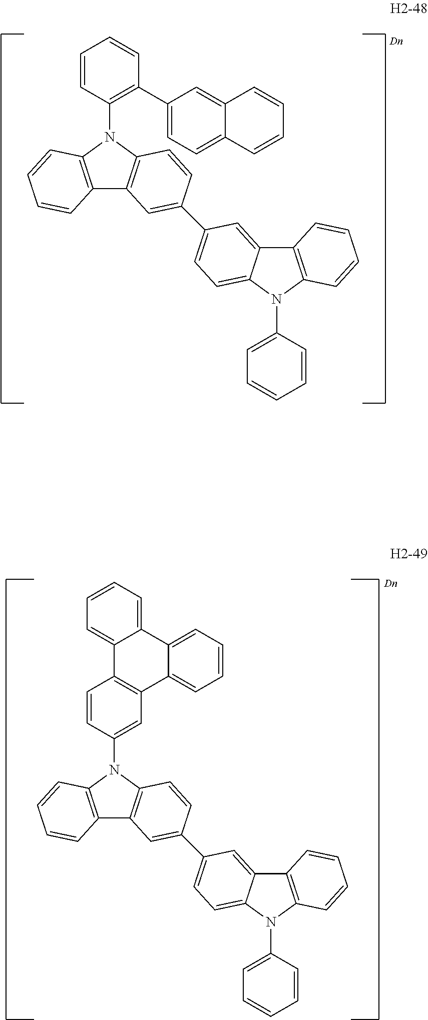 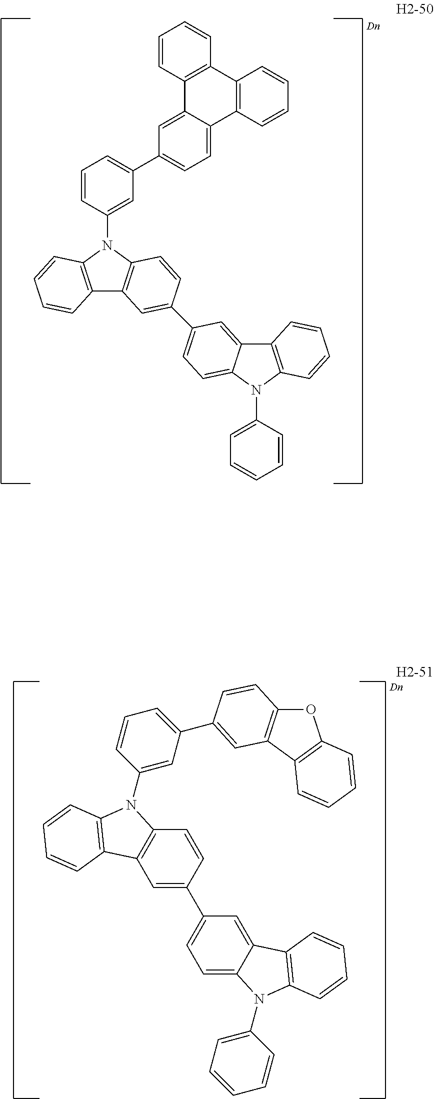 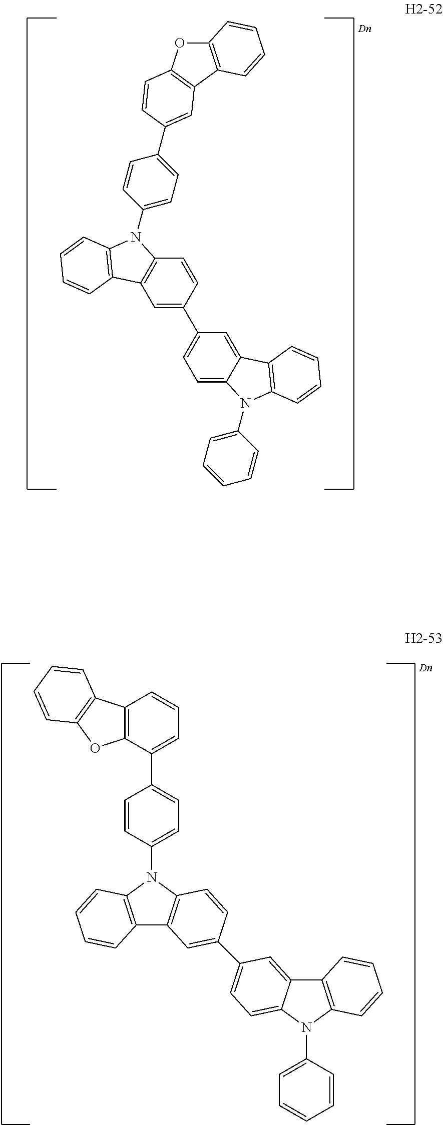 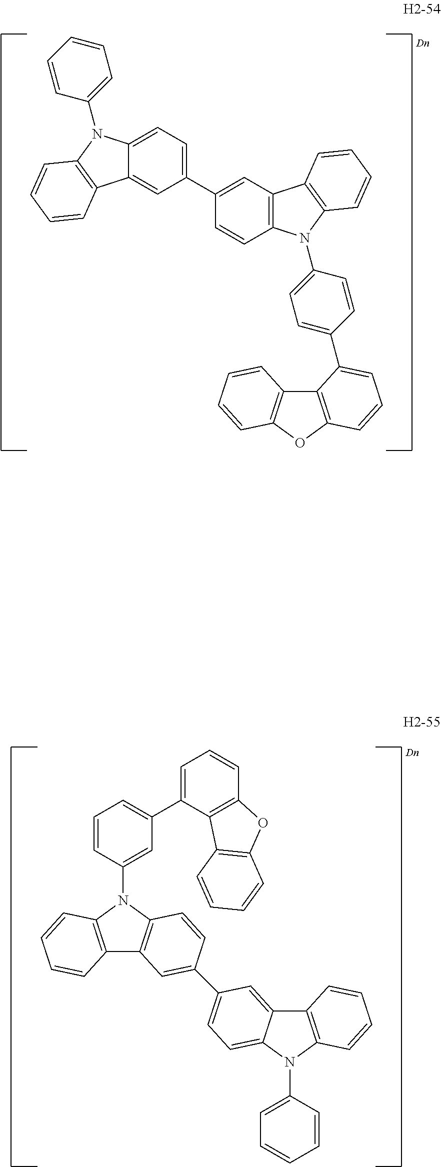 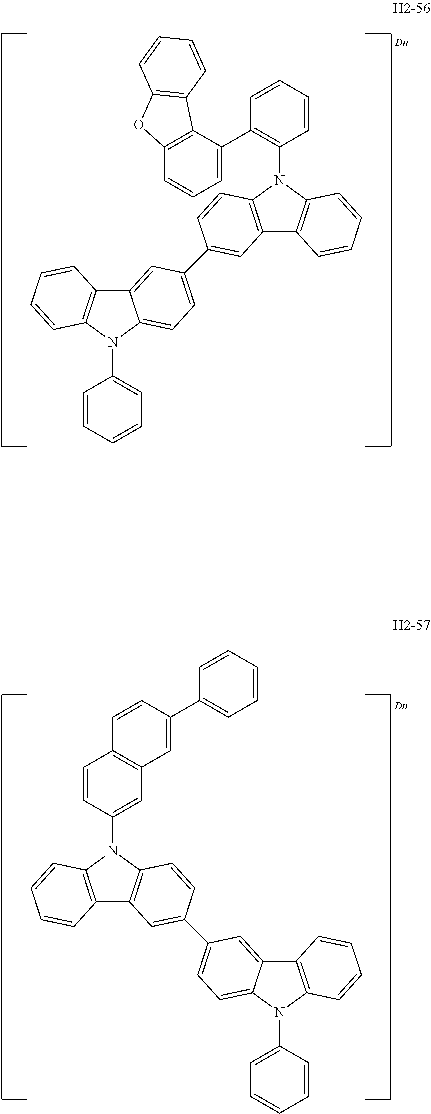 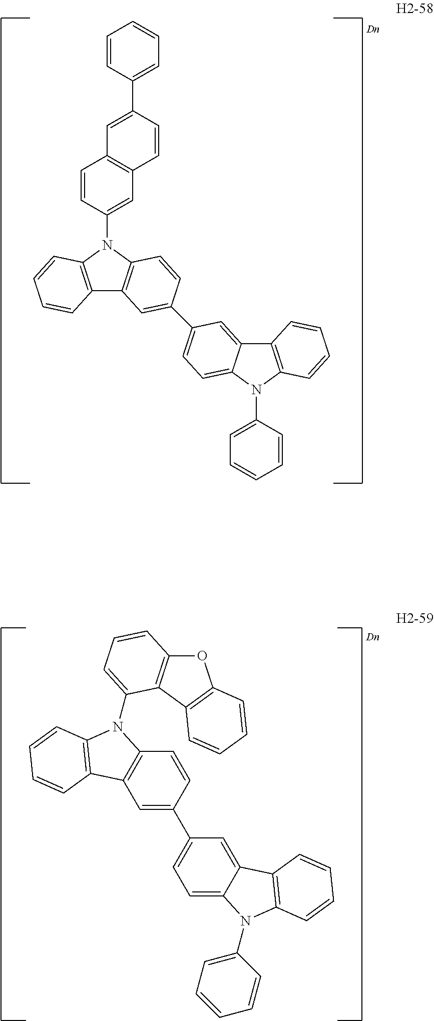 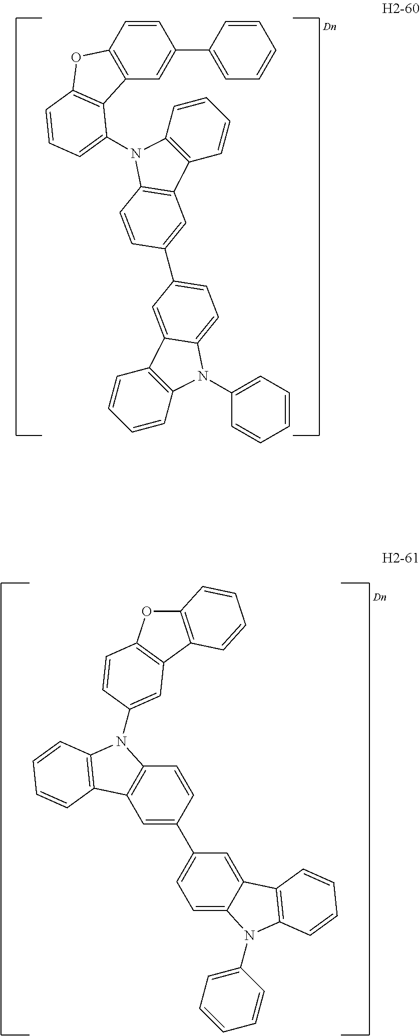 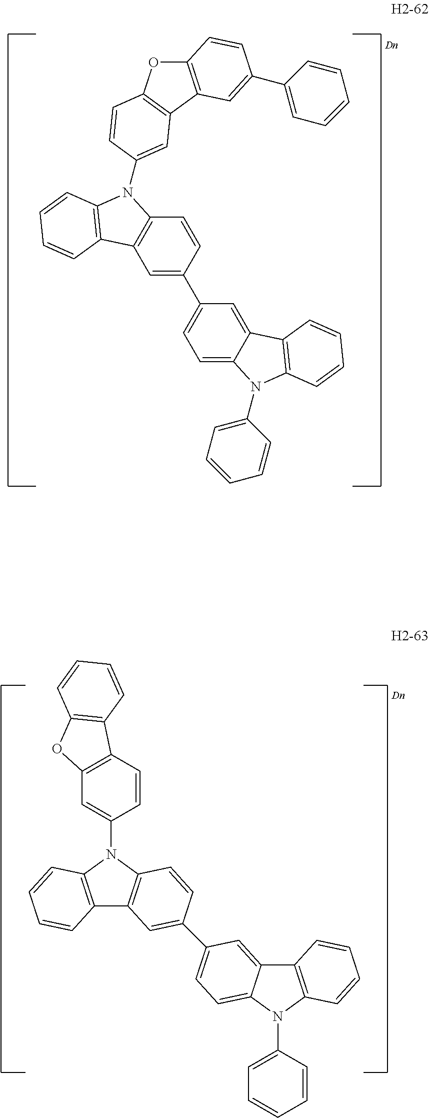 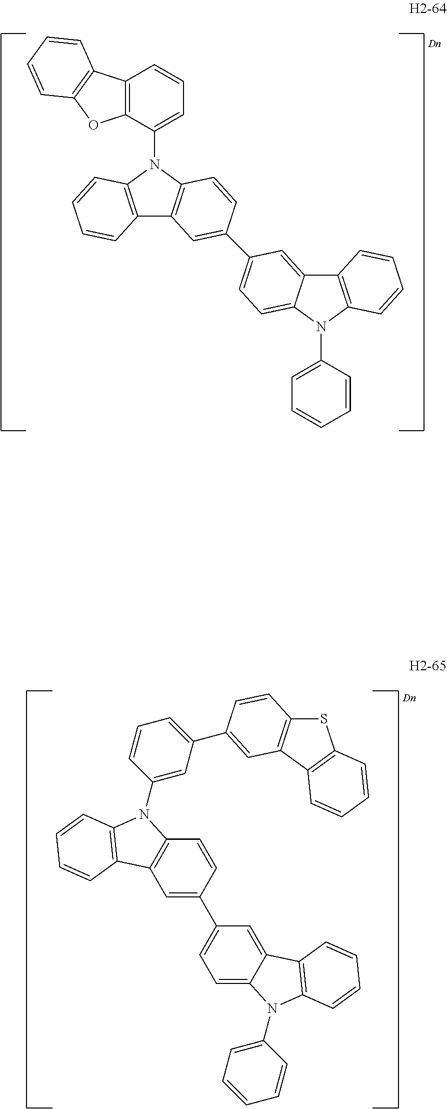 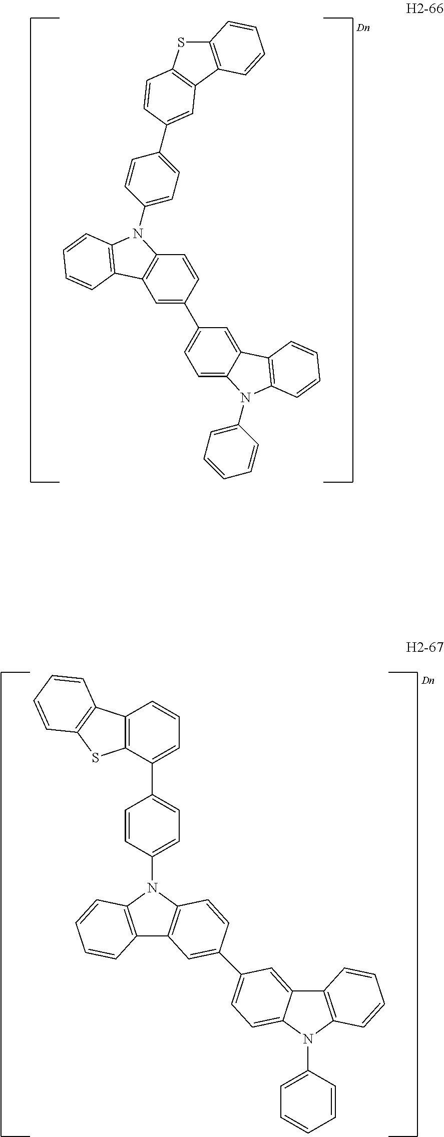 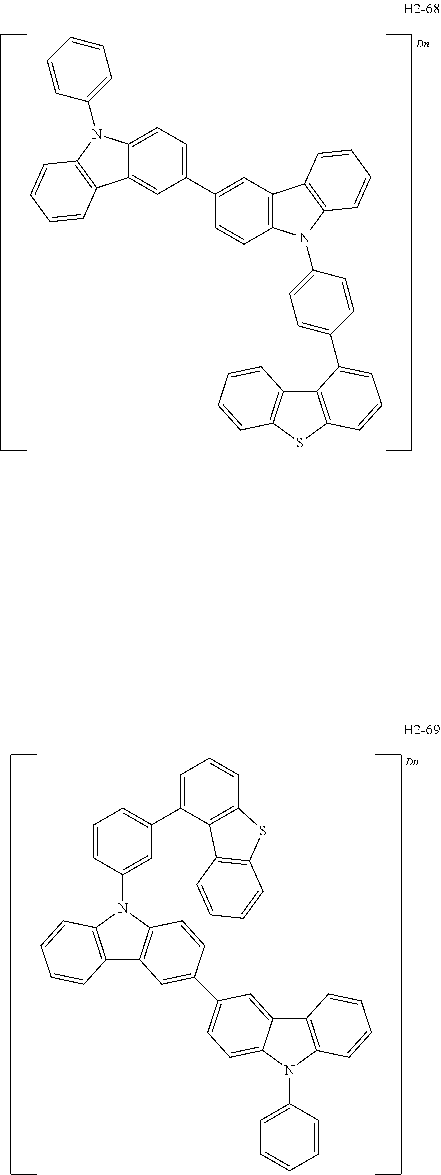 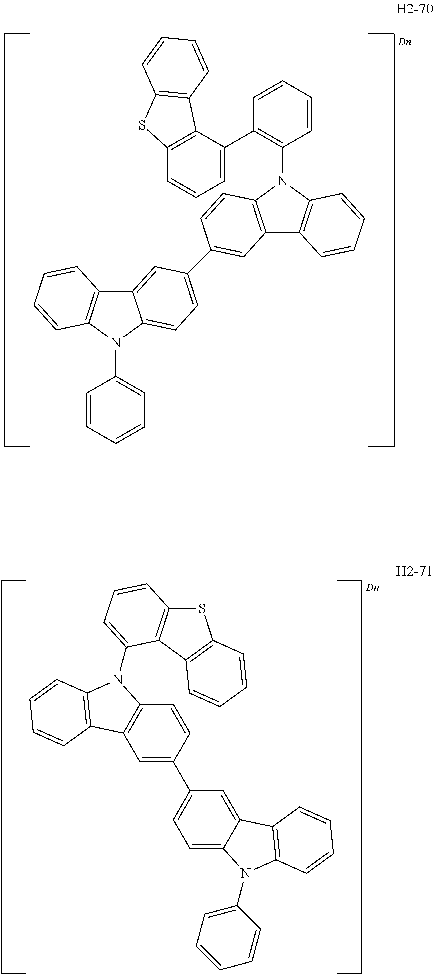 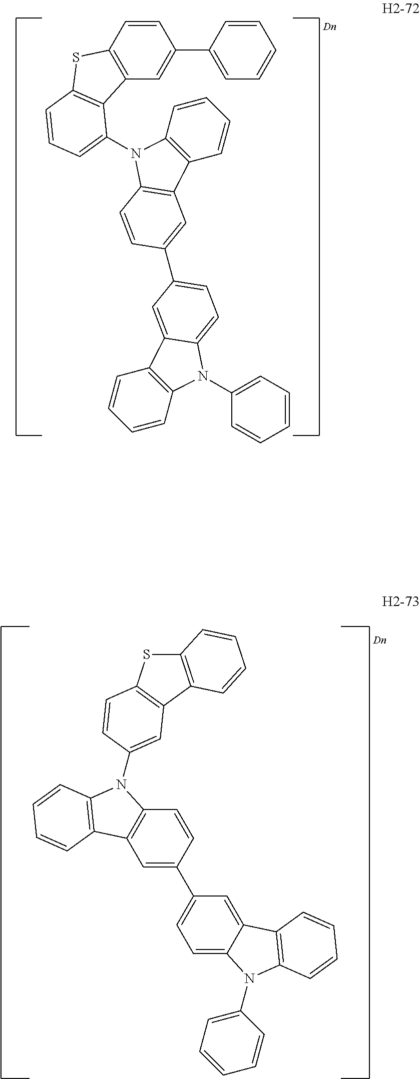 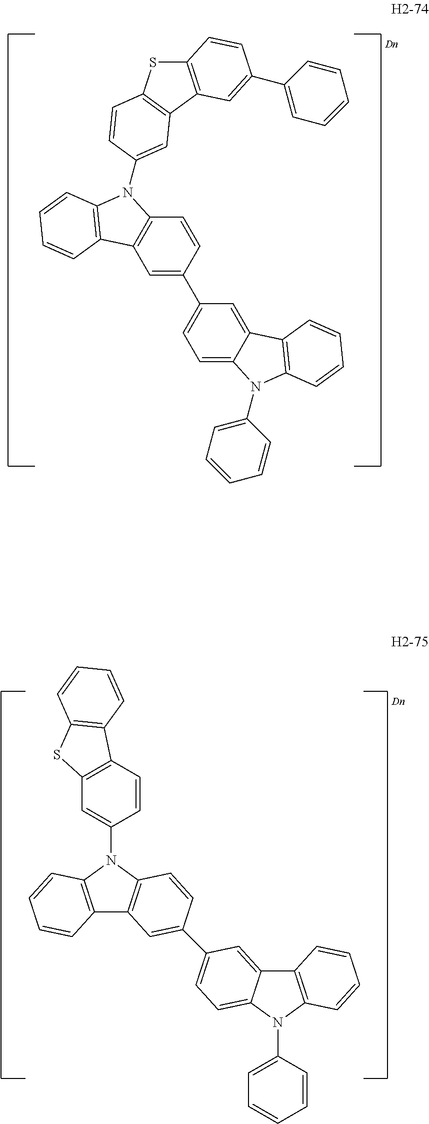 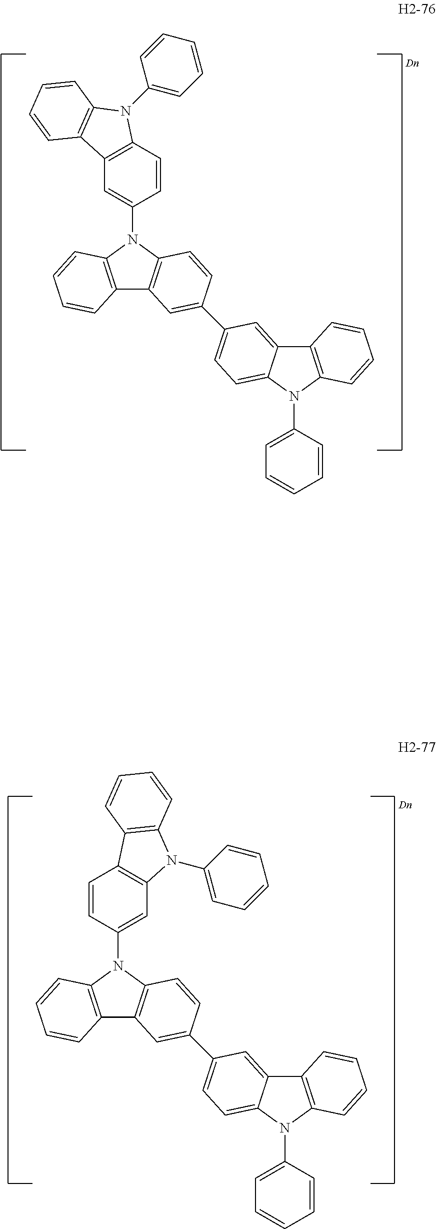 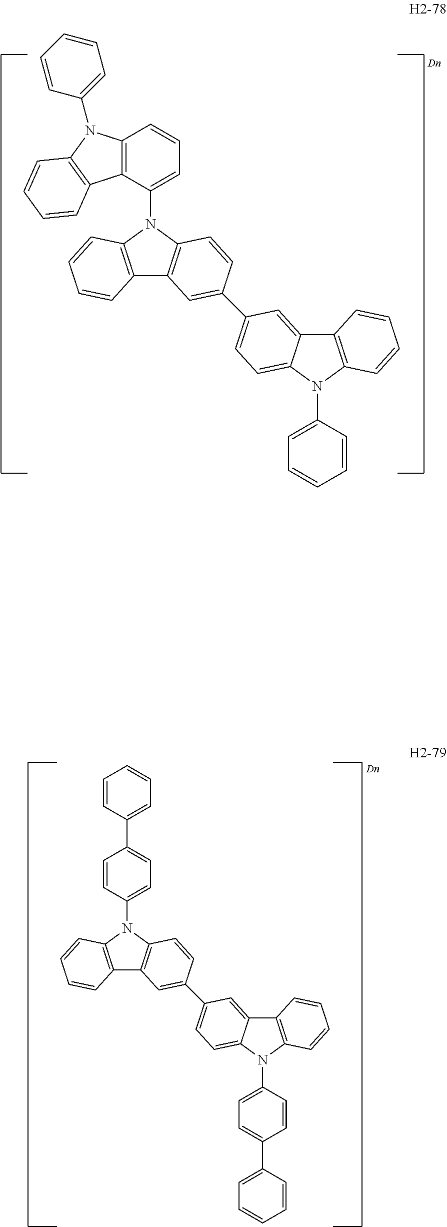 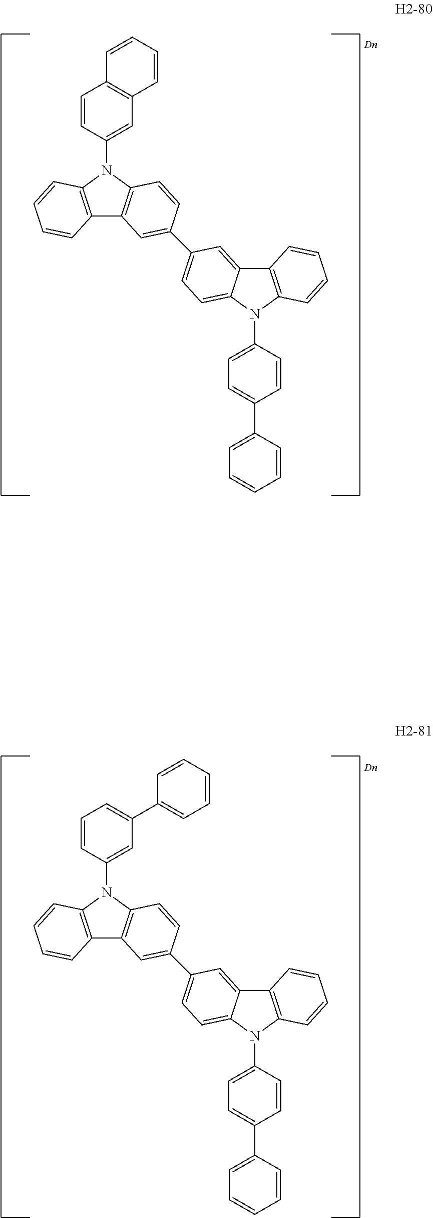 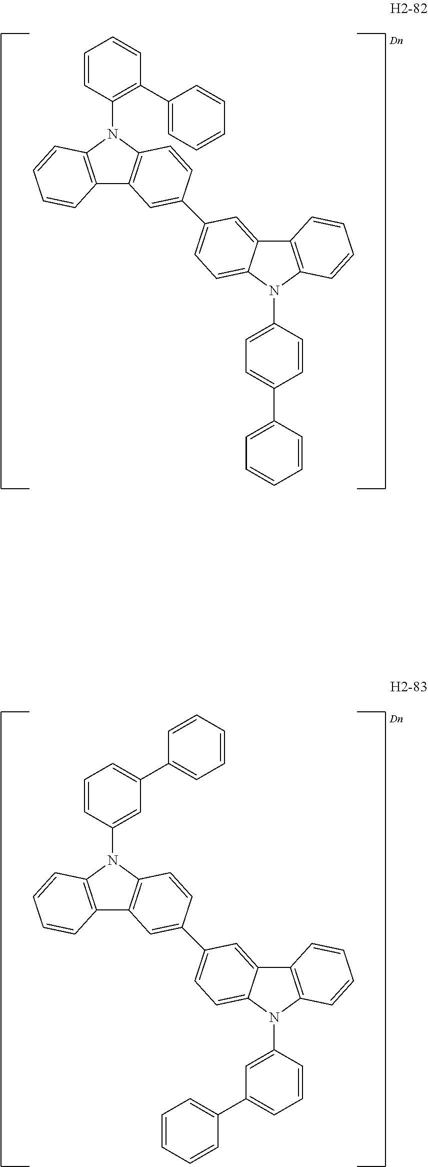 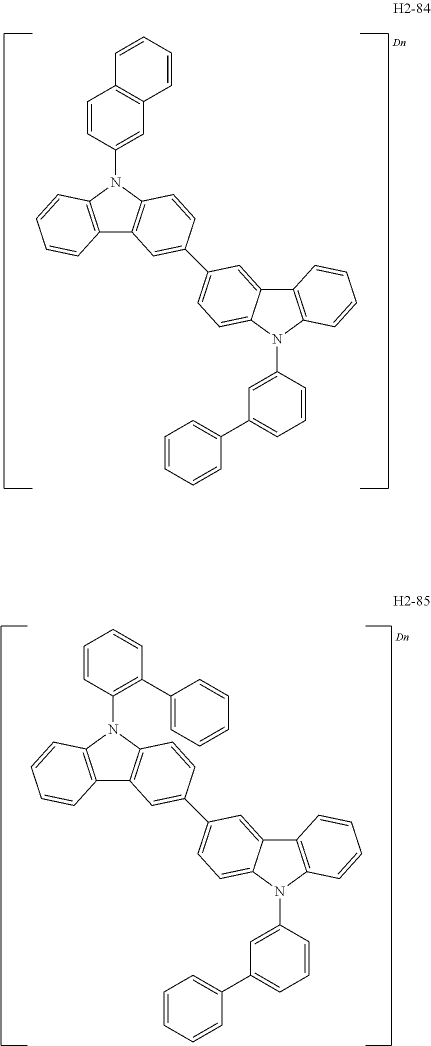 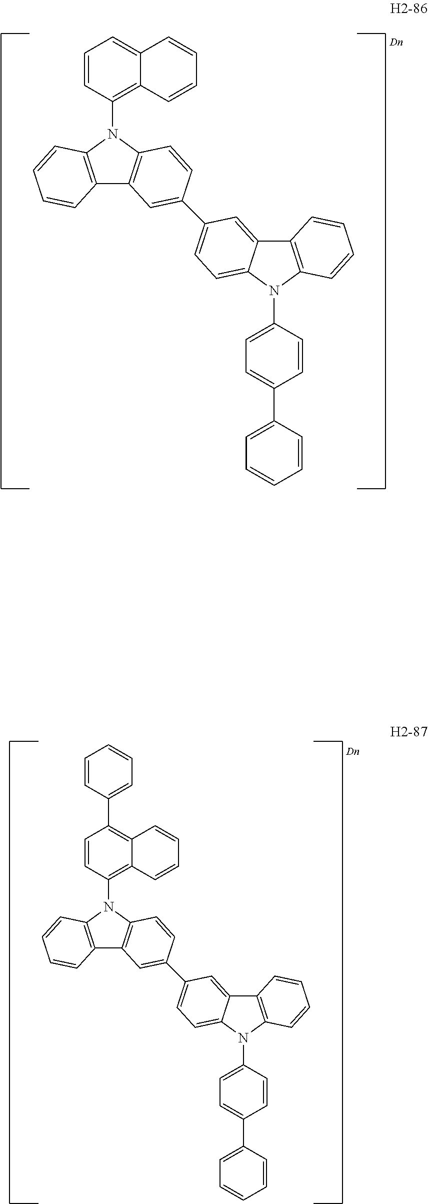 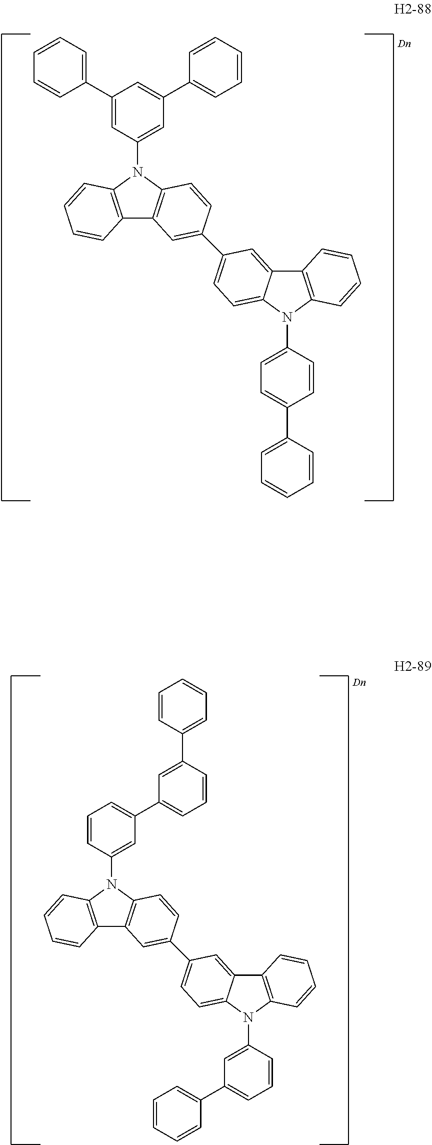 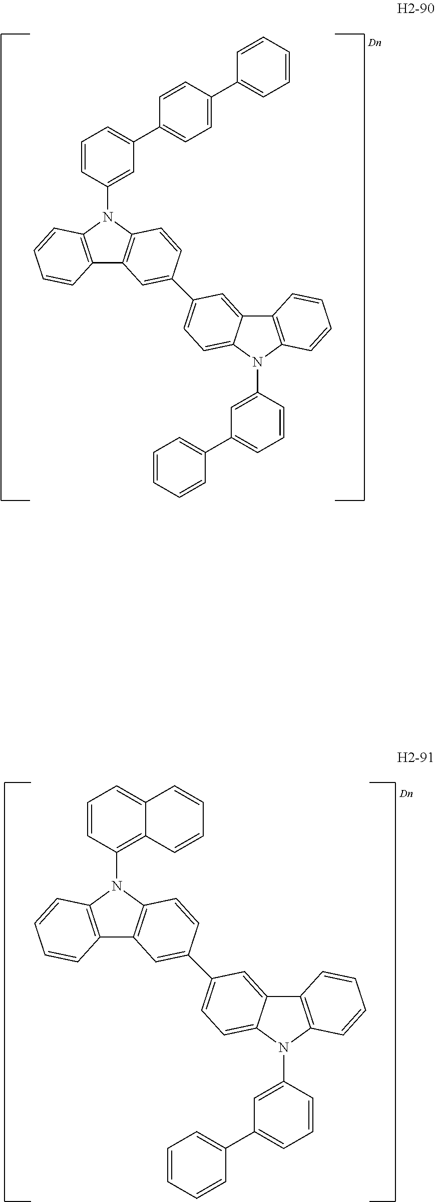 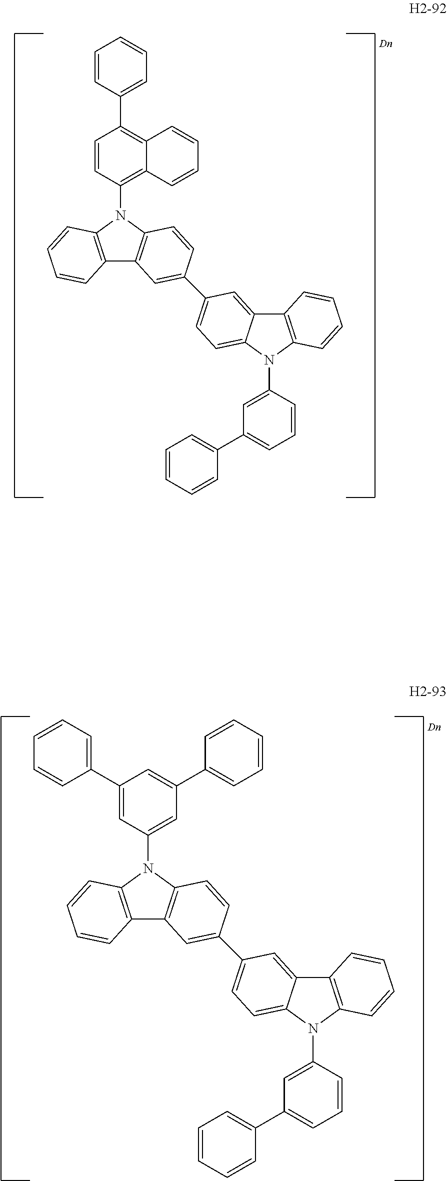 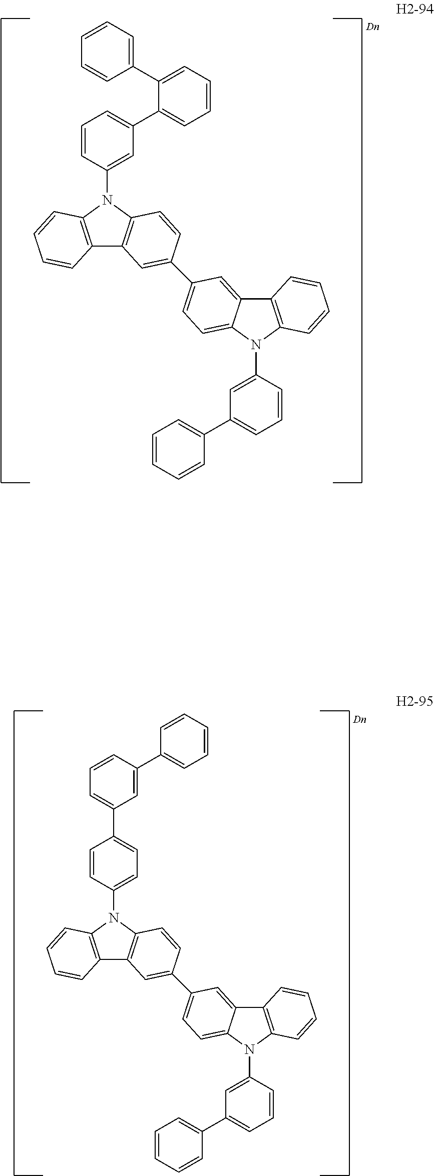 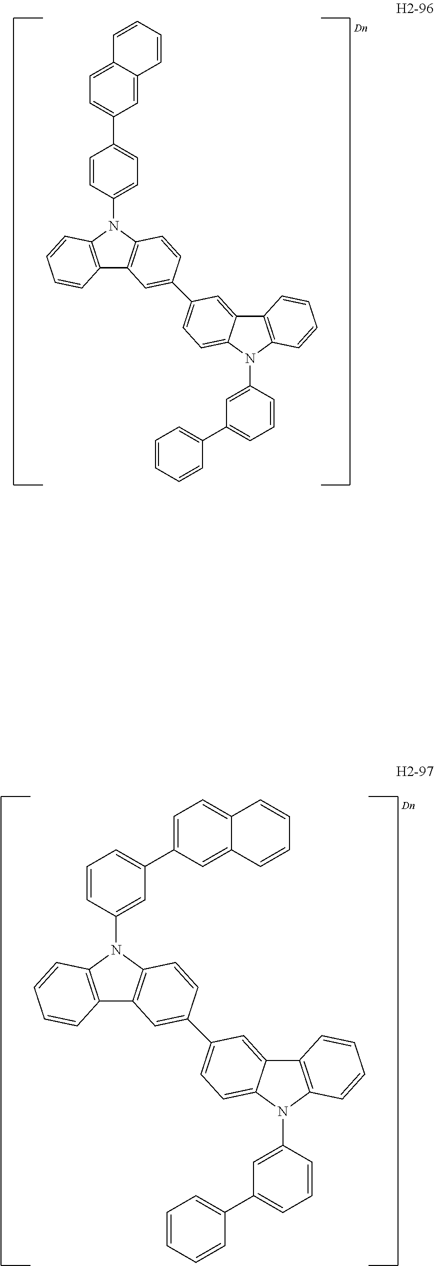 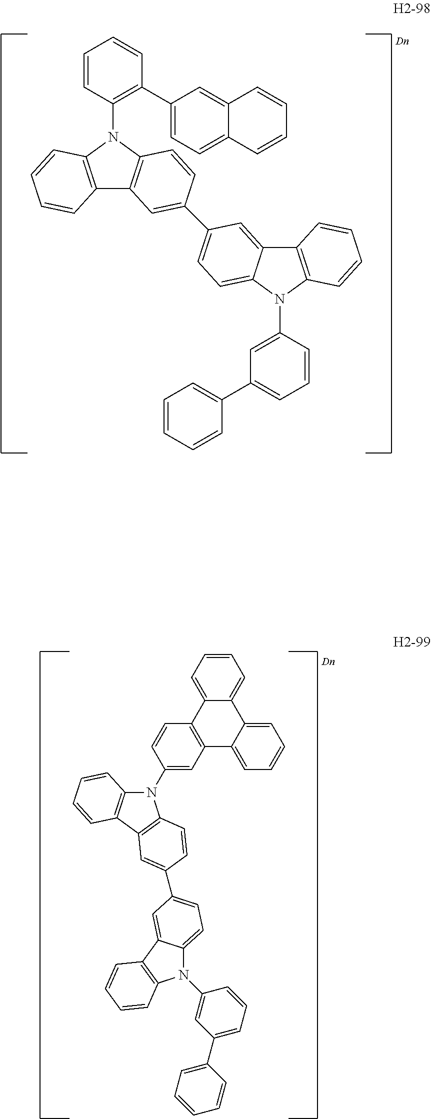 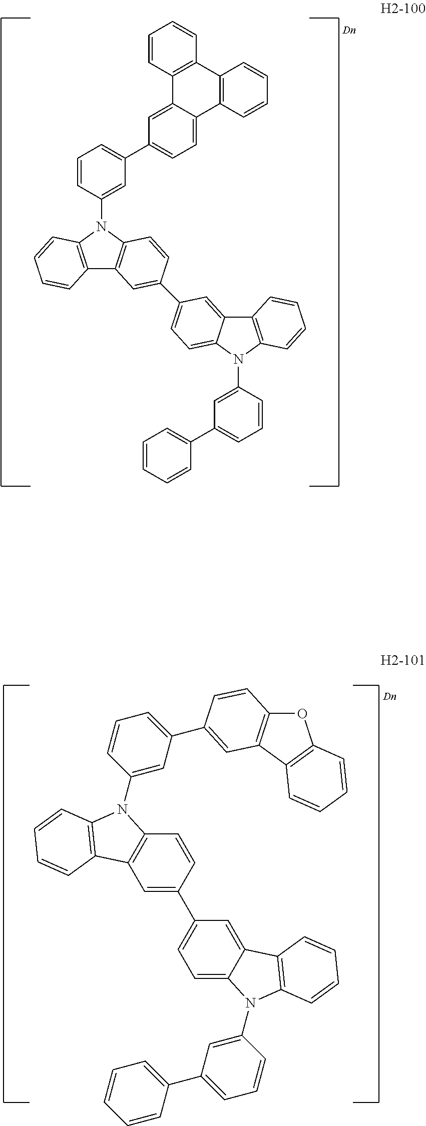 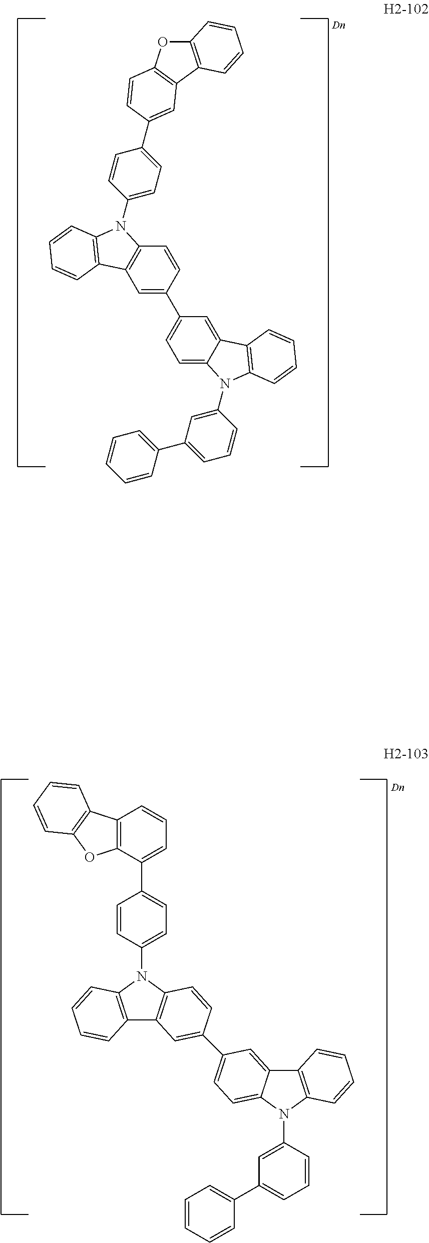 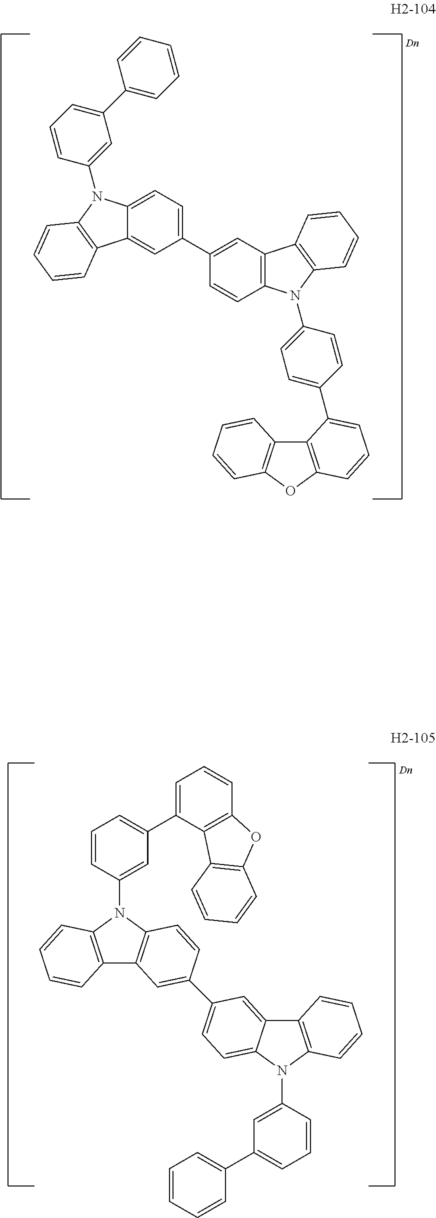 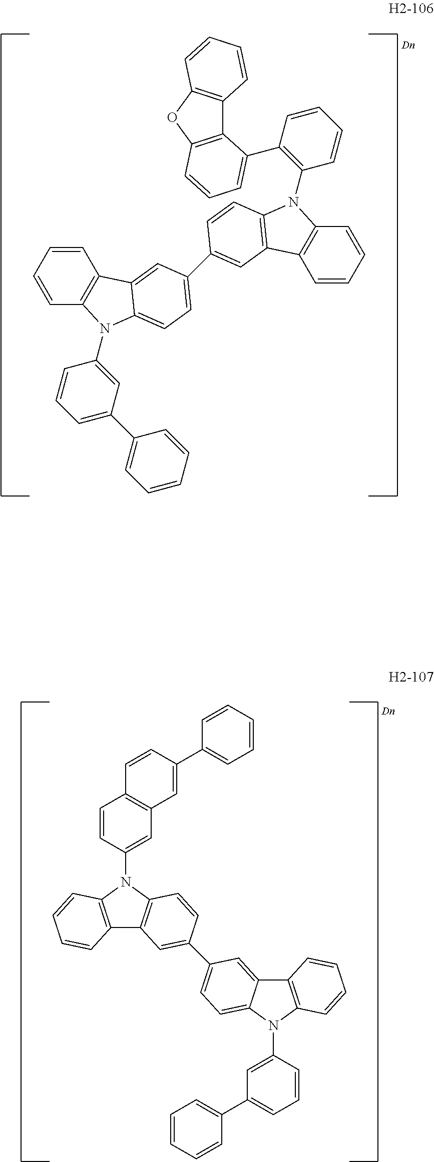 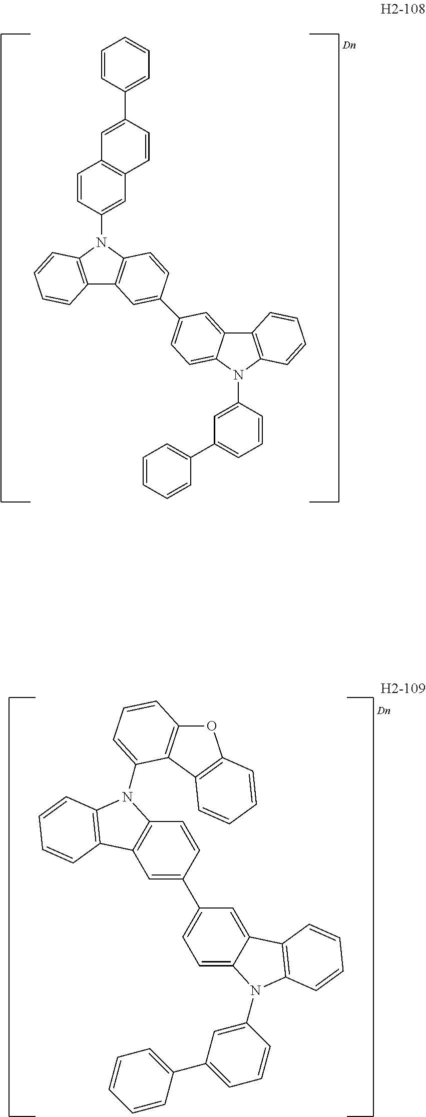 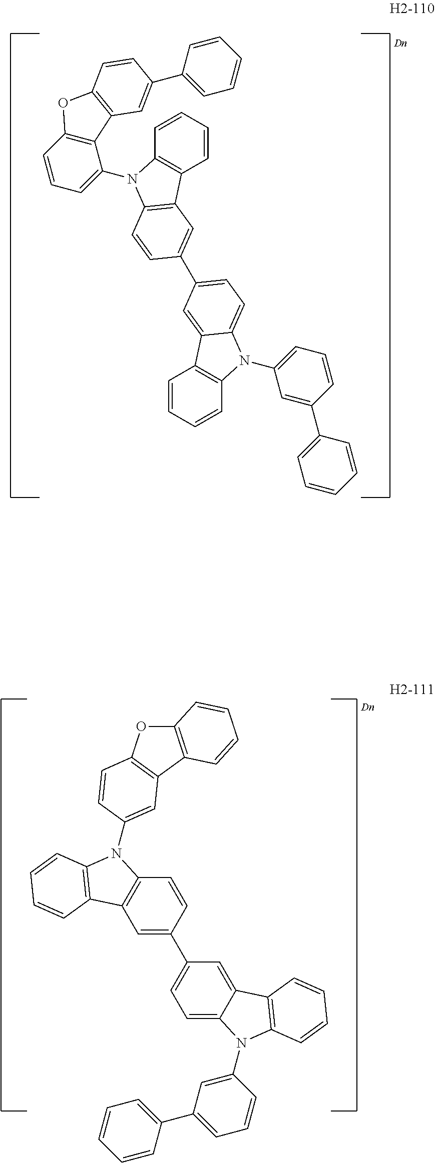 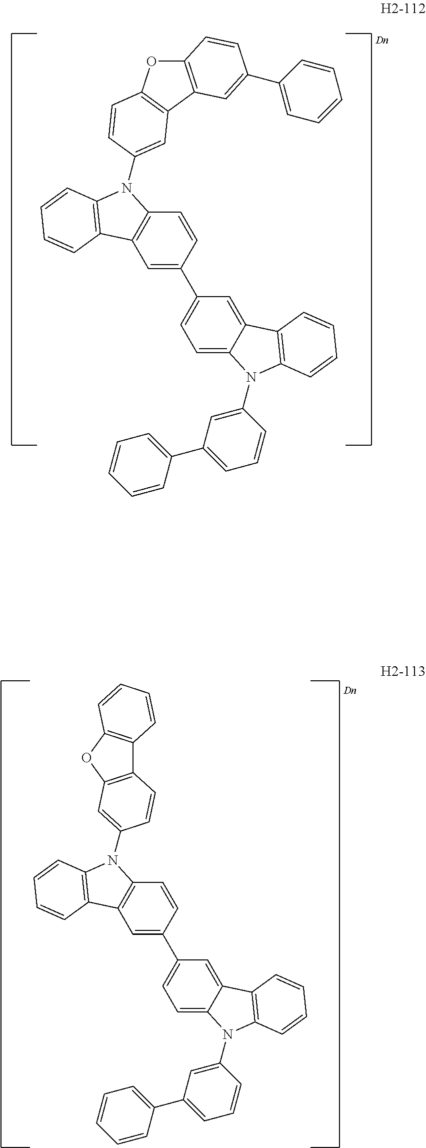 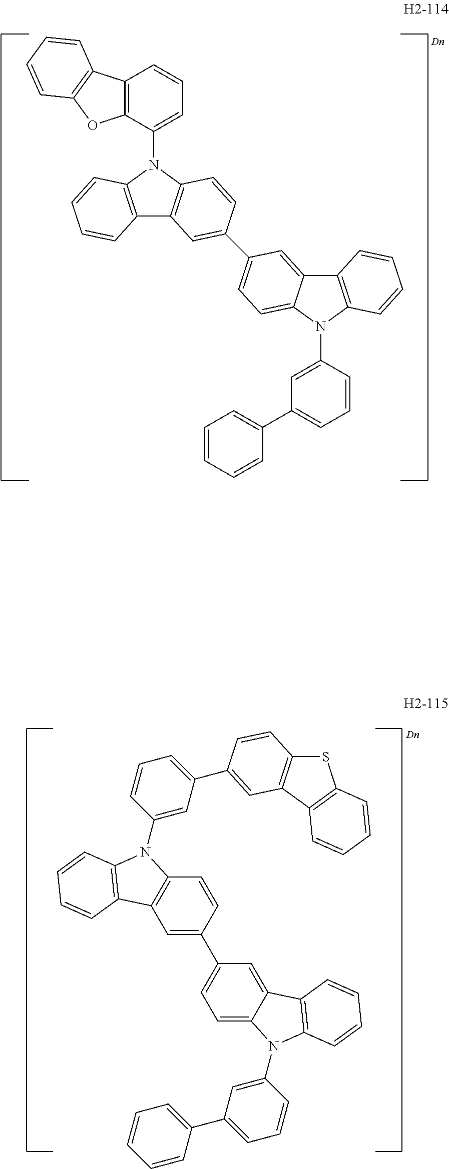 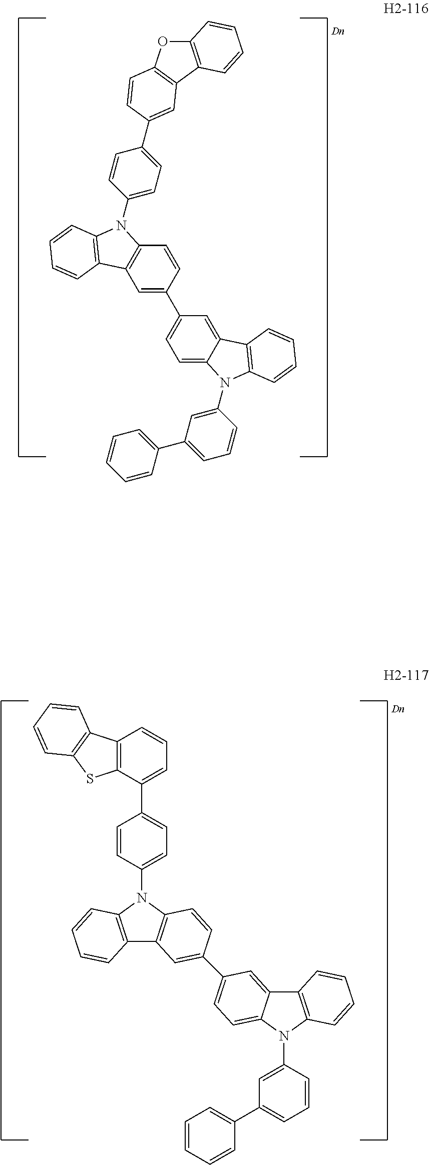 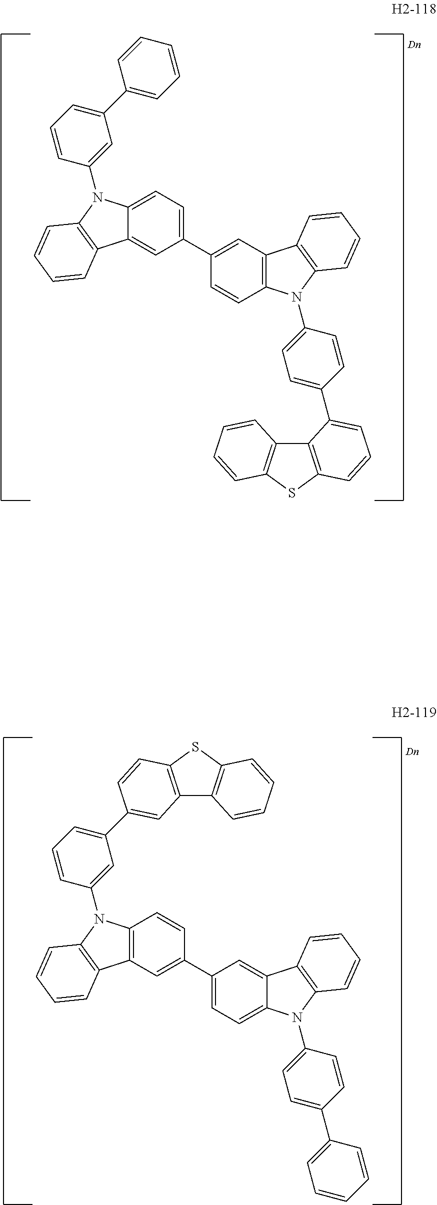 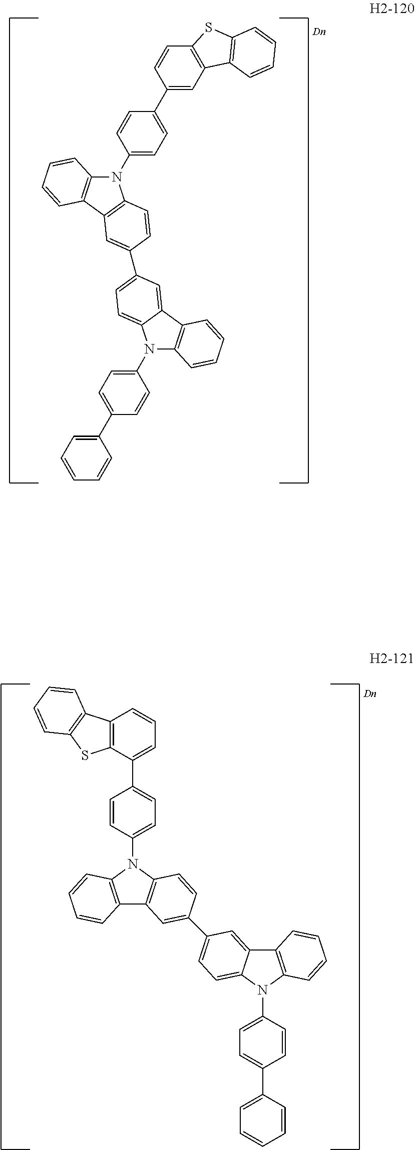 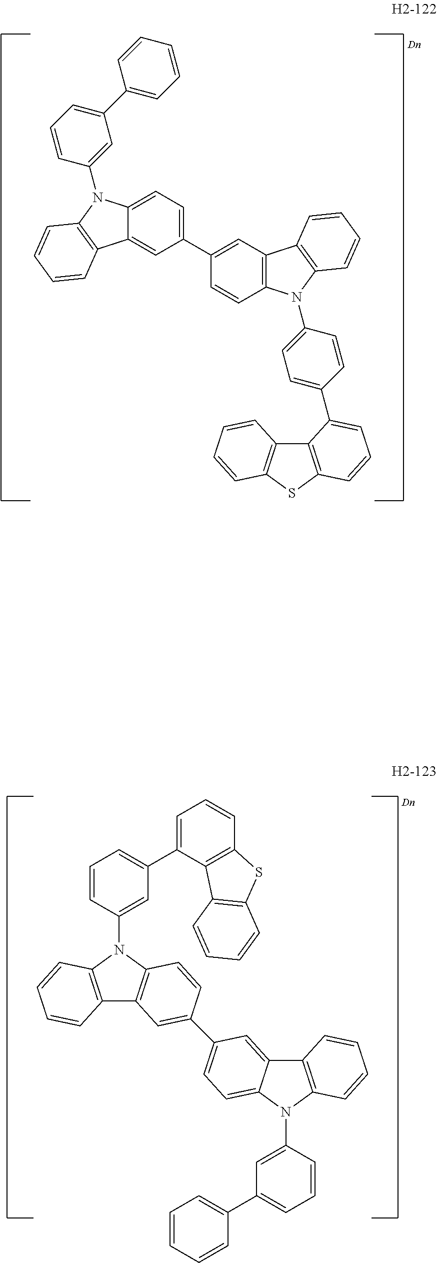 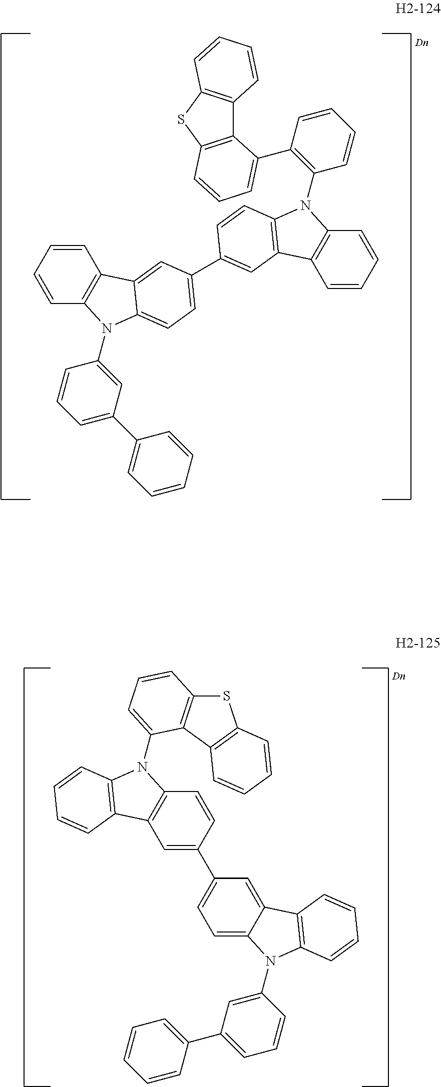 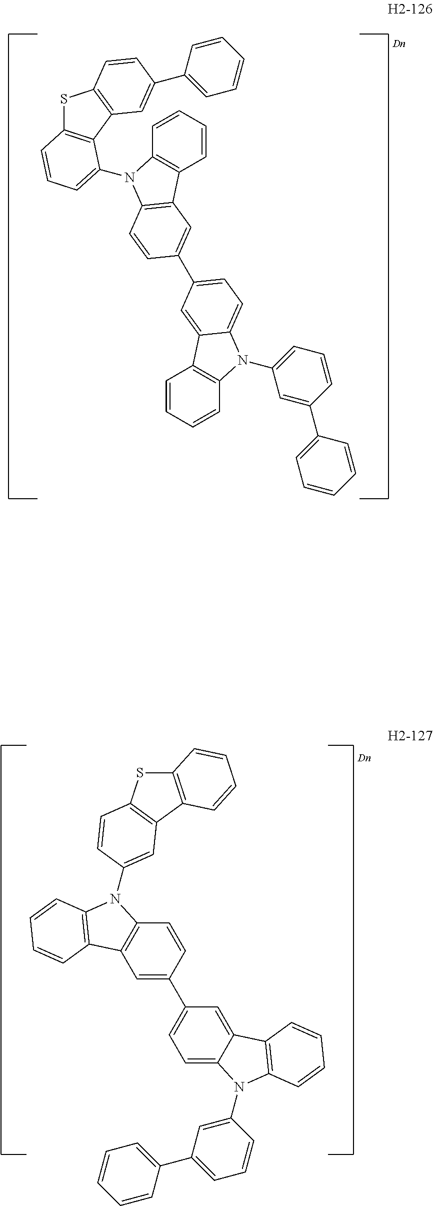 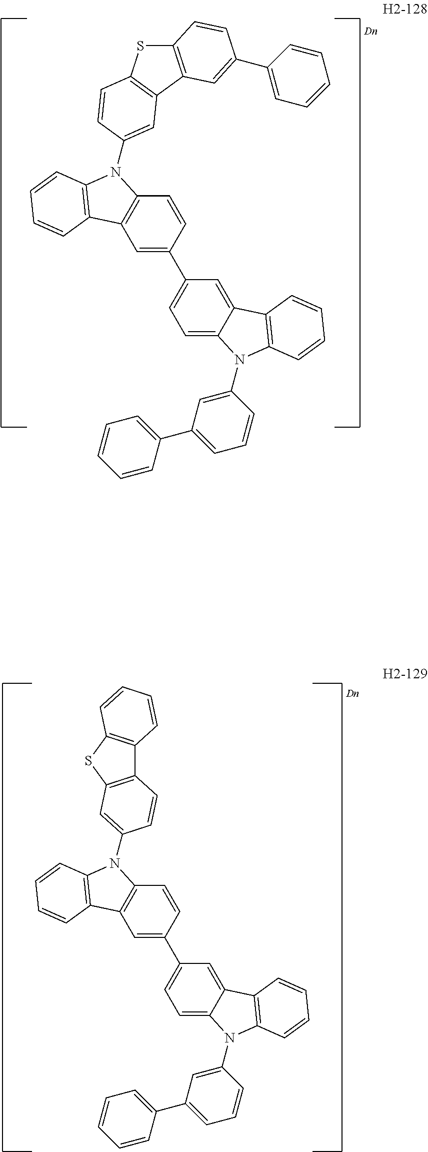 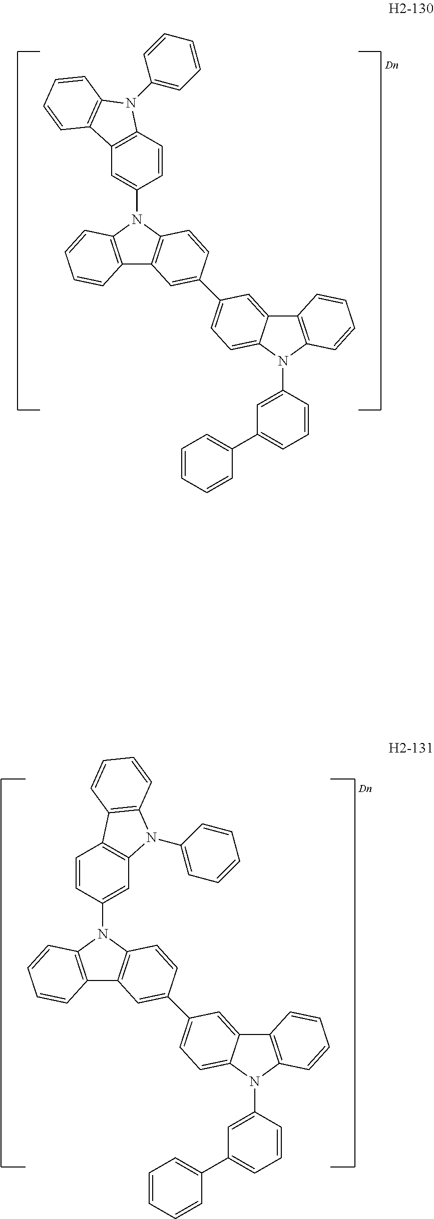 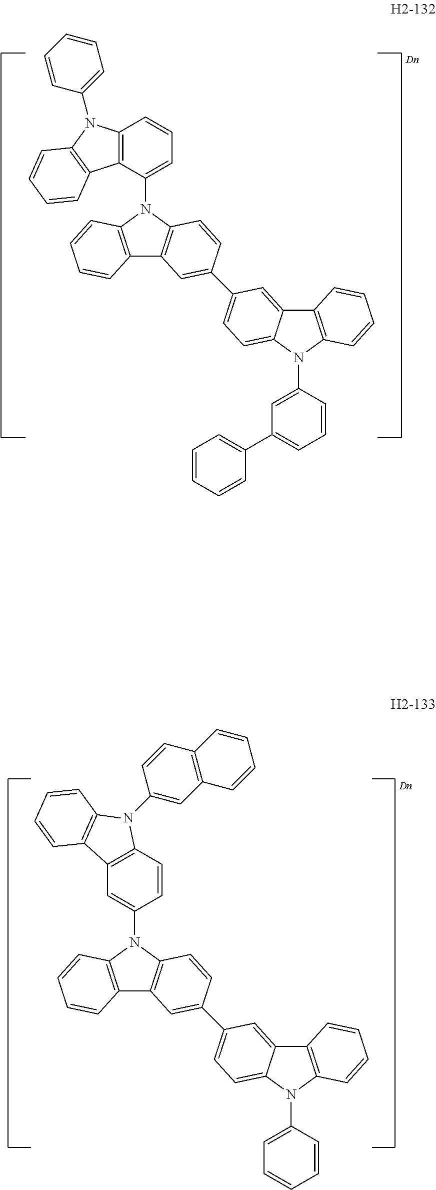 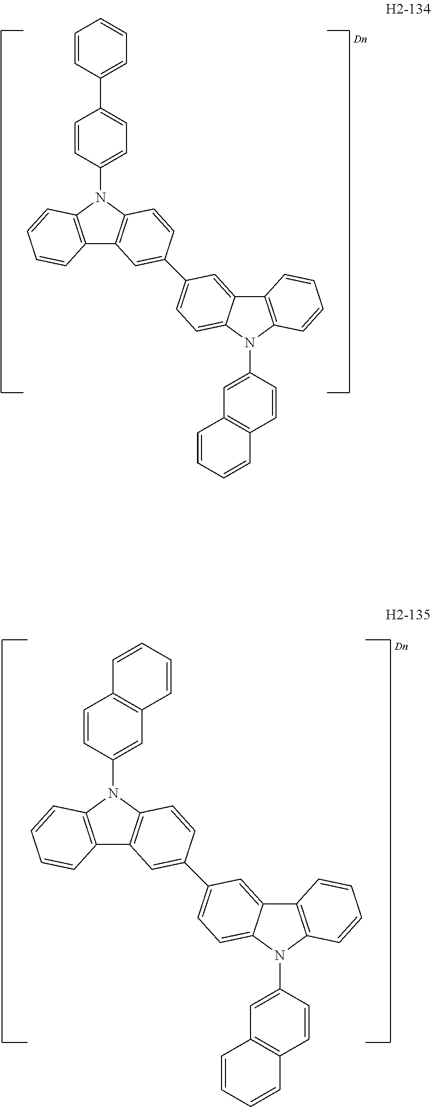 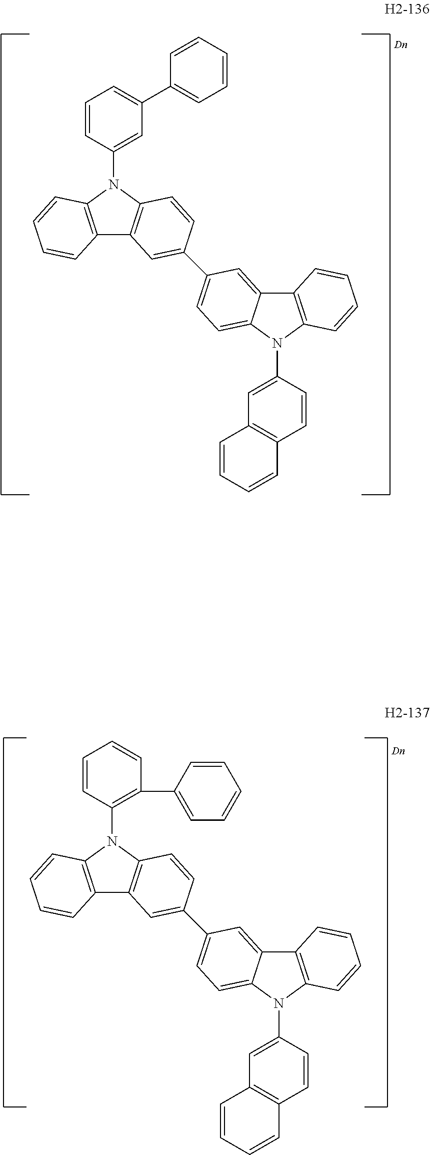 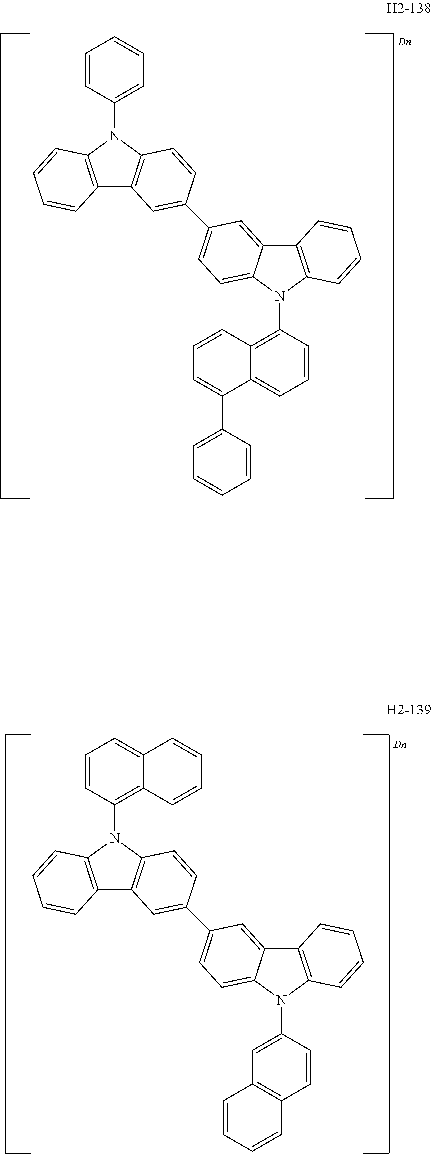 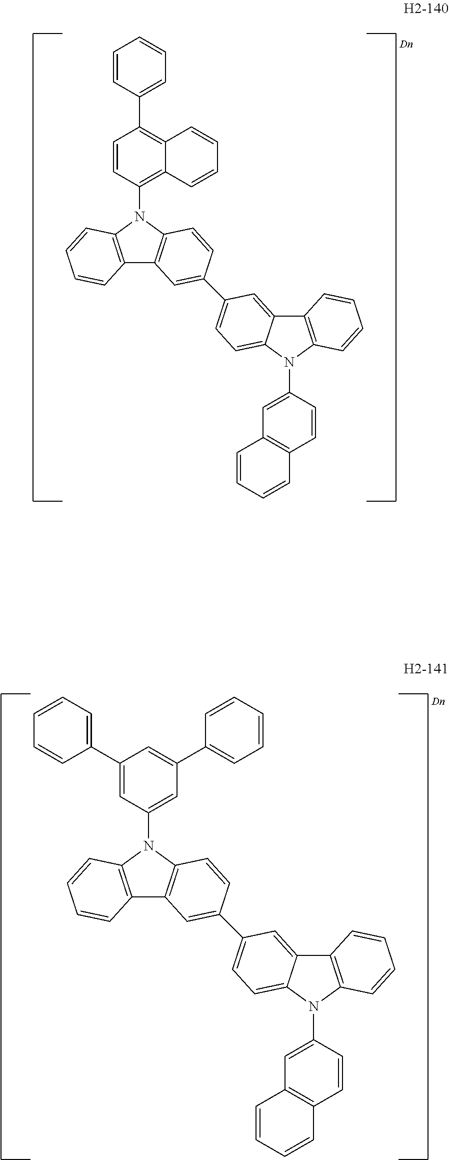 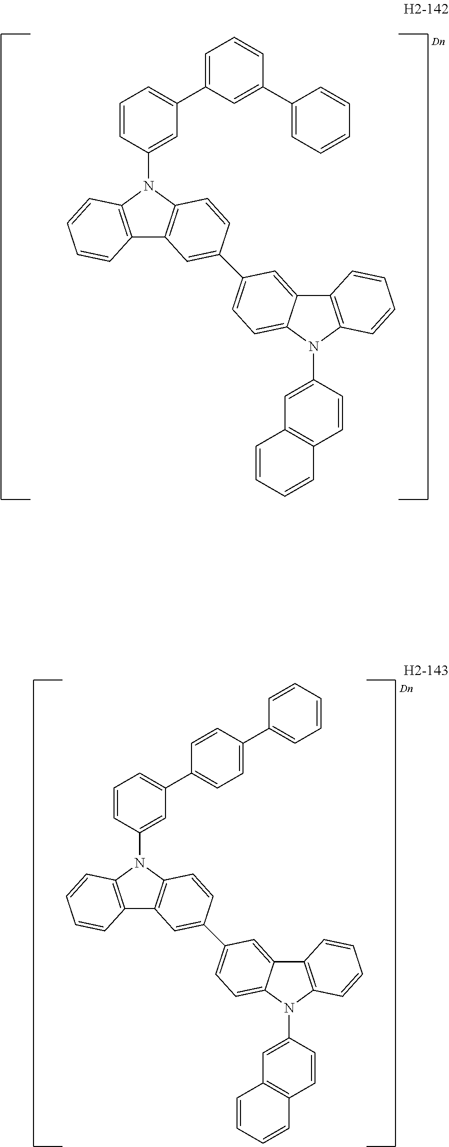 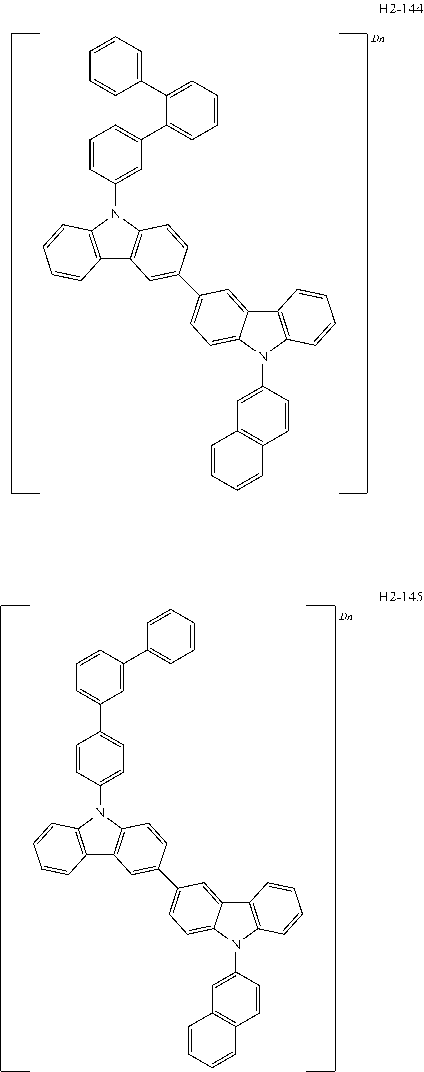 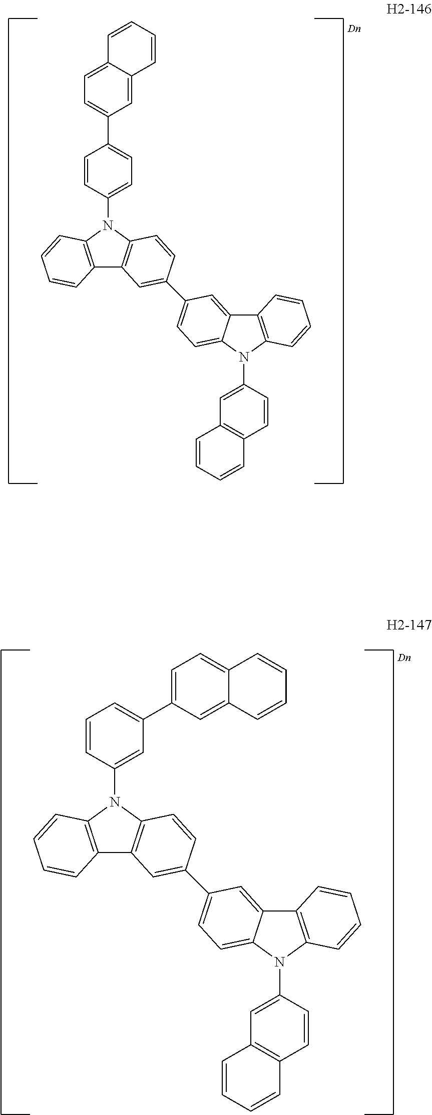 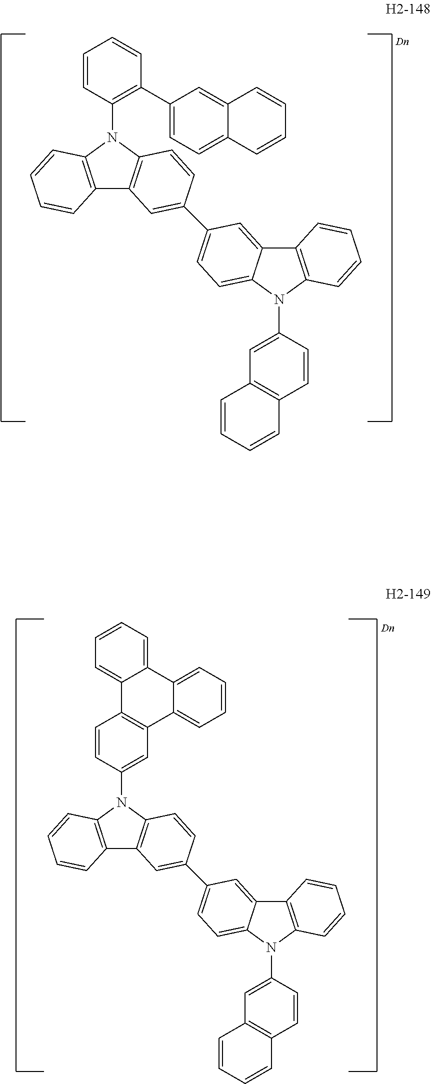 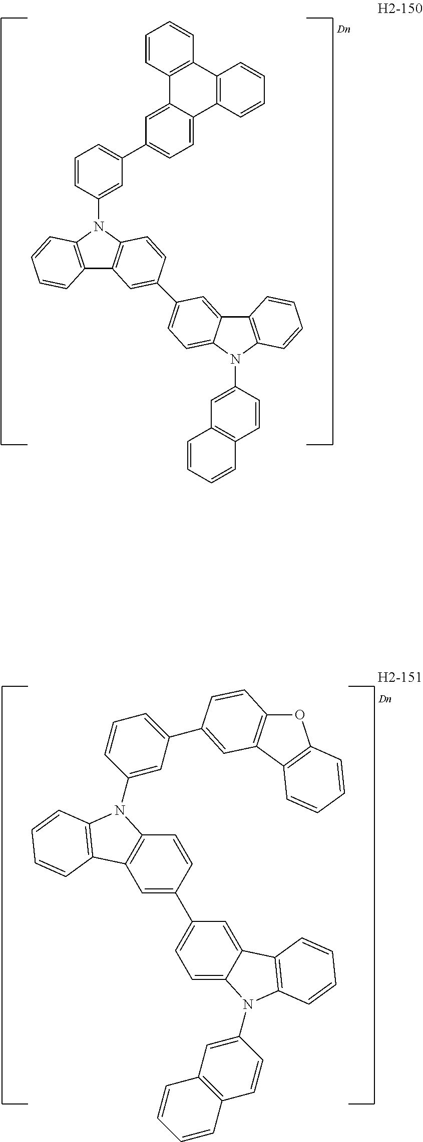 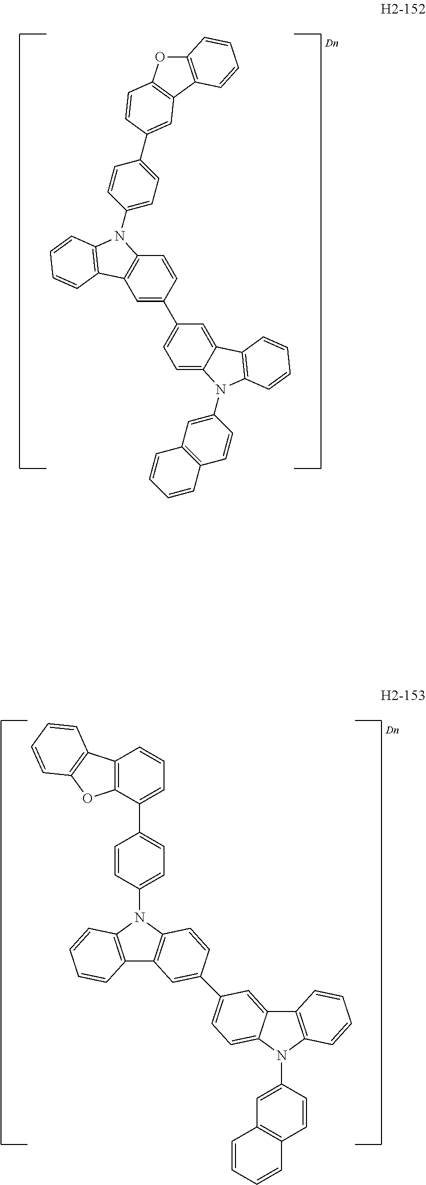 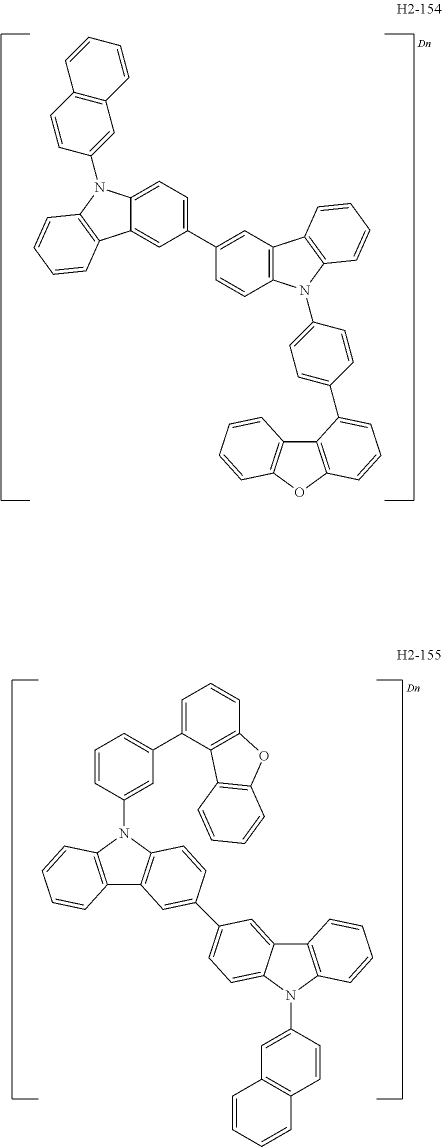 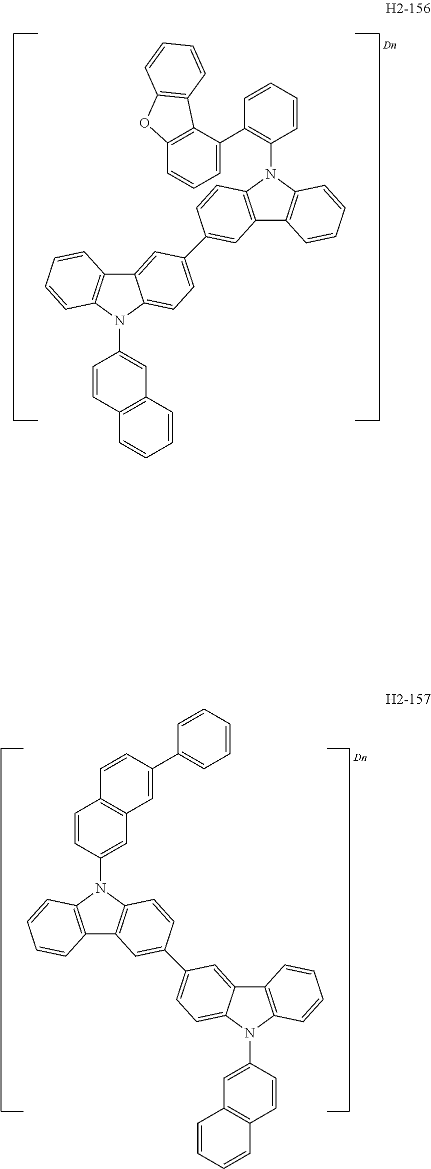 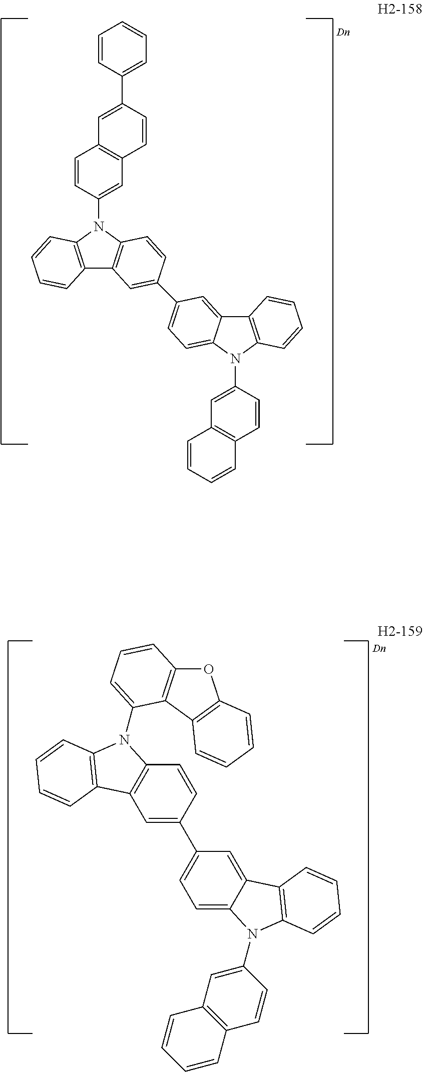 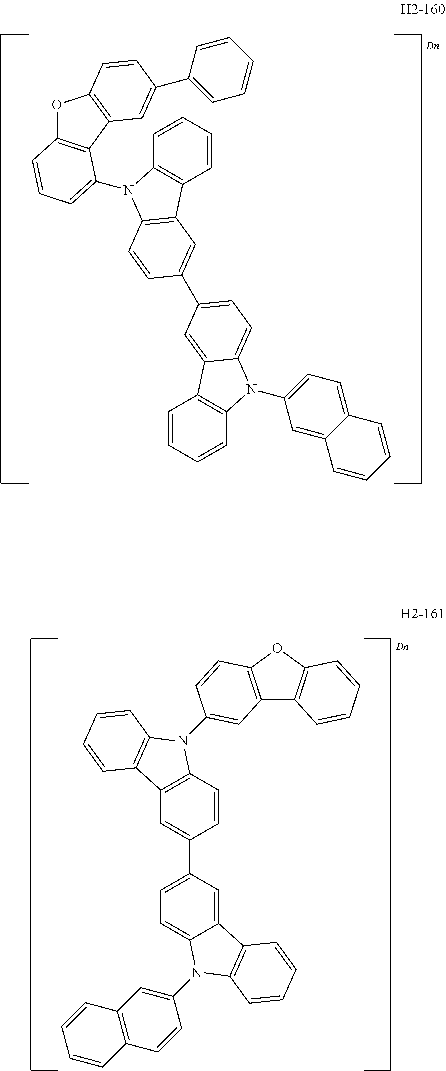 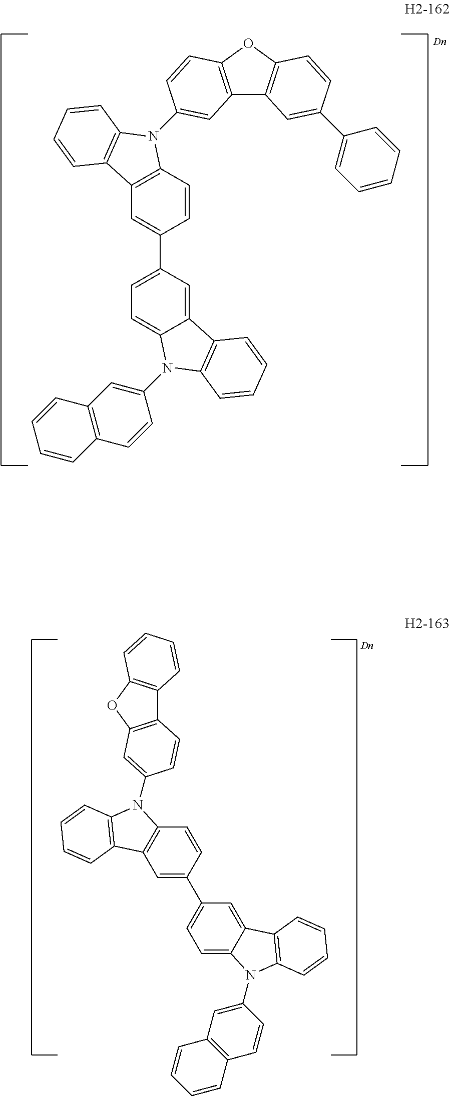 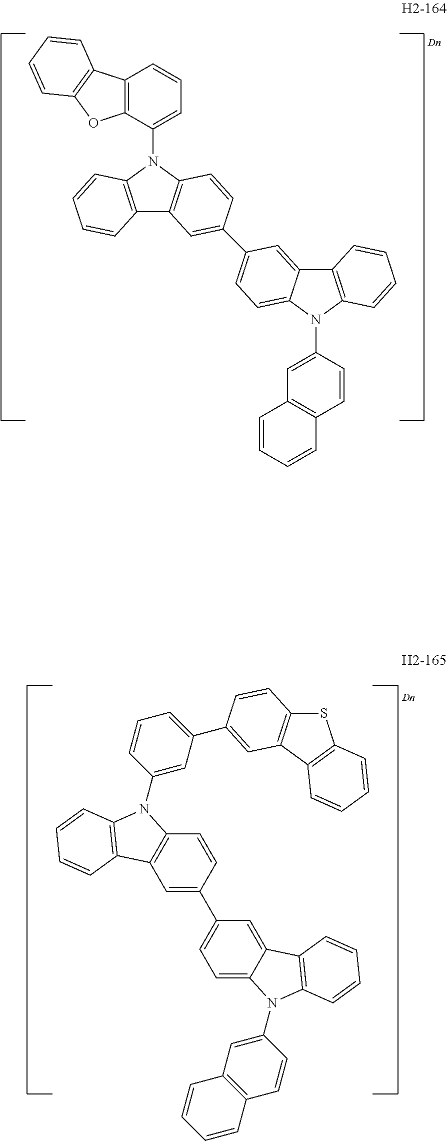 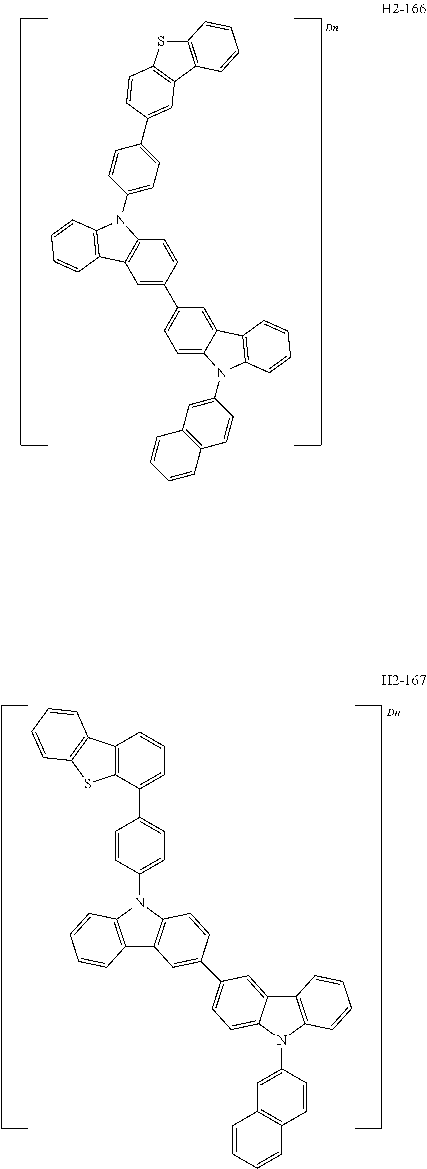 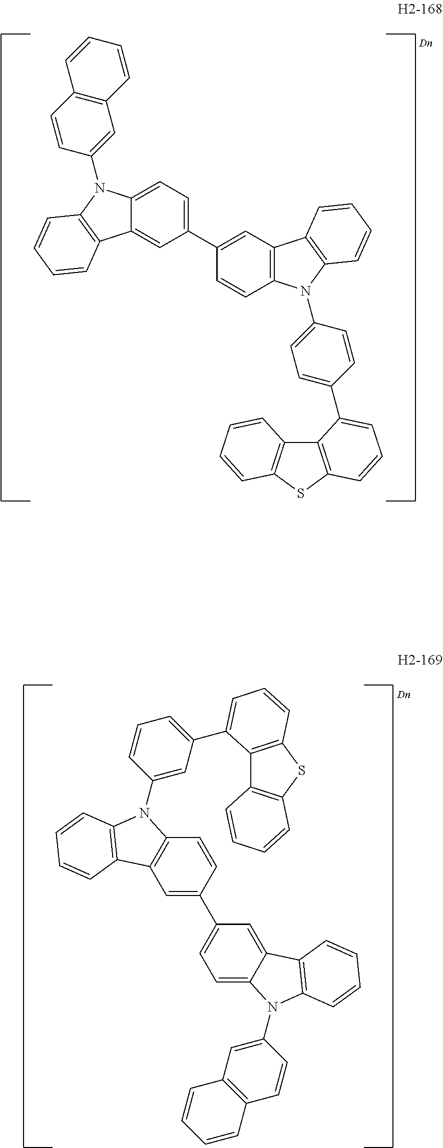 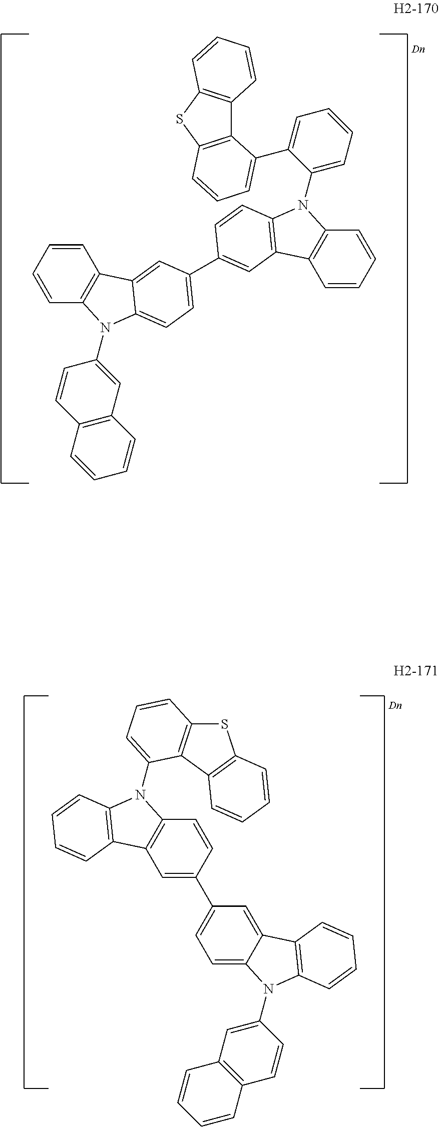 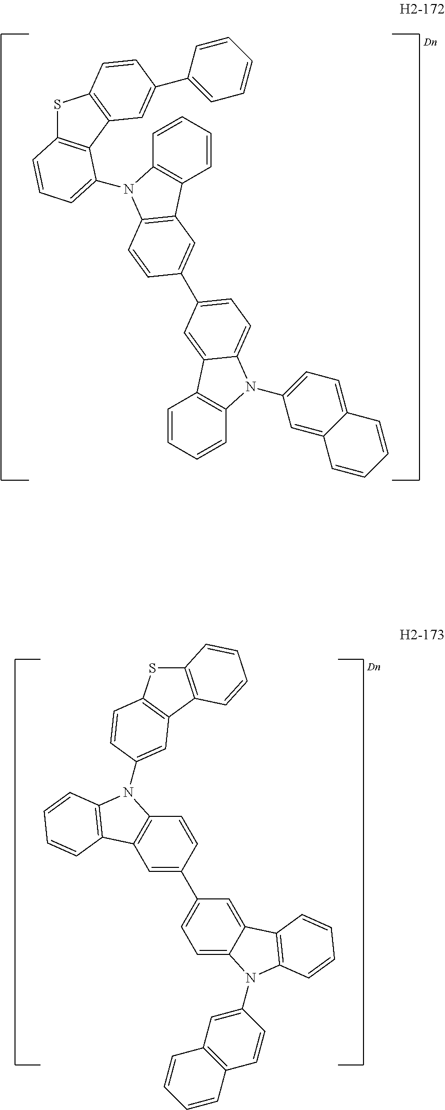 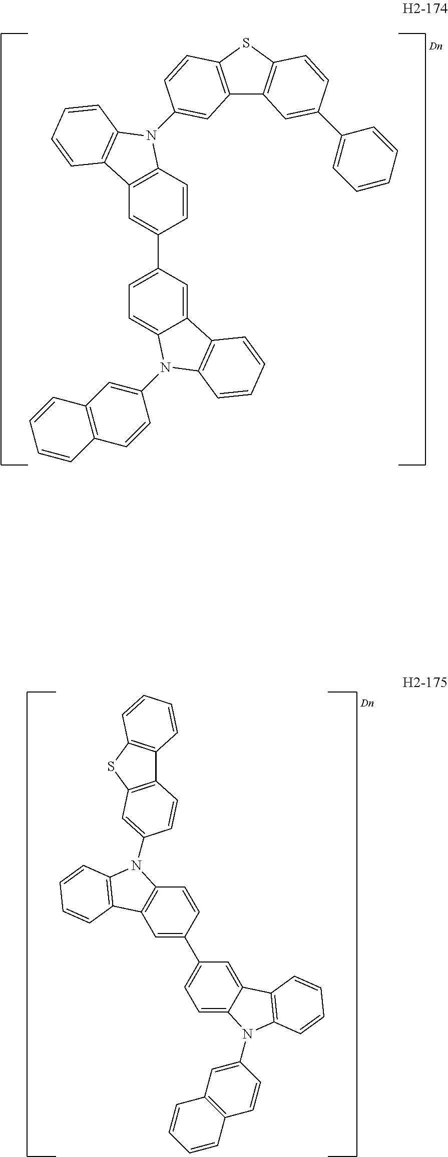 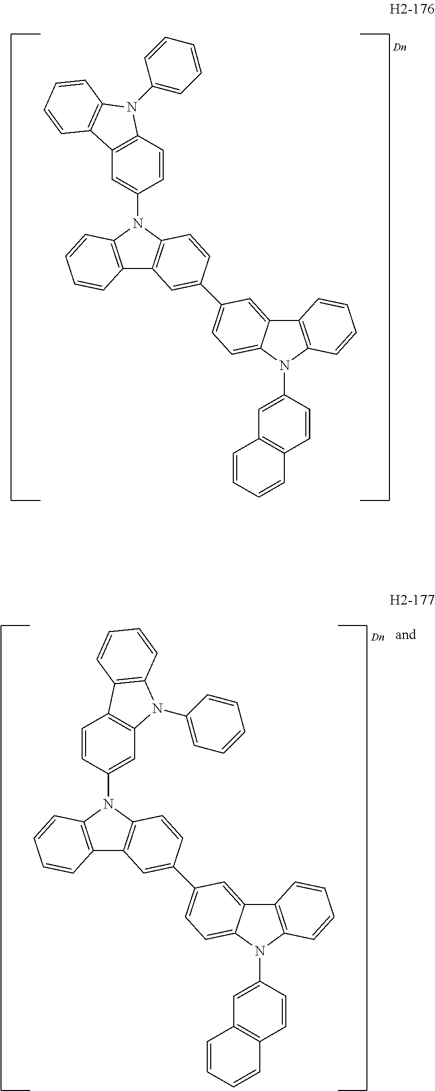 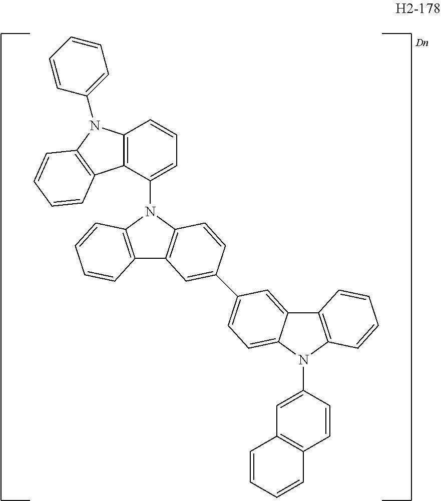
in the compounds above. Dₙ represents that n number of hydrogens are replaced with deuterium, and n is an integer of 1 or more and is not greater than the number of hydrogen in each compound.

10. An organic electroluminescent device comprising an anode, a cathode, and at least one light-emitting layer between the anode and the cathode, wherein at least one of the light-emitting layers comprises the plurality of host materials according to claim 1.

11. An organic electroluminescent compound represented by the following formula 4: 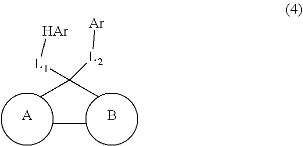
in formula 4,
ring A and ring B, each independently, represent a substituted or unsubstituted benzene ring, a substituted or unsubstituted naphthalene ring, or a substituted or unsubstituted phenanthrene ring;
L₁ represents a single bond;
L₂ represents a single bond, or a substituted or unsubstituted (C6-C30)arylene;
Ar represents a substituted or unsubstituted (C6-C30)aryl, a substituted or unsubstituted dibenzofuranyl, a substituted or unsubstituted dibenzothiophenyl, or a substituted or unsubstituted carbazolyl;
HAr represents a substituted or unsubstituted (3- to 20-membered)heteroaryl containing a nitrogen atom(s);
with the proviso that HAr is not a heteroaryl substituted with a substituent 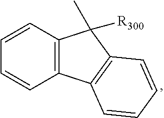
in which R₃₀₀ represents a (C1-C30)alkyl, or a (C6-C30)aryl, and * represents a bonding site of the substituent; and
the compound represented by formula 4 is not the following compounds:

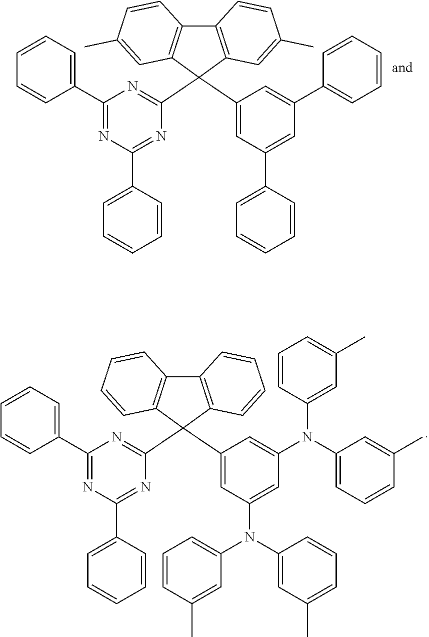 [SMILES: Cc1ccc2c(c1)C(c1cc(-c3ccccc3)cc(-c3ccccc3)c1)(c1nc(-c3ccccc3)nc(-c3ccccc3)n1)c1cc(C)ccc1-2 ; Cc1cccc(N(c2cccc(C)c2)c2cc(N(c3cccc(C)c3)c3cccc(C)c3)cc(C3(c4nc(-c5ccccc5)nc(-c5ccccc5)n4)c4ccccc4-c4ccccc43)c2)c1]

12. The organic electroluminescent compound according to claim 11, wherein the compound represented by formula 4 is selected from the following compounds: 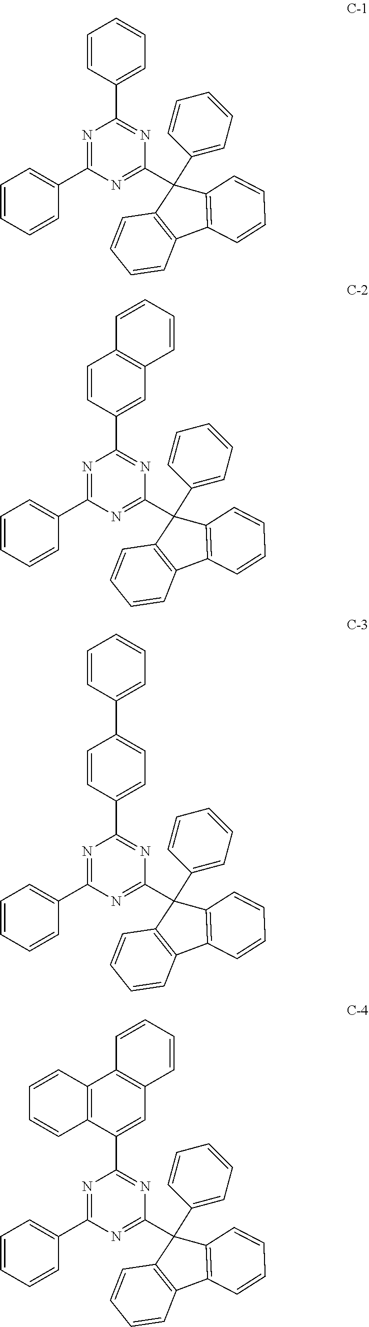 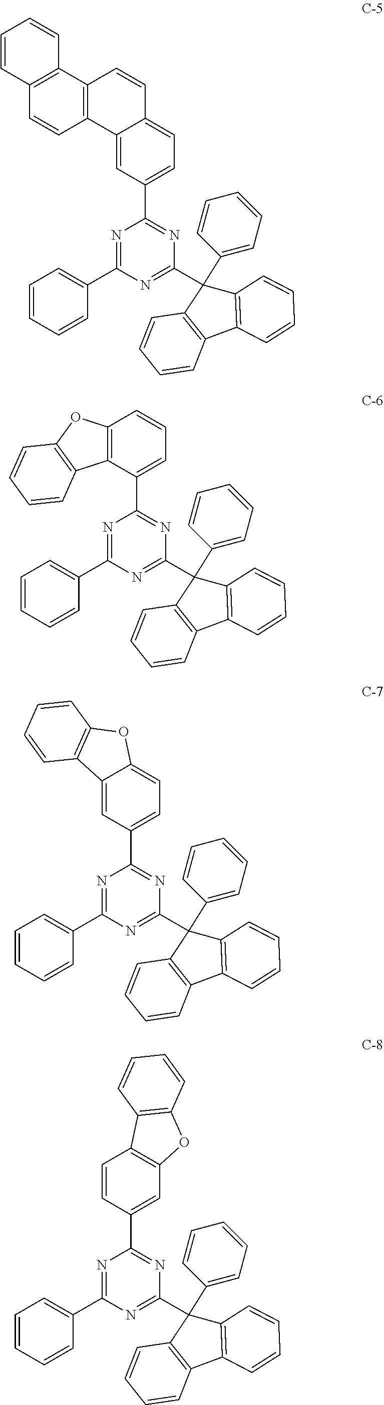 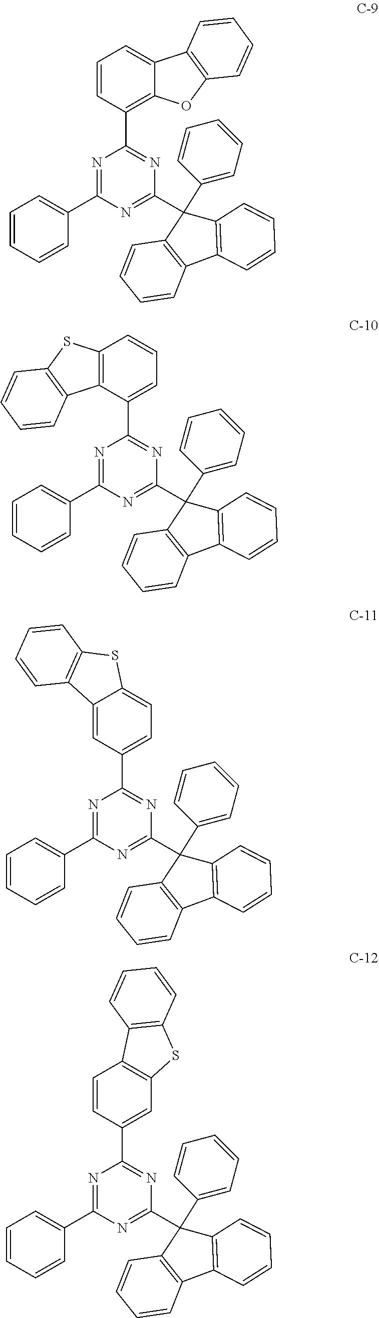 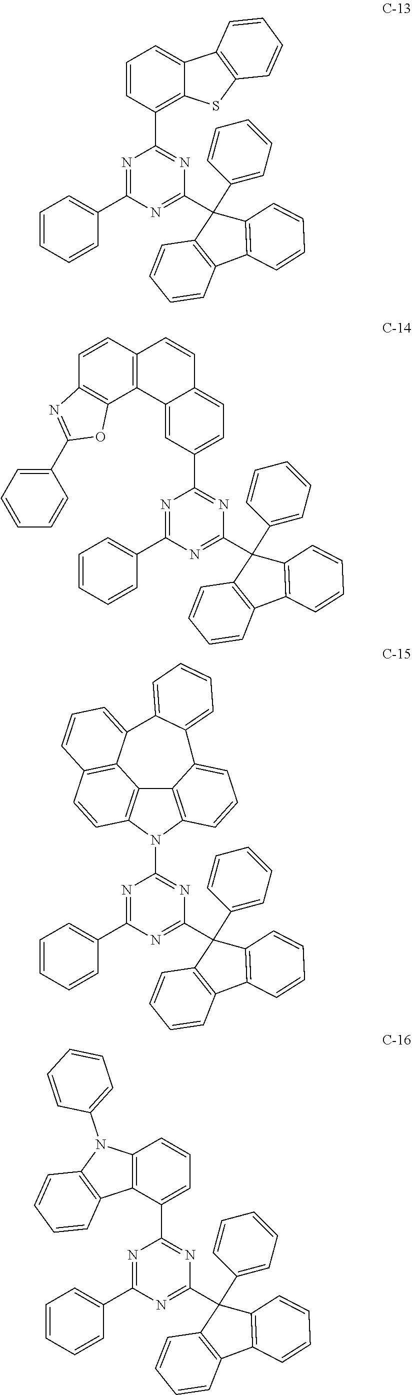 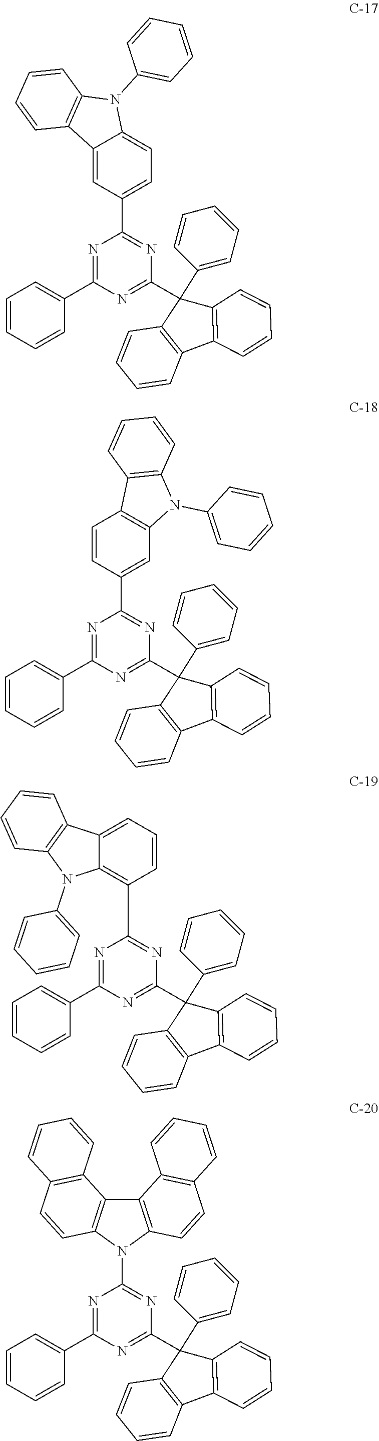 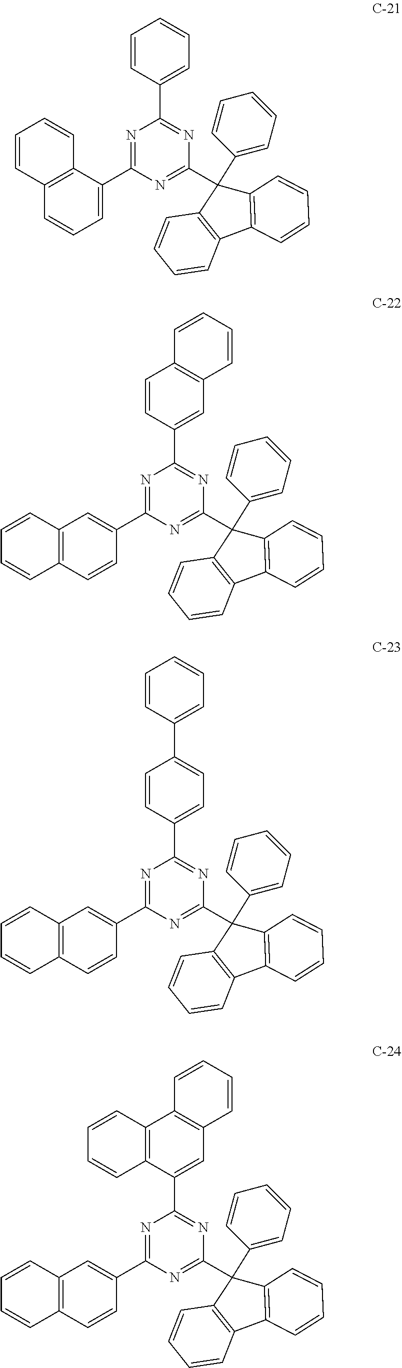 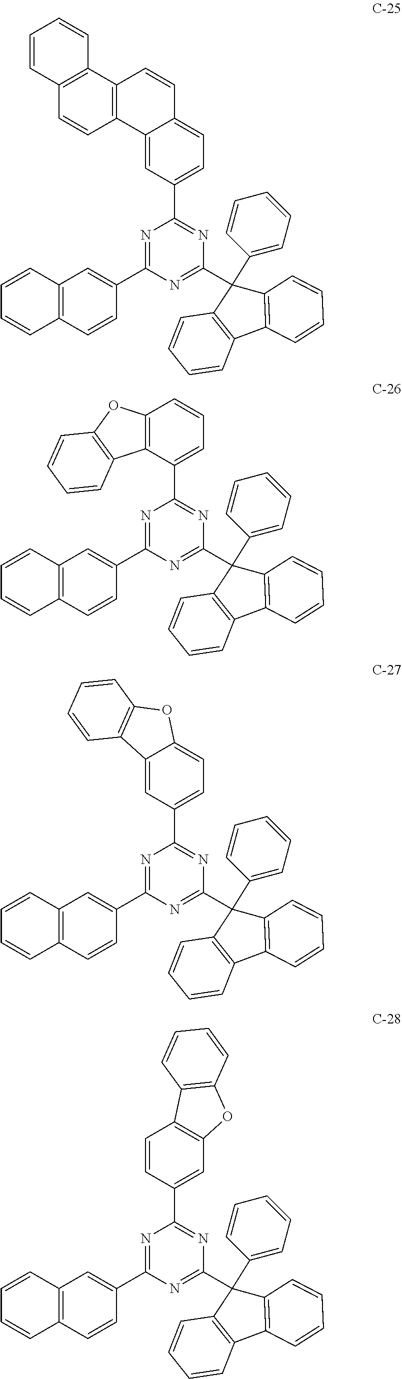 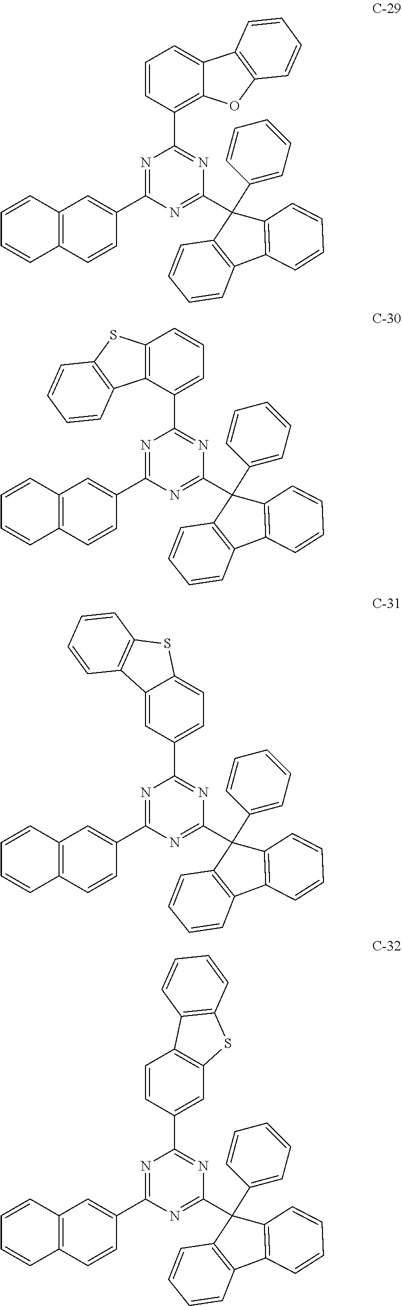 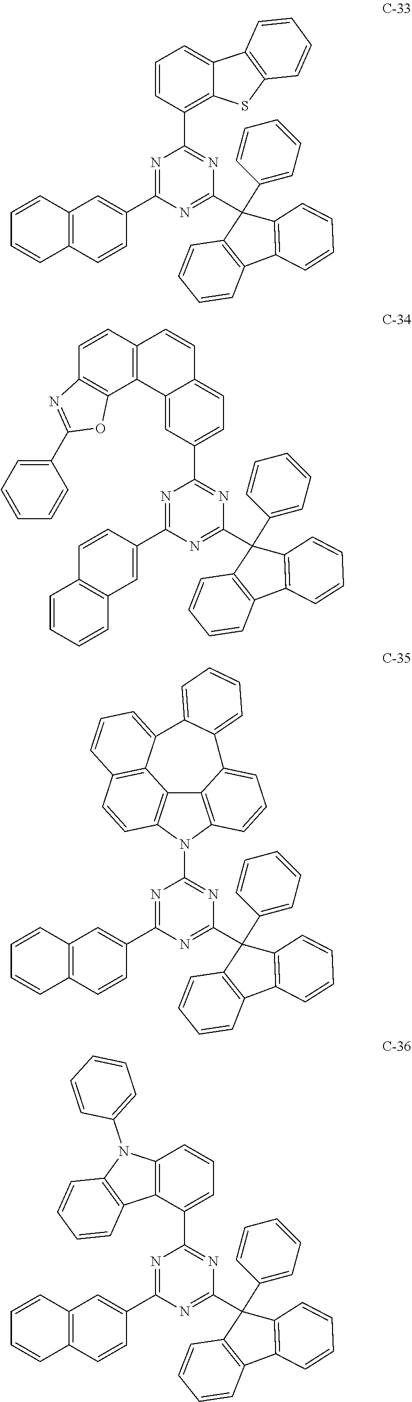 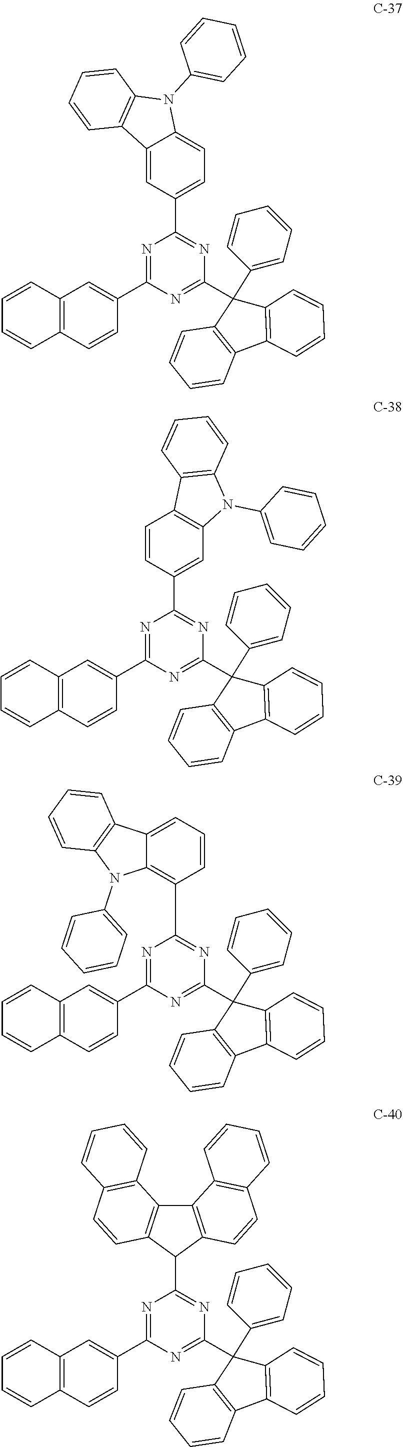 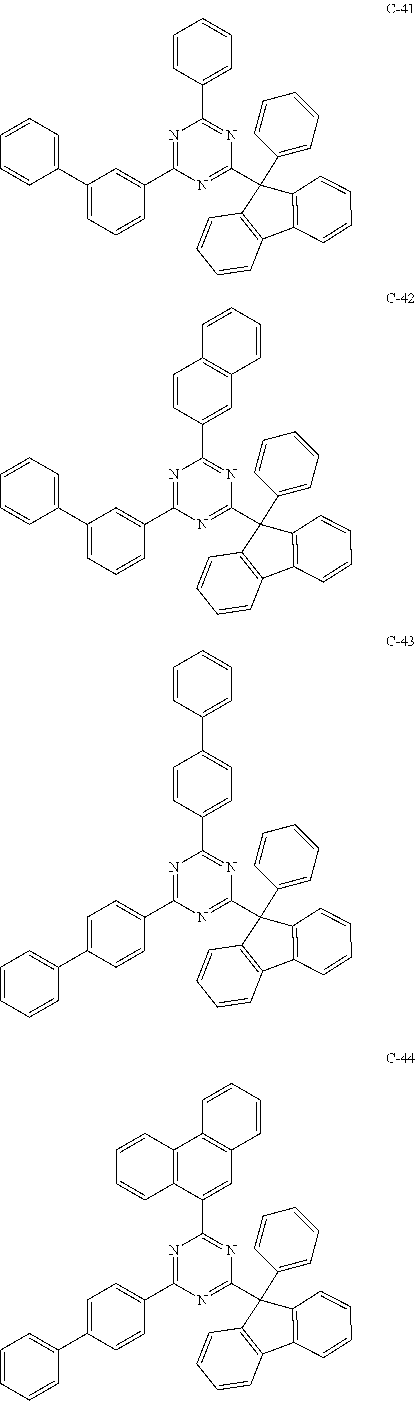 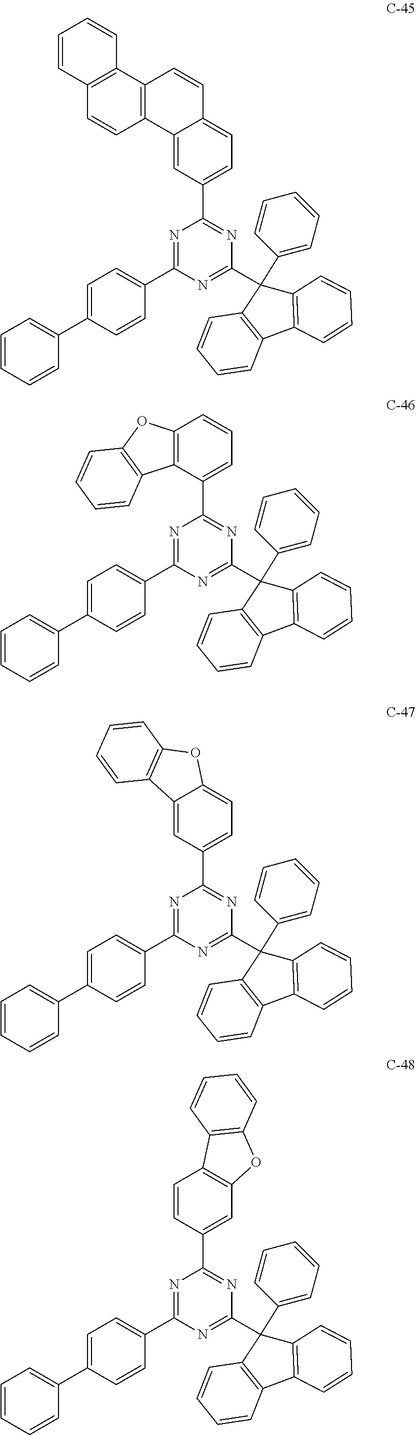 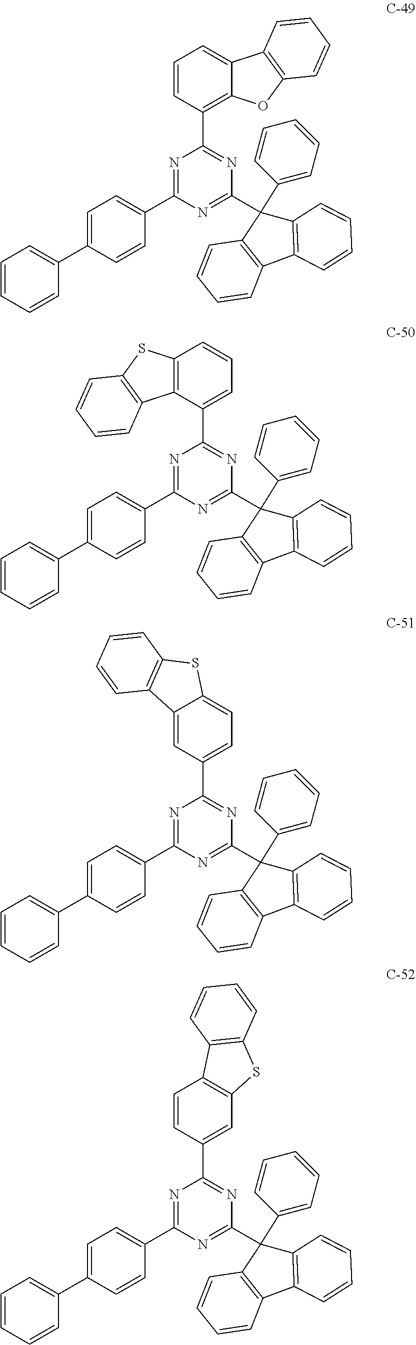 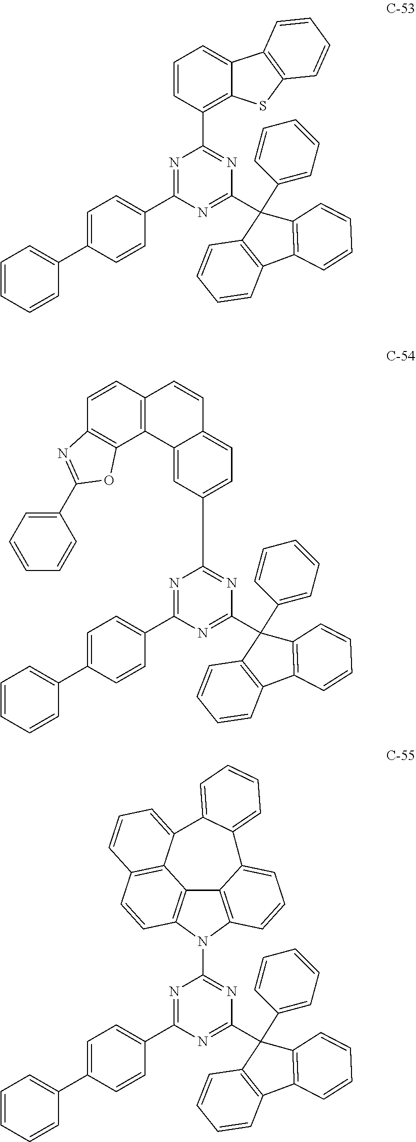 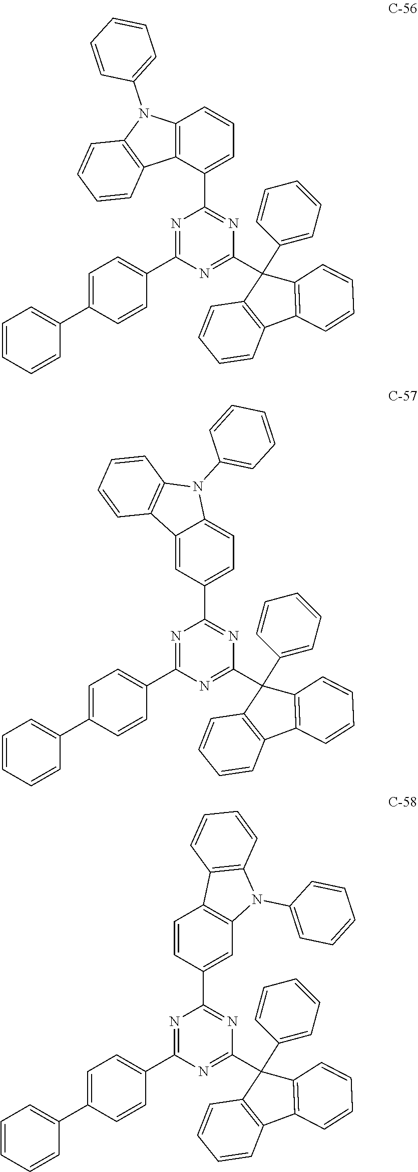 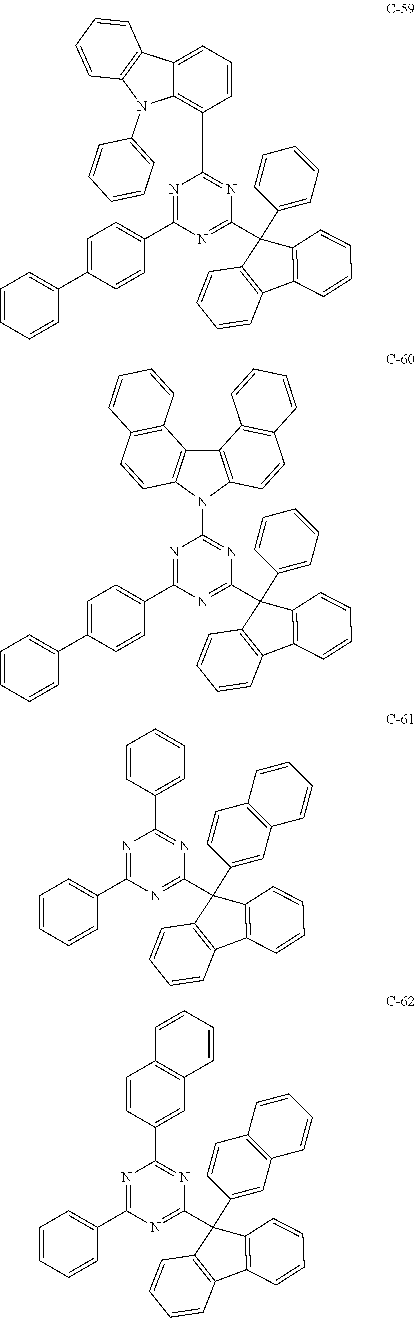 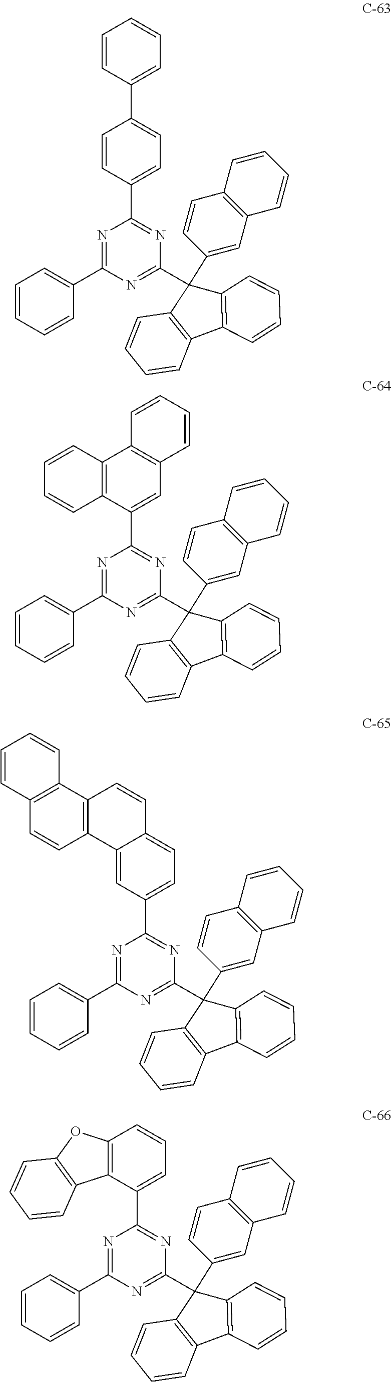 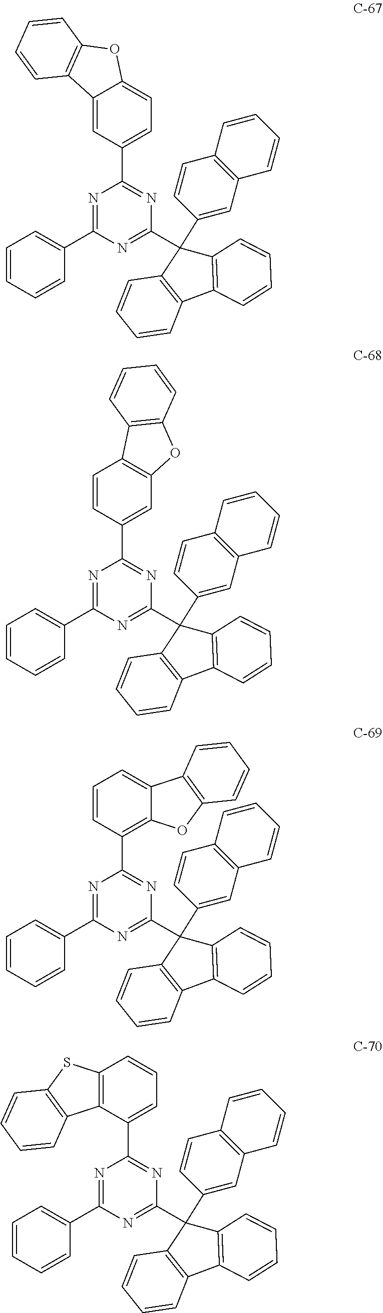 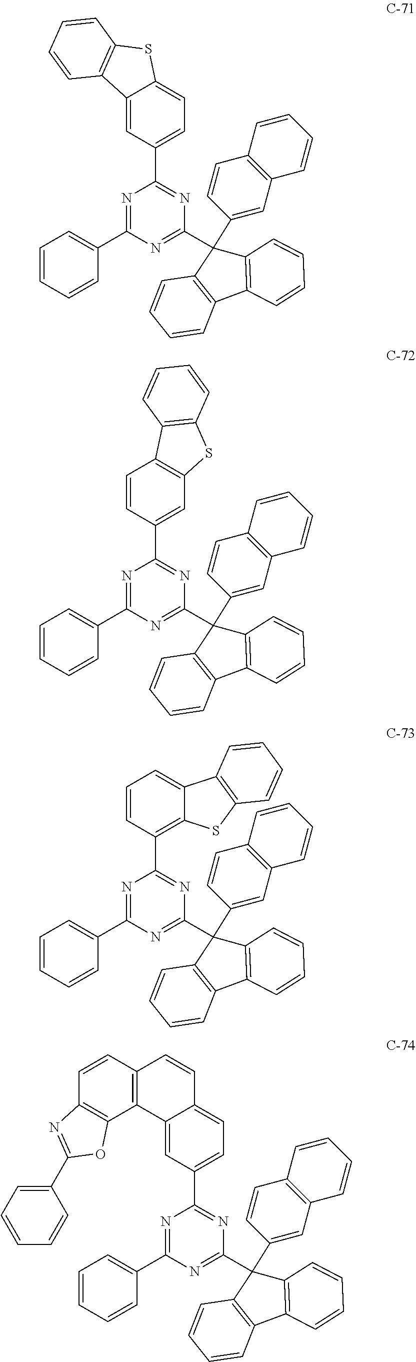 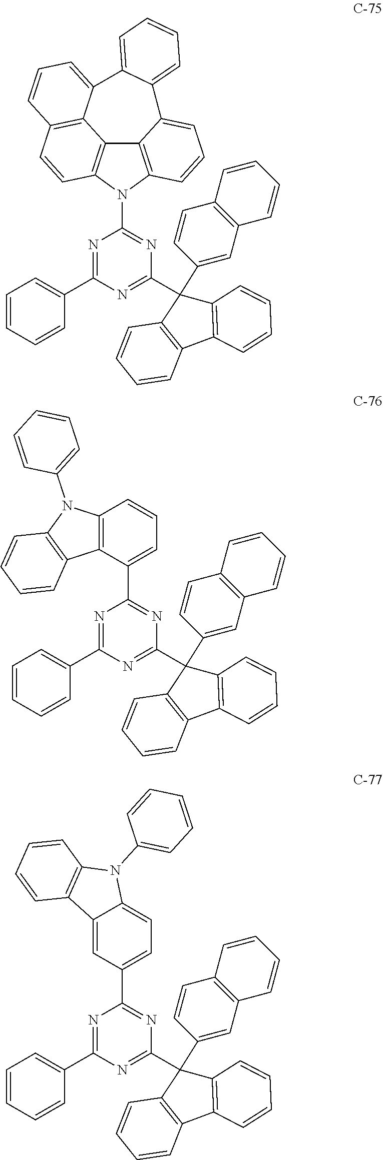 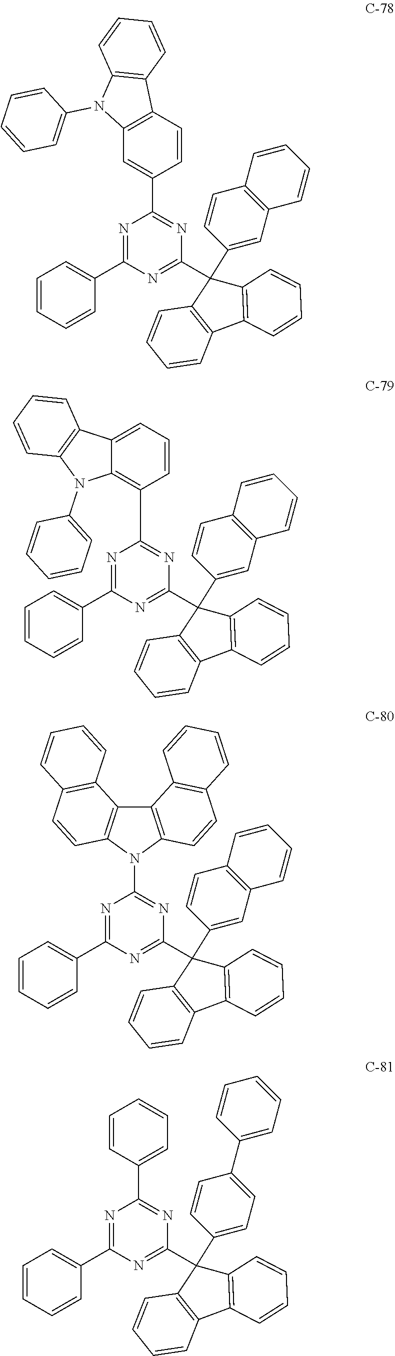 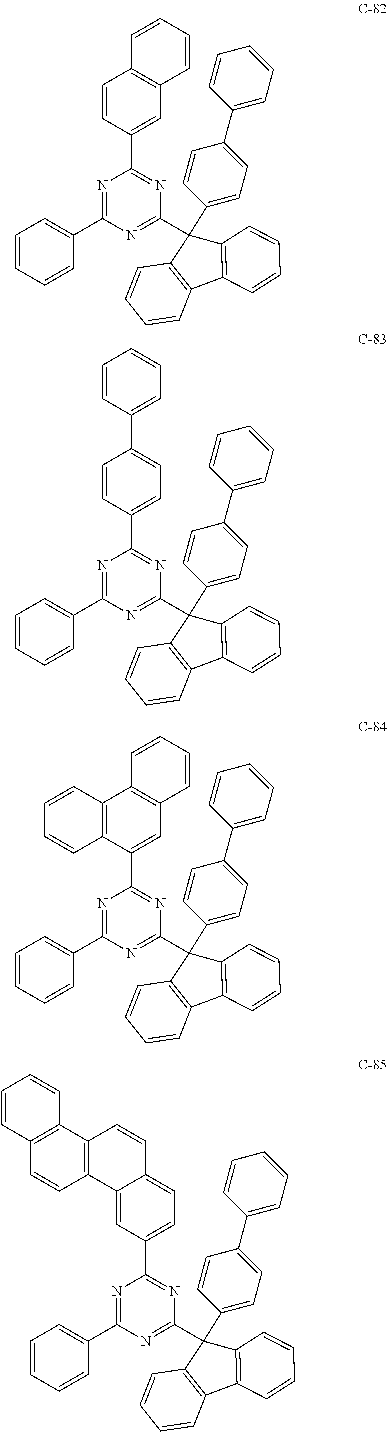 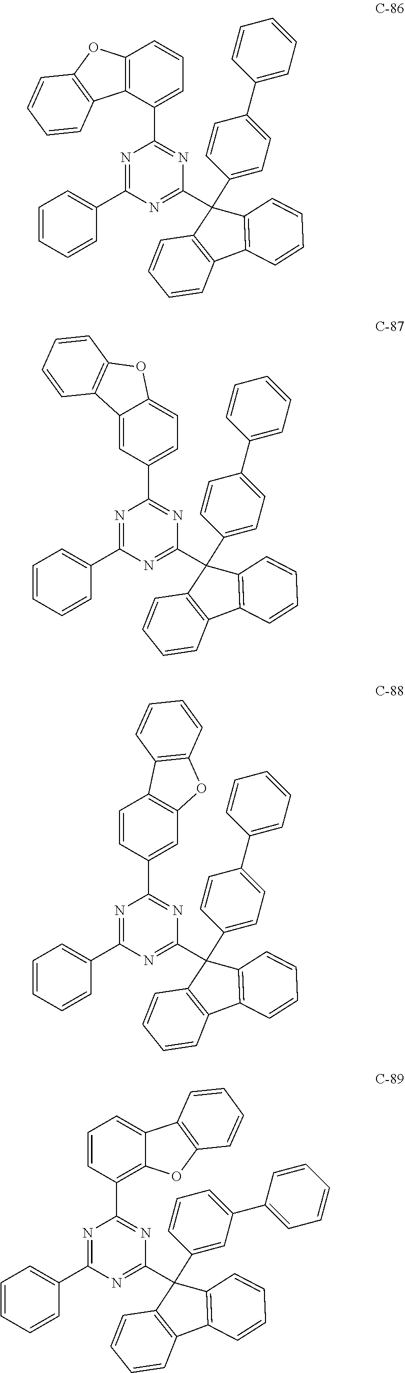 [SMILES: c1ccc(-c2ccc(C3(c4nc(-c5ccccc5)nc(-c5cccc6oc7ccccc7c56)n4)c4ccccc4-c4ccccc43)cc2)cc1 ; c1ccc(-c2ccc(C3(c4nc(-c5ccccc5)nc(-c5ccc6oc7ccccc7c6c5)n4)c4ccccc4-c4ccccc43)cc2)cc1 ; c1ccc(-c2ccc(C3(c4nc(-c5ccccc5)nc(-c5ccc6c(c5)oc5ccccc56)n4)c4ccccc4-c4ccccc43)cc2)cc1 ; c1ccc(-c2cccc(C3(c4nc(-c5ccccc5)nc(-c5cccc6c5oc5ccccc56)n4)c4ccccc4-c4ccccc43)c2)cc1] 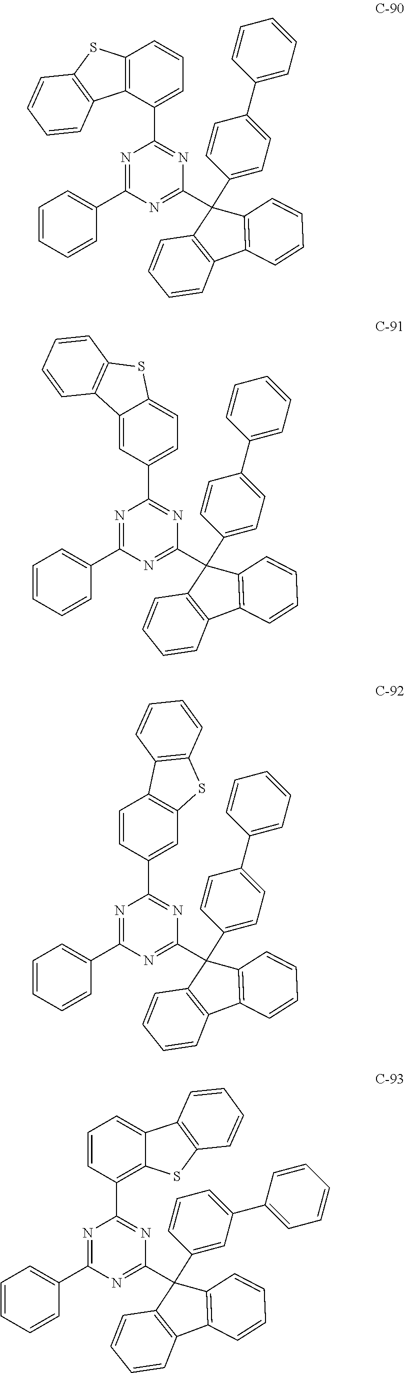 [SMILES: c1ccc(-c2ccc(C3(c4nc(-c5ccccc5)nc(-c5cccc6sc7ccccc7c56)n4)c4ccccc4-c4ccccc43)cc2)cc1 ; c1ccc(-c2ccc(C3(c4nc(-c5ccccc5)nc(-c5ccc6sc7ccccc7c6c5)n4)c4ccccc4-c4ccccc43)cc2)cc1 ; c1ccc(-c2ccc(C3(c4nc(-c5ccccc5)nc(-c5ccc6c(c5)sc5ccccc56)n4)c4ccccc4-c4ccccc43)cc2)cc1 ; c1ccc(-c2cccc(C3(c4nc(-c5ccccc5)nc(-c5cccc6c5sc5ccccc56)n4)c4ccccc4-c4ccccc43)c2)cc1] 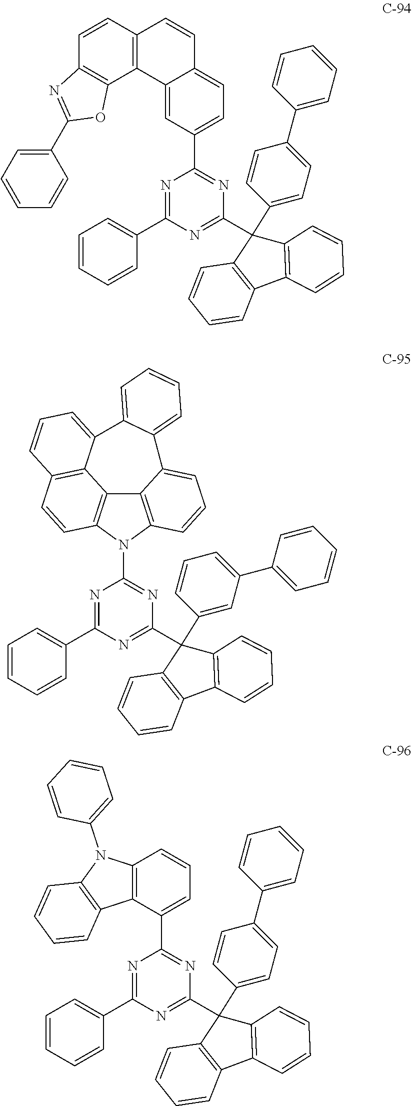 [SMILES: c1ccc(-c2ccc(C3(c4nc(-c5ccccc5)nc(-c5ccc6ccc7ccc8nc(-c9ccccc9)oc8c7c6c5)n4)c4ccccc4-c4ccccc43)cc2)cc1 ; c1ccc(-c2cccc(C3(c4nc(-c5ccccc5)nc(-n5c6cccc7c6c6c8c(cccc8ccc65)-c5ccccc5-7)n4)c4ccccc4-c4ccccc43)c2)cc1 ; c1ccc(-c2ccc(C3(c4nc(-c5ccccc5)nc(-c5cccc6c5c5ccccc5n6-c5ccccc5)n4)c4ccccc4-c4ccccc43)cc2)cc1] 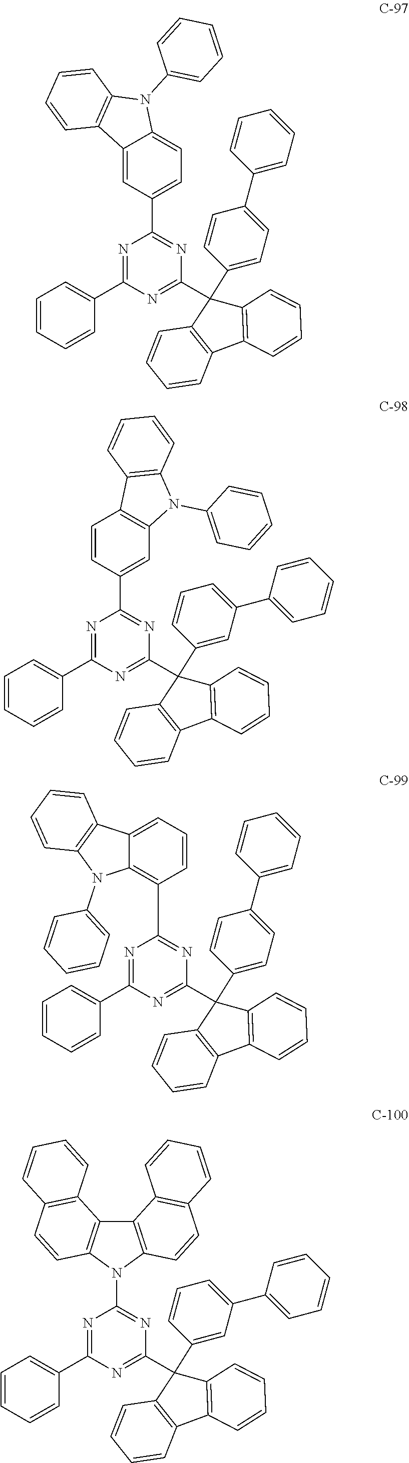 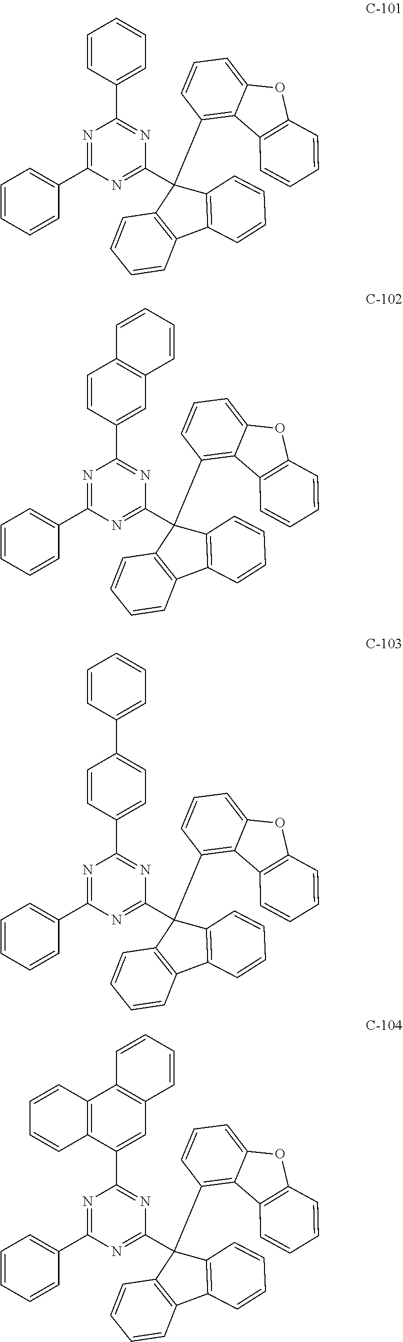 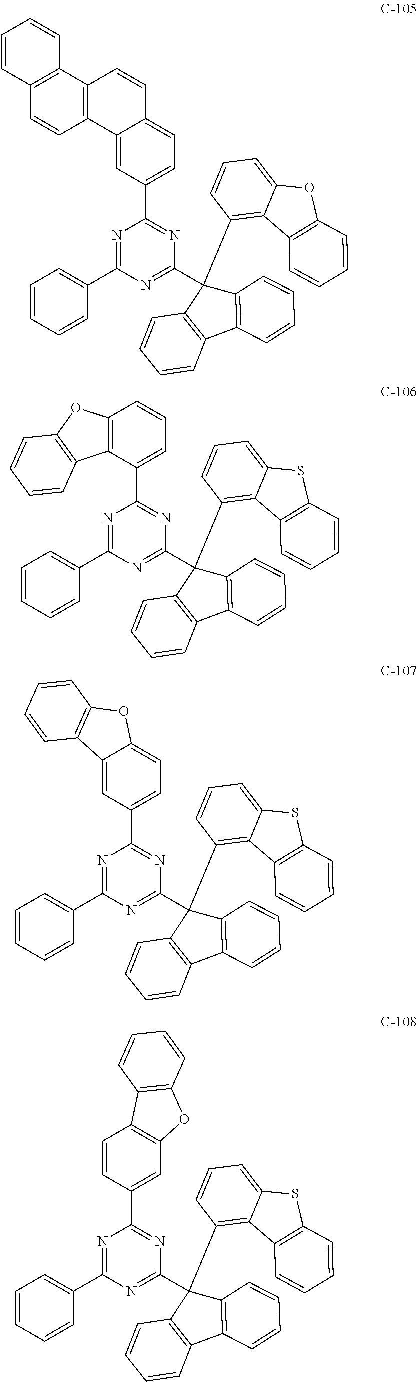 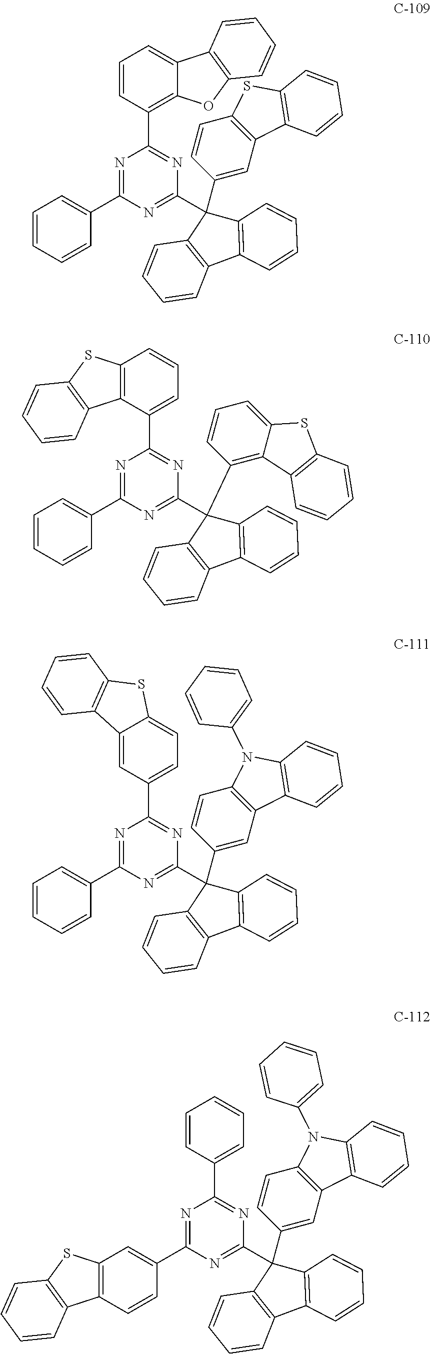 [SMILES: c1ccc(-c2nc(-c3cccc4c3oc3ccccc34)nc(C3(c4ccc5sc6ccccc6c5c4)c4ccccc4-c4ccccc43)n2)cc1 ; c1ccc(-c2nc(-c3cccc4sc5ccccc5c34)nc(C3(c4cccc5sc6ccccc6c45)c4ccccc4-c4ccccc43)n2)cc1 ; c1ccc(-c2nc(-c3ccc4sc5ccccc5c4c3)nc(C3(c4ccc5c(c4)c4ccccc4n5-c4ccccc4)c4ccccc4-c4ccccc43)n2)cc1 ; c1ccc(-c2nc(-c3ccc4c(c3)sc3ccccc34)nc(C3(c4ccc5c(c4)c4ccccc4n5-c4ccccc4)c4ccccc4-c4ccccc43)n2)cc1] 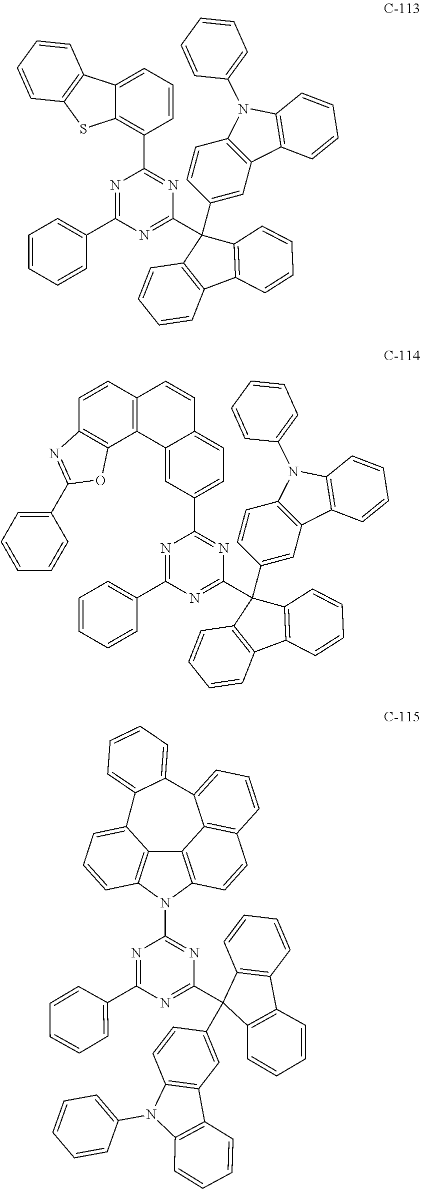 [SMILES: c1ccc(-c2nc(-c3cccc4c3sc3ccccc34)nc(C3(c4ccc5c(c4)c4ccccc4n5-c4ccccc4)c4ccccc4-c4ccccc43)n2)cc1 ; c1ccc(-c2nc(-c3ccc4ccc5ccc6nc(-c7ccccc7)oc6c5c4c3)nc(C3(c4ccc5c(c4)c4ccccc4n5-c4ccccc4)c4ccccc4-c4ccccc43)n2)cc1 ; c1ccc(-c2nc(-n3c4cccc5c4c4c6c(cccc6ccc43)-c3ccccc3-5)nc(C3(c4ccc5c(c4)c4ccccc4n5-c4ccccc4)c4ccccc4-c4ccccc43)n2)cc1] 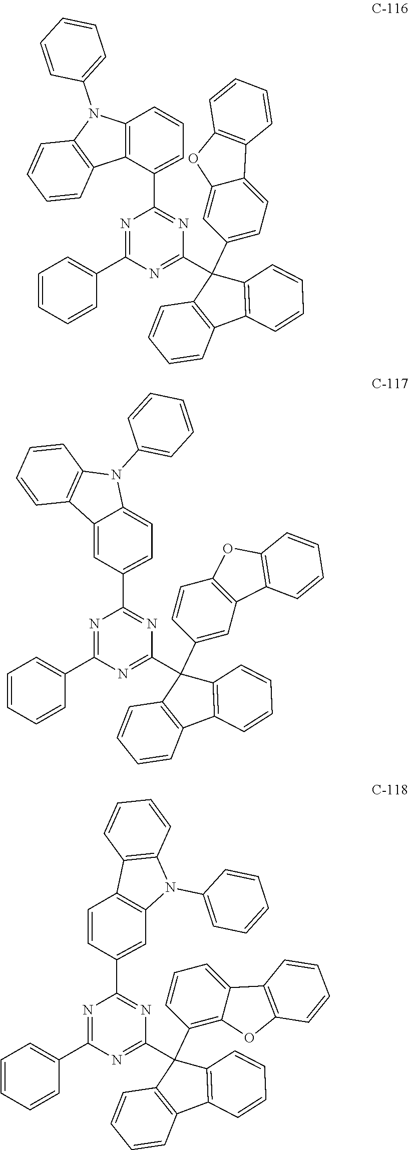 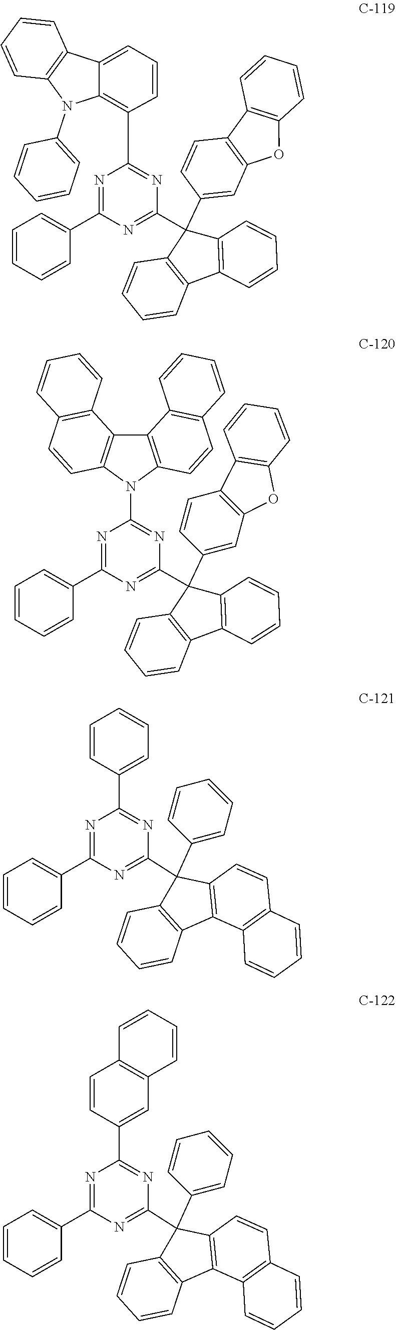 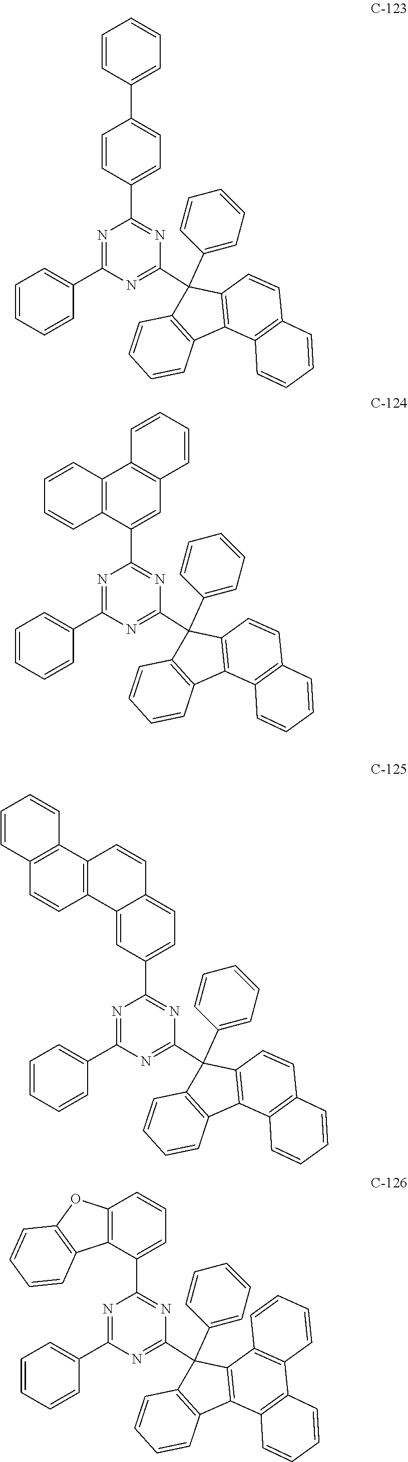 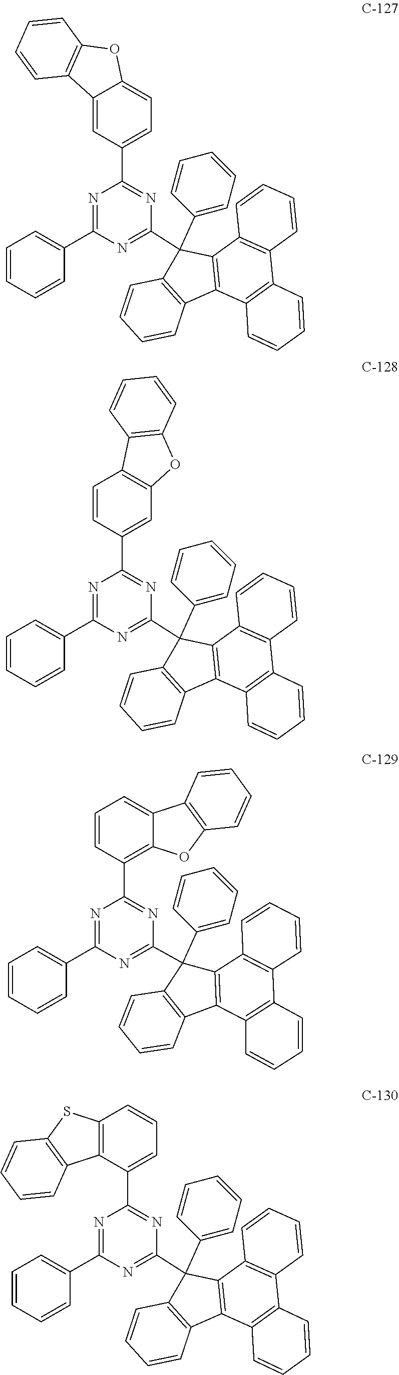 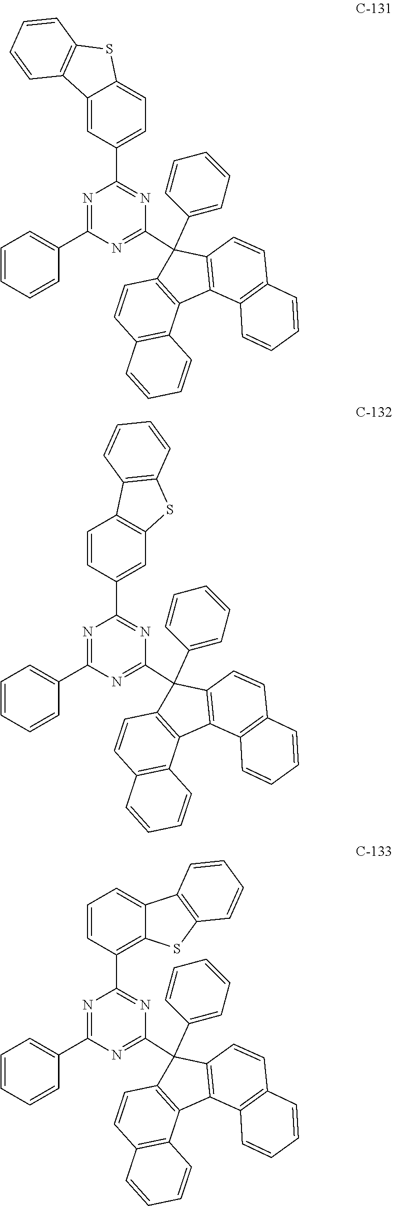 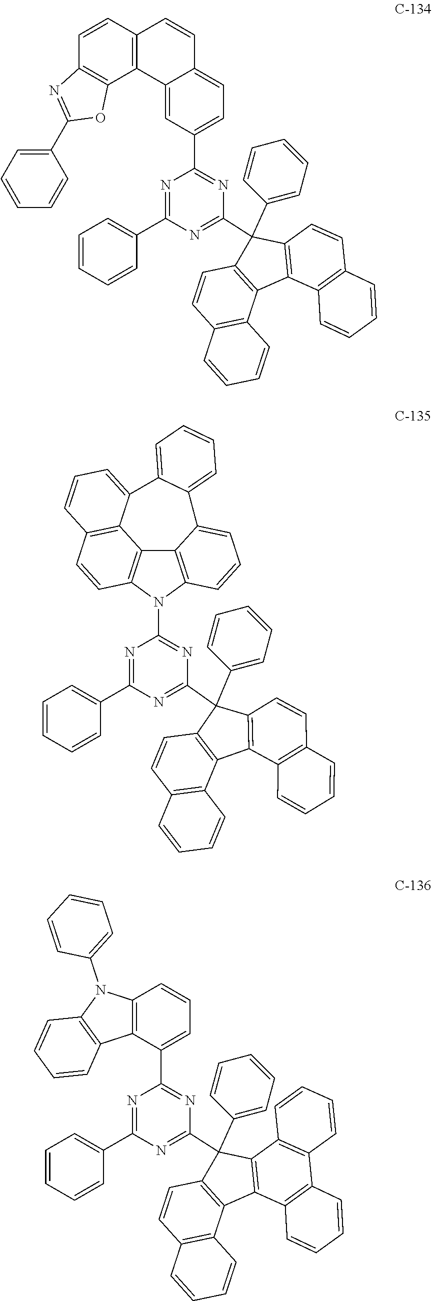 [SMILES: c1ccc(-c2nc(-c3ccc4ccc5ccc6nc(-c7ccccc7)oc6c5c4c3)nc(C3(c4ccccc4)c4ccc5ccccc5c4-c4c3ccc3ccccc43)n2)cc1 ; c1ccc(-c2nc(-n3c4cccc5c4c4c6c(cccc6ccc43)-c3ccccc3-5)nc(C3(c4ccccc4)c4ccc5ccccc5c4-c4c3ccc3ccccc43)n2)cc1 ; c1ccc(-c2nc(-c3cccc4c3c3ccccc3n4-c3ccccc3)nc(C3(c4ccccc4)c4ccc5ccccc5c4-c4c3c3ccccc3c3ccccc43)n2)cc1] 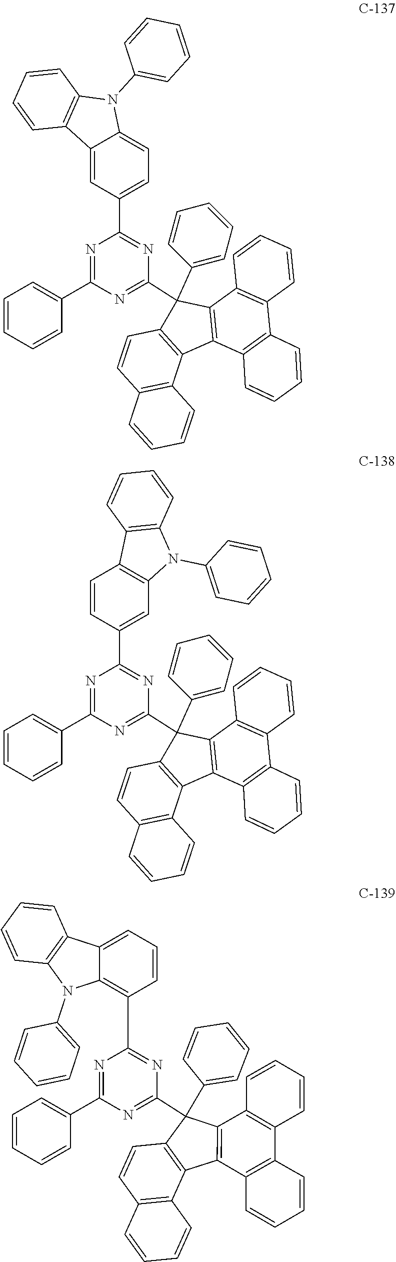 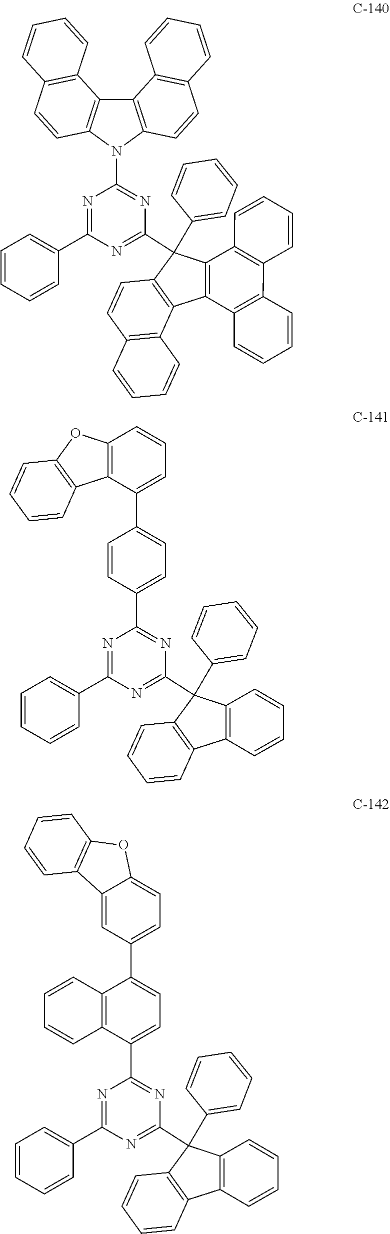 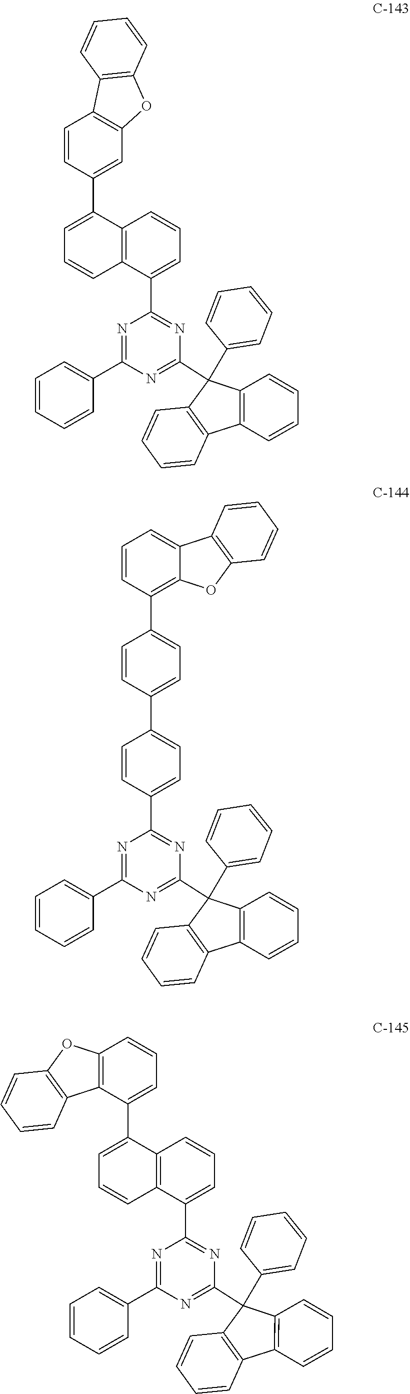 [SMILES: c1ccc(-c2nc(-c3cccc4c(-c5ccc6c(c5)oc5ccccc56)cccc34)nc(C3(c4ccccc4)c4ccccc4-c4ccccc43)n2)cc1 ; c1ccc(-c2nc(-c3ccc(-c4ccc(-c5cccc6c5oc5ccccc56)cc4)cc3)nc(C3(c4ccccc4)c4ccccc4-c4ccccc43)n2)cc1 ; c1ccc(-c2nc(-c3cccc4c(-c5cccc6oc7ccccc7c56)cccc34)nc(C3(c4ccccc4)c4ccccc4-c4ccccc43)n2)cc1] 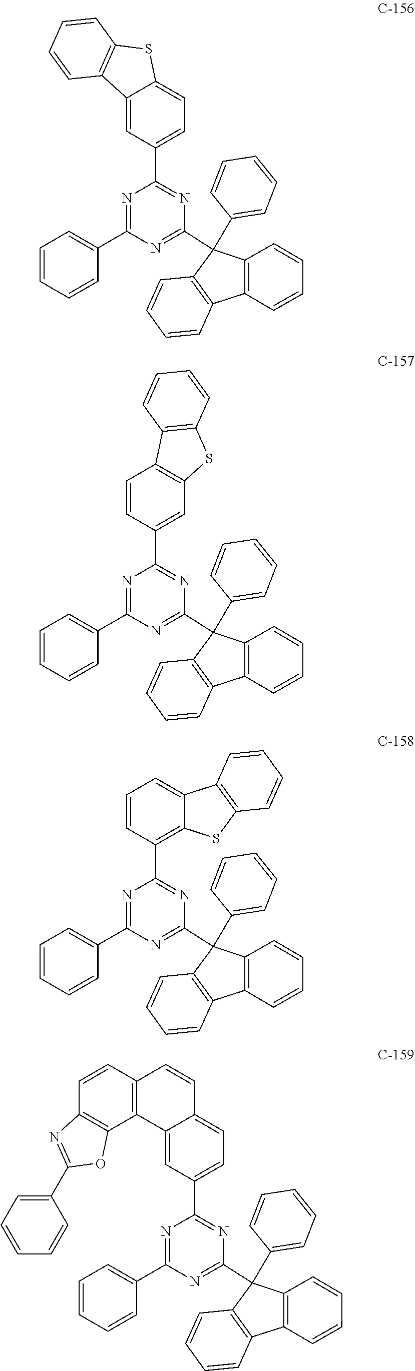 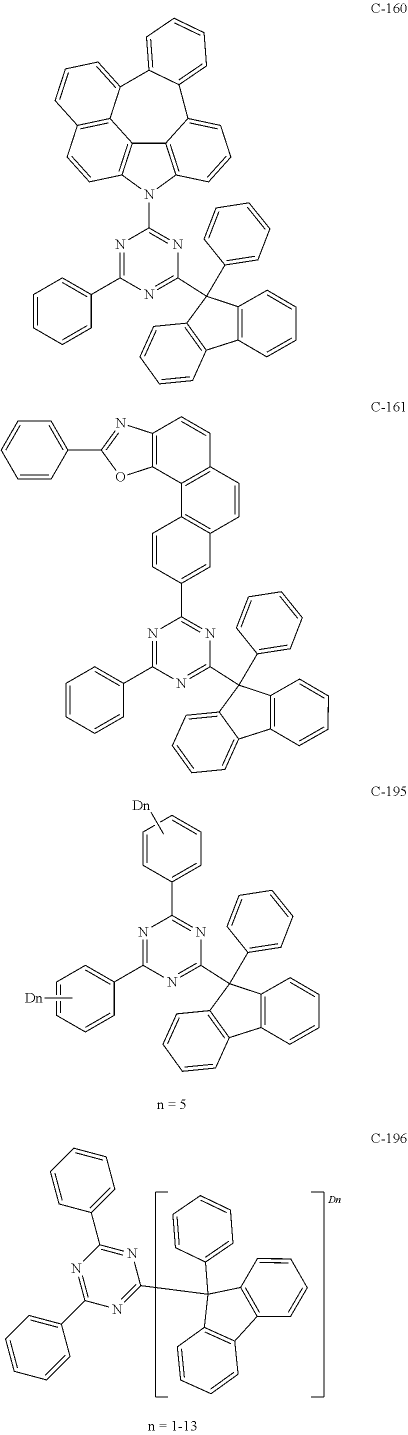 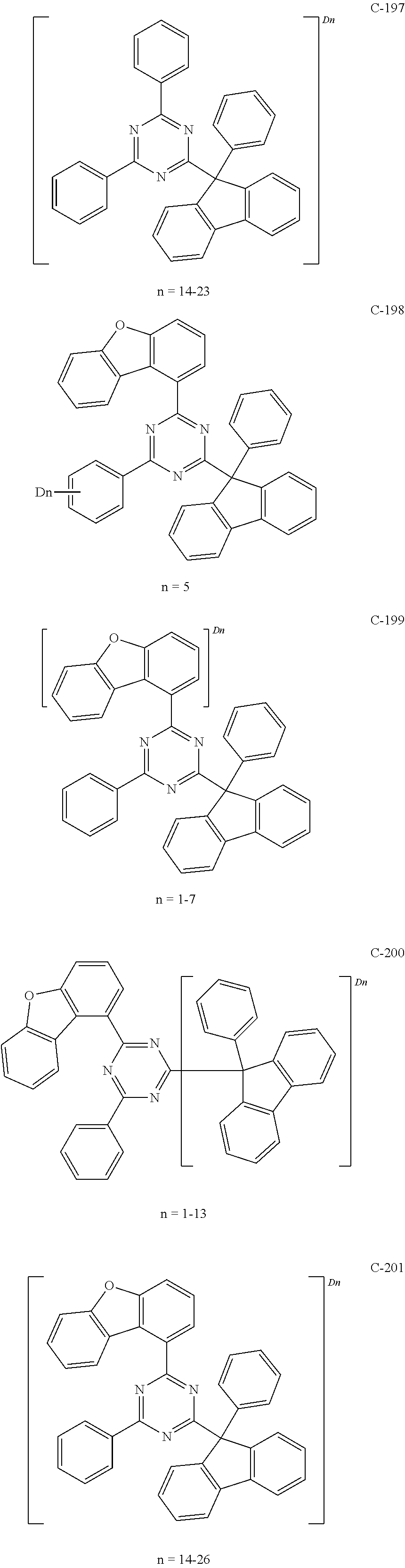 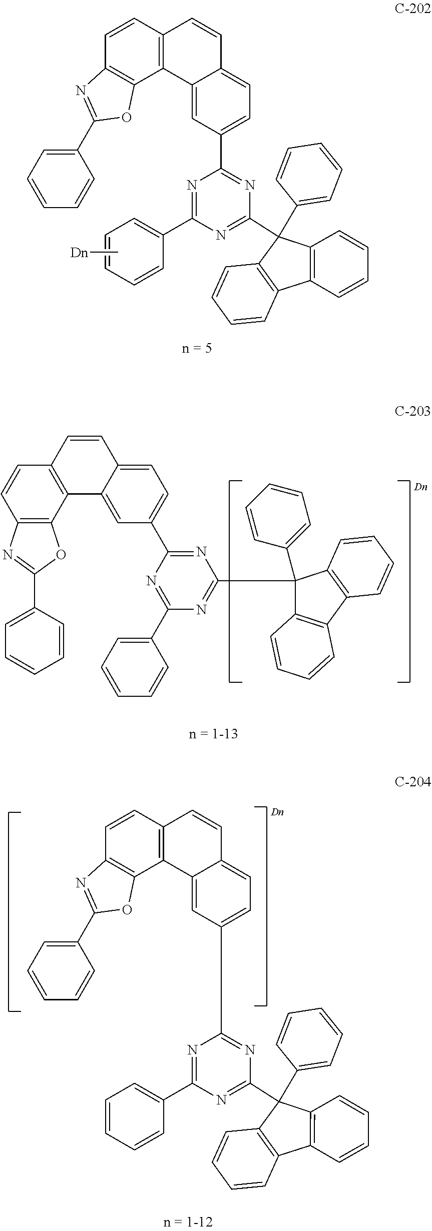 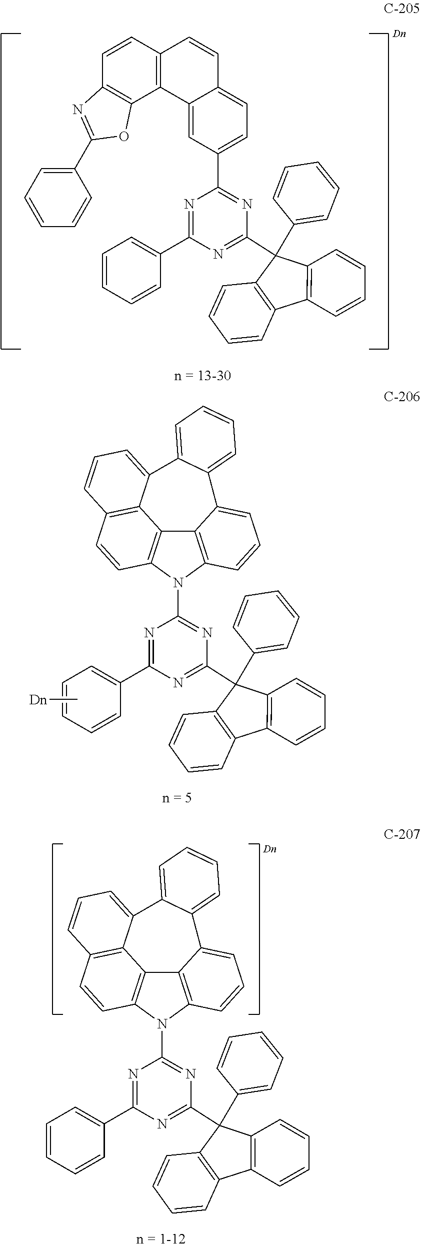 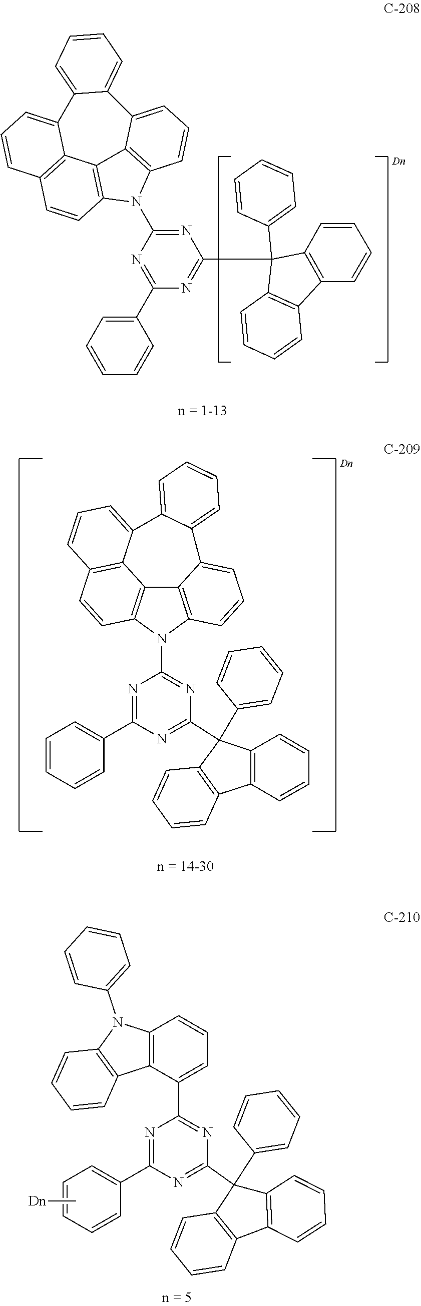 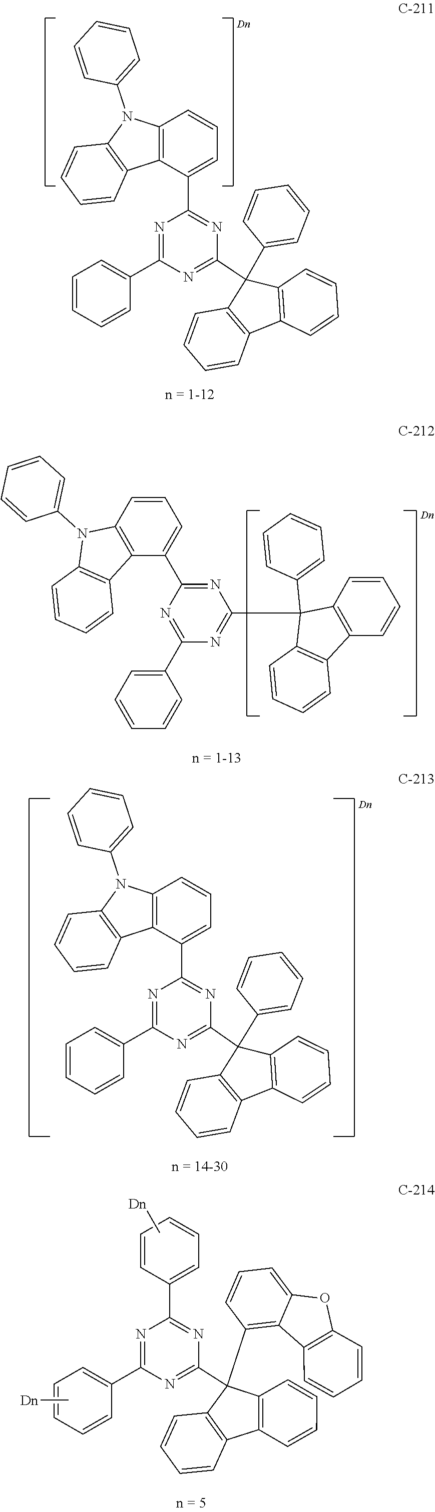 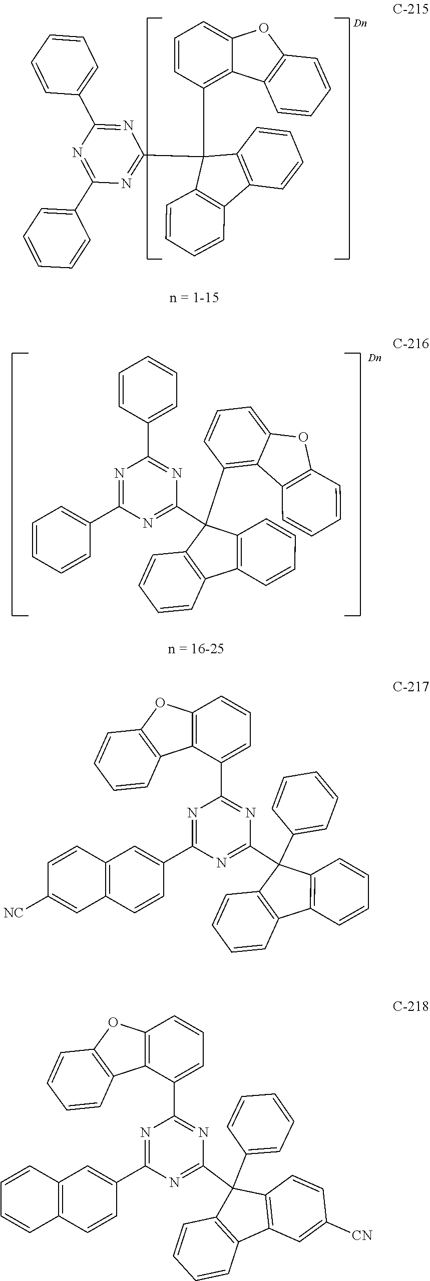 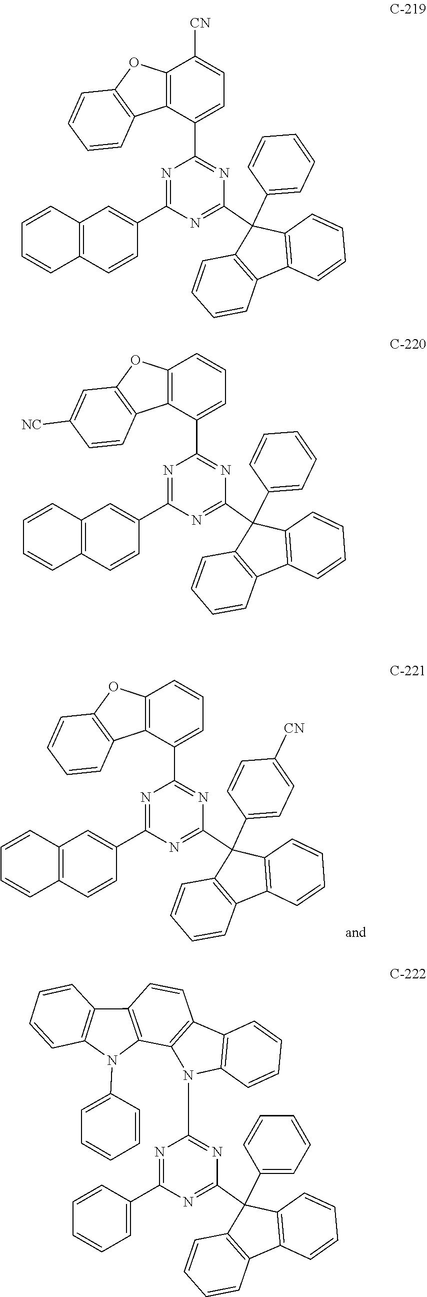
in the compounds above, D represents deuterium, and n represents the number of deuterium.

13. An organic electroluminescent material comprising the organic electroluminescent compound according to claim 11.

14. An organic electroluminescent device comprising the organic electroluminescent material according to claim 13.
